SEQUENCE LISTING (165 sequences)

SEQ ID NO: 1 - DNA, 11177 bp, synthetic construct
  source 1..11177: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggaccgacagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctcagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctcctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtcctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccacacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgacggagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtaccggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacgcccgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaaccgggtgatcaccagggaggagctggaggccctgacccctagcaggacccctagcaggagcgtgagcaggaccagcctggtgagcaaccctcccggcgtgaaccgggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtcctggagaggacggagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctccaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtcctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaaccgggtgggcatcctgagcgagctgtgcaaggccgtggagagcaggtacgagacagtgggcaccagcatcatcgtgatggccatgaccaccctggccagcagcgtcaagtccttcagctacctgaggggggcccctataactctctacggctaacctgaatggactacgacatagtctagtccgccaaggccgccaccatgctgcctagcaccatccagaccctgacactgttcctgaccagcggcggcgtgctgctgagcctgtacgtgagcgccagcctgagctacctgctgtactccgacatcctgctgaagttcagccagaccgagatcaccgcccctaccatgcccctggactgcgccaacgccagcaacgtgcaggccgtgaacaggagcgccaccaagggcgccaccctgctgctccccgagcccgagtggacctaccccaggctgagctgccccggcagcaccttccagaaggccctgctgatcagccctcacaggttcggcgagacaaagggcaacagcgcccctctgatcatcagggagcccttcgtggcctgcggccccaacgagtgcaagcacttcgccctgacccactacgccgcccagcccggcggctactacaacggcaccaggggcgacaggaacaagctgaggcacctgatcagcgtgaagctgggcaagatccccaccgtggagaacagcatcttccacatggccgcctggagcggcagcgcctgccacgacggcaaggagtggacctacatcggcgtggacggccccgacaacaacgccctgctgaaggtgaagtacggcgaggcctacaccgacacctaccacagctacgccaacaacatcctgaggacccaggagagcgcctgcaactgcatcggcggcaactgctacctgatgatcaccgacggcagcgccagcggcgtgagcgagtgcaggttcctgaagatcagggagggcaggatcatcaaggagatcttccccaccggcagggtgaagcacaccgaggagtgcacctgcggcttcgccagcaacaagaccatcgagtgcgcctgcagggacaacaggtacaccgccaagaggcccttcgtgaagctgaacgtggagacagacaccgccgagatcaggctgatgtgcaccgacacctacctggacacccctaggcccaacgacggcagcatcaccggcccctgcgagagcgacggcgacgagggcagcggcggcatcaagggcggcttcgtgcaccagaggatgaagtccaagatcggcaggtggtacagcaggaccatgagcaagaccgagaggatgggcatgggcctgtacgtgaagtacggcggcgacccctgggccgacagcgacgccctggtgttcagcggcgtgatgatcagcatgaaggagcccggctggtacagcttcggcttcgagatcaaggacaagaagtgcgacgtgccctgcatcggcatcgagatggtgcacgacggcggcaaggagacatggcacagcgccgccaccgccatctactgcctgatgggcagcggccagctgctgtgggacaccatcaccggcgtggacatggccctgtgatagtaaactcgaagcagtgttaaatcattcagctacctgagaggggcccctataactctctacggctaacctgaatggactacgacatagtctagtccgccaaggccgccaccatgaaggccatcatcgtgctgctgatggtggtgacctctaacgccgacaggatctgcaccggcatcaccagcagcaacagccctcacgtggtgaagaccgccacccagggcgaggtgaacgtgaccggcgtgatccctctgaccaccacacccaccaagagccacttcgccaacctgaagggcaccgagacaaggggcaagctgtgccccaagtgcctgaactgcaccgacctggacgtggccctgggcaggcccaagtgcaccggcaagatccccagcgccagggtgagcatcctgcacgaggtgaggcccgtgaccagcggctgcttccccatcatgcacgacaggaccaagatcaggcagctgcccaacctgctgaggggctacgagcacgtgaggctgagcacccacaacgtgatcaacaccgaggacgcccctggcggcccctacgagatcggcaccagcggcagctgcctgaacatcaccaacggcaagggcttcttcgccacaatggcctgggccgtgcccaagaacaagaccgccaccaatcccctgaccatcgaggtgccctacatctgcaccgaggaggaggaccagatcaccgtgtggggcttccacagcgacgacgagacacagatggccaggctgtacggcgacagcaagccccagaagttcaccagcagcgccaacggcgtgaccacccactacgtgagccagatcggcggcttccccaaccagaccgaggacggcggcctgccccagagcggcaggatcgtggtggactacatggtgcagaagtccggcaagaccggcaccatcacctaccagaggggcatcctgctgccccagaaggtgtggtgcgccagcggcaagagcaaggtgatcaagggcagcctgcccctgatcggcgaggccgactgcctgcacgagaagtacggcggcctgaacaagagcaagccctactacaccggcgagcacgccaaggccatcggcaactgccccatctgggtgaagacccctctgaagctggccaacggcaccaagtacaggcctcccgccaagctgctgaaggagaggggcttcttcggcgccatcgccggcttcctggagggcggctgggagggcatgatcgccggctggcacggctacaccagccacggcgcccacggcgtggccgtggccgccgacctgaagtccacccaggaggccatcaacaagatcaccaagaacctgaacagcctgagcgagctggaggtgaagaacctgcagaggctgagcggcgctatggacgagctgcacaacgagatcctggagctggacgagaaggtggacgacctgagggccgacaccatcagcagccagatcgagctggccgtgctgctgagcaacgagggcatcatcaacagcgaggacgagcacctgctggccctggagaggaagctgaagaagatgctgggccccagcgccgtggagatcggcaacggctgcttcgagacaaagcacaagtgcaaccagacctgcctggacaggatcgccgccggcaccttcgacgccggcgagttcagcctgcccaccttcgacagcctgaacatcaccgccgccagcctgaacgacgacggcctggacaaccacaccatcctgctgtactacagcaccgccgccagcagcctggccgtgaccctgatgatcgccatcttcgtggtgtatatggtgagcagggacaacgtgagctgcagcatctgcctgtgatagtaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttc

SEQ ID NO: 2 - DNA, 11138 bp, synthetic construct
  source 1..11138: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggaccgacagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctcagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctcctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtcctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccacacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgacggagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtaccggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacgcccgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaaccgggtgatcaccagggaggagctggaggccctgacccctagcaggacccctagcaggagcgtgagcaggaccagcctggtgagcaaccctcccggcgtgaaccgggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtcctggagaggacggagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctccaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtcctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaaccgggtgggcatcctgagcgagctgtgcaaggccgtggagagcaggtacgagacagtgggcaccagcatcatcgtgatggccatgaccaccctggccagcagcgtcaagtccttcagctacctgaggggggcccctataactctctacggctaacctgaatggactacgacatagtctagtccgccaaggccgccaccatgaatcctaaccagaagatcatcaccatcggcagcgtgagcctgaccatcagcaccatctgcttcttcatgcagatcgccatcctgatcaccaccgtgaccctgcacttcaagcagtacgagttcaacagccctcccaacaaccaggtgatgctgtgcgagcccaccatcatcgagaggaacatcaccgagatcgtgtacctgaccaacaccaccatcgagaaggagatctgccccaagcccgccgagtacaggaactggagcaagccccagtgcggcatcaccggcttcgcccctttcagcaaggacaacagcatcaggctgagcgccggcggcgacatctgggtgaccagggagccctacgtgagctgcgacctggacaagtgctaccagttcgccctgggccagggcaccaccctgaacaacgtgcacagcaacaacaccgtgagggacaggaccccttaccggaccctgctgatgaacgagctgggcgtgcccttccacctgggcaccaagcaggtgtgcatcgcctggagcagctccagctgccacgacggcaaggcctggctgcacgtgtgcatcaccggcgacgacaagaacgccaccgccagcttcatctacaacggcaggctggtggacagcgtggtgagctggagcaacgacatcctgaggacccaggagagcgagtgcgtgtgcatcaacggcacctgcaccgtggtgatgaccgacggcaacgccaccggcaaggccgacaccaagatcctgttcatcgaggagggcaagatcgtgcacaccagcaagctgagcggcagcgcccagcacgtggaggagtgcagctgctaccccaggtaccccggcgtgaggtgcgtgtgcagggacaactggaagggcagcaaccggcccatcatcgacatcaacatcaaggaccacagcatcgtcagcaggtacgtgtgcagcggcctggtgggcgacacccctagaaagagcgacagcagctccagcagccactgcctgaaccccaacaacgagaagggcgaccacggcgtgaagggctgggccttcgacgacggcaacgacgtgtggatgggcaggaccatcaacgagacaagcaggctgggctacgagacattcaaggtggtggagggctggagcaaccccaagagcaagctgcagatcaacaggcaggtgatcgtggacaggggcgacaggagcggctacagcggcatcttcagcgtggagggcaagagctgcatcaaccggtgcttctacgtggagctgatcaggggcaggaaggaggagacagaggtgctgtggaccagcaacagcatcgtggtgttctgcggcaccagcggcacctacggcaccggcagctggcccgacggcgccaacctgagcctgatgcacatctgatagtaaactcgaagcagtgttaaatcattcagctacctgagaggggcccctataactctctacggctaacctgaatggactacgacatagtctagtccgccaaggccgccaccatgaagaccatcatcgccctgagcaacatcctgtgcctggtgttcgcccagaagatccccggcaatgacaacagcaccgccaccctgtgcctgggccaccacgccgtgcccaacggcaccatcgtgaagaccatcaccaacgacaggatcgaggtgaccaacgccaccgagctggtgcagaacagcagcatcggcgagatctgcggcagccctcaccagatcctggacggcggcaactgcaccctgatcgacgccctgctgggcgaccctcagtgcgacggcttccagaacaaggagtgggacctgttcgtggagaggagcagggccaacagcaactgctacccctacgacgtgcccgactacgccagcctgaggagcctggtggccagcagcggcaccctggagttcaagaacgagagcttcaactggaccggcgtgaagcagaacggcaccagcagcgcctgcatcaggggcagcagctccagcttcttcagccggctgaattggctcaccagcctgaacaacatctaccccgcccagaacgtgaccatgcccaacaaggagcagttcgacaagctgtatatctggggcgtgcaccaccccgacaccgacaagaaccagatcagcctgttcgcccagagcagcggcaggatcaccgtgagcaccaagaggagccagcaggccgtgatccccaacatcggcagcaggcccaggatcaggggcatccccagcaggatcagcatctactggaccatcgtgaagcccggcgacatcctgctgatcaacagcaccggcaacctgatcgcccctaggggctacttcaagatcaggagcggcaagagcagcatcatgaggagcgacgcccctatcggcaagtgcaagagcgagtgcatcacccctaacggcagcatccccaacgacaagcccttccagaacgtgaacaggatcacctacggcgcctgccccaggtacgtgaagcagagcaccctgaagctggccaccggcatgaggaacgtgcccgagaagcagaccaggggcatcttcggcgccatcgccggcttcatcgagaacggctgggagggcatggtggacggctggtacggcttcaggcaccagaacagcgagggcaggggccaggccgccgacctgaagtccacccaggccgccatcgaccagatcaacggcaagctgaacaggctgatcggcaagaccaacgagaagttccaccagatcgagaaggagttcagcgaggtggagggcagggtgcaggacctggagaagtacgtggaggacaccaagatcgacctgtggagctacaacgccgagctgctggtggccctggagaaccagcacaccatcgacctgaccgacagcgagatgaacaagctgttcgagaagaccaagaagcagctgagggagaacgccgaggacatgggcaacggctgcttcaagatctaccacaagtgcgacaacgcctgcatcggcagcatcaggaacgagacatatgaccacaacgtgtaccgggacgaggccctgaacaaccggttccagatcaagggcgtggagctgaagtccggctacaaggactggatcctgtggatcagcttcgccatgagctgcttcctgctgtgcatcgccctgctgggcttcatcatgtgggcctgccagaagggcaacatcaggtgcaacatctgcatctgatagtaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttc

SEQ ID NO: 3 - DNA, 11138 bp, synthetic construct
  source 1..11138: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggaccgacagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctcagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctcctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtcctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccacacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgacggagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtaccggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacgcccgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaaccgggtgatcaccagggaggagctggaggccctgacccctagcaggacccctagcaggagcgtgagcaggaccagcctggtgagcaaccctcccggcgtgaaccgggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtcctggagaggacggagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctccaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtcctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaaccgggtgggcatcctgagcgagctgtgcaaggccgtggagagcaggtacgagacagtgggcaccagcatcatcgtgatggccatgaccaccctggccagcagcgtcaagtccttcagctacctgaggggggcccctataactctctacggctaacctgaatggactacgacatagtctagtccgccaaggccgccaccatgaaccccaaccagaagatcatcaccatcggcagcatctgcatgaccatcggcaccgccaacctgatcctgcagatcggcaacatcatcagcatctgggtgagccacagcatccagatcggcaaccagagccagatcgagacatgcaacaagagcgtgatcacctacgagaacaacacctgggtgaaccagaccttcgtgaacatcagcaacaccaacagcgccgccaggcagagcgtggccagcgtgaagctggccggcaacagcagcctgtgccccgtgagcggctgggccatctacagcaaggacaacagcgtgaggatcggcagcaagggcgacgtgttcgtgatcagggagcccttcatcagctgcagccctctggagtgcaggaccttcttcctgacccagggcgccctgctgaacgacaagcacagcaacggcaccatcaaggacaggagcccctacaggaccctgatgagctgccccatcggcgaggtgcccagcccctacaacagcaggttcgagagcgtggcctggagcgccagcgcctgccacgacggcaccaactggctgaccatcggcatcagcggccccgacagcggcgccgtggccgtgctgaagtacaacggcatcatcaccgacaccatcaagagctggaggaacaagatcctgaggacccaggagagcgagtgcgcctgcgtgaacggcagctgcttcaccatcatgaccgacggccccagcgacggccaggccagctacaagatcttcaggatcgagaagggcaagatcatcaagagcgtggagatgaaggcccctaactaccactacgaggagtgcagctgctaccccgacagcagcgagatcacctgcgtgtgcagggacaactggcacggcagcaacaggccctgggtgagcttcaaccagaacctggagtaccagatgggctacatctgcagcggcgtgttcggcgacaaccccaggcccaacgacaagaccggcagctgcggccccgtgagcagcaacggcgccaacggcgtgaagggcttcagcttcaagtacggcaacggcgtgtggatcggcaggaccaagagcatcagcagcaggaagggcttcgagatgatctgggaccccaacggctggaccggcaccgacaacaagttcagcaagaagcaggacatcgtgggcatcaacgagtggagcggctacagcggcagcttcgtgcagcaccccgagctgaccggcctgaactgcatcaggccctgcttctgggtggagctgatcaggggcaggcccgaggagaacaccatctggaccagcggcagcagcatcagcttctgcggcgtggacagcgacatcgtgggctggagctggcccgacggcgccgagctgcccttcaccatcgacaagtgatagtaaactcgaagcagtgttaaatcattcagctacctgagaggggcccctataactctctacggctaacctgaatggactacgacatagtctagtccgccaaggccgccaccatgaaggccatcctggtggtgatgctgtataccttcaccaccgctaacgccgacaccctgtgcatcggctaccacgccaacaacagcaccgacaccgtggacaccgtgctggagaagaacgtgaccgtgacccacagcgtgaacctgctggaggacaagcacaacggcaagctgtgcaagctgaggggcgtggcccctctgcacctgggcaagtgcaacatcgccggctggatcctgggcaaccccgagtgcgagagcctgagcaccgccaggagctggagctacatcgtggagacaagcaacagcgacaacggcacctgctaccccggcgacttcatcaactacgaggagctgagggagcagctgagcagcgtgagcagcttcgagaggttcgagatcttccccaagaccagcagctggcccaaccacgacagcgacaacggcgtgaccgccgcctgccctcacgccggcgccaagagcttctacaagaacctgatctggctggtgaagaagggcaagagctaccccaagatcaaccagacctacatcaacgacaagggcaaggaggtgctggtgctgtggggcatccaccatcctcccaccatcgccgaccagcagagcctgtaccagaacgccgacgcctacgtgttcgtgggcaccagcaggtacagcaagaagttcaagcccgagatcgccaccaggcccaaggtgagggaccaggagggcaggatgaactactactggaccctggtggagcccggcgacaagatcaccttcgaggccaccggcaacctggtggcccctaggtacgccttcacaatggagagggacgccggcagcggcatcatcattagcgacacccctgtgcacgactgcaacaccacctgccagacccctgagggcgccatcaacaccagcctgcccttccagaacgtgcaccccatcaccatcggcaagtgccccaagtacgtgaagtccaccaagctgaggctggccaccggcctgaggaacgtgcccagcatccagagcaggggcctgttcggcgccatcgccggcttcatcgagggcggctggaccggcatggtggacggctggtacggctaccaccaccagaacgagcagggcagcggctacgccgccgacctgaagtccacccagaacgccatcgacaagatcaccaacaaggtgaacagcgtgatcgagaagatgaacacccagttcaccgccgtgggcaaggagttcaaccacctggagaagaggatcgagaacctgaacaagaaggtggacgacggcttcctggacatctggacctacaacgccgagctgctggtgctgctggagaacgagaggaccctggactaccacgacagcaacgtgaagaacctgtacgagaaggtgaggaaccagctgaagaacaacgccaaggagatcggcaacggctgcttcgagttctaccacaagtgcgacaacacctgcatggagagcgtgaagaacggcacctacgactaccccaagtacagcgaggaggccaagctgaacagggagaagatcgacggcgtgaagctggacagcaccaggatctaccagatcctggccatctacagcaccgtggccagcagcctggtgctggtggtgagcctgggcgccatcagcttctggatgtgcagcaacggcagcctgcagtgcaggatctgcatctgatagtaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttc

SEQ ID NO: 4 - DNA, 11183 bp, synthetic construct
  source 1..11183: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggaccgacagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctcagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctcctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtcctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccacacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgacggagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtaccggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacgcccgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaaccgggtgatcaccagggaggagctggaggccctgacccctagcaggacccctagcaggagcgtgagcaggaccagcctggtgagcaaccctcccggcgtgaaccgggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtcctggagaggacggagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctccaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtcctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaaccgggtgggcatcctgagcgagctgtgcaaggccgtggagagcaggtacgagacagtgggcaccagcatcatcgtgatggccatgaccaccctggccagcagcgtcaagtccttcagctacctgaggggggcccctataactctctacggctaacctgaatggactacgacatagtctagtccgccaaggccgccaccatgctgcctagcaccatccagaccctgacactgttcctgaccagcggcggcgtgctgctgagcctgtacgtgagcgccagcctgagctacctgctgtactccgacatcctgctgaagttcagcaggaccgaggtgaccgcccctatcatgcccctggactgcgccaacgccagcaacgtgcaggccgtgaacaggagcgccaccaagggcgtgacccctctgctgcccgagcccgagtggacctaccccaggctgagctgccccggcagcaccttccagaaggccctgctgatcagccctcacaggttcggcgagacaaagggcaacagcgcccctctgatcatcagggagcccttcatcgcctgcggccccaaggagtgcaagcacttcgccctgacccactacgccgcccagcccggcggctactacaacggcaccagggaggacaggaacaagctgaggcacctgatcagcgtgaagctgggcaagatccccaccgtggagaacagcatcttccacatggccgcctggagcggcagcgcctgccacgacggcagggagtggacctacatcggcgtggacggccccgacagcaacgccctgctgaagatcaagtacggcgaggcctacaccgacacctaccacagctacgccaagaacatcctgaggacccaggagagcgcctgcaactgcatcggcggcgactgctacctgatgatcaccgacggccccgccagcggcatcagcgagtgcaggttcctgaagatcagggagggcaggatcatcaaggagatcttccccaccggcagggtgaagcacaccgaggagtgcacctgcggcttcgccagcaacaagaccatcgagtgcgcctgcagggacaacagctacaccgccaagaggcccttcgtgaagctgaacgtggagacagacaccgccgagatcaggctgatgtgcaccaagacctacctggacacccctaggcccaacgacggcagcatcaccggcccctgcgagagcgacggcgacgagggcagcggcggcatcaagggcggcttcgtgcaccagaggatggccagcaagatcggcaggtggtacagcaggaccatgagcaagaccaagaggatgggcatgggcctgtacgtgaagtacgacggcgacccctggaccgacagcgaggccctggccctgagcggcgtgatggtgagcatggaggagcccggctggtacagcttcggcttcgagatcaaggacaagaagtgcgacgtgccctgcatcggcatcgagatggtgcacgacggcggcaagaccacctggcacagcgccgccaccgccatctactgcctgatgggcagcggccagctgctgtgggacaccgtgaccggcgtgaacatgaccctgtgatagtaaactcgaagcagtgttaaatcattcagctacctgagaggggcccctataactctctacggctaacctgaatggactacgacatagtctagtccgccaaggccgccaccatgaaggccatcatcgtgctgctgatggtggtgacctctaacgccgacaggatctgcaccggcatcaccagcagcaacagccctcacgtggtgaagaccgccacccagggcgaggtgaacgtgaccggcgtgatccctctgaccaccacacccaccaagagctacttcgccaacctgaagggcaccaggaccaggggcaagctgtgccccgactgcctgaactgcaccgacctggacgtggccctgggcaggcccatgtgcgtgggcaccacacccagcgccaaggccagcatcctgcacgaggtgaggcccgtgaccagcggctgcttccccatcatgcacgacaggaccaagatcaggcagctgcccaacctgctgaggggctacgagaagatcaggctgagcacccagaacgtgatcgacgccgagaaggcccctggcggcccctacaggctgggcaccagcggcagctgccccaacgccaccagcaagatcggcttcttcgccacaatggcctgggccgtgcccaaggacaactacaagaacgccaccaatcccctgaccgtggaggtgccctacatctgcaccgagggcgaggaccagatcaccgtgtggggcttccacagcgacaacaagacccagatgaagtccctgtacggcgacagcaatccccagaagttcaccagcagcgccaacggcgtgaccacccactacgtgagccagatcggcgacttccccgaccagaccgaggacggcggcctgccccagagcggcaggatcgtggtggactacatgatgcagaagcccggcaagaccggcaccatcgtgtaccagaggggcgtgctgctgccccagaaggtgtggtgcgccagcggcaggagcaaggtgatcaagggcagcctgcccctgatcggcgaggccgactgcctgcacgaggagtacggcggcctgaacaagagcaagccctactacaccggcaagcacgccaaggccatcggcaactgccccatctgggtgaagacccctctgaagctggccaacggcaccaagtacaggcctcccgccaagctgctgaaggagaggggcttcttcggcgccatcgccggcttcctggagggcggctgggagggcatgatcgccggctggcacggctacaccagccacggcgcccacggcgtggccgtggccgccgacctgaagtccacccaggaggccatcaacaagatcaccaagaacctgaacagcctgagcgagctggaggtgaagaacctgcagaggctgagcggcgctatggacgagctgcacaacgagatcctggagctggacgagaaggtggacgacctgagggccgacaccatcagcagccagatcgagctggccgtgctgctgagcaacgagggcatcatcaacagcgaggacgagcacctgctggccctggagaggaagctgaagaagatgctgggccccagcgccgtggacatcggcaacggctgcttcgagacaaagcacaagtgcaaccagacctgcctggacaggatcgccgccggcaccttcaacgccggcgagttcagcctgcccaccttcgacagcctgaacatcaccgccgccagcctgaacgacgacggcctggacaaccacaccatcctgctgtactacagcaccgccgccagcagcctggccgtgaccctgatgctggccatcttcatcgtgtatatggtgagcagggacaacgtgagctgcagcatctgcctgtgatagtaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttc

SEQ ID NO: 5 - DNA, 1407 bp, synthetic construct
  source 1..1407: mol_type = other DNA
atgctgcctagcaccatccagaccctgacactgttcctgaccagcggcggcgtgctgctgagcctgtacgtgagcgccagcctgagctacctgctgtactccgacatcctgctgaagttcagccagaccgagatcaccgcccctaccatgcccctggactgcgccaacgccagcaacgtgcaggccgtgaacaggagcgccaccaagggcgccaccctgctgctccccgagcccgagtggacctaccccaggctgagctgccccggcagcaccttccagaaggccctgctgatcagccctcacaggttcggcgagacaaagggcaacagcgcccctctgatcatcagggagcccttcgtggcctgcggccccaacgagtgcaagcacttcgccctgacccactacgccgcccagcccggcggctactacaacggcaccaggggcgacaggaacaagctgaggcacctgatcagcgtgaagctgggcaagatccccaccgtggagaacagcatcttccacatggccgcctggagcggcagcgcctgccacgacggcaaggagtggacctacatcggcgtggacggccccgacaacaacgccctgctgaaggtgaagtacggcgaggcctacaccgacacctaccacagctacgccaacaacatcctgaggacccaggagagcgcctgcaactgcatcggcggcaactgctacctgatgatcaccgacggcagcgccagcggcgtgagcgagtgcaggttcctgaagatcagggagggcaggatcatcaaggagatcttccccaccggcagggtgaagcacaccgaggagtgcacctgcggcttcgccagcaacaagaccatcgagtgcgcctgcagggacaacaggtacaccgccaagaggcccttcgtgaagctgaacgtggagacagacaccgccgagatcaggctgatgtgcaccgacacctacctggacacccctaggcccaacgacggcagcatcaccggcccctgcgagagcgacggcgacgagggcagcggcggcatcaagggcggcttcgtgcaccagaggatgaagtccaagatcggcaggtggtacagcaggaccatgagcaagaccgagaggatgggcatgggcctgtacgtgaagtacggcggcgacccctgggccgacagcgacgccctggtgttcagcggcgtgatgatcagcatgaaggagcccggctggtacagcttcggcttcgagatcaaggacaagaagtgcgacgtgccctgcatcggcatcgagatggtgcacgacggcggcaaggagacatggcacagcgccgccaccgccatctactgcctgatgggcagcggccagctgctgtgggacaccatcaccggcgtggacatggccctgtgatagtaa

SEQ ID NO: 6 - DNA, 1416 bp, synthetic construct
  source 1..1416: mol_type = other DNA
atgaatcctaaccagaagatcatcaccatcggcagcgtgagcctgaccatcagcaccatctgcttcttcatgcagatcgccatcctgatcaccaccgtgaccctgcacttcaagcagtacgagttcaacagccctcccaacaaccaggtgatgctgtgcgagcccaccatcatcgagaggaacatcaccgagatcgtgtacctgaccaacaccaccatcgagaaggagatctgccccaagcccgccgagtacaggaactggagcaagccccagtgcggcatcaccggcttcgcccctttcagcaaggacaacagcatcaggctgagcgccggcggcgacatctgggtgaccagggagccctacgtgagctgcgacctggacaagtgctaccagttcgccctgggccagggcaccaccctgaacaacgtgcacagcaacaacaccgtgagggacaggaccccttaccggaccctgctgatgaacgagctgggcgtgcccttccacctgggcaccaagcaggtgtgcatcgcctggagcagctccagctgccacgacggcaaggcctggctgcacgtgtgcatcaccggcgacgacaagaacgccaccgccagcttcatctacaacggcaggctggtggacagcgtggtgagctggagcaacgacatcctgaggacccaggagagcgagtgcgtgtgcatcaacggcacctgcaccgtggtgatgaccgacggcaacgccaccggcaaggccgacaccaagatcctgttcatcgaggagggcaagatcgtgcacaccagcaagctgagcggcagcgcccagcacgtggaggagtgcagctgctaccccaggtaccccggcgtgaggtgcgtgtgcagggacaactggaagggcagcaaccggcccatcatcgacatcaacatcaaggaccacagcatcgtcagcaggtacgtgtgcagcggcctggtgggcgacacccctagaaagagcgacagcagctccagcagccactgcctgaaccccaacaacgagaagggcgaccacggcgtgaagggctgggccttcgacgacggcaacgacgtgtggatgggcaggaccatcaacgagacaagcaggctgggctacgagacattcaaggtggtggagggctggagcaaccccaagagcaagctgcagatcaacaggcaggtgatcgtggacaggggcgacaggagcggctacagcggcatcttcagcgtggagggcaagagctgcatcaaccggtgcttctacgtggagctgatcaggggcaggaaggaggagacagaggtgctgtggaccagcaacagcatcgtggtgttctgcggcaccagcggcacctacggcaccggcagctggcccgacggcgccaacctgagcctgatgcacatctgatagtaa

SEQ ID NO: 7 - DNA, 1416 bp, synthetic construct
  source 1..1416: mol_type = other DNA
atgaaccccaaccagaagatcatcaccatcggcagcatctgcatgaccatcggcaccgccaacctgatcctgcagatcggcaacatcatcagcatctgggtgagccacagcatccagatcggcaaccagagccagatcgagacatgcaacaagagcgtgatcacctacgagaacaacacctgggtgaaccagaccttcgtgaacatcagcaacaccaacagcgccgccaggcagagcgtggccagcgtgaagctggccggcaacagcagcctgtgccccgtgagcggctgggccatctacagcaaggacaacagcgtgaggatcggcagcaagggcgacgtgttcgtgatcagggagcccttcatcagctgcagccctctggagtgcaggaccttcttcctgacccagggcgccctgctgaacgacaagcacagcaacggcaccatcaaggacaggagcccctacaggaccctgatgagctgccccatcggcgaggtgcccagcccctacaacagcaggttcgagagcgtggcctggagcgccagcgcctgccacgacggcaccaactggctgaccatcggcatcagcggccccgacagcggcgccgtggccgtgctgaagtacaacggcatcatcaccgacaccatcaagagctggaggaacaagatcctgaggacccaggagagcgagtgcgcctgcgtgaacggcagctgcttcaccatcatgaccgacggccccagcgacggccaggccagctacaagatcttcaggatcgagaagggcaagatcatcaagagcgtggagatgaaggcccctaactaccactacgaggagtgcagctgctaccccgacagcagcgagatcacctgcgtgtgcagggacaactggcacggcagcaacaggccctgggtgagcttcaaccagaacctggagtaccagatgggctacatctgcagcggcgtgttcggcgacaaccccaggcccaacgacaagaccggcagctgcggccccgtgagcagcaacggcgccaacggcgtgaagggcttcagcttcaagtacggcaacggcgtgtggatcggcaggaccaagagcatcagcagcaggaagggcttcgagatgatctgggaccccaacggctggaccggcaccgacaacaagttcagcaagaagcaggacatcgtgggcatcaacgagtggagcggctacagcggcagcttcgtgcagcaccccgagctgaccggcctgaactgcatcaggccctgcttctgggtggagctgatcaggggcaggcccgaggagaacaccatctggaccagcggcagcagcatcagcttctgcggcgtggacagcgacatcgtgggctggagctggcccgacggcgccgagctgcccttcaccatcgacaagtgatagtaa

SEQ ID NO: 8 - DNA, 1407 bp, synthetic construct
  source 1..1407: mol_type = other DNA
atgctgcctagcaccatccagaccctgacactgttcctgaccagcggcggcgtgctgctgagcctgtacgtgagcgccagcctgagctacctgctgtactccgacatcctgctgaagttcagcaggaccgaggtgaccgcccctatcatgcccctggactgcgccaacgccagcaacgtgcaggccgtgaacaggagcgccaccaagggcgtgacccctctgctgcccgagcccgagtggacctaccccaggctgagctgccccggcagcaccttccagaaggccctgctgatcagccctcacaggttcggcgagacaaagggcaacagcgcccctctgatcatcagggagcccttcatcgcctgcggccccaaggagtgcaagcacttcgccctgacccactacgccgcccagcccggcggctactacaacggcaccagggaggacaggaacaagctgaggcacctgatcagcgtgaagctgggcaagatccccaccgtggagaacagcatcttccacatggccgcctggagcggcagcgcctgccacgacggcagggagtggacctacatcggcgtggacggccccgacagcaacgccctgctgaagatcaagtacggcgaggcctacaccgacacctaccacagctacgccaagaacatcctgaggacccaggagagcgcctgcaactgcatcggcggcgactgctacctgatgatcaccgacggccccgccagcggcatcagcgagtgcaggttcctgaagatcagggagggcaggatcatcaaggagatcttccccaccggcagggtgaagcacaccgaggagtgcacctgcggcttcgccagcaacaagaccatcgagtgcgcctgcagggacaacagctacaccgccaagaggcccttcgtgaagctgaacgtggagacagacaccgccgagatcaggctgatgtgcaccaagacctacctggacacccctaggcccaacgacggcagcatcaccggcccctgcgagagcgacggcgacgagggcagcggcggcatcaagggcggcttcgtgcaccagaggatggccagcaagatcggcaggtggtacagcaggaccatgagcaagaccaagaggatgggcatgggcctgtacgtgaagtacgacggcgacccctggaccgacagcgaggccctggccctgagcggcgtgatggtgagcatggaggagcccggctggtacagcttcggcttcgagatcaaggacaagaagtgcgacgtgccctgcatcggcatcgagatggtgcacgacggcggcaagaccacctggcacagcgccgccaccgccatctactgcctgatgggcagcggccagctgctgtgggacaccgtgaccggcgtgaacatgaccctgtgatagtaa

SEQ ID NO: 9 - DNA, 1755 bp, synthetic construct
  source 1..1755: mol_type = other DNA
atgaaggccatcatcgtgctgctgatggtggtgacctctaacgccgacaggatctgcaccggcatcaccagcagcaacagccctcacgtggtgaagaccgccacccagggcgaggtgaacgtgaccggcgtgatccctctgaccaccacacccaccaagagccacttcgccaacctgaagggcaccgagacaaggggcaagctgtgccccaagtgcctgaactgcaccgacctggacgtggccctgggcaggcccaagtgcaccggcaagatccccagcgccagggtgagcatcctgcacgaggtgaggcccgtgaccagcggctgcttccccatcatgcacgacaggaccaagatcaggcagctgcccaacctgctgaggggctacgagcacgtgaggctgagcacccacaacgtgatcaacaccgaggacgcccctggcggcccctacgagatcggcaccagcggcagctgcctgaacatcaccaacggcaagggcttcttcgccacaatggcctgggccgtgcccaagaacaagaccgccaccaatcccctgaccatcgaggtgccctacatctgcaccgaggaggaggaccagatcaccgtgtggggcttccacagcgacgacgagacacagatggccaggctgtacggcgacagcaagccccagaagttcaccagcagcgccaacggcgtgaccacccactacgtgagccagatcggcggcttccccaaccagaccgaggacggcggcctgccccagagcggcaggatcgtggtggactacatggtgcagaagtccggcaagaccggcaccatcacctaccagaggggcatcctgctgccccagaaggtgtggtgcgccagcggcaagagcaaggtgatcaagggcagcctgcccctgatcggcgaggccgactgcctgcacgagaagtacggcggcctgaacaagagcaagccctactacaccggcgagcacgccaaggccatcggcaactgccccatctgggtgaagacccctctgaagctggccaacggcaccaagtacaggcctcccgccaagctgctgaaggagaggggcttcttcggcgccatcgccggcttcctggagggcggctgggagggcatgatcgccggctggcacggctacaccagccacggcgcccacggcgtggccgtggccgccgacctgaagtccacccaggaggccatcaacaagatcaccaagaacctgaacagcctgagcgagctggaggtgaagaacctgcagaggctgagcggcgctatggacgagctgcacaacgagatcctggagctggacgagaaggtggacgacctgagggccgacaccatcagcagccagatcgagctggccgtgctgctgagcaacgagggcatcatcaacagcgaggacgagcacctgctggccctggagaggaagctgaagaagatgctgggccccagcgccgtggagatcggcaacggctgcttcgagacaaagcacaagtgcaaccagacctgcctggacaggatcgccgccggcaccttcgacgccggcgagttcagcctgcccaccttcgacagcctgaacatcaccgccgccagcctgaacgacgacggcctggacaaccacaccatcctgctgtactacagcaccgccgccagcagcctggccgtgaccctgatgatcgccatcttcgtggtgtatatggtgagcagggacaacgtgagctgcagcatctgcctgtgatagtaa

SEQ ID NO: 10 - DNA, 1707 bp, synthetic construct
  source 1..1707: mol_type = other DNA
atgaagaccatcatcgccctgagcaacatcctgtgcctggtgttcgcccagaagatccccggcaatgacaacagcaccgccaccctgtgcctgggccaccacgccgtgcccaacggcaccatcgtgaagaccatcaccaacgacaggatcgaggtgaccaacgccaccgagctggtgcagaacagcagcatcggcgagatctgcggcagccctcaccagatcctggacggcggcaactgcaccctgatcgacgccctgctgggcgaccctcagtgcgacggcttccagaacaaggagtgggacctgttcgtggagaggagcagggccaacagcaactgctacccctacgacgtgcccgactacgccagcctgaggagcctggtggccagcagcggcaccctggagttcaagaacgagagcttcaactggaccggcgtgaagcagaacggcaccagcagcgcctgcatcaggggcagcagctccagcttcttcagccggctgaattggctcaccagcctgaacaacatctaccccgcccagaacgtgaccatgcccaacaaggagcagttcgacaagctgtatatctggggcgtgcaccaccccgacaccgacaagaaccagatcagcctgttcgcccagagcagcggcaggatcaccgtgagcaccaagaggagccagcaggccgtgatccccaacatcggcagcaggcccaggatcaggggcatccccagcaggatcagcatctactggaccatcgtgaagcccggcgacatcctgctgatcaacagcaccggcaacctgatcgcccctaggggctacttcaagatcaggagcggcaagagcagcatcatgaggagcgacgcccctatcggcaagtgcaagagcgagtgcatcacccctaacggcagcatccccaacgacaagcccttccagaacgtgaacaggatcacctacggcgcctgccccaggtacgtgaagcagagcaccctgaagctggccaccggcatgaggaacgtgcccgagaagcagaccaggggcatcttcggcgccatcgccggcttcatcgagaacggctgggagggcatggtggacggctggtacggcttcaggcaccagaacagcgagggcaggggccaggccgccgacctgaagtccacccaggccgccatcgaccagatcaacggcaagctgaacaggctgatcggcaagaccaacgagaagttccaccagatcgagaaggagttcagcgaggtggagggcagggtgcaggacctggagaagtacgtggaggacaccaagatcgacctgtggagctacaacgccgagctgctggtggccctggagaaccagcacaccatcgacctgaccgacagcgagatgaacaagctgttcgagaagaccaagaagcagctgagggagaacgccgaggacatgggcaacggctgcttcaagatctaccacaagtgcgacaacgcctgcatcggcagcatcaggaacgagacatatgaccacaacgtgtaccgggacgaggccctgaacaaccggttccagatcaagggcgtggagctgaagtccggctacaaggactggatcctgtggatcagcttcgccatgagctgcttcctgctgtgcatcgccctgctgggcttcatcatgtgggcctgccagaagggcaacatcaggtgcaacatctgcatctgatagtaa

SEQ ID NO: 11 - DNA, 1707 bp, synthetic construct
  source 1..1707: mol_type = other DNA
atgaaggccatcctggtggtgatgctgtataccttcaccaccgctaacgccgacaccctgtgcatcggctaccacgccaacaacagcaccgacaccgtggacaccgtgctggagaagaacgtgaccgtgacccacagcgtgaacctgctggaggacaagcacaacggcaagctgtgcaagctgaggggcgtggcccctctgcacctgggcaagtgcaacatcgccggctggatcctgggcaaccccgagtgcgagagcctgagcaccgccaggagctggagctacatcgtggagacaagcaacagcgacaacggcacctgctaccccggcgacttcatcaactacgaggagctgagggagcagctgagcagcgtgagcagcttcgagaggttcgagatcttccccaagaccagcagctggcccaaccacgacagcgacaacggcgtgaccgccgcctgccctcacgccggcgccaagagcttctacaagaacctgatctggctggtgaagaagggcaagagctaccccaagatcaaccagacctacatcaacgacaagggcaaggaggtgctggtgctgtggggcatccaccatcctcccaccatcgccgaccagcagagcctgtaccagaacgccgacgcctacgtgttcgtgggcaccagcaggtacagcaagaagttcaagcccgagatcgccaccaggcccaaggtgagggaccaggagggcaggatgaactactactggaccctggtggagcccggcgacaagatcaccttcgaggccaccggcaacctggtggcccctaggtacgccttcacaatggagagggacgccggcagcggcatcatcattagcgacacccctgtgcacgactgcaacaccacctgccagacccctgagggcgccatcaacaccagcctgcccttccagaacgtgcaccccatcaccatcggcaagtgccccaagtacgtgaagtccaccaagctgaggctggccaccggcctgaggaacgtgcccagcatccagagcaggggcctgttcggcgccatcgccggcttcatcgagggcggctggaccggcatggtggacggctggtacggctaccaccaccagaacgagcagggcagcggctacgccgccgacctgaagtccacccagaacgccatcgacaagatcaccaacaaggtgaacagcgtgatcgagaagatgaacacccagttcaccgccgtgggcaaggagttcaaccacctggagaagaggatcgagaacctgaacaagaaggtggacgacggcttcctggacatctggacctacaacgccgagctgctggtgctgctggagaacgagaggaccctggactaccacgacagcaacgtgaagaacctgtacgagaaggtgaggaaccagctgaagaacaacgccaaggagatcggcaacggctgcttcgagttctaccacaagtgcgacaacacctgcatggagagcgtgaagaacggcacctacgactaccccaagtacagcgaggaggccaagctgaacagggagaagatcgacggcgtgaagctggacagcaccaggatctaccagatcctggccatctacagcaccgtggccagcagcctggtgctggtggtgagcctgggcgccatcagcttctggatgtgcagcaacggcagcctgcagtgcaggatctgcatctgatagtaa

SEQ ID NO: 12 - DNA, 1761 bp, synthetic construct
  source 1..1761: mol_type = other DNA
atgaaggccatcatcgtgctgctgatggtggtgacctctaacgccgacaggatctgcaccggcatcaccagcagcaacagccctcacgtggtgaagaccgccacccagggcgaggtgaacgtgaccggcgtgatccctctgaccaccacacccaccaagagctacttcgccaacctgaagggcaccaggaccaggggcaagctgtgccccgactgcctgaactgcaccgacctggacgtggccctgggcaggcccatgtgcgtgggcaccacacccagcgccaaggccagcatcctgcacgaggtgaggcccgtgaccagcggctgcttccccatcatgcacgacaggaccaagatcaggcagctgcccaacctgctgaggggctacgagaagatcaggctgagcacccagaacgtgatcgacgccgagaaggcccctggcggcccctacaggctgggcaccagcggcagctgccccaacgccaccagcaagatcggcttcttcgccacaatggcctgggccgtgcccaaggacaactacaagaacgccaccaatcccctgaccgtggaggtgccctacatctgcaccgagggcgaggaccagatcaccgtgtggggcttccacagcgacaacaagacccagatgaagtccctgtacggcgacagcaatccccagaagttcaccagcagcgccaacggcgtgaccacccactacgtgagccagatcggcgacttccccgaccagaccgaggacggcggcctgccccagagcggcaggatcgtggtggactacatgatgcagaagcccggcaagaccggcaccatcgtgtaccagaggggcgtgctgctgccccagaaggtgtggtgcgccagcggcaggagcaaggtgatcaagggcagcctgcccctgatcggcgaggccgactgcctgcacgaggagtacggcggcctgaacaagagcaagccctactacaccggcaagcacgccaaggccatcggcaactgccccatctgggtgaagacccctctgaagctggccaacggcaccaagtacaggcctcccgccaagctgctgaaggagaggggcttcttcggcgccatcgccggcttcctggagggcggctgggagggcatgatcgccggctggcacggctacaccagccacggcgcccacggcgtggccgtggccgccgacctgaagtccacccaggaggccatcaacaagatcaccaagaacctgaacagcctgagcgagctggaggtgaagaacctgcagaggctgagcggcgctatggacgagctgcacaacgagatcctggagctggacgagaaggtggacgacctgagggccgacaccatcagcagccagatcgagctggccgtgctgctgagcaacgagggcatcatcaacagcgaggacgagcacctgctggccctggagaggaagctgaagaagatgctgggccccagcgccgtggacatcggcaacggctgcttcgagacaaagcacaagtgcaaccagacctgcctggacaggatcgccgccggcaccttcaacgccggcgagttcagcctgcccaccttcgacagcctgaacatcaccgccgccagcctgaacgacgacggcctggacaaccacaccatcctgctgtactacagcaccgccgccagcagcctggccgtgaccctgatgctggccatcttcatcgtgtatatggtgagcagggacaacgtgagctgcagcatctgcctgtgatagtaa

SEQ ID NO: 13 - DNA, 7482 bp, synthetic construct
  source 1..7482: mol_type = other DNA
atggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggaccgacagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctcagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctcctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtcctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccacacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgacggagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtaccggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacgcccgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaaccgggtgatcaccagggaggagctggaggccctgacccctagcaggacccctagcaggagcgtgagcaggaccagcctggtgagcaaccctcccggcgtgaaccgggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtcctggagaggacggagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctccaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtcctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaaccgggtgggcatcctgagcgagctgtgcaaggccgtggagagcaggtacgagacagtgggcaccagcatcatcgtgatggccatgaccaccctggccagcagcgtcaagtccttcagctacctgaggggggcccctataactctctacggctaa

SEQ ID NO: 14 - DNA, 44 bp, synthetic construct
  source 1..44: mol_type = other DNA
atgggcggcgcatgagagaagcccagaccaattacctacccaaa

SEQ ID NO: 15 - DNA, 338 bp, synthetic construct
  source 1..338: mol_type = other DNA
actcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttc

SEQ ID NO: 16 - DNA, 130 bp, synthetic construct
  source 1..130: mol_type = other DNA
aaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 17 - RNA, 129 nt, synthetic construct
  source 1..129: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatag

SEQ ID NO: 18 - DNA, 270 bp, synthetic construct
  source 1..270: mol_type = other DNA
actcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 19 - DNA, 170 bp, synthetic construct
  source 1..170: mol_type = other DNA
actcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 20 - DNA, 45 bp, synthetic construct
  source 1..45: mol_type = other DNA
gatgggcggcgcatgagagaagcccagaccaattacctacccaaa

SEQ ID NO: 21 - DNA, 45 bp, synthetic construct
  source 1..45: mol_type = other DNA
gataggcggcgcatgagagaagcccagaccaattacctacccaaa

SEQ ID NO: 22 - RNA, 28 nt, synthetic construct
  source 1..28: mol_type = other RNA
attattacatcaaaacaaaaagccgcca

SEQ ID NO: 23 - RNA, 245 nt, synthetic construct
  source 1..245: mol_type = other RNA
cttaagggggcgctgcctacggaggtggcagccatctccttctcggcatcaagcttaccatggtgccccaggccctgctcttggtcccgctgctggtgttccccctctgcttcggcaagttccccatctacaccatccccgacaagctggggccgtggagccccatcgacatccaccacctgtcctgccccaacaacctcgtggtcgaggacgagggctgcaccaacctgagcgggttctcctac

SEQ ID NO: 24 - RNA, 177 nt, synthetic construct
  source 1..177: mol_type = other RNA
tgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagtgttgggttgcg

SEQ ID NO: 25 - RNA, 89 nt, synthetic construct
  source 1..89: mol_type = other RNA
aattattggttaaagaagtatattagtgctaatttccctccgtttgtcctagcttttctcttctgtcaaccccacacgcctttggcaca

SEQ ID NO: 26 - RNA, 569 nt, synthetic construct
  source 1..569: mol_type = other RNA
ctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataat

SEQ ID NO: 27 - RNA, 21 nt, synthetic construct
  source 1..21: mol_type = other RNA
cacaaagagtaaagaagaaca

SEQ ID NO: 28 - RNA, 21 nt, synthetic construct
  source 1..21: mol_type = other RNA
aacactaaaagtagaagaaaa

SEQ ID NO: 29 - RNA, 21 nt, synthetic construct
  source 1..21: mol_type = other RNA
ctcagaaagataagatcagcc

SEQ ID NO: 30 - RNA, 21 nt, synthetic construct
  source 1..21: mol_type = other RNA
aaccaatcgaaagaaaccaaa

SEQ ID NO: 31 - RNA, 21 nt, synthetic construct
  source 1..21: mol_type = other RNA
ctctaatcaccaggagtaaaa

SEQ ID NO: 32 - RNA, 21 nt, synthetic construct
  source 1..21: mol_type = other RNA
gagagagatcttaacaaaaaa

SEQ ID NO: 33 - RNA, 21 nt, synthetic construct
  source 1..21: mol_type = other RNA
tgtgtaacaacaacaacaaca

SEQ ID NO: 34 - RNA, 21 nt, synthetic construct
  source 1..21: mol_type = other RNA
ccgcagtaggaagagaaagcc

SEQ ID NO: 35 - RNA, 21 nt, synthetic construct
  source 1..21: mol_type = other RNA
aaaaaaaaaagaaatcataaa

SEQ ID NO: 36 - RNA, 21 nt, synthetic construct
  source 1..21: mol_type = other RNA
gagagaagaaagaagaagacg

SEQ ID NO: 37 - RNA, 21 nt, synthetic construct
  source 1..21: mol_type = other RNA
caattaaaaatacttaccaaa

SEQ ID NO: 38 - RNA, 21 nt, synthetic construct
  source 1..21: mol_type = other RNA
gcaaacagagtaagcgaaacg

SEQ ID NO: 39 - RNA, 21 nt, synthetic construct
  source 1..21: mol_type = other RNA
gcgaagaagacgaacgcaaag

SEQ ID NO: 40 - RNA, 21 nt, synthetic construct
  source 1..21: mol_type = other RNA
ttaggactgtattgactggcc

SEQ ID NO: 41 - RNA, 21 nt, synthetic construct
  source 1..21: mol_type = other RNA
atcatcggaattcggaaaaag

SEQ ID NO: 42 - RNA, 21 nt, synthetic construct
  source 1..21: mol_type = other RNA
aaaacaaaagttaaagcagac

SEQ ID NO: 43 - RNA, 21 nt, synthetic construct
  source 1..21: mol_type = other RNA
tttatctcaaataagaaggca

SEQ ID NO: 44 - RNA, 21 nt, synthetic construct
  source 1..21: mol_type = other RNA
ggtggggaggtgagatttctt

SEQ ID NO: 45 - RNA, 21 nt, synthetic construct
  source 1..21: mol_type = other RNA
tgattaggaaactacaaagcc

SEQ ID NO: 46 - RNA, 21 nt, synthetic construct
  source 1..21: mol_type = other RNA
catttttcaatttcataaaac

SEQ ID NO: 47 - RNA, 21 nt, synthetic construct
  source 1..21: mol_type = other RNA
ttacttttaagcccaacaaaa

SEQ ID NO: 48 - RNA, 21 nt, synthetic construct
  source 1..21: mol_type = other RNA
ggcgtgtgtgtgtgttgttga

SEQ ID NO: 49 - RNA, 21 nt, synthetic construct
  source 1..21: mol_type = other RNA
gtggtgaaggggaaggtttag

SEQ ID NO: 50 - RNA, 21 nt, synthetic construct
  source 1..21: mol_type = other RNA
ttgtttttttttggtttggtt

SEQ ID NO: 51 - RNA, 158 nt, synthetic construct
  source 1..158: mol_type = other RNA
ctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacat

SEQ ID NO: 52 - RNA, 166 nt, synthetic construct
  source 1..166: mol_type = other RNA
tgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccat

SEQ ID NO: 53 - RNA, 143 nt, synthetic construct
  source 1..143: mol_type = other RNA
acgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgca

SEQ ID NO: 54 - RNA, 220 nt, synthetic construct
  source 1..220: mol_type = other RNA
tagagcggcaaaccctagctacactccatagctagtttcttttttttttgtttttttttttttttttttttttttttttttttttttttcctttcttttccttctttttttcctcttttcttggtggctccatcttagccctagtcacggctagctgtgaaaggtccgtgagccgcatgactgcagagagtgccgtaactggtctctctgcagatcatgt

SEQ ID NO: 55 - RNA, 170 nt, synthetic construct
  source 1..170: mol_type = other RNA
acacatcacaaccacaaccttctcaggctaccctgagaaaaaaagacatgaagactcaggactcatcttttctgttggtgtaaaatcaacaccctaaggaacacaaatttctttaaacatttgacttcttgtctctgtgctgcaattaataaaaaatggaaagaatctac

SEQ ID NO: 56 - RNA, 110 nt, synthetic construct
  source 1..110: mol_type = other RNA
gctggagcctcggtagccgttcctcctgcccgctgggcctcccaacgggccctcctcccctccttgcaccggcccttcctggtctttgaataaagtctgagtgggcagca

SEQ ID NO: 57 - RNA, 123 nt, synthetic construct
  source 1..123: mol_type = other RNA
tagtgcagtcactggcacaacgcgttgcccggtaagccaatcgggtatacacggtcgtcatactgcagacagggttcttctactttgcaagatagtctagagtagtaaaataaatagtataag

SEQ ID NO: 58 - RNA, 44 nt, synthetic construct
  source 1..44: mol_type = other RNA
gtcagctttcaaactctttgtttcttgtttgttgattgagaata

SEQ ID NO: 59 - RNA, 54 nt, synthetic construct
  source 1..54: mol_type = other RNA
ctctcgcctgagaaaaaaaatccacgaaccaatttctcagcaaccagcagcacg

SEQ ID NO: 60 - RNA, 52 nt, synthetic construct
  source 1..52: mol_type = other RNA
acctgtgagggttcgaaggaagtagcagtgttttttgttcctagaggaagag

SEQ ID NO: 61 - RNA, 71 nt, synthetic construct
  source 1..71: mol_type = other RNA
acacagaaacattcgcaaaaacaaaatcccagtatcaaaattcttctctttttttcatatttcgcaaagac

SEQ ID NO: 62 - RNA, 52 nt, synthetic construct
  source 1..52: mol_type = other RNA
cagaaaaatttgctacattgtttcacaaacttcaaatattattcatttattt

SEQ ID NO: 63-64 - skipped (2 entries)

SEQ ID NO: 65 - RNA, 21 nt, synthetic construct
  source 1..21: mol_type = other RNA
aacttaaaaaaaaaaatcaaa

SEQ ID NO: 66 - RNA, 92 nt, synthetic construct
  source 1..92: mol_type = other RNA
tcaagcttttggaccctcgtacagaagctaatacgactcactatagggaaataagagagaaaagaagagtaagaagaaatataagagccacc

SEQ ID NO: 67 - RNA, 53 nt, synthetic construct
  source 1..53: mol_type = other RNA
cacatttgcttctgacatagttgtgttgactcacaaccccagaaacagacatc

SEQ ID NO: 68 - RNA, 50 nt, synthetic construct
  source 1..50: mol_type = other RNA
acatttgcttctgacacaactgtgttcactagcaacctcaaacagacacc

SEQ ID NO: 69 - RNA, 46 nt, synthetic construct
  source 1..46: mol_type = other RNA
tgcacacagatcacctttcctatcaaccccactagcctctggcaaa

SEQ ID NO: 70 - RNA, 66 nt, synthetic construct
  source 1..66: mol_type = other RNA
cataaaccctggcgcgctcgcgggccggcactcttctggtccccacagactcagagagaacccacc

SEQ ID NO: 71 - RNA, 58 nt, synthetic construct
  source 1..58: mol_type = other RNA
ataaaaagaccagcagatgccccacagcactgctcttccagaggcaagaccaaccaag

SEQ ID NO: 72 - RNA, 177 nt, synthetic construct
  source 1..177: mol_type = other RNA
agacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcacagaagtccactcattcttggcagg

SEQ ID NO: 73 - RNA, 93 nt, synthetic construct
  source 1..93: mol_type = other RNA
agataaaaagccagctccagcaggcgctgctcactcctccccatcctctccctctgtccctctgtccctctgaccctgcactgtcccagcacc

SEQ ID NO: 74 - RNA, 30 nt, synthetic construct
  source 1..30: mol_type = other RNA
tatatccgtggtttcctgctacctccaacc

SEQ ID NO: 75 - RNA, 34 nt, synthetic construct
  source 1..34: mol_type = other RNA
ggcaccaccactgacctgggacagtgaatcgaca

SEQ ID NO: 76 - RNA, 79 nt, synthetic construct
  source 1..79: mol_type = other RNA
attcatgaaaatccactactccagacagacggctttggaatccaccagctacatccagctccctgaggcagagttgaga

SEQ ID NO: 77 - RNA, 116 nt, synthetic construct
  source 1..116: mol_type = other RNA
aatattagagtctcaacccccaataaatataggactggagatgtctgaggctcattctgccctcgagcccaccgggaacgaaagagaagctctatctcccctccaggagcccagct

SEQ ID NO: 78 - RNA, 79 nt, synthetic construct
  source 1..79: mol_type = other RNA
aggatgggaactaggagtggcagcaatcctttctttcagctggagtgctcctcaggagccagccccacccttagaaaag

SEQ ID NO: 79 - RNA, 111 nt, synthetic construct
  source 1..111: mol_type = other RNA
agggggagccctataattggacaagtctgggatccttgagtcctactcagccccagcggaggtgaaggacgtccttccccaggagccgactggccaatcacaggcaggaag

SEQ ID NO: 80 - RNA, 223 nt, synthetic construct
  source 1..223: mol_type = other RNA
agacgggtggggcggggcccaactgtccccagctccttcagccctttctgtccctcccagtgaggccagctgcggtgaagagggtgctctcttgcctggagttccctctgctacggctgccccctcccagccctggcccactaagccagacccagctgtcgccattcccacttctggtcctgccacctcctgagctgccttcccgcctggtctgggtagagtc

SEQ ID NO: 81 - RNA, 137 nt, synthetic construct
  source 1..137: mol_type = other RNA
cagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 82 - RNA, 200 nt, synthetic construct
  source 1..200: mol_type = other RNA
gggagaaagcttaccatggtgccccaggccctgctcttggtcccgctgctggtttccccctctgcttcggcaagttccccatctacaccatccccgacaagctggggccgtggagccccatcgacatccaccacctgtcctgccccaacaacctcgtggtcgaggacgagggctgcaccaacctgagcgggttctcctac

SEQ ID NO: 83 - RNA, 239 nt, synthetic construct
  source 1..239: mol_type = other RNA
ggggcgctgcctacggaggtggcagccatctccttctcggcatcaagcttaccatggtgccccaggccctgctcttggtcccgctgctggtgttccccctctgcttcggcaagttccccatctacaccatccccgacaagctggggccgtggagccccatcgacatccaccacctgtcctgccccaacaacctcgtggtcgaggacgagggctgcaccaacctgagcgggttctcctac

SEQ ID NO: 84 - RNA, 147 nt, synthetic construct
  source 1..147: mol_type = other RNA
gaataaatgtatagggggaaaggcaggagccttggggtcgaggaaaacaggtagggtataaaaagggcacgcaagggaccaagtccagcatcctagagtccagattccaaactgctcagagtcctgtggacagatcactgcttggca

SEQ ID NO: 85 - RNA, 35 nt, synthetic construct
  source 1..35: mol_type = other RNA
gacacttctgattctgacagactcaggaagaaacc

SEQ ID NO: 86 - RNA, 158 nt, synthetic construct
  source 1..158: mol_type = other RNA
tgcaaacacagaaatggaggaggaggggaaggaggaggaggaggagaaggaggaggaggtggtggtggtggtggtgggataaaacccctgaggcataaagggctcggccggagtcagcacagcccagcccttccagagagaggcaagagaggtccacg

SEQ ID NO: 87 - RNA, 189 nt, synthetic construct
  source 1..189: mol_type = other RNA
ctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacagg

SEQ ID NO: 88 - RNA, 212 nt, synthetic construct
  source 1..212: mol_type = other RNA
ataggtaattttagaaatagatctgatttgtatctgagacattttagtgaagtggtgagatataagacataatcagaagacatatctacctgaagactttaaggggagagctccctcccccacctggcctctggacctctcagatttaggggaaagaaccagttttcggagtgatcgtctcagtcagcaccatctctgtaggagcatcggcc

SEQ ID NO: 89 - RNA, 101 nt, synthetic construct
  source 1..101: mol_type = other RNA
agagaggagagccatataaagagccagcggctacagccccagctcgcctctgcccacccctgccccttaccccttcattccttccacctttttccttcact

SEQ ID NO: 90 - RNA, 43 nt, synthetic construct
  source 1..43: mol_type = other RNA
tttaaaaggaaagtggttacagggaggccatgcccatgggttt

SEQ ID NO: 91 - RNA, 34 nt, synthetic construct
  source 1..34: mol_type = other RNA
agtccttagactgcacagcagaacagaaggcatg

SEQ ID NO: 92 - RNA, 102 nt, synthetic construct
  source 1..102: mol_type = other RNA
cccccatatcccccttggctcccattgcttaaatacagactaggacagggctctgtctcctcagcctcggtcaccacccagctctgggacagcaagctgaaa

SEQ ID NO: 93 - RNA, 62 nt, synthetic construct
  source 1..62: mol_type = other RNA
agtcagtcctccttcgcttcagctccagttctcctcatgagccatccctaaacgcagacacc

SEQ ID NO: 94 - RNA, 201 nt, synthetic construct
  source 1..201: mol_type = other RNA
tttcctctgccctgctgtgaagggggagagaacaacccgcctcgtgacagggggctggcacagcccgccctagccctgaggagggggcgggacagggggagtcctataattggaccggtctgggatccgatcccctgctcagaccctggaggctaaggacttgtttcggaaggagctgactggccaatcacaattgcgaag

SEQ ID NO: 95 - RNA, 95 nt, synthetic construct
  source 1..95: mol_type = other RNA
ggccggccaccgggtttgggagcagcccaggctcaccttaaccggagcggtgcggacggtcccgcggcgacagggctaatctcggcaggttcgcg

SEQ ID NO: 96 - RNA, 59 nt, synthetic construct
  source 1..59: mol_type = other RNA
gtcctggactgactcccacaactctgccagtctccagcccctgcccttcagtggtacag

SEQ ID NO: 97 - RNA, 59 nt, synthetic construct
  source 1..59: mol_type = other RNA
tttaagtcaacaccaggaactaggacacagttgtccaggtgctgttggccagtcccaac

SEQ ID NO: 98 - RNA, 81 nt, synthetic construct
  source 1..81: mol_type = other RNA
aaggagctggggagtggagtgtaggcactataacctgaaagacgtggtcctgacaggaggacaattctattccctaccaaa

SEQ ID NO: 99 - RNA, 25 nt, synthetic construct
  source 1..25: mol_type = other RNA
accagccagaagccacagtctcatc

SEQ ID NO: 100 - RNA, 218 nt, synthetic construct
  source 1..218: mol_type = other RNA
aaacagagcaggcaggggccctgattcactggccgctggggccagggttgggggctgggggtgcccacagagcttgactagtgggatttgggggggcagtgggtgcagcgagcccggtccgttgactgccagcctgccggcaggtagacaccggccgtgggtgggggaggcggctagctcagtggccttgggccgcgtggcctggtggcagcggagcc

SEQ ID NO: 101 - RNA, 67 nt, synthetic construct
  source 1..67: mol_type = other RNA
ggacttcagcaggactgctcgaaacatcccacttccagcactgcctgcggtgaaggaaccagcagcc

SEQ ID NO: 102 - RNA, 252 nt, synthetic construct
  source 1..252: mol_type = other RNA
agggcctcgtggggggcgggaaggtactgtcccatataagcctctgctcttggggctcaaccgctcgcacccgctgcgctgcacagggggagaaaaggagcccagggtgtgagccggacaacttctggtcctctccttccatctccttaccggcgtccccacctcaggacttttcccgcaggctgcgaggggacccacagttcgtggccacttgcctcctggggagggcgactctcctcccatccactcaag

SEQ ID NO: 103 - RNA, 253 nt, synthetic construct
  source 1..253: mol_type = other RNA
gggggaaaaaaaaacagccaaaatatgccaaaaagcttctcacaacagctcctcagtagaagcaggggccacttgggaaagccagggcctggacgctaatgttccaggctacatcataggtcccttttcgctcagtgaggccaccatcaccacaccatggccacgtaggcctccagccagggcaacaggacctggaggccacccaagactgcagctggctgccgctgggtccccgggccagctcttggccccg

SEQ ID NO: 104 - RNA, 136 nt, synthetic construct
  source 1..136: mol_type = other RNA
gaaccgcggcgaggaggggggtcggaggcccagacttataaaggctgctggacccgcgctacccgccagaccccgccgcccggatcccccgcgctgcctgtcgccccacgtgaccacactactaagcttggtcgcc

SEQ ID NO: 105 - RNA, 120 nt, synthetic construct
  source 1..120: mol_type = other RNA
agggactcatcaaccaggcctggcctctgagttcaacgcagagctagctgggaaatgttccggatgttggccaaggccagtgtgacgctgggctccagagcggcaggttgggtccggacc

SEQ ID NO: 106 - RNA, 146 nt, synthetic construct
  source 1..146: mol_type = other RNA
gctgcccctgtgctgactgctgacagctgactgacgctcgcagctagcaggtacttctgggttgctagcccagagccctgggccggtgaccctgttttccctacttcccgtctttgaccttgggtgccttccaaccttctgttgcc

SEQ ID NO: 107 - RNA, 219 nt, synthetic construct
  source 1..219: mol_type = other RNA
gggtgctaaaagaatcactagggtggggaggcggtcccagtggggcgggtaggggtgtgtgccaggtggtaccgggtattggctggaggaagggcagcccggggttcggggcggtccctgaatctaaaggccctcggctagtctgatccttgccctaagcatagtcccgttagccaaccccctacccgccgtgggctctgctgcccggtgctcgtcagc

SEQ ID NO: 108 - RNA, 239 nt, synthetic construct
  source 1..239: mol_type = other RNA
aggaggaccttggccagcgggcagaatggcagttggtagaggaagggagcaagggggtgtttcctgggacaggggggcggagacctggagactataggctcccccaggactcaagttcattgagtttctgcagacactgaacggctttcagtcttcccgctgtgactatcacctgtgggctccacctgcctgcacctttagtcagcacctttagccagcacctgcgccagaccccagca

SEQ ID NO: 109 - RNA, 14 nt, synthetic construct
  source 1..14: mol_type = other RNA
aggcgccggtcagg

SEQ ID NO: 110 - RNA, 10 nt, synthetic construct
  source 1..10: mol_type = other RNA
accatcaacc

SEQ ID NO: 111 - RNA, 119 nt, synthetic construct
  source 1..119: mol_type = other RNA
tctgccccaccctgtcctctggaacctctgcgagatttagaggaaagaaccagttttcaggcggattgcctcagatcacactatctccacttgcccagccctgtggaagattagcggcc

SEQ ID NO: 112 - RNA, 142 nt, synthetic construct
  source 1..142: mol_type = other RNA
accccctttcctgctcttgcctgtgaacaatggttaattgttcccaagagagcatctgtcagttgttggcaaaatgataaagacatttgaaaatctgtcttctgacaaataaaaagcatttatttcactgcaatgatgtttt

SEQ ID NO: 113 - RNA, 134 nt, synthetic construct
  source 1..134: mol_type = other RNA
gctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 114 - RNA, 106 nt, synthetic construct
  source 1..106: mol_type = other RNA
tggcatccctgtgacccctccccagtgcctctcctggccctggaagttgccactccagtgcccaccagccttgtcctaataaaattaagttgcatcattttgtctg

SEQ ID NO: 115 - RNA, 80 nt, synthetic construct
  source 1..80: mol_type = other RNA
aatgttcttattctttgcacctcttcctatttttggtttgtgaacagaagtaaaaataaatacaaactacttccatctca

SEQ ID NO: 116 - RNA, 178 nt, synthetic construct
  source 1..178: mol_type = other RNA
ccacacccccattcccccactccagataaagcttcagttatatctcacgtgtctggagttctttgccaagagggagaggctgaaatccccagccgcctcacctgcagctcagctccatcctacttgaaacctcacctgttcccaccgcattttctcctggcgttcgcctgctagtgtg

SEQ ID NO: 117 - RNA, 101 nt, synthetic construct
  source 1..101: mol_type = other RNA
aacctacctgccctgcccccgtcccctcccttccttatttattcctgctgccccagaacataggtcttggaataaaatggctggttcttttgttttccaaa

SEQ ID NO: 118 - RNA, 234 nt, synthetic construct
  source 1..234: mol_type = other RNA
actaagttaaatatttctgcacagtgttcccatggccccttgcatttccttcttaactctctgttacacgtcattgaaactacacttttttggtctgtttttgtgctagactgtaagttccttgggggcagggcctttgtctgtctcatctctgtattcccaaatgcctaacagtacagagccatgactcaataaatacatgttaaatggatgaatgaattcctctgaaactct

SEQ ID NO: 119 - RNA, 171 nt, synthetic construct
  source 1..171: mol_type = other RNA
gcaccccagctggggccaggctgggtcgccctggactgtgtgctcaggagccctgggaggctctggagcccactgtacttgctcttgatgcctggcggggtggggtggggggggtgctgggcccctgcctctctgcaggtccctaataaagctgtgtggcagtctgactcc

SEQ ID NO: 120 - RNA, 110 nt, synthetic construct
  source 1..110: mol_type = other RNA
gattcgtcagtagggttgtaaaggtttttcttttcctgagaaaacaaccttttgttttctcaggttttgctttttggcctttccctagctttaaaaaaaaaaaagcaaaa

SEQ ID NO: 121 - RNA, 74 nt, synthetic construct
  source 1..74: mol_type = other RNA
ggactagttataagactgactagcccgatgggcctcccaacgggccctcctcccctccttgcaccgagattaat

SEQ ID NO: 122 - RNA, 201 nt, synthetic construct
  source 1..201: mol_type = other RNA
ggactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatct

SEQ ID NO: 123 - RNA, 105 nt, synthetic construct
  source 1..105: mol_type = other RNA
ccactcaccagtgtctctgctgcactctcctgtgcctccctgccccctggcaactgccacccctgcgctttgtcctaataaaattaagatgcatcatatcacccg

SEQ ID NO: 124 - RNA, 105 nt, synthetic construct
  source 1..105: mol_type = other RNA
gctgccttctgcggggcttgccttctggccatgcccttcttctctcccttgcacctgtacctcttggtctttgaataaagcctgagtaggaagaaaaaaaaaaaa

SEQ ID NO: 125 - RNA, 163 nt, synthetic construct
  source 1..163: mol_type = other RNA
ttcagggctcactagaaggctgcacatggcagggcaggctgggagccatggaagagggggaagtggaagggttgggctatactctgatgggttctagccctgcactgctcagtcaacaataaaaaaatgtgctttggacccataaaaaaaaaaaaaaaaaaaa

SEQ ID NO: 126 - RNA, 604 nt, synthetic construct
  source 1..604: mol_type = other RNA
gagactcagcccaggaggaccaggatcttgccaaagcagtagcatcccatttgtaccaaaacagtgttcttgctctataaaccgtgttagcagctcaggaagatgccgtgaagcattcttattaaaccacctgctatttcattcaaactgtgtttcttttttatttcctcatttttctcccctgctcctaaaacccaaaatcttctaaagaattctagaaggtatgcgatcaaactttttaaagaaagaaaatactttttgactcatggtttaaaggcatcctttccatcttggggaggtcatgggtgctcctggcaacttgcttgaggaagataggtcagaaagcagagtggaccaaccgttcaatgttttacaagcaaaacatacactaagcatggtctgtagctattaaaagcacacaatctgaagggctgtagatgcacagtagtgttttcccagagcatgttcaaaagccctgggttcaatcacaatactgaaaagtaggccaaaaaacattctgaaaatgaaatatttgggtttttttttataacctttagtgactaaataaagacaaatctaagagactaaaaaaaaaaaaaaaaaa

SEQ ID NO: 127 - RNA, 561 nt, synthetic construct
  source 1..561: mol_type = other RNA
aatattcttaatctttgcaccttttcctactttggtgtttgtgaatagaagtaaaaataaatacgactgccacctcacgagaatggacttttccacttgaagacgagagactggagtacagatgctacaccacttttgggcaagtgaagggggagcagccagccacggtggcacaaacctatatcctggtgcttttgaaggtagaagcagggcggtcaggagttaaggccagttgaggctgggctgcagagtgaaagaccatgtctcaagatggtctttctcctccccaaagtagaaaagaaaaccataaaaacaagaggtaaatatattactatttcatcttagaggatagcaggcatcttgaaagggtagagggaccttaaattctcattattgcccccatactacaaactaaaaaacaaacccgaatcaatctcccataaagacagagattcaaataagagtattaaacgttttatttctcaaaccactcacatgcataatgttcttatacacagtgtcaaaataaagagaaatgcatttttatacaaaaaaaaaaaa

SEQ ID NO: 128 - RNA, 46 nt, synthetic construct
  source 1..46: mol_type = other RNA
ctacagcccagccctctaataaagcttcagttgtatttcacccatc

SEQ ID NO: 129 - RNA, 347 nt, synthetic construct
  source 1..347: mol_type = other RNA
aaagttctgctgcacgaagattcctcctgcggcggggggattgctcctcctctggcttggaaacctagcctagaatcagatacactttctttagagtaaagcacaagctgatgagttacgactttgtgaaatggatagccttgaggggaggcgaaaacaggtcccccaaggctatcagatgtcagtgccaatagactgaaacaagtctgtaaagttagcagtcaggggtgttggttggggccggaagaagagacccactgaaactgtagccccttatcaaaacatatccttgcttgaaagaaaaataccaaggacagaaaatgccataaaatcttgactttgcactc

SEQ ID NO: 130 - RNA, 124 nt, synthetic construct
  source 1..124: mol_type = other RNA
cctagagccacatcctgacctctctacacccctgcagcccctcaaccccattatttattcctgccctccccaccaatgaccttgaaataaagacgattttattttcaaaaaaaaaaaaaaaaaa

SEQ ID NO: 131 - RNA, 380 nt, synthetic construct
  source 1..380: mol_type = other RNA
ccaccctaaaatgtcatccttccttctgaattgggttccttccattaaacacaggctggcctggctcgtgcctgatgctacagcaagtccttgactctgtgggttgtgtgtgtgtgtgtgtgtgtgtgtgtgtgtgtgtgtgtgtgtgtgtgtgtgtgtgtgtgtgtgtctgtgtgtgtgtgtgtctttatgccctgagtttgttgtggacttgagatcatagtatgtcttgatatctcctccagccatgcaaataggttgtgggtagaggactgtggctgagaccacagactctggtccaagaaccatctgctctaaaaaaaataaatctgtcatctctggaaaataaagaggacatgctcaatgactcagggtccagc

SEQ ID NO: 132 - RNA, 217 nt, synthetic construct
  source 1..217: mol_type = other RNA
ctgaagggttagaaagtgggggctctgttttctttgctcggttatccgagaagaaagacaaaacggaagatgaaggtgtcacggatcttgtgaactttttaaaactttcaaggtgctattccattgttctttgtactgtagctaaatgtaactgagatgagttactgctttgaaaaaataaagttttacattttttccaccctttaaaaaaaaaaaa

SEQ ID NO: 133 - RNA, 141 nt, synthetic construct
  source 1..141: mol_type = other RNA
gtatccttctcctgtcctgcaacaacatccatatccagccaggtggccctgtctcaagcacctctctggccctctggtggcccttgcttaataaagattctccgagcacattctgagtctctgtgagtgattcaaaaaaaa

SEQ ID NO: 134 - RNA, 1932 nt, synthetic construct
  source 1..1932: mol_type = other RNA
ggacgcctcaggcaccggagccagaccctcccaagaccacccaggccttcctcaaggactctgcctcagacctcagacaggccaccaacgctgttcatcttcatttccccaaggagacttctttctttgtgccttgatgtttgagagttcttcgagcaaacagtggttttgcaatgtctcacaggccctgtttttgtttttgtttttgttttgttttgttttgttctttttttaaatgcaaccaaagtagagtcaacctgctcggcagatgtacttggattctctgaatcgctattctgtttggagagttcctttgggtcttaagcagccagagtacatggaaatgagattatgtcagatctggagaaacaagcaggtgttgggaaatatgtgacttgacatgataagggctgggaatccagaaatcaatagtgagatccatgaaatcaaaccctgaccagtgtgaaaatgtagccttttggacagtaagcctgcaagtctagtgagaactcagagaaagctgaccattctggtctgaagataggcagcgcatcacaggcaagaatatcgaagtcagtagtaggacaggggtcacatcagataccagctcaaattgcactagctatctagaacagttttctccaggtttgcctgagccttgatgcataccatcgccctctgctggtcgcagcagagataagcaagggctgaaaatggaggcaatcctttcccaaggccctgaaagttgtttttcatggtttcaaactgaatttggctcatttgtaactaactgatcacggtgcctggttacactggctgccaagaaggagcgcatgcaatctgattcagtgctctcttcacatcagtttcctgcctccctccctcatctgcggacagcatcctatctcatcaggcttccctgtgtgtcacaaagtagcagccaccaagcaaatatattccttgaattagcacacctgggtgggccatgtgcgcaccaaggaaacaggtgctatagggagcgccaggccaggcttgtctcttaactgtctcgttcttcagtgagagtgggaaagctgtccggagctcccgcgcaggagcctgggtacccacgcagcgagtcaagggagttttcggagccagagagagaaagatgtgaaggctgtggagtaaggctgaaaccagcctcctgccctatagtcccacactgcagggggtgcgactttaaaacagaacttcaagttgttaacactcacaagcattgcattactgtgaaggaagtagccgcatccataacaggatgtgatggtctacagcttttcctttaaaagctgaaaaggtaccatgtgtgctcgctaggcatataatccagatatgctccagagttctgagattcttccatgaaaggttaactagaagctagaatatttttttatatttttgtaacaattggcttttttcatggggggaggggagtagagggttagtatttatagtcctaacaagtccaaaaatttttataagtgtcttcagattataaataaccctccaaattttgcaatgtttacatgttttttttttaagatgacaaatatgcttgatttgctttttaaataaaagtttagctgttctaagagattaacttcaagtaggatggctggttatgatagtttggattttctacaggttctgttgccatgccttttgggtttcagcatcactcgagtcgcagcatgtgggtggggctgtggaaacctggccaggctggacctggtcagccacacctcagagacattgtttccatttggatgtgagcaggcgcaggcctgcatgctctttcctacttagcatcatcagttcttccgcctccttagcatggttctttgtaacagccatgctgggaagctctgaacaataaaatacttccagagtggt

SEQ ID NO: 135 - RNA, 291 nt, synthetic construct
  source 1..291: mol_type = other RNA
agattgtcgaggcatcggtggggccgtcacccttgtttcttttccttttttaaaaaaaaaaaaaaaacagcttttttttttttgagagatacaattctttccccatttaattcatctccaagcaattttacaatagtgtctatcatgttcaccccataacccatactcattaggacttatgatttaagattcctcctaccctgtcttgcttgccgcacctcatgctaatctagtttttgactcaatagatttgcctactctggctgtctcatataaatcgaatgaattatg

SEQ ID NO: 136 - RNA, 257 nt, synthetic construct
  source 1..257: mol_type = other RNA
ctaggtggaaggccgagcaaaacctctgcttactaaagcttactgaatatggggagagggcttagggtgtttggaaaaactgacagtaatcaaactgggacactacactgaaccacagcttcctgtcgcccctcagcccctcccctttttttgtattattgtgggtaaaattttcctgtctgtggacttctggattttgtgacaatagaccatcactgctgtgacctttgttgaaaataaactcgatacttactttg

SEQ ID NO: 137 - RNA, 282 nt, synthetic construct
  source 1..282: mol_type = other RNA
agaatggcctgagcctccagtgttgagtggagacttttcaccaggactccagcatcatcccttcctatccatacagactcccatgccaaggtctgtgatctgctctccacctgtctcacagagaagtgcaatcccgttctctccagcatgttacctaggataactcatcaagaatcaaagactttctttaaatttctctttgccaacacatggaaattctccattgatttctttcctgtcctgttcaataaatgattacacttgcacttaaaaaaaaaaaaa

SEQ ID NO: 138 - RNA, 509 nt, synthetic construct
  source 1..509: mol_type = other RNA
ctccttggatagcccaacccgtcccaagaaggaagctacggcctgtgaagctgttctatggactttcctgctattcttgtgtaagggaagagaatgagataaagagagagtgaagaaagcagagggggaggtaaatgagagaggctgggaaaggggaaacagaaagcagggccgggggaagagtctaagttagagactcacaaagaaactcaagaggggctgggcagtgcagtcacagtcaggcagctgaggggcagggtgtccctgagggaggcgaggctcaggccttgctcccgtctccccgtagctgcctcctgtctgcatgcattcggtctgcagtactacacagtaggtatgcacatgagcacgtaggacacgtgaatgtgccgcatgcatgtgcgtgcctgtgtgtccatcattggcactgttgctcacttgtgcttcctgtgagcaccctgtcttggtttcaattaaatgagaaacatggtcaaaaaaaaaaaaaaaaaaaa

SEQ ID NO: 139 - RNA, 1098 nt, synthetic construct
  source 1..1098: mol_type = other RNA
ccgtggtgactgcctcccaggagctgggtccccagggcctgcactgcctgcatagggggtgaggagggccgcagccacactgcctggaggatatctgagcctgccatgccacctgacacaggctgctggccttcccagaagtctacgcattcattgacactgctgctcctccatcatcaggaagggatggctctgaggtgtctcagcctgacaagcgagcctcgaggagctggaggacggcccaatctgggcagtattgtggaccaccatccctgctgtttagaataggaaatttaatgcttgggacaggagtggggaagctcgtggtgcccgcacccccccagtcagagcctgcaggccttcaaggatctgtgctgagctctgaggccctagatcaacacagctgcctgctgcctcctgcacctccccaggccattccaccctgcaccagagacccacgtgcctgtttgaggattaccctccccaccacggggatttcctacccagctgttctgctaggctcgggagctgaggggaagccactcggggctctcctaggctttcccctaccaagccatcccttctcccagccccaggactgcacttgcaggccatctgttcccttggatgtgtcttctgatgccagcctggcaacttgcatccactagaaaggccatttcagggctcgggttgtcatccctgttccttaggacctgcaactcatgccaagaccacaccatggacaatccactcctctgcctgtaggcccctgacaacttccttcctgctatgagggagacctgcagaactcagaagtcaaggcctgggcagtgtctagtggagagggtaccaagaccagcagagagaagccacctaagtggcctgggggctagcagccattctgagaaatcctgggtcccgagcagcccagggaaacacagcacacatgactgtctcctcgggcctactgcagggaacctggccttcagccagctcctttgtcatcctggactgtagcctacggccaaccataagtgagcctgtatgtttatttaacttttagtaaagtcagtaaaaagcaaaaaaaaaaaaaaaaaa

SEQ ID NO: 140 - RNA, 201 nt, synthetic construct
  source 1..201: mol_type = other RNA
acatctccagaaggaagagtggacaaaaaaatgtgttgactctttggtgtgagccttttggcttaactgtaactgctagtactttaaccacatggtgaagatgtccatgtgagatttctataccttaggaataaaaacttttcaactatttctcttctcctagtctgctttttttttattaaaaaatacttttttccattt

SEQ ID NO: 141 - RNA, 739 nt, synthetic construct
  source 1..739: mol_type = other RNA
tctttccagccccaccctacaagtgtctctctaccaaggtcaatccacaccccagtgatgttagcagaccctccatctttgagtggtcctttcacccttaagccttttgctctggagccatgttctcagcttcagcacaatttacagcttctccaagcatcgccccgtgggatgttttgagacttctctcctcaatggtgacagttggtcaccctgttctgcttcagggtttcagtactgctcagtgttgtttaagagaatcaaaagttcttatggtttggtctgggatcaatagggaaacacaggtagccaactaggaggaaatgtactgaatgctagtacccaagaccttgagcaggaaagtcacccagacacctctgctttcttttgccatctgacctgcagcactgtcaggacatggcctgtggctgtgtgttcaaacacccctcccacaggactcactttgtcccaacaattcagattgcctagaaatacctttctcttacctgtttgttatttatcaatttttcccagtatttttatacggaaaaaattgtattgaagacactttgtatgcagttgataagaggaattcagtataattatggttggtgattatttttataagcacatgccaacgctttactactgtggaaagacaagtgttttaataaaaagatttacattccatgatgtggacgtcatttctttttttttttaacatcatgtgtttggagag

SEQ ID NO: 142 - RNA, 252 nt, synthetic construct
  source 1..252: mol_type = other RNA
caacgtctaggatgtgaagtttgaagatttctgattagctttcatccggtcttcatctctatttatcttagaagtttagtttcccccacctcccctaccttctctaggtggacattaaaccatcgtccaaagtacatgagagtcactgactctgttcacacaactgtatgtcttactgaaggtccctgaaagatgtttgaggcttgggattccaaacttggtttattaaacatatagtcaccatcttcctat

SEQ ID NO: 143 - RNA, 188 nt, synthetic construct
  source 1..188: mol_type = other RNA
gcccatcaccccacctgggtggctggcattcaggaacctaactgaagtcttctctgcaccccctgccaaccccttcccatctacagtgttagtggtcccggtgccacagagaagagcccagttggaagctatacccgatttaattccagaattagtcaaccatcaattagaatccatccacccccctc

SEQ ID NO: 144 - RNA, 254 nt, synthetic construct
  source 1..254: mol_type = other RNA
gcatcctctcaccagactatgccctcctggaggggctgggaatatagcaagaacgaaaagactgtgcaaggcctagagccagcaaagatgctgatgtagccaggccatgccggaaggagcagggtgaagcttcccctctccctacaaatggaaccttgtggaaacaggatgctaaacaccttctgatggagctgttgcctgcaggccactggtctttgggaattttcaataaagtgcttgcgaggaatctccta

SEQ ID NO: 145 - RNA, 243 nt, synthetic construct
  source 1..243: mol_type = other RNA
agccaagactgtgatacttctcctgtaccctgttgacctcagggagtgctgaccctgtctggtgacttagcaccctcctgtccccagcactgctcctttcagctgctggagctcttggcctggacccctgctggtgacaggacaccctctgaacaatcagaagtggctccaagtggagtgagcagtcatgtcccccatgaataaaaattgtgagcagaggtcgcctacaaaaaaaaaaaaaaa

SEQ ID NO: 146 - RNA, 118 nt, synthetic construct
  source 1..118: mol_type = other RNA
agctccggaagtcacaagacacacccttgccttatgaggatcatgctaccactgcatcagtcaggaatgaataaagctactttgattgtgggaaatgccacagaaaaaaaaaaaaaaa

SEQ ID NO: 147 - RNA, 223 nt, synthetic construct
  source 1..223: mol_type = other RNA
aggccagccttgcccctgccccagagcagagctcaagtgacgctactccattctgcatgttgtacattcctagaaacaaacctaacagcgtggatagtttcacagcttaatgctttgcaatgcccaaggtcacttcatcctcatgctataatgccactgtatcaggtaatatatattttgagtaggtgaaggagaaataaacacatctttcctttataaatta

SEQ ID NO: 148 - RNA, 146 nt, synthetic construct
  source 1..146: mol_type = other RNA
gtttctccggctcccagaagcccatgctcaggcaatggcccctaccctaagaccatcccctaatgcagatattgcatttgggtgcagatgtgggggtcgggcaaacggagtaaacaatacagtctgcattctccaaaaaaaaaaaa

SEQ ID NO: 149 - RNA, 216 nt, synthetic construct
  source 1..216: mol_type = other RNA
gcccccatccacacatggaccacgcaaagtgctggacacatcagtcatctccaactggctgaaaggctgaacctcagggctccacccacgtcatggccacgccccctctattacaagagtccgccttgcctgagtcctccctgctgagtaaagctaccctcccaggtccaaaaaaaaaaaaaaaaaaaaaaaaaaaaaaaaaaaaaaaaaaaaaaa

SEQ ID NO: 150 - DNA, 107 bp, synthetic construct
  source 1..107: mol_type = other DNA
actcgaagcagtgttaaatcattcagctacctgagaggggcccctataactctctacggctaacctgaatggactacgacatagtctagtccgccaaggccgccacc

SEQ ID NO: 151 - DNA, 11846 bp, synthetic construct
  source 1..11846: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggaccgacagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctcagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctcctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtcctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccacacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgacggagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtaccggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacgcccgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaaccgggtgatcaccagggaggagctggaggccctgacccctagcaggacccctagcaggagcgtgagcaggaccagcctggtgagcaaccctcccggcgtgaaccgggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtcctggagaggacggagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctccaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtcctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaaccgggtgggcatcctgagcgagctgtgcaaggccgtggagagcaggtacgagacagtgggcaccagcatcatcgtgatggccatgaccaccctggccagcagcgtcaagtccttcagctacctgaggggggcccctataactctctacggctaacctgaatggactacgacatagtctagtccgccaaggccgccaccatgaatcctaaccagaagatcatcaccatcggcagcgtgagcctgaccatcagcaccatctgcttcttcatgcagatcgccatcctgatcaccaccgtgaccctgcacttcaagcagtacgagttcaacagccctcccaacaaccaggtgatgctgtgcgagcccaccatcatcgagaggaacatgaccgagatcgtgtacctgaccaacaccaccatcgagaaggagatctgccccaagcccgccgagtacaggaactggagcaagccccagtgcggcatcaccggcttcgcccctttcagcaaggacaacagcatcaggctgagcgccggcggcgacatctgggtgaccagggagccctacgtgagctgcgacctggacaagtgctaccagttcgccctgggccagggcaccaccctgaacaacgtgcacagcaacaacaccgtgagggacaggaccccttaccggaccctgctgatgaacgagctgggcgtgcccttccacctgggcaccaagcaggtgtgcatcgcctggagcagctccagctgccacgacggcaaggcctggctgcacgtgtgcatcaccggcgacgacaagaacgccaccgccagcttcatctacaacggcaggctggtggacagcgtggtgagctggagcaacgacatcctgaggacccaggagagcgagtgcgtgtgcatcaacggcacctgcaccgtggtgatgaccgacggcaacgccaccggcaaggccgacaccaagatcctgttcatcgaggagggcaagatcgtgcacaccagcaagctgagcggcagcgcccagcacgtggaggagtgcagctgctaccccaggtaccccggcgtgaggtgcgtgtgcagggacaactggaagggcagcaaccggcccatcatcgacatcaacatcaaggaccacagcatcgtcagcaggtacgtgtgcagcggcctggtgggcgacacccctagaaagagcgacagcagctccagcagccactgcctgaaccccaacaacgagaagggcgaccacggcgtgaagggctgggccttcgacgacggcaacgacgtgtggatgggcaggaccatcaacgagacaagcaggctgggctacgagacattcaaggtggtggagggctggagcaaccccaagagcaagctgcagatcaacaggcaggtgatcgtggacaggggcgacaggagcggctacagcggcatcttcagcgtggagggcaagagctgcatcaaccggtgcttctacgtggagctgatcaggggcaggaaggaggagacagaggtgctgtggaccagcaacagcatcgtggtgttctgcggcaccagcggcacctacggcaccggcagctggcccgacggcgccaacctgagcctgatgcacatctgatagtaaactcgaccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgaagaccatcatcgccctgagctacatcctgtgcctggtgttcgcccagaagatccccggcaatgacaacagcaccgccaccctgtgcctgggccaccacgccgtgcccaacggcaccatcgtgaagaccatcaccaacgacaggatcgaggtgaccaacgccaccgagctggtgcagaacagcagcatcggcgagatctgcgacagccctcaccagatcctggacggcggcaactgcaccctgatcgacgccctgctgggcgaccctcagtgcgacggcttccagaacaaggagtgggacctgttcgtggagaggagcagggccaacagcaactgctacccctacgacgtgcccgactacgccagcctgaggagcctggtggccagcagcggcaccctggagttcaagaacgagagcttcaactggaccggcgtgaagcagaacggcaccagcagcgcctgcatcaggggcagcagctccagcttcttcagccggctgaattggctcacccacctgaactacacctaccccgccctgaacgtgaccatgcccaacaacgagcagttcgacaagctgtatatctggggcgtgcaccaccccagcaccgacaaggaccagatcagcctgttcgcccagcccagcggcaggatcaccgtgagcaccaagaggagccagcaggccgtgatccccaacatcggcagcaggcccaggatcagggacatccccagcaggatcagcatctactggaccatcgtgaagcccggcgacatcctgctgatcaacagcaccggcaacctgatcgcccctaggggctacttcaagatcaggagcggcaagagcagcatcatgaggagcgacgcccctatcggcaagtgcaagagcgagtgcatcacccctaacggcagcatccccaacgacaagcccttccagaacgtgaacaggatcacctacggcgcctgccccaggtacgtgaagcagagcaccctgaagctggccaccggcatgaggaacgtgcccgagaagcagaccaggggcatcttcggcgccatcgccggcttcatcgagaacggctgggagggcatggtggacggctggtacggcttcaggcaccagaacagcgagggcaggggccaggccgccgacctgaagtccacccaggccgccatcgaccagatcaacggcaagctgaacaggctgatcggcaagaccaacgagaagttccaccagatcgagaaggagttcagcgaggtggagggcagggtgcaggacctggagaagtacgtggaggacaccaagatcgacctgtggagctacaacgccgagctgctggtggccctggagaaccagcacaccatcgacctgaccgacagcgagatgaacaagctgttcgagaagaccaagaagcagctgagggagaacgccgaggacatgggcaacggctgcttcaagatctaccacaagtgcgacaacgcctgcatcggcagcatcaggaacgagacatatgaccacaacgtgtaccgggacgaggccctgaacaaccggttccagatcaagggcgtggagctgaagtccggctacaaggactggatcctgtggatcagcttcgccatgagctgcttcctgctgtgcatcgccctgctgggcttcatcatgtgggcctgccagaagggcaacatcaggtgcaacatctgcatctgatagtaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttc

SEQ ID NO: 152 - DNA, 12415 bp, synthetic construct
  source 1..12415: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggaccgacagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctcagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctcctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtcctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccacacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgacggagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtaccggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacgcccgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaaccgggtgatcaccagggaggagctggaggccctgacccctagcaggacccctagcaggagcgtgagcaggaccagcctggtgagcaaccctcccggcgtgaaccgggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtcctggagaggacggagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctccaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtcctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaaccgggtgggcatcctgagcgagctgtgcaaggccgtggagagcaggtacgagacagtgggcaccagcatcatcgtgatggccatgaccaccctggccagcagcgtcaagtccttcagctacctgaggggggcccctataactctctacggctaacctgaatggactacgacatagtctagtccgccaaggcccttaagctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataatatgaatcctaaccagaagatcatcaccatcggcagcgtgagcctgaccatcagcaccatctgcttcttcatgcagatcgccatcctgatcaccaccgtgaccctgcacttcaagcagtacgagttcaacagccctcccaacaaccaggtgatgctgtgcgagcccaccatcatcgagaggaacatgaccgagatcgtgtacctgaccaacaccaccatcgagaaggagatctgccccaagcccgccgagtacaggaactggagcaagccccagtgcggcatcaccggcttcgcccctttcagcaaggacaacagcatcaggctgagcgccggcggcgacatctgggtgaccagggagccctacgtgagctgcgacctggacaagtgctaccagttcgccctgggccagggcaccaccctgaacaacgtgcacagcaacaacaccgtgagggacaggaccccttaccggaccctgctgatgaacgagctgggcgtgcccttccacctgggcaccaagcaggtgtgcatcgcctggagcagctccagctgccacgacggcaaggcctggctgcacgtgtgcatcaccggcgacgacaagaacgccaccgccagcttcatctacaacggcaggctggtggacagcgtggtgagctggagcaacgacatcctgaggacccaggagagcgagtgcgtgtgcatcaacggcacctgcaccgtggtgatgaccgacggcaacgccaccggcaaggccgacaccaagatcctgttcatcgaggagggcaagatcgtgcacaccagcaagctgagcggcagcgcccagcacgtggaggagtgcagctgctaccccaggtaccccggcgtgaggtgcgtgtgcagggacaactggaagggcagcaaccggcccatcatcgacatcaacatcaaggaccacagcatcgtcagcaggtacgtgtgcagcggcctggtgggcgacacccctagaaagagcgacagcagctccagcagccactgcctgaaccccaacaacgagaagggcgaccacggcgtgaagggctgggccttcgacgacggcaacgacgtgtggatgggcaggaccatcaacgagacaagcaggctgggctacgagacattcaaggtggtggagggctggagcaaccccaagagcaagctgcagatcaacaggcaggtgatcgtggacaggggcgacaggagcggctacagcggcatcttcagcgtggagggcaagagctgcatcaaccggtgcttctacgtggagctgatcaggggcaggaaggaggagacagaggtgctgtggaccagcaacagcatcgtggtgttctgcggcaccagcggcacctacggcaccggcagctggcccgacggcgccaacctgagcctgatgcacatctgatagtaaactcgaccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgaagaccatcatcgccctgagctacatcctgtgcctggtgttcgcccagaagatccccggcaatgacaacagcaccgccaccctgtgcctgggccaccacgccgtgcccaacggcaccatcgtgaagaccatcaccaacgacaggatcgaggtgaccaacgccaccgagctggtgcagaacagcagcatcggcgagatctgcgacagccctcaccagatcctggacggcggcaactgcaccctgatcgacgccctgctgggcgaccctcagtgcgacggcttccagaacaaggagtgggacctgttcgtggagaggagcagggccaacagcaactgctacccctacgacgtgcccgactacgccagcctgaggagcctggtggccagcagcggcaccctggagttcaagaacgagagcttcaactggaccggcgtgaagcagaacggcaccagcagcgcctgcatcaggggcagcagctccagcttcttcagccggctgaattggctcacccacctgaactacacctaccccgccctgaacgtgaccatgcccaacaacgagcagttcgacaagctgtatatctggggcgtgcaccaccccagcaccgacaaggaccagatcagcctgttcgcccagcccagcggcaggatcaccgtgagcaccaagaggagccagcaggccgtgatccccaacatcggcagcaggcccaggatcagggacatccccagcaggatcagcatctactggaccatcgtgaagcccggcgacatcctgctgatcaacagcaccggcaacctgatcgcccctaggggctacttcaagatcaggagcggcaagagcagcatcatgaggagcgacgcccctatcggcaagtgcaagagcgagtgcatcacccctaacggcagcatccccaacgacaagcccttccagaacgtgaacaggatcacctacggcgcctgccccaggtacgtgaagcagagcaccctgaagctggccaccggcatgaggaacgtgcccgagaagcagaccaggggcatcttcggcgccatcgccggcttcatcgagaacggctgggagggcatggtggacggctggtacggcttcaggcaccagaacagcgagggcaggggccaggccgccgacctgaagtccacccaggccgccatcgaccagatcaacggcaagctgaacaggctgatcggcaagaccaacgagaagttccaccagatcgagaaggagttcagcgaggtggagggcagggtgcaggacctggagaagtacgtggaggacaccaagatcgacctgtggagctacaacgccgagctgctggtggccctggagaaccagcacaccatcgacctgaccgacagcgagatgaacaagctgttcgagaagaccaagaagcagctgagggagaacgccgaggacatgggcaacggctgcttcaagatctaccacaagtgcgacaacgcctgcatcggcagcatcaggaacgagacatatgaccacaacgtgtaccgggacgaggccctgaacaaccggttccagatcaagggcgtggagctgaagtccggctacaaggactggatcctgtggatcagcttcgccatgagctgcttcctgctgtgcatcgccctgctgggcttcatcatgtgggcctgccagaagggcaacatcaggtgcaacatctgcatctgatagtaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttc

SEQ ID NO: 153 - DNA, 12448 bp, synthetic construct
  source 1..12448: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggaccgacagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctcagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctcctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtcctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccacacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgacggagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtaccggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacgcccgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaaccgggtgatcaccagggaggagctggaggccctgacccctagcaggacccctagcaggagcgtgagcaggaccagcctggtgagcaaccctcccggcgtgaaccgggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtcctggagaggacggagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctccaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtcctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaaccgggtgggcatcctgagcgagctgtgcaaggccgtggagagcaggtacgagacagtgggcaccagcatcatcgtgatggccatgaccaccctggccagcagcgtcaagtccttcagctacctgaggggggcccctataactctctacggctaacctgaatggactacgacatagtctagtccgccaaggcccttaagttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgaatcctaaccagaagatcatcaccatcggcagcgtgagcctgaccatcagcaccatctgcttcttcatgcagatcgccatcctgatcaccaccgtgaccctgcacttcaagcagtacgagttcaacagccctcccaacaaccaggtgatgctgtgcgagcccaccatcatcgagaggaacatgaccgagatcgtgtacctgaccaacaccaccatcgagaaggagatctgccccaagcccgccgagtacaggaactggagcaagccccagtgcggcatcaccggcttcgcccctttcagcaaggacaacagcatcaggctgagcgccggcggcgacatctgggtgaccagggagccctacgtgagctgcgacctggacaagtgctaccagttcgccctgggccagggcaccaccctgaacaacgtgcacagcaacaacaccgtgagggacaggaccccttaccggaccctgctgatgaacgagctgggcgtgcccttccacctgggcaccaagcaggtgtgcatcgcctggagcagctccagctgccacgacggcaaggcctggctgcacgtgtgcatcaccggcgacgacaagaacgccaccgccagcttcatctacaacggcaggctggtggacagcgtggtgagctggagcaacgacatcctgaggacccaggagagcgagtgcgtgtgcatcaacggcacctgcaccgtggtgatgaccgacggcaacgccaccggcaaggccgacaccaagatcctgttcatcgaggagggcaagatcgtgcacaccagcaagctgagcggcagcgcccagcacgtggaggagtgcagctgctaccccaggtaccccggcgtgaggtgcgtgtgcagggacaactggaagggcagcaaccggcccatcatcgacatcaacatcaaggaccacagcatcgtcagcaggtacgtgtgcagcggcctggtgggcgacacccctagaaagagcgacagcagctccagcagccactgcctgaaccccaacaacgagaagggcgaccacggcgtgaagggctgggccttcgacgacggcaacgacgtgtggatgggcaggaccatcaacgagacaagcaggctgggctacgagacattcaaggtggtggagggctggagcaaccccaagagcaagctgcagatcaacaggcaggtgatcgtggacaggggcgacaggagcggctacagcggcatcttcagcgtggagggcaagagctgcatcaaccggtgcttctacgtggagctgatcaggggcaggaaggaggagacagaggtgctgtggaccagcaacagcatcgtggtgttctgcggcaccagcggcacctacggcaccggcagctggcccgacggcgccaacctgagcctgatgcacatctgatagtaaactcgaagcagtgttaaatcattcagctacctgagaggggcccctataactctctacggctaacctgaatggactacgacatagtctagtccgccaaggccgaattcctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataatatgaagaccatcatcgccctgagctacatcctgtgcctggtgttcgcccagaagatccccggcaatgacaacagcaccgccaccctgtgcctgggccaccacgccgtgcccaacggcaccatcgtgaagaccatcaccaacgacaggatcgaggtgaccaacgccaccgagctggtgcagaacagcagcatcggcgagatctgcgacagccctcaccagatcctggacggcggcaactgcaccctgatcgacgccctgctgggcgaccctcagtgcgacggcttccagaacaaggagtgggacctgttcgtggagaggagcagggccaacagcaactgctacccctacgacgtgcccgactacgccagcctgaggagcctggtggccagcagcggcaccctggagttcaagaacgagagcttcaactggaccggcgtgaagcagaacggcaccagcagcgcctgcatcaggggcagcagctccagcttcttcagccggctgaattggctcacccacctgaactacacctaccccgccctgaacgtgaccatgcccaacaacgagcagttcgacaagctgtatatctggggcgtgcaccaccccagcaccgacaaggaccagatcagcctgttcgcccagcccagcggcaggatcaccgtgagcaccaagaggagccagcaggccgtgatccccaacatcggcagcaggcccaggatcagggacatccccagcaggatcagcatctactggaccatcgtgaagcccggcgacatcctgctgatcaacagcaccggcaacctgatcgcccctaggggctacttcaagatcaggagcggcaagagcagcatcatgaggagcgacgcccctatcggcaagtgcaagagcgagtgcatcacccctaacggcagcatccccaacgacaagcccttccagaacgtgaacaggatcacctacggcgcctgccccaggtacgtgaagcagagcaccctgaagctggccaccggcatgaggaacgtgcccgagaagcagaccaggggcatcttcggcgccatcgccggcttcatcgagaacggctgggagggcatggtggacggctggtacggcttcaggcaccagaacagcgagggcaggggccaggccgccgacctgaagtccacccaggccgccatcgaccagatcaacggcaagctgaacaggctgatcggcaagaccaacgagaagttccaccagatcgagaaggagttcagcgaggtggagggcagggtgcaggacctggagaagtacgtggaggacaccaagatcgacctgtggagctacaacgccgagctgctggtggccctggagaaccagcacaccatcgacctgaccgacagcgagatgaacaagctgttcgagaagaccaagaagcagctgagggagaacgccgaggacatgggcaacggctgcttcaagatctaccacaagtgcgacaacgcctgcatcggcagcatcaggaacgagacatatgaccacaacgtgtaccgggacgaggccctgaacaaccggttccagatcaagggcgtggagctgaagtccggctacaaggactggatcctgtggatcagcttcgccatgagctgcttcctgctgtgcatcgccctgctgggcttcatcatgtgggcctgccagaagggcaacatcaggtgcaacatctgcatctgatagtaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttc

SEQ ID NO: 154 - DNA, 12276 bp, synthetic construct
  source 1..12276: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggaccgacagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctcagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctcctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtcctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccacacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgacggagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtaccggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacgcccgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaaccgggtgatcaccagggaggagctggaggccctgacccctagcaggacccctagcaggagcgtgagcaggaccagcctggtgagcaaccctcccggcgtgaaccgggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtcctggagaggacggagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctccaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtcctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaaccgggtgggcatcctgagcgagctgtgcaaggccgtggagagcaggtacgagacagtgggcaccagcatcatcgtgatggccatgaccaccctggccagcagcgtcaagtccttcagctacctgaggggggcccctataactctctacggctaacctgaatggactacgacatagtctagtccgccaaggcccttaagctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataatatgaatcctaaccagaagatcatcaccatcggcagcgtgagcctgaccatcagcaccatctgcttcttcatgcagatcgccatcctgatcaccaccgtgaccctgcacttcaagcagtacgagttcaacagccctcccaacaaccaggtgatgctgtgcgagcccaccatcatcgagaggaacatgaccgagatcgtgtacctgaccaacaccaccatcgagaaggagatctgccccaagcccgccgagtacaggaactggagcaagccccagtgcggcatcaccggcttcgcccctttcagcaaggacaacagcatcaggctgagcgccggcggcgacatctgggtgaccagggagccctacgtgagctgcgacctggacaagtgctaccagttcgccctgggccagggcaccaccctgaacaacgtgcacagcaacaacaccgtgagggacaggaccccttaccggaccctgctgatgaacgagctgggcgtgcccttccacctgggcaccaagcaggtgtgcatcgcctggagcagctccagctgccacgacggcaaggcctggctgcacgtgtgcatcaccggcgacgacaagaacgccaccgccagcttcatctacaacggcaggctggtggacagcgtggtgagctggagcaacgacatcctgaggacccaggagagcgagtgcgtgtgcatcaacggcacctgcaccgtggtgatgaccgacggcaacgccaccggcaaggccgacaccaagatcctgttcatcgaggagggcaagatcgtgcacaccagcaagctgagcggcagcgcccagcacgtggaggagtgcagctgctaccccaggtaccccggcgtgaggtgcgtgtgcagggacaactggaagggcagcaaccggcccatcatcgacatcaacatcaaggaccacagcatcgtcagcaggtacgtgtgcagcggcctggtgggcgacacccctagaaagagcgacagcagctccagcagccactgcctgaaccccaacaacgagaagggcgaccacggcgtgaagggctgggccttcgacgacggcaacgacgtgtggatgggcaggaccatcaacgagacaagcaggctgggctacgagacattcaaggtggtggagggctggagcaaccccaagagcaagctgcagatcaacaggcaggtgatcgtggacaggggcgacaggagcggctacagcggcatcttcagcgtggagggcaagagctgcatcaaccggtgcttctacgtggagctgatcaggggcaggaaggaggagacagaggtgctgtggaccagcaacagcatcgtggtgttctgcggcaccagcggcacctacggcaccggcagctggcccgacggcgccaacctgagcctgatgcacatctgatagtaaactcgaagcagtgttaaatcattcagctacctgagaggggcccctataactctctacggctaacctgaatggactacgacatagtctagtccgccaaggccgaattcctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataatatgaagaccatcatcgccctgagctacatcctgtgcctggtgttcgcccagaagatccccggcaatgacaacagcaccgccaccctgtgcctgggccaccacgccgtgcccaacggcaccatcgtgaagaccatcaccaacgacaggatcgaggtgaccaacgccaccgagctggtgcagaacagcagcatcggcgagatctgcgacagccctcaccagatcctggacggcggcaactgcaccctgatcgacgccctgctgggcgaccctcagtgcgacggcttccagaacaaggagtgggacctgttcgtggagaggagcagggccaacagcaactgctacccctacgacgtgcccgactacgccagcctgaggagcctggtggccagcagcggcaccctggagttcaagaacgagagcttcaactggaccggcgtgaagcagaacggcaccagcagcgcctgcatcaggggcagcagctccagcttcttcagccggctgaattggctcacccacctgaactacacctaccccgccctgaacgtgaccatgcccaacaacgagcagttcgacaagctgtatatctggggcgtgcaccaccccagcaccgacaaggaccagatcagcctgttcgcccagcccagcggcaggatcaccgtgagcaccaagaggagccagcaggccgtgatccccaacatcggcagcaggcccaggatcagggacatccccagcaggatcagcatctactggaccatcgtgaagcccggcgacatcctgctgatcaacagcaccggcaacctgatcgcccctaggggctacttcaagatcaggagcggcaagagcagcatcatgaggagcgacgcccctatcggcaagtgcaagagcgagtgcatcacccctaacggcagcatccccaacgacaagcccttccagaacgtgaacaggatcacctacggcgcctgccccaggtacgtgaagcagagcaccctgaagctggccaccggcatgaggaacgtgcccgagaagcagaccaggggcatcttcggcgccatcgccggcttcatcgagaacggctgggagggcatggtggacggctggtacggcttcaggcaccagaacagcgagggcaggggccaggccgccgacctgaagtccacccaggccgccatcgaccagatcaacggcaagctgaacaggctgatcggcaagaccaacgagaagttccaccagatcgagaaggagttcagcgaggtggagggcagggtgcaggacctggagaagtacgtggaggacaccaagatcgacctgtggagctacaacgccgagctgctggtggccctggagaaccagcacaccatcgacctgaccgacagcgagatgaacaagctgttcgagaagaccaagaagcagctgagggagaacgccgaggacatgggcaacggctgcttcaagatctaccacaagtgcgacaacgcctgcatcggcagcatcaggaacgagacatatgaccacaacgtgtaccgggacgaggccctgaacaaccggttccagatcaagggcgtggagctgaagtccggctacaaggactggatcctgtggatcagcttcgccatgagctgcttcctgctgtgcatcgccctgctgggcttcatcatgtgggcctgccagaagggcaacatcaggtgcaacatctgcatctgatagtaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttc

SEQ ID NO: 155 - DNA, 12450 bp, synthetic construct
  source 1..12450: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggaccgacagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctcagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctcctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtcctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccacacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgacggagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtaccggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacgcccgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaaccgggtgatcaccagggaggagctggaggccctgacccctagcaggacccctagcaggagcgtgagcaggaccagcctggtgagcaaccctcccggcgtgaaccgggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtcctggagaggacggagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctccaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtcctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaaccgggtgggcatcctgagcgagctgtgcaaggccgtggagagcaggtacgagacagtgggcaccagcatcatcgtgatggccatgaccaccctggccagcagcgtcaagtccttcagctacctgaggggggcccctataactctctacggctaacctgaatggactacgacatagtctagtccgccaaggcccttaagctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataatatgaatcctaaccagaagatcatcaccatcggcagcgtgagcctgaccatcagcaccatctgcttcttcatgcagatcgccatcctgatcaccaccgtgaccctgcacttcaagcagtacgagttcaacagccctcccaacaaccaggtgatgctgtgcgagcccaccatcatcgagaggaacatgaccgagatcgtgtacctgaccaacaccaccatcgagaaggagatctgccccaagcccgccgagtacaggaactggagcaagccccagtgcggcatcaccggcttcgcccctttcagcaaggacaacagcatcaggctgagcgccggcggcgacatctgggtgaccagggagccctacgtgagctgcgacctggacaagtgctaccagttcgccctgggccagggcaccaccctgaacaacgtgcacagcaacaacaccgtgagggacaggaccccttaccggaccctgctgatgaacgagctgggcgtgcccttccacctgggcaccaagcaggtgtgcatcgcctggagcagctccagctgccacgacggcaaggcctggctgcacgtgtgcatcaccggcgacgacaagaacgccaccgccagcttcatctacaacggcaggctggtggacagcgtggtgagctggagcaacgacatcctgaggacccaggagagcgagtgcgtgtgcatcaacggcacctgcaccgtggtgatgaccgacggcaacgccaccggcaaggccgacaccaagatcctgttcatcgaggagggcaagatcgtgcacaccagcaagctgagcggcagcgcccagcacgtggaggagtgcagctgctaccccaggtaccccggcgtgaggtgcgtgtgcagggacaactggaagggcagcaaccggcccatcatcgacatcaacatcaaggaccacagcatcgtcagcaggtacgtgtgcagcggcctggtgggcgacacccctagaaagagcgacagcagctccagcagccactgcctgaaccccaacaacgagaagggcgaccacggcgtgaagggctgggccttcgacgacggcaacgacgtgtggatgggcaggaccatcaacgagacaagcaggctgggctacgagacattcaaggtggtggagggctggagcaaccccaagagcaagctgcagatcaacaggcaggtgatcgtggacaggggcgacaggagcggctacagcggcatcttcagcgtggagggcaagagctgcatcaaccggtgcttctacgtggagctgatcaggggcaggaaggaggagacagaggtgctgtggaccagcaacagcatcgtggtgttctgcggcaccagcggcacctacggcaccggcagctggcccgacggcgccaacctgagcctgatgcacatctgatagtaaactcgaagcagtgttaaatcattcagctacctgagaggggcccctataactctctacggctaacctgaatggactacgacatagtctagtccgccaaggccgaattcttaaaacagctgtgggttgtcacccacccacagggtccactgggcgctagtacactggtatctcggtacctttgtacgcctgttttataccccctccctgatttgcaacttagaagcaacgcaaaccagatcaatagtaggtgtgacataccagtcgcatcttgatcaagcacttctgtatccccggaccgagtatcaatagactgtgcacacggttgaaggagaaaacgtccgttacccggctaactacttcgagaagcctagtaacgccattgaagttgcagagtgtttcgctcagcactccccccgtgtagatcaggtcgatgagtcaccgcattccccacgggcgaccgtggcggtggctgcgttggcggcctgcctatggggtaacccataggacgctctaatacggacatggcgtgaagagtctattgagctagttagtagtcctccggcccctgaatgcggctaatcctaactgcggagcacatacccttaatccaaagggcagtgtgtcgtaacgggcaactctgcagcggaaccgactactttgggtgtccgtgtttctttttattcttgtattggctgcttatggtgacaattaaagaattgttaccatatagctattggattggccatccagtgtcaaacagagctattgtatatctctttgttggattcacacctctcactcttgaaacgttacacaccctcaattacattatactgctgaacacgaagcgatgaagaccatcatcgccctgagctacatcctgtgcctggtgttcgcccagaagatccccggcaatgacaacagcaccgccaccctgtgcctgggccaccacgccgtgcccaacggcaccatcgtgaagaccatcaccaacgacaggatcgaggtgaccaacgccaccgagctggtgcagaacagcagcatcggcgagatctgcgacagccctcaccagatcctggacggcggcaactgcaccctgatcgacgccctgctgggcgaccctcagtgcgacggcttccagaacaaggagtgggacctgttcgtggagaggagcagggccaacagcaactgctacccctacgacgtgcccgactacgccagcctgaggagcctggtggccagcagcggcaccctggagttcaagaacgagagcttcaactggaccggcgtgaagcagaacggcaccagcagcgcctgcatcaggggcagcagctccagcttcttcagccggctgaattggctcacccacctgaactacacctaccccgccctgaacgtgaccatgcccaacaacgagcagttcgacaagctgtatatctggggcgtgcaccaccccagcaccgacaaggaccagatcagcctgttcgcccagcccagcggcaggatcaccgtgagcaccaagaggagccagcaggccgtgatccccaacatcggcagcaggcccaggatcagggacatccccagcaggatcagcatctactggaccatcgtgaagcccggcgacatcctgctgatcaacagcaccggcaacctgatcgcccctaggggctacttcaagatcaggagcggcaagagcagcatcatgaggagcgacgcccctatcggcaagtgcaagagcgagtgcatcacccctaacggcagcatccccaacgacaagcccttccagaacgtgaacaggatcacctacggcgcctgccccaggtacgtgaagcagagcaccctgaagctggccaccggcatgaggaacgtgcccgagaagcagaccaggggcatcttcggcgccatcgccggcttcatcgagaacggctgggagggcatggtggacggctggtacggcttcaggcaccagaacagcgagggcaggggccaggccgccgacctgaagtccacccaggccgccatcgaccagatcaacggcaagctgaacaggctgatcggcaagaccaacgagaagttccaccagatcgagaaggagttcagcgaggtggagggcagggtgcaggacctggagaagtacgtggaggacaccaagatcgacctgtggagctacaacgccgagctgctggtggccctggagaaccagcacaccatcgacctgaccgacagcgagatgaacaagctgttcgagaagaccaagaagcagctgagggagaacgccgaggacatgggcaacggctgcttcaagatctaccacaagtgcgacaacgcctgcatcggcagcatcaggaacgagacatatgaccacaacgtgtaccgggacgaggccctgaacaaccggttccagatcaagggcgtggagctgaagtccggctacaaggactggatcctgtggatcagcttcgccatgagctgcttcctgctgtgcatcgccctgctgggcttcatcatgtgggcctgccagaagggcaacatcaggtgcaacatctgcatctgatagtaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttc

SEQ ID NO: 156 - DNA, 12622 bp, synthetic construct
  source 1..12622: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggaccgacagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctcagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctcctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtcctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccacacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgacggagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtaccggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacgcccgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaaccgggtgatcaccagggaggagctggaggccctgacccctagcaggacccctagcaggagcgtgagcaggaccagcctggtgagcaaccctcccggcgtgaaccgggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtcctggagaggacggagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctccaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtcctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaaccgggtgggcatcctgagcgagctgtgcaaggccgtggagagcaggtacgagacagtgggcaccagcatcatcgtgatggccatgaccaccctggccagcagcgtcaagtccttcagctacctgaggggggcccctataactctctacggctaacctgaatggactacgacatagtctagtccgccaaggcccttaagttaaaacagctgtgggttgtcacccacccacagggtccactgggcgctagtacactggtatctcggtacctttgtacgcctgttttataccccctccctgatttgcaacttagaagcaacgcaaaccagatcaatagtaggtgtgacataccagtcgcatcttgatcaagcacttctgtatccccggaccgagtatcaatagactgtgcacacggttgaaggagaaaacgtccgttacccggctaactacttcgagaagcctagtaacgccattgaagttgcagagtgtttcgctcagcactccccccgtgtagatcaggtcgatgagtcaccgcattccccacgggcgaccgtggcggtggctgcgttggcggcctgcctatggggtaacccataggacgctctaatacggacatggcgtgaagagtctattgagctagttagtagtcctccggcccctgaatgcggctaatcctaactgcggagcacatacccttaatccaaagggcagtgtgtcgtaacgggcaactctgcagcggaaccgactactttgggtgtccgtgtttctttttattcttgtattggctgcttatggtgacaattaaagaattgttaccatatagctattggattggccatccagtgtcaaacagagctattgtatatctctttgttggattcacacctctcactcttgaaacgttacacaccctcaattacattatactgctgaacacgaagcgatgaatcctaaccagaagatcatcaccatcggcagcgtgagcctgaccatcagcaccatctgcttcttcatgcagatcgccatcctgatcaccaccgtgaccctgcacttcaagcagtacgagttcaacagccctcccaacaaccaggtgatgctgtgcgagcccaccatcatcgagaggaacatgaccgagatcgtgtacctgaccaacaccaccatcgagaaggagatctgccccaagcccgccgagtacaggaactggagcaagccccagtgcggcatcaccggcttcgcccctttcagcaaggacaacagcatcaggctgagcgccggcggcgacatctgggtgaccagggagccctacgtgagctgcgacctggacaagtgctaccagttcgccctgggccagggcaccaccctgaacaacgtgcacagcaacaacaccgtgagggacaggaccccttaccggaccctgctgatgaacgagctgggcgtgcccttccacctgggcaccaagcaggtgtgcatcgcctggagcagctccagctgccacgacggcaaggcctggctgcacgtgtgcatcaccggcgacgacaagaacgccaccgccagcttcatctacaacggcaggctggtggacagcgtggtgagctggagcaacgacatcctgaggacccaggagagcgagtgcgtgtgcatcaacggcacctgcaccgtggtgatgaccgacggcaacgccaccggcaaggccgacaccaagatcctgttcatcgaggagggcaagatcgtgcacaccagcaagctgagcggcagcgcccagcacgtggaggagtgcagctgctaccccaggtaccccggcgtgaggtgcgtgtgcagggacaactggaagggcagcaaccggcccatcatcgacatcaacatcaaggaccacagcatcgtcagcaggtacgtgtgcagcggcctggtgggcgacacccctagaaagagcgacagcagctccagcagccactgcctgaaccccaacaacgagaagggcgaccacggcgtgaagggctgggccttcgacgacggcaacgacgtgtggatgggcaggaccatcaacgagacaagcaggctgggctacgagacattcaaggtggtggagggctggagcaaccccaagagcaagctgcagatcaacaggcaggtgatcgtggacaggggcgacaggagcggctacagcggcatcttcagcgtggagggcaagagctgcatcaaccggtgcttctacgtggagctgatcaggggcaggaaggaggagacagaggtgctgtggaccagcaacagcatcgtggtgttctgcggcaccagcggcacctacggcaccggcagctggcccgacggcgccaacctgagcctgatgcacatctgatagtaaactcgaagcagtgttaaatcattcagctacctgagaggggcccctataactctctacggctaacctgaatggactacgacatagtctagtccgccaaggccgaattcttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgaagaccatcatcgccctgagctacatcctgtgcctggtgttcgcccagaagatccccggcaatgacaacagcaccgccaccctgtgcctgggccaccacgccgtgcccaacggcaccatcgtgaagaccatcaccaacgacaggatcgaggtgaccaacgccaccgagctggtgcagaacagcagcatcggcgagatctgcgacagccctcaccagatcctggacggcggcaactgcaccctgatcgacgccctgctgggcgaccctcagtgcgacggcttccagaacaaggagtgggacctgttcgtggagaggagcagggccaacagcaactgctacccctacgacgtgcccgactacgccagcctgaggagcctggtggccagcagcggcaccctggagttcaagaacgagagcttcaactggaccggcgtgaagcagaacggcaccagcagcgcctgcatcaggggcagcagctccagcttcttcagccggctgaattggctcacccacctgaactacacctaccccgccctgaacgtgaccatgcccaacaacgagcagttcgacaagctgtatatctggggcgtgcaccaccccagcaccgacaaggaccagatcagcctgttcgcccagcccagcggcaggatcaccgtgagcaccaagaggagccagcaggccgtgatccccaacatcggcagcaggcccaggatcagggacatccccagcaggatcagcatctactggaccatcgtgaagcccggcgacatcctgctgatcaacagcaccggcaacctgatcgcccctaggggctacttcaagatcaggagcggcaagagcagcatcatgaggagcgacgcccctatcggcaagtgcaagagcgagtgcatcacccctaacggcagcatccccaacgacaagcccttccagaacgtgaacaggatcacctacggcgcctgccccaggtacgtgaagcagagcaccctgaagctggccaccggcatgaggaacgtgcccgagaagcagaccaggggcatcttcggcgccatcgccggcttcatcgagaacggctgggagggcatggtggacggctggtacggcttcaggcaccagaacagcgagggcaggggccaggccgccgacctgaagtccacccaggccgccatcgaccagatcaacggcaagctgaacaggctgatcggcaagaccaacgagaagttccaccagatcgagaaggagttcagcgaggtggagggcagggtgcaggacctggagaagtacgtggaggacaccaagatcgacctgtggagctacaacgccgagctgctggtggccctggagaaccagcacaccatcgacctgaccgacagcgagatgaacaagctgttcgagaagaccaagaagcagctgagggagaacgccgaggacatgggcaacggctgcttcaagatctaccacaagtgcgacaacgcctgcatcggcagcatcaggaacgagacatatgaccacaacgtgtaccgggacgaggccctgaacaaccggttccagatcaagggcgtggagctgaagtccggctacaaggactggatcctgtggatcagcttcgccatgagctgcttcctgctgtgcatcgccctgctgggcttcatcatgtgggcctgccagaagggcaacatcaggtgcaacatctgcatctgatagtaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttc

SEQ ID NO: 157 - DNA, 12450 bp, synthetic construct
  source 1..12450: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggaccgacagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctcagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctcctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtcctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccacacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgacggagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtaccggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacgcccgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaaccgggtgatcaccagggaggagctggaggccctgacccctagcaggacccctagcaggagcgtgagcaggaccagcctggtgagcaaccctcccggcgtgaaccgggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtcctggagaggacggagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctccaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtcctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaaccgggtgggcatcctgagcgagctgtgcaaggccgtggagagcaggtacgagacagtgggcaccagcatcatcgtgatggccatgaccaccctggccagcagcgtcaagtccttcagctacctgaggggggcccctataactctctacggctaacctgaatggactacgacatagtctagtccgccaaggcccttaagttaaaacagctgtgggttgtcacccacccacagggtccactgggcgctagtacactggtatctcggtacctttgtacgcctgttttataccccctccctgatttgcaacttagaagcaacgcaaaccagatcaatagtaggtgtgacataccagtcgcatcttgatcaagcacttctgtatccccggaccgagtatcaatagactgtgcacacggttgaaggagaaaacgtccgttacccggctaactacttcgagaagcctagtaacgccattgaagttgcagagtgtttcgctcagcactccccccgtgtagatcaggtcgatgagtcaccgcattccccacgggcgaccgtggcggtggctgcgttggcggcctgcctatggggtaacccataggacgctctaatacggacatggcgtgaagagtctattgagctagttagtagtcctccggcccctgaatgcggctaatcctaactgcggagcacatacccttaatccaaagggcagtgtgtcgtaacgggcaactctgcagcggaaccgactactttgggtgtccgtgtttctttttattcttgtattggctgcttatggtgacaattaaagaattgttaccatatagctattggattggccatccagtgtcaaacagagctattgtatatctctttgttggattcacacctctcactcttgaaacgttacacaccctcaattacattatactgctgaacacgaagcgatgaatcctaaccagaagatcatcaccatcggcagcgtgagcctgaccatcagcaccatctgcttcttcatgcagatcgccatcctgatcaccaccgtgaccctgcacttcaagcagtacgagttcaacagccctcccaacaaccaggtgatgctgtgcgagcccaccatcatcgagaggaacatgaccgagatcgtgtacctgaccaacaccaccatcgagaaggagatctgccccaagcccgccgagtacaggaactggagcaagccccagtgcggcatcaccggcttcgcccctttcagcaaggacaacagcatcaggctgagcgccggcggcgacatctgggtgaccagggagccctacgtgagctgcgacctggacaagtgctaccagttcgccctgggccagggcaccaccctgaacaacgtgcacagcaacaacaccgtgagggacaggaccccttaccggaccctgctgatgaacgagctgggcgtgcccttccacctgggcaccaagcaggtgtgcatcgcctggagcagctccagctgccacgacggcaaggcctggctgcacgtgtgcatcaccggcgacgacaagaacgccaccgccagcttcatctacaacggcaggctggtggacagcgtggtgagctggagcaacgacatcctgaggacccaggagagcgagtgcgtgtgcatcaacggcacctgcaccgtggtgatgaccgacggcaacgccaccggcaaggccgacaccaagatcctgttcatcgaggagggcaagatcgtgcacaccagcaagctgagcggcagcgcccagcacgtggaggagtgcagctgctaccccaggtaccccggcgtgaggtgcgtgtgcagggacaactggaagggcagcaaccggcccatcatcgacatcaacatcaaggaccacagcatcgtcagcaggtacgtgtgcagcggcctggtgggcgacacccctagaaagagcgacagcagctccagcagccactgcctgaaccccaacaacgagaagggcgaccacggcgtgaagggctgggccttcgacgacggcaacgacgtgtggatgggcaggaccatcaacgagacaagcaggctgggctacgagacattcaaggtggtggagggctggagcaaccccaagagcaagctgcagatcaacaggcaggtgatcgtggacaggggcgacaggagcggctacagcggcatcttcagcgtggagggcaagagctgcatcaaccggtgcttctacgtggagctgatcaggggcaggaaggaggagacagaggtgctgtggaccagcaacagcatcgtggtgttctgcggcaccagcggcacctacggcaccggcagctggcccgacggcgccaacctgagcctgatgcacatctgatagtaaactcgaagcagtgttaaatcattcagctacctgagaggggcccctataactctctacggctaacctgaatggactacgacatagtctagtccgccaaggccgaattcctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataatatgaagaccatcatcgccctgagctacatcctgtgcctggtgttcgcccagaagatccccggcaatgacaacagcaccgccaccctgtgcctgggccaccacgccgtgcccaacggcaccatcgtgaagaccatcaccaacgacaggatcgaggtgaccaacgccaccgagctggtgcagaacagcagcatcggcgagatctgcgacagccctcaccagatcctggacggcggcaactgcaccctgatcgacgccctgctgggcgaccctcagtgcgacggcttccagaacaaggagtgggacctgttcgtggagaggagcagggccaacagcaactgctacccctacgacgtgcccgactacgccagcctgaggagcctggtggccagcagcggcaccctggagttcaagaacgagagcttcaactggaccggcgtgaagcagaacggcaccagcagcgcctgcatcaggggcagcagctccagcttcttcagccggctgaattggctcacccacctgaactacacctaccccgccctgaacgtgaccatgcccaacaacgagcagttcgacaagctgtatatctggggcgtgcaccaccccagcaccgacaaggaccagatcagcctgttcgcccagcccagcggcaggatcaccgtgagcaccaagaggagccagcaggccgtgatccccaacatcggcagcaggcccaggatcagggacatccccagcaggatcagcatctactggaccatcgtgaagcccggcgacatcctgctgatcaacagcaccggcaacctgatcgcccctaggggctacttcaagatcaggagcggcaagagcagcatcatgaggagcgacgcccctatcggcaagtgcaagagcgagtgcatcacccctaacggcagcatccccaacgacaagcccttccagaacgtgaacaggatcacctacggcgcctgccccaggtacgtgaagcagagcaccctgaagctggccaccggcatgaggaacgtgcccgagaagcagaccaggggcatcttcggcgccatcgccggcttcatcgagaacggctgggagggcatggtggacggctggtacggcttcaggcaccagaacagcgagggcaggggccaggccgccgacctgaagtccacccaggccgccatcgaccagatcaacggcaagctgaacaggctgatcggcaagaccaacgagaagttccaccagatcgagaaggagttcagcgaggtggagggcagggtgcaggacctggagaagtacgtggaggacaccaagatcgacctgtggagctacaacgccgagctgctggtggccctggagaaccagcacaccatcgacctgaccgacagcgagatgaacaagctgttcgagaagaccaagaagcagctgagggagaacgccgaggacatgggcaacggctgcttcaagatctaccacaagtgcgacaacgcctgcatcggcagcatcaggaacgagacatatgaccacaacgtgtaccgggacgaggccctgaacaaccggttccagatcaagggcgtggagctgaagtccggctacaaggactggatcctgtggatcagcttcgccatgagctgcttcctgctgtgcatcgccctgctgggcttcatcatgtgggcctgccagaagggcaacatcaggtgcaacatctgcatctgatagtaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttc

SEQ ID NO: 158 - DNA, 12624 bp, synthetic construct
  source 1..12624: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggaccgacagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctcagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctcctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtcctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccacacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgacggagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtaccggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacgcccgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaaccgggtgatcaccagggaggagctggaggccctgacccctagcaggacccctagcaggagcgtgagcaggaccagcctggtgagcaaccctcccggcgtgaaccgggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtcctggagaggacggagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctccaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtcctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaaccgggtgggcatcctgagcgagctgtgcaaggccgtggagagcaggtacgagacagtgggcaccagcatcatcgtgatggccatgaccaccctggccagcagcgtcaagtccttcagctacctgaggggggcccctataactctctacggctaacctgaatggactacgacatagtctagtccgccaaggcccttaagttaaaacagctgtgggttgtcacccacccacagggtccactgggcgctagtacactggtatctcggtacctttgtacgcctgttttataccccctccctgatttgcaacttagaagcaacgcaaaccagatcaatagtaggtgtgacataccagtcgcatcttgatcaagcacttctgtatccccggaccgagtatcaatagactgtgcacacggttgaaggagaaaacgtccgttacccggctaactacttcgagaagcctagtaacgccattgaagttgcagagtgtttcgctcagcactccccccgtgtagatcaggtcgatgagtcaccgcattccccacgggcgaccgtggcggtggctgcgttggcggcctgcctatggggtaacccataggacgctctaatacggacatggcgtgaagagtctattgagctagttagtagtcctccggcccctgaatgcggctaatcctaactgcggagcacatacccttaatccaaagggcagtgtgtcgtaacgggcaactctgcagcggaaccgactactttgggtgtccgtgtttctttttattcttgtattggctgcttatggtgacaattaaagaattgttaccatatagctattggattggccatccagtgtcaaacagagctattgtatatctctttgttggattcacacctctcactcttgaaacgttacacaccctcaattacattatactgctgaacacgaagcgatgaatcctaaccagaagatcatcaccatcggcagcgtgagcctgaccatcagcaccatctgcttcttcatgcagatcgccatcctgatcaccaccgtgaccctgcacttcaagcagtacgagttcaacagccctcccaacaaccaggtgatgctgtgcgagcccaccatcatcgagaggaacatgaccgagatcgtgtacctgaccaacaccaccatcgagaaggagatctgccccaagcccgccgagtacaggaactggagcaagccccagtgcggcatcaccggcttcgcccctttcagcaaggacaacagcatcaggctgagcgccggcggcgacatctgggtgaccagggagccctacgtgagctgcgacctggacaagtgctaccagttcgccctgggccagggcaccaccctgaacaacgtgcacagcaacaacaccgtgagggacaggaccccttaccggaccctgctgatgaacgagctgggcgtgcccttccacctgggcaccaagcaggtgtgcatcgcctggagcagctccagctgccacgacggcaaggcctggctgcacgtgtgcatcaccggcgacgacaagaacgccaccgccagcttcatctacaacggcaggctggtggacagcgtggtgagctggagcaacgacatcctgaggacccaggagagcgagtgcgtgtgcatcaacggcacctgcaccgtggtgatgaccgacggcaacgccaccggcaaggccgacaccaagatcctgttcatcgaggagggcaagatcgtgcacaccagcaagctgagcggcagcgcccagcacgtggaggagtgcagctgctaccccaggtaccccggcgtgaggtgcgtgtgcagggacaactggaagggcagcaaccggcccatcatcgacatcaacatcaaggaccacagcatcgtcagcaggtacgtgtgcagcggcctggtgggcgacacccctagaaagagcgacagcagctccagcagccactgcctgaaccccaacaacgagaagggcgaccacggcgtgaagggctgggccttcgacgacggcaacgacgtgtggatgggcaggaccatcaacgagacaagcaggctgggctacgagacattcaaggtggtggagggctggagcaaccccaagagcaagctgcagatcaacaggcaggtgatcgtggacaggggcgacaggagcggctacagcggcatcttcagcgtggagggcaagagctgcatcaaccggtgcttctacgtggagctgatcaggggcaggaaggaggagacagaggtgctgtggaccagcaacagcatcgtggtgttctgcggcaccagcggcacctacggcaccggcagctggcccgacggcgccaacctgagcctgatgcacatctgatagtaaactcgaagcagtgttaaatcattcagctacctgagaggggcccctataactctctacggctaacctgaatggactacgacatagtctagtccgccaaggccgaattcttaaaacagctgtgggttgtcacccacccacagggtccactgggcgctagtacactggtatctcggtacctttgtacgcctgttttataccccctccctgatttgcaacttagaagcaacgcaaaccagatcaatagtaggtgtgacataccagtcgcatcttgatcaagcacttctgtatccccggaccgagtatcaatagactgtgcacacggttgaaggagaaaacgtccgttacccggctaactacttcgagaagcctagtaacgccattgaagttgcagagtgtttcgctcagcactccccccgtgtagatcaggtcgatgagtcaccgcattccccacgggcgaccgtggcggtggctgcgttggcggcctgcctatggggtaacccataggacgctctaatacggacatggcgtgaagagtctattgagctagttagtagtcctccggcccctgaatgcggctaatcctaactgcggagcacatacccttaatccaaagggcagtgtgtcgtaacgggcaactctgcagcggaaccgactactttgggtgtccgtgtttctttttattcttgtattggctgcttatggtgacaattaaagaattgttaccatatagctattggattggccatccagtgtcaaacagagctattgtatatctctttgttggattcacacctctcactcttgaaacgttacacaccctcaattacattatactgctgaacacgaagcgatgaagaccatcatcgccctgagctacatcctgtgcctggtgttcgcccagaagatccccggcaatgacaacagcaccgccaccctgtgcctgggccaccacgccgtgcccaacggcaccatcgtgaagaccatcaccaacgacaggatcgaggtgaccaacgccaccgagctggtgcagaacagcagcatcggcgagatctgcgacagccctcaccagatcctggacggcggcaactgcaccctgatcgacgccctgctgggcgaccctcagtgcgacggcttccagaacaaggagtgggacctgttcgtggagaggagcagggccaacagcaactgctacccctacgacgtgcccgactacgccagcctgaggagcctggtggccagcagcggcaccctggagttcaagaacgagagcttcaactggaccggcgtgaagcagaacggcaccagcagcgcctgcatcaggggcagcagctccagcttcttcagccggctgaattggctcacccacctgaactacacctaccccgccctgaacgtgaccatgcccaacaacgagcagttcgacaagctgtatatctggggcgtgcaccaccccagcaccgacaaggaccagatcagcctgttcgcccagcccagcggcaggatcaccgtgagcaccaagaggagccagcaggccgtgatccccaacatcggcagcaggcccaggatcagggacatccccagcaggatcagcatctactggaccatcgtgaagcccggcgacatcctgctgatcaacagcaccggcaacctgatcgcccctaggggctacttcaagatcaggagcggcaagagcagcatcatgaggagcgacgcccctatcggcaagtgcaagagcgagtgcatcacccctaacggcagcatccccaacgacaagcccttccagaacgtgaacaggatcacctacggcgcctgccccaggtacgtgaagcagagcaccctgaagctggccaccggcatgaggaacgtgcccgagaagcagaccaggggcatcttcggcgccatcgccggcttcatcgagaacggctgggagggcatggtggacggctggtacggcttcaggcaccagaacagcgagggcaggggccaggccgccgacctgaagtccacccaggccgccatcgaccagatcaacggcaagctgaacaggctgatcggcaagaccaacgagaagttccaccagatcgagaaggagttcagcgaggtggagggcagggtgcaggacctggagaagtacgtggaggacaccaagatcgacctgtggagctacaacgccgagctgctggtggccctggagaaccagcacaccatcgacctgaccgacagcgagatgaacaagctgttcgagaagaccaagaagcagctgagggagaacgccgaggacatgggcaacggctgcttcaagatctaccacaagtgcgacaacgcctgcatcggcagcatcaggaacgagacatatgaccacaacgtgtaccgggacgaggccctgaacaaccggttccagatcaagggcgtggagctgaagtccggctacaaggactggatcctgtggatcagcttcgccatgagctgcttcctgctgtgcatcgccctgctgggcttcatcatgtgggcctgccagaagggcaacatcaggtgcaacatctgcatctgatagtaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttc

SEQ ID NO: 159 - DNA, 12620 bp, synthetic construct
  source 1..12620: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggaccgacagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctcagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctcctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtcctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccacacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgacggagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtaccggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacgcccgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaaccgggtgatcaccagggaggagctggaggccctgacccctagcaggacccctagcaggagcgtgagcaggaccagcctggtgagcaaccctcccggcgtgaaccgggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtcctggagaggacggagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctccaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtcctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaaccgggtgggcatcctgagcgagctgtgcaaggccgtggagagcaggtacgagacagtgggcaccagcatcatcgtgatggccatgaccaccctggccagcagcgtcaagtccttcagctacctgaggggggcccctataactctctacggctaacctgaatggactacgacatagtctagtccgccaaggcccttaagttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgaatcctaaccagaagatcatcaccatcggcagcgtgagcctgaccatcagcaccatctgcttcttcatgcagatcgccatcctgatcaccaccgtgaccctgcacttcaagcagtacgagttcaacagccctcccaacaaccaggtgatgctgtgcgagcccaccatcatcgagaggaacatgaccgagatcgtgtacctgaccaacaccaccatcgagaaggagatctgccccaagcccgccgagtacaggaactggagcaagccccagtgcggcatcaccggcttcgcccctttcagcaaggacaacagcatcaggctgagcgccggcggcgacatctgggtgaccagggagccctacgtgagctgcgacctggacaagtgctaccagttcgccctgggccagggcaccaccctgaacaacgtgcacagcaacaacaccgtgagggacaggaccccttaccggaccctgctgatgaacgagctgggcgtgcccttccacctgggcaccaagcaggtgtgcatcgcctggagcagctccagctgccacgacggcaaggcctggctgcacgtgtgcatcaccggcgacgacaagaacgccaccgccagcttcatctacaacggcaggctggtggacagcgtggtgagctggagcaacgacatcctgaggacccaggagagcgagtgcgtgtgcatcaacggcacctgcaccgtggtgatgaccgacggcaacgccaccggcaaggccgacaccaagatcctgttcatcgaggagggcaagatcgtgcacaccagcaagctgagcggcagcgcccagcacgtggaggagtgcagctgctaccccaggtaccccggcgtgaggtgcgtgtgcagggacaactggaagggcagcaaccggcccatcatcgacatcaacatcaaggaccacagcatcgtcagcaggtacgtgtgcagcggcctggtgggcgacacccctagaaagagcgacagcagctccagcagccactgcctgaaccccaacaacgagaagggcgaccacggcgtgaagggctgggccttcgacgacggcaacgacgtgtggatgggcaggaccatcaacgagacaagcaggctgggctacgagacattcaaggtggtggagggctggagcaaccccaagagcaagctgcagatcaacaggcaggtgatcgtggacaggggcgacaggagcggctacagcggcatcttcagcgtggagggcaagagctgcatcaaccggtgcttctacgtggagctgatcaggggcaggaaggaggagacagaggtgctgtggaccagcaacagcatcgtggtgttctgcggcaccagcggcacctacggcaccggcagctggcccgacggcgccaacctgagcctgatgcacatctgatagtaaactcgaagcagtgttaaatcattcagctacctgagaggggcccctataactctctacggctaacctgaatggactacgacatagtctagtccgccaaggccgaattcttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgaagaccatcatcgccctgagctacatcctgtgcctggtgttcgcccagaagatccccggcaatgacaacagcaccgccaccctgtgcctgggccaccacgccgtgcccaacggcaccatcgtgaagaccatcaccaacgacaggatcgaggtgaccaacgccaccgagctggtgcagaacagcagcatcggcgagatctgcgacagccctcaccagatcctggacggcggcaactgcaccctgatcgacgccctgctgggcgaccctcagtgcgacggcttccagaacaaggagtgggacctgttcgtggagaggagcagggccaacagcaactgctacccctacgacgtgcccgactacgccagcctgaggagcctggtggccagcagcggcaccctggagttcaagaacgagagcttcaactggaccggcgtgaagcagaacggcaccagcagcgcctgcatcaggggcagcagctccagcttcttcagccggctgaattggctcacccacctgaactacacctaccccgccctgaacgtgaccatgcccaacaacgagcagttcgacaagctgtatatctggggcgtgcaccaccccagcaccgacaaggaccagatcagcctgttcgcccagcccagcggcaggatcaccgtgagcaccaagaggagccagcaggccgtgatccccaacatcggcagcaggcccaggatcagggacatccccagcaggatcagcatctactggaccatcgtgaagcccggcgacatcctgctgatcaacagcaccggcaacctgatcgcccctaggggctacttcaagatcaggagcggcaagagcagcatcatgaggagcgacgcccctatcggcaagtgcaagagcgagtgcatcacccctaacggcagcatccccaacgacaagcccttccagaacgtgaacaggatcacctacggcgcctgccccaggtacgtgaagcagagcaccctgaagctggccaccggcatgaggaacgtgcccgagaagcagaccaggggcatcttcggcgccatcgccggcttcatcgagaacggctgggagggcatggtggacggctggtacggcttcaggcaccagaacagcgagggcaggggccaggccgccgacctgaagtccacccaggccgccatcgaccagatcaacggcaagctgaacaggctgatcggcaagaccaacgagaagttccaccagatcgagaaggagttcagcgaggtggagggcagggtgcaggacctggagaagtacgtggaggacaccaagatcgacctgtggagctacaacgccgagctgctggtggccctggagaaccagcacaccatcgacctgaccgacagcgagatgaacaagctgttcgagaagaccaagaagcagctgagggagaacgccgaggacatgggcaacggctgcttcaagatctaccacaagtgcgacaacgcctgcatcggcagcatcaggaacgagacatatgaccacaacgtgtaccgggacgaggccctgaacaaccggttccagatcaagggcgtggagctgaagtccggctacaaggactggatcctgtggatcagcttcgccatgagctgcttcctgctgtgcatcgccctgctgggcttcatcatgtgggcctgccagaagggcaacatcaggtgcaacatctgcatctgatagtaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttc

SEQ ID NO: 160 - DNA, 12622 bp, synthetic construct
  source 1..12622: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggaccgacagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctcagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctcctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtcctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccacacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgacggagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtaccggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacgcccgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaaccgggtgatcaccagggaggagctggaggccctgacccctagcaggacccctagcaggagcgtgagcaggaccagcctggtgagcaaccctcccggcgtgaaccgggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtcctggagaggacggagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctccaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtcctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaaccgggtgggcatcctgagcgagctgtgcaaggccgtggagagcaggtacgagacagtgggcaccagcatcatcgtgatggccatgaccaccctggccagcagcgtcaagtccttcagctacctgaggggggcccctataactctctacggctaacctgaatggactacgacatagtctagtccgccaaggcccttaagttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgaatcctaaccagaagatcatcaccatcggcagcgtgagcctgaccatcagcaccatctgcttcttcatgcagatcgccatcctgatcaccaccgtgaccctgcacttcaagcagtacgagttcaacagccctcccaacaaccaggtgatgctgtgcgagcccaccatcatcgagaggaacatgaccgagatcgtgtacctgaccaacaccaccatcgagaaggagatctgccccaagcccgccgagtacaggaactggagcaagccccagtgcggcatcaccggcttcgcccctttcagcaaggacaacagcatcaggctgagcgccggcggcgacatctgggtgaccagggagccctacgtgagctgcgacctggacaagtgctaccagttcgccctgggccagggcaccaccctgaacaacgtgcacagcaacaacaccgtgagggacaggaccccttaccggaccctgctgatgaacgagctgggcgtgcccttccacctgggcaccaagcaggtgtgcatcgcctggagcagctccagctgccacgacggcaaggcctggctgcacgtgtgcatcaccggcgacgacaagaacgccaccgccagcttcatctacaacggcaggctggtggacagcgtggtgagctggagcaacgacatcctgaggacccaggagagcgagtgcgtgtgcatcaacggcacctgcaccgtggtgatgaccgacggcaacgccaccggcaaggccgacaccaagatcctgttcatcgaggagggcaagatcgtgcacaccagcaagctgagcggcagcgcccagcacgtggaggagtgcagctgctaccccaggtaccccggcgtgaggtgcgtgtgcagggacaactggaagggcagcaaccggcccatcatcgacatcaacatcaaggaccacagcatcgtcagcaggtacgtgtgcagcggcctggtgggcgacacccctagaaagagcgacagcagctccagcagccactgcctgaaccccaacaacgagaagggcgaccacggcgtgaagggctgggccttcgacgacggcaacgacgtgtggatgggcaggaccatcaacgagacaagcaggctgggctacgagacattcaaggtggtggagggctggagcaaccccaagagcaagctgcagatcaacaggcaggtgatcgtggacaggggcgacaggagcggctacagcggcatcttcagcgtggagggcaagagctgcatcaaccggtgcttctacgtggagctgatcaggggcaggaaggaggagacagaggtgctgtggaccagcaacagcatcgtggtgttctgcggcaccagcggcacctacggcaccggcagctggcccgacggcgccaacctgagcctgatgcacatctgatagtaaactcgaagcagtgttaaatcattcagctacctgagaggggcccctataactctctacggctaacctgaatggactacgacatagtctagtccgccaaggccgaattcttaaaacagctgtgggttgtcacccacccacagggtccactgggcgctagtacactggtatctcggtacctttgtacgcctgttttataccccctccctgatttgcaacttagaagcaacgcaaaccagatcaatagtaggtgtgacataccagtcgcatcttgatcaagcacttctgtatccccggaccgagtatcaatagactgtgcacacggttgaaggagaaaacgtccgttacccggctaactacttcgagaagcctagtaacgccattgaagttgcagagtgtttcgctcagcactccccccgtgtagatcaggtcgatgagtcaccgcattccccacgggcgaccgtggcggtggctgcgttggcggcctgcctatggggtaacccataggacgctctaatacggacatggcgtgaagagtctattgagctagttagtagtcctccggcccctgaatgcggctaatcctaactgcggagcacatacccttaatccaaagggcagtgtgtcgtaacgggcaactctgcagcggaaccgactactttgggtgtccgtgtttctttttattcttgtattggctgcttatggtgacaattaaagaattgttaccatatagctattggattggccatccagtgtcaaacagagctattgtatatctctttgttggattcacacctctcactcttgaaacgttacacaccctcaattacattatactgctgaacacgaagcgatgaagaccatcatcgccctgagctacatcctgtgcctggtgttcgcccagaagatccccggcaatgacaacagcaccgccaccctgtgcctgggccaccacgccgtgcccaacggcaccatcgtgaagaccatcaccaacgacaggatcgaggtgaccaacgccaccgagctggtgcagaacagcagcatcggcgagatctgcgacagccctcaccagatcctggacggcggcaactgcaccctgatcgacgccctgctgggcgaccctcagtgcgacggcttccagaacaaggagtgggacctgttcgtggagaggagcagggccaacagcaactgctacccctacgacgtgcccgactacgccagcctgaggagcctggtggccagcagcggcaccctggagttcaagaacgagagcttcaactggaccggcgtgaagcagaacggcaccagcagcgcctgcatcaggggcagcagctccagcttcttcagccggctgaattggctcacccacctgaactacacctaccccgccctgaacgtgaccatgcccaacaacgagcagttcgacaagctgtatatctggggcgtgcaccaccccagcaccgacaaggaccagatcagcctgttcgcccagcccagcggcaggatcaccgtgagcaccaagaggagccagcaggccgtgatccccaacatcggcagcaggcccaggatcagggacatccccagcaggatcagcatctactggaccatcgtgaagcccggcgacatcctgctgatcaacagcaccggcaacctgatcgcccctaggggctacttcaagatcaggagcggcaagagcagcatcatgaggagcgacgcccctatcggcaagtgcaagagcgagtgcatcacccctaacggcagcatccccaacgacaagcccttccagaacgtgaacaggatcacctacggcgcctgccccaggtacgtgaagcagagcaccctgaagctggccaccggcatgaggaacgtgcccgagaagcagaccaggggcatcttcggcgccatcgccggcttcatcgagaacggctgggagggcatggtggacggctggtacggcttcaggcaccagaacagcgagggcaggggccaggccgccgacctgaagtccacccaggccgccatcgaccagatcaacggcaagctgaacaggctgatcggcaagaccaacgagaagttccaccagatcgagaaggagttcagcgaggtggagggcagggtgcaggacctggagaagtacgtggaggacaccaagatcgacctgtggagctacaacgccgagctgctggtggccctggagaaccagcacaccatcgacctgaccgacagcgagatgaacaagctgttcgagaagaccaagaagcagctgagggagaacgccgaggacatgggcaacggctgcttcaagatctaccacaagtgcgacaacgcctgcatcggcagcatcaggaacgagacatatgaccacaacgtgtaccgggacgaggccctgaacaaccggttccagatcaagggcgtggagctgaagtccggctacaaggactggatcctgtggatcagcttcgccatgagctgcttcctgctgtgcatcgccctgctgggcttcatcatgtgggcctgccagaagggcaacatcaggtgcaacatctgcatctgatagtaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttc

SEQ ID NO: 161 - DNA, 12448 bp, synthetic construct
  source 1..12448: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggaccgacagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctcagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctcctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtcctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccacacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgacggagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtaccggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacgcccgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaaccgggtgatcaccagggaggagctggaggccctgacccctagcaggacccctagcaggagcgtgagcaggaccagcctggtgagcaaccctcccggcgtgaaccgggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtcctggagaggacggagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctccaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtcctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaaccgggtgggcatcctgagcgagctgtgcaaggccgtggagagcaggtacgagacagtgggcaccagcatcatcgtgatggccatgaccaccctggccagcagcgtcaagtccttcagctacctgaggggggcccctataactctctacggctaacctgaatggactacgacatagtctagtccgccaaggcccttaagctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataatatgaatcctaaccagaagatcatcaccatcggcagcgtgagcctgaccatcagcaccatctgcttcttcatgcagatcgccatcctgatcaccaccgtgaccctgcacttcaagcagtacgagttcaacagccctcccaacaaccaggtgatgctgtgcgagcccaccatcatcgagaggaacatgaccgagatcgtgtacctgaccaacaccaccatcgagaaggagatctgccccaagcccgccgagtacaggaactggagcaagccccagtgcggcatcaccggcttcgcccctttcagcaaggacaacagcatcaggctgagcgccggcggcgacatctgggtgaccagggagccctacgtgagctgcgacctggacaagtgctaccagttcgccctgggccagggcaccaccctgaacaacgtgcacagcaacaacaccgtgagggacaggaccccttaccggaccctgctgatgaacgagctgggcgtgcccttccacctgggcaccaagcaggtgtgcatcgcctggagcagctccagctgccacgacggcaaggcctggctgcacgtgtgcatcaccggcgacgacaagaacgccaccgccagcttcatctacaacggcaggctggtggacagcgtggtgagctggagcaacgacatcctgaggacccaggagagcgagtgcgtgtgcatcaacggcacctgcaccgtggtgatgaccgacggcaacgccaccggcaaggccgacaccaagatcctgttcatcgaggagggcaagatcgtgcacaccagcaagctgagcggcagcgcccagcacgtggaggagtgcagctgctaccccaggtaccccggcgtgaggtgcgtgtgcagggacaactggaagggcagcaaccggcccatcatcgacatcaacatcaaggaccacagcatcgtcagcaggtacgtgtgcagcggcctggtgggcgacacccctagaaagagcgacagcagctccagcagccactgcctgaaccccaacaacgagaagggcgaccacggcgtgaagggctgggccttcgacgacggcaacgacgtgtggatgggcaggaccatcaacgagacaagcaggctgggctacgagacattcaaggtggtggagggctggagcaaccccaagagcaagctgcagatcaacaggcaggtgatcgtggacaggggcgacaggagcggctacagcggcatcttcagcgtggagggcaagagctgcatcaaccggtgcttctacgtggagctgatcaggggcaggaaggaggagacagaggtgctgtggaccagcaacagcatcgtggtgttctgcggcaccagcggcacctacggcaccggcagctggcccgacggcgccaacctgagcctgatgcacatctgatagtaaactcgaagcagtgttaaatcattcagctacctgagaggggcccctataactctctacggctaacctgaatggactacgacatagtctagtccgccaaggccgaattcttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgaagaccatcatcgccctgagctacatcctgtgcctggtgttcgcccagaagatccccggcaatgacaacagcaccgccaccctgtgcctgggccaccacgccgtgcccaacggcaccatcgtgaagaccatcaccaacgacaggatcgaggtgaccaacgccaccgagctggtgcagaacagcagcatcggcgagatctgcgacagccctcaccagatcctggacggcggcaactgcaccctgatcgacgccctgctgggcgaccctcagtgcgacggcttccagaacaaggagtgggacctgttcgtggagaggagcagggccaacagcaactgctacccctacgacgtgcccgactacgccagcctgaggagcctggtggccagcagcggcaccctggagttcaagaacgagagcttcaactggaccggcgtgaagcagaacggcaccagcagcgcctgcatcaggggcagcagctccagcttcttcagccggctgaattggctcacccacctgaactacacctaccccgccctgaacgtgaccatgcccaacaacgagcagttcgacaagctgtatatctggggcgtgcaccaccccagcaccgacaaggaccagatcagcctgttcgcccagcccagcggcaggatcaccgtgagcaccaagaggagccagcaggccgtgatccccaacatcggcagcaggcccaggatcagggacatccccagcaggatcagcatctactggaccatcgtgaagcccggcgacatcctgctgatcaacagcaccggcaacctgatcgcccctaggggctacttcaagatcaggagcggcaagagcagcatcatgaggagcgacgcccctatcggcaagtgcaagagcgagtgcatcacccctaacggcagcatccccaacgacaagcccttccagaacgtgaacaggatcacctacggcgcctgccccaggtacgtgaagcagagcaccctgaagctggccaccggcatgaggaacgtgcccgagaagcagaccaggggcatcttcggcgccatcgccggcttcatcgagaacggctgggagggcatggtggacggctggtacggcttcaggcaccagaacagcgagggcaggggccaggccgccgacctgaagtccacccaggccgccatcgaccagatcaacggcaagctgaacaggctgatcggcaagaccaacgagaagttccaccagatcgagaaggagttcagcgaggtggagggcagggtgcaggacctggagaagtacgtggaggacaccaagatcgacctgtggagctacaacgccgagctgctggtggccctggagaaccagcacaccatcgacctgaccgacagcgagatgaacaagctgttcgagaagaccaagaagcagctgagggagaacgccgaggacatgggcaacggctgcttcaagatctaccacaagtgcgacaacgcctgcatcggcagcatcaggaacgagacatatgaccacaacgtgtaccgggacgaggccctgaacaaccggttccagatcaagggcgtggagctgaagtccggctacaaggactggatcctgtggatcagcttcgccatgagctgcttcctgctgtgcatcgccctgctgggcttcatcatgtgggcctgccagaagggcaacatcaggtgcaacatctgcatctgatagtaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttc

SEQ ID NO: 162 - DNA, 815 bp, synthetic construct
  source 1..815: mol_type = other DNA
actcgaccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 163 - DNA, 741 bp, synthetic construct
  source 1..741: mol_type = other DNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 164 - DNA, 567 bp, synthetic construct
  source 1..567: mol_type = other DNA
tccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataa

SEQ ID NO: 165 - DNA, 743 bp, synthetic construct
  source 1..743: mol_type = other DNA
ttaaaacagctgtgggttgtcacccacccacagggtccactgggcgctagtacactggtatctcggtacctttgtacgcctgttttataccccctccctgatttgcaacttagaagcaacgcaaaccagatcaatagtaggtgtgacataccagtcgcatcttgatcaagcacttctgtatccccggaccgagtatcaatagactgtgcacacggttgaaggagaaaacgtccgttacccggctaactacttcgagaagcctagtaacgccattgaagttgcagagtgtttcgctcagcactccccccgtgtagatcaggtcgatgagtcaccgcattccccacgggcgaccgtggcggtggctgcgttggcggcctgcctatggggtaacccataggacgctctaatacggacatggcgtgaagagtctattgagctagttagtagtcctccggcccctgaatgcggctaatcctaactgcggagcacatacccttaatccaaagggcagtgtgtcgtaacgggcaactctgcagcggaaccgactactttgggtgtccgtgtttctttttattcttgtattggctgcttatggtgacaattaaagaattgttaccatatagctattggattggccatccagtgtcaaacagagctattgtatatctctttgttggattcacacctctcactcttgaaacgttacacaccctcaattacattatactgctgaacacgaagcg

(19) United States
(12) Patent Application Publication
(10) Pub. No.: US 2024/0301007 A1
(43) Pub. Date: Sep. 12, 2024

(54) METHODS AND COMPOSITIONS FOR QUADRIVALENT INFLUENZA VACCINE

(71) Applicant: Arcturus Therapeutics, Inc., San Diego, CA (US)

(72) Inventors: Brian SULLIVAN, San Diego, CA (US); Sean SULLIVAN, San Diego, CA (US); Daiki MATSUDA, San Diego, CA (US); Rodrigo YELIN, San Diego, CA (US); Jinho PARK, San Diego, CA (US)

(21) Appl. No.: 18/427,625

(22) Filed: Jan. 30, 2024

Related U.S. Application Data
(60) Provisional application No. 63/482,560, filed on Jan. 31, 2023.

(51) Int. Cl. C07K 14/005; A61K 9/127; A61P 31/16

(52) U.S. Cl. CPC C07K 14/005; A61K 9/1272; A61P 31/16; C12N 2760/16121; C12N 2760/16134; C12N 2770/36122; C12N 2770/36134

(57) ABSTRACT

Provided herein are RNA molecules encoding viral replication proteins and antigenic proteins or fragments thereof. Also provided herein are compositions that include RNA molecules encoding viral replication proteins and antigenic proteins or fragments thereof, and lipids. RNA molecules and compositions including them are useful for inducing immune responses.

### CROSS-REFERENCE TO RELATED APPLICATION

This application claims priority to U.S. Provisional Application Ser. No. 63/482,560, filed on Jan. 31, 2023, the disclosure of which is hereby incorporated by reference in its entirety.

### INCORPORATION OF SEQUENCE LISTING

The Sequence Listing titled 207653-603601_SL.xml, which was created on Jan. 8, 2024 and is 367,117 bytes in size, is hereby incorporated by reference in its entirety

### TECHNICAL FIELD

The present disclosure relates generally to inducing immune responses against infectious agents, such as influenza viruses, and more specifically to RNA molecules and liponanoparticles as vaccines.

### BACKGROUND

Influenza viruses cause epidemics almost every winter. Influenza infection results in various disease states, from a sub-clinical infections to severe viral pneumonia. The severity of the disease is affected by the age of the host, their immune status, and/or the site of infection. Vaccination plays a critical role in controlling seasonal influenza epidemics.

Self-replicating ribonucleic acids (RNAs), e.g., RNAs derived from viral replicons, and messenger RNAs (mRNAs) are useful for expression of proteins, such as heterologous proteins, for a variety of purposes, such as expression of therapeutic proteins and expression of antigens for vaccines. A desirable property of replicons is the ability for sustained expression of the protein. Few treatments for infections caused by viruses and eukaryotic organisms are available, and resistance to antibiotics for the treatment of bacterial infections is increasing. In addition, rapid responses, including rapid vaccine development, are required to effectively control emerging infectious diseases and pandemics. Thus, there exists a need for the prevention and/or treatment of infectious diseases, such as influenza.

### SUMMARY

The present disclosure provides RNA molecules that are useful for inducing immune responses. Both self-replicating RNA molecules and messenger RNA (mRNA) molecules are provided, as well as polynucleotides encoding the same.

In some aspects, provided herein are compositions comprising one or more RNA molecules, wherein the one or more RNA molecules collectively encode a hemagglutinin (HA) polypeptide and a neuraminidase (NA) polypeptide of each of four different strains of influenza virus. In some embodiments, the one or more RNA molecules comprise 8 RNA molecules (e.g., one for each NA and one for each HA). In some embodiments, two or more influenza polypeptides (e.g., two HA, two NA, or an HA and an NA) are encoded on a single RNA molecules, such that fewer than 8 RNA molecules encode the HA and NA polypeptides of the four different strains (e.g., 7, 6, 5, 4, 3, or 2 RNA molecules). In some embodiments, the HA polypeptide and the NA polypeptide are encoded by the same RNA molecule for each of the four strains of influenza virus. In some embodiments, the HA and NA polypeptides of the four different strains are all encoded on the same RNA molecule.

In some embodiments, the HA and NA polypeptides of a first strain of influenza virus are encoded by a first RNA molecule, the HA and NA polypeptides of a second strain of influenza virus are encoded by a second RNA molecule, the HA and NA polypeptides of a third strain of influenza virus are encoded by a third RNA molecule, and the HA and NA polypeptides of a fourth strain of influenza virus are encoded by a fourth RNA molecule. In some embodiments, the first, second, third, and fourth RNA molecules are present in an equimolar ratio.

In some embodiments, each of the one or more RNA molecules further encodes one or more viral replication proteins. In yet further embodiments, the one or more viral replication proteins are alphavirus proteins. In some embodiments, each of the one or more RNA molecules encodes in 5′ to 3′ order: (i) the one or more viral replication proteins, (ii) one of the NA polypeptides; and (iii) one of the HA polypeptides. In yet some embodiments, the alphavirus proteins are from Venezuelan Equine Encephalitis Virus (VEEV). In some embodiments, the one or more viral replication proteins comprise an alphavirus nonstructural protein 1 (nsP1), an alphavirus nonstructural protein 2 (nsP2), an alphavirus nonstructural protein 3 (nsP3), an alphavirus nonstructural protein 4 (nsP4), or any combination thereof. In some embodiments, the one or more viral replication proteins comprises a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, or 100% identity to the RNA sequence encoded by SEQ ID NO: 13.

In some embodiments, the sequences encoding the HA and NA polypeptides of at least one of the one or more RNA molecules are preceded by a subgenomic promoter (sgP). In some embodiments, each HA polypeptide comprises an antigenic fragment of a respective HA protein. In yet further embodiments, each NA polypeptide comprises an antigenic fragment of a respective NA protein. In some embodiments, the four different strains of influenza virus comprise one or more of H1N1, H3N2, Victoria-B, or Yamagata-B. In further embodiments, the four different strains of influenza virus comprise Victoria B/Austria/1359417/2021, H3N2 A/Darwin/6/2021, H1N1 A/Wisconsin/588/2019, and Yamagata B/PHUKET/3073/2013.

In some embodiments, each of the one or more RNA molecules further comprise a 5′ untranslated region (UTR). In some embodiments, at least one 5′ UTR comprises a viral 5′ UTR, a non-viral 5′ UTR, or a combination of viral and non-viral 5′ UTR sequences. In further embodiments, the at least one 5′ UTR comprises an alphavirus 5′ UTR. In yet further embodiments, the alphavirus 5′ UTR comprises a Venezuelan Equine Encephalitis Virus (VEEV) 5′ UTR sequence. In some embodiments, at least one 5′ UTR comprises the RNA sequence encoded by SEQ ID NO: 14. In some embodiments, each of the one or more RNA molecules further comprise a 3′ untranslated region (UTR). In further embodiments, at least one 3′ UTR comprises a viral 3′ UTR, a non-viral 3′ UTR, or a combination of viral and non-viral 3′ UTR sequences. In yet further embodiments, the at least one 3′ UTR comprises an alphavirus 3′ UTR sequence. In some embodiments, the alphavirus 3′ UTR comprises a Venezuelan Equine Encephalitis Virus (VEEV) 3′ UTR sequence. In some embodiments, at least one 3′ UTR comprises the RNA sequence encoded by SEQ ID NO: 15. In some embodiments, the RNA molecule further comprises a poly-A tail.

In some embodiments, the one or more RNA molecules are self-replicating RNA molecules. In further embodiments, the one or more RNA molecules comprise a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, or 100% identity to the RNA sequence encoded by any of SEQ ID NOs:1-4.

In some aspects, provided herein are compositions comprising one or more DNA molecules encoding the one or more RNA molecules of any of the compositions disclosed herein. In some embodiments, each of the one or more DNA molecules comprises a promoter. In some embodiments, the promoter of each of the one or more DNA molecules is located 5′ of a 5′ UTR. In yet some embodiments, the promoter is a T7 promoter.

In some aspects, the present disclosure provides a composition comprising an RNA molecule comprising a sequence with at least 80% sequence identity to the RNA sequence encoded by SEQ ID NO: 5. In some embodiments, the composition further comprises a sequence with at least 80% sequence identity to the RNA sequence encoded by SEQ ID NO: 9.

In some aspects, the present disclosure provides a composition comprising an RNA molecule comprising a sequence with at least 80% sequence identify to the RNA sequence encoded by SEQ ID NO: 6. In some embodiments, the composition further comprises a sequence with at least 80% sequence identity to the RNA sequence encoded by SEQ ID NO: 10.

In some aspects, the present disclosure provides a composition comprising an RNA molecule comprising a sequence with at least 80% sequence identity to the RNA sequence encoded by SEQ ID NO: 7. In some embodiments, the composition further comprises a sequence with at least 80% sequence identity to the RNA sequence encoded by SEQ ID NO: 11.

In some aspects, the present disclosure provides a composition comprising an RNA molecule comprising a sequence with at least 80% sequence identity to the RNA sequence encoded by SEQ ID NO: 8. In some embodiments, the composition further comprises a sequence with at least 80% sequence identity to the RNA sequence encoded by SEQ ID NO: 12.

In some aspects, the present disclosure provides a composition comprising an RNA molecule comprising a sequence with at least 80% sequence identity to the RNA sequence encoded by SEQ ID NO: 9.

In some aspects, the present disclosure provides a composition comprising an RNA molecule comprising a sequence with at least 80% sequence identity to the RNA sequence encoded by SEQ ID NO: 10.

In some aspects, the present disclosure provides a composition comprising an RNA molecule comprising a sequence with at least 80% sequence identity to the RNA sequence encoded by SEQ ID NO: 11.

In some aspects, the present disclosure provides a composition comprising an RNA molecule comprising a sequence with at least 80% sequence identity to the RNA sequence encoded by SEQ ID NO: 12.

In some aspects, the present disclosure provides a composition comprising an RNA molecule comprising a sequence with at least 80% sequence identity to the RNA sequence encoded by any of SEQ ID NOs: 1-4.

In some embodiments, the compositions disclosed herein further comprise an ionizable cationic lipid. In some embodiments, the ionizable cationic lipid has a structure of Formula I:

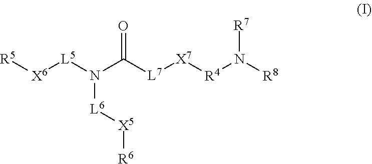

or a pharmaceutically acceptable salt or solvate thereof, wherein R⁵ and R⁶ are each independently selected from the group consisting of a linear or branched C₁-C₃₁ alkyl, C₂-C₃₁ alkenyl or C₂-C₃₁ alkynyl and cholesteryl; L⁵ and L⁶ are each independently selected from the group consisting of a linear C₁-C₂₀ alkyl and C₂-C₂₀ alkenyl; X⁵ is —C(O)O—, whereby —C(O)O—R⁶ is formed or —OC(O)— whereby —OC(O)—R⁶ is formed; X⁶ is —C(O)O— whereby —C(O)O—R⁵ is formed or —OC(O)— whereby —OC(O)—R⁵ is formed; X⁷ is S or O; L⁷ is absent or lower alkyl; R⁴ is a linear or branched C₁-C₆ alkyl; and R⁷ and R⁸ are each independently selected from the group consisting of a hydrogen and a linear or branched C₁-C₆ alkyl.

In some embodiments, the ionizable cationic lipid is selected from Table 6 herein.

In some embodiments, the ionizable cationic lipid is ATX-126:

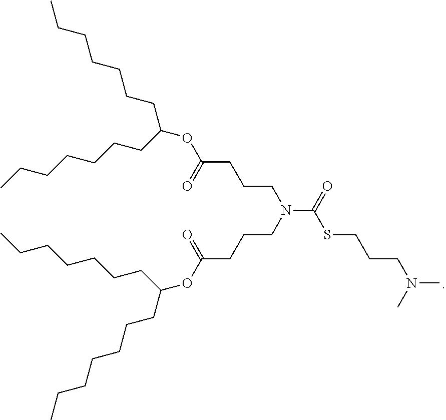 [SMILES: CCCCCCCC(CCCCCCC)OC(=O)CCCN(CCCC(=O)OC(CCCCCCC)CCCCCCC)C(=O)SCCCN(C)C]

In some embodiments, the ionizable cationic lipid is ATX-240:

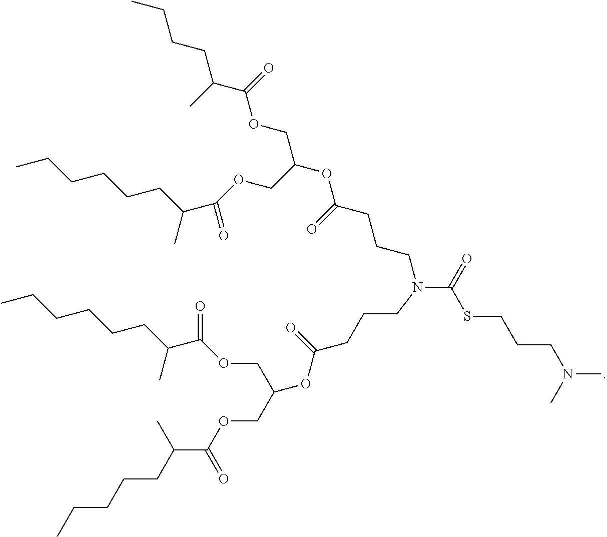 [SMILES: CCCCCCC(C)C(=O)OCC(COC(=O)C(C)CCCC)OC(=O)CCCN(CCCC(=O)OC(COC(=O)C(C)CCCCC)COC(=O)C(C)CCCCCC)C(=O)SCCCN(C)C]

In some embodiments of the compositions comprising an ionizable cationic lipd disclosed herein, the composition comprise a nitrogen to phosphate ratio (N:P) of about 5:1 To 7:1.

In one aspect, provided herein are methods of vaccinating a subject against influenza, the method comprising administering to the subject any of the compositions disclosed herein.

In one aspect, the present disclosure provides uses of any of the compositions disclosed herein in the preparation of a medicament for vaccinating a subject against influenza.

In one aspect, the present disclosure provides a composition comprising (i) a polynucleotide having a length of about 5,000 to about 20,000 nucleotides, and (ii) an ionizable cationic lipid, wherein the composition comprises a nitrogen to phosphate ratio (N:P) of about 5:1 to about 7:1. In some embodiments, the nitrogen to phosphate ratio (N:P) is about 7:1.

In some embodiments, the RNA molecules or compositions described herein are used for inducing an immune response to any of the antigens disclosed herein.

In some embodiments, the RNA molecules or compositions described herein are used in the manufacture of a medicament for inducing an immune response any of the antigens disclosed herein.

### BRIEF DESCRIPTION OF THE DRAWINGS

FIG. 1 shows schematics of various saRNA constructs for expression of a neuraminidase (NA) antigen and a hemagglutinin (HA) antigen, in accordance with some embodiments. Each of the illustrated constructs include an IRES of one or more of coxsackievirus B3 (CVB3), encephalomyocarditis virus (EMCV), or enterovirus 71 (EV71), as indicated.

FIG. 2 shows a general schematic of an exemplary quadrivalent seasonal influenza vaccine, including four separate RNAs, each encoding a replicase backbone, subgenomic promoters (sgPs), and a neuraminidase (NA) antigen and a hemagglutinin (HA) antigen from the same respective strains of influenza.

FIGS. 3A-3B show illustrative results for hemagglutinin (HA) inhibition titers in serum of vaccinated female BALB/c mice.

FIG. 4 shows illustrative results of a Meso Scale Discovery (MSD) assay depicting levels of HA-specific IgG in serum of female BALB/c mice 28 days post vaccination with an N1-pseudouridine modified mRNA vaccine encoding HA from the H3N2 A/Cambodia/e0826360/2020 strain of influenza, using two different constructions, including CODEX and hCAI codon-optimizations.

FIGS. 5A-5D show illustrative levels of HA-specific binding IgG antibodies (AU/mL) in vaccinated female BALB/c mice.

FIG. 6A-6B show illustrative results for weight loss and immunogenicity in vaccinated female BALB/c mice.

FIG. 7A-7H show illustrative results for tolerability and/or immunogenicity in animals vaccinated with various vaccine compositions.

FIG. 8A-8H show illustrative results for the immunogenicity of vaccines with varying designs.

FIGS. 9A-9D show illustrative results for immunogenicity of vaccines with varying designs.

### DETAILED DESCRIPTION

The present disclosure relates to RNAs, e.g., self-replicating RNAs and messenger RNAs (mRNAs), and nucleic acids encoding the same for expression of transgenes such as antigenic proteins, for example. Also provided herein are methods of administration (e.g., to a host, such as a mammalian subject) of RNAs, whereby the RNA is translated in vivo and the heterologous protein-coding sequence is expressed and, e.g., can elicit an immune response to the heterologous protein-coding sequence in the recipient or provide a therapeutic effect, including induction of an immune response, where the heterologous protein-coding sequence is a therapeutic or an antigenic protein. RNAs, e.g., self-replicating RNAs and messenger RNAs (mRNAs), provided herein are useful as vaccines that can be rapidly generated and that can be effective at low and/or single doses. The present disclosure further relates to methods of inducing an immune response using RNAs provided herein.

In some embodiments, an immune response can be elicited against influenza. Immunogens include, but are not limited to, those derived from type A influenza strains (such as H1N1 or H3N2) or type B influenza strains (such as Victoria or Yamagata).

Self-replicating RNAs are described, for example, in U.S. 2018/0036398, the contents of which are incorporated herein by reference in their entirety.

### Definitions

As used herein, the term “fragment,” when referring to a protein or nucleic acid, for example, means any shorter sequence than the full-length protein or nucleic acid. Accordingly, any sequence of a nucleic acid or protein other than the full-length nucleic acid or protein sequence can be a fragment. In some aspects, a protein fragment includes an epitope. In other aspects, a protein fragment is an epitope.

As used herein, the term “nucleic acid” refers to any deoxyribonucleic acid (DNA) molecule, ribonucleic acid (RNA) molecule, or nucleic acid analogues. A DNA or RNA molecule can be double-stranded or single-stranded and can be of any size. Exemplary nucleic acids include, but are not limited to, chromosomal DNA, plasmid DNA, cDNA, cell-free DNA (cfDNA), mitochondrial DNA, chloroplast DNA, viral DNA, mRNA, tRNA, rRNA, long non-coding RNA, siRNA, micro RNA (miRNA or miR), hnRNA, and viral RNA. Exemplary nucleic analogues include peptide nucleic acid, morpholino- and locked nucleic acid, glycol nucleic acid, and threose nucleic acid. As used herein, the term “nucleic acid molecule” is meant to include fragments of nucleic acid molecules as well as any full-length or non-fragmented nucleic acid molecule, for example. As used herein, the terms “nucleic acid” and “nucleic acid molecule” can be used interchangeably, unless context clearly indicates otherwise.

As used herein, the term “polynucleotide” refers to a nucleic acid sequence that includes at least two nucleotide monomers. The term “polynucleotide” can refer to polymers of DNA, RNA, nucleic acid analogues, or combinations of these. A “polynucleotide” can be double-stranded or single-stranded and can be of any size. A polynucleotide can be a separate nucleic acid molecule or be a part of a nucleic acid molecule. Accordingly, the term “polynucleotide” can refer to a nucleic acid molecule or to a region of a nucleic acid molecule.

As used herein, the term “protein” refers to any polymeric chain of amino acids. The terms “peptide” and “polypeptide” can be used interchangeably with the term protein, unless context clearly indicates otherwise, and can also refer to a polymeric chain of amino acids. The term “protein” encompasses native or artificial proteins, protein fragments and polypeptide analogs of a protein sequence. A protein may be monomeric or polymeric. The term “protein” encompasses fragments and variants (including fragments of variants) thereof, unless otherwise contradicted by context.

In general, “sequence identity” or “sequence homology,” which can be used interchangeably, refer to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby or the amino acid sequence of a polypeptide, and comparing these sequences to a second nucleotide or amino acid sequence. As used herein, the term “percent (%) sequence identity” or “percent (%) identity,” also including “percent homology,” refers to the percentage of amino acid residues or nucleotides in a sequence that are identical with the amino acid residues or nucleotides in a reference sequence after aligning the sequences and introducing gaps, if necessary, to achieve the maximum percent sequence identity, and not considering any conservative substitutions as part of the sequence identity. Thus, two or more sequences (polynucleotide or amino acid) can be compared by determining their “percent identity,” also referred to as “percent homology.” The percent identity to a reference sequence (e.g., nucleic acid or amino acid sequences), which may be a sequence within a longer molecule (e.g., polynucleotide or polypeptide), may be calculated as the number of exact matches between two optimally aligned sequences divided by the length of the reference sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul et al., J. Mol. Biol. 215:403-410 (1990); Karlin and Altschul, Proc. Natl. Acad. sci. USA 90:5873-5877 (1993); and Altschul et al., Nucleic Acids Res. 25:3389-3402 (1997). Briefly, the BLAST program defines identity as the number of identical aligned symbols (i.e., nucleotides or amino acids), divided by the total number of symbols in the shorter of the two sequences. The program may be used to determine percent identity over the entire length of the sequences being compared. Default parameters are provided to optimize searches with short query sequences, for example, with the blastp program. The program also allows use of an SEG filter to mask-off segments of the query sequences as determined by the SEG program of Wootton and Federhen, Computers and Chemistry 17: 149-163 (1993). Ranges of desired degrees of sequence identity are approximately 80% to 100% and integer values in between. Percent identities between a reference sequence and a claimed sequence can be at least 80%, at least 85%, at least 90%, at least 95%, at least 98%, at least 99%, at least 99.5%, or at least 99.9%. In general, an exact match indicates 100% identity over the length of the reference sequence. Additional programs and methods for comparing sequences and/or assessing sequence identity include the Needleman-Wunsch algorithm (see, e.g., the EMBOSS Needle aligner available at ebi.ac.uk/Tools/psa/emboss needle/, optionally with default settings), the Smith-Waterman algorithm (see, e.g., the EMBOSS Water aligner available at ebi.ac.uk/Tools/psa/emboss water/, optionally with default settings), the similarity search method of Pearson and Lipman, 1988, Proc. Natl. Acad. Sci. USA 85, 2444, or computer programs which use these algorithms (GAP, BESTFIT, FASTA, BLAST P, BLAST N and TFASTA in Wisconsin Genetics Software Package, Genetics Computer Group. 575 Science Drive, Madison, Wis.). In some aspects, reference to percent sequence identity refers to sequence identity as measured using BLAST (Basic Local Alignment Search Tool). In other aspects, ClustalW is used for multiple sequence alignment. Optimal alignment may be assessed using any suitable parameters of a chosen algorithm, including default parameters.

As used herein, “homologous sequences” refers to sequences that share sequence similarity and/or structural similarity (Pearson, 2013, An Introduction to Sequence similarity (“Homology”) Searching, Current Protoc Bioinformatics, 42:3.1.1-3.1.8). Accordingly, homologous sequences share common evolutionary ancestry or are derived from a common sequence. Homologous sequences can also share structural or sequence similarity to an intermediate sequence. Homologous sequences can have similar functions, i.e., have functional similarity. Homology can be inferred based on nucleic acid and/or amino acid sequence, with protein similarity searches generally having greater sensitivity than nucleic acid sequence searches. Homology can also be inferred for amino acid sequences that include similar amino acids, i.e., amino acids with similar physiochemical properties, rather than identical amino acids over at least a region of sequence. The terms “homologous sequences,” “homologues,” and “homologous nucleic acid” and/or “homologous protein” can be used interchangeably, unless context clearly indicated otherwise.

As used herein, the term “drug” or “medicament,” means a pharmaceutical formulation or composition as described herein.

As used herein, the singular forms “a,” “an,” and “the” include plural references unless the context clearly dictates otherwise. Thus, for example, references to “the method” includes one or more methods, and/or steps of the type described herein which will become apparent to those persons skilled in the art upon reading this disclosure and so forth.

“About” as used herein when referring to a measurable value such as an amount, a temporal duration, and the like, is meant to encompass variations of +20%, or ±10%, or ±5%, or even ±1% from the specified value, as such variations are appropriate for the disclosed methods or to perform the disclosed methods.

The term “expression” refers to the process by which a nucleic acid sequence or a polynucleotide is transcribed from a DNA template (such as into mRNA or other RNA transcript) and/or the process by which a transcribed mRNA or other RNA is subsequently translated into peptides, polypeptides, or proteins. Transcripts and encoded polypeptides may be collectively referred to as “gene product.”

As used herein, the terms “self-replicating RNA,” “self-transcribing and self-replicating RNA,” “self-amplifying RNA” (saRNA or samRNA), and “replicon” may be used interchangeably, unless context clearly indicates otherwise. Generally, the term “replicon” or “viral replicon” refers to a self-replicating subgenomic RNA derived from a viral genome that includes viral genes encoding non-structural proteins important for viral replication and that lacks viral genes encoding structural proteins. A self-replicating RNA can encode further subgenomic RNAs that are not able to self-replicate. A self-replicating RNA can also be referred to as a “STARR™” RNA.

As used herein, “operably linked,” “operable linkage,” “operatively linked,” or grammatical equivalents thereof refer to juxtaposition of genetic elements, e.g., a promoter, an enhancer, a polyadenylation sequence, etc., wherein the elements are in a relationship permitting them to operate in the expected manner. For instance, a regulatory element, which can comprise promoter and/or enhancer sequences, is operatively linked to a coding region if the regulatory element helps initiate transcription of the coding sequence. There may be intervening residues between the regulatory element and coding region so long as this functional relationship is maintained.

### RNA Molecules

In some aspects, provided herein are RNA molecules collectively encoding a hemagglutinin (HA) polypeptide and a neuraminidase (NA) polypeptide of each of four different strains of influenza virus, and polynucleotides encoding the RNA molecules. In some embodiments, the one or more RNA molecules comprise 8 RNA molecules (e.g., one for each NA and one for each HA). In some embodiments, two or more influenza polypeptides (e.g., two HA, two NA, or an HA and an NA) are encoded on a single RNA molecule, such that fewer than 8 RNA molecules encode the HA and NA polypeptides of the four different strains (e.g., 7, 6, 5, 4, 3, or 2 RNA molecules). In some embodiments, the HA polypeptide and the NA polypeptide are encoded by the same RNA molecule for each of the four strains of influenza virus. In some embodiments, the HA polypeptide and the NA polypeptide are encoded by the same RNA molecule for each of the four different strains of influenza.

Also provided herein are RNA molecules for expressing an antigen comprising a 5′ UTR comprising the sequence of SEQ ID NO:14, and a 3′ UTR comprising the sequence of SEQ ID NO:15; or a 5′ UTR comprising the sequence of SEQ ID NO:17, and a 3′ UTR comprising the sequence of SEQ ID NO:19, wherein T is substituted with U.

In some embodiments, the RNA molecule includes a poly-A tail. The poly-A tail may be transcribed directly from a template polynucleotide, or may be added post-transcriptionally. The poly-A tail may have any of a variety of lengths, such as 20-300 nucleotides. In some embodiments, the poly-A tail is about 130 nucleotides in length. In some embodiments, the poly-A tail is encoded by a polynucleotide having the sequence of SEQ ID NO: 16. In some embodiments, the poly-A tail is encoded by a polynucleotide having the sequence of nucleotides 31-130 of SEQ ID NO: 16. In some embodiments, the poly-A tail is less than 100 nucleotides in length (e.g., 20-99 nucleotides in length). In some embodiments, the poly-A tail is less than 90, 80, 75, 70, 65, 60, 55, or 50 nucleotides in length.

An RNA molecule can encode a single polypeptide immunogen or multiple polypeptides. Multiple immunogens can be presented as a single polypeptide immunogen (fusion polypeptide) or as separate polypeptides. If immunogens are expressed as separate polypeptides from a replicon, then one or more of these may be provided with an upstream IRES or an additional viral promoter element. Alternatively, multiple immunogens may be expressed from a polyprotein that encodes individual immunogens fused to a short autocatalytic protease (e.g. foot-and-mouth disease virus 2A protein), or as inteins.

In some aspects, provided herein are compositions comprising one or more RNA molecules, wherein the one or more RNA molecules collectively encode a hemagglutinin (HA) polypeptide and a neuraminidase (NA) polypeptide of each of four different strains of influenza virus. In some embodiments, the four different strains of influenza virus comprise one or more of H1N1, H3N2, Victoria-B, or Yamagata-B. In further embodiments, the four different strains of influenza virus comprise Victoria B/Austria/1359417/2021, H3N2 A/Darwin/6/2021, H1N1 A/Wisconsin/588/2019, and Yamagata B/PHUKET/3073/2013.

In some embodiments, the HA polypeptide and the NA polypeptide are encoded by the same RNA molecule for each of the four different strains of influenza. In some aspects, the HA and NA polypeptides of a first strain of influenza virus are encoded by a first RNA molecule, the HA and NA polypeptides of a second strain of influenza virus are encoded by a second RNA molecule, the HA and NA polypeptides of a third strain of influenza virus are encoded by a third RNA molecule, and the HA and NA polypeptides of a fourth strain of influenza virus are encoded by a fourth RNA molecule. In some embodiments, the first, second, third, and fourth RNA molecules are present in an equimolar ratio. In some embodiments, each of the one or more RNA molecules further encodes one or more viral replication proteins. In some embodiments, the one or more viral replication proteins are alphavirus proteins. In yet further embodiments, the alphavirus proteins are from Venezuelan Equine Encephalitis Virus (VEEV).

In one aspect, each of the one or more RNA molecules of any of the compositions disclosed herein encodes in a 5′ to 3′ order: (i) the one or more viral replication proteins, (ii) one of the NA polypeptides; and (iii) one of the HA polypeptides. In some embodiments, the one or more viral replication proteins comprise an alphavirus nonstructural protein 1 (nsP1), an alphavirus nonstructural protein 2 (nsP2), an alphavirus nonstructural protein 3 (nsP3), an alphavirus nonstructural protein 4 (nsP4), or any combination thereof. In further embodiments, the one or more viral replication proteins comprises a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, or 100% identity to the RNA sequence encoded by SEQ ID NO: 13.

In some aspects, the sequences encoding the HA and NA polypeptides of at least one of the one or more RNA molecules are preceded by a subgenomic promoter (sgP).

In one aspect, each HA polypeptide of any of the compositions disclosed herein comprises an antigenic fragment of a respective HA protein. In another aspect, each NA polypeptide of any of the compositions disclosed herein comprises an antigenic fragment of a respective NA protein.

In some embodiments, each of the one or more RNA molecules of any of the compositions disclosed herein further comprise a 5′ untranslated region (UTR). In further embodiments, at least one 5′ UTR comprises a viral 5′ UTR, a non-viral 5′ UTR, or a combination of viral and non-viral 5′ UTR sequences. In yet further embodiments, the at least one 5′ UTR comprises an alphavirus 5′ UTR. In still further embodiments, the alphavirus 5′ UTR comprises a Venezuelan Equine Encephalitis Virus (VEEV) 5′ UTR sequence.

In some embodiments, at least one 5′ UTR of any of the compositions disclosed herein comprises the RNA sequence encoded by SEQ ID NO: 14.

In some embodiments, each of the one or more RNA molecules of any of the compositions disclosed herein further comprise a 3′ untranslated region (UTR). In some embodiments, at least one 3′ UTR comprises a viral 3′ UTR, a non-viral 3′ UTR, or a combination of viral and non-viral 3′ UTR sequences. In further embodiments, the at least one 3′ UTR comprises an alphavirus 3′ UTR sequence. In yet further embodiments, the alphavirus 3′ UTR comprises a Venezuelan Equine Encephalitis Virus (VEEV) 3′ UTR sequence. In still further embodiments, at least one 3′UTR comprises the RNA sequence encoded by SEQ ID NO: 15.

In some embodiments, the RNA molecule of any of the compositions disclosed herein further comprise a poly-A tail. The poly-A tail may be transcribed directly from a template polynucleotide, or may be added post-transcriptionally. The poly-A tail may have any of a variety of lengths, such as 20-300 nucleotides. In some embodiments, the poly-A tail is about 130 nucleotides in length. In some embodiments, the poly-A tail is encoded by a polynucleotide having the sequence of SEQ ID NO: 16. In some embodiments, the poly-A tail is encoded by a polynucleotide having the sequence of nucleotides 31-130 of SEQ ID NO: 16. In some embodiments, the poly-A tail is less than 100 nucleotides in length (e.g., 20-99 nucleotides in length). In some embodiments, the poly-A tail is less than 90, 80, 75, 70, 65, 60, 55, or 50 nucleotides in length.

In some aspects, the RNA molecules disclosed herein are self-replicating RNA molecules. In some embodiments, the RNA molecules comprise a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, or 100% identity to the RNA sequence encoded by any of SEQ ID NOs:1-4. In some embodiments, the RNA molecules comprise a first RNA molecule having at least 80% sequence identity (e.g., at least 85%, 90%, 91%, 92%, 93%, 94%, 95%, 96%, 97%, 98%, 99% sequence identity) to the RNA sequence encoded by SEQ ID NO: 1. In some embodiments, the first RNA molecule has at least 90% sequence identity to the RNA sequence encoded by SEQ ID NO: 1. In some embodiments, the first RNA molecule has at least 95% sequence identity to the RNA sequence encoded by SEQ ID NO: 1. In some embodiments, the first RNA molecule comprises the RNA sequence encoded by SEQ ID NO: 1. In some embodiments, the RNA molecules comprise a second RNA molecule having at least 80% sequence identity (e.g., at least 85%, 90%, 91%, 92%, 93%, 94%, 95%, 96%, 97%, 98%, 99% sequence identity) to the RNA sequence encoded by SEQ ID NO: 2. In some embodiments, the second RNA molecule has at least 90% sequence identity to the RNA sequence encoded by SEQ ID NO: 2. In some embodiments, the second RNA molecule has at least 95% sequence identity to the RNA sequence encoded by SEQ ID NO: 2. In some embodiments, the second RNA molecule comprises the RNA sequence encoded by SEQ ID NO: 2. In some embodiments, the RNA molecules comprise a third RNA molecule having at least 80% sequence identity (e.g., at least 85%, 90%, 91%, 92%, 93%, 94%, 95%, 96%, 97%, 98%, 99% sequence identity) to the RNA sequence encoded by SEQ ID NO: 3. In some embodiments, the third RNA molecule has at least 90% sequence identity to the RNA sequence encoded by SEQ ID NO: 3. In some embodiments, the third RNA molecule has at least 95% sequence identity to the RNA sequence encoded by SEQ ID NO: 3. In some embodiments, the third RNA molecule comprises the RNA sequence encoded by SEQ ID NO: 3. In some embodiments, the RNA molecules comprise a fourth RNA molecule having at least 80% sequence identity (e.g., at least 85%, 90%, 91%, 92%, 93%, 94%, 95%, 96%, 97%, 98%, 99% sequence identity) to the RNA sequence encoded by SEQ ID NO: 4. In some embodiments, the fourth RNA molecule has at least 90% sequence identity to the RNA sequence encoded by SEQ ID NO: 4. In some embodiments, the fourth RNA molecule has at least 95% sequence identity to the RNA sequence encoded by SEQ ID NO: 4. In some embodiments, the fourth RNA molecule comprises the RNA sequence encoded by SEQ ID NO: 4. In some embodiments, a composition provided herein comprises a combination of the first, second, third, and fourth RNA molecule. In some embodiments, the composition comprises a first RNA molecule encoded by SEQ ID NO: 1, a second RNA molecule encoded by SEQ ID NO: 2, a third RNA molecule encoded by SEQ ID NO: 3, and a fourth RNA molecule encoded by SEQ ID NO: 4, each of which may comprise a poly-A tail encoded by SEQ ID NO: 16 at their 3′ ends. In some embodiments, the first, second, third, and fourth RNA molecules are present in the combination in equimolar amounts. In some embodiments, the poly-A-encoding sequence in one or more of (e.g., all of) SEQ ID NOs: 1-4 is replaced with another sequence encoding a poly-A tail described herein.

In some embodiments, the compositions disclosed herein comprise DNA molecules encoding the RNA molecules of any of the compositions disclosed herein. In some embodiments, each of the DNA molecules comprise a promoter. In further embodiments, the promoter of each DNA molecule is located 5′ of a 5′ UTR. In yet further embodiments, the promoter is a T7 promoter.

In some aspects, the present disclosure provides a composition comprising an RNA molecule comprising a sequence with at least 80% sequence identity (e.g., at least 85%, 90%, 91%, 92%, 93%, 94%, 95%, 96%, 97%, 98%, 99% sequence identity) to the RNA sequence encoded by SEQ ID NO: 5. In some embodiments, the RNA molecule comprises a sequence with at least 90% sequence identity to the RNA sequence encoded by SEQ ID NO: 5. In some embodiments, the RNA molecule comprises a sequence with at least 95% sequence identity to the RNA sequence encoded by SEQ ID NO: 5. In some embodiments, the RNA molecule comprises the RNA sequence encoded by SEQ ID NO: 5. In some embodiments, the composition further comprises a sequence with at least 80% sequence identity (e.g., at least 85%, 90%, 91%, 92%, 93%, 94%, 95%, 96%, 97%, 98%, 99% sequence identity) to the RNA sequence encoded by SEQ ID NO: 9, which may be present on the same or different RNA molecule. In some embodiments, the further sequence is at least 90% identical to the RNA sequence encoded by SEQ ID NO: 9. In some embodiments, the further sequence is at least 95% identical to the RNA sequence encoded by SEQ ID NO: 9. In some embodiments, the further sequence is the RNA sequence encoded by SEQ ID NO: 9.

In some aspects, the present disclosure provides a composition comprising an RNA molecule comprising a sequence with at least 80% sequence identity (e.g., at least 85%, 90%, 91%, 92%, 93%, 94%, 95%, 96%, 97%, 98%, 99% sequence identity) to the RNA sequence encoded by SEQ ID NO: 6. In some embodiments, the RNA molecule comprises a sequence with at least 90% sequence identity to the RNA sequence encoded by SEQ ID NO: 6. In some embodiments, the RNA molecule comprises a sequence with at least 95% sequence identity to the RNA sequence encoded by SEQ ID NO: 6. In some embodiments, the RNA molecule comprises the RNA sequence encoded by SEQ ID NO: 6. In some embodiments, the composition further comprises a sequence with at least 80% sequence identity (e.g., at least 85%, 90%, 91%, 92%, 93%, 94%, 95%, 96%, 97%, 98%, 99% sequence identity) to the RNA sequence encoded by SEQ ID NO: 10, which may be present on the same or different RNA molecule. In some embodiments, the further sequence is at least 90% identical to the RNA sequence encoded by SEQ ID NO: 10. In some embodiments, the further sequence is at least 95% identical to the RNA sequence encoded by SEQ ID NO: 10. In some embodiments, the further sequence is the RNA sequence encoded by SEQ ID NO: 10.

In some aspects, the present disclosure provides a composition comprising an RNA molecule comprising a sequence with at least 80% sequence identity (e.g., at least 85%, 90%, 91%, 92%, 93%, 94%, 95%, 96%, 97%, 98%, 99% sequence identity) to the RNA sequence encoded by SEQ ID NO: 7. In some embodiments, the RNA molecule comprises a sequence with at least 90% sequence identity to the RNA sequence encoded by SEQ ID NO: 7. In some embodiments, the RNA molecule comprises a sequence with at least 95% sequence identity to the RNA sequence encoded by SEQ ID NO: 7. In some embodiments, the RNA molecule comprises the RNA sequence encoded by SEQ ID NO: 7. In some embodiments, the composition further comprises a sequence with at least 80% sequence identity (e.g., at least 85%, 90%, 91%, 92%, 93%, 94%, 95%, 96%, 97%, 98%, 99% sequence identity) to the RNA sequence encoded by SEQ ID NO: 11, which may be present on the same or different RNA molecule. In some embodiments, the further sequence is at least 90% identical to the RNA sequence encoded by SEQ ID NO: 11. In some embodiments, the further sequence is at least 95% identical to the RNA sequence encoded by SEQ ID NO: 11. In some embodiments, the further sequence is the RNA sequence encoded by SEQ ID NO: 11.

In some aspects, the present disclosure provides a composition comprising an RNA molecule comprising a sequence with at least 80% sequence identity (e.g., at least 85%, 90%, 91%, 92%, 93%, 94%, 95%, 96%, 97%, 98%, 99% sequence identity) to the RNA sequence encoded by SEQ ID NO: 8. In some embodiments, the RNA molecule comprises a sequence with at least 90% sequence identity to the RNA sequence encoded by SEQ ID NO: 8. In some embodiments, the RNA molecule comprises a sequence with at least 95% sequence identity to the RNA sequence encoded by SEQ ID NO: 8. In some embodiments, the RNA molecule comprises the RNA sequence encoded by SEQ ID NO: 8. In some embodiments, the composition further comprises a sequence with at least 80% sequence identity (e.g., at least 85%, 90%, 91%, 92%, 93%, 94%, 95%, 96%, 97%, 98%, 99% sequence identity) to the RNA sequence encoded by SEQ ID NO: 12, which may be present on the same or different RNA molecule. In some embodiments, the further sequence is at least 90% identical to the RNA sequence encoded by SEQ ID NO: 12. In some embodiments, the further sequence is at least 95% identical to the RNA sequence encoded by SEQ ID NO: 12. In some embodiments, the further sequence is the RNA sequence encoded by SEQ ID NO: 12.

In some aspects, the present disclosure provides a composition comprising an RNA molecule comprising a sequence with at least 80% sequence identity (e.g., at least 85%, 90%, 91%, 92%, 93%, 94%, 95%, 96%, 97%, 98%, 99% sequence identity) to the RNA sequence encoded by SEQ ID NO: 9. In some embodiments, the RNA molecule comprises a sequence with at least 90% sequence identity to the RNA sequence encoded by SEQ ID NO: 9. In some embodiments, the RNA molecule comprises a sequence with at least 95% sequence identity to the RNA sequence encoded by SEQ ID NO: 9. In some embodiments, the RNA molecule comprises the RNA sequence encoded by SEQ ID NO: 9.

In some aspects, the present disclosure provides a composition comprising an RNA molecule comprising a sequence with at least 80% sequence identity (e.g., at least 85%, 90%, 91%, 92%, 93%, 94%, 95%, 96%, 97%, 98%, 99% sequence identity) to the RNA sequence encoded by SEQ ID NO: 10. In some embodiments, the RNA molecule comprises a sequence with at least 90% sequence identity to the RNA sequence encoded by SEQ ID NO: 10. In some embodiments, the RNA molecule comprises a sequence with at least 95% sequence identity to the RNA sequence encoded by SEQ ID NO: 10. In some embodiments, the RNA molecule comprises the RNA sequence encoded by SEQ ID NO: 10.

In some aspects, the present disclosure provides a composition comprising an RNA molecule comprising a sequence with at least 80% sequence identity (e.g., at least 85%, 90%, 91%, 92%, 93%, 94%, 95%, 96%, 97%, 98%, 99% sequence identity) to the RNA sequence encoded by SEQ ID NO: 11. In some embodiments, the RNA molecule comprises a sequence with at least 90% sequence identity to the RNA sequence encoded by SEQ ID NO: 11. In some embodiments, the RNA molecule comprises a sequence with at least 95% sequence identity to the RNA sequence encoded by SEQ ID NO: 11. In some embodiments, the RNA molecule comprises the RNA sequence encoded by SEQ ID NO: 11.

In some aspects, the present disclosure provides a composition comprising an RNA molecule comprising a sequence with at least 80% sequence identity (e.g., at least 85%, 90%, 91%, 92%, 93%, 94%, 95%, 96%, 97%, 98%, 99% sequence identity) to the RNA sequence encoded by SEQ ID NO: 12. In some embodiments, the RNA molecule comprises a sequence with at least 90% sequence identity to the RNA sequence encoded by SEQ ID NO: 12. In some embodiments, the RNA molecule comprises a sequence with at least 95% sequence identity to the RNA sequence encoded by SEQ ID NO: 12. In some embodiments, the RNA molecule comprises the RNA sequence encoded by SEQ ID NO: 12.

In some aspects, the RNA molecules disclosed herein are self-replicating RNA molecules. In some embodiments, the RNA molecules comprise a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, or 100% identity to the RNA sequence encoded by any of SEQ ID NOs:151-161. In some embodiments, the RNA molecules comprise a first RNA molecule having at least 80% sequence identity (e.g., at least 85%, 90%, 91%, 92%, 93%, 94%, 95%, 96%, 97%, 98%, 99% sequence identity) to the RNA sequence encoded by any one of SEQ ID NOs: 151-161. In some embodiments, the first RNA molecule has at least 90% sequence identity to the RNA sequence encoded by any one of SEQ ID NOs: 151-161. In some embodiments, the first RNA molecule has at least 95% sequence identity to the RNA sequence encoded by any one of SEQ ID NOs: 151-161. In some embodiments, the first RNA molecule comprises the RNA sequence encoded by any one of SEQ ID NOs: 151-161.

### Codon Optimization

In some embodiments, compositions provided herein encode one or more viral replication proteins include codon-optimized sequences. As a result, protein-coding sequences provided herein may be substantially varied and still encode the same protein. As used herein, the term “codon-optimized” means a polynucleotide, nucleic acid sequence, or coding sequence has been redesigned as compared to a wild-type or reference polynucleotide, nucleic acid sequence, or coding sequence by choosing different codons without altering the amino acid sequence of the encoded protein. Accordingly, codon-optimization generally refers to replacement of codons with synonymous codons to optimize expression of a protein while keeping the amino acid sequence of the translated protein the same. Codon optimization of a sequence can increase protein expression levels (Gustafsson et al., Codon bias and heterologous protein expression. 2004, Trends Biotechnol 22: 346-53) of the encoded proteins, for example, and provide other advantages. Variables such as codon usage preference as measured by codon adaptation index (CAI), for example, the presence or frequency of U and other nucleotides, mRNA secondary structures, cis-regulatory sequences, GC content, and other variables may correlate with protein expression levels (Villalobos et al., Gene Designer: a synthetic biology tool for constructing artificial DNA segments. 2006, BMC Bioinformatics 7:285). Polynucleotides can be codon-optimized before modifying miRNA binding sites. miRNA binding sites can be modified to replace one or more codons with synonymous codons.

Any method of codon optimization can be used to codon optimize polynucleotides and nucleic acid molecules provided herein, and any variable can be altered by codon optimization. Accordingly, any combination of codon optimization methods can be used. Exemplary methods include the high codon adaptation index (CAI) method, the Low U method, and others. The CAI method chooses a most frequently used synonymous codon for an entire protein coding sequence. As an example, the most frequently used codon for each amino acid can be deduced from 74,218 protein-coding genes from a human genome. The Low U method targets U-containing codons that can be replaced with a synonymous codon with fewer U moieties, generally without changing other codons. If there is more than one choice for replacement, the more frequently used codon can be selected. Any polynucleotide, nucleic acid sequence, or codon sequence provided herein can be codon-optimized.

In some embodiments, the nucleotide sequence of any region of the RNA or DNA templates described herein may be codon optimized, including, for example, nucleotide sequences encoding viral replication proteins, hemagglutinin proteins, neuraminidase proteins, or any combination thereof. Preferably, the primary cDNA template may include reducing the occurrence or frequency of appearance of certain nucleotides in the template strand. For example, the occurrence of a nucleotide in a template may be reduced to a level below 25% of said nucleotides in the template. In further examples, the occurrence of a nucleotide in a template may be reduced to a level below 20% of said nucleotides in the template. In some examples, the occurrence of a nucleotide in a template may be reduced to a level below 16% of said nucleotides in the template. Preferably, the occurrence of a nucleotide in a template may be reduced to a level below 15%, and preferably may be reduced to a level below 12% of said nucleotides in the template.

In some embodiments, the nucleotide reduced is uridine. For example, the present disclosure provides nucleic acids with altered uracil content wherein at least one codon in the wild-type sequence has been replaced with an alternative codon to generate a uracil-altered sequence. Altered uracil sequences can have at least one of the following properties:
(i) an increase or decrease in global uracil content (i.e., the percentage of uracil of the total nucleotide content in the nucleic acid of a section of the nucleic acid, e.g., the open reading frame);
(ii) an increase or decrease in local uracil content (i.e., changes in uracil content are limited to specific subsequences);
(iii) a change in uracil distribution without a change in the global uracil content;
(iv) a change in uracil clustering (e.g., number of clusters, location of clusters, or distance between clusters); or
(v) combinations thereof.

In some embodiments, the percentage of uracil nucleobases in the nucleic acid sequence is reduced with respect to the percentage of uracil nucleobases in the wild-type nucleic acid sequence. For example, 30% of nucleobases may be uracil in the wild-type sequence but the nucleobases that are uracil are preferably lower than 15%, preferably lower than 12% and preferably lower than 10% of the nucleobases in the nucleic acid sequences of the disclosure. The percentage uracil content can be determined by dividing the number of uracil in a sequence by the total number of nucleotides and multiplying by 100.

In some embodiments, the percentage of uracil nucleobases in a subsequence of the nucleic acid sequence is reduced with respect to the percentage of uracil nucleobases in the corresponding subsequence of the wild-type sequence. For example, the wild-type sequence may have a 5′-end region (e.g., 30 codons) with a local uracil content of 30%, and the uracil content in that same region could be reduced to preferably 15% or lower, preferably 12% or lower and preferably 10% or lower in the nucleic acid sequences of the disclosure. These subsequences can also be part of the wild-type sequences of the heterologous 5′ and 3′ UTR sequences of the present disclosure.

In some embodiments, codons in the nucleic acid sequence of the disclosure reduce or modify, for example, the number, size, location, or distribution of uracil clusters that could have deleterious effects on protein translation. Although lower uracil content is desirable in certain aspects, the uracil content, and in particular the local uracil content, of some subsequences of the wild-type sequence can be greater than the wild-type sequence and still maintain beneficial features (e.g., increased expression).

In some embodiments, the uracil-modified sequence induces a lower Toll-Like Receptor (TLR) response when compared to the wild-type sequence. Several TLRs recognize and respond to nucleic acids. Double-stranded (ds)RNA, a frequent viral constituent, has been shown to activate TLR3. Single-stranded (ss)RNA activates TLR7. RNA oligonucleotides, for example RNA with phosphorothioate internucleotide linkages, are ligands of human TLR8. DNA containing unmethylated CpG motifs, characteristic of bacterial and viral DNA, activate TLR9.

As used herein, the term “TLR response” is defined as the recognition of single-stranded RNA by a TLR7 receptor, and preferably encompasses the degradation of the RNA and/or physiological responses caused by the recognition of the single-stranded RNA by the receptor. Methods to determine and quantify the binding of an RNA to a TLR7 are known in the art. Similarly, methods to determine whether an RNA has triggered a TLR7-mediated physiological response (e.g., cytokine secretion) are well known in the art. In some embodiments, a TLR response can be mediated by TLR3, TLR8, or TLR9 instead of TLR7. Suppression of TLR7-mediated response can be accomplished via nucleoside modification. RNA undergoes over a hundred different nucleoside modifications in nature. Human rRNA, for example, has ten times more pseudouracil (′P) and 25 times more 2′-O-methylated nucleosides than bacterial rRNA. Bacterial RNA contains no nucleoside modifications, whereas mammalian RNAs have modified nucleosides such as 5-methylcytidine (m5C), N6-methyladenosine (m6A), inosine and many 2′-O-methylated nucleosides in addition to N7-methylguanosine (m7G).

In some embodiments, the uracil content of polynucleotides disclosed herein is less than about 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% of the total nucleobases in the sequence in the reference sequence. In some embodiments, the uracil content of polynucleotides disclosed herein is between about 5% and about 25%. In some embodiments, the uracil content of polynucleotides disclosed herein is between about 15% and about 25%.

In some embodiments, the nucleotide that is increased or decreased is a nucleotide other than or in addition to uracil. Sequences with altered nucleotide content can have (i) an increase or decrease in local C content (i.e., changes in cytosine content are limited to specific subsequences); (ii) an increase or decrease in local G content (i.e., changes in guanosine content are limited to specific subsequences); or (iii) a combination thereof.

In some embodiments, polynucleotides of nucleic acid molecules provided herein comprise a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to the sequence of SEQ ID NO:13. In some embodiments, polynucleotides of nucleic acid molecules provided herein comprise the sequence of SEQ ID NO:13.

### Intergenic Region

In some embodiments, polynucleotides provided herein (e.g., polynucleotides encoding an NA polypeptide and polynucleotides ending an HA polypeptide) are included in the same (i.e., a single) or in separate nucleic acid molecules. In some embodiments, polynucleotides of nucleic acid molecules provided herein are included in a single nucleic acid molecule. In some embodiments, a polynucleotide encoding an NA polypeptide is located 5′ of a polynucleotide encoding an HA polypeptide. In some embodiments, polynucleotides encoding NA and HA polypeptides are included in separate nucleic acid molecules.

In some embodiments, a first polynucleotide (e.g., a polynucleotide that encodes one or more viral replication proteins, or a polynucleotide encoding an NA polypeptide) and a second polynucleotide (e.g., a polynucleotide encoding an NA polypeptide or an HA polypeptide) are included in the same (i.e., a single) nucleic acid molecule. In some embodiments, a nucleic acid molecule herein includes first, second, and third polynucleotides (e.g., a polynucleotide encoding one or more viral replication proteins, a polynucleotide encoding an NA polypeptide, and a polynucleotide encoding an HA polypeptide). Sequentially located polynucleotides of nucleic acid molecules provided herein can be contiguous, i.e., adjacent to each other without nucleotides in between. In one aspect, an intergenic region is located between sequential polynucleotides (e.g., a first polynucleotide and a second polynucleotide, and/or between a second polynucleotide and a third polynucleotide). As used herein, the terms “intergenic region” and “intergenic sequence” can be used interchangeably, unless context clearly indicates otherwise.

An intergenic region located between polynucleotides can be of any length and can have any nucleotide sequence. As an example, the intergenic region between two polynucleotides can include about one nucleotide, about two nucleotides, about three nucleotides, about four nucleotides, about five nucleotides, about six nucleotides, about seven nucleotides, about eight nucleotides, about nine nucleotides, about ten nucleotides, about 11 nucleotides, about 12 nucleotides, about 13 nucleotides, about 14 nucleotides, about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides, about 30 nucleotides, about 31 nucleotides, about 32 nucleotides, about 33 nucleotides, about 34 nucleotides, about 35 nucleotides, about 36 nucleotides, about 37 nucleotides, about 38 nucleotides, about 39 nucleotides, about 40 nucleotides, about 41 nucleotides, about 42 nucleotides, about 43 nucleotides, about 44 nucleotides, about 45 nucleotides, about 46 nucleotides, about 47 nucleotides, about 48 nucleotides, about 49 nucleotides, about 50 nucleotides, about 60 nucleotides, about 70 nucleotides, about 80 nucleotides, about 90 nucleotides, about 100 nucleotides, about 125 nucleotides, about 150 nucleotides, about 175 nucleotides, about 200 nucleotides, about 250 nucleotides, about 300 nucleotides, about 350 nucleotides, about 400 nucleotides, about 450 nucleotides, about 500 nucleotides, about 600 nucleotides, about 700 nucleotides, about 800 nucleotides, about 900, about 1,000 nucleotides, about 1,500 nucleotides, about 2,000 nucleotides, about 2,500 nucleotides, about 3,000 nucleotides, about 3,500 nucleotides, about 4,000 nucleotides, about 4,500 nucleotides, about 5,000 nucleotides, about 6,000 nucleotides, about 7,000 nucleotides, about 8,000 nucleotides, about 9,000 nucleotides, about 10,000 nucleotides, and any number or range in between. In one aspect, the intergenic region between two polynucleotides includes about 10-100 nucleotides, about 10-200 nucleotides, about 10-300 nucleotides, about 10-400 nucleotides, or about 10-500 nucleotides. In another aspect, the intergenic region between two polynucleotides includes about 1-10 nucleotides, about 1-20 nucleotides, about 1-30 nucleotides, about 1-40 nucleotides, or about 1-50 nucleotides. In yet another aspect, the region includes about 44 nucleotides.

In one aspect, the intergenic region between two polynucleotides includes a viral sequence. The intergenic region between two polynucleotides can include a sequence from any virus, such as alphaviruses and rubiviruses, for example. In one aspect, the intergenic region between two polynucleotides comprises an alphavirus sequence, such as a sequence from Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O'Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), Buggy Creek Virus (BCRV), or any combination thereof. In another aspect, the intergenic region between two polynucleotides comprises a sequence from Venezuelan Equine Encephalitis Virus (VEEV). In yet another aspect, the intergenic region between two polynucleotides comprises a sequence having at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to the sequence of SEQ ID NO: 150. In a further aspect, the intergenic region between two polynucleotides comprises the sequence of SEQ ID NO: 150. In yet a further aspect, the intergenic region between two polynucleotides is a second intergenic region comprising a sequence having at least 85% identity to the sequence of SEQ ID NO:150.

### Natural and Modified Nucleotides

A self-replicating RNA of the disclosure can comprise one or more chemically modified nucleotides. Examples of nucleic acid monomers include non-natural, modified, and chemically-modified nucleotides, including any such nucleotides known in the art. Nucleotides can be artificially modified at either the base portion or the sugar portion. In nature, most polynucleotides comprise nucleotides that are “unmodified” or “natural” nucleotides, which include the purine bases adenine (A) and guanine (G), and the pyrimidine bases thymine (T), cytosine (C) and uracil (U). These bases are typically fixed to a ribose or deoxy ribose at the 1′ position. The use of RNA polynucleotides comprising chemically modified nucleotides have been shown to improve RNA expression, expression rates, half-life and/or expressed protein concentrations. RNA polynucleotides comprising chemically modified nucleotides have also been useful in optimizing protein localization thereby avoiding deleterious bio-responses such as immune responses and/or degradation pathways.

Examples of modified or chemically-modified nucleotides include 5-hydroxycytidines, 5-alkylcytidines, 5-hydroxyalkylcytidines, 5-carboxycytidines, 5-formylcytidines, 5-alkoxycytidines, 5-alkynylcytidines, 5-halocytidines, 2-thiocytidines, N4-alkylcytidines, N4-aminocytidines, N4-acetylcytidines, and N4,N4-dialkylcytidines.

Examples of modified or chemically-modified nucleotides include 5-hydroxycytidine, 5-methylcytidine, 5-hydroxymethylcytidine, 5-carboxycytidine, 5-formylcytidine, 5-methoxycytidine, 5-propynylcytidine, 5-bromocytidine, 5-iodocytidine, 2-thiocytidine; N4-methylcytidine, N4-aminocytidine, N4-acetylcytidine, and N4,N4-dimethylcytidine.

Examples of modified or chemically-modified nucleotides include 5-hydroxyuridines, 5-alkyluridines, 5-hydroxyalkyluridines, 5-carboxyuridines, 5-carboxyalkylesteruridines, 5-formyluridines, 5-alkoxyuridines, 5-alkynyluridines, 5-halouridines, 2-thiouridines, and 6-alkyluridines.

Examples of modified or chemically-modified nucleotides include 5-hydroxyuridine, 5-methyluridine, 5-hydroxymethyluridine, 5-carboxyuridine, 5-carboxymethylesteruridine, 5-formyluridine, 5-methoxyuridine (also referred to herein as “5MeOU”), 5-propynyluridine, 5-bromouridine, 5-fluorouridine, 5-iodouridine, 2-thiouridine, and 6-methyluridine.

Examples of modified or chemically-modified nucleotides include 5-methoxycarbonylmethyl-2-thiouridine, 5-methylaminomethyl-2-thiouridine, 5-carbamoylmethyluridine, 5-carbamoylmethyl-2′-O-methyluridine, 1-methyl-3-(3-amino-3-carboxypropy)pseudouridine, 5-methylaminomethyl-2-selenouridine, 5-carboxymethyluridine, 5-methyldihydrouridine, 5-taurinomethyluridine, 5-taurinomethyl-2-thiouridine, 5-(isopentenylaminomethyl)uridine, 2′-O-methylpseudouridine, 2-thio-2′O-methyluridine, and 3,2′-O-dimethyluridine.

Examples of modified or chemically-modified nucleotides include N6-methyladenosine, 2-aminoadenosine, 3-methyladenosine, 8-azaadenosine, 7-deazaadenosine, 8-oxoadenosine, 8-bromoadenosine, 2-methylthio-N6-methyladenosine, N6-isopentenyladenosine, 2-methylthio-N6-isopentenyladenosine, N6-(cis-hydroxyisopentenyl)adenosine, 2-methylthio-N6-(cis-hydroxyisopentenyl)adenosine, N6-glycinylcarbamoyladenosine, N6-threonylcarbamoyl-adenosine, N6-methyl-N6-threonylcarbamoyl-adenosine, 2-methylthio-N6-threonylcarbamoyl-adenosine, N6,N6-dimethyladenosine, N6-hydroxynorvalylcarbamoyladenosine, 2-methylthio-N6-hydroxynorvalylcarbamoyl-adenosine, N6-acetyl-adenosine, 7-methyl-adenine, 2-methylthio-adenine, 2-methoxy-adenine, alpha-thio-adenosine, 2′-O-methyl-adenosine, N6,2′-O-dimethyl-adenosine, N6,N6,2′-O-trimethyl-adenosine, 1,2′-O-dimethyl-adenosine, 2′-O-ribosyladenosine, 2-amino-N6-methyl-purine, 1-thio-adenosine, 2′-F-ara-adenosine, 2′-F-adenosine, 2′-OH-ara-adenosine, and N6-(19-amino-pentaoxanonadecyl)-adenosine.

Examples of modified or chemically-modified nucleotides include N1-alkylguanosines, N2-alkylguanosines, thienoguanosines, 7-deazaguanosines, 8-oxoguanosines, 8-bromoguanosines, O6-alkylguanosines, xanthosines, inosines, and N1-alkylinosines.

Examples of modified or chemically-modified nucleotides include N1-methylguanosine, N2-methylguanosine, thienoguanosine, 7-deazaguanosine, 8-oxoguanosine, 8-bromoguanosine, O6-methylguanosine, xanthosine, inosine, and N1-methylinosine.

Examples of modified or chemically-modified nucleotides include pseudouridines. Examples of pseudouridines include N1-alkylpseudouridines, N1-cycloalkylpseudouridines, N1-hydroxypseudouridines, N1-hydroxyalkylpseudouridines, N1-phenylpseudouridines, N1-phenylalkylpseudouridines, N1-aminoalkylpseudouridines, N3-alkylpseudouridines, N6-alkylpseudouridines, N6-alkoxypseudouridines, N6-hydroxypseudouridines, N6-hydroxyalkylpseudouridines, N6-morpholinopseudouridines, N6-phenylpseudouridines, and N6-halopseudouridines. Examples of pseudouridines include N1-alkyl-N6-alkylpseudouridines, N1-alkyl-N6-alkoxypseudouridines, N1-alkyl-N6-hydroxypseudouridines, N1-alkyl-N6-hydroxyalkylpseudouridines, N1-alkyl-N6-morpholinopseudouridines, N1-alkyl-N6-phenylpseudouridines, and N1-alkyl-N6-halopseudouridines. In these examples, the alkyl, cycloalkyl, and phenyl substituents may be unsubstituted, or further substituted with alkyl, halo, haloalkyl, amino, or nitro substituents.

Examples of pseudouridines include N1-methylpseudouridine (also referred to herein as “N1MPU”), N1-ethylpseudouridine, N1-propylpseudouridine, N1-cyclopropylpseudouridine, N1-phenylpseudouridine, N1-aminomethylpseudouridine, N3-methylpseudouridine, N1-hydroxypseudouridine, and N1-hydroxymethylpseudouridine.

Examples of nucleic acid monomers include modified and chemically-modified nucleotides, including any such nucleotides known in the art.

Examples of modified and chemically-modified nucleotide monomers include any such nucleotides known in the art, for example, 2′-O-methyl ribonucleotides, 2′-O-methyl purine nucleotides, 2′-deoxy-2′-fluoro ribonucleotides, 2′-deoxy-2′-fluoro pyrimidine nucleotides, 2′-deoxy ribonucleotides, 2′-deoxy purine nucleotides, universal base nucleotides, 5-C-methyl-nucleotides, and inverted deoxyabasic monomer residues.

Examples of modified and chemically-modified nucleotide monomers include 3′-end stabilized nucleotides, 3′-glyceryl nucleotides, 3′-inverted abasic nucleotides, and 3′-inverted thymidine.

Examples of modified and chemically-modified nucleotide monomers include locked nucleic acid nucleotides (LNA), 2′-O,4′-C-methylene-(D-ribofuranosyl) nucleotides, 2′-methoxyethoxy (MOE) nucleotides, 2′-methyl-thio-ethyl, 2′-deoxy-2′-fluoro nucleotides, and 2′-O-methyl nucleotides. In an exemplary embodiment, the modified monomer is a locked nucleic acid nucleotide (LNA).

Examples of modified and chemically-modified nucleotide monomers include 2′,4′-constrained 2′-O-methoxyethyl (cMOE) and 2′-O-Ethyl (cEt) modified DNAs.

Examples of modified and chemically-modified nucleotide monomers include 2′-amino nucleotides, 2′-O-amino nucleotides, 2′-C-allyl nucleotides, and 2′-O-allyl nucleotides.

Examples of modified and chemically-modified nucleotide monomers include N6-methyladenosine nucleotides.

Examples of modified and chemically-modified nucleotide monomers include nucleotide monomers with modified bases 5-(3-amino)propyluridine, 5-(2-mercapto)ethyluridine, 5-bromouridine; 8-bromoguanosine, or 7-deazaadenosine.

Examples of modified and chemically-modified nucleotide monomers include 2′-O-aminopropyl substituted nucleotides.

Examples of modified and chemically-modified nucleotide monomers include replacing the 2′—OH group of a nucleotide with a 2′-R, a 2′-OR, a 2′-halogen, a 2′-SR, or a 2′-amino, where R can be H, alkyl, alkenyl, or alkynyl.

Exemplary base modifications described above can be combined with additional modifications of nucleoside or nucleotide structure, including sugar modifications and linkage modifications. Certain modified or chemically-modified nucleotide monomers may be found in nature.

Preferred nucleotide modifications include N1-methylpseudouridine and 5-methoxyuridine.

### Viral Replication Proteins and Polynucleotides Encoding Them

Provided herein, in some embodiments, are RNA molecules encoding one or more viral replication proteins. As used herein, the term “replication protein” or “viral replication protein” refers to any protein or any protein subunit of a protein complex that functions in replication of a viral genome. Generally, viral replication proteins are non-structural proteins. Viral replication proteins encoded by nucleic acid molecules provided herein can function in the replication of any viral genome. The viral genome can be a single-stranded positive-sense RNA genome, a single-stranded negative-sense RNA genome, a double-stranded RNA genome, a single-stranded positive-sense DNA genome, a single-stranded negative-sense DNA genome, or a double-stranded DNA genome. Viral genomes can include a single nucleic acid molecule or more than one nucleic acid molecule. Nucleic acid molecules provided herein can encode one or more viral replication proteins from any virus or virus family, including animal viruses and plant viruses, for example. Viral replication proteins encoded by polynucleotides included in nucleic acid molecules provided herein can be expressed from self-replicating RNA.

In some embodiments, RNA molecules provided herein can encode one or more togavirus replication proteins. In some aspects, the one or more viral replication proteins encoded by RNA molecules provided herein are alphavirus proteins. In some embodiments, the one or more viral replication proteins encoded by RNA molecules provided herein are rubivirus proteins. RNA molecules provided herein can encode any alphavirus replication protein and any rubivirus replication protein. Exemplary replication proteins from alphaviruses include proteins from Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O'Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), Buggy Creek Virus (BCRV), and any combination thereof. Exemplary rubivirus replication proteins include proteins from rubella virus.

Viral replication proteins encoded by RNA molecules provided herein can be expressed as one or more polyproteins or as separate or single proteins. Generally, polyproteins are precursor proteins that are cleaved to generate individual or separate proteins. Accordingly, proteins derived from a precursor polyprotein can be expressed from a single open reading frame (ORF). As used herein, the term “ORF” refers to a nucleotide sequence that begins with a start codon, generally ATG, and that ends with a stop codon, such as TAA, TAG, or TGA, for example. It will be appreciated that T is present in DNA, while U is present in RNA. Accordingly, a start codon of ATG in DNA corresponds to AUG in RNA, and the stop codons TAA, TAG, and TGA in DNA correspond to UAA, UAG, and UGA in RNA. It will further be appreciated that for any sequence provided in the present disclosure, T is present in DNA, while U is present in RNA. Accordingly, for any sequence provided herein, T present in DNA is substituted with U for an RNA molecule, and U present in RNA is substituted with T for a DNA molecule.

The protease cleaving a polyprotein can be a viral protease or a cellular protease. In some aspects, the RNA molecules provided herein encodes one or more viral replication proteins comprising an alphavirus nsP1 protein, an alphavirus nsP2 protein, an alphavirus nsP3 protein, an alphavirus nsP4 protein, or any combination thereof. In other aspects, the RNA molecules provided herein encodes one or more viral replication proteins comprising an alphavirus nsP1 protein, an alphavirus nsP2 protein, an alphavirus nsP3 protein, or any combination thereof, and an alphavirus nsP4 protein. In some aspects, the polyprotein is a VEEV polyprotein. In other aspects, the alphavirus nsP1, nsP2, nsP3, and nsP4 proteins are VEEV proteins.

In one aspect, the RNA molecules provided herein lack a stop codon between sequences encoding an nsP3 protein and an nsP4 protein. Accordingly, in some aspects, polynucleotides of RNA molecules provided herein encode a P1234 polyprotein comprising nsP1, nsP2, nsP3, and nsP4. The RNA molecules provided herein can also include a stop codon between sequences encoding an nsP3 and an nsP4 protein. Accordingly, in some aspects, polynucleotides of nucleic acid molecules provided herein encode a P123 polyprotein comprising nsP1, nsP2, and nsP3 and a P1234 polyprotein comprising nsP1, nsP2, nsP3, and nsP4 as a result of stop codon readthrough, for example. In one aspect, nsP2 and nsP3 proteins include mutations. Exemplary mutations include G1309R and S1583G mutations of VEEV proteins. In another aspect, the nsP1, nsP2, and nsP4 proteins are VEEV proteins, and the nsP3 protein is a chikungunya virus (CHIKV) nsP3 protein.

In some embodiments, the RNA molecules provided herein encode one or more viral replication proteins comprising a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, or 100% identity to the sequence of SEQ ID NO:13. In some embodiments, the one or more viral replication proteins comprises the sequence of SEQ ID NO:13.

### 5′ Untranslated Region (5′ UTR)

Nucleic acid molecules provided herein can further comprise untranslated regions (UTRs). Untranslated regions, including 5′ UTRs and 3′ UTRs, for example, can affect RNA stability and/or efficiency of RNA translation, such as translation of cellular and viral mRNAs, for example. 5′ UTRs and 3′ UTRs can also affect stability and translation of viral genomic RNAs and self-replicating RNAs, including virally derived self-replicating RNAs or replicons. Exemplary viral genomic RNAs whose stability and/or efficiency of translation can be affected by 5′ UTRs and 3′ UTRs include the genome nucleic acid of positive-sense RNA viruses. Both genome nucleic acid of positive-sense RNA viruses and self-replicating RNAs, including virally derived self-replicating RNAs or replicons, can be translated upon infection or introduction into a cell.

In some aspects, nucleic acid molecules provided herein further include a 5′ untranslated region (5′ UTR). Any 5′ UTR sequence can be included in nucleic acid molecules provided herein. In some embodiments, nucleic acid molecules provided herein include a viral 5′ UTR. In one aspect, nucleic acid molecules provided herein include a non-viral 5′ UTR. Any non-viral 5′ UTR can be included in nucleic acid molecules provided herein, such as 5′ UTRs of transcripts expressed in any cell or organ, including muscle, skin, subcutaneous tissue, liver, spleen, lymph nodes, antigen-presenting cells, and others. In another aspect, nucleic acid molecules provided herein include a 5′ UTR comprising viral and non-viral sequences. Accordingly, a 5′ UTR included in nucleic acid molecules provided herein can comprise a combination of viral and non-viral 5′ UTR sequences. In some aspects, the 5′ UTR included in nucleic acid molecules provided herein is located upstream of or 5′ of a polynucleotide that encodes one or more viral replication proteins. In some aspects, the 5′ UTR is located 5′ of or upstream of a polynucleotide of nucleic acid molecules provided herein that encodes one or more viral replication proteins, which is in turn located 5′ of or upstream of one or more further polynucleotides of nucleic acid molecules provided herein (e.g., one or more of a polynucleotide encoding an NA or an HA polypeptide).

In one aspect, the 5′ UTR of nucleic acid molecules provided herein comprises an alphavirus 5′ UTR. A 5′ UTR from any alphavirus can be included in nucleic acid molecules provided herein, including 5′ UTR sequences from Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O'Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), or Buggy Creek Virus (BCRV). In another aspect, the 5′ UTR comprises a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to the sequence of SEQ ID NO:14, for example. In some embodiments, the 5′ UTR comprises the sequence of SEQ ID NO: 14.

In some embodiments, the 5′ UTR comprises a sequence selected from the 5′ UTRs of human IL-6, alanine aminotransferase 1, human apolipoprotein E, human fibrinogen alpha chain, human transthyretin, human haptoglobin, human alpha-1-antichymotrypsin, human antithrombin, human alpha-1-antitrypsin, human albumin, human beta globin, human complement C3, human complement C5, SynK (thylakoid potassium channel protein derived from the cyanobacteria, Synechocystis sp.), mouse beta globin, mouse albumin, and a tobacco etch virus, or fragments of any of the foregoing. Preferably, the 5′ UTR is derived from a tobacco etch virus (TEV). In one aspect, the 5′ UTR includes a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to sequence of SEQ ID NO:17. In another aspect, the 5′ UTR includes the sequence of SEQ ID NO:17 or SEQ ID NO:14.

An mRNA or any other RNA described herein can comprise any 5′ UTR sequence provided herein. For example, an RNA described herein can comprise a 5′ UTR sequence that is derived from a gene expressed by Arabidopsis thaliana. In some aspects, the 5′ UTR sequence of a gene expressed by Arabidopsis thaliana is ATIG58420. Examples of 5 UTRs and 3′ UTRs are described in US20190002906A1, the contents of which are herein incorporated by reference. Exemplary 5′ UTR sequences include sequences of SEQ ID NOs: 17 and 22-50, as shown in Table 1.

TABLE 1
Exemplary 5′ UTR Sequences

| Name | Sequence | Seq ID No.: |
|---|---|---|
| TEV | UCAACACAACAUAUACAAAACAAACGAAUCUCAAGC | 17 |
| | AAUCAAGCAUUCUACUUCUAUUGCAGCAAUUUAAAU | |
| | CAUUUCUUUUAAAGCAAAAGCAAUUUUCUGAAAAUU | |
| | UUCACCAUUUACGAACGAUAG | |
| AT1G58420 | AUUAUUACAUCAAAACAAAAAGCCGCCA | 22 |
| ARC5-2 | CUUAAGGGGGCGCUGCCUACGGAGGUGGCAGCCAUC | 23 |
| | UCCUUCUCGGCAUCAAGCUUACCAUGGUGCCCCAGGC | |
| | CCUGCUCUUGGUCCCGCUGCUGGUGUUCCCCCUCUGC | |
| | UUCGGCAAGUUCCCCAUCUACACCAUCCCCGACAAGC | |
| | UGGGGCCGUGGAGCCCCAUCGACAUCCACCACCUGUC | |
| | CUGCCCCAACAACCUCGUGGUCGAGGACGAGGGCUGC | |
| | ACCAACCUGAGCGGGUUCUCCUAC | |
| HCV | UGAGUGUCGUACAGCCUCCAGGCCCCCCCCUCCCGGG | 24 |
| | AGAGCCAUAGUGGUCUGCGGAACCGGUGAGUACACC | |
| | GGAAUUGCCGGGAAGACUGGGUCCUUUCUUGGAUAA | |
| | ACCCACUCUAUGCCCGGCCAUUUGGGCGUGCCCCCGC | |
| | AAGACUGCUAGCCGAGUAGUGUUGGGUUGCG | |
| HUMAN | AAUUAUUGGUUAAAGAAGUAUAUUAGUGCUAAUUUC | 25 |
| ALBUMIN | CCUCCGUUUGUCCUAGCUUUUCUCUUCUGUCAACCCC | |
| | ACACGCCUUUGGCACA | |
| EMCV | CUCCCUCCCCCCCCCCUAACGUUACUGGCCGAAGCCG | 26 |
| | CUUGGAAUAAGGCCGGUGUGCGUUUGUCUAUAUGUU | |
| | AUUUUCCACCAUAUUGCCGUCUUUUGGCAAUGUGAG | |
| | GGCCCGGAAACCUGGCCCUGUCUUCUUGACGAGCAU | |
| | UCCUAGGGGUCUUUCCCCUCUCGCCAAAGGAAUGCA | |
| | AGGUCUGUUGAAUGUCGUGAAGGAAGCAGUUCCUCU | |
| | GGAAGCUUCUUGAAGACAAACAACGUCUGUAGCGAC | |
| | CCUUUGCAGGCAGCGGAACCCCCCACCUGGCGACAGG | |
| | UGCCUCUGCGGCCAAAAGCCACGUGUAUAAGAUACA | |
| | CCUGCAAAGGCGGCACAACCCCAGUGCCACGUUGUGA | |
| | GUUGGAUAGUUGUGGAAAGAGUCAAAUGGCUCUCCU | |
| | CAAGCGUAUUCAACAAGGGGCUGAAGGAUGCCCAGA | |
| | AGGUACCCCAUUGUAUGGGAUCUGAUCUGGGGCCUC | |
| | GGUGCACAUGCUUUACGUGUGUUUAGUCGAGGUUAA | |
| | AAAACGUCUAGGCCCCCCGAACCACGGGGACGUGGU | |
| | UUUCCUUUGAAAAACACGAUGAUAAU | |
| AT1G67090 | CACAAAGAGUAAAGAAGAACA | 27 |
| AT1G35720 | AACACUAAAAGUAGAAGAAAA | 28 |
| AT5G45900 | CUCAGAAAGAUAAGAUCAGCC | 29 |
| AT5G61250 | AACCAAUCGAAAGAAACCAAA | 30 |
| AT5G46430 | CUCUAAUCACCAGGAGUAAAA | 31 |
| AT5G47110 | GAGAGAGAUCUUAACAAAAAA | 32 |
| AT1G03110 | UGUGUAACAACAACAACAACA | 33 |
| AT3G12380 | CCGCAGUAGGAAGAGAAAGCC | 34 |
| AT5G45910 | AAAAAAAAAAGAAAUCAUAAA | 35 |
| AT1G07260 | GAGAGAAGAAAGAAGAAGACG | 36 |
| AT3G55500 | CAAUUAAAAAUACUUACCAAA | 37 |
| AT3G46230 | GCAAACAGAGUAAGCGAAACG | 38 |
| AT2G36170 | GCGAAGAAGACGAACGCAAAG | 39 |
| AT1G10660 | UUAGGACUGUAUUGACUGGCC | 40 |
| AT4G14340 | AUCAUCGGAAUUCGGAAAAAG | 41 |
| AT1G49310 | AAAACAAAAGUUAAAGCAGAC | 42 |
| AT4G14360 | UUUAUCUCAAAUAAGAAGGCA | 43 |
| AT1G28520 | GGUGGGGAGGUGAGAUUUCUU | 44 |
| AT1G20160 | UGAUUAGGAAACUACAAAGCC | 45 |
| AT5G37370 | CAUUUUUCAAUUUCAUAAAAC | 46 |
| AT4G11320 | UUACUUUUAAGCCCAACAAAA | 47 |
| AT5G40850 | GGCGUGUGUGUGUGUUGUUGA | 48 |
| AT1G06150 | GUGGUGAAGGGGAAGGUUUAG | 49 |
| AT2G26080 | UUGUUUUUUUUUGGUUUGGUU | 50 |

Additional exemplary 5′ UTR sequences of SEQ ID NOs: 65-111 are shown in Table 2.

TABLE 2
Exemplary 5′ UTR Sequences

| SEQ ID | | |
|---|---|---|
| NO. | SEQUENCE | SOURCE/NAME |
| 65 | AACUUAAAAAAAAAAAUCAAA | SYNECHOCYSTIS sp. PCC6803 |
| | | POTASSIUM CHANNEL |
| | | (SynK) |
| 66 | UCAAGCUUUUGGACCCUCGUACAGAAGCUA | SYNTHETIC SEQUENCE |
| | AUACGACUCACUAUAGGGAAAUAAGAGAGA | |
| | AAAGAAGAGUAAGAAGAAAUAUAAGAGCCA | |
| | CC | |
| 67 | CACAUUUGCUUCUGACAUAGUUGUGUUGAC | MOUSE BETA GLOBIN |
| | UCACAACCCCAGAAACAGACAUC | |
| 68 | ACAUUUGCUUCUGACACAACUGUGUUCACU | HUMAN BETA GLOBIN |
| | AGCAACCUCAAACAGACACC | |
| 69 | UGCACACAGAUCACCUUUCCUAUCAACCCC | MOUSE ALBUMIN |
| | ACUAGCCUCUGGCAAA | |
| 70 | CAUAAACCCUGGCGCGCUCGCGGGCCGGCA | HUMAN ALPHA GLOBIN |
| | CUCUUCUGGUCCCCACAGACUCAGAGAGAA | |
| | CCCACC | |
| 71 | AUAAAAAGACCAGCAGAUGCCCCACAGCAC | HUMAN HAPTOGLOBIN |
| | UGCUCUUCCAGAGGCAAGACCAACCAAG | |
| 72 | AGACAAGGUUCAUAUUUGUAUGGGUUACUU | HUMAN TRANSTHYRETIN |
| | AUUCUCUCUUUGUUGACUAAGUCAAUAAUC | |
| | AGAAUCAGCAGGUUUGCAGUCAGAUUGGCA | |
| | GGGAUAAGCAGCCUAGCUCAGGAGAAGUGA | |
| | GUAUAAAAGCCCCAGGCUGGGAGCAGCCAU | |
| | CACAGAAGUCCACUCAUUCUUGGCAGG | |
| 73 | AGAUAAAAAGCCAGCUCCAGCAGGCGCUGC | HUMAN COMPLEMENT C3 |
| | UCACUCCUCCCCAUCCUCUCCCUCUGUCCCU | |
| | CUGUCCCUCUGACCCUGCACUGUCCCAGCAC | |
| | C | |
| 74 | UAUAUCCGUGGUUUCCUGCUACCUCCAACC | HUMAN COMPLEMENT C5 |
| 75 | GGCACCACCACUGACCUGGGACAGUGAAUC | HUMAN ALPHA-1- |
| | GACA | ANTITRYPSIN |
| 76 | AUUCAUGAAAAUCCACUACUCCAGACAGAC | HUMAN ALPHA-1- |
| | GGCUUUGGAAUCCACCAGCUACAUCCAGCU | ANTICHYMOTRYPSIN |
| | CCCUGAGGCAGAGUUGAGA | |
| 77 | AAUAUUAGAGUCUCAACCCCCAAUAAAUAU | HUMAN INTERLEUKIN 6 |
| | AGGACUGGAGAUGUCUGAGGCUCAUUCUGC | |
| | CCUCGAGCCCACCGGGAACGAAAGAGAAGC | |
| | UCUAUCUCCCCUCCAGGAGCCCAGCU | |
| 78 | AGGAUGGGAACUAGGAGUGGCAGCAAUCCU | HUMAN FIBRINOGEN |
| | UUCUUUCAGCUGGAGUGCUCCUCAGGAGCC | ALPHA CHAIN |
| | AGCCCCACCCUUAGAAAAG | |
| 79 | AGGGGGAGCCCUAUAAUUGGACAAGUCUGG | HUMAN APOLIPOPROTEIN E |
| | GAUCCUUGAGUCCUACUCAGCCCCAGCGGA | |
| | GGUGAAGGACGUCCUUCCCCAGGAGCCGAC | |
| | UGGCCAAUCACAGGCAGGAAG | |
| 80 | AGACGGGUGGGGCGGGGCCCAACUGUCCCC | ALANINE |
| | AGCUCCUUCAGCCCUUUCUGUCCCUCCCAG | AMINOTRANSFERASE 1 |
| | UGAGGCCAGCUGCGGUGAAGAGGGUGCUCU | |
| | CUUGCCUGGAGUUCCCUCUGCUACGGCUGC | |
| | CCCCUCCCAGCCCUGGCCCACUAAGCCAGAC | |
| | CCAGCUGUCGCCAUUCCCACUUCUGGUCCU | |
| | GCCACCUCCUGAGCUGCCUUCCCGCCUGGUC | |
| | UGGGUAGAGUC | |
| 81 | CAGAUCGCCUGGAGACGCCAUCCACGCUGU | HHV |
| | UUUGACCUCCAUAGAAGACACCGGGACCGA | |
| | UCCAGCCUCCGCGGCCGGGAACGGUGCAUU | |
| | GGAACGCGGAUUCCCCGUGCCAAGAGUGAC | |
| | UCACCGUCCUUGACACG | |
| 82 | GGGAGAAAGCUUACCAUGGUGCCCCAGGCC | ARC5-1 |
| | CUGCUCUUGGUCCCGCUGCUGGUUUCCCCC | |
| | UCUGCUUCGGCAAGUUCCCCAUCUACACCA | |
| | UCCCCGACAAGCUGGGGCCGUGGAGCCCCA | |
| | UCGACAUCCACCACCUGUCCUGCCCCAACAA | |
| | CCUCGUGGUCGAGGACGAGGGCUGCACCAA | |
| | CCUGAGCGGGUUCUCCUAC | |
| 83 | GGGGCGCUGCCUACGGAGGUGGCAGCCAUC | ARC5-2 |
| | UCCUUCUCGGCAUCAAGCUUACCAUGGUGC | |
| | CCCAGGCCCUGCUCUUGGUCCCGCUGCUGG | |
| | UGUUCCCCCUCUGCUUCGGCAAGUUCCCCA | |
| | UCUACACCAUCCCCGACAAGCUGGGGCCGU | |
| | GGAGCCCCAUCGACAUCCACCACCUGUCCU | |
| | GCCCCAACAACCUCGUGGUCGAGGACGAGG | |
| | GCUGCACCAACCUGAGCGGGUUCUCCUAC | |
| 84 | GAAUAAAUGUAUAGGGGGAAAGGCAGGAGC | Mouse GROWTH HORMONE |
| | CUUGGGGUCGAGGAAAACAGGUAGGGUAUA | |
| | AAAAGGGCACGCAAGGGACCAAGUCCAGCA | |
| | UCCUAGAGUCCAGAUUCCAAACUGCUCAGA | |
| | GUCCUGUGGACAGAUCACUGCUUGGCA | |
| 85 | GACACUUCUGAUUCUGACAGACUCAGGAAG | MOUSE HEMOGLOBIN |
| | AAACC | ALPHA |
| 86 | UGCAAACACAGAAAUGGAGGAGGAGGGGAA | MOUSE HAPTOGLOBIN |
| | GGAGGAGGAGGAGGAGAAGGAGGAGGAGG | |
| | UGGUGGUGGUGGUGGUGGGAUAAAACCCCU | |
| | GAGGCAUAAAGGGCUCGGCCGGAGUCAGCA | |
| | CAGCCCAGCCCUUCCAGAGAGAGGCAAGAG | |
| | AGGUCCACG | |
| 87 | CUAAUCUCCCUAGGCAAGGUUCAUAUUUGU | MOUSE TRANSTHYRETIN |
| | GUAGGUUACUUAUUCUCCUUUUGUUGACUA | |
| | AGUCAAUAAUCAGAAUCAGCAGGUUUGGAG | |
| | UCAGCUUGGCAGGGAUCAGCAGCCUGGGUU | |
| | GGAAGGAGGGGGUAUAAAAGCCCCUUCACC | |
| | AGGAGAAGCCGUCACACAGAUCCACAAGCU | |
| | CCUGACAGG | |
| 88 | AUAGGUAAUUUUAGAAAUAGAUCUGAUUU | MOUSE ANTITHROMBIN |
| | GUAUCUGAGACAUUUUAGUGAAGUGGUGAG | |
| | AUAUAAGACAUAAUCAGAAGACAUAUCUAC | |
| | CUGAAGACUUUAAGGGGAGAGCUCCCUCCC | |
| | CCACCUGGCCUCUGGACCUCUCAGAUUUAG | |
| | GGGAAAGAACCAGUUUUCGGAGUGAUCGUC | |
| | UCAGUCAGCACCAUCUCUGUAGGAGCAUCG | |
| | GCC | |
| 89 | AGAGAGGAGAGCCAUAUAAAGAGCCAGCGG | MOUSE COMPLEMENT C3 |
| | CUACAGCCCC | |
| | AGCUCGCCUCUGCCCACCCCUGCCCCUUACC | |
| | CCUUCAUUCCUUCCACCUU UUUCCUUCACU | |
| 90 | UUUAAAAGGAAAGUGGUUACAGGGAGGCCA | MOUSE COMPLEMENT C5 |
| | UGCCCAUGGGUUU | |
| 91 | AGUCCUUAGACUGCACAGCAGAACAGAAGG | MOUSE HEPCIDIN |
| | CAUG | |
| 92 | CCCCCAUAUCCCCCUUGGCUCCCAUUGCUUA | MOUSE ALPHA-1- |
| | AAUACAGACUAGGACAGGGCUCUGUCUCCU | ANTITRYPSIN |
| | CAGCCUCGGUCACCACCCAGCUCUGGGACA | |
| | GCAAGCUGAAA | |
| 93 | AGUCAGUCCUCCUUCGCUUCAGCUCCAGUU | MOUSE FIBRINOGEN |
| | CUCCUCAUGAGCCAUCCCUAAACGCAGACA | ALPHA CHAIN |
| | CC | |
| 94 | UUUCCUCUGCCCUGCUGUGAAGGGGGAGAG | APOLIPOPROTEIN E |
| | AACAACCCGCCUCGUGACAGGGGGCUGGCA | |
| | CAGCCCGCCCUAGCCCUGAGGAGGGGGCGG | |
| | GACAGGGGGAGUCCUAUAAUUGGACCGGUC | |
| | UGGGAUCCGAUCCCCUGCUCAGACCCUGGA | |
| | GGCUAAGGACUUGUUUCGGAAGGAGCUGAC | |
| | UGGCCAAUCACAAUUGCGAAG | |
| 95 | GGCCGGCCACCGGGUUUGGGAGCAGCCCAG | ALANINE |
| | GCUCACCUUAACCGGAGCGGUGCGGACGGU | AMINOTRANSFERASE |
| | CCCGCGGCGACAGGGCUAAUCUCGGCAGGU | |
| | UCGCG | |
| 96 | GUCCUGGACUGACUCCCACAACUCUGCCAG | CYTOCHROME P450, |
| | UCUCCAGCCCCUGCCCUUCAGUGGUACAG | FAMILY 1(CYP1A2) |
| 97 | UUUAAGUCAACACCAGGAACUAGGACACAG | PLASMINOGEN |
| | UUGUCCAGGUGCUGUUGGCCAGUCCCAAC | |
| 98 | AAGGAGCUGGGGAGUGGAGUGUAGGCACUA | MOUSE MAJOR URINARY |
| | UAACCUGAAAGACGUGGUCCUGACAGGAGG | PROTEIN 3 (MUP3) |
| | ACAAUUCUAUUCCCUACCAAA | |
| 99 | ACCAGCCAGAAGCCACAGUCUCAUC | MOUSE FVII |
| 100 | AAACAGAGCAGGCAGGGGCCCUGAUUCACU | HNF-1 ALPHA |
| | GGCCGCUGGGGCCAGGGUUGGGGGCUGGGG | |
| | GUGCCCACAGAGCUUGACUAGUGGGAUUUG | |
| | GGGGGGCAGUGGGUGCAGCGAGCCCGGUCC | |
| | GUUGACUGCCAGCCUGCCGGCAGGUAGACA | |
| | CCGGCCGUGGGUGGGGGAGGCGGCUAGCUC | |
| | AGUGGCCUUGGGCCGCGUGGCCUGGUGGCA | |
| | GCGGAGCC | |
| 101 | GGACUUCAGCAGGACUGCUCGAAACAUCCC | MOUSE ALPHA- |
| | ACUUCCAGCACUGCCUGCGGUGAAGGAACC | FETOPROTEIN |
| | AGCAGCC | |
| 102 | AGGGCCUCGUGGGGGGCGGGAAGGUACUGU | MOUSE FIBRONECTIN |
| | CCCAUAUAAGCCUCUGCUCUUGGGGCUCAA | |
| | CCGCUCGCACCCGCUGCGCUGCACAGGGGG | |
| | AGAAAAGGAGCCCAGGGUGUGAGCCGGACA | |
| | ACUUCUGGUCCUCUCCUUCCAUCUCCUUAC | |
| | CGGCGUCCCCACCUCAGGACUUUUCCCGCA | |
| | GGCUGCGAGGGGACCCACAGUUCGUGGCCA | |
| | CUUGCCUCCUGGGGAGGGCGACUCUCCUCC | |
| | CAUCCACUCAAG | |
| 103 | GGGGGAAAAAAAAACAGCCAAAAUAUGCCA | MOUSE RETINOL BINDING |
| | AAAAGCUUCUCACAACAGCUCCUCAGUAGA | PROTEIN 4, PLASMA (RBP4) |
| | AGCAGGGGCCACUUGGGAAAGCCAGGGCCU | |
| | GGACGCUAAUGUUCCAGGCUACAUCAUAGG | |
| | UCCCUUUUCGCUCAGUGAGGCCACCAUCAC | |
| | CACACCAUGGCCACGUAGGCCUCCAGCCAG | |
| | GGCAACAGGACCUGGAGGCCACCCAAGACU | |
| | GCAGCUGGCUGCCGCUGGGUCCCCGGGCCA | |
| | GCUCUUGGCCCCG | |
| 104 | GAACCGCGGCGAGGAGGGGGGUCGGAGGCC | MOUSE PHOSPHOLIPID |
| | CAGACUUAUAAAGGCUGCUGGACCCGCGCU | TRANSFER PROTEIN (PLTP) |
| | ACCCGCCAGACCCCGCCGCCCGGAUCCCCCG | |
| | CGCUGCCUGUCGCCCCACGUGACCACACUAC | |
| | UAAGCUUGGUCGCC | |
| 105 | AGGGACUCAUCAACCAGGCCUGGCCUCUGA | MOUSE ALANINE- |
| | GUUCAACGCAGAGCUAGCUGGGAAAUGUUC | GLYOXYLATE |
| | CGGAUGUUGGCCAAGGCCAGUGUGACGCUG | AMINOTRANSFERASE |
| | GGCUCCAGAGCGGCAGGUUGGGUCCGGACC | (AGXT) |
| 106 | GCUGCCCCUGUGCUGACUGCUGACAGCUGA | ALDEHYDE |
| | CUGACGCUCGCAGCUAGCAGGUACUUCUGG | DEHYDROGENASE 1 |
| | GUUGCUAGCCCAGAGCCCUGGGCCGGUGAC | FAMILY, MEMBER L1 |
| | CCUGUUUUCCCUACUUCCCGUCUUUGACCU | (ALDHIL1) |
| | UGGGUGCCUUCCAACCUUCUGUUGCC | |
| 107 | GGGUGCUAAAAGAAUCACUAGGGUGGGGAG | FUMARYLACETOACETATE |
| | GCGGUCCCAGUGGGGCGGGUAGGGGUGUGU | HYDROLASE (FAH) |
| | GCCAGGUGGUACCGGGUAUUGGCUGGAGGA | |
| | AGGGCAGCCCGGGGUUCGGGGCGGUCCCUG | |
| | AAUCUAAAGGCCCUCGGCUAGUCUGAUCCU | |
| | UGCCCUAAGCAUAGUCCCGUUAGCCAACCC | |
| | CCUACCCGCCGUGGGCUCUGCUGCCCGGUG | |
| | CUCGUCAGC | |
| 108 | AGGAGGACCUUGGCCAGCGGGCAGAAUGGC | FRUCTOSE |
| | AGUUGGUAGAGGAAGGGAGCAAGGGGGUG | BISPHOSPHATASE 1 (FBP1) |
| | UUUCCUGGGACAGGGGGGCGGAGACCUGGA | |
| | GACUAUAGGCUCCCCCAGGACUCAAGUUCA | |
| | UUGAGUUUCUGCAGACACUGAACGGCUUUC | |
| | AGUCUUCCCGCUGUGACUAUCACCUGUGGG | |
| | CUCCACCUGCCUGCACCUUUAGUCAGCACC | |
| | UUUAGCCAGCACCUGCGCCAGACCCCAGCA | |
| 109 | AGGCGCCGGUCAGG | MOUSE GLYCINE N- |
| | | METHYLTRANSFERASE |
| | | (GNMT) |
| 110 | ACCAUCAACC | MOUSE 4- |
| | | HYDROXYPHENYLPYRUVIC |
| | | ACID DIOXYGENASE (HPD) |
| 111 | UCUGCCCCACCCUGUCCUCUGGAACCUCUGC | HUMAN ANTITHROMBIN |
| | GAGAUUUAGAGGAAAGAACCAGUUUUCAGG | |
| | CGGAUUGCCUCAGAUCACACUAUCUCCACU | |
| | UGCCCAGCCCUGUGGAAGAUUAGCGGCC | |

### 3′ Untranslated Region (3′ UTR)

In some aspects, nucleic acid molecules provided herein further include a 3′ untranslated region (3′ UTR). Any 3′ UTR sequence can be included in nucleic acid molecules provided herein. In one aspect, nucleic acid molecules provided herein include a viral 3′ UTR. In another aspect, nucleic acid molecules provided herein include a non-viral 3′ UTR. Any non-viral 3′ UTR can be included in nucleic acid molecules provided herein, such as 3′ UTRs of transcripts expressed in any cell or organ, including muscle, skin, subcutaneous tissue, liver, spleen, lymph nodes, antigen-presenting cells, and others. In some aspects, nucleic acid molecules provided herein include a 3′ UTR comprising viral and non-viral sequences. Accordingly, a 3′ UTR included in nucleic acid molecules provided herein can comprise a combination of viral and non-viral 3′ UTR sequences. In one aspect, the 3′ UTR is located 3′ of or downstream of a polynucleotide of nucleic acid molecules provided herein that comprises a first transgene encoding a first antigenic protein or a fragment thereof (e.g., an NA or HA polypeptide). In some aspects, the 3′ UTR is located 3′ of or downstream of the polynucleotide of nucleic acid molecules provided herein that comprises a first transgene encoding a first antigenic protein or a fragment thereof, which in turn is located 3′ of or downstream of one or more additional polynucleotides of nucleic acid molecules provided herein (e.g., a polynucleotide encoding another antigenic protein or fragment thereof, and/or a polynucleotide encoding one or more viral replication proteins).

In one aspect, the 3′ UTR of nucleic acid molecules provided herein comprises an alphavirus 3′ UTR. A 3′ UTR from any alphavirus can be included in nucleic acid molecules provided herein, including 3′ UTR sequences from Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O'Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), or Buggy Creek Virus (BCRV). In some embodiments, the 3′ UTR comprises a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to the sequence of SEQ ID NO:15, for example. In yet another aspect, the 3′ UTR further comprises a poly-A sequence. In some embodiments, the 3′ UTR comprises the sequence of SEQ ID NO:15. In some embodiments, the 3′ UTR comprises the sequence of SEQ ID NO:16, for example. In some embodiments, the 3′ UTR comprises a poly-A tail of about 20-300 nucleotides. In some embodiments, the poly-A tail comprises about 20-300 consecutive A nucleotides. In some embodiments, the poly-A tail comprises about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, and any number or range in between, nucleotides. In some embodiments, the nucleotides of the poly-A tail are A nucleotides. In some embodiments, the 3′ UTR includes a poly-C tail.

In some embodiments, the 3′ UTR comprises a sequence selected from the 3′ UTRs of alanine aminotransferase 1, human apolipoprotein E, human fibrinogen alpha chain, human haptoglobin, human antithrombin, human alpha globin, human beta globin, human complement C₃, human growth factor, human hepcidin, MALAT-1, mouse beta globin, mouse albumin, and Xenopus beta globin, or fragments of any of the foregoing. In some embodiments, the 3′ UTR is derived from Xenopus beta globin. Any 3′ UTR provided herein can include a poly-A tail, as detailed further below. In some embodiments, the 3′ UTR includes a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to the sequence of SEQ ID NO:18 or SEQ ID NO:19. In some embodiments, the 3′ UTR includes the sequence of SEQ ID NO:18, or SEQ ID NO:19. A 3′ UTR provided herein can be included in any RNA molecule provided herein, including self-replicating RNA and mRNA molecules. Exemplary 3′ UTR sequences include SEQ ID NOs:51-57, as shown in Table 3.

TABLE 3
3′ UTR sequences

| Name | Sequence | Seq ID No.: |
|---|---|---|
| XBG | CUAGUGACUGACUAGGAUCUGGUUACCACUAAAC | 51 |
| | CAGCCUCAAGAACACCCGAAUGGAGUCUCUAAGC | |
| | UACAUAAUACCAACUUACACUUACAAAAUGUUG | |
| | UCCCCCAAAAUGUAGCCAUUCGUAUCUGCUCCUA | |
| | AUAAAAAGAAAGUUUCUUCACAU | |
| HUMAN | UGCAAGGCUGGCCGGAAGCCCUUGCCUGAAAGCA | 52 |
| HAPTOGLOBIN | AGAUUUCAGCCUGGAAGAGGGCAAAGUGGACGG | |
| | GAGUGGACAGGAGUGGAUGCGAUAAGAUGUGGU | |
| | UUGAAGCUGAUGGGUGCCAGCCCUGCAUUGCUGA | |
| | GUCAAUCAAUAAAGAGCUUUCUUUUGACCCAU | |
| HUMAN | ACGCCGAAGCCUGCAGCCAUGCGACCCCACGCCA | 53 |
| APOLIPOPROT | CCCCGUGCCUCCUGCCUCCGCGCAGCCUGCAGCG | |
| EIN E | GGAGACCCUGUCCCCGCCCCAGCCGUCCUCCUGG | |
| | GGUGGACCCUAGUUUAAUAAAGAUUCACCAAGU | |
| | UUCACGCA | |
| HCV | UAGAGCGGCAAACCCUAGCUACACUCCAUAGCUA | 54 |
| | GUUUCUUUUUUUUUUGUUUUUUUUUUUUUUUUU | |
| | UUUUUUUUUUUUUUUUUUUUUUCCUUUCUUUUC | |
| | CUUCUUUUUUUCCUCUUUUCUUGGUGGCUCCAUC | |
| | UUAGCCCUAGUCACGGCUAGCUGUGAAAGGUCCG | |
| | UGAGCCGCAUGACUGCAGAGAGUGCCGUAACUGG | |
| | UCUCUCUGCAGAUCAUGU | |
| MOUSE | ACACAUCACAACCACAACCUUCUCAGGCUACCCU | 55 |
| ALBUMIN | GAGAAAAAAAGACAUGAAGACUCAGGACUCAUC | |
| | UUUUCUGUUGGUGUAAAAUCAACACCCUAAGGA | |
| | ACACAAAUUUCUUUAAACAUUUGACUUCUUGUC | |
| | UCUGUGCUGCAAUUAAUAAAAAAUGGAAAGAAU | |
| | CUAC | |
| HUMAN | GCUGGAGCCUCGGUAGCCGUUCCUCCUGCCCGCU | 56 |
| ALPHA | GGGCCUCCCAACGGGCCCUCCUCCCCUCCUUGCA | |
| GLOBIN | CCGGCCCUUCCUGGUCUUUGAAUAAAGUCUGAGU | |
| | GGGCAGCA | |
| EMCV | UAGUGCAGUCAC UGGCACAACG CGUUGCCCGG | 57 |
| | UAAGCCAAUC GGGUAUACAC | |
| | GGUCGUCAUACUGCAGACAG GGUUCUUCUA | |
| | CUUUGCAAGA UAGUCUAGAG UAGUAAAAUA | |
| | AAUAGUAUAAG | |

Additional exemplary 3′ UTR sequences of SEQ ID NOs:112-149 are shown in Table 4.

TABLE 4
Exemplary 3′ UTR Sequences

| SEQ | | |
|---|---|---|
| ID | | |
| NO. | SEQUENCE | SOURCE/NAME |
| 112 | ACCCCCUUUCCUGCUCUUGCCUGUGAACAAU | MOUSE BETA GLOBIN |
| | GGUUAAUUGUUCCCAAGAGAGCAUCUGUCAG | |
| | UUGUUGGCAAAAUGAUAAAGACAUUUGAAA | |
| | AUCUGUCUUCUGACAAAUAAAAAGCAUUUAU | |
| | UUCACUGCAAUGAUGUUUU | |
| 113 | GCUCGCUUUCUUGCUGUCCAAUUUCUAUUAA | HUMAN BETA GLOBIN |
| | AGGUUCCUUUGUUCCCUAAGUCCAACUACUA | |
| | AACUGGGGGAUAUUAUGAAGGGCCUUGAGCA | |
| | UCUGGAUUCUGCCUAAUAAAAAACAUUUAUU | |
| | UUCAUUGCAA | |
| 114 | UGGCAUCCCUGUGACCCCUCCCCAGUGCCUC | HUMAN GROWTH FACTOR |
| | UCCUGGCCCUGGAAGUUGCCACUCCAGUGCC | |
| | CACCAGCCUUGUCCUAAUAAAAUUAAGUUGC | |
| | AUCAUUUUGUCUG | |
| 115 | AAUGUUCUUAUUCUUUGCACCUCUUCCUAUU | HUMAN ANTITHROMBIN |
| | UUUGGUUUGUGAACAGAAGUAAAAAUAAAU | |
| | ACAAACUACUUCCAUCUCA | |
| 116 | CCACACCCCCAUUCCCCCACUCCAGAUAAAG | HUMAN COMPLEMENT C3 |
| | CUUCAGUUAUAUCUCACGUGUCUGGAGUUCU | |
| | UUGCCAAGAGGGAGAGGCUGAAAUCCCCAGC | |
| | CGCCUCACCUGCAGCUCAGCUCCAUCCUACU | |
| | UGAAACCUCACCUGUUCCCACCGCAUUUUCU | |
| | CCUGGCGUUCGCCUGCUAGUGUG | |
| 117 | AACCUACCUGCCCUGCCCCCGUCCCCUCCCUU | HUMAN HEPCIDIN |
| | CCUUAUUUAUUCCUGCUGCCCCAGAACAUAG | |
| | GUCUUGGAAUAAAAUGGCUGGUUCUUUUGU | |
| | UUUCCAAA | |
| 118 | ACUAAGUUAAAUAUUUCUGCACAGUGUUCCC | HUMAN FIBRINOGEN |
| | AUGGCCCCUUGCAUUUCCUUCUUAACUCUCU | ALPHA CHAIN |
| | GUUACACGUCAUUGAAACUACACUUUUUUGG | |
| | UCUGUUUUUGUGCUAGACUGUAAGUUCCUUG | |
| | GGGGCAGGGCCUUUGUCUGUCUCAUCUCUGU | |
| | AUUCCCAAAUGCCUAACAGUACAGAGCCAUG | |
| | ACUCAAUAAAUACAUGUUAAAUGGAUGAAU | |
| | GAAUUCCUCUGAAACUCU | |
| 119 | GCACCCCAGCUGGGGCCAGGCUGGGUCGCCC | ALANINE |
| | UGGACUGUGUGCUCAGGAGCCCUGGGAGGCU | AMINOTRANSFERASE 1 |
| | CUGGAGCCCACUGUACUUGCUCUUGAUGCCU | |
| | GGCGGGGUGGGGUGGGGGGGGUGCUGGGCCC | |
| | CUGCCUCUCUGCAGGUCCCUAAUAAAGCUGU | |
| | GUGGCAGUCUGACUCC | |
| 120 | GAUUCGUCAGUAGGGUUGUAAAGGUUUUUC | MALAT |
| | UUUUCCUGAGAAAACAACCUUUUGUUUUCUC | |
| | AGGUUUUGCUUUUUGGCCUUUCCCUAGCUUU | |
| | AAAAAAAAAAAAGCAAAA | |
| 121 | GGACUAGUUAUAAGACUGACUAGCCCGAUGG | ARC3-1 |
| | GCCUCCCAACGGGCCCUCCUCCCCUCCUUGCA | |
| | CCGAGAUUAAU | |
| 122 | GGACUAGUGCAUCACAUUUAAAAGCAUCUCA | ARC3-2 |
| | GCCUACCAUGAGAAUAAGAGAAAGAAAAUGA | |
| | AGAUCAAUAGCUUAUUCAUCUCUUUUUCUUU | |
| | UUCGUUGGUGUAAAGCCAACACCCUGUCUAA | |
| | AAAACAUAAAUUUCUUUAAUCAUUUUGCCUC | |
| | UUUUCUCUGUGCUUCAAUUAAUAAAAAAUGG | |
| | AAAGAACCUAGAUCU | |
| 123 | CCACUCACCAGUGUCUCUGCUGCACUCUCCU | MOUSE GROWTH |
| | GUGCCUCCCUGCCCCCUGGCAACUGCCACCCC | HORMONE |
| | UGCGCUUUGUCCUAAUAAAAUUAAGAUGCAU | |
| | CAUAUCACCCG | |
| 124 | GCUGCCUUCUGCGGGGCUUGCCUUCUGGCCA | MOUSE HEMOGLOBIN |
| | UGCCCUUCUUCUCUCCCUUGCACCUGUACCU | ALPHA |
| | CUUGGUCUUUGAAUAAAGCCUGAGUAGGAAG | |
| | AAAAAAAAAAAA | |
| 125 | UUCAGGGCUCACUAGAAGGCUGCACAUGGCA | MOUSE HAPTOGLOBIN |
| | GGGCAGGCUGGGAGCCAUGGAAGAGGGGGAA | |
| | GUGGAAGGGUUGGGCUAUACUCUGAUGGGU | |
| | UCUAGCCCUGCACUGCUCAGUCAACAAUAAA | |
| | AAAAUGUGCUUUGGACCCAUAAAAAAAAAAA | |
| | AAAAAAAAA | |
| 126 | GAGACUCAGCCCAGGAGGACCAGGAUCUUGC | MOUSE TRANSTHYRETIN |
| | CAAAGCAGUAGCAUCCCAUUUGUACCAAAAC | |
| | AGUGUUCUUGCUCUAUAAACCGUGUUAGCAG | |
| | CUCAGGAAGAUGCCGUGAAGCAUUCUUAUUA | |
| | AACCACCUGCUAUUUCAUUCAAACUGUGUUU | |
| | CUUUUUUAUUUCCUCAUUUUUCUCCCCUGCU | |
| | CCUAAAACCCAAAAUCUUCUAAAGAAUUCUA | |
| | GAAGGUAUGCGAUCAAACUUUUUAAAGAAA | |
| | GAAAAUACUUUUUGACUCAUGGUUUAAAGGC | |
| | AUCCUUUCCAUCUUGGGGAGGUCAUGGGUGC | |
| | UCCUGGCAACUUGCUUGAGGAAGAUAGGUCA | |
| | GAAAGCAGAGUGGACCAACCGUUCAAUGUUU | |
| | UACAAGCAAAACAUACACUAAGCAUGGUCUG | |
| | UAGCUAUUAAAAGCACACAAUCUGAAGGGCU | |
| | GUAGAUGCACAGUAGUGUUUUCCCAGAGCAU | |
| | GUUCAAAAGCCCUGGGUUCAAUCACAAUACU | |
| | GAAAAGUAGGCCAAAAAACAUUCUGAAAAUG | |
| | AAAUAUUUGGGUUUUUUUUUAUAACCUUUA | |
| | GUGACUAAAUAAAGACAAAUCUAAGAGACUA | |
| | AAAAAAAAAAAAAAAAA | |
| 127 | AAUAUUCUUAAUCUUUGCACCUUUUCCUACU | MOUSE ANTITHROMBIN |
| | UUGGUGUUUGUGAAUAGAAGUAAAAAUAAA | |
| | UACGACUGCCACCUCACGAGAAUGGACUUUU | |
| | CCACUUGAAGACGAGAGACUGGAGUACAGAU | |
| | GCUACACCACUUUUGGGCAAGUGAAGGGGGA | |
| | GCAGCCAGCCACGGUGGCACAAACCUAUAUC | |
| | CUGGUGCUUUUGAAGGUAGAAGCAGGGCGGU | |
| | CAGGAGUUAAGGCCAGUUGAGGCUGGGCUGC | |
| | AGAGUGAAAGACCAUGUCUCAAGAUGGUCUU | |
| | UCUCCUCCCCAAAGUAGAAAAGAAAACCAUA | |
| | AAAACAAGAGGUAAAUAUAUUACUAUUUCA | |
| | UCUUAGAGGAUAGCAGGCAUCUUGAAAGGGU | |
| | AGAGGGACCUUAAAUUCUCAUUAUUGCCCCC | |
| | AUACUACAAACUAAAAAACAAACCCGAAUCA | |
| | AUCUCCCAUAAAGACAGAGAUUCAAAUAAGA | |
| | GUAUUAAACGUUUUAUUUCUCAAACCACUCA | |
| | CAUGCAUAAUGUUCUUAUACACAGUGUCAAA | |
| | AUAAAGAGAAAUGCAUUUUUAUACAAAAAA | |
| | AAAAAA | |
| 128 | CUACAGCCCAGCCCUCUAAUAAAGCUUCAGU | MOUSE COMPLEMENT C3 |
| | UGUAUUUCACCCAUC | |
| 129 | AAAGUUCUGCUGCACGAAGAUUCCUCCUGCG | MOUSE COMPLEMENT C5 |
| | GCGGGGGGAUUGCUCCUCCUCUGGCUUGGAA | |
| | ACCUAGCCUAGAAUCAGAUACACUUUCUUUA | |
| | GAGUAAAGCACAAGCUGAUGAGUUACGACUU | |
| | UGUGAAAUGGAUAGCCUUGAGGGGAGGCGA | |
| | AAACAGGUCCCCCAAGGCUAUCAGAUGUCAG | |
| | UGCCAAUAGACUGAAACAAGUCUGUAAAGUU | |
| | AGCAGUCAGGGGUGUUGGUUGGGGCCGGAAG | |
| | AAGAGACCCACUGAAACUGUAGCCCCUUAUC | |
| | AAAACAUAUCCUUGCUUGAAAGAAAAAUACC | |
| | AAGGACAGAAAAUGCCAUAAAAUCUUGACUU | |
| | UGCACUC | |
| 130 | CCUAGAGCCACAUCCUGACCUCUCUACACCC | MOUSE HEPCIDIN |
| | CUGCAGCCCCUCAACCCCAUUAUUUAUUCCU | |
| | GCCCUCCCCACCAAUGACCUUGAAAUAAAGA | |
| | CGAUUUUAUUUUCAAAAAAAAAAAAAAAAA | |
| | A | |
| 131 | CCACCCUAAAAUGUCAUCCUUCCUUCUGAAU | MOUSE ALPHA-1- |
| | UGGGUUCCUUCCAUUAAACACAGGCUGGCCU | ANTITRYPSIN |
| | GGCUCGUGCCUGAUGCUACAGCAAGUCCUUG | |
| | ACUCUGUGGGUUGUGUGUGUGUGUGUGUGU | |
| | GUGUGUGUGUGUGUGUGUGUGUGUGUGUGU | |
| | GUGUGUGUGUGUGUGUCUGUGUGUGUGUGU | |
| | GUCUUUAUGCCCUGAGUUUGUUGUGGACUUG | |
| | AGAUCAUAGUAUGUCUUGAUAUCUCCUCCAG | |
| | CCAUGCAAAUAGGUUGUGGGUAGAGGACUGU | |
| | GGCUGAGACCACAGACUCUGGUCCAAGAACC | |
| | AUCUGCUCUAAAAAAAAUAAAUCUGUCAUCU | |
| | CUGGAAAAUAAAGAGGACAUGCUCAAUGACU | |
| | CAGGGUCCAGC | |
| 132 | CUGAAGGGUUAGAAAGUGGGGGCUCUGUUU | MOUSE FIBRINOGEN |
| | UCUUUGCUCGGUUAUCCGAGAAGAAAGACAA | ALPHA CHAIN |
| | AACGGAAGAUGAAGGUGUCACGGAUCUUGUG | |
| | AACUUUUUAAAACUUUCAAGGUGCUAUUCCA | |
| | UUGUUCUUUGUACUGUAGCUAAAUGUAACUG | |
| | AGAUGAGUUACUGCUUUGAAAAAAUAAAGU | |
| | UUUACAUUUUUUCCACCCUUUAAAAAAAAAA | |
| | AA | |
| 133 | GUAUCCUUCUCCUGUCCUGCAACAACAUCCA | APOLIPOPROTEIN E |
| | UAUCCAGCCAGGUGGCCCUGUCUCAAGCACC | |
| | UCUCUGGCCCUCUGGUGGCCCUUGCUUAAUA | |
| | AAGAUUCUCCGAGCACAUUCUGAGUCUCUGU | |
| | GAGUGAUUCAAAAAAAA | |
| 134 | GGACGCCUCAGGCACCGGAGCCAGACCCUCC | ALANINE |
| | CAAGACCACCCAGGCCUUCCUCAAGGACUCU | AMINOTRANSFERASE |
| | GCCUCAGACCUCAGACAGGCCACCAACGCUG | |
| | UUCAUCUUCAUUUCCCCAAGGAGACUUCUUU | |
| | CUUUGUGCCUUGAUGUUUGAGAGUUCUUCGA | |
| | GCAAACAGUGGUUUUGCAAUGUCUCACAGGC | |
| | CCUGUUUUUGUUUUUGUUUUUGUUUUGUUU | |
| | UGUUUUGUUCUUUUUUUAAAUGCAACCAAAG | |
| | UAGAGUCAACCUGCUCGGCAGAUGUACUUGG | |
| | AUUCUCUGAAUCGCUAUUCUGUUUGGAGAGU | |
| | UCCUUUGGGUCUUAAGCAGCCAGAGUACAUG | |
| | GAAAUGAGAUUAUGUCAGAUCUGGAGAAAC | |
| | AAGCAGGUGUUGGGAAAUAUGUGACUUGAC | |
| | AUGAUAAGGGCUGGGAAUCCAGAAAUCAAUA | |
| | GUGAGAUCCAUGAAAUCAAACCCUGACCAGU | |
| | GUGAAAAUGUAGCCUUUUGGACAGUAAGCCU | |
| | GCAAGUCUAGUGAGAACUCAGAGAAAGCUGA | |
| | CCAUUCUGGUCUGAAGAUAGGCAGCGCAUCA | |
| | CAGGCAAGAAUAUCGAAGUCAGUAGUAGGAC | |
| | AGGGGUCACAUCAGAUACCAGCUCAAAUUGC | |
| | ACUAGCUAUCUAGAACAGUUUUCUCCAGGUU | |
| | UGCCUGAGCCUUGAUGCAUACCAUCGCCCUC | |
| | UGCUGGUCGCAGCAGAGAUAAGCAAGGGCUG | |
| | AAAAUGGAGGCAAUCCUUUCCCAAGGCCCUG | |
| | AAAGUUGUUUUUCAUGGUUUCAAACUGAAU | |
| | UUGGCUCAUUUGUAACUAACUGAUCACGGUG | |
| | CCUGGUUACACUGGCUGCCAAGAAGGAGCGC | |
| | AUGCAAUCUGAUUCAGUGCUCUCUUCACAUC | |
| | AGUUUCCUGCCUCCCUCCCUCAUCUGCGGAC | |
| | AGCAUCCUAUCUCAUCAGGCUUCCCUGUGUG | |
| | UCACAAAGUAGCAGCCACCAAGCAAAUAUAU | |
| | UCCUUGAAUUAGCACACCUGGGUGGGCCAUG | |
| | UGCGCACCAAGGAAACAGGUGCUAUAGGGAG | |
| | CGCCAGGCCAGGCUUGUCUCUUAACUGUCUC | |
| | GUUCUUCAGUGAGAGUGGGAAAGCUGUCCGG | |
| | AGCUCCCGCGCAGGAGCCUGGGUACCCACGC | |
| | AGCGAGUCAAGGGAGUUUUCGGAGCCAGAGA | |
| | GAGAAAGAUGUGAAGGCUGUGGAGUAAGGC | |
| | UGAAACCAGCCUCCUGCCCUAUAGUCCCACA | |
| | CUGCAGGGGGUGCGACUUUAAAACAGAACUU | |
| | CAAGUUGUUAACACUCACAAGCAUUGCAUUA | |
| | CUGUGAAGGAAGUAGCCGCAUCCAUAACAGG | |
| | AUGUGAUGGUCUACAGCUUUUCCUUUAAAAG | |
| | CUGAAAAGGUACCAUGUGUGCUCGCUAGGCA | |
| | UAUAAUCCAGAUAUGCUCCAGAGUUCUGAGA | |
| | UUCUUCCAUGAAAGGUUAACUAGAAGCUAGA | |
| | AUAUUUUUUUAUAUUUUUGUAACAAUUGGC | |
| | UUUUUUCAUGGGGGGAGGGGAGUAGAGGGU | |
| | UAGUAUUUAUAGUCCUAACAAGUCCAAAAAU | |
| | UUUUAUAAGUGUCUUCAGAUUAUAAAUAACC | |
| | CUCCAAAUUUUGCAAUGUUUACAUGUUUUUU | |
| | UUUUAAGAUGACAAAUAUGCUUGAUUUGCU | |
| | UUUUAAAUAAAAGUUUAGCUGUUCUAAGAG | |
| | AUUAACUUCAAGUAGGAUGGCUGGUUAUGA | |
| | UAGUUUGGAUUUUCUACAGGUUCUGUUGCCA | |
| | UGCCUUUUGGGUUUCAGCAUCACUCGAGUCG | |
| | CAGCAUGUGGGUGGGGCUGUGGAAACCUGGC | |
| | CAGGCUGGACCUGGUCAGCCACACCUCAGAG | |
| | ACAUUGUUUCCAUUUGGAUGUGAGCAGGCGC | |
| | AGGCCUGCAUGCUCUUUCCUACUUAGCAUCA | |
| | UCAGUUCUUCCGCCUCCUUAGCAUGGUUCUU | |
| | UGUAACAGCCAUGCUGGGAAGCUCUGAACAA | |
| | UAAAAUACUUCCAGAGUGGU | |
| 135 | AGAUUGUCGAGGCAUCGGUGGGGCCGUCACC | CYTOCHROME P450, |
| | CUUGUUUCUUUUCCUUUUUUAAAAAAAAAAA | FAMILY 1(CYP1A2) |
| | AAAAACAGCUUUUUUUUUUUUGAGAGAUAC | |
| | AAUUCUUUCCCCAUUUAAUUCAUCUCCAAGC | |
| | AAUUUUACAAUAGUGUCUAUCAUGUUCACCC | |
| | CAUAACCCAUACUCAUUAGGACUUAUGAUUU | |
| | AAGAUUCCUCCUACCCUGUCUUGCUUGCCGC | |
| | ACCUCAUGCUAAUCUAGUUUUUGACUCAAUA | |
| | GAUUUGCCUACUCUGGCUGUCUCAUAUAAAU | |
| | CGAAUGAAUUAUG | |
| 136 | CUAGGUGGAAGGCCGAGCAAAACCUCUGCUU | PLASMINOGEN |
| | ACUAAAGCUUACUGAAUAUGGGGAGAGGGCU | |
| | UAGGGUGUUUGGAAAAACUGACAGUAAUCA | |
| | AACUGGGACACUACACUGAACCACAGCUUCC | |
| | UGUCGCCCCUCAGCCCCUCCCCUUUUUUUGU | |
| | AUUAUUGUGGGUAAAAUUUUCCUGUCUGUG | |
| | GACUUCUGGAUUUUGUGACAAUAGACCAUCA | |
| | CUGCUGUGACCUUUGUUGAAAAUAAACUCGA | |
| | UACUUACUUUG | |
| 137 | AGAAUGGCCUGAGCCUCCAGUGUUGAGUGGA | MOUSE MAJOR URINARY |
| | GACUUUUCACCAGGACUCCAGCAUCAUCCCU | PROTEIN 3 (MUP3) |
| | UCCUAUCCAUACAGACUCCCAUGCCAAGGUC | |
| | UGUGAUCUGCUCUCCACCUGUCUCACAGAGA | |
| | AGUGCAAUCCCGUUCUCUCCAGCAUGUUACC | |
| | UAGGAUAACUCAUCAAGAAUCAAAGACUUUC | |
| | UUUAAAUUUCUCUUUGCCAACACAUGGAAAU | |
| | UCUCCAUUGAUUUCUUUCCUGUCCUGUUCAA | |
| | UAAAUGAUUACACUUGCACUUAAAAAAAAAA | |
| | AAA | |
| 138 | CUCCUUGGAUAGCCCAACCCGUCCCAAGAAG | MOUSE FVII |
| | GAAGCUACGGCCUGUGAAGCUGUUCUAUGGA | |
| | CUUUCCUGCUAUUCUUGUGUAAGGGAAGAGA | |
| | AUGAGAUAAAGAGAGAGUGAAGAAAGCAGA | |
| | GGGGGAGGUAAAUGAGAGAGGCUGGGAAAG | |
| | GGGAAACAGAAAGCAGGGCCGGGGGAAGAGU | |
| | CUAAGUUAGAGACUCACAAAGAAACUCAAGA | |
| | GGGGCUGGGCAGUGCAGUCACAGUCAGGCAG | |
| | CUGAGGGGCAGGGUGUCCCUGAGGGAGGCGA | |
| | GGCUCAGGCCUUGCUCCCGUCUCCCCGUAGC | |
| | UGCCUCCUGUCUGCAUGCAUUCGGUCUGCAG | |
| | UACUACACAGUAGGUAUGCACAUGAGCACGU | |
| | AGGACACGUGAAUGUGCCGCAUGCAUGUGCG | |
| | UGCCUGUGUGUCCAUCAUUGGCACUGUUGCU | |
| | CACUUGUGCUUCCUGUGAGCACCCUGUCUUG | |
| | GUUUCAAUUAAAUGAGAAACAUGGUCAAAA | |
| | AAAAAAAAAAAAAAAA | |
| 139 | CCGUGGUGACUGCCUCCCAGGAGCUGGGUCC | HNF-1ALPHA |
| | CCAGGGCCUGCACUGCCUGCAUAGGGGGUGA | |
| | GGAGGGCCGCAGCCACACUGCCUGGAGGAUA | |
| | UCUGAGCCUGCCAUGCCACCUGACACAGGCU | |
| | GCUGGCCUUCCCAGAAGUCUACGCAUUCAUU | |
| | GACACUGCUGCUCCUCCAUCAUCAGGAAGGG | |
| | AUGGCUCUGAGGUGUCUCAGCCUGACAAGCG | |
| | AGCCUCGAGGAGCUGGAGGACGGCCCAAUCU | |
| | GGGCAGUAUUGUGGACCACCAUCCCUGCUGU | |
| | UUAGAAUAGGAAAUUUAAUGCUUGGGACAG | |
| | GAGUGGGGAAGCUCGUGGUGCCCGCACCCCC | |
| | CCAGUCAGAGCCUGCAGGCCUUCAAGGAUCU | |
| | GUGCUGAGCUCUGAGGCCCUAGAUCAACACA | |
| | GCUGCCUGCUGCCUCCUGCACCUCCCCAGGC | |
| | CAUUCCACCCUGCACCAGAGACCCACGUGCC | |
| | UGUUUGAGGAUUACCCUCCCCACCACGGGGA | |
| | UUUCCUACCCAGCUGUUCUGCUAGGCUCGGG | |
| | AGCUGAGGGGAAGCCACUCGGGGCUCUCCUA | |
| | GGCUUUCCCCUACCAAGCCAUCCCUUCUCCC | |
| | AGCCCCAGGACUGCACUUGCAGGCCAUCUGU | |
| | UCCCUUGGAUGUGUCUUCUGAUGCCAGCCUG | |
| | GCAACUUGCAUCCACUAGAAAGGCCAUUUCA | |
| | GGGCUCGGGUUGUCAUCCCUGUUCCUUAGGA | |
| | CCUGCAACUCAUGCCAAGACCACACCAUGGA | |
| | CAAUCCACUCCUCUGCCUGUAGGCCCCUGAC | |
| | AACUUCCUUCCUGCUAUGAGGGAGACCUGCA | |
| | GAACUCAGAAGUCAAGGCCUGGGCAGUGUCU | |
| | AGUGGAGAGGGUACCAAGACCAGCAGAGAGA | |
| | AGCCACCUAAGUGGCCUGGGGGCUAGCAGCC | |
| | AUUCUGAGAAAUCCUGGGUCCCGAGCAGCCC | |
| | AGGGAAACACAGCACACAUGACUGUCUCCUC | |
| | GGGCCUACUGCAGGGAACCUGGCCUUCAGCC | |
| | AGCUCCUUUGUCAUCCUGGACUGUAGCCUAC | |
| | GGCCAACCAUAAGUGAGCCUGUAUGUUUAUU | |
| | UAACUUUUAGUAAAGUCAGUAAAAAGCAAA | |
| | AAAAAAAAAAAAAAA | |
| 140 | ACAUCUCCAGAAGGAAGAGUGGACAAAAAAA | MOUSE ALPHA- |
| | UGUGUUGACUCUUUGGUGUGAGCCUUUUGGC | FETOPROTEIN |
| | UUAACUGUAACUGCUAGUACUUUAACCACAU | |
| | GGUGAAGAUGUCCAUGUGAGAUUUCUAUACC | |
| | UUAGGAAUAAAAACUUUUCAACUAUUUCUCU | |
| | UCUCCUAGUCUGCUUUUUUUUUAUUAAAAAA | |
| | UACUUUUUUCCAUUU | |
| 141 | UCUUUCCAGCCCCACCCUACAAGUGUCUCUC | MOUSE FIBRONECTIN |
| | UACCAAGGUCAAUCCACACCCCAGUGAUGUU | |
| | AGCAGACCCUCCAUCUUUGAGUGGUCCUUUC | |
| | ACCCUUAAGCCUUUUGCUCUGGAGCCAUGUU | |
| | CUCAGCUUCAGCACAAUUUACAGCUUCUCCA | |
| | AGCAUCGCCCCGUGGGAUGUUUUGAGACUUC | |
| | UCUCCUCAAUGGUGACAGUUGGUCACCCUGU | |
| | UCUGCUUCAGGGUUUCAGUACUGCUCAGUGU | |
| | UGUUUAAGAGAAUCAAAAGUUCUUAUGGUU | |
| | UGGUCUGGGAUCAAUAGGGAAACACAGGUAG | |
| | CCAACUAGGAGGAAAUGUACUGAAUGCUAGU | |
| | ACCCAAGACCUUGAGCAGGAAAGUCACCCAG | |
| | ACACCUCUGCUUUCUUUUGCCAUCUGACCUG | |
| | CAGCACUGUCAGGACAUGGCCUGUGGCUGUG | |
| | UGUUCAAACACCCCUCCCACAGGACUCACUU | |
| | UGUCCCAACAAUUCAGAUUGCCUAGAAAUAC | |
| | CUUUCUCUUACCUGUUUGUUAUUUAUCAAUU | |
| | UUUCCCAGUAUUUUUAUACGGAAAAAAUUGU | |
| | AUUGAAGACACUUUGUAUGCAGUUGAUAAG | |
| | AGGAAUUCAGUAUAAUUAUGGUUGGUGAUU | |
| | AUUUUUAUAAGCACAUGCCAACGCUUUACUA | |
| | CUGUGGAAAGACAAGUGUUUUAAUAAAAAG | |
| | AUUUACAUUCCAUGAUGUGGACGUCAUUUCU | |
| | UUUUUUUUUUAACAUCAUGUGUUUGGAGAG | |
| 142 | CAACGUCUAGGAUGUGAAGUUUGAAGAUUUC | MOUSE RETINOL BINDING |
| | UGAUUAGCUUUCAUCCGGUCUUCAUCUCUAU | PROTEIN 4, PLASMA |
| | UUAUCUUAGAAGUUUAGUUUCCCCCACCUCC | (RBP4) |
| | CCUACCUUCUCUAGGUGGACAUUAAACCAUC | |
| | GUCCAAAGUACAUGAGAGUCACUGACUCUGU | |
| | UCACACAACUGUAUGUCUUACUGAAGGUCCC | |
| | UGAAAGAUGUUUGAGGCUUGGGAUUCCAAAC | |
| | UUGGUUUAUUAAACAUAUAGUCACCAUCUUC | |
| | CUAU | |
| 143 | GCCCAUCACCCCACCUGGGUGGCUGGCAUUC | MOUSE PHOSPHOLIPID |
| | AGGAACCUAACUGAAGUCUUCUCUGCACCCC | TRANSFER PROTEIN |
| | CUGCCAACCCCUUCCCAUCUACAGUGUUAGU | (PLTP) |
| | GGUCCCGGUGCCACAGAGAAGAGCCCAGUUG | |
| | GAAGCUAUACCCGAUUUAAUUCCAGAAUUAG | |
| | UCAACCAUCAAUUAGAAUCCAUCCACCCCCC | |
| | UC | |
| 144 | GCAUCCUCUCACCAGACUAUGCCCUCCUGGA | MOUSE ALANINE- |
| | GGGGCUGGGAAUAUAGCAAGAACGAAAAGAC | GLYOXYLATE |
| | UGUGCAAGGCCUAGAGCCAGCAAAGAUGCUG | AMINOTRANSFERASE |
| | AUGUAGCCAGGCCAUGCCGGAAGGAGCAGGG | (AGXT) |
| | UGAAGCUUCCCCUCUCCCUACAAAUGGAACC | |
| | UUGUGGAAACAGGAUGCUAAACACCUUCUGA | |
| | UGGAGCUGUUGCCUGCAGGCCACUGGUCUUU | |
| | GGGAAUUUUCAAUAAAGUGCUUGCGAGGAA | |
| | UCUCCUA | |
| 145 | AGCCAAGACUGUGAUACUUCUCCUGUACCCU | ALDEHYDE |
| | GUUGACCUCAGGGAGUGCUGACCCUGUCUGG | DEHYDROGENASE 1 |
| | UGACUUAGCACCCUCCUGUCCCCAGCACUGC | FAMILY, MEMBER L1 |
| | UCCUUUCAGCUGCUGGAGCUCUUGGCCUGGA | (ALDHIL1) |
| | CCCCUGCUGGUGACAGGACACCCUCUGAACA | |
| | AUCAGAAGUGGCUCCAAGUGGAGUGAGCAGU | |
| | CAUGUCCCCCAUGAAUAAAAAUUGUGAGCAG | |
| | AGGUCGCCUACAAAAAAAAAAAAAAA | |
| 146 | AGCUCCGGAAGUCACAAGACACACCCUUGCC | FUMARYLACETOACETATE |
| | UUAUGAGGAUCAUGCUACCACUGCAUCAGUC | HYDROLASE (FAH) |
| | AGGAAUGAAUAAAGCUACUUUGAUUGUGGG | |
| | AAAUGCCACAGAAAAAAAAAAAAAAA | |
| 147 | AGGCCAGCCUUGCCCCUGCCCCAGAGCAGAG | FRUCTOSE |
| | CUCAAGUGACGCUACUCCAUUCUGCAUGUUG | BISPHOSPHATASE 1 (FBP1) |
| | UACAUUCCUAGAAACAAACCUAACAGCGUGG | |
| | AUAGUUUCACAGCUUAAUGCUUUGCAAUGCC | |
| | CAAGGUCACUUCAUCCUCAUGCUAUAAUGCC | |
| | ACUGUAUCAGGUAAUAUAUAUUUUGAGUAG | |
| | GUGAAGGAGAAAUAAACACAUCUUUCCUUUA | |
| | UAAAUUA | |
| 148 | GUUUCUCCGGCUCCCAGAAGCCCAUGCUCAG | MOUSE GLYCINE N- |
| | GCAAUGGCCCCUACCCUAAGACCAUCCCCUA | METHYLTRANSFERASE |
| | AUGCAGAUAUUGCAUUUGGGUGCAGAUGUG | (GNMT) |
| | GGGGUCGGGCAAACGGAGUAAACAAUACAGU | |
| | CUGCAUUCUCCAAAAAAAAAAAA | |
| 149 | GCCCCCAUCCACACAUGGACCACGCAAAGUG | MOUSE 4- |
| | CUGGACACAUCAGUCAUCUCCAACUGGCUGA | HYDROXYPHENYLPYRUVIC |
| | AAGGCUGAACCUCAGGGCUCCACCCACGUCA | ACID DIOXYGENASE |
| | UGGCCACGCCCCCUCUAUUACAAGAGUCCGC | (HPD) |
| | CUUGCCUGAGUCCUCCCUGCUGAGUAAAGCU | |
| | ACCCUCCCAGGUCCAAAAAAAAAAAAAAAAA | |
| | AAAAAAAAAAAAAAAAAAAAAAAAAAAAAA | |

### Triple Stop Codon

In some embodiments, RNA molecules provided herein, including self-replicating RNA and mRNA, may comprise a sequence immediately downstream of a coding region (i.e., ORF) that creates a triple stop codon. A triple stop codon is a sequence of three consecutive stop codons. The triple stop codon can ensure total insulation of an expression cassette and may be incorporated to enhance the efficiency of translation. In some embodiments, RNA molecules of the disclosure may comprise a triple combination of any of the sequences UAG, UGA, or UAA immediately downstream of an ORF described herein. The triple combination can be three of the same codons, three different codons, or any other permutation of the three stop codons.

### Translation Enhancers and Kozak Sequences

For translation initiation, proper interactions between ribosomes and mRNAs must be established to determine the exact position of the translation initiation region. However, ribosomes also must dissociate from the translation initiation region to slide toward the downstream sequence during mRNA translation. Translation enhancers upstream from initiation sequences of mRNAs enhance the yields of protein biosynthesis. Several studies have investigated the effects of translation enhancers. In some embodiments, an RNA molecule described herein, such as a self-replicating RNA or an mRNA, comprises a translation enhancer sequence. These translation enhancer sequences enhance the translation efficiency of a self-replicating RNA or mRNA of the disclosure and thereby provide increased production of the protein encoded by the RNA. The translation enhancer region may be located in the 5′ or 3′ UTR of a self-replicating RNA or an mRNA sequence. Examples of translation enhancer regions include naturally-occurring enhancer regions from the TEV 5′ UTR and the Xenopus beta-globin 3′ UTR. Exemplary 5′ UTR enhancer sequences include but are not limited to those derived from mRNAs encoding human heat shock proteins (HSP) including HSP70-P2, HSP70-M1 HSP72-M2, HSP17.9 and HSP70-P1. Exemplary translation enhancer sequences used in accordance with the embodiments of the present disclosure are represented by SEQ ID NOs: 58-62, as shown in Table 5.

TABLE 5
5′ UTR Enhancers

| Name | Sequence | Seq ID No.: |
|---|---|---|
| HSP70-P2 | GUCAGCUUUCAAACUCUUUGUUUCUUGUUUGUUGAUUG | 58 |
| | AGAAUA | |
| HSP70- | CUCUCGCCUGAGAAAAAAAAUCCACGAACCAAUUUCUCA | 59 |
| M1 | GCAACCAGCAGCACG | |
| HSP72- | ACCUGUGAGGGUUCGAAGGAAGUAGCAGUGUUUUUUGU | 60 |
| M2 | UCCUAGAGGAAGAG | |
| HSP17.9 | ACACAGAAACAUUCGCAAAAACAAAAUCCCAGUAUCAA | 61 |
| | AAUUCUUCUCUUUUUUUCAUAUUUCGCAAAGAC | |
| HSP70-P1 | CAGAAAAAUUUGCUACAUUGUUUCACAAACUUCAAAUA | 62 |
| | UUAUUCAUUUAUUU | |

In some embodiments, a self-replicating RNA or mRNA of the disclosure comprises a Kozak sequence. As is understood in the art, a Kozak sequence is a short consensus sequence centered around the translational initiation site of eukaryotic mRNAs that allows for efficient initiation of translation of the self-replicating RNA or mRNA. See, for example, Kozak, Marilyn (1988) Mol. and Cell Biol, 8:2737-2744; Kozak, Marilyn (1991) J. Biol. Chem, 266: 19867-19870; Kozak, Marilyn (1990) Proc Natl. Acad. Sci. USA, 87:8301-8305; and Kozak, Marilyn (1989) J. Cell Biol, 108:229-241. It ensures that a protein is correctly translated from the genetic message, mediating ribosome assembly and translation initiation. The ribosomal translation machinery recognizes the AUG initiation codon in the context of the Kozak sequence. A Kozak sequence may be inserted upstream of the coding sequence for the protein of interest, downstream of a 5′ UTR or inserted upstream of the coding sequence for the protein of interest and downstream of a 5′ UTR. In some embodiments, a self-replicating RNA or mRNA described herein comprises a Kozak sequence having the sequence GCCACC (SEQ ID NO: 63). A self-replicating RNA or mRNA described herein can comprise a partial Kozak sequence “p” having the nucleotide sequence GCCA (SEQ ID NO: 64).

### Antigenic Proteins

In some embodiments, nucleic acid molecules provided herein (e.g., RNA molecules, and polynucleotides encoding the same) can encode an antigenic protein or a fragment thereof. In some embodiments, the RNA molecules provided herein encode an entire HA polypeptide or an antigenic fragment thereof. In some embodiments, the RNA molecules provided herein encode an entire NA polypeptide or an antigenic fragment thereof. The RNA molecules provided herein may encode a homolog of any antigenic protein provided herein. Any antigenic protein can be encoded by nucleic acid molecules provided herein. In one aspect, the antigenic protein is a viral protein, a bacterial protein, a fungal protein, a protozoan protein, or a parasite protein. RNA molecules provided herein can be expressed from a subgenomic RNA derived from a self-replicating RNA or from an mRNA.

In some aspects, the antigenic protein, when administered to a mammalian subject, raises an immune response to a pathogen, optionally wherein the pathogen is viral, bacterial, fungal, protozoan, or any other type of pathogen. In other aspects, the antigenic protein is expressed on the outer surface of the pathogen; while in further aspects, the antigen may be a non-surface antigen, e.g., useful as a T-cell epitope. The immune response may comprise an antibody response (usually including IgG) and/or a cell mediated immune response. The polypeptide immunogen will typically elicit an immune response that recognizes the corresponding pathogen polypeptide, but in some embodiments, the polypeptide may act as a mimotope to elicit an immune response that recognizes a saccharide. The immunogen can be a surface polypeptide, e.g., an adhesin, a hemagglutinin, an envelope glycoprotein, a spike glycoprotein, etc.

Any viral, bacterial, fungal, protozoan, parasite, or other protein can be encoded by the RNA molecules provided herein. A protein from any infectious agent can be encoded by the RNA molecules provided herein. As used herein, the term “infectious agent” refers to any agent capable of infecting an organism, including humans and animals, and causing disease or deterioration in health. The terms “infectious agent” and “infectious pathogen” may be used interchangeably, unless context clearly indicates otherwise.

In one aspect, the antigenic protein encoded by nucleic acid molecules provided herein is an influenza virus protein or a fragment thereof. In another aspect, the nucleic acid molecules encode one or more influenza virus proteins or fragments thereof, from any strain or subtype of influenza virus. Exemplary influenza virus proteins that can be encoded by nucleic acid molecules provided herein include proteins from any human or animal virus, including influenza A virus, influenza B virus, influenza C virus, influenza D virus, or any combination thereof. Exemplary influenza proteins include hemagglutinin (HA), neuraminidase (NA), M2, M1, NP, NS1, NS2, PA, PB1, PB2, and PB1-F2. Hemagglutinin proteins from any influenza virus subtype, such as H1-H18 and any emerging hemagglutinin, and neuraminidase proteins from any influenza virus subtype, such as N1-N11 and any emerging neuraminidase, can be antigenic proteins encoded by the polynucleotides of nucleic acid molecules provided herein. Any suitable fragment of influenza virus proteins can be encoded by the polynucleotides of nucleic acid molecules provided herein, including, for example, one or more helper T lymphocyte (HTL) epitope, one or more cytotoxic T lymphocyte (CTL) epitope, or any combination thereof.

In some aspects, the polynucleotides of nucleic acid molecules provided herein encode a reporter or a marker, including selectable markers. Reporters and markers can include fluorescent proteins, such as green fluorescent protein (GFP), red fluorescent protein (RFP), yellow fluorescent protein (YFP), luciferase enzymes, such as firefly and Renilla luciferases, and antibiotic selection markers, for example.

In some aspects, nucleic acid molecules provided herein comprise at least two transgenes (e.g., at least two transgenes in addition to one or more viral replication proteins). Any number of transgenes can be included in nucleic acid molecules provided herein, such as one, two, three, four, five, six, seven, eight, nine, ten, or more transgenes. In one aspect, a polynucleotide of nucleic acid molecules provided herein encode an antigenic protein or a fragment thereof or an immunomodulatory protein. In one aspect, the nucleic acid molecule further comprises an internal ribosomal entry site (IRES), a sequence encoding a 2A peptide, or a combination thereof, located between transgenes. As used herein, the term “2A peptide” refers to a small (generally 18-22 amino acids) sequence that allows for efficient, stoichiometric production of discrete protein products within a single reading frame through a ribosomal skipping event within the 2A peptide sequence. As used herein, the term “internal ribosomal entry site” or “IRES” refers to a nucleotide sequence that allows for the initiation of protein translation of a messenger RNA (mRNA) sequence in the absence of an AUG start codon or without using an AUG start codon. An IRES can be found anywhere in an mRNA sequence, such as at or near the beginning, at or near the middle, or at or near the end of the mRNA sequence, for example. In another aspect, the nucleic acid molecules provided herein comprise one or more subgenomic promoter (e.g., upstream of each HA or NA encoded by the nucleic acid molecule). A subgenomic promoter located between transgenes can be a further subgenomic promoter, such as a second, third, fourth, etc. subgenomic promoter located between second and third, third and fourth, fourth and fifth, etc. transgenes, for example.

Any number of transgenes included in nucleic acid molecules provided herein can be expressed via any combination of 2A peptide and IRES sequences. For example, a second transgene located 3′ of a first transgene can be expressed via a 2A peptide sequence or via an IRES sequence. As another example, a second transgene located 3′ of a first transgene and a third transgene located 3′ of the second transgene can be expressed via 2A peptide sequences located between the first and second transgenes and the second and third transgenes, via an IRES sequence located between the first and second transgenes and the second and third transgenes, via a 2A peptide sequence located between the first and second transgenes and an IRES located between the second and third transgenes, or via an IRES sequence located between the first and second transgenes and a 2A peptide sequence located between the second and third transgenes. Similar configurations and combinations of 2A peptide and IRES sequences located between transgenes are contemplated for any number of transgenes included in polynucleotides of nucleic acid molecules provided herein. In addition to expression via 2A peptide and IRES sequences, two or more transgenes included in nucleic acid molecules provided herein can also be expressed from separate subgenomic RNAs.

Polynucleotides of nucleic acid molecules provided herein can encode an immunomodulatory protein or a functional fragment or functional variant thereof. Any immunomodulatory protein or a functional fragment or functional variant thereof can be encoded by polynucleotides.

As used herein, the terms “functional variant” or “functional fragment” refer to a molecule, including a nucleic acid or protein, for example, that comprises a nucleotide and/or amino acid sequence that is altered by one or more nucleotides and/or amino acids compared to the nucleotide and/or amino acid sequences of the parent or reference molecule. For a protein, a functional variant is still able to function in a manner that is similar to the parent molecule. In other words, the modifications in the amino acid and/or nucleotide sequence of the parent molecule do not significantly affect or alter the functional characteristics of the molecule encoded by the nucleotide sequence or containing the amino acid sequence. The functional variant may have conservative sequence modifications including nucleotide and amino acid substitutions, additions and deletions. These modifications can be introduced by standard techniques known in the art, such as site-directed mutagenesis and random PCR-mediated mutagenesis. Functional variants can also include, but are not limited to, derivatives that are substantially similar in primary structural sequence, but which contain, e.g., in vitro or in vivo modifications, chemical and/or biochemical, that are not found in the parent molecule. Such modifications include, inter alia, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI-anchor formation, hydroxylation, iodination, methylation, myristoylation, oxidation, pegylation, proteolytic processing, phosphorylation, prenylation, racemization, selenoylation, sulfation, transfer-RNA-mediated addition of amino acids to proteins such as arginylation, ubiquitination, and the like.

In one aspect, a transgene included in nucleic acid molecules provided herein encodes a cytokine, a chemokine, or an interleukin. Transgenes encoding a cytokine, a chemokine, or an interleukin can be included in nucleic acid molecules provided herein in addition to transgenes encoding HA, NA, both HA and NA, any other antigenic protein, or any combination thereof. Exemplary cytokines include interferons, TNF-α, TGF-β, G-CSF, and GM-CSF. Exemplary chemokines include CCL3, CCL26, and CXCL7. Exemplary interleukins include IL-I, IL-2, IL-3, IL-4, IL-5, IL-6, IL-7, IL-8, IL-10, IL-12, IL-15, IL-18, IL-21, and IL-23. Any transgene or combination of transgenes encoding any cytokine, chemokine, interleukin, or combinations thereof, can be included in nucleic acid molecules provided herein.

In one aspect, first and second transgenes included in nucleic acid molecules provided herein encode viral proteins, bacterial proteins, fungal proteins, protozoan proteins, parasite proteins, immunomodulatory proteins, or any combination thereof. In yet another aspect, first, second, third, fourth, fifth, sixth, seventh, eighth, ninth, tenth, or more transgenes included in polynucleotides of nucleic acid molecules provided herein encode viral proteins, bacterial proteins, fungal proteins, protozoan proteins, parasite proteins, immunomodulatory proteins, or any combination thereof.

In other aspects, the second transgene encodes a second influenza virus protein. In still other aspects, the first and second transgenes both encode an influenza virus protein, which may be from the same strain of influenza virus.

### RNA and DNA Molecules

### RNA Molecules—Exemplary Features

Nucleic acid molecules provided herein can be DNA molecules or RNA molecules. It will be appreciated that T present in DNA is substituted with U in RNA, and vice versa. In one aspect, nucleic acid molecules provided herein are RNA molecules, wherein a first polynucleotide is located 5′ of a second polynucleotide (which second polynucleotide may optionally be located 5′ of a third polynucleotide, and so on). Sequences presented herein as RNA sequences may be encoded by a corresponding DNA sequence, in which U is replaced with T. Similarly, sequences presented herein as DNA sequences may be converted to the corresponding RNA sequence encoded thereby by replacing T with U. In general, the RNA sequence “encoded” by a DNA sequence refers herein to an RNA with the same 5′ to 3′ orientation and order of bases (except for U replacing T), and not the reverse complement. Both the DNA and RNA versions of a given sequence are contemplated herein, unless context clearly indicates otherwise. In some cases, a DNA sequence may include elements not found in an RNA encoded thereby (e.g., a promoter sequence). In some cases, an RNA sequence may include elements not found in a DNA construct encoding the RNA (e.g., a poly-A tail when added post-transcriptionally).

RNA molecules provided herein can be self-replicating RNAs. In one aspect, RNA molecules provided herein include a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, or 100% identity to the sequence of SEQ ID NO:1, SEQ ID NO:2, SEQ ID NO:3, or SEQ ID NO:4. RNA molecules provided herein can also be mRNAs. It will be appreciated that T of sequences provided herein will be substituted with U in an RNA molecule.

An RNA molecule provided herein can be generated by in vitro transcription (IVT) of DNA molecules provided herein. In one aspect, RNA molecules provided herein are self-replicating RNA molecules. In another aspect, RNA molecules provided herein are mRNA molecules. In yet another aspect, RNA molecules provided herein further comprise a 5′ cap. Any 5′ cap can be included in RNA molecules provided herein, including 5′ caps having a Cap 1 structure, a Cap 1 (m6A) structure, a Cap 2 structure, or a Cap 0 structure. A population or plurality of RNA molecules provided herein can have the same 5′ cap or can have different 5′ caps. For example, a population or plurality of RNA molecules can have 5′ caps having a Cap 1 structure, a Cap 1 (m6A) structure, a Cap 2 structure, a Cap 0 structure, or any combination thereof.

In one aspect, RNA molecules provided herein include a 5′ cap having Cap 1 structure. In yet another aspect, RNA molecules provided herein are self-replicating RNA molecules comprising a 5′ cap having a Cap 1 structure. In a further aspect, RNA molecules provided herein comprise a cap having a Cap 1 structure, wherein a m7G is linked via a 5′-5′ triphosphate to the 5′ end of the 5′ UTR. In yet a further aspect, RNA molecules provided herein comprise a cap having a Cap 1 structure, wherein a m7G is linked via a 5′-5′ triphosphate to the 5′ end of the 5′ UTR comprising the sequence of SEQ ID NO:14. Any method of capping can be used, including, but not limited to using a Vaccinia Capping enzyme (New England Biolabs, Ipswich, Mass.) and co-transcriptional capping or capping at or shortly after initiation of in vitro transcription (IVT), by for example, including a capping agent as part of an in vitro transcription (IVT) reaction. (Nuc. Acids Symp. (2009) 53:129).

Only those RNA molecules, such mRNAs and self-replicating RNAs that can function as mRNAs, that carry the Cap structure are active in Cap dependent translation; “decapitation” of mRNA results in an almost complete loss of their template activity for protein synthesis (Nature, 255:33-37, (1975); J. Biol. Chem., vol. 253:5228-5231, (1978); and Proc. Natl. Acad. Sci. USA, 72:1189-1193, (1975)).

Another element of eukaryotic mRNA is the presence of 2′-O-methyl nucleoside residues at transcript position 1 (Cap 1), and in some cases, at transcript positions 1 and 2 (Cap 2). The 2′-O-methylation of mRNA provides higher efficacy of mRNA translation in vivo (Proc. Natl. Acad. Sci. USA, 77:3952-3956 (1980)) and further improves nuclease stability of the 5′-capped mRNA. The mRNA with Cap 1 (and Cap 2) is a distinctive mark that allows cells to recognize the bona fide mRNA 5′ end, and in some instances, to discriminate against transcripts emanating from infectious genetic elements (Nucleic Acid Research 43: 482-492 (2015)).

Some examples of 5′ cap structures and methods for preparing mRNAs comprising the same are given in WO2015/051169A2, WO/2015/061491, US 2018/0273576, and U.S. Pat. Nos. 8,093,367, 8,304,529, and 10,487,105. In some embodiments, the 5′ cap is m7GpppAmpG, which is known in the art. In some embodiments, the 5′ cap is m7GpppG or m7GpppGm, which are known in the art. Structural formulas for embodiments of 5′ cap structures are provided below.

In some embodiments, a self-replicating RNA or mRNA of the disclosure comprises a 5′ cap having the structure of Formula (Cap I).

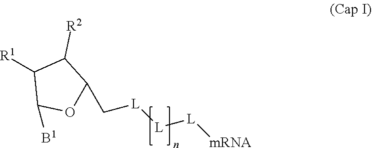

wherein B¹ is a natural or modified nucleobase; R¹ and R² are each independently selected from a halogen, OH, and OCH₃; each L is independently selected from the group consisting of phosphate, phophorothioate, and boranophosphate wherein each L is linked by diester bonds; n is 0 or 1. and mRNA represents an mRNA of the present disclosure linked at its 5′ end. In some embodiments B¹ is G, m⁷G, or A. In some embodiments, n is 0. In some embodiments n is 1. In some embodiments, B¹ is A or m⁶A and R¹ is OCH₃; wherein G is guanine, m⁷G is 7-methylguanine, A is adenine, and m⁶A is N⁶-methyladenine.

In some embodiments, a self-replicating RNA or mRNA of the disclosure comprises a 5′ cap having the structure of Formula (Cap II).

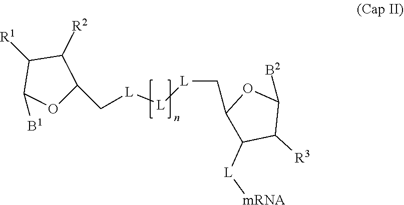

wherein B¹ and B² are each independently a natural or modified nucleobase; R¹, R², and R³ are each independently selected from a halogen, OH, and OCH₃; each L is independently selected from the group consisting of phosphate, phophorothioate, and boranophosphate wherein each L is linked by diester bonds; mRNA represents an mRNA of the present disclosure linked at its 5′ end; and n is 0 or 1. In some embodiments B¹ is G, m⁷G, or A. In some embodiments, n is 0. In some embodiments, n is 1. In some embodiments, B¹ is A or m⁶A and R¹ is OCH₃; wherein G is guanine, m⁷G is 7-methylguanine, A is adenine, and m⁶A is N⁶-methyladenine.

In some embodiments, a self-replicating RNA or mRNA of the disclosure comprises a 5′ cap having the structure of Formula (Cap III).

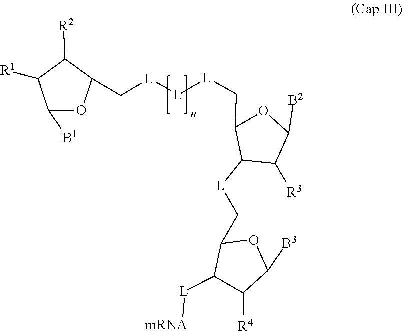

wherein B1, B2, and B3 are each independently a natural or modified nucleobase; R1, R2, R3, and R4 are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R1, R2, R3, and R4 is OH. In some embodiments B1 is G, m7G, or A. In some embodiments, B1 is A or m6A and R1 is OCH₃; wherein G is guanine, m7G is 7-methylguanine, A is adenine, and m6A is N6-methyladenine. In some embodiments, n is 1.

In some embodiments, a self-replicating RNA or mRNA of the disclosure comprises a m7GpppG 5′ cap analog having the structure of Formula (Cap IV).

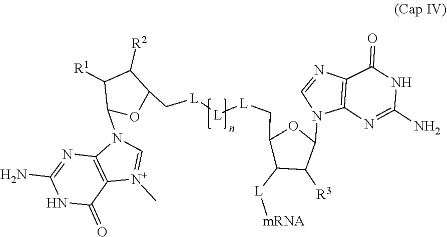

wherein, R¹, R², and R³ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; n is 0 or 1. In some embodiments, at least one of R¹, R², and R³ is OH. In some embodiments, the 5′ cap is m⁷GpppG wherein R¹, R², and R³ are each OH, n is 1, and each L is a phosphate. In some embodiments, n is 1. In some embodiments, the 5′ cap is m7GpppGm, wherein R¹ and R² are each OH, R³ is OCH₃, each L is a phosphate, and n is 1.

In some embodiments, a self-replicating RNA or mRNA of the disclosure comprises a m7Gpppm7G 5′ cap analog having the structure of Formula (Cap V).

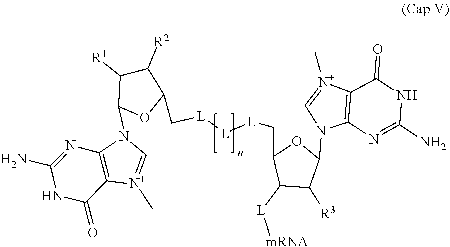

wherein, R¹, R², and R³ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², and R³ is OH. In some embodiments, n is 1.

In some embodiments, a self-replicating RNA or mRNA of the disclosure comprises a m7Gpppm7GpN, 5′ cap analog, wherein N is a natural or modified nucleotide, the 5′ cap analog having the structure of Formula (Cap VI).

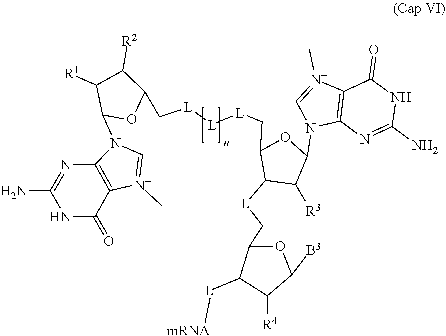

wherein B³ is a natural or modified nucleobase; R¹, R², R³, and R⁴ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 3. In some embodiments, at least one of R¹, R², R³, and R⁴ is OH. In some embodiments B¹ is G, m⁷G, or A. In some embodiments, B¹ is A or m⁶A and R¹ is OCH₃; wherein G is guanine, m⁷G is 7-methylguanine, A is adenine, and m′A is N′-methyladenine. In some embodiments, n is 1.

In some embodiments, a self-replicating RNA or mRNA of the disclosure comprises a m7Gpppm7GpG 5′ cap analog having the structure of Formula (Cap VII).

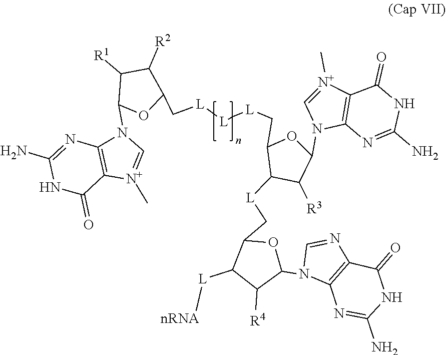

wherein, R¹, R², R³, and R⁴ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², R³, and R⁴ is OH. In some embodiments, n is 1.

In some embodiments, a self-replicating RNA or mRNA of the disclosure comprises a m7Gpppm7Gpm7G 5′ cap analog having the structure of Formula (Cap VIII).

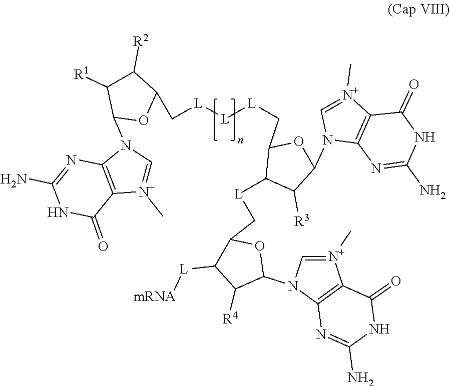

wherein, RH, R², R³, and R⁴ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; n is 0 or 1. In some embodiments, at least one of R¹, R², R³, and R⁴ is OH. In some embodiments, n is 1.

In some embodiments, a self-replicating RNA or mRNA of the disclosure comprises a m7GpppA 5′ cap analog having the structure of Formula (Cap IX).

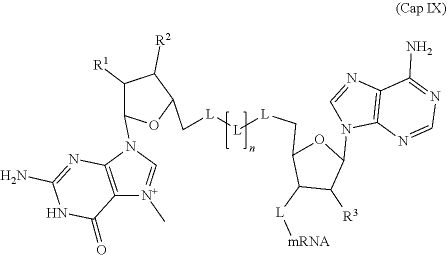

wherein, R¹, R², and R³ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², and R³ is OH. In some embodiments, n is 1.

In some embodiments, a self-replicating RNA or mRNA of the disclosure comprises a m7GpppApN 5′ cap analog, wherein N is a natural or modified nucleotide, and the 5′ cap has the structure of Formula (Cap X).

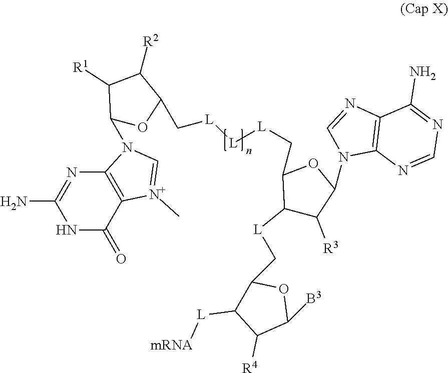

wherein B³ is a natural or modified nucleobase; R¹, R², R³, and R⁴ are each independently selected from a halogen, OH, and OCH₃; each L is independently selected from the group consisting of phosphate, phosphorothioate, and boranophosphate wherein each L is linked by diester bonds; miRNA represents an miRNA of the present disclosure linked at its 5′ end; and n is 0 or 1. In some embodiments, at least one of R¹, R², R³, and R⁴ is OH. In some embodiments B³ is G, m⁷G, A or m⁶A; wherein G is guanine, m⁷G is 7-methylguanine, A is adenine, and m⁶A is N⁶-methyladenine. In some embodiments, n is 1.

In some embodiments, a self-replicating RNA or mRNA of the disclosure comprises a m7GpppAmpG 5′ cap analog having the structure of Formula (Cap XI).

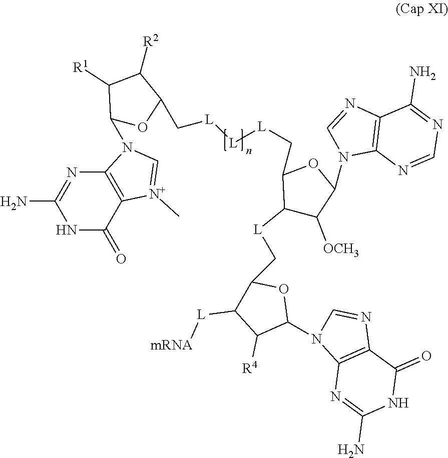

wherein, R¹, R², and R⁴ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², and R is OH. In some embodiments, the compound of Formula Cap XI is m⁷GpppAmpG, wherein R¹, R², and R⁴ are each OH, n is 1, and each L is a phosphate linkage. In some embodiments, n is 1.

In some embodiments, a self-replicating RNA or mRNA of the disclosure comprises a m7GpppApm7G 5′ cap analog having the structure of Formula (Cap XII).

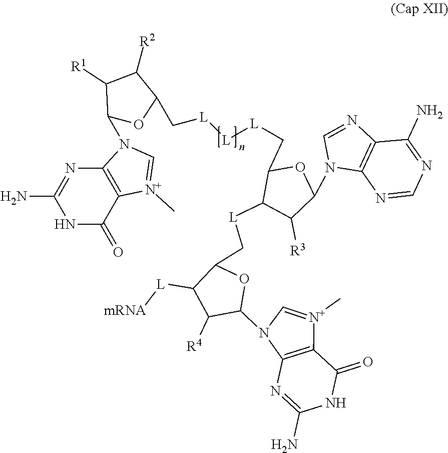

wherein, R¹, R², R³, and R⁴ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², R³, and R⁴ is OH. In some embodiments, n is 1.

In some embodiments, a self-replicating RNA or mRNA of the disclosure comprises a m7GpppApm7G 5′ cap analog having the structure of Formula (Cap XIII).

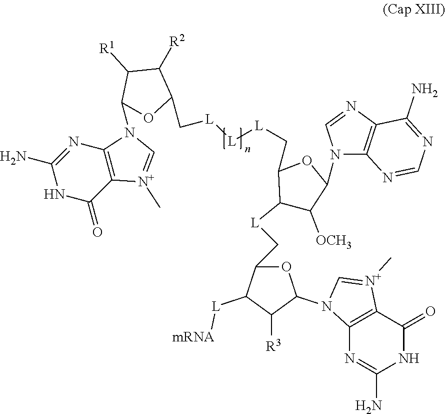

wherein, R¹, R², and R⁴ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², and R⁴ is OH. In some embodiments, n is 1.

### Poly-Adenine (Poly-A) Tail

Polyadenylation is the addition of a poly(A) tail, a chain of adenine nucleotides usually about 100-120 monomers in length, to a mRNA or an RNA that can function as an mRNA. In eukaryotes, polyadenylation is part of the process that produces mature mRNA for translation and begins as the transcription of a gene terminates. The 3′-most segment of a newly made pre-mRNA is first cleaved off by a set of proteins; these proteins then synthesize the poly(A) tail at the 3′ end. The poly(A) tail is important for the nuclear export, translation, and stability of mRNA. The tail is shortened over time, and, when it is short enough, the mRNA is enzymatically degraded. However, in a few cell types, mRNAs with short poly(A) tails are stored for later activation by re-polyadenylation in the cytosol. In some embodiments, the poly-A tail is transcribed from a template polynucleotide (e.g, the template incoding the entire saRNA), or is added post-transcriptionally. In some embodiments, an RNA molecule of the present disclosure comprises a poly-A tail encoded by the sequence of SEQ ID NO: 16. In some embodiments, the poly-A tail is encoded by a polynucleotide having the sequence of nucleotides 31-130 of SEQ ID NO: 16. In some embodiments, the poly-A tail is less than 100 nucleotides in length (e.g., 20-99 nucleotides in length). In some embodiments, the poly-A tail is less than 90, 80, 75, 70, 65, 60, 55, or 50 nucleotides in length.

In some embodiments, an RNA molecule of the disclosure comprises a 3′ tail region, which can serve to protect the RNA from exonuclease degradation. The tail region may be a 3′poly(A) and/or 3′poly(C) region. Preferably, the tail region is a 3′ poly(A) tail. Any self-replicating RNA and any mRNA, and any 3′ UTR of any self-replicating RNA or mRNA provided herein can include a poly(A) tail. As used herein a “3′ poly(A) tail” is a polymer of sequential adenine nucleotides that can range in size from, for example: 10 to 250 sequential adenine nucleotides; 60-125 sequential adenine nucleotides, 90-125 sequential adenine nucleotides, 95-125 sequential adenine nucleotides, 95-121 sequential adenine nucleotides, 100 to 121 sequential adenine nucleotides, 110-121 sequential adenine nucleotides; 112-121 sequential adenine nucleotides; 114-121 adenine sequential nucleotides; or 115 to 121 sequential adenine nucleotides. In some aspects, a 3′ poly(a) tail as described herein includes about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, 240, 250, 260, 270, 280, 290, 300, and any number or range in between, sequential adenine nucleotides. Preferably, a 3′ poly(A) tail as described herein comprises 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 sequential adenine nucleotides. In some embodiments, the 3′ poly(A) tail as described herein comprises 94 sequential adenine nucleotides. In some embodiments, the 3′ poly(A) tail as described herein comprises 106 sequential adenine nucleotides. In some embodiments, the 3′ poly(A) tail as described herein comprises 117 sequential adenine nucleotides. In some embodiments, the 3′ poly(A) tail as described herein comprises 141 sequential adenine nucleotides. 3′ Poly(A) tails can be added using a variety of methods known in the art, e.g., using poly(A) polymerase to add tails to synthetic or in vitro transcribed RNA. Other methods include the use of a transcription vector to encode poly(A) tails or the use of a ligase (e.g., via splint ligation using a T4 RNA ligase and/or T4 DNA ligase), wherein poly(A) may be ligated to the 3′ end of a sense RNA. In some embodiments, a combination of any of the above methods is utilized.

### DNA Molecules

In one aspect, provided herein are DNA molecules encoding the RNA molecules disclosed herein. In another aspect, DNA molecules provided herein further comprise a promoter. As used herein, the term “promoter” refers to a regulatory sequence that initiates transcription. A promoter can be operably linked to one or more polynucleotides of DNA molecules provided herein, with the one or more polynucleotides of DNA molecules corresponding to encoded one or more polynucleotides of RNA molecules provided herein. Generally, promoters included in DNA molecules provided herein include promoters for in vitro transcription (IVT). Any suitable promoter for in vitro transcription can be included in DNA molecules provided herein, such as a T7 promoter, a T3 promoter, an SP6 promoter, and others. In one aspect, DNA molecules provided herein comprise a T7 promoter. In another aspect, the promoter is located 5′ of the 5′ UTR included in DNA molecules provided herein. In yet another aspect, the promoter is a T7 promoter located 5′ of the 5′ UTR included in DNA molecules provided herein. In yet another aspect, the promoter overlaps with the 5′ UTR. A promoter and a 5′ UTR can overlap by about one nucleotide, about two nucleotides, about three nucleotides, about four nucleotides, about five nucleotides, about six nucleotides, about seven nucleotides, about eight nucleotides, about nine nucleotides, about ten nucleotides, about 11 nucleotides, about 12 nucleotides, about 13 nucleotides, about 14 nucleotides, about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides, about 30 nucleotides, about 31 nucleotides, about 32 nucleotides, about 33 nucleotides, about 34 nucleotides, about 35 nucleotides, about 36 nucleotides, about 37 nucleotides, about 38 nucleotides, about 39 nucleotides, about 40 nucleotides, about 41 nucleotides, about 42 nucleotides, about 43 nucleotides, about 44 nucleotides, about 45 nucleotides, about 46 nucleotides, about 47 nucleotides, about 48 nucleotides, about 49 nucleotides, about 50 nucleotides, or more nucleotides.

In some aspects, DNA molecules provided herein include a promoter for in vivo transcription. Generally, the promoter for in vivo transcription is an RNA polymerase II (RNA pol II) promoter. Any RNA pol II promoter can be included in DNA molecules provided herein, including constitutive promoters, inducible promoters, and tissue-specific promoters. Exemplary constitutive promoters include a cytomegalovirus (CMV) promoter, an EF1α promoter, an SV40 promoter, a PGK1 promoter, a Ubc promoter, a human beta actin promoter, a CAG promoter, and others. Any tissue-specific promoter can be included in DNA molecules provided herein. In one aspect, the RNA pol II promoter is a muscle-specific promoter, skin-specific promoter, subcutaneous tissue-specific promoter, liver-specific promoter, spleen-specific promoter, lymph node-specific promoter, or a promoter with any other tissue specificity. DNA molecules provided herein can also include an enhancer. Any enhancer that increases transcription can be included in DNA molecules provided herein.

### Design and Synthesis of RNA and DNA Molecules

RNA molecules provided herein can include any combination of the RNA sequences provided herein, including, for example, any 5′ UTR sequences, any sequences encoding a polyprotein that includes nsP1, nsP2, nsP3, and nsP4, any sequences encoding any transgene disclosed herein (e.g., antigenic influenza polypeptides or fragments thereof), and any 3′ UTR sequences provided herein. In some aspects, RNA molecules provided herein are self-replicating RNA molecules. Self-replicating RNA molecules can encode one or more viral replication proteins that include nsP1, nsP2, nsP3, and nsP4, for example. In some aspects, RNA molecules provided herein are mRNA molecules. Generally, mRNA molecules do not include sequences encoding a polyprotein for replication of the RNA.

In some aspects, RNA molecules provided herein include modified nucleotides. For example, 0% to 100%, 1% to 100%, 25% to 100%, 50% to 100% and 75% to 100% of the uracil nucleotides of the RNA molecules can be modified. In some aspects, 1% to 100% of the uracil nucleotides are N1-methylpseudouridine or 5-methoxyuridine. In some embodiments, 100% of the uracil nucleotides are N1-methylpseudouridine. In some embodiments, 100% of the uracil nucleotides are 5-methoxyuridine.

An RNA molecule, such as a self-replicating RNA or mRNA, of the disclosure may be obtained by any suitable means. Methods for the manufacture of RNA molecules are known in the art and would be readily apparent to a person of ordinary skill. An RNA molecule of the disclosure may be prepared according to any available technique including, but not limited to chemical synthesis, in vitro transcription (IVT) or enzymatic or chemical cleavage of a longer precursor, etc.

In some embodiments, an RNA molecule, such as a self-replicating RNA or mRNA, of the disclosure is produced from a primary complementary DNA (cDNA) construct. The cDNA constructs can be produced on an RNA template by the action of a reverse transcriptase (e.g., RNA-dependent DNA-polymerase). The process of design and synthesis of the primary cDNA constructs described herein generally includes the steps of gene construction, RNA production (either with or without modifications) and purification. In the IVT method, a target polynucleotide sequence encoding an RNA molecule of the disclosure is first selected for incorporation into a vector which will be amplified to produce a cDNA template. Optionally, the target polynucleotide sequence and/or any flanking sequences may be codon optimized. The cDNA template is then used to produce an RNA molecule of the disclosure through in vitro transcription (IVT). After production, the RNA molecule of the disclosure may undergo purification and clean-up processes. The steps of which are provided in more detail below.

The step of gene construction may include, but is not limited to gene synthesis, vector amplification, plasmid purification, plasmid linearization and clean-up, and cDNA template synthesis and clean-up. Once a protein of interest is selected for production, a primary construct is designed. Within the primary construct, a first region of linked nucleosides encoding the polypeptide of interest may be constructed using an open reading frame (ORF) of a selected nucleic acid (DNA or RNA) transcript. The ORF may comprise the wild type ORF, an isoform, variant or a fragment thereof. As used herein, an “open reading frame” or “OR” is meant to refer to a nucleic acid sequence (DNA or RNA) which can encode a polypeptide of interest. ORFs often begin with the start codon, ATG and end with a nonsense or termination codon or signal.

The cDNA templates may be transcribed to produce an RNA molecule of the disclosure using an in vitro transcription (IVT) system. The system typically comprises a transcription buffer, nucleotide triphosphates (NTPs), an RNase inhibitor and a polymerase. The NTPs may be selected from, but are not limited to, those described herein including natural and unnatural (modified) NTPs. The polymerase may be selected from, but is not limited to, T7 RNA polymerase, T3 RNA polymerase and mutant polymerases such as, but not limited to, polymerases able to incorporate modified nucleic acids.

The primary cDNA template or transcribed RNA sequence may also undergo capping and/or tailing reactions. A capping reaction may be performed by methods known in the art to add a 5′ cap to the 5′ end of the primary construct. Methods for capping include, but are not limited to, using a Vaccinia Capping enzyme (New England Biolabs, Ipswich, Mass.) or capping at initiation of in vitro transcription, by for example, including a capping agent as part of the IVT reaction. (Nuc. Acids Symp. (2009) 53:129). A poly(A) tailing reaction may be performed by methods known in the art, such as, but not limited to, 2′ O-methyltransferase and by methods as described herein. If the primary construct generated from cDNA does not include a poly-T, it may be beneficial to perform the poly(A)-tailing reaction before the primary construct is cleaned.

Codon optimized cDNA constructs encoding the non-structural proteins and the transgene for a self-replicating RNA are particularly suitable for generating self-replicating RNA sequences described herein. For example, such cDNA constructs may be used as the basis to transcribe, in vitro, a polyribonucleotide encoding a protein of interest as part of a self-replicating RNA. Codon optimized cDNA constructs can also be used to generate mRNAs provided herein.

The present disclosure also provides expression vectors comprising a nucleotide sequence encoding a self-replicating RNA or mRNA that is preferably operably linked to at least one regulatory sequence. Regulatory sequences are art-recognized and are selected to direct expression of the encoded polypeptide.

Accordingly, the term regulatory sequence includes promoters, enhancers, and other expression control elements. The design of the expression vector may depend on such factors as the choice of the host cell to be transformed and/or the type of protein desired to be expressed.

The present disclosure also provides polynucleotides (e.g. DNA, RNA, cDNA, mRNA, etc.) directed to a self-replicating RNA or mRNA of the disclosure that may be operably linked to one or more regulatory nucleotide sequences in an expression construct, such as a vector or plasmid. In certain embodiments, such constructs are DNA constructs. Regulatory nucleotide sequences will generally be appropriate for a host cell used for expression. Numerous types of appropriate expression vectors and suitable regulatory sequences are known in the art for a variety of host cells.

Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the embodiments of the present disclosure. The promoters may be either naturally occurring promoters, or hybrid promoters that combine elements of more than one promoter.

An expression construct may be present in a cell on an episome, such as a plasmid, or the expression construct may be inserted in a chromosome. In some embodiments, the expression vector contains a selectable marker gene to allow the selection of transformed host cells. Selectable marker genes are well known in the art and will vary with the host cell used.

The present disclosure also provides a host cell transfected with a self-replicating RNA, mRNA, or DNA described herein. The self-replicating RNA, mRNA, or DNA can encode any protein of interest, for example an antigen or any other viral glycoprotein, such as the influenza virus hemagglutinin and neuraminidase. The host cell may be any prokaryotic or eukaryotic cell. For example, a polypeptide encoded by a self-replicating RNA or mRNA may be expressed in bacterial cells such as E. coli, insect cells (e.g., using a baculovirus expression system), yeast, or mammalian cells. Other suitable host cells are known to those skilled in the art.

A host cell transfected with an expression vector comprising a self-replicating RNA or mRNA of the disclosure can be cultured under appropriate conditions to allow expression of the self-replicating RNA or mRNA and translation of the polypeptide to occur. Once expressed, a self-replicating RNA generally undergoes self-amplification and translation. The polypeptide may be secreted and isolated from a mixture of cells and medium containing the polypeptides. Alternatively, the polypeptides may be retained in the cytoplasm or in a membrane fraction and the cells harvested, lysed and the protein isolated. A cell culture includes host cells, media and other byproducts. Suitable media for cell culture are well known in the art.

The expressed proteins described herein can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptide.

### Compositions and Pharmaceutical Compositions

Provided herein, in some embodiments, are compositions comprising any of the RNA or DNA molecules provided herein. Compositions provided herein can include a lipid. In one aspect, the lipid is an ionizable cationic lipid. Any ionizable cationic lipid can be included in compositions comprising nucleic acid molecules provided herein.

The compositions, molecules and polynucleotides of the present disclosure may be used to immunize or vaccinate a subject against a viral infection, such as influenza. In some embodiments, the compositions, molecules and polynucleotides of the present disclosure may be used to vaccinate or immunize a subject against any or all of four distinct strains of influenza (e.g., two from influenza A subtypes, H1N1 and H3N2, and two from influenza B subtypes, Victoria and Yamagata).

Also provided herein, in some embodiments, are pharmaceutical compositions comprising any of the RNA and DNA molecules provided herein and a lipid formulation. Any lipid can be included in lipid formulations of pharmaceutical compositions provided herein. In one aspect, lipid formulations of pharmaceutical compositions provided herein include an ionizable cationic lipid. Exemplary ionizable cationic lipids of compositions and pharmaceutical compositions provided herein include the following in Table 6:

TABLE 6
Exemplary Ionizable Cationic Lipids

| | |
|---|---|
| 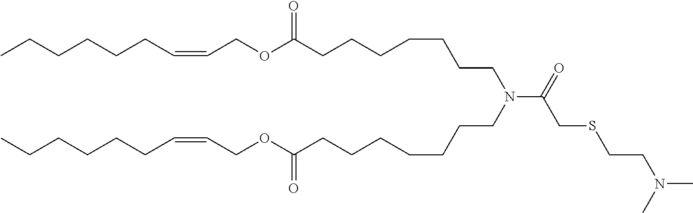 [SMILES: CCCCCC/C=C\COC(=O)CCCCCCCN(CCCCCCCC(=O)OC/C=C\CCCCCC)C(=O)CSCCN(C)C] | ATX-001 |
| 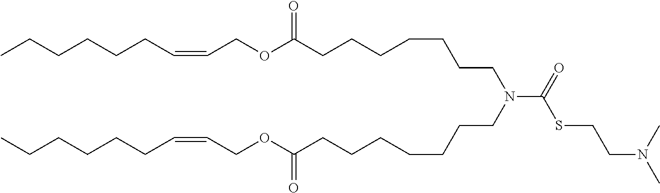 [SMILES: CCCCCC/C=C\COC(=O)CCCCCCCN(CCCCCCCC(=O)OC/C=C\CCCCCC)C(=O)SCCN(C)C] | ATX-002 |
| 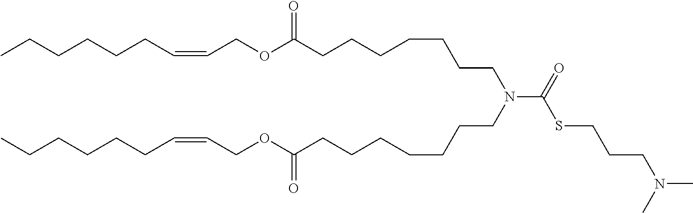 [SMILES: CCCCCC/C=C\COC(=O)CCCCCCCN(CCCCCCCC(=O)OC/C=C\CCCCCC)C(=O)SCCCN(C)C] | ATX-003 |
| 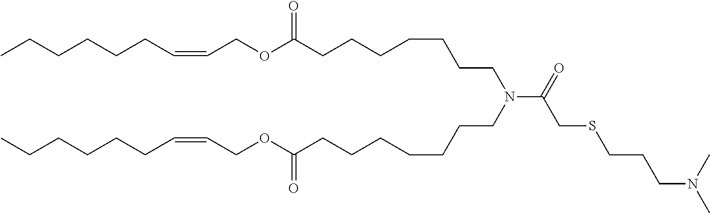 [SMILES: CCCCCC/C=C\COC(=O)CCCCCCCN(CCCCCCCC(=O)OC/C=C\CCCCCC)C(=O)CSCCCN(C)C] | ATX-004 |
| 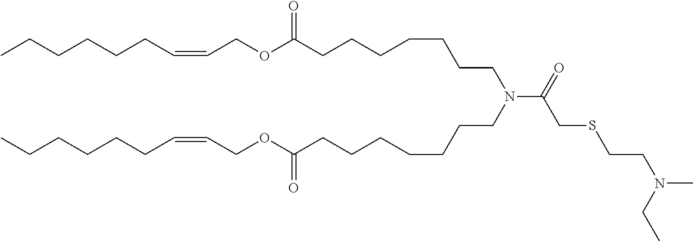 [SMILES: CCCCCC/C=C\COC(=O)CCCCCCCN(CCCCCCCC(=O)OC/C=C\CCCCCC)C(=O)CSCCN(C)CC] | ATX-005 |
| 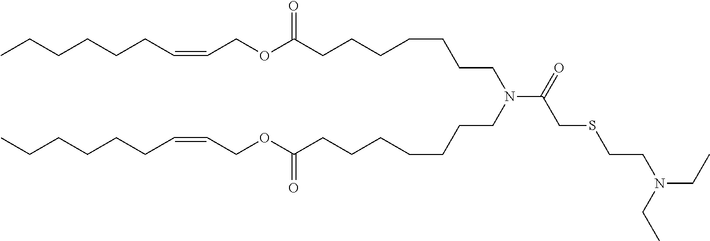 [SMILES: CCCCCC/C=C\COC(=O)CCCCCCCN(CCCCCCCC(=O)OC/C=C\CCCCCC)C(=O)CSCCN(CC)CC] | ATX-006 |
| 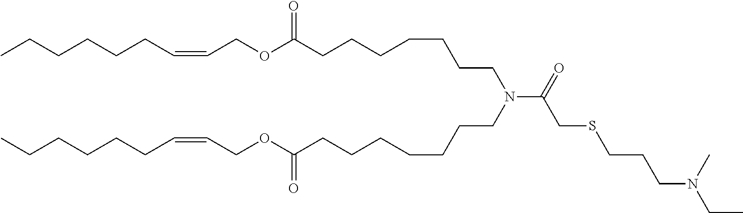 [SMILES: CCCCCC/C=C\COC(=O)CCCCCCCN(CCCCCCCC(=O)OC/C=C\CCCCCC)C(=O)CSCCCN(C)CC] | ATX-007 |
| 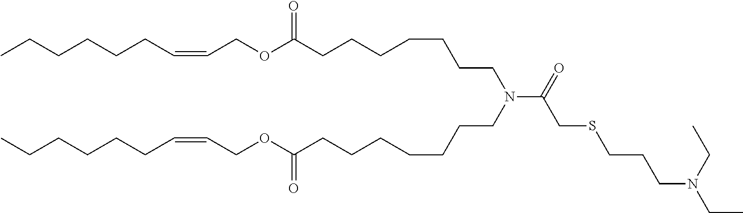 [SMILES: CCCCCC/C=C\COC(=O)CCCCCCCN(CCCCCCCC(=O)OC/C=C\CCCCCC)C(=O)CSCCCN(CC)CC] | ATX-008 |
| 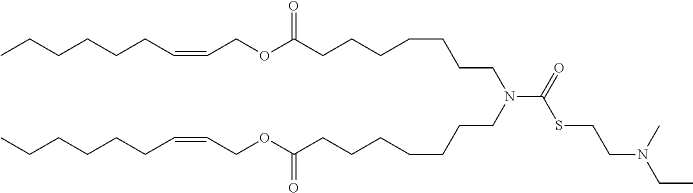 [SMILES: CCCCCC/C=C\COC(=O)CCCCCCCN(CCCCCCCC(=O)OC/C=C\CCCCCC)C(=O)SCCN(C)CC] | ATX-009 |
| 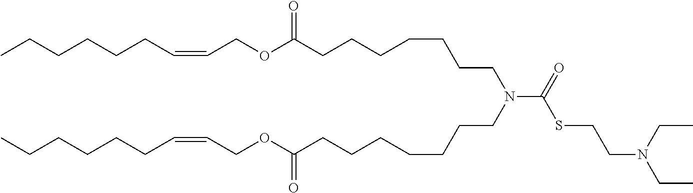 [SMILES: CCCCCC/C=C\COC(=O)CCCCCCCN(CCCCCCCC(=O)OC/C=C\CCCCCC)C(=O)SCCN(CC)CC] | ATX-010 |
| 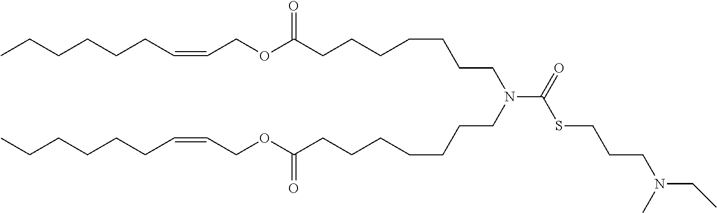 [SMILES: CCCCCC/C=C\COC(=O)CCCCCCCN(CCCCCCCC(=O)OC/C=C\CCCCCC)C(=O)SCCCN(C)CC] | ATX-011 |
| 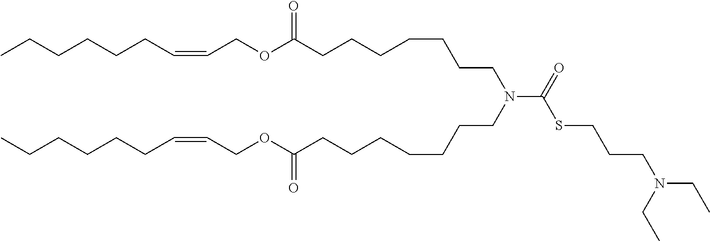 [SMILES: CCCCCC/C=C\COC(=O)CCCCCCCN(CCCCCCCC(=O)OC/C=C\CCCCCC)C(=O)SCCCN(CC)CC] | ATX-012 |
| 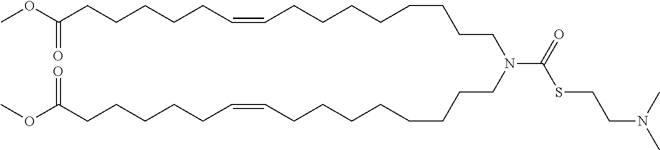 | ATX-013 |
| 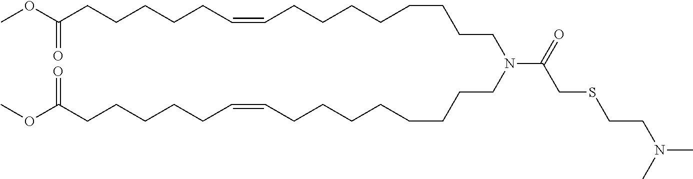 | ATX-014 |
| 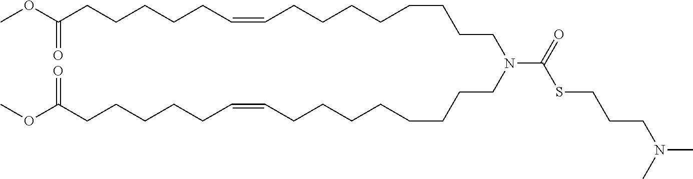 | ATX-015 |
| 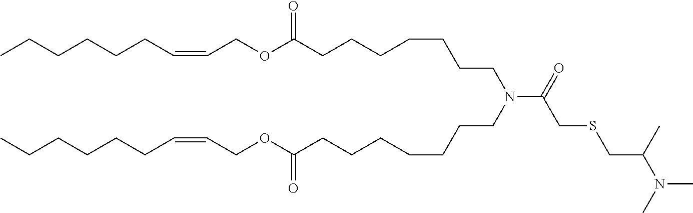 [SMILES: CCCCCC/C=C\COC(=O)CCCCCCCN(CCCCCCCC(=O)OC/C=C\CCCCCC)C(=O)CSCC(C)N(C)C] | ATX-016 |
| 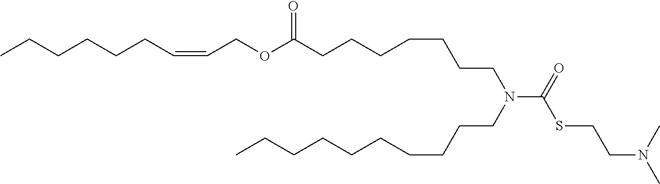 [SMILES: CCCCCC/C=C\COC(=O)CCCCCCCN(CCCCCCCCCC)C(=O)SCCN(C)C] | ATX-018 |
| 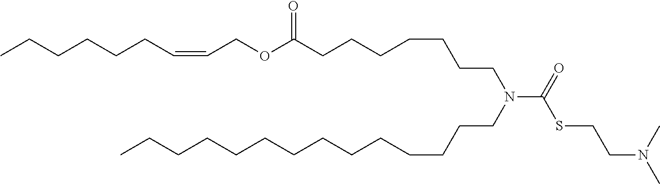 [SMILES: CCCCCC/C=C\COC(=O)CCCCCCCN(CCCCCCCCCCCCCC)C(=O)SCCN(C)C] | ATX-019 |
| 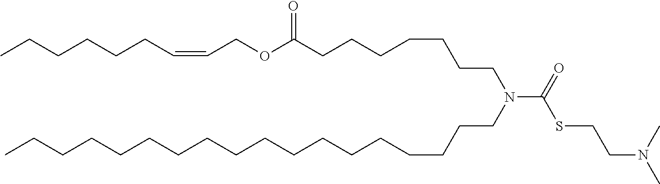 [SMILES: CCCCCC/C=C\COC(=O)CCCCCCCN(CCCCCCCCCCCCCCCCCC)C(=O)SCCN(C)C] | ATX-020 |
| 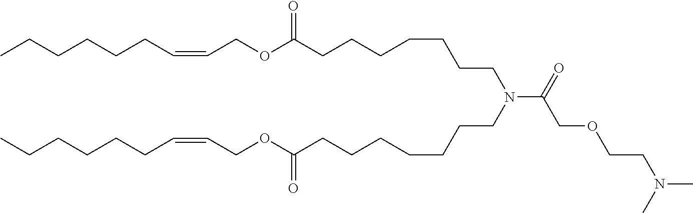 [SMILES: CCCCCC/C=C\COC(=O)CCCCCCCN(CCCCCCCC(=O)OC/C=C\CCCCCC)C(=O)COCCN(C)C] | ATX-021 |
| 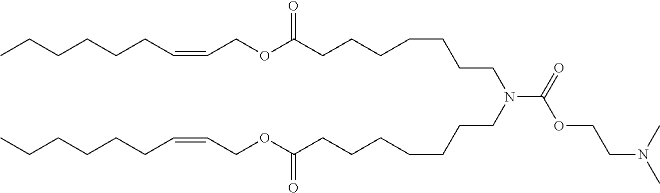 [SMILES: CCCCCC/C=C\COC(=O)CCCCCCCN(CCCCCCCC(=O)OC/C=C\CCCCCC)C(=O)OCCN(C)C] | ATX-022 |
| 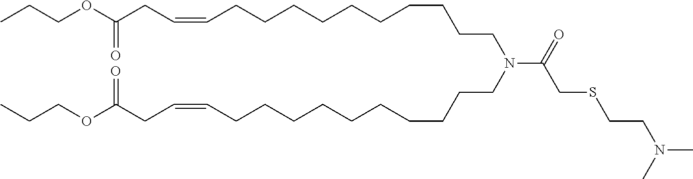 [SMILES: CCCOC(=O)C/C=C\CCCCCCCCCCN(CCCCCCCCCC/C=C\CC(=O)OCCC)C(=O)CSCCN(C)C] | ATX-023 |
| 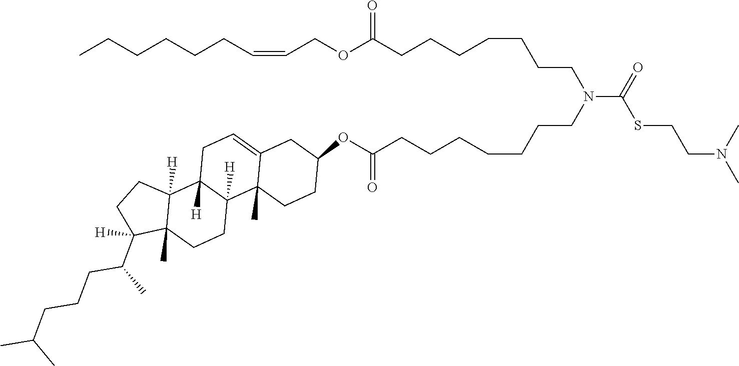 | ATX-024 |
| 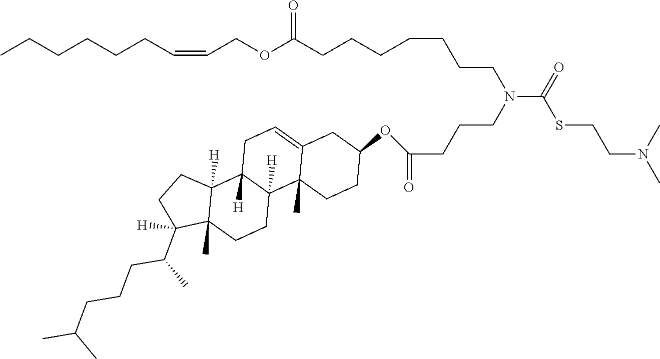 | ATX-025 |
| 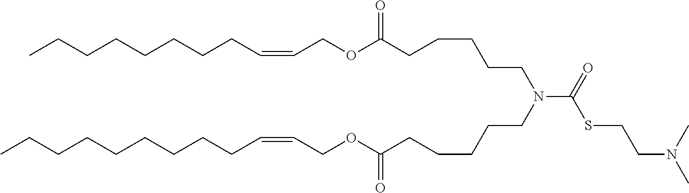 [SMILES: CCCCCCCC/C=C\COC(=O)CCCCCN(CCCCCC(=O)OC/C=C\CCCCCCCCC)C(=O)SCCN(C)C] | ATX-026 |
| 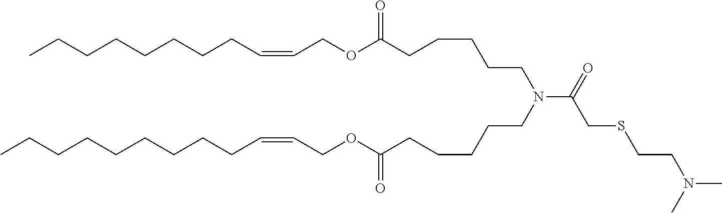 [SMILES: CCCCCCCC/C=C\COC(=O)CCCCCN(CCCCCC(=O)OC/C=C\CCCCCCCCC)C(=O)CSCCN(C)C] | ATX-027 |
| 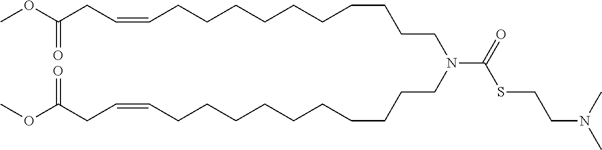 | ATX-028 |
| 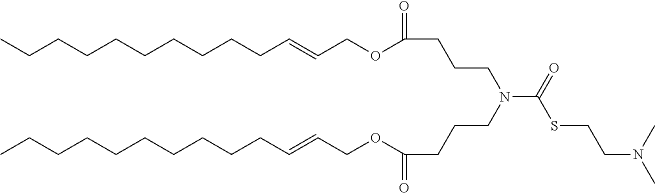 [SMILES: CCCCCCCCCC/C=C/COC(=O)CCCN(CCCC(=O)OC/C=C/CCCCCCCCCC)C(=O)SCCN(C)C] | ATX-029 |
| 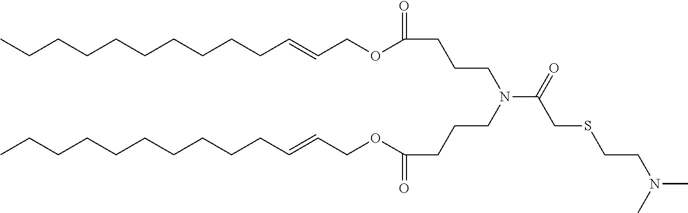 [SMILES: CCCCCCCCCC/C=C/COC(=O)CCCN(CCCC(=O)OC/C=C/CCCCCCCCCC)C(=O)CSCCN(C)C] | ATX-030 |
| 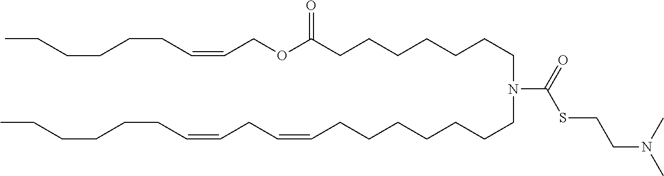 [SMILES: CCCCCC/C=C\C/C=C\CCCCCCCN(CCCCCCCC(=O)OC/C=C\CCCCCC)C(=O)SCCN(C)C] | ATX-031 |
| 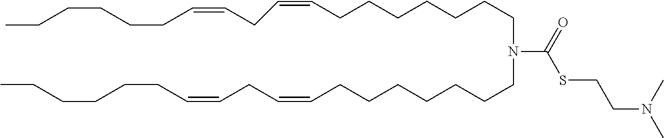 [SMILES: CCCCCC/C=C\C/C=C\CCCCCCCN(CCCCCCC/C=C\C/C=C\CCCCCC)C(=O)SCCN(C)C] | ATX-032 |
| 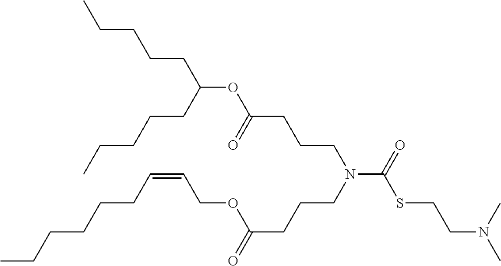 [SMILES: CCCCCC/C=C\COC(=O)CCCN(CCCC(=O)OC(CCCCC)CCCCC)C(=O)SCCN(C)C] | ATX-43 |
| 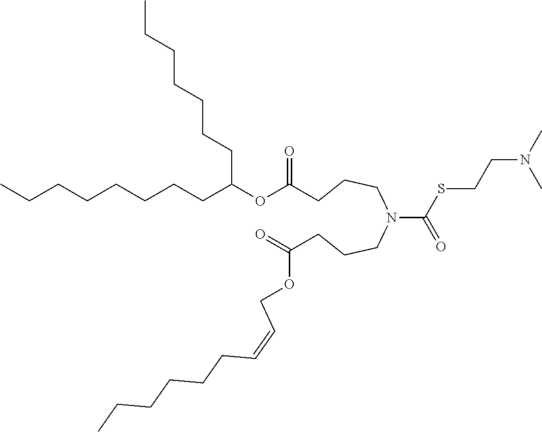 [SMILES: CCCCCC/C=C\COC(=O)CCCN(CCCC(=O)OC(CCCCCCC)CCCCCCCC)C(=O)SCCN(C)C] | ATX-057 |
| 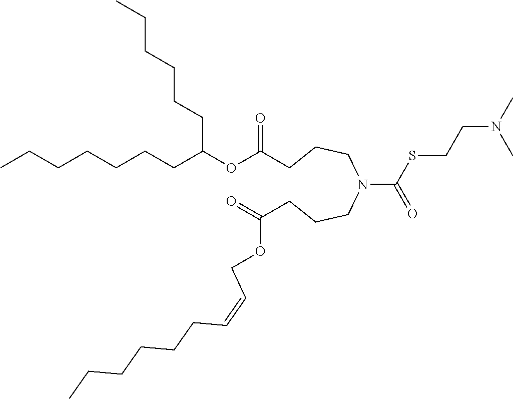 [SMILES: CCCCCC/C=C\COC(=O)CCCN(CCCC(=O)OC(CCCCCC)CCCCCCC)C(=O)SCCN(C)C] | ATX-058 |
| 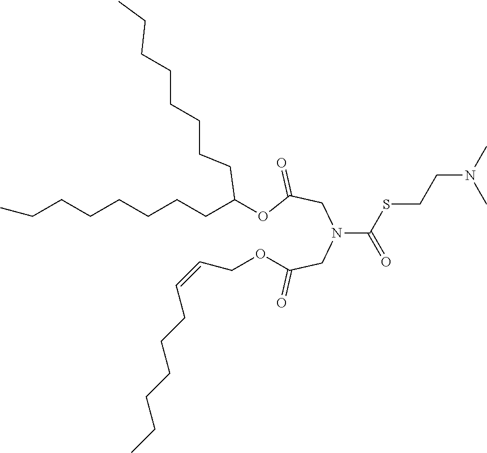 [SMILES: CCCCCC/C=C\COC(=O)CN(CC(=O)OC(CCCCCCCC)CCCCCCCC)C(=O)SCCN(C)C] | ATX-061 |
| 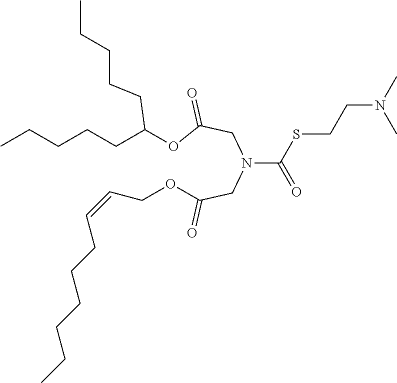 [SMILES: CCCCCC/C=C\COC(=O)CN(CC(=O)OC(CCCCC)CCCCC)C(=O)SCCN(C)C] | ATX-063 |
| 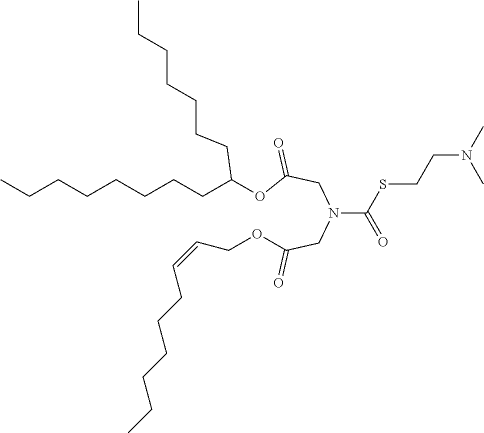 [SMILES: CCCCCC/C=C\COC(=O)CN(CC(=O)OC(CCCCCCC)CCCCCCCC)C(=O)SCCN(C)C] | ATX-064 |
| 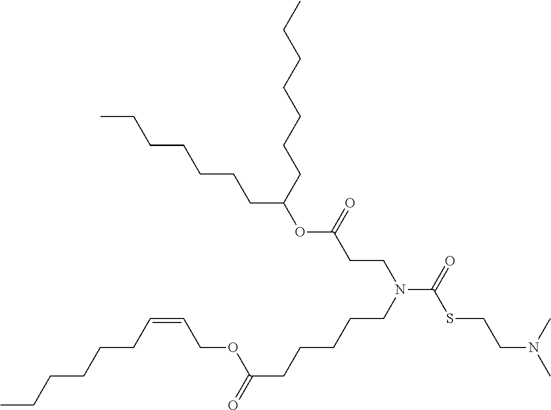 [SMILES: CCCCCC/C=C\COC(=O)CCCCCN(CCC(=O)OC(CCCCCCC)CCCCCCC)C(=O)SCCN(C)C] | ATX-082 |
| 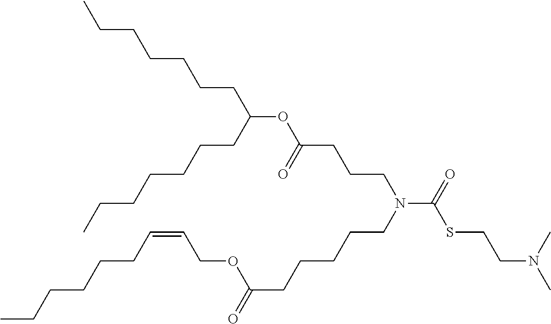 [SMILES: CCCCCC/C=C\COC(=O)CCCCCN(CCCC(=O)OC(CCCCCCC)CCCCCCC)C(=O)SCCN(C)C] | ATX-083 |
| 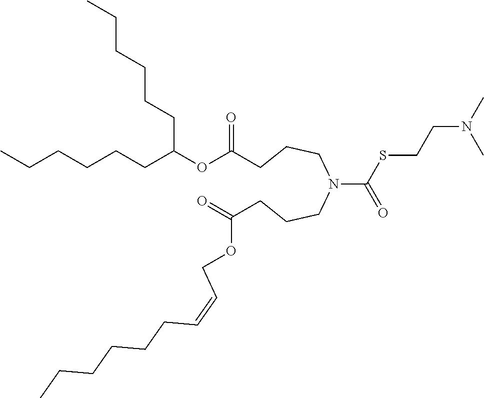 [SMILES: CCCCCC/C=C\COC(=O)CCCN(CCCC(=O)OC(CCCCCC)CCCCCC)C(=O)SCCN(C)C] | ATX-086 |
| 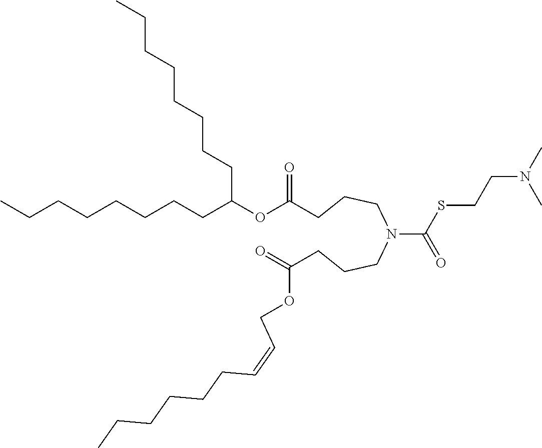 [SMILES: CCCCCC/C=C\COC(=O)CCCN(CCCC(=O)OC(CCCCCCCC)CCCCCCCC)C(=O)SCCN(C)C] | ATX-087 |
| 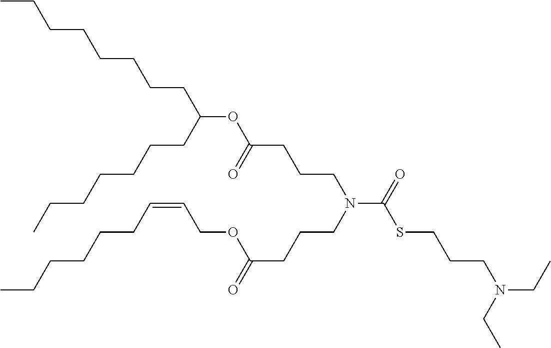 [SMILES: CCCCCC/C=C\COC(=O)CCCN(CCCC(=O)OC(CCCCCCC)CCCCCCCC)C(=O)SCCCN(CC)CC] | ATX-088 |
| 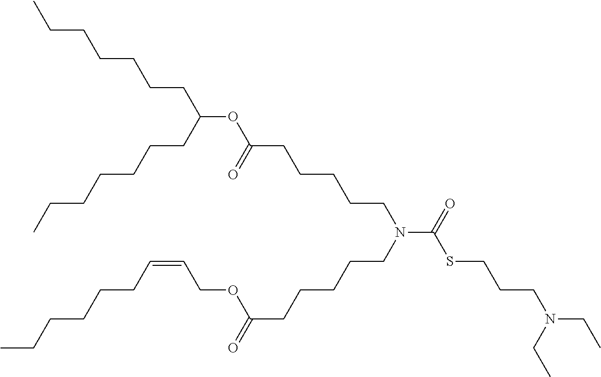 [SMILES: CCCCCC/C=C\COC(=O)CCCCCN(CCCCCC(=O)OC(CCCCCCC)CCCCCCC)C(=O)SCCCN(CC)CC] | ATX-109 |
| 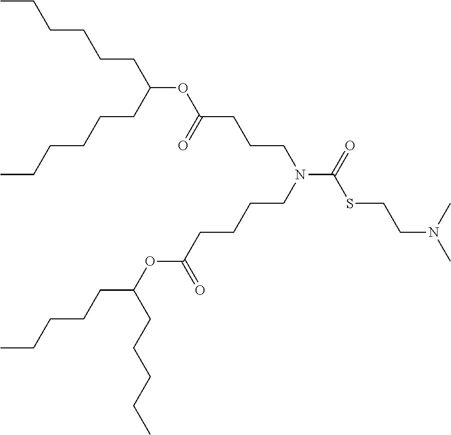 [SMILES: CCCCCCC(CCCCCC)OC(=O)CCCN(CCCCC(=O)OC(CCCCC)CCCCC)C(=O)SCCN(C)C] | ATX-085 |
| 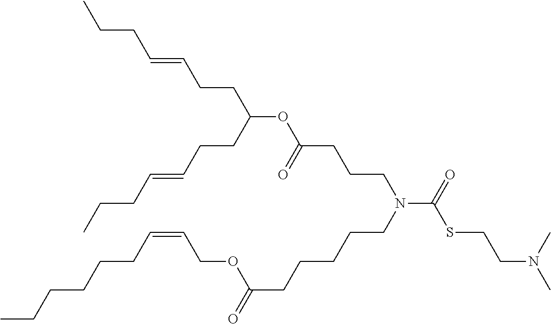 [SMILES: CCC/C=C/CCC(CC/C=C/CCC)OC(=O)CCCN(CCCCCC(=O)OC/C=C\CCCCCC)C(=O)SCCN(C)C] | ATX-0121 |
| 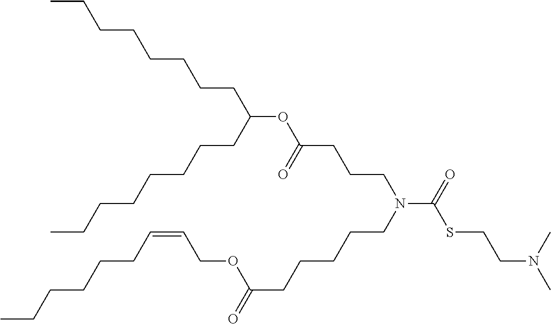 [SMILES: CCCCCC/C=C\COC(=O)CCCCCN(CCCC(=O)OC(CCCCCCCC)CCCCCCCC)C(=O)SCCN(C)C] | ATX-091 |
| 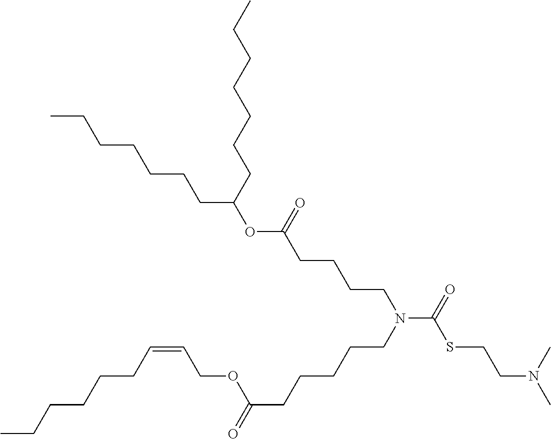 [SMILES: CCCCCC/C=C\COC(=O)CCCCCN(CCCCC(=O)OC(CCCCCCC)CCCCCCC)C(=O)SCCN(C)C] | ATX-0102 |
| 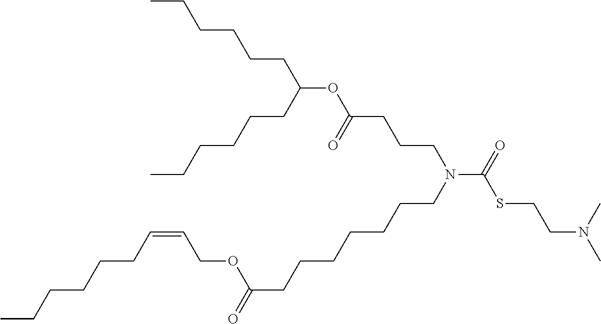 [SMILES: CCCCCC/C=C\COC(=O)CCCCCCCN(CCCC(=O)OC(CCCCCC)CCCCCC)C(=O)SCCN(C)C] | ATX-098 |
| 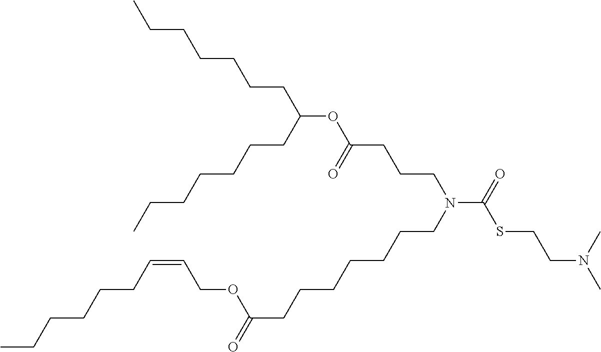 [SMILES: CCCCCC/C=C\COC(=O)CCCCCCCN(CCCC(=O)OC(CCCCCCC)CCCCCCC)C(=O)SCCN(C)C] | ATX-092 |
| 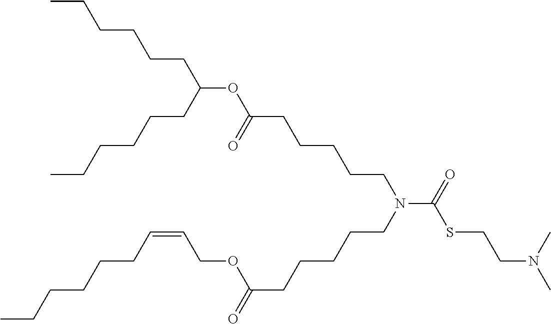 [SMILES: CCCCCC/C=C\COC(=O)CCCCCN(CCCCCC(=O)OC(CCCCCC)CCCCCC)C(=O)SCCN(C)C] | ATX-084 |
| 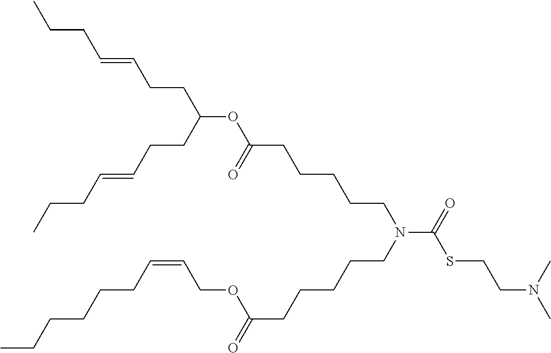 [SMILES: CCC/C=C/CCC(CC/C=C/CCC)OC(=O)CCCCCN(CCCCCC(=O)OC/C=C\CCCCCC)C(=O)SCCN(C)C] | ATX-0125 |
| 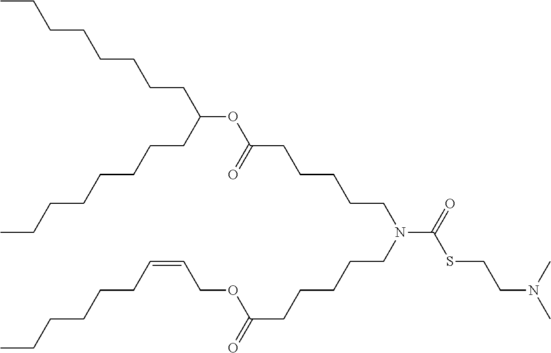 [SMILES: CCCCCC/C=C\COC(=O)CCCCCN(CCCCCC(=O)OC(CCCCCCCC)CCCCCCCC)C(=O)SCCN(C)C] | ATX-094 |
| 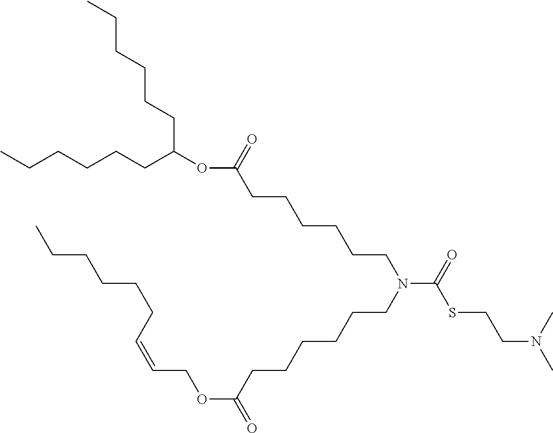 [SMILES: CCCCCC/C=C\COC(=O)CCCCCCN(CCCCCCC(=O)OC(CCCCCC)CCCCCC)C(=O)SCCN(C)C] | ATX-0110 |
| 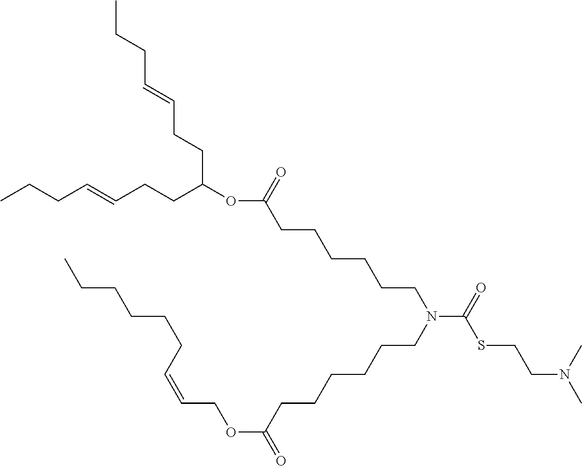 [SMILES: CCC/C=C/CCC(CC/C=C/CCC)OC(=O)CCCCCCN(CCCCCCC(=O)OC/C=C\CCCCCC)C(=O)SCCN(C)C] | ATX-0118 |
| 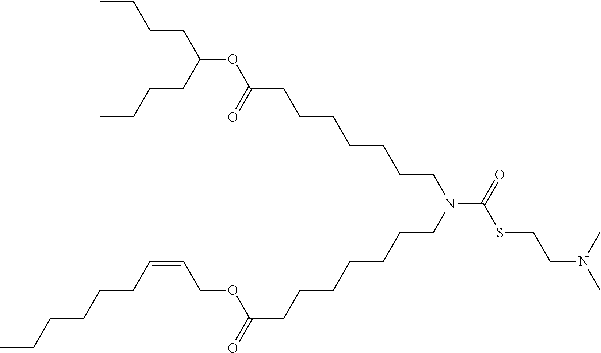 [SMILES: CCCCCC/C=C\COC(=O)CCCCCCCN(CCCCCCCC(=O)OC(CCCC)CCCC)C(=O)SCCN(C)C] | ATX-0108 |
| 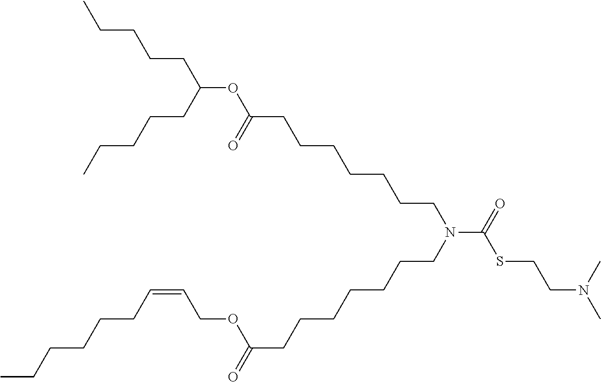 [SMILES: CCCCCC/C=C\COC(=O)CCCCCCCN(CCCCCCCC(=O)OC(CCCCC)CCCCC)C(=O)SCCN(C)C] | ATX-0107 |
| 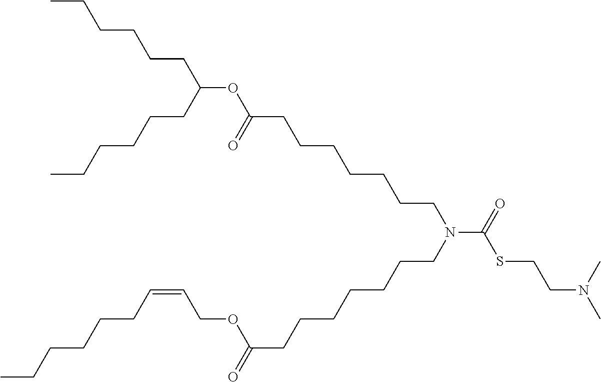 [SMILES: CCCCCC/C=C\COC(=O)CCCCCCCN(CCCCCCCC(=O)OC(CCCCCC)CCCCCC)C(=O)SCCN(C)C] | ATX-093 |
| 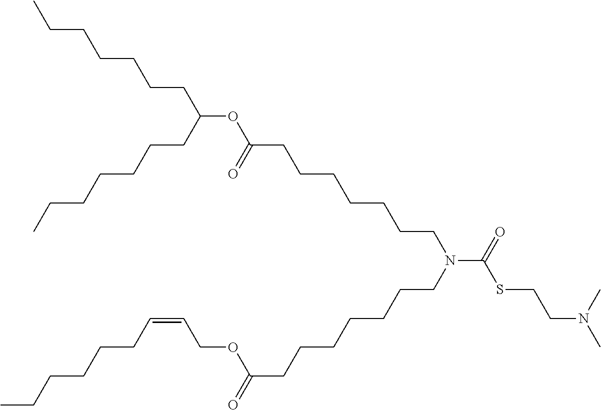 [SMILES: CCCCCC/C=C\COC(=O)CCCCCCCN(CCCCCCCC(=O)OC(CCCCCCC)CCCCCCC)C(=O)SCCN(C)C] | ATX-097 |
| 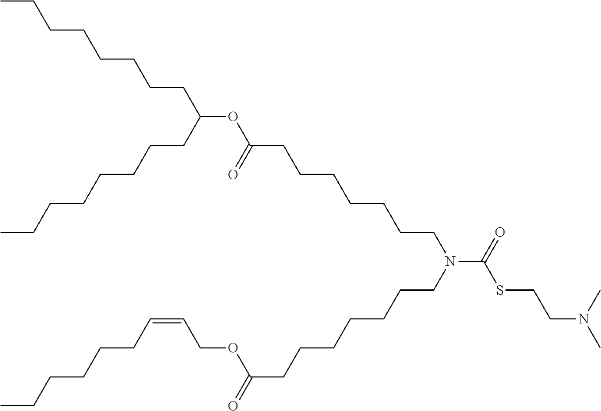 [SMILES: CCCCCC/C=C\COC(=O)CCCCCCCN(CCCCCCCC(=O)OC(CCCCCCCC)CCCCCCCC)C(=O)SCCN(C)C] | ATX-096 |
| 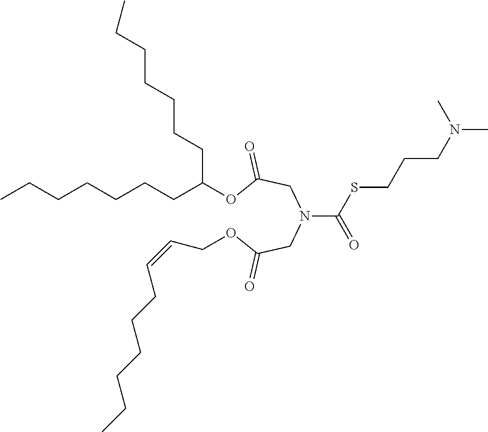 [SMILES: CCCCCC/C=C\COC(=O)CN(CC(=O)OC(CCCCCCC)CCCCCCC)C(=O)SCCCN(C)C] | ATX-0111 |
| 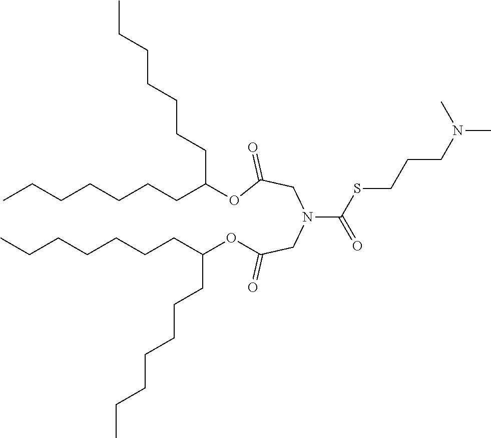 [SMILES: CCCCCCCC(CCCCCCC)OC(=O)CN(CC(=O)OC(CCCCCCC)CCCCCCC)C(=O)SCCCN(C)C] | ATX-0132 |
| 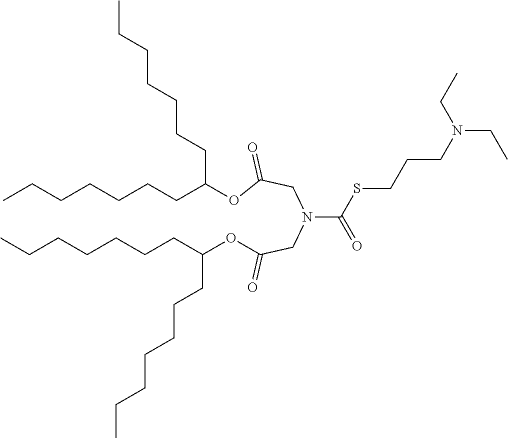 [SMILES: CCCCCCCC(CCCCCCC)OC(=O)CN(CC(=O)OC(CCCCCCC)CCCCCCC)C(=O)SCCCN(CC)CC] | ATX-0134 |
| 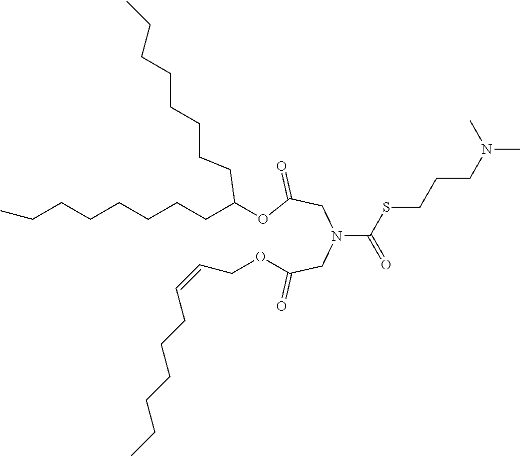 [SMILES: CCCCCC/C=C\COC(=O)CN(CC(=O)OC(CCCCCCCC)CCCCCCCC)C(=O)SCCCN(C)C] | ATX-0100 |
| 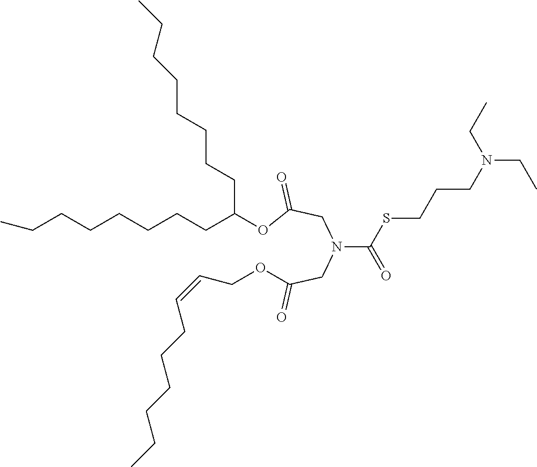 [SMILES: CCCCCC/C=C\COC(=O)CN(CC(=O)OC(CCCCCCCC)CCCCCCCC)C(=O)SCCCN(CC)CC] | ATX-0117 |
| 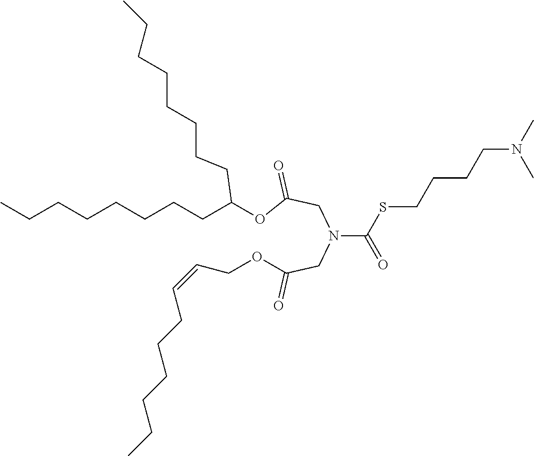 [SMILES: CCCCCC/C=C\COC(=O)CN(CC(=O)OC(CCCCCCCC)CCCCCCCC)C(=O)SCCCCN(C)C] | ATX-0114 |
| 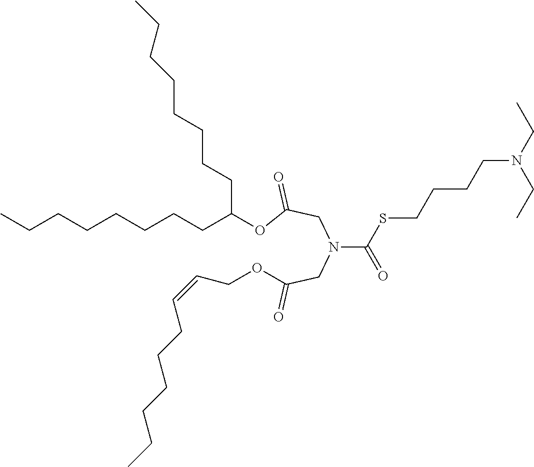 [SMILES: CCCCCC/C=C\COC(=O)CN(CC(=O)OC(CCCCCCCC)CCCCCCCC)C(=O)SCCCCN(CC)CC] | ATX-0115 |
| 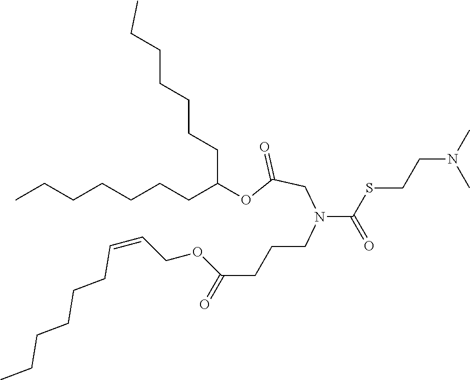 [SMILES: CCCCCC/C=C\COC(=O)CCCN(CC(=O)OC(CCCCCCC)CCCCCCC)C(=O)SCCN(C)C] | ATX-0101 |
| 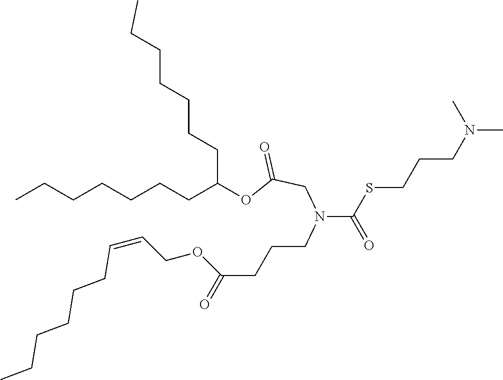 [SMILES: CCCCCC/C=C\COC(=O)CCCN(CC(=O)OC(CCCCCCC)CCCCCCC)C(=O)SCCCN(C)C] | ATX-0106 |
| 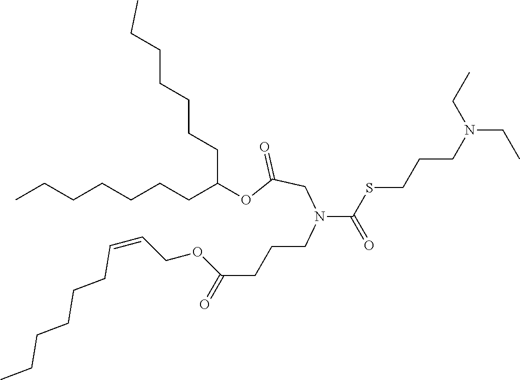 [SMILES: CCCCCC/C=C\COC(=O)CCCN(CC(=O)OC(CCCCCCC)CCCCCCC)C(=O)SCCCN(CC)CC] | ATX-0116 |
| 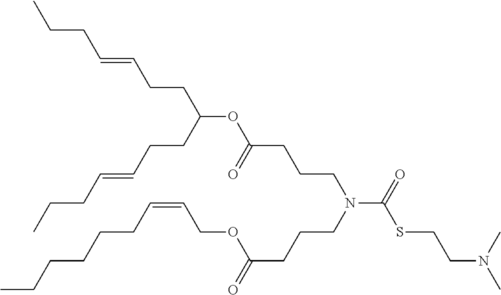 [SMILES: CCC/C=C/CCC(CC/C=C/CCC)OC(=O)CCCN(CCCC(=O)OC/C=C\CCCCCC)C(=O)SCCN(C)C] | ATX-0123 |
| 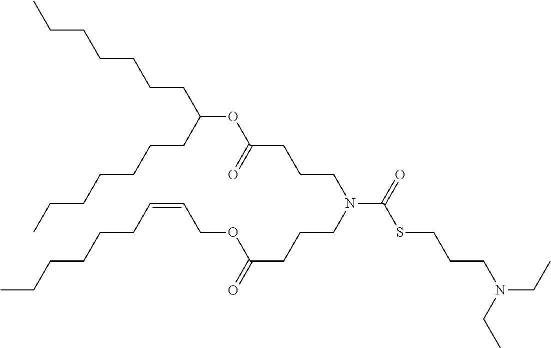 [SMILES: CCCCCC/C=C\COC(=O)CCCN(CCCC(=O)OC(CCCCCCC)CCCCCCC)C(=O)SCCCN(CC)CC] | ATX-0122 |
| 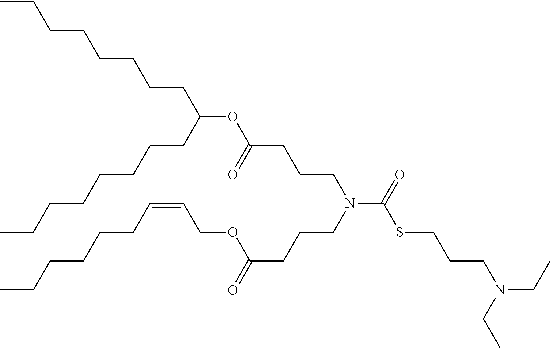 [SMILES: CCCCCC/C=C\COC(=O)CCCN(CCCC(=O)OC(CCCCCCCC)CCCCCCCC)C(=O)SCCCN(CC)CC] | ATX-0124 |
| 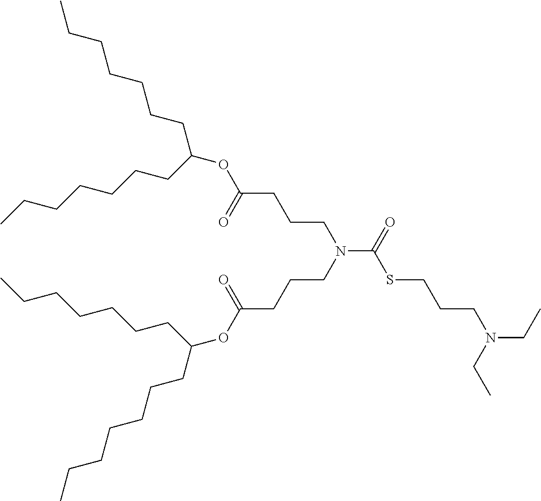 [SMILES: CCCCCCCC(CCCCCCC)OC(=O)CCCN(CCCC(=O)OC(CCCCCCC)CCCCCCC)C(=O)SCCCN(CC)CC] | ATX-0129 |
| 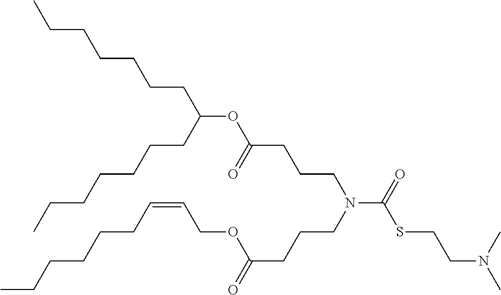 [SMILES: CCCCCC/C=C\COC(=O)CCCN(CCCC(=O)OC(CCCCCCC)CCCCCCC)C(=O)SCCN(C)C] | ATX-081 |
| 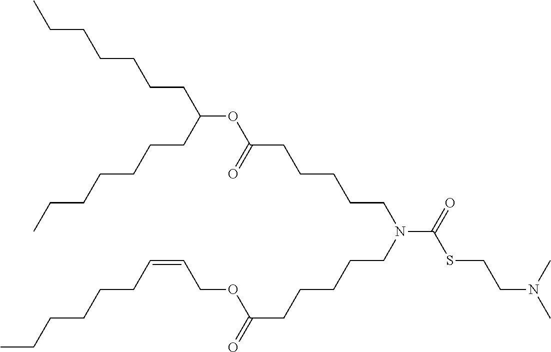 [SMILES: CCCCCC/C=C\COC(=O)CCCCCN(CCCCCC(=O)OC(CCCCCCC)CCCCCCC)C(=O)SCCN(C)C] | ATX-095 |
| 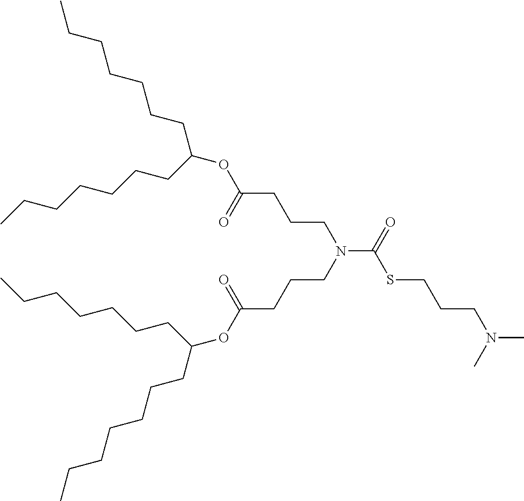 [SMILES: CCCCCCCC(CCCCCCC)OC(=O)CCCN(CCCC(=O)OC(CCCCCCC)CCCCCCC)C(=O)SCCCN(C)C] | ATX-0126 |
| 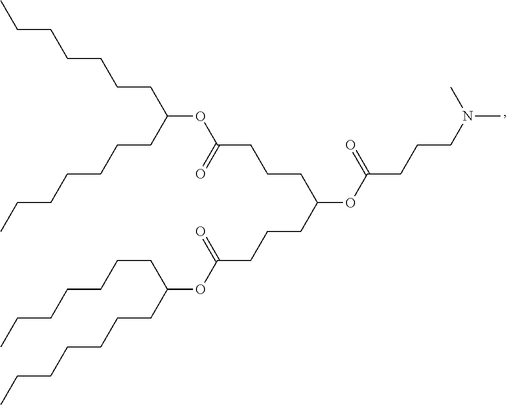 [SMILES: CCCCCCCC(CCCCCCC)OC(=O)CCCC(CCCC(=O)OC(CCCCCCC)CCCCCCC)OC(=O)CCCN(C)C] | ATX-193 |
| 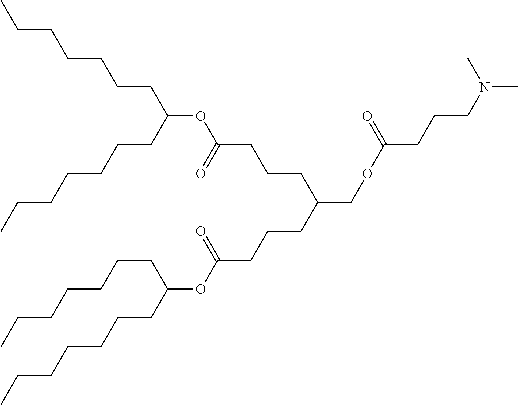 [SMILES: CCCCCCCC(CCCCCCC)OC(=O)CCCC(CCCC(=O)OC(CCCCCCC)CCCCCCC)COC(=O)CCCN(C)C] | ATX-200 |
| 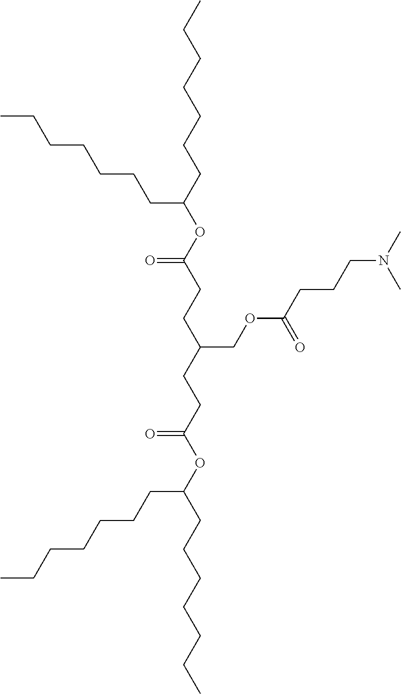 [SMILES: CCCCCCCC(CCCCCCC)OC(=O)CCC(CCC(=O)OC(CCCCCCC)CCCCCCC)COC(=O)CCCN(C)C] | ATX-201 |
| 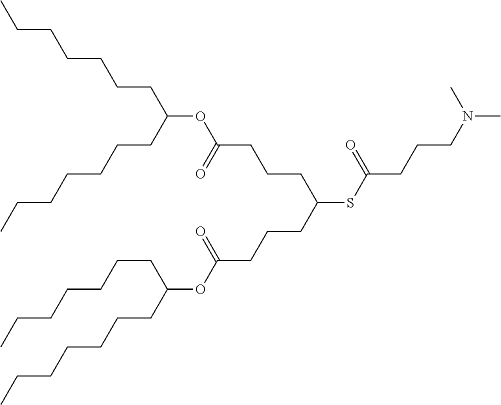 [SMILES: CCCCCCCC(CCCCCCC)OC(=O)CCCC(CCCC(=O)OC(CCCCCCC)CCCCCCC)SC(=O)CCCN(C)C] | ATX-202 |
| 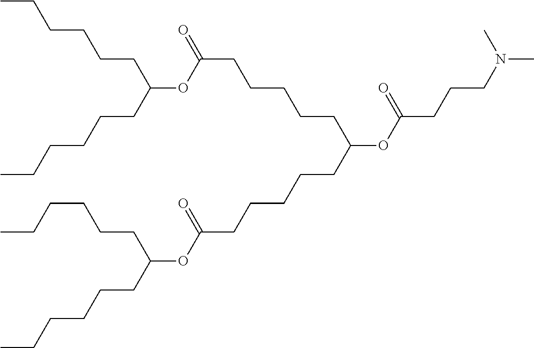 [SMILES: CCCCCCC(CCCCCC)OC(=O)CCCCCC(CCCCCC(=O)OC(CCCCCC)CCCCCC)OC(=O)CCCN(C)C] | ATX-209 |
| 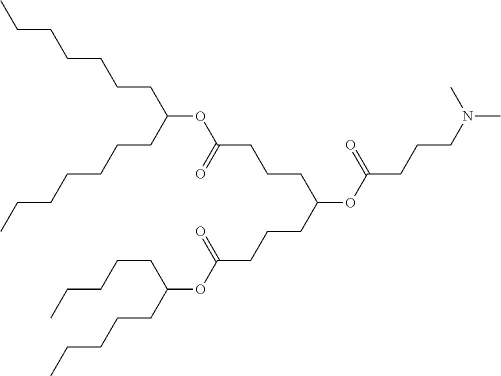 [SMILES: CCCCCCCC(CCCCCCC)OC(=O)CCCC(CCCC(=O)OC(CCCCC)CCCCC)OC(=O)CCCN(C)C] | ATX-210 |
| 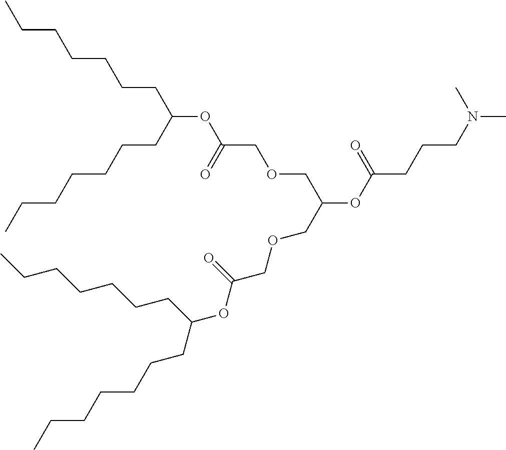 [SMILES: CCCCCCCC(CCCCCCC)OC(=O)COCC(COCC(=O)OC(CCCCCCC)CCCCCCC)OC(=O)CCCN(C)C] | ATX-230 |
| 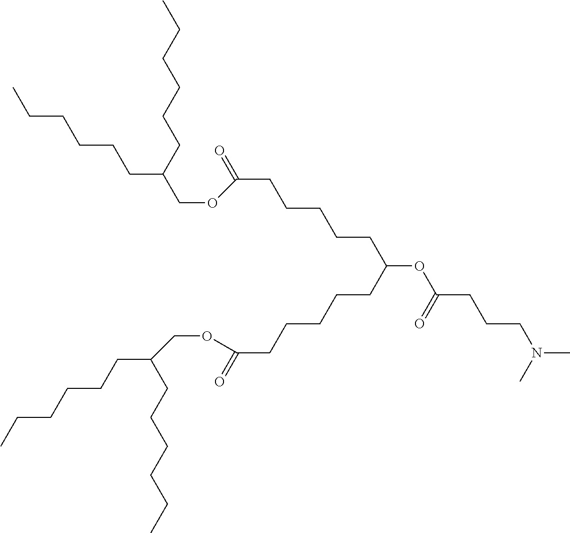 [SMILES: CCCCCCC(CCCCCC)COC(=O)CCCCCC(CCCCCC(=O)OCC(CCCCCC)CCCCCC)OC(=O)CCCN(C)C] | ATX-231 |
| 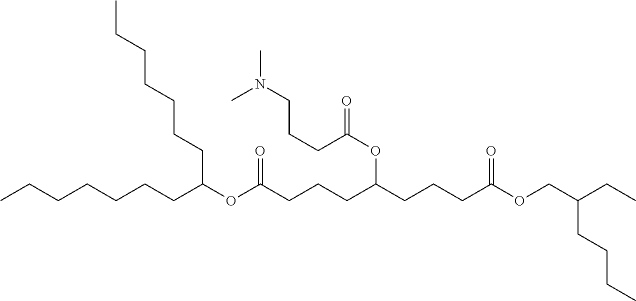 [SMILES: CCCCCCCC(CCCCCCC)OC(=O)CCCC(CCCC(=O)OCC(CC)CCCC)OC(=O)CCCN(C)C] | ATX-232 |
| 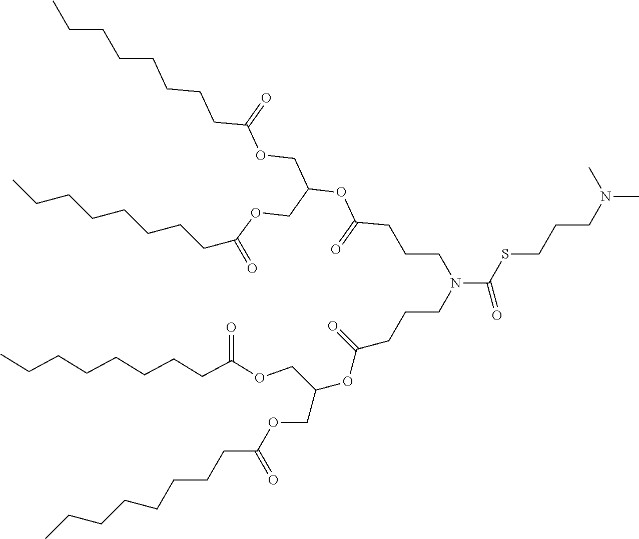 [SMILES: CCCCCCCCC(=O)OCC(COC(=O)CCCCCCCC)OC(=O)CCCN(CCCC(=O)OC(COC(=O)CCCCCCCC)COC(=O)CCCCCCCC)C(=O)SCCCN(C)C] | ATX-194 |
| 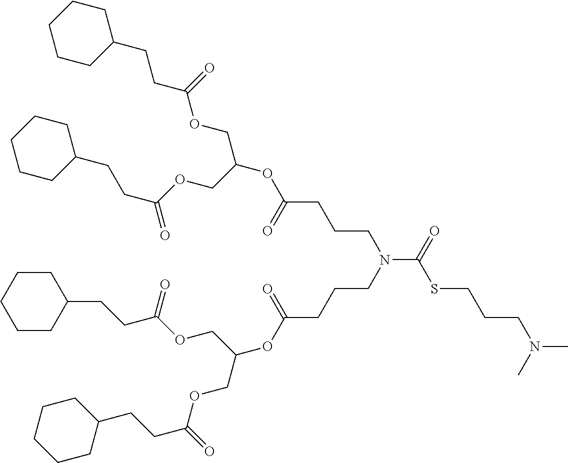 [SMILES: CN(C)CCCSC(=O)N(CCCC(=O)OC(COC(=O)CCC1CCCCC1)COC(=O)CCC1CCCCC1)CCCC(=O)OC(COC(=O)CCC1CCCCC1)COC(=O)CCC1CCCCC1] | ATX-221 |
| 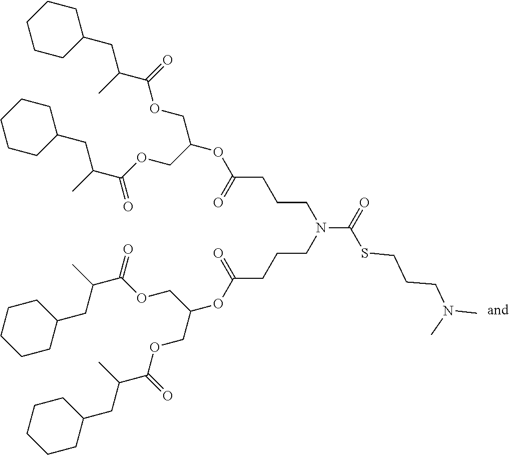 [SMILES: CC(CC1CCCCC1)C(=O)OCC(COC(=O)C(C)CC1CCCCC1)OC(=O)CCCN(CCCC(=O)OC(COC(=O)C(C)CC1CCCCC1)COC(=O)C(C)CC1CCCCC1)C(=O)SCCCN(C)C] | ATX-239 |
| 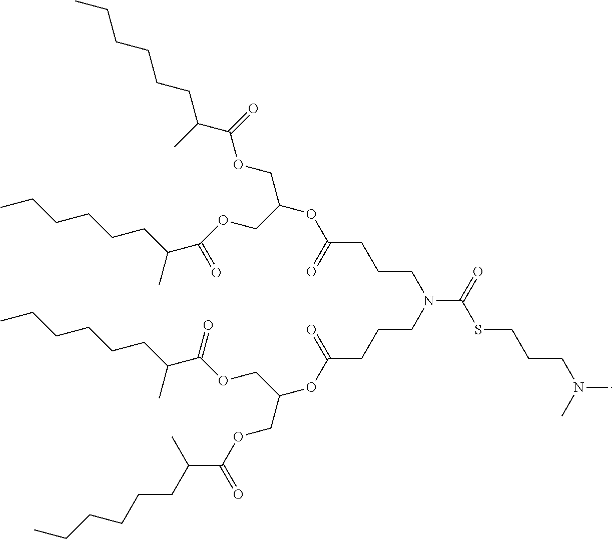 | ATX-240 |

In one aspect, the ionizable cationic lipid of compositions provided herein has a structure of

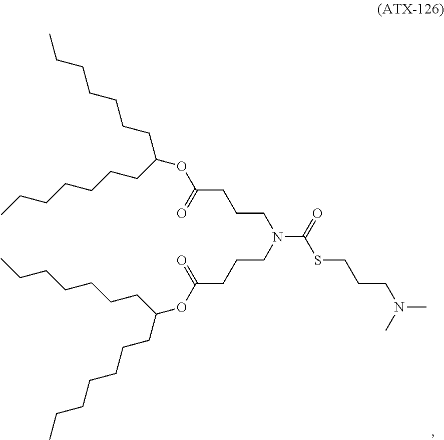 [SMILES: CCCCCCCC(CCCCCCC)OC(=O)CCCN(CCCC(=O)OC(CCCCCCC)CCCCCCC)C(=O)SCCCN(C)C]

or a pharmaceutically acceptable salt thereof.

In another aspect, the ionizable cationic lipid of compositions provided herein has a structure of

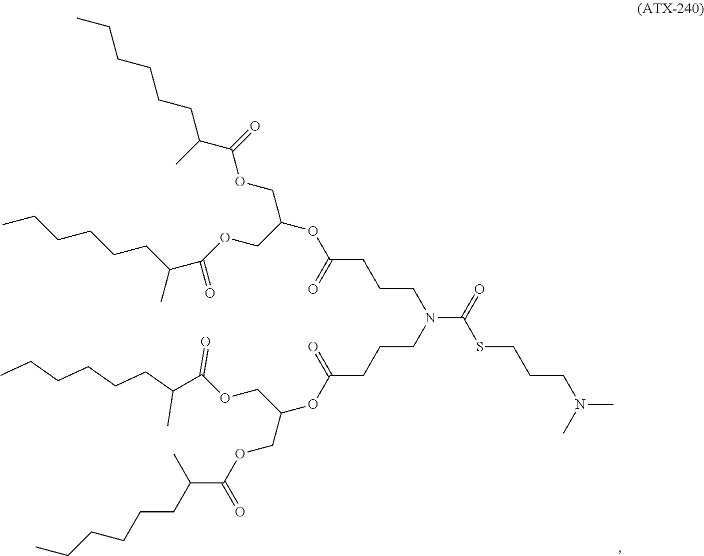 [SMILES: CCCCCCC(C)C(=O)OCC(COC(=O)C(C)CCCCCC)OC(=O)CCCN(CCCC(=O)OC(COC(=O)C(C)CCCCCC)COC(=O)C(C)CCCCCC)C(=O)SCCCN(C)C]

or a pharmaceutically acceptable salt thereof.

### Lipid Formulations/LNPs

Therapies based on the intracellular delivery of nucleic acids to target cells face both extracellular and intracellular barriers. Indeed, naked nucleic acid materials cannot be easily systemically administered due to their toxicity, low stability in serum, rapid renal clearance, reduced uptake by target cells, phagocyte uptake and their ability in activating the immune response, all features that preclude their clinical development. When exogenous nucleic acid material (e.g., mRNA) enters the human biological system, it is recognized by the reticuloendothelial system (RES) as foreign pathogens and cleared from blood circulation before having the chance to encounter target cells within or outside the vascular system. It has been reported that the half-life of naked nucleic acid in the blood stream is around several minutes (Kawabata K, Takakura Y, Hashida M Pharm Res. 1995 June; 12(6):825-30). Chemical modification and a proper delivery method can reduce uptake by the RES and protect nucleic acids from degradation by ubiquitous nucleases, which increase stability and efficacy of nucleic acid-based therapies. In addition, RNAs or DNAs are anionic hydrophilic polymers that are not favorable for uptake by cells, which are also anionic at the surface. The success of nucleic acid-based therapies thus depends largely on the development of vehicles or vectors that can efficiently and effectively deliver genetic material to target cells and obtain sufficient levels of expression in vivo with minimal toxicity.

Moreover, upon internalization into a target cell, nucleic acid delivery vectors are challenged by intracellular barriers, including endosome entrapment, lysosomal degradation, nucleic acid unpacking from vectors, translocation across the nuclear membrane (for DNA), release at the cytoplasm (for RNA), and so on. Successful nucleic acid-based therapy thus depends upon the ability of the vector to deliver the nucleic acids to the target sites inside of the cells in order to obtain sufficient levels of a desired activity such as expression of a gene.

While several gene therapies have been able to successfully utilize a viral delivery vector (e.g., AAV), lipid-based formulations have been increasingly recognized as one of the most promising delivery systems for RNA and other nucleic acid compounds due to their biocompatibility and their ease of large-scale production. One of the most significant advances in lipid-based nucleic acid therapies happened in August 2018 when Patisiran (ALN-TTR02) was the first siRNA therapeutic approved by the Food and Drug Administration (FDA) and by the European Commission (EC). ALN-TR02 is an siRNA formulation based upon the so-called Stable Nucleic Acid Lipid Particle (SNALP) transfecting technology. Despite the success of Patisiran, the delivery of nucleic acid therapeutics, including mRNA, via lipid formulations is still under ongoing development.

Some art-recognized lipid-formulated delivery vehicles for nucleic acid therapeutics include, according to various embodiments, polymer based carriers, such as polyethyleneimine (PEI), lipid nanoparticles and liposomes, nanoliposomes, ceramide-containing nanoliposomes, multivesicular liposomes, proteoliposomes, both natural and synthetically-derived exosomes, natural, synthetic and semi-synthetic lamellar bodies, nanoparticulates, micelles, and emulsions. These lipid formulations can vary in their structure and composition, and as can be expected in a rapidly evolving field, several different terms have been used in the art to describe a single type of delivery vehicle. At the same time, the terms for lipid formulations have varied as to their intended meaning throughout the scientific literature, and this inconsistent use has caused confusion as to the exact meaning of several terms for lipid formulations. Among the several potential lipid formulations, liposomes, cationic liposomes, and lipid nanoparticles are specifically described in detail and defined herein for the purposes of the present disclosure.

In some embodiments, the compositions disclosed herein comprise a nitrogen to phosphate ratio (N:P) of about 3:1 to about 8:1 (e.g., about 5:1 to about 7:1). In some embodiments, the N:P ratio is about 7:1 or less. In some embodiments, the N:P ratio is about 6:1 or less. In some embodiments, the N:P ratio is about 5:1 or less.

In one aspect, the present disclosure provides a composition comprising (i) a polynucleotide having a length of about 5,000 to about 20,000 nucleotides, and (ii) an ionizable cationic lipid, wherein the composition comprises a nitrogen to phosphate ratio (N:P) of about 5:1 to about 7:1. In some embodiments, the nitrogen to phosphate ratio (N:P) is about 7:1. In some embodiments, the polynucleotide is a polynucleotide disclosed herein, which may have a length of at least 5,000 nucleotides (e.g., about 6,000, 7,000, 8,000, 9,000, 10,000, 11,000, 12,000, 13,000, 14,000, 15,000, 16,000, 17,000, 18,000, 19,000, 20,000, or more). In some embodiments, the polynucleotide has a length of about 7,500 nucleotide or more. In some embodiments, the polynucleotide has a length of about 10,000 nucleotide or more. In some embodiments, the polynucleotide has a length of about 15,000 nucleotide or more. In some embodiments, the ionizable cationic lipid is an ionizable cationic lipid disclosed herein (e.g., ATX-126 or ATX-240). In some embodiments, the N:P ratio is about 3:1 to about 8:1 (e.g., about 5:1 to about 7:1). In some embodiments, the N:P ratio is about 7:1 or less. In some embodiments, the N:P ratio is about 6:1 or less. In some embodiments, the N:P ratio is about 5:1 or less.

### Liposomes

Conventional liposomes are vesicles that consist of at least one bilayer and an internal aqueous compartment. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.). They generally present as spherical vesicles and can range in size from 20 nm to a few microns. Liposomal formulations can be prepared as a colloidal dispersion or they can be lyophilized to reduce stability risks and to improve the shelf-life for liposome-based drugs. Methods of preparing liposomal compositions are known in the art and would be within the skill of an ordinary artisan.

Liposomes that have only one bilayer are referred to as being unilamellar, and those having more than one bilayer are referred to as multilamellar. The most common types of liposomes are small unilamellar vesicles (SUV), large unilamellar vesicle (LUV), and multilamellar vesicles (MLV). In contrast to liposomes, lysosomes, micelles, and reversed micelles are composed of monolayers of lipids. Generally, a liposome is thought of as having a single interior compartment, however some formulations can be multivesicular liposomes (MVL), which consist of numerous discontinuous internal aqueous compartments separated by several nonconcentric lipid bilayers.

Liposomes have long been perceived as drug delivery vehicles because of their superior biocompatibility, given that liposomes are basically analogs of biological membranes, and can be prepared from both natural and synthetic phospholipids (Int J Nanomedicine. 2014; 9:1833-1843). In their use as drug delivery vehicles, because a liposome has an aqueous solution core surrounded by a hydrophobic membrane, hydrophilic solutes dissolved in the core cannot readily pass through the bilayer, and hydrophobic compounds will associate with the bilayer. Thus, a liposome can be loaded with hydrophobic and/or hydrophilic molecules. When a liposome is used to carry a nucleic acid such as RNA, the nucleic acid will be contained within the liposomal compartment in an aqueous phase.

### Cationic Liposomes

Liposomes can be composed of cationic, anionic, and/or neutral lipids. As an important subclass of liposomes, cationic liposomes are liposomes that are made in whole or part from positively charged lipids, or more specifically a lipid that comprises both a cationic group and a lipophilic portion. In addition to the general characteristics profiled above for liposomes, the positively charged moieties of cationic lipids used in cationic liposomes provide several advantages and some unique structural features. For example, the lipophilic portion of the cationic lipid is hydrophobic and thus will direct itself away from the aqueous interior of the liposome and associate with other nonpolar and hydrophobic species. Conversely, the cationic moiety will associate with aqueous media and more importantly with polar molecules and species with which it can complex in the aqueous interior of the cationic liposome. For these reasons, cationic liposomes are increasingly being researched for use in gene therapy due to their favorability towards negatively charged nucleic acids via electrostatic interactions, resulting in complexes that offer biocompatibility, low toxicity, and the possibility of the large-scale production required for in vivo clinical applications. Cationic lipids suitable for use in cationic liposomes are listed herein below.

### Lipid Nanoparticles

In contrast to liposomes and cationic liposomes, lipid nanoparticles (LNP) have a structure that includes a single monolayer or bilayer of lipids that encapsulates a compound in a solid phase. Thus, unlike liposomes, lipid nanoparticles do not have an aqueous phase or other liquid phase in its interior, but rather the lipids from the bilayer or monolayer shell are directly complexed to the internal compound thereby encapsulating it in a solid core. Lipid nanoparticles are typically spherical vesicles having a relatively uniform dispersion of shape and size. While sources vary on what size qualifies a lipid particle as being a nanoparticle, there is some overlap in agreement that a lipid nanoparticle can have a diameter in the range of from 10 nm to 1000 nm. However, more commonly they are considered to be smaller than 120 nm or even 100 nm.

For lipid nanoparticle nucleic acid delivery systems, the lipid shell is formulated to include an ionizable cationic lipid which can complex to and associate with the negatively charged backbone of the nucleic acid core. Ionizable cationic lipids with apparent pKa values below about 7 have the benefit of providing a cationic lipid for complexing with the nucleic acid's negatively charged backbone and loading into the lipid nanoparticle at pH values below the pKa of the ionizable lipid where it is positively charged. Then, at physiological pH values, the lipid nanoparticle can adopt a relatively neutral exterior allowing for a significant increase in the circulation half-lives of the particles following i.v. administration. In the context of nucleic acid delivery, lipid nanoparticles offer many advantages over other lipid-based nucleic acid delivery systems including high nucleic acid encapsulation efficiency, potent transfection, improved penetration into tissues to deliver therapeutics, and low levels of cytotoxicity and immunogenicity.

Prior to the development of lipid nanoparticle delivery systems for nucleic acids, cationic lipids were widely studied as synthetic materials for delivery of nucleic acid medicines. In these early efforts, after mixing together at physiological pH, nucleic acids were condensed by cationic lipids to form lipid-nucleic acid complexes known as lipoplexes. However, lipoplexes proved to be unstable and characterized by broad size distributions ranging from the submicron scale to a few microns. Lipoplexes, such as the Lipofectamine® reagent, have found considerable utility for in vitro transfection. However, these first-generation lipoplexes have not proven useful in vivo. The large particle size and positive charge (Imparted by the cationic lipid) result in rapid plasma clearance, hemolytic and other toxicities, as well as immune system activation. In some aspects, nucleic acid molecules provided herein and lipids or lipid formulations provided herein form a lipid nanoparticle (LNP).

In other aspects, nucleic acid molecules provided herein are incorporated into a lipid formulation (i.e., a lipid-based delivery vehicle).

In the context of the present disclosure, a lipid-based delivery vehicle typically serves to transport a desired RNA to a target cell or tissue. The lipid-based delivery vehicle can be any suitable lipid-based delivery vehicle known in the art. In some aspects, the lipid-based delivery vehicle is a liposome, a cationic liposome, or a lipid nanoparticle containing a self-replicating RNA or mRNA of the disclosure. In some aspects, the lipid-based delivery vehicle comprises a nanoparticle or a bilayer of lipid molecules and a self-replicating RNA or mRNA of the disclosure. In some aspects, the lipid bilayer further comprises a neutral lipid or a polymer. In some aspects, the lipid formulation comprises a liquid medium. In some aspects, the formulation further encapsulates a nucleic acid. In some aspects, the lipid formulation further comprises a nucleic acid and a neutral lipid or a polymer. In some aspects, the lipid formulation encapsulates the nucleic acid.

The description provides lipid formulations comprising one or more RNA molecules encapsulated within the lipid formulation. In some aspects, the lipid formulation comprises liposomes. In some aspects, the lipid formulation comprises cationic liposomes. In some aspects, the lipid formulation comprises lipid nanoparticles.

In some aspects, the self-replicating RNA or mRNA is fully encapsulated within the lipid portion of the lipid formulation such that the RNA in the lipid formulation is resistant in aqueous solution to nuclease degradation. In other aspects, the lipid formulations described herein are substantially non-toxic to animals such as humans and other mammals.

The lipid formulations of the disclosure also typically have a total lipid:RNA ratio (mass/mass ratio) of from about 1:1 to about 100:1, from about 1:1 to about 50:1, from about 2:1 to about 45:1, from about 3:1 to about 40:1, from about 5:1 to about 45:1, or from about 10:1 to about 40:1, or from about 15:1 to about 40:1, or from about 20:1 to about 40:1; or from about 25:1 to about 45:1; or from about 30:1 to about 45:1; or from about 32:1 to about 42:1; or from about 34:1 to about 42:1. In some aspects, the total lipid:RNA ratio (mass/mass ratio) is from about 30:1 to about 45:1. The ratio may be any value or subvalue within the recited ranges, including endpoints.

The lipid formulations of the present disclosure typically have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, or about 150 nm, and are substantially non-toxic. The diameter may be any value or subvalue within the recited ranges, including endpoints. In addition, nucleic acids, when present in the lipid nanoparticles of the present disclosure, generally are resistant in aqueous solution to degradation with a nuclease.

In some embodiments, the lipid nanoparticle has a size of less than about 500 nm, less than about 400 nm, less than about 300 nm, less than about 200 nm, less than about 100 nm, or less than about 50 nm. In specific embodiments, the lipid nanoparticle has a size of about 55 nm to about 90 nm.

In some aspects, the lipid formulations comprise a self-replicating RNA or mRNA, a cationic lipid (e.g., one or more cationic lipids or salts thereof described herein), a phospholipid, and a conjugated lipid that inhibits aggregation of the particles (e.g., one or more PEG-lipid conjugates). The lipid formulations can also include cholesterol. In one aspect, the cationic lipid is an ionizable cationic lipid.

In the nucleic acid-lipid formulations, the RNA may be fully encapsulated within the lipid portion of the formulation, thereby protecting the nucleic acid from nuclease degradation. In some aspects, a lipid formulation comprising an RNA is fully encapsulated within the lipid portion of the lipid formulation, thereby protecting the nucleic acid from nuclease degradation. In certain aspects, the RNA in the lipid formulation is not substantially degraded after exposure of the particle to a nuclease at 37° C. for at least 20, 30, 45, or 60 minutes. In certain other aspects, the RNA in the lipid formulation is not substantially degraded after incubation of the formulation in serum at 37° C. for at least 30, 45, or 60 minutes or at least 2, 3, 4, 5, 6, 7, 8, 9, 10, 12, 14, 16, 18, 20, 22, 24, 26, 28, 30, 32, 34, or 36 hours. In some aspects, the RNA is complexed with the lipid portion of the formulation. One of the benefits of the formulations of the present disclosure is that the nucleic acid-lipid compositions are substantially non-toxic to animals such as humans and other mammals.

In the context of nucleic acids, full encapsulation may be determined by performing a membrane-impermeable fluorescent dye exclusion assay, which uses a dye that has enhanced fluorescence when associated with nucleic acid. Encapsulation is determined by adding the dye to a lipid formulation, measuring the resulting fluorescence, and comparing it to the fluorescence observed upon addition of a small amount of nonionic detergent. Detergent-mediated disruption of the lipid layer releases the encapsulated nucleic acid, allowing it to interact with the membrane-impermeable dye. Nucleic acid encapsulation may be calculated as E=(I0−I)/I0, where/and I0 refers to the fluorescence intensities before and after the addition of detergent.

In some aspects, the present disclosure provides a nucleic acid-lipid composition comprising a plurality of nucleic acid-liposomes, nucleic acid-cationic liposomes, or nucleic acid-lipid nanoparticles. In some aspects, the nucleic acid-lipid composition comprises a plurality of RNA-liposomes. In some aspects, the nucleic acid-lipid composition comprises a plurality of RNA-cationic liposomes. In some aspects, the nucleic acid-lipid composition comprises a plurality of RNA-lipid nanoparticles.

In some aspects, the lipid formulations comprise RNA that is fully encapsulated within the lipid portion of the formulation, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% (or any fraction thereof or range therein) of the particles have the RNA encapsulated therein. The amount may be any value or subvalue within the recited ranges, including endpoints. The RNA included in any RNA-lipid composition or RNA-lipid formulation provided herein can be a self-replicating RNA or an mRNA.

Depending on the intended use of the lipid formulation, the proportions of the components can be varied, and the delivery efficiency of a particular formulation can be measured using assays known in the art.

In some aspects, nucleic acid molecules provided herein are lipid formulated. The lipid formulation is preferably selected from, but not limited to, liposomes, cationic liposomes, and lipid nanoparticles. In one aspect, a lipid formulation is a cationic liposome or a lipid nanoparticle (LNP) comprising:
(a) an RNA of the present disclosure,
(b) a cationic lipid,
(c) an aggregation reducing agent (such as polyethylene glycol (PEG) lipid or PEG-modified lipid),
(d) optionally a non-cationic lipid (such as a neutral lipid), and
(e) optionally, a sterol.

In another aspect, the cationic lipid is an ionizable cationic lipid. Any ionizable cationic lipid can be included in lipid formulations, including exemplary cationic lipids provided herein.

In some aspects, compositions that include lipids and/or lipid formulations provided herein include an RNA molecule comprising (A) the sequence of SEQ ID NO:1; (B) the sequence of SEQ ID NO:2; (C) the sequence of SEQ ID NO:3; or (D) the sequence of SEQ ID NO:4. In some aspects, compositions provided herein include lipid nanoparticles (LNPs). In some aspects, compositions provided herein include lyophilized LNPs.

Provided herein, in some embodiments, are lipid nanoparticle compositions comprising a lipid formulation comprising i. about 45 mol % to about 55 mol % of an ionizable cationic lipid having the structure of ATX-126:

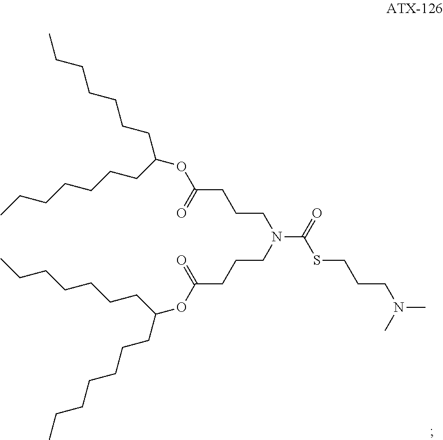 [SMILES: CCCCCCCC(CCCCCCC)OC(=O)CCCN(CCCC(=O)OC(CCCCCCC)CCCCCCC)C(=O)SCCCN(C)C]

ii. about 8 mol % to about 12 mol % DSPC; iii. about 35 mol % to about 42 mol % cholesterol; and iv. about 1.25 mol % to about 1.75 mol % PEG2000-DMG; and b. an RNA molecule having at least 80% identity to the sequence of SEQ ID NO:1, SEQ ID NO:2, SEQ ID NO:3, or SEQ ID NO:4; wherein the lipid formulation encapsulates the RNA molecule and the lipid nanoparticle has a size of about 60 to about 90 nm. In some aspects, lipid nanoparticle compositions provided herein are lyophilized.

Also provided herein, in some embodiments, are lipid nanoparticle compositions comprising a lipid formulation comprising i. about 45 mol % to about 55 mol % of an ionizable cationic lipid having the structure of ATX-240:

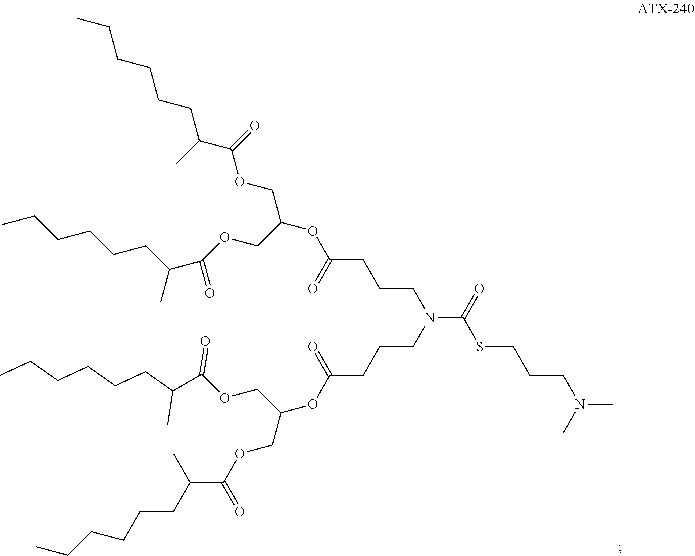 [SMILES: CCCCCCC(C)C(=O)OCC(COC(=O)C(C)CCCCCC)OC(=O)CCCN(CCCC(=O)OC(COC(=O)C(C)CCCCCC)COC(=O)C(C)CCCCCC)C(=O)SCCCN(C)C]

ii. about 8 mol % to about 12 mol % DSPC; iii. about 35 mol % to about 42 mol % cholesterol; and iv. about 1.25 mol % to about 1.75 mol % PEG2000-DMG; and b. an RNA molecule having at least 80% identity to the sequence of SEQ ID NO:1, SEQ ID NO:2, SEQ ID NO:3, or SEQ ID NO:4; wherein the lipid formulation encapsulates the RNA molecule and the lipid nanoparticle has a size of about 60 to about 90 nm. In some aspects, lipid nanoparticle compositions provided herein are lyophilized.

### Cationic Lipids

In one aspect, the lipid nanoparticle formulation comprises (i) at least one cationic lipid; (ii) a helper lipid; (iii) a sterol (e.g., cholesterol); and (iv) a PEG-lipid. In another aspect, the cationic lipid is an ionizable cationic lipid. In yet another aspect, the lipid nanoparticle formulation comprises (i) at least one cationic lipid; (ii) a helper lipid; (iii) a sterol (e.g., cholesterol); and (iv) a PEG-lipid, in a molar ratio of about 40-70% ionizable cationic lipid: about 2-15% helper lipid: about 20-45% sterol; about 0.5-5% PEG-lipid. In a further aspect, the cationic lipid is an ionizable cationic lipid.

In one aspect, the lipid nanoparticle formulation consists of (i) at least one cationic lipid; (ii) a helper lipid; (iii) a sterol (e.g., cholesterol); and (iv) a PEG-lipid. In another aspect, the cationic lipid is an ionizable cationic lipid. In yet another aspect, the lipid nanoparticle formulation consists of (i) at least one cationic lipid; (ii) a helper lipid; (iii) a sterol (e.g., cholesterol); and (iv) a PEG-lipid, in a molar ratio of about 40-70% ionizable cationic lipid: about 2-15% helper lipid: about 20-45% sterol; about 0.5-5% PEG-lipid. In a further aspect, the cationic lipid is an ionizable cationic lipid.

In the presently disclosed lipid formulations, the cationic lipid may be, for example, N,N-dioleyl-N,N-dimethylammonium chloride (DODAC), N,N-distearyl-N,N-dimethylammonium bromide (DDAB), 1,2-dioleoyltrimethylammoniumpropane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanediol (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C₂-DMA), 2,2-dilinoleyl-4-dimethylaminomethyl-[1,3]-dioxolane (DLin-K-DMA), (6Z,9Z,28Z,31Z)-heptatriaconta-6,9,28 31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31 Z)-heptatriaconta-6,9,28,31-tetraen-19-yloxy)-N,N-dimethylbutan-1-amine (MC4 Ether), or any combination thereof. Other cationic lipids include, but are not limited to, N,N-distearyl-N,N-dimethylammonium bromide (DDAB), 3P—(N—(N′,N′-dimethylaminoethane)-carbamoyl)cholesterol (DC-Choi), N-(1-(2,3-dioleyloxy)propyl)-N-2-(sperminecarboxamido)ethyl)-N,N-dimethylammonium trifluoracetate (DOSPA), dioctadecylamidoglycyl carboxyspermine (DOGS), 1,2-dileoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl-[1,3]-dioxolane (XTC). Additionally, commercial preparations of cationic lipids can be used, such as, e.g., LIPOFECTIN (including DOTMA and DOPE, available from GIBCO/BRL), and Lipofectamine (comprising DOSPA and DOPE, available from GIBCO/BRL).

Other suitable cationic lipids are disclosed in International Publication Nos. WO 09/086558, WO 09/127060, WO 10/048536, WO 10/054406, WO 10/088537, WO 10/129709, and WO 2011/153493; U.S. Patent Publication Nos. 2011/0256175, 2012/0128760, and 2012/0027803; U.S. Pat. No. 8,158,601; and Love et al., PNAS, 107(5), 1864-69, 2010, the contents of which are herein incorporated by reference.

The RNA-lipid formulations of the present disclosure can comprise a helper lipid, which can be referred to as a neutral helper lipid, non-cationic lipid, non-cationic helper lipid, anionic lipid, anionic helper lipid, or a neutral lipid. It has been found that lipid formulations, particularly cationic liposomes and lipid nanoparticles have increased cellular uptake if helper lipids are present in the formulation. (Curr. Drug Metab. 2014; 15(9):882-92). For example, some studies have indicated that neutral and zwitterionic lipids such as 1,2-dioleoylsn-glycero-3-phosphatidylcholine (DOPC), Di-Oleoyl-Phosphatidyl-Ethanoalamine (DOPE) and 1,2-DiStearoyl-sn-glycero-3-PhosphoCholine (DSPC), being more fusogenic (i.e., facilitating fusion) than cationic lipids, can affect the polymorphic features of lipid-nucleic acid complexes, promoting the transition from a lamellar to a hexagonal phase, and thus inducing fusion and a disruption of the cellular membrane. (Nanomedicine (Lond). 2014 January; 9(1):105-20). In addition, the use of helper lipids can help to reduce any potential detrimental effects from using many prevalent cationic lipids such as toxicity and immunogenicity.

Non-limiting examples of non-cationic lipids suitable for lipid formulations of the present disclosure include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, palmitoyl, stearoyl, or oleoyl.

Additional examples of non-cationic lipids include sterols such as cholesterol and derivatives thereof. As a helper lipid, cholesterol increases the spacing of the charges of the lipid layer interfacing with the nucleic acid making the charge distribution match that of the nucleic acid more closely. (J. R Soc. Interface. 2012 Mar. 7; 9(68): 548-561). Non-limiting examples of cholesterol derivatives include polar analogues such as 5α-cholestanol, 5α-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5α-cholestanone, and cholesteryl decanoate; and mixtures thereof. In some aspects, the cholesterol derivative is a polar analogue such as cholesteryl-(4′-hydroxy)-butyl ether.

In some aspects, the helper lipid present in the lipid formulation comprises or consists of a mixture of one or more phospholipids and cholesterol or a derivative thereof. In other aspects, the neutral lipid present in the lipid formulation comprises or consists of one or more phospholipids, e.g., a cholesterol-free lipid formulation. In yet other aspects, the neutral lipid present in the lipid formulation comprises or consists of cholesterol or a derivative thereof, e.g., a phospholipid-free lipid formulation.

Other examples of helper lipids include nonphosphorous containing lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, and sphingomyelin.

Other suitable cationic lipids include those having alternative fatty acid groups and other dialkylamino groups, including those, in which the alkyl substituents are different (e.g., N-ethyl-N-methylamino-, and N-propyl-N-ethylamino-). These lipids are part of a subcategory of cationic lipids referred to as amino lipids. In some embodiments of the lipid formulations described herein, the cationic lipid is an amino lipid. In general, amino lipids having less saturated acyl chains are more easily sized, particularly when the complexes must be sized below about 0.3 microns, for purposes of filter sterilization. Amino lipids containing unsaturated fatty acids with carbon chain lengths in the range of C14 to C22 may be used. Other scaffolds can also be used to separate the amino group and the fatty acid or fatty alkyl portion of the amino lipid.

In some embodiments, the lipid formulation comprises the cationic lipid with Formula I according to the patent application US20200163878A1. In this context, the disclosure of US20200163878A1 is also incorporated herein by reference.

In some embodiments, amino or cationic lipids of the present disclosure are ionizable and have at least one protonatable or deprotonatable group, such that the lipid is positively charged at a pH at or below physiological pH (e.g., pH 7.4), and neutral at a second pH, preferably at or above physiological pH. Of course, it will be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwitterionic, are not excluded from use in the disclosure. In certain embodiments, the protonatable lipids have a pKa of the protonatable group in the range of about 4 to about 11. In some embodiments, the ionizable cationic lipid has a pKa of about 5 to about 7. In some embodiments, the pKa of an ionizable cationic lipid is about 6 to about 7.

In some embodiments, the lipid formulation comprises an ionizable cationic lipid of Formula I:

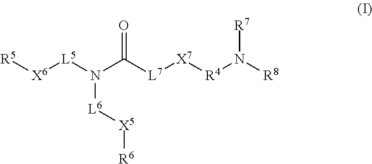

or a pharmaceutically acceptable salt or solvate thereof, wherein R5 and R6 are each independently selected from the group consisting of a linear or branched C1-C31 alkyl, C2-C31 alkenyl or C2-C31 alkynyl and cholesteryl; L5 and L6 are each independently selected from the group consisting of a linear C1-C20 alkyl and C2-C20 alkenyl; X5 is —C(O)O—, whereby —C(O)O—R6 is formed or —OC(O)— whereby —OC(O)—R6 is formed; X6 is —C(O)O— whereby —C(O)O—R5 is formed or —OC(O)— whereby —OC(O)—R5 is formed; X7 is S or O; L7 is absent or lower alkyl; R4 is a linear or branched C₁-C₆ alkyl; and R7 and R8 are each independently selected from the group consisting of a hydrogen and a linear or branched C₁-C₆ alkyl.

In some embodiments, X7 is S.

In some embodiments, X5 is —C(O)O—, whereby —C(O)O—R6 is formed and X6 is —C(O)O— whereby —C(O)O—R5 is formed.

In some embodiments, R7 and R8 are each independently selected from the group consisting of methyl, ethyl and isopropyl.

In some embodiments, L5 and L6 are each independently a C1-C10 alkyl. In some embodiments, L5 is C1-C3 alkyl, and L6 is C1-C5 alkyl. In some embodiments, L6 is C1-C2 alkyl. In some embodiments, L5 and L6 are each a linear C7 alkyl. In some embodiments, L5 and L6 are each a linear C9 alkyl.

In some embodiments, R5 and R6 are each independently an alkenyl. In some embodiments, R6 is alkenyl. In some embodiments, R6 is C2-C9 alkenyl. In some embodiments, the alkenyl comprises a single double bond. In some embodiments, R5 and R6 are each alkyl. In some embodiments, R5 is a branched alkyl. In some embodiments, R5 and R6 are each independently selected from the group consisting of a C9 alkyl, C9 alkenyl and C9 alkynyl. In some embodiments, R5 and R6 are each independently selected from the group consisting of a C11 alkyl, C11 alkenyl and C11 alkynyl. In some embodiments, R5 and R6 are each independently selected from the group consisting of a C7 alkyl, C7 alkenyl and C7 alkynyl. In some embodiments, R5 is —CH((CH2)pCH3)2 or —CH((CH2)pCH3)((CH2)p-1CH3), wherein p is 4-8. In some embodiments, p is 5 and L5 is a C1-C3 alkyl. In some embodiments, p is 6 and L5 is a C3 alkyl. In some embodiments, p is 7. In some embodiments, p is 8 and L5 is a C1-C3 alkyl. In some embodiments, R5 consists of —CH((CH2)pCH3)((CH2)p-1CH3), wherein p is 7 or 8.

In some embodiments, R4 is ethylene or propylene. In some embodiments, R4 is n-propylene or isobutylene.

In some embodiments, L7 is absent, R4 is ethylene, X7 is S and R7 and R8 are each methyl. In some embodiments, L7 is absent, R4 is n-propylene, X7 is S and R7 and R8 are each methyl. In some embodiments, L7 is absent, R4 is ethylene, X7 is S and R7 and R8 are each ethyl.

In some embodiments, X7 is S, X5 is —C(O)O—, whereby —C(O)O—R6 is formed, X6 is —C(O)O— whereby —C(O)O—R5 is formed, L5 and L6 are each independently a linear C3-C7 alkyl, L7 is absent, R5 is —CH((CH2)pCH3)2, and R6 is C7-C12 alkenyl. In some further embodiments, p is 6 and R6 is C9 alkenyl.

In embodiments, any one or more lipids recited herein may be expressly excluded.

In some aspects, the helper lipid comprises from about 2 mol % to about 20 mol %, from about 3 mol % to about 18 mol %, from about 4 mol % to about 16 mol %, about 5 mol % to about 14 mol %, from about 6 mol % to about 12 mol %, from about 5 mol % to about 10 mol %, from about 5 mol % to about 9 mol %, or about 2 mol %, about 3 mol %, about 4 mol %, about 5 mol %, about 6 mol %, about 7 mol %, about 8 mol %, about 9 mol %, about 10 mol %, about 11 mol %, or about 12 mol % (or any fraction thereof or the range therein) of the total lipid present in the lipid formulation.

The lipid portion, or the cholesterol or cholesterol derivative in the lipid formulation may comprise up to about 40 mol %, about 45 mol %, about 50 mol %, about 55 mol %, or about 60 mol % of the total lipid present in the lipid formulation. In some aspects, the cholesterol or cholesterol derivative comprises about 15 mol % to about 45 mol %, about 20 mol % to about 40 mol %, about 25 mol % to about 35 mol %, or about 28 mol % to about 35 mol %; or about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 36 mol %, or about 37 mol % of the total lipid present in the lipid formulation.

In specific embodiments, the lipid portion of the lipid formulation is about 35 mol % to about 42 mol % cholesterol.

In some aspects, the phospholipid component in the mixture may comprise from about 2 mol % to about 20 mol %, from about 3 mol % to about 18 mol %, from about 4 mol % to about 16 mol %, about 5 mol % to about 14 mol %, from about 6 mol % to about 12 mol %, from about 5 mol % to about 10 mol %, from about 5 mol % to about 9 mol %, or about 2 mol %, about 3 mol %, about 4 mol %, about 5 mol %, about 6 mol %, about 7 mol %, about 8 mol %, about 9 mol %, about 10 mol %, about 11 mol %, or about 12 mol % (or any fraction thereof or the range therein) of the total lipid present in the lipid formulation.

In certain embodiments, the lipid portion of the lipid formulation comprises about, but is not necessarily limited to, 40 mol % to about 60 mol % of the ionizable cationic lipid, about 4 mol % to about 16 mol % DSPC, about 30 mol % to about 47 mol % cholesterol, and about 0.5 mol % to about 3 mol % PEG2000-DMG.

In certain embodiments, the lipid portion of the lipid formulation may comprise, but is not necessarily limited to, about 42 mol % to about 58 mol % of the ionizable cationic lipid, about 6 mol % to about 14 mol % DSPC, about 32 mol % to about 44 mol % cholesterol, and about 1 mol % to about 2 mol % PEG2000-DMG.

In certain embodiments, the lipid portion of the lipid formulation may comprise, but is not necessarily limited to, about 45 mol % to about 55 mol % of the ionizable cationic lipid, about 8 mol % to about 12 mol % DSPC, about 35 mol % to about 42 mol % cholesterol, and about 1.25 mol % to about 1.75 mol % PEG2000-DMG.

The percentage of helper lipid present in the lipid formulation is a target amount, and the actual amount of helper lipid present in the formulation may vary, for example, by ±5 mol %.

A lipid formulation that includes a cationic lipid compound or ionizable cationic lipid compound may be on a molar basis about 30-70% cationic lipid compound, about 25-40% cholesterol, about 2-15% helper lipid, and about 0.5-5% of a polyethylene glycol (PEG) lipid, wherein the percent is of the total lipid present in the formulation. In some aspects, the composition is about 40-65% cationic lipid compound, about 25-35% cholesterol, about 3-9% helper lipid, and about 0.5-3% of a PEG-lipid, wherein the percent is of the total lipid present in the formulation.

The formulation may be a lipid particle formulation, for example containing 8-30% nucleic acid compound, 5-30% helper lipid, and 0-20% cholesterol; 4-25% cationic lipid, 4-25% helper lipid, 2-25% cholesterol, 10-35% cholesterol-PEG, and 5% cholesterol-amine; or 2-30% cationic lipid, 2-30% helper lipid, 1-15% cholesterol, 2-35% cholesterol-PEG, and 1-20% cholesterol-amine; or up to 90% cationic lipid and 2-10% helper lipids, or even 100% cationic lipid.

### Lipid Conjugates

The lipid formulations described herein may further comprise a lipid conjugate. The conjugated lipid is useful in that it prevents the aggregation of particles. Suitable conjugated lipids include, but are not limited to, PEG-lipid conjugates, cationic-polymer-lipid conjugates, and mixtures thereof. Furthermore, lipid delivery vehicles can be used for specific targeting by attaching ligands (e.g., antibodies, peptides, and carbohydrates) to its surface or to the terminal end of the attached PEG chains (Front Pharmacol. 2015 Dec. 1; 6:286).

In some aspects, the lipid conjugate is a PEG-lipid. The inclusion of polyethylene glycol (PEG) in a lipid formulation as a coating or surface ligand, a technique referred to as PEGylation, helps to protect nanoparticles from the immune system and their escape from RES uptake (Nanomedicine (Lond). 2011 June; 6(4):715-28). PEGylation has been used to stabilize lipid formulations and their payloads through physical, chemical, and biological mechanisms. Detergent-like PEG lipids (e.g., PEG-DSPE) can enter the lipid formulation to form a hydrated layer and steric barrier on the surface. Based on the degree of PEGylation, the surface layer can be generally divided into two types, brush-like and mushroom-like layers. For PEG-DSPE-stabilized formulations, PEG will take on the mushroom conformation at a low degree of PEGylation (usually less than 5 mol %) and will shift to brush conformation as the content of PEG-DSPE is increased past a certain level (Journal of Nanomaterials. 2011; 2011:12). PEGylation leads to a significant increase in the circulation half-life of lipid formulations (Annu. Rev. Biomed. Eng. 2011 Aug. 15; 130:507-30; J. Control Release. 2010 Aug. 3; 145(3):178-81).

Examples of PEG-lipids include, but are not limited to, PEG coupled to dialkyloxypropyls (PEG-DAA), PEG coupled to diacylglycerol (PEG-DAG), methoxypolyethyleneglycol (PEG-DMG or PEG2000-DMG), PEG coupled to phospholipids such as phosphatidylethanolamine (PEG-PE), PEG conjugated to ceramides, PEG conjugated to cholesterol or a derivative thereof, and mixtures thereof.

PEG is a linear, water-soluble polymer of ethylene PEG repeating units with two terminal hydroxyl groups. PEGs are classified by their molecular weights and include the following: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), monomethoxypolyethylene glycol-imidazolyl-carbonyl (MePEG-IM), as well as such compounds containing a terminal hydroxyl group instead of a terminal methoxy group (e.g., HO-PEG-S, HO-PEG-S-NHS, HO-PEG-NH2).

The PEG moiety of the PEG-lipid conjugates described herein may comprise an average molecular weight ranging from about 550 daltons to about 10,000 daltons. In certain aspects, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons). In some aspects, the PEG moiety has an average molecular weight of about 2,000 daltons or about 750 daltons. The average molecular weight may be any value or subvalue within the recited ranges, including endpoints.

In certain aspects, the PEG can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester-containing linker moieties and ester-containing linker moieties. In one aspect, the linker moiety is a non-ester-containing linker moiety. Exemplary non-ester-containing linker moieties include, but are not limited to, amido (—C(O)NH—), amino (—NR—), carbonyl (—C(O)—), carbamate (—NHC(O)O—), urea (—NHC(O)NH—), disulfide (—S—S—), ether (—O—), succinyl (—(O)CCH2CH2C(O)—), succinamidyl (—NHC(O)CH2CH2C(O)NH—), ether, as well as combinations thereof (such as a linker containing both a carbamate linker moiety and an amido linker moiety). In one aspect, a carbamate linker is used to couple the PEG to the lipid.

In some aspects, an ester-containing linker moiety is used to couple the PEG to the lipid. Exemplary ester-containing linker moieties include, e.g., carbonate (—OC(O)O—), succinoyl, phosphate esters (—O—(O)POH—O—), sulfonate esters, and combinations thereof.

Phosphatidylethanolamines having a variety of acyl chain groups of varying chain lengths and degrees of saturation can be conjugated to PEG to form the lipid conjugate. Such phosphatidylethanolamines are commercially available or can be isolated or synthesized using conventional techniques known to those of skill in the art. Phosphatidylethanolamines containing saturated or unsaturated fatty acids with carbon chain lengths in the range of C₁₀ to C₂₀ are preferred. Phosphatidylethanolamines with mono- or di-unsaturated fatty acids and mixtures of saturated and unsaturated fatty acids can also be used. Suitable phosphatidylethanolamines include, but are not limited to, dimyristoyl-phosphatidylethanolamine (DMPE), dipalmitoyl-phosphatidylethanolamine (DPPE), dioleoyl-phosphatidylethanolamine (DOPE), and distearoyl-phosphatidylethanolamine (DSPE).

In some aspects, the PEG-DAA conjugate is a PEG-didecyloxypropyl (C10) conjugate, a PEG-dilauryloxypropyl (C12) conjugate, a PEG-dimyristyloxypropyl (C14) conjugate, a PEG-dipalmityloxypropyl (C16) conjugate, or a PEG-distearyloxypropyl (C18) conjugate. In some aspects, the PEG has an average molecular weight of about 750 or about 2,000 daltons. In some aspects, the terminal hydroxyl group of the PEG is substituted with a methyl group.

In addition to the foregoing, other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl, methacrylamide, polymethacrylamide, and polydimethylacrylamide, polylactic acid, polyglycolic acid, and derivatized celluloses such as hydroxymethylcellulose or hydroxyethylcellulose.

In some aspects, the lipid conjugate (e.g., PEG-lipid) comprises from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 0.9 mol % to about 1.6 mol %, from about 0.9 mol % to about 1.8 mol %, from about 1 mol % to about 1.8 mol %, from about 1 mol % to about 1.7 mol %, from about 1.2 mol % to about 1.8 mol %, from about 1.2 mol % to about 1.7 mol %, from about 1.3 mol % to about 1.6 mol %, or from about 1.4 mol % to about 1.6 mol % (or any fraction thereof or range therein) of the total lipid present in the lipid formulation. In other embodiments, the lipid conjugate (e.g., PEG-lipid) comprises about 0.5%, 0.6%, 0.7%, 0.8%, 0.9%, 1.0%, 1.2%, 1.3%, 1.4%, 1.5%, 1.6%, 1.7%, 1.8%, 1.9%, 2.0%, 2.5%, 3.0%, 3.5%, 4.0%, 4.5%, or 5%, (or any fraction thereof or range therein) of the total lipid present in the lipid formulation. The amount may be any value or subvalue within the recited ranges, including endpoints.

The percentage of lipid conjugate (e.g., PEG-lipid) present in the lipid formulations of the disclosure is a target amount, and the actual amount of lipid conjugate present in the formulation may vary, for example, by ±0.5 mol %. One of ordinary skill in the art will appreciate that the concentration of the lipid conjugate can be varied depending on the lipid conjugate employed and the rate at which the lipid formulation is to become fusogenic.

In some embodiments, the lipid formulation for any of the compositions described herein comprises a lipoplex, a liposome, a lipid nanoparticle, a polymer-based particle, an exosome, a lamellar body, a micelle, or an emulsion.

### Mechanism of Action for Cellular Uptake of Lipid Formulations

In some aspects, lipid formulations for the intracellular delivery of nucleic acids, particularly liposomes, cationic liposomes, and lipid nanoparticles, are designed for cellular uptake by penetrating target cells through exploitation of the target cells' endocytic mechanisms where the contents of the lipid delivery vehicle are delivered to the cytosol of the target cell. (Nucleic Acid Therapeutics, 28(3):146-157, 2018). Prior to endocytosis, functionalized ligands such as PEG-lipid at the surface of the lipid delivery vehicle are shed from the surface, which triggers internalization into the target cell. During endocytosis, some part of the plasma membrane of the cell surrounds the vector and engulfs it into a vesicle that then pinches off from the cell membrane, enters the cytosol and ultimately enters and moves through the endolysosomal pathway. For ionizable cationic lipid-containing delivery vehicles, the increased acidity as the endosome ages results in a vehicle with a strong positive charge on the surface. Interactions between the delivery vehicle and the endosomal membrane then result in a membrane fusion event that leads to cytosolic delivery of the payload. For RNA payloads, the cell's own internal translation processes will then translate the RNA into the encoded protein. The encoded protein can further undergo postranslational processing, including transportation to a targeted organelle or location within the cell or excretion from the cell.

By controlling the composition and concentration of the lipid conjugate, one can control the rate at which the lipid conjugate exchanges out of the lipid formulation and, in turn, the rate at which the lipid formulation becomes fusogenic. In addition, other variables including, e.g., pH, temperature, or ionic strength, can be used to vary and/or control the rate at which the lipid formulation becomes fusogenic. Other methods which can be used to control the rate at which the lipid formulation becomes fusogenic will become apparent to those of skill in the art upon reading this disclosure. Also, by controlling the composition and concentration of the lipid conjugate, one can control the liposomal or lipid particle size.

### Lipid Formulation Manufacture

There are many different methods for the preparation of lipid formulations comprising a nucleic acid. (Curr. Drug Metabol. 2014, 15, 882-892; Chem. Phys. Lipids 2014, 177, 8-18; Int. J. Pharm. Stud. Res. 2012, 3, 14-20). The techniques of thin film hydration, double emulsion, reverse phase evaporation, microfluidic preparation, dual assymetric centrifugation, ethanol injection, detergent dialysis, spontaneous vesicle formation by ethanol dilution, and encapsulation in preformed liposomes are briefly described herein.

### Thin Film Hydration

In Thin Film Hydration (TFH) or the Bangham method, the lipids are dissolved in an organic solvent, then evaporated through the use of a rotary evaporator leading to a thin lipid layer formation. After the layer hydration by an aqueous buffer solution containing the compound to be loaded, Multilamellar Vesicles (MLVs) are formed, which can be reduced in size to produce Small or Large Unilamellar vesicles (LUV and SUV) by extrusion through membranes or by the sonication of the starting MLV.

### Double Emulsion

Lipid formulations can also be prepared through the Double Emulsion technique, which involves lipids dissolution in a water/organic solvent mixture. The organic solution, containing water droplets, is mixed with an excess of aqueous medium, leading to a water-in-oil-in-water (W/O/W) double emulsion formation. After mechanical vigorous shaking, part of the water droplets collapse, giving Large Unilamellar Vesicles (LUVs).

### Reverse Phase Evaporation

The Reverse Phase Evaporation (REV) method also allows one to achieve LUVs loaded with nucleic acid. In this technique a two-phase system is formed by phospholipids dissolution in organic solvents and aqueous buffer. The resulting suspension is then sonicated briefly until the mixture becomes a clear one-phase dispersion. The lipid formulation is achieved after the organic solvent evaporation under reduced pressure. This technique has been used to encapsulate different large and small hydrophilic molecules including nucleic acids.

### Microfluidic Preparation

The Microfluidic method, unlike other bulk techniques, gives the possibility of controlling the lipid hydration process. The method can be classified in continuous-flow microfluidic and droplet-based microfluidic, according to the way in which the flow is manipulated. In the microfluidic hydrodynamic focusing (MHF) method, which operates in a continuous flow mode, lipids are dissolved in isopropyl alcohol which is hydrodynamically focused in a microchannel cross junction between two aqueous buffer streams. Vesicles size can be controlled by modulating the flow rates, thus controlling the lipids solution/buffer dilution process. The method can be used for producing oligonucleotide (ON) lipid formulations by using a microfluidic device consisting of three-inlet and one-outlet ports.

### Dual Asymmetric Centrifugation

Dual Asymmetric Centrifugation (DAC) differs from more common centrifugation as it uses an additional rotation around its own vertical axis. An efficient homogenization is achieved due to the two overlaying movements generated: the sample is pushed outwards, as in a normal centrifuge, and then it is pushed towards the center of the vial due to the additional rotation. By mixing lipids and an NaCl-solution a viscous vesicular phospholipid gel (VPC) is achieved, which is then diluted to obtain a lipid formulation dispersion. The lipid formulation size can be regulated by optimizing DAC speed, lipid concentration and homogenization time.

### Ethanol Injection

The Ethanol Injection (EI) method can be used for nucleic acid encapsulation. This method provides the rapid injection of an ethanolic solution, in which lipids are dissolved, into an aqueous medium containing nucleic acids to be encapsulated, through the use of a needle. Vesicles are spontaneously formed when the phospholipids are dispersed throughout the medium.

### Detergent Dialysis

The Detergent dialysis method can be used to encapsulate nucleic acids. Briefly lipid and plasmid are solubilized in a detergent solution of appropriate ionic strength, after removing the detergent by dialysis, a stabilized lipid formulation is formed. Unencapsulated nucleic acid is then removed by ion-exchange chromatography and empty vesicles by sucrose density gradient centrifugation. The technique is highly sensitive to the cationic lipid content and to the salt concentration of the dialysis buffer, and the method is also difficult to scale.

### Spontaneous Vesicle Formation by Ethanol Dilution

Stable lipid formulations can also be produced through the Spontaneous Vesicle Formation by Ethanol Dilution method in which a stepwise or dropwise ethanol dilution provides the instantaneous formation of vesicles loaded with nucleic acid by the controlled addition of lipid dissolved in ethanol to a rapidly mixing aqueous buffer containing the nucleic acid.

### Encapsulation in Preformed Liposomes

The entrapment of nucleic acids can also be obtained starting with preformed liposomes through two different methods: (1) A simple mixing of cationic liposomes with nucleic acids which gives electrostatic complexes called “lipoplexes”, where they can be successfully used to transfect cell cultures, but are characterized by their low encapsulation efficiency and poor performance in vivo; and (2) a liposomal destabilization, slowly adding absolute ethanol to a suspension of cationic vesicles up to a concentration of 40% v/v followed by the dropwise addition of nucleic acids achieving loaded vesicles; however, the two main steps characterizing the encapsulation process are too sensitive, and the particles have to be downsized.

### Excipients

The pharmaceutical compositions disclosed herein can be formulated using one or more excipients to: (1) increase stability; (2) increase cell transfection; (3) permit a sustained or delayed release (e.g., from a depot formulation of the polynucleotide, primary construct, or RNA); (4) alter the biodistribution (e.g., target the polynucleotide, primary construct, or RNA to specific tissues or cell types); (5) increase the translation of encoded protein in vivo; and/or (6) alter the release profile of encoded protein in vivo.

The pharmaceutical compositions described herein may be prepared by any method known or hereafter developed in the art of pharmacology. In general, such preparatory methods include the step of associating the active ingredient (i.e., nucleic acid) with an excipient and/or one or more other accessory ingredients. A pharmaceutical composition in accordance with the present disclosure may be prepared, packaged, and/or sold in bulk, as a single unit dose, and/or as a plurality of single unit doses.

Pharmaceutical compositions may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired.

In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, liposomes, lipid nanoparticles, polymers, lipoplexes, core-shell nanoparticles, peptides, proteins, cells transfected with primary DNA construct, or RNA (e.g., for transplantation into a subject), hyaluronidase, nanoparticle mimics and combinations thereof.

Accordingly, the pharmaceutical compositions described herein can include one or more excipients, each in an amount that together increases the stability of the nucleic acid in the lipid formulation, increases cell transfection by the nucleic acid, increases the expression of the encoded protein, and/or alters the release profile of encoded proteins. Further, the RNA of the present disclosure may be formulated using self-assembled nucleic acid nanoparticles.

Various excipients for formulating pharmaceutical compositions and techniques for preparing the composition are known in the art (see Remington: The Science and Practice of Pharmacy, 21st Edition, A. R Gennaro, Lippincott, Williams & Wilkins, Baltimore, Md., 2006; incorporated herein by reference in its entirety). The use of a conventional excipient medium may be contemplated within the scope of the embodiments of the present disclosure, except insofar as any conventional excipient medium may be incompatible with a substance or its derivatives, such as by producing any undesirable biological effect or otherwise interacting in a deleterious manner with any other component(s) of the pharmaceutical composition.

The pharmaceutical compositions of this disclosure may further contain as pharmaceutically acceptable carriers substances as required to approximate physiological conditions, such as pH adjusting and buffering agents, tonicity adjusting agents, and wetting agents, for example, sodium acetate, sodium lactate, sodium chloride, potassium chloride, calcium chloride, sorbitan monolaurate, triethanolamine oleate, and mixtures thereof. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

In certain embodiments of the disclosure, the RNA-lipid formulation may be administered in a time release formulation, for example in a composition which includes a slow-release polymer. The active agent can be prepared with carriers that will protect against rapid release, for example a controlled release vehicle such as a polymer, microencapsulated delivery system, or a bioadhesive gel. Prolonged delivery of the RNA, in various compositions of the disclosure can be brought about by including in the composition agents that delay absorption, for example, aluminum monostearate hydrogels and gelatin.

### Methods of Inducing Immune Responses

Provided herein, in some embodiments, are methods of inducing an immune response in a subject. Any type of immune response can be induced using the methods provided herein, including adaptive and innate immune responses. In one aspect, immune responses induced using the methods provided herein include an antibody response, a cellular immune response, or both an antibody response and a cellular immune response.

Methods of inducing an immune response provided herein include administering to a subject an effective amount of any composition, RNA or DNA molecule, i.e., nucleic acid molecule, provided herein. In one aspect, methods of inducing an immune response include administering to a subject an effective amount of any composition comprising an RNA molecule and a lipid provided herein. In another aspect, methods of inducing an immune response include administering to a subject an effective amount of any pharmaceutical composition comprising an RNA molecule and a lipid formulation provided herein. In some aspects, RNA molecules, compositions, and pharmaceutical composition provided here are vaccines that can elicit a protective or a therapeutic immune response, for example.

As used herein, the term “subject” refers to any individual or patient on which the methods disclosed herein are performed. The term “subject” can be used interchangeably with the term “individual” or “patient.” The subject can be a human, although the subject may be an animal, as will be appreciated by those in the art. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject. As used herein, the term “effective amount” or “therapeutically effective amount” refers to that amount of an RNA molecule, composition, or pharmaceutical composition described herein that is sufficient to effect the intended application, including but not limited to inducing an immune response and/or disease treatment, as defined herein. The therapeutically effective amount may vary depending upon the intended application (e.g., inducing an immune response, treatment, application in vivo), or the subject or patient and disease condition being treated, e.g., the weight and age of the subject, the species, the severity of the disease condition, the manner of administration and the like, which can readily be determined by one of ordinary skill in the art. The term also applies to a dose that will induce a particular response in a target cell. The specific dose will vary depending on the particular RNA molecule, composition, or pharmaceutical composition chosen, the dosing regimen to be followed, whether it is administered in combination with other compounds, timing of administration, the tissue to which it is administered, and the physical delivery system in which it is carried.

Exemplary doses of nucleic molecules that can be administered include about 0.01 μg, about 0.02 μg, about 0.03 μg, about 0.04 μg, about 0.05 μg, about 0.06 μg, about 0.07 μg, about 0.08 μg, about 0.09 μg, about 0.1 μg, about 0.2 μg, about 0.3 μg, about 0.4 μg, about 0.5 μg, about 0.6 μg, about 0.7 μg, about 0.8 μg, about 0.9 μg, about 1.0 μg, about 1.5 μg, about 2.0 μg, about 2.5 μg, about 3.0 μg, about 3.5 μg, about 4.0 μg, about 4.5 μg, about 5.0 μg, about 5.5 μg, about 6.0 μg, about 6.5 μg, about 7.0 μg, about 7.5 μg, about 8.0 μg, about 8.5 μg, about 9.0 μg, about 9.5 μg, about 10 μg, about 11 μg, about 12 μg, about 13μ, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 125 μg, about 150 μg, about 175 μg, about 200 μg, about 250 μg, about 300 μg, about 350 μg, about 400 μg, about 450 μg, about 500 μg, about 600 μg, about 700 μg, about 800 μg, about 900 μg, about 1,000 μg, or more, and any number or range in between. In one aspect, the nucleic acid molecules are RNA molecules. In another aspect, the nucleic acid molecules are DNA molecules. In yet another aspect, nucleic acid molecules include one RNA molecule or one DNA molecule. In a further aspect, nucleic acid molecules include two, three, four, five, six, seven, eight, or more different RNA molecules or DNA molecules. Nucleic acid molecules can have a unit dosage comprising about 0.01 μg to about 1,000 μg or more nucleic acid in a single dose.

In some aspects, compositions provided herein that can be administered include about 0.01 μg, about 0.02 μg, about 0.03 μg, about 0.04 μg, about 0.05 μg, about 0.06 μg, about 0.07 μg, about 0.08 μg, about 0.09 μg, about 0.1 μg, about 0.2 μg, about 0.3 μg, about 0.4 μg, about 0.5 μg, about 0.6 μg, about 0.7 μg, about 0.8 μg, about 0.9 μg, about 1.0 μg, about 1.5 μg, about 2.0 μg, about 2.5 μg, about 3.0 μg, about 3.5 μg, about 4.0 μg, about 4.5 μg, about 5.0 μg, about 5.5 μg, about 6.0 μg, about 6.5 μg, about 7.0 μg, about 7.5 μg, about 8.0 μg, about 8.5 μg, about 9.0 μg, about 9.5 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 125 μg, about 150 μg, about 175 μg, about 200 μg, about 250 μg, about 300 μg, about 350 μg, about 400 μg, about 450 μg, about 500 μg, about 600 μg, about 700 μg, about 800 μg, about 900 μg, about 1,000 μg, or more, and any number or range in between, nucleic acid and lipid. In other aspects, pharmaceutical compositions provided herein that can be administered include about 0.01 μg, about 0.02 μg, about 0.03 μg, about 0.04 μg, about 0.05 μg, about 0.06 μg, about 0.07 μg, about 0.08 μg, about 0.09 μg, about 0.1 μg, about 0.2 μg, about 0.3 μg, about 0.4 μg, about 0.5 μg, about 0.6 μg, about 0.7 μg, about 0.8 μg, about 0.9 μg, about 1.0 μg, about 1.5 μg, about 2.0 μg, about 2.5 μg, about 3.0 μg, about 3.5 μg, about 4.0 μg, about 4.5 μg, about 5.0 μg, about 5.5 μg, about 6.0 μg, about 6.5 μg, about 7.0 μg, about 7.5 μg, about 8.0 μg, about 8.5 μg, about 9.0 μg, about 9.5 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 125 μg, about 150 μg, about 175 μg, about 200 μg, about 250 μg, about 300 μg, about 350 μg, about 400 μg, about 450 μg, about 500 μg, about 600 μg, about 700 μg, about 800 μg, about 900 μg, about 1,000 μg, or more, and any number or range in between, nucleic acid and lipid formulation. In some aspects, compositions or pharmaceutical compositions include one RNA molecule or one DNA molecule. In further aspects, compositions or pharmaceutical compositions include include two, three, four, five, six, seven, eight, or more different RNA molecules or DNA molecules.

In one aspect, compositions provided herein can have a unit dosage comprising about 0.01 μg to about 1,000 μg or more nucleic acid and lipid in a single dose. In another aspect, pharmaceutical compositions provided herein can have a unit dosage comprising about 0.01 μg to about 1,000 μg or more nucleic acid and lipid formulation in a single dose. A vaccine unit dosage can correspond to the unit dosage of nucleic acid molecules, compositions, or pharmaceutical compositions provided herein and that can be administered to a subject. In one aspect, vaccine compositions of the instant disclosure have a unit dosage comprising about 0.01 μg to about 1,000 μg or more nucleic acid and lipid formulation in a single dose. In another aspect, vaccine compositions of the instant disclosure have a unit dosage comprising about 0.01 μg to about 50 μg nucleic acid and lipid formulation in a single dose. In yet another aspect, vaccine compositions of the instant disclosure have a unit dosage comprising about 0.2 μg to about 20 μg nucleic acid and lipid formulation in a single dose.

A dosage form of the composition of this disclosure can be solid, which can be reconstituted in a liquid prior to administration. The solid can be administered as a powder. The solid can be in the form of a capsule, tablet, or gel. In some embodiments, the pharmaceutical composition comprises a nucleic acid lipid formulation that has been lyophilized. In some embodiments, the lyophilized composition may comprise one or more lyoprotectants, such as, including but not necessarily limited to, glucose, trehalose, sucrose, maltose, lactose, mannitol, inositol, hydroxypropyl-β-cyclodextrin, and/or polyethylene glycol. In some embodiments, the lyophilized composition comprises a poloxamer, potassium sorbate, sucrose, or any combination thereof. In specific embodiments, the poloxamer is poloxamer 188. In some embodiments, the lyophilized compositions described herein may comprise about 0.01 to about 1.0% w/w of a poloxamer. In some embodiments, the lyophilized compositions described herein may comprise about 1.0 to about 5.0% w/w of potassium sorbate. The percentages may be any value or subvalue within the recited ranges, including endpoints.

In some embodiments, the lyophilized composition may comprise about 0.01 to about 1.0% w/w of the nucleic acid molecule. In some embodiments, the composition may comprise about 1.0 to about 5.0% w/w lipids. In some embodiments, the composition may comprise about 0.5 to about 2.5% w/w of TRIS buffer. In some embodiments, the composition may comprise about 0.75 to about 2.75% w/w of NaCl. In some embodiments, the composition may comprise about 5 to about 95% w/w of a sugar, about 10 to about 95% w/w of a sugar, about 15 to about 95% w/w of a sugar, about 20 to about 95% w/w of a sugar, about 25 to about 95% w/w of a sugar, about 30 to about 95% w/w of a sugar, about 35 to about 95% w/w of a sugar, about 40 to about 95% w/w of a sugar, about 45 to about 95% w/w of a sugar, about 50 to about 95% w/w of a sugar, about 55 to about 95% w/w of a sugar, about 60 to about 95% w/w of a sugar, about 65 to about 95% w/w of a sugar, about 70 to about 95% w/w of a sugar, about 75 to about 95% w/w of a sugar, about 80 to about 95% w/w of a sugar, or about 85 to about 95% w/w of a sugar. In some embodiments, the composition may comprise about 1 to about 50% w/w of a sugar, about 5 to about 50% w/w of a sugar, about 10 to about 50% w/w of a sugar, about 15 to about 50% w/w of a sugar, about 20 to about 50% w/w of a sugar, about 25 to about 50% w/w of a sugar, about 30 to about 50% w/w of a sugar, about 35 to about 50% w/w of a sugar, about 40 to about 50% w/w of a sugar, or about 45 to about 50% w/w of a sugar. In some embodiments, the composition may comprise about 1 to about 20% w/w of a sugar, about 2 to about 20% w/w of a sugar, about 3 to about 20% w/w of a sugar, about 4 to about 20% w/w of a sugar, about 5 to about 20% w/w of a sugar, about 6 to about 20% w/w of a sugar, about 7 to about 20% w/w of a sugar, about 8 to about 20% w/w of a sugar, about 9 to about 20% w/w of a sugar, about 10 to about 20% w/w of a sugar, about 11 to about 20% w/w of a sugar, about 12 to about 20% w/w of a sugar, about 13 to about 20% w/w of a sugar, about 14 to about 20% w/w of a sugar, about 15 to about 20% w/w of a sugar, about 16 to about 20% w/w of a sugar, about 17 to about 20% w/w of a sugar, about 18 to about 20% w/w of a sugar, or about 19 to about 20% w/w of a sugar. In some embodiments, the composition may comprise about 1 to about 18% w/w of a sugar, about 2 to about 18% w/w of a sugar, about 3 to about 18% w/w of a sugar, about 4 to about 18% w/w of a sugar, about 5 to about 18% w/w of a sugar, about 6 to about 18% w/w of a sugar, about 7 to about 18% w/w of a sugar, about 8 to about 18% w/w of a sugar, about 9 to about 18% w/w of a sugar, about 10 to about 18% w/w of a sugar, about 11 to about 18% w/w of a sugar, about 12 to about 18% w/w of a sugar, about 13 to about 18% w/w of a sugar, about 14 to about 18% w/w of a sugar, about 15 to about 18% w/w of a sugar, about 16 to about 18% w/w of a sugar, or about 17 to about 18% w/w of a sugar. In some embodiments, the composition may comprise about 1 to about 16% w/w of a sugar, about 2 to about 16% w/w of a sugar, about 3 to about 16% w/w of a sugar, about 4 to about 16% w/w of a sugar, about 5 to about 16% w/w of a sugar, about 6 to about 16% w/w of a sugar, about 7 to about 16% w/w of a sugar, about 8 to about 16% w/w of a sugar, about 9 to about 16% w/w of a sugar, about 10 to about 16% w/w of a sugar, about 11 to about 16% w/w of a sugar, about 12 to about 16% w/w of a sugar, about 13 to about 16% w/w of a sugar, about 14 to about 16% w/w of a sugar, or about 15 to about 16% w/w of a sugar. In some embodiments, the composition may comprise about 1 to about 12% w/w of a sugar, about 2 to about 12% w/w of a sugar, about 3 to about 12% w/w of a sugar, about 4 to about 12% w/w of a sugar, about 5 to about 12% w/w of a sugar, about 6 to about 12% w/w of a sugar, about 7 to about 12% w/w of a sugar, about 8 to about 12% w/w of a sugar, about 9 to about 12% w/w of a sugar, about 10 to about 12% w/w of a sugar, or about 11 to about 12% w/w of a sugar. The percentages may be any value or subvalue within the recited ranges, including endpoints. Compositions provided herein can be lyophilized, a liquid, a frozen liquid, or a liquid suspension.

In a preferred embodiment, the dosage form of the pharmaceutical compositions described herein can be a liquid suspension of RNA lipid nanoparticles described herein. In some embodiments, the RNA of RNA lipid nanoparticles is a self-replicating RNA. In some embodiments, the RNA of RNA lipid nanoparticles is an mRNA. In some embodiments, the liquid suspension is in a buffered solution. In some embodiments, the buffered solution comprises a buffer selected from the group consisting of HEPES, MOPS, TES, and TRIS. In some embodiments, the buffer has a pH of about 7.4. In some preferred embodiments, the buffer is HEPES. In some further embodiments, the buffered solution further comprises a cryoprotectant. In some embodiments, the cryoprotectant is selected from a sugar and glycerol or a combination of a sugar and glycerol. In some embodiments, the sugar is a dimeric sugar. In some embodiments, the sugar is sucrose. In some preferred embodiments, the buffer comprises HEPES, sucrose, and glycerol at a pH of 7.4. In certain embodiments, the composition comprises a HEPES, MOPS, TES, or TRIS buffer at a pH of about 7.0 to about 8.5. In some embodiments, the HEPES, MOPS, TES, or TRIS buffer may at a concentration ranging from 7 mg/ml to about 15 mg/ml. The pH or concentration may be any value or subvalue within the recited ranges, including endpoints.

In some embodiments, the suspension is frozen during storage and thawed prior to administration. In some embodiments, the suspension is frozen at a temperature below about 70° C. In some embodiments, the suspension is diluted with sterile water during intravenous administration. In some embodiments, intravenous administration comprises diluting the suspension with about 2 volumes to about 6 volumes of sterile water. In some embodiments, the suspension comprises about 0.1 mg to about 3.0 mg RNA/mL, about 15 mg/mL to about 25 mg/mL of an ionizable cationic lipid, about 0.5 mg/mL to about 2.5 mg/mL of a PEG-lipid, about 1.8 mg/mL to about 3.5 mg/mL of a helper lipid, about 4.5 mg/mL to about 7.5 mg/mL of a cholesterol, about 7 mg/mL to about 15 mg/mL of a buffer, about 2.0 mg/mL to about 4.0 mg/mL of NaCl, about 70 mg/mL to about 110 mg/mL of sucrose, and about 50 mg/mL to about 70 mg/mL of glycerol. In some embodiments, a lyophilized RNA-lipid nanoparticle formulation can be resuspended in a buffer as described herein.

In some embodiments, the compositions of the disclosure are administered to a subject such that a RNA concentration of at least about 0.05 mg/kg, at least about 0.1 mg/kg, at least about 0.5 mg/kg, at least about 1.0 mg/kg, at least about 2.0 mg/kg, at least about 3.0 mg/kg, at least about 4.0 mg/kg, at least about 5.0 mg/kg of body weight is administered in a single dose or as part of single treatment cycle. In some embodiments, the compositions of the disclosure are administered to a subject such that a total amount of at least about 0.1 mg, at least about 0.5 mg, at least about 1.0 mg, at least about 2.0 mg, at least about 3.0 mg, at least about 4.0 mg, at least about 5.0 mg, at least about 6.0 mg, at least about 7.0 mg, at least about 8.0 mg, at least about 9.0 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 105 mg, at least about 110 mg, at least about 115 mg, at least about 120 mg, or at least about 125 mg RNA is administered in one or more doses up to a maximum dose of about 300 mg, about 350 mg, about 400 mg, about 450 mg, or about 500 mg RNA.

Any route of administration can be included in methods provided herein. In some aspects, nucleic acid molecules, i.e., RNA or DNA molecules, compositions, and pharmaceutical compositions provided herein are administered intramuscularly, subcutaneously, intradermally, transdermally, intranasally, orally, sublingually, intravenously, intraperitoneally, topically, by aerosol, or by a pulmonary route, such as by inhalation or by nebulization, for example. In some embodiments, the pharmaceutical compositions described are administered systemically. Suitable routes of administration include, for example, oral, rectal, vaginal, transmucosal, pulmonary including intratracheal or inhaled, or intestinal administration; parenteral delivery, including intradermal, transdermal (topical), intramuscular, subcutaneous, intramedullary injections, as well as intrathecal, direct intraventricular, intravenous, intraperitoneal, or intranasal. In particular embodiments, the intramuscular administration is to a muscle selected from the group consisting of skeletal muscle, smooth muscle and cardiac muscle. In some embodiments, the pharmaceutical composition is administered intravenously.

Pharmaceutical compositions may be administered to any desired tissue. In some embodiments, the RNA delivered is expressed in a tissue different from the tissue in which the lipid formulation or pharmaceutical composition was administered. In preferred embodiments, RNA is delivered and expressed in the liver.

In other aspects, nucleic acid molecules, i.e., RNA or DNA molecules, compositions, and pharmaceutical compositions provided herein are administered intramuscularly.

In some aspects, the subject in which an immune response is induced is a healthy subject. As used herein, the term “healthy subject” refers to a subject not having a condition or disease, including an infectious disease or cancer, for example, or not having a condition or disease against which an immune response is induced. Accordingly, in some aspects, a nucleic acid molecule, composition, or pharmaceutical composition provided herein is administered prophylactically to prevent an infectious disease, for example. A nucleic acid molecule, composition, or pharmaceutical composition provided herein can also be administered therapeutically, i.e., to treat a condition or disease, such as an infection, after the onset of the condition or disease.

As used herein, the terms “treat,” “treatment,” “therapy,” “therapeutic,” and the like refer to obtaining a desired pharmacologic and/or physiologic effect, including, but not limited to, alleviating, delaying or slowing the progression, reducing the effects or symptoms, preventing onset, inhibiting, ameliorating the onset of a diseases or disorder, obtaining a beneficial or desired result with respect to a disease, disorder, or medical condition, such as a therapeutic benefit and/or a prophylactic benefit. “Treatment,” as used herein, includes any treatment of a disease in a mammal, particularly in a human, and includes: (a) preventing the disease from occurring in a subject, including a subject which is predisposed to the disease or at risk of acquiring the disease but has not yet been diagnosed as having it; (b) inhibiting the disease, i.e., arresting its development; and (c) relieving the disease, i.e., causing regression of the disease. A therapeutic benefit includes eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. In some aspects, for prophylactic benefit, treatment or compositions for treatment, including pharmaceutical compositions, are administered to a subject at risk of developing a particular disease, or to a subject reporting one or more of the physiological symptoms of a disease, even though a diagnosis of this disease may not have been made. The methods of the present disclosure may be used with any mammal or other animal. In some aspects, treatment results in a decrease or cessation of symptoms. A prophylactic effect includes delaying or eliminating the appearance of a disease or condition, delaying or eliminating the onset of symptoms of a disease or condition, slowing, halting, or reversing the progression of a disease or condition, or any combination thereof.

Nucleic acid molecules, i.e., RNA or DNA molecules, compositions, and pharmaceutical compositions provided herein can be administered once or multiple times. Accordingly, nucleic acid molecules, compositions, and pharmaceutical compositions provided herein can be administered one, two, three, four, five, six, seven, eight, nine, ten, or more times. Timing between two or more administrations can be one week, two weeks, three weeks, four weeks, five weeks, six weeks, seven weeks, eight weeks, nine weeks, weeks, ten weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or more weeks, and any number or range in between. In some aspects, timing between two or more administrations is one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months, 24 months, or more months, and any number or range in between. In other aspects, timing between two or more administrations can be one year, two years, three years, four years, five years, six years, seven years, eight years, nine years, ten years, or more years, and any number or range in between, Timing between the first and any subsequent administration can be the same or different. In one aspect, nucleic acid molecules, compositions, or pharmaceutical compositions provided herein are administered once.

More than one nucleic acid molecule, composition, or pharmaceutical composition can be administered in the methods provided herein. In one aspect, two or more nucleic acid molecules, compositions, or pharmaceutical compositions provided herein are administered simultaneously. In another aspect, two or more nucleic acid molecules, compositions, or pharmaceutical compositions provided herein are administered sequentially. Simultaneous and sequential administrations can include any number and any combination of nucleic acid molecules, compositions, or pharmaceutical compositions provided herein. Multiple nucleic acid molecules, compositions, or pharmaceutical compositions that are administered together or sequentially can include transgenes encoding different antigenic proteins or fragments thereof. In this manner, immune responses against different antigenic targets can be induced. Two, three, four, five, six, seven, eight, nine, ten, or more nucleic acid molecules, compositions, or pharmaceutical compositions including transgenes encoding different antigenic proteins or fragments thereof can be administered simultaneously or sequentially. Any combination of nucleic acid molecules, compositions, and pharmaceutical compositions including any combination of transgenes can be administered simultaneously or sequentially. In some aspects, administration is simultaneous. In other aspects, administration is sequential. Timing between two or more administrations can be one week, two weeks, three weeks, four weeks, five weeks, six weeks, seven weeks, eight weeks, nine weeks, weeks, ten weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or more weeks, and any number or range in between. In some aspects, timing between two or more administrations is one month, two months, three months, four months, five months, six months, seven months, eight months, nine months, ten months, 11 months, 12 months, 13 months, 14 months, 15 months, months, 16 months, 17 months, 18 months, 19 months, 20 months, 21 months, 22 months, 23 months, 24 months, or more months, and any number or range in between. In other aspects, timing between two or more administrations can be one year, two years, three years, four years, five years, six years, seven years, eight years, nine years, ten years, or more years, and any number or range in between, Timing between the first and any subsequent administration can be the same or different. Nucleic acid molecules, compositions, and pharmaceutical compositions provided herein can be administered with any other vaccine or treatment.

Following administration of the composition to the subject, the protein product encoded by the RNA of the disclosure (e.g., an antigen) is detectable in the target tissues for at least about one to seven days or longer. The amount of protein product necessary to achieve a therapeutic effect will vary depending on antibody titer necessary to generate an immunity to pathogen or disease such as influenza in the patient. For example, the protein product may be detectable in the target tissues at a concentration (e.g., a therapeutic concentration) of at least about 0.025-1.5 μg/ml (e.g., at least about 0.050 μg/ml, at least about 0.075 μg/ml, at least about 0.1 μg/ml, at least about 0.2 μg/ml, at least about 0.3 μg/ml, at least about 0.4 μg/ml, at least about 0.5 μg/ml, at least about 0.6 μg/ml, at least about 0.7 μg/ml, at least about 0.8 μg/ml, at least about 0.9 μg/ml, at least about 1.0 μg/ml, at least about 1.1 μg/ml, at least about 1.2 μg/ml, at least about 1.3 μg/ml, at least about 1.4 μg/ml, or at least about 1.5 μg/ml), for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45 days or longer following administration of the composition to the subject.

In some embodiments, the composition described herein may be administered one time. In some embodiments, the composition described herein may be administered two times.

In some embodiments, the composition may be administered in the form of a booster dose, to a subject who was previously vaccinated against influenza.

In some embodiments, a pharmaceutical composition of the present disclosure is administered to a subject once per month. In some embodiments, a pharmaceutical composition of the present disclosure is administered to a subject twice per month. In some embodiments, a pharmaceutical composition of the present disclosure is administered to a subject three times per month. In some embodiments, a pharmaceutical composition of the present disclosure is administered to a subject four times per month.

Alternatively, the compositions of the present disclosure may be administered in a local rather than systemic manner, for example, via injection of the pharmaceutical composition directly into a targeted tissue, preferably in a depot or sustained release formulation. Local delivery can be affected in various ways, depending on the tissue to be targeted. For example, aerosols containing compositions of the present disclosure can be inhaled (for nasal, tracheal, or bronchial delivery); compositions of the present disclosure can be injected into the site of injury, disease manifestation, or pain, for example; compositions can be provided in lozenges for oral, tracheal, or esophageal application; can be supplied in liquid, tablet or capsule form for administration to the stomach or intestines, can be supplied in suppository form for rectal or vaginal application; or can even be delivered to the eye by use of creams, drops, or even injection. Formulations containing compositions of the present disclosure complexed with therapeutic molecules or ligands can even be surgically administered, for example in association with a polymer or other structure or substance that can allow the compositions to diffuse from the site of implantation to surrounding cells. Alternatively, they can be applied surgically without the use of polymers or supports.

### Combinations

The RNA, such as a self-replicating RNA or mRNA provided herein, formulations thereof, or encoded proteins described herein may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. Preferably, the methods of treatment of the present disclosure encompass the delivery of pharmaceutical, prophylactic, diagnostic, or imaging compositions in combination with agents that may improve their bioavailability, reduce and/or modify their metabolism, inhibit their excretion, and/or modify their distribution within the body. As a non-limiting example, an RNA molecule of the disclosure may be used in combination with a pharmaceutical agent for immunizing or vaccinating a subject. In general, it is expected that agents utilized in combination with the presently disclosed RNA molecules and formulations thereof be utilized at levels that do not exceed the levels at which they are utilized individually. In some embodiments, the levels utilized in combination will be lower than those utilized individually. In one embodiment, the combinations, each or together may be administered according to the split dosing regimens as are known in the art.

### Ranges

Throughout this disclosure, various aspects can be presented in range format. It should be understood that any description in range format is merely for convenience and brevity and not meant to be limiting. Accordingly, the description of a range should be considered to have specifically disclosed all possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example 1, 2, 2.1, 2.2, 2.5, 3, 4, 4.75, 4.8, 4.85, 4.95, 5, 5.5, 5.75, 5.9, 5.00, and 6. This applies to a range of any breadth.

### Illustrative Embodiments

The present disclosure provides the following illustrative embodiments:

Embodiment 1. A composition comprising one or more RNA molecules, wherein the one or more RNA molecules collectively encode a hemagglutinin (HA) polypeptide and a neuraminidase (NA) polypeptide of each of four different strains of influenza virus.

Embodiment 2. The composition of Embodiment 1, wherein for each of the four different strains of influenza virus, the HA polypeptide and NA polypeptide are encoded by the same RNA molecule.

Embodiment 3. The composition of Embodiment 1, wherein the HA and NA polypeptides of a first strain of influenza virus are encoded by a first RNA molecule, the HA and NA polypeptides of a second strain of influenza virus are encoded by a second RNA molecule, the HA and NA polypeptides of a third strain of influenza virus are encoded by a third RNA molecule, and the HA and NA polypeptides of a fourth strain of influenza virus are encoded by a fourth RNA molecule.

Embodiment 4. The composition of Embodiment 3, wherein the first, second, third, and fourth RNA molecules are present in an equimolar ratio.

Embodiment 5. The composition of any one of Embodiments 1-4, wherein each of the one or more RNA molecules further encodes one or more viral replication proteins.

Embodiment 6. The composition of Embodiment 5, wherein the one or more viral replication proteins are alphavirus proteins.

Embodiment 7. The composition of Embodiment 6, wherein each of the one or more RNA molecules encodes in 5′ to 3′ order: (i) the one or more viral replication proteins, (ii) one of the NA polypeptides; and (iii) one of the HA polypeptides.

Embodiment 8. The composition of Embodiment 6 or 7, wherein the alphavirus proteins are from Venezuelan Equine Encephalitis Virus (VEEV).

Embodiment 9. The composition of any one of Embodiments 6-8, wherein the one or more viral replication proteins comprise an alphavirus nonstructural protein 1 (nsP1), an alphavirus nonstructural protein 2 (nsP2), an alphavirus nonstructural protein 3 (nsP3), an alphavirus nonstructural protein 4 (nsP4), or any combination thereof.

Embodiment 10. The composition of Embodiment 9, wherein the one or more viral replication proteins comprises a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, or 100% identity to the RNA sequence encoded by SEQ ID NO: 13.

Embodiment 11. The composition of any one of Embodiments 5-10, wherein sequences encoding the HA and NA polypeptides of at least one of the one or more RNA molecules are preceded by a subgenomic promoter (sgP).

Embodiment 12. The composition of any one of Embodiments 1-11, wherein each HA polypeptide comprises an antigenic fragment of a respective HA protein.

Embodiment 13. The composition of any one of Embodiments 1-12, wherein each NA polypeptide comprises an antigenic fragment of a respective NA protein.

Embodiment 14. The composition of any one of Embodiments 1-13, wherein the four different strains of influenza virus comprise one or more of H1N1, H3N2, Victoria-B, or Yamagata-B.

Embodiment 15. The composition of Embodiment 14, wherein the four different strains of influenza virus comprise Victoria B/Austria/1359417/2021, H3N2 A/Darwin/6/2021, H1N1 A/Wisconsin/588/2019, and Yamagata B/PHUKET/3073/2013.

Embodiment 16. The composition of any one of Embodiments 1-15, wherein each of the one or more RNA molecules further comprise a 5′ untranslated region (UTR).

Embodiment 17. The composition of Embodiment 16, wherein at least one 5′ UTR comprises a viral 5′ UTR, a non-viral 5′ UTR, or a combination of viral and non-viral 5′ UTR sequences.

Embodiment 18. The composition of Embodiment 17, wherein the at least one 5′ UTR comprises an alphavirus 5′ UTR.

Embodiment 19. The composition of Embodiment 18, wherein the alphavirus 5′ UTR comprises a Venezuelan Equine Encephalitis Virus (VEEV) 5′ UTR sequence.

Embodiment 20. The composition of Embodiment 16, wherein at least one 5′ UTR comprises the RNA sequence encoded by SEQ ID NO: 14.

Embodiment 21. The composition of any one of Embodiments 1-20, wherein each of the one or more RNA molecules further comprise a 3′ untranslated region (UTR).

Embodiment 22. The composition of Embodiment 21, wherein at least one 3′ UTR comprises a viral 3′ UTR, a non-viral 3′ UTR, or a combination of viral and non-viral 3′ UTR sequences.

Embodiment 23. The composition of Embodiment 22, wherein the at least one 3′ UTR comprises an alphavirus 3′ UTR sequence.

Embodiment 24. The composition of Embodiment 23, wherein the alphavirus 3′ UTR comprises a Venezuelan Equine Encephalitis Virus (VEEV) 3′ UTR sequence.

Embodiment 25. The composition of Embodiment 21, wherein at least one 3′ UTR comprises the RNA sequence encoded by SEQ ID NO: 15.

Embodiment 26. The composition of any one of Embodiments 1-25, wherein the RNA molecule further comprises a poly-A tail.

Embodiment 27. The composition of any one of Embodiments 1-26, wherein the one or more RNA molecules are self-replicating RNA molecules.

Embodiment 28. The composition of Embodiment 27, wherein the one or more RNA molecules comprise a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, or 100% identity to the RNA sequence encoded by any of SEQ ID NOs:1-4.

Embodiment 29. A composition comprising one or more DNA molecules encoding the one or more RNA molecules of the composition of any one of Embodiments 1-28.

Embodiment 30. The composition of Embodiment 29, wherein each of the one or more DNA molecules comprises a promoter.

Embodiment 31. The composition of Embodiment 30, wherein the promoter of each of the one or more DNA molecules is located 5′ of a 5′ UTR.

Embodiment 32. The composition of Embodiment 31, wherein the promoter is a 7 promoter.

Embodiment 33. A composition comprising an RNA molecule comprising a sequence with at least 80% sequence identity to the RNA sequence encoded by SEQ ID NO: 5.

Embodiment 34. The composition of Embodiment 33, further comprising a sequence with at least 80% sequence identity to the RNA sequence encoded by SEQ ID NO: 9.

Embodiment 35. A composition comprising an RNA molecule comprising a sequence with at least 80% sequence identity to the RNA sequence encoded by SEQ ID NO: 6.

Embodiment 36. The composition of Embodiment 35, further comprising a sequence with at least 80% sequence identity to the RNA sequence encoded by SEQ ID NO: 10.

Embodiment 37. A composition comprising an RNA molecule comprising a sequence with at least 80% sequence identity to the RNA sequence encoded by SEQ ID NO: 7.

Embodiment 38. The composition of Embodiment 37, further comprising a sequence with at least 80% sequence identity to the RNA sequence encoded by SEQ ID NO: 11.

Embodiment 39. A composition comprising an RNA molecule comprising a sequence with at least 80% sequence identity to the RNA sequence encoded by SEQ ID NO: 8.

Embodiment 40. The composition of Embodiment 39, further comprising a sequence with at least 80% sequence identity to the RNA sequence encoded by SEQ ID NO: 12.

Embodiment 41. A composition comprising an RNA molecule comprising a sequence with at least 80% sequence identity to the RNA sequence encoded by SEQ ID NO: 9.

Embodiment 42. A composition comprising an RNA molecule comprising a sequence with at least 80% sequence identity to the RNA sequence encoded by SEQ ID NO: 10.

Embodiment 43. A composition comprising an RNA molecule comprising a sequence with at least 80% sequence identity to the RNA sequence encoded by SEQ ID NO: 11.

Embodiment 44. A composition comprising an RNA molecule comprising a sequence with at least 80% sequence identity to the RNA sequence encoded by SEQ ID NO: 12.

Embodiment 45. A composition comprising an RNA molecule comprising a sequence with at least 80% sequence identity to the RNA sequence encoded by any of SEQ ID NOs: 1-4.

Embodiment 46. The composition of any one of Embodiments 1-45, further comprising an ionizable cationic lipid.

Embodiment 47. The composition of Embodiment 46, wherein the ionizable cationic lipid has a structure of Formula I:

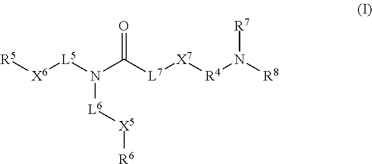

or a pharmaceutically acceptable salt or solvate thereof, wherein R¹ and R⁶ are each independently selected from the group consisting of a linear or branched C₁-C₃₁ alkyl, C₂-C₃₁ alkenyl or C₂-C₃₁ alkynyl and cholesteryl; L⁵ and L⁶ are each independently selected from the group consisting of a linear C₁-C₂₀ alkyl and C₂-C₂₀ alkenyl; X⁵ is —C(O)O—, whereby —C(O)O—R⁶ is formed or —OC(O)— whereby —OC(O)—R⁶ is formed; X⁶ is —C(O)O— whereby —C(O)O—R¹ is formed or —OC(O)— whereby —OC(O)—R¹ is formed; X7 is S or O; L⁷ is absent or lower alkyl; R⁴ is a linear or branched C₁-C₆ alkyl; and R⁷ and R⁸ are each independently selected from the group consisting of a hydrogen and a linear or branched C₁-C₆ alkyl.

Embodiment 48. The composition of Embodiment 46, wherein the ionizable cationic lipid is selected from Table 6.

Embodiment 49. The composition of Embodiment 46, wherein the ionizable cationic lipid is ATX-126:

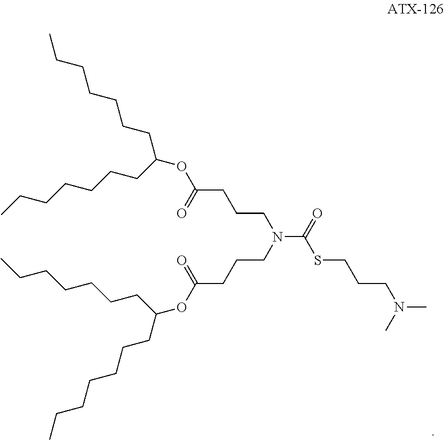 [SMILES: CCCCCCCC(CCCCCCC)OC(=O)CCCN(CCCC(=O)OC(CCCCCCC)CCCCCCC)C(=O)SCCCN(C)C]

Embodiment 50. The composition of Embodiment 46, wherein the ionizable cationic lipid is ATX-240

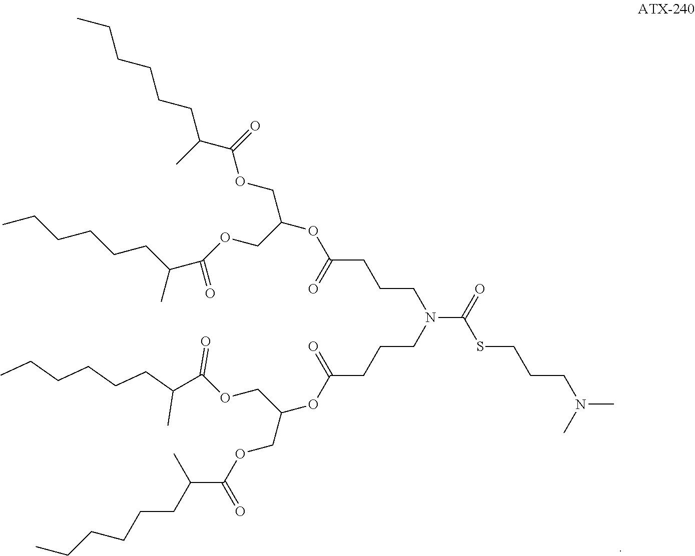 [SMILES: CCCCCCC(C)C(=O)OCC(COC(=O)C(C)CCCCCC)OC(=O)CCCN(CCCC(=O)OC(COC(=O)C(C)CCCCCC)COC(=O)C(C)CCCCCC)C(=O)SCCCN(C)C]

Embodiment 51. The composition of any one of Embodiments 46-50, wherein the composition comprises a nitrogen to phosphate ratio (N:P) of about 5:1 to about 7:1.

Embodiment 52. A method of vaccinating a subject against influenza, the method comprising administering to the subject a composition of any one of Embodiments 1-51.

Embodiment 53. Use of the composition of any one of Embodiments 1-51 in the preparation of a medicament for vaccinating a subject against influenza.

Embodiment 54. A composition comprising (i) a polynucleotide having a length of about 5,000 to about 20,000 nucleotides, and (ii) an ionizable cationic lipid, wherein the composition comprises a nitrogen to phosphate ratio (N:P) of about 5:1 to about 7:1.

Embodiment 55. The composition of Embodiment 54, wherein the nitrogen to phosphate ratio (N:P) is about 7:1.

### EXAMPLES

### Example 1

### Vaccine Design and Construction

Four separate RNAs were designed for use in a quadivalent vaccine, where each RNA encoded for a hemagglutinin (HA) and a neuraminidase (NA) from the same strain of influenza on a single self-amplifying RNA (saRNA). Each antigen was preceded by subgenomic promoter (sgP) sequences from Venezuelan equine encephalitis virus (VEEV) to facilitate replication of the entire saRNA and drive transcription of the HA and NA when delivered into a host cell. The saRNAs also included a poly-A tail of about 130 nucleotides in length.

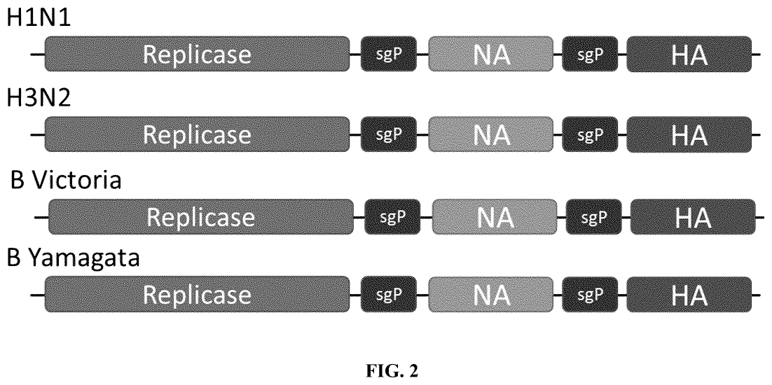

FIG. 2 shows schematics for a vaccine combining four distinct saRNA molecules, where each saRNA molecule encodes in a 5′ to 3′ order: (i) viral replication proteins (replicase portion), (ii) an NA polypeptide, and (iii) an HA polypeptide. Each polypeptide is preceded by a subgenomic promoter, and the NA and HA polypeptides are from the same strain of influenza (H1N1, H3N2, B Victoria, or B Yamagata, as illustrated). saRNAs as illustrated in FIG. 2 may be encoded by a DNA construct and prepared by in vitro transcription from a T7 promoter, for example. saRNAs generally include a poly-A tail at the 3′ end (not shown), which can be encoded by the DNA construct. Constructs and saRNAs encoding both an NA polypeptide and an HA polypeptide are referred to herein as “bicistronic.”

### Example 2

### Immunogenicity

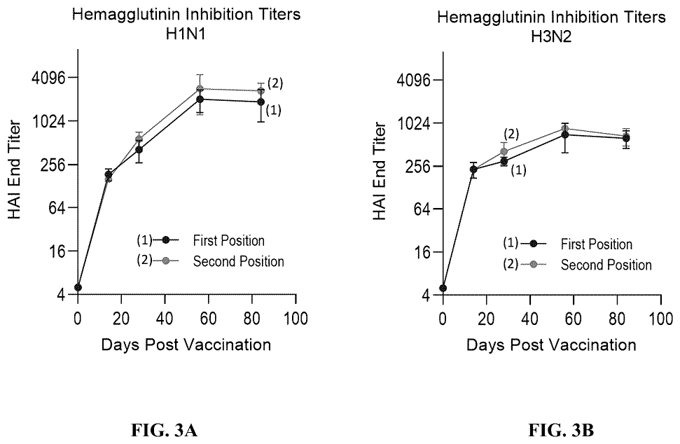

Immunogenicity of a bicistronic design was tested using two hemagglutinin antigens, each at either the 5′, or ‘first’, position or at the 3′, or ‘second’, position. The saRNA constructs encoded hemagglutinins from an H1 strain and an H3 strain on a single saRNA, but in different orders. Female BALB/c mice were vaccined with a single dose of one of the saRNA constructs, and post vaccination HA-inhibition titers for each virus were measured (n=5/group). Serum was taken at the indicated time points (0-100 days post-vaccination) and assessed for hemagglutinin inhibition end titer against HA of either H1N1 or H3N2 influenza. When H1 is encoded in the “first” position, H1N1 HA inhibition titers are lower than those constructs where the H1 is encoded in the second position (FIG. 3A). Similarly, when H3 is encoded in the “first” position, lower H3N2 HA inhibition titers are observed compared to when H3 is encoded in the “second” position (FIG. 3B).

In sum, the data from the study showed that when the HA antigen is located at the “second” position in a bicistronic vector, greater immunogenicity is obtained than when it is located at the “first” position.

### Example 3

### Codon Optimized Sequences

This example describes immunogenicity results for constructs with antigenic sequences optimized using two different codon optimizations, one designated “high codon adaption index” (hCAI) and the other designated “CODEX.” To evaluate immugenicity differences, conventional mRNA vaccines encoding hemagglutinin from H3N2 A/Cambodia/e0826360/2020 strain of influenza were prepared. The only difference between the two vaccine formulations was the sequence encoding the HA antigen (the HA polypeptide sequence encoded by the respective codon optimized sequences was the same).

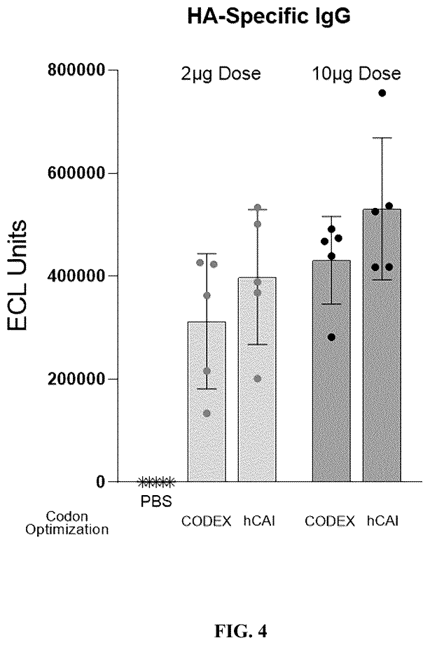

Female BALB/c mice were vaccinated with either the hCAI-based RNA vaccine, or the CODEX-based RNA vaccine, each at either 2 μg or 10 μg doses. HA-specific IgG was assessed by Meso Scale Discovery (MSD) ELISA-based assay with serum taken 28 days post vaccination. PBS was used for control. Results are shown in FIG. 4. Constructs with hCAI-based codon optimization achieved higher HA-specific responses than those with CODEX-based optimization.

Based on these results, hCAI codon optimization was used to produce the HA- and NA-encoding sequences of SEQ ID NOs: 1-4.

### Example 4

Monovalent Vs. Quadrivalent Vaccine Designs

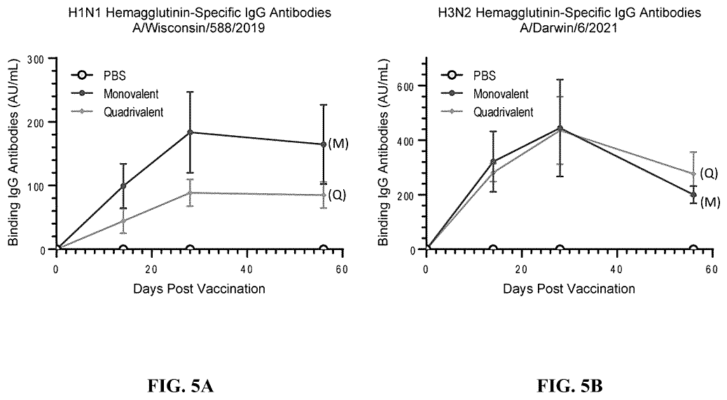

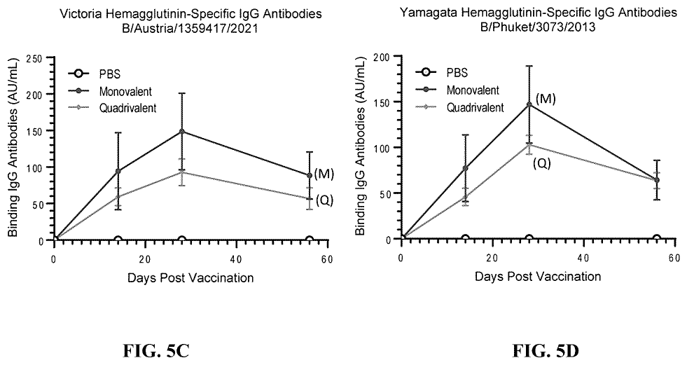

Female BALB/c mice were vaccinated with either 0.5 μg of the indicated monovalent saRNA (encoding NA and HA for the indicated strain in each of FIGS. 5A-5D) or a quadrivalent vaccine where each of the four saRNAs used for monovalent vaccines were mixed at a 1:1:1:1 ratio and then formulated. Titers of binding IgG antibodies against the indicated strain were measured in serum obtained at each of the indicated time points. FIG. 5A shows results for H1N1 A/Wisconsin/588/2019 influenza. FIG. 5B shows results for H3N2 A/Darwin/6/2021 influenza. FIG. 5C shows results for Victoria B/Austria/1359417/2021 influenza. FIG. 5D shows results for Yamagata B/Phuket/3073/2013 influenza.

The data indicates that for most strains, a drop in strain-specific HA antibody titers was observed for quadrivalent vaccines as compared to the corresponding monovalent vaccine. However, the drop was relatively small, and levels of antibodies to each antigen are comparable.

### Example 5

Immunogenicity and Reactogenicity in Formulations with Varying Nitrogen:Phosphate (N:P) Ratios

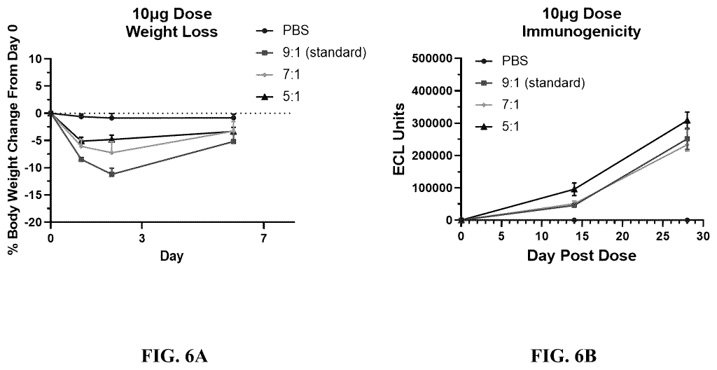

To evaluate the potential effect of the nitrogen to phosphate ratio in vaccine formulations on reactogenicity and tolerability, three N:P ratios were tested: 9:1, 7:1, and 5:1. Mice were vaccinated with a modified conventional mRNA encoding H1N1 A/California/07/2009 hemagglutinin formulated with an ionizable cationic lipid ATX-126 to achieve the indicated N:P ratios. The mice were tested for HA-specific antibodies (FIG. 6B), and weight loss as a measure of reactogenicity (FIG. 6A). Lower N:P ratios resulted in better-tolerated formulations while maintaining immunogenicity. The 7:1 ratio maintained a similar immunogenic profile as the 9:1 formulation. Therefore, further screening of lipids was performed at a 7:1 ratio.

### Example 6

Assessment Vaccine Formulations with Different Lipids

Tolerability and immunogenicity were evaluated for vaccine formulations that differed by amount of saRNA administered and and ionizable cationic lipid compound selected for the formulations. Tolerability was defined in mouse experiments as clinical observations (ruffled fur, activity, hunched posture) and weight loss. Formulations were first tested with modified mRNA, since modified mRNA produces better tolerability than unmodified RNA and thus assessment of the contributions of the lipid to tolerability could be more easily assessed. Top formulations were then tested with saRNA in which we observed similar results with modified conventional mRNA, except that differences in the context of a saRNA were smaller than those with modified conventional mRNA.

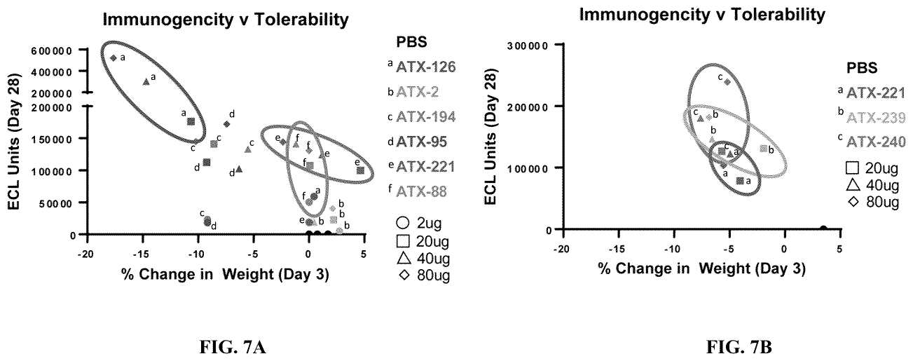

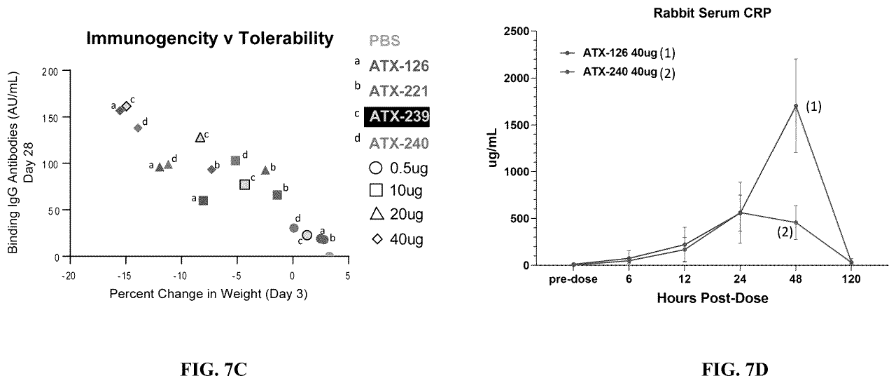

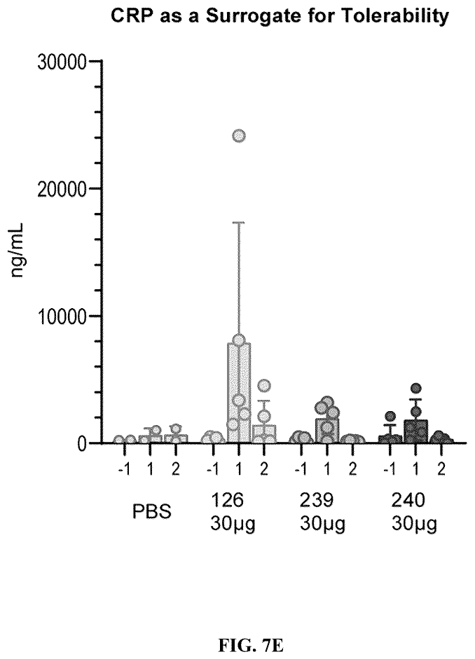

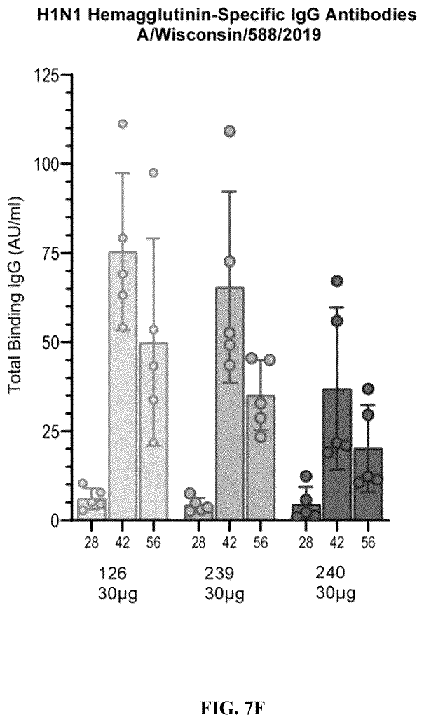

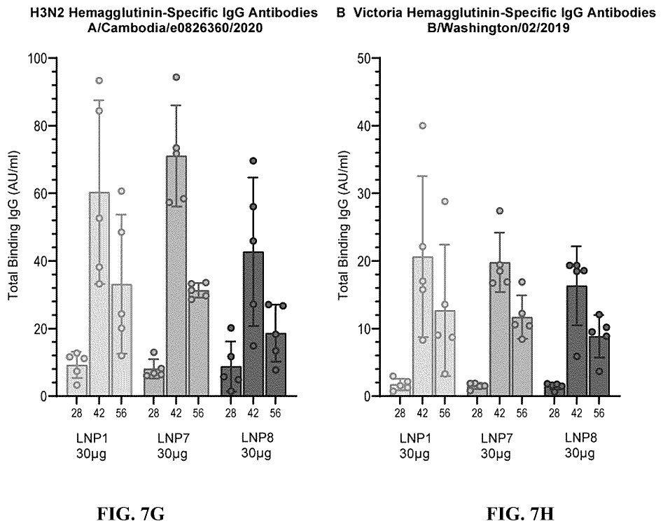

Female BALB/c mice were immunized with a modified conventional mRNA encoding the hemagglutinin from A/California/07/2009 formulated with different lipids at varying doses. Serum collected at day 28 was used to determine HA-specific antibodies while peak weight loss at day 3 was used as the measure of tolerability, and PBS was used as a control. Results for the indicated lipids and doses are shown in FIGS. 7A-7B. The results in FIGS. 7A-7B show HA-specific antibody levels in serum (reported in ECL unites) vs. percentage changes in body weight of the mice. Lipid compounds tested included ATX-126, ATX-2, ATX-194, ATX-95, ATX-221, ATX-88, ATX-239, and ATX-240. Vaccinations were given in 2 μg, 20 μg, 40 μg, or 80 μg doses. ATX-194 and ATX-95 showed comparable immunogenicity and tolerability (FIG. 7A; not circled, center), while ATX-2 showed the lowest immunogenicity with minimal variability in weight (FIG. 7A; not circled, lower right). ATX-126 remained the most immunogenic ionizable lipid (FIG. 7A), while formulations with ATX-240 and ATX-239 had acceptable immunogenicity and were better tolerated at lower levels (FIG. 7B).

A similar experiment was performed using saRNA instead of conventional mRNA with the top formulations. FIG. 7C shows levels of binding IgG antibodies in serum (AU/mL) vs. percentage changes in body weight of immunized female BALB/c mice. Lipids tested in the saRNA experiments included ATX-126, ATX-221, ATX-239, and ATX-240. Vaccinations were given in 0.5 μg, 10 μg, 20 μg, and 40 μg doses. Serum collected at day 28 was used to determine HA specific antibodies while weight loss (peaking at day 3) was used as the measure of tolerability. PBS was used as a control. All vaccines showed similar immunogenicity and tolerability at the lowest dose of 0.5 mg (FIG. 7C; circles, lower right of graph). At a dose of 40 mg, ATX-126, ATX-239, and ATX-240 showed the highest immunogenicity, with ATX-221 showing the best tolerability (FIG. 7C; diamonds). All vaccines showed similar immunogenicity at a dose of 20 mg, with ATX-221 showing the best tolerability (FIG. 7C; triangles). At a dose of 10 mg, similar immunogenicity was observed, with ATX-221 showing the best tolerability (FIG. 7C; squares).

Female New Zealand white rabbits were vaccinated with 40 μg saRNA formulated with either ATX-126 or ATX-240 to assess tolerability in the rabbits using the surrogate of c-reactive protein (CRP) as a marker for systemic reactogenicity. Measurements were taken from pre-dose to 120 hours post-dose. As shown in FIG. 7D, elevated CRP levels were observed at 48 hours in ATX-126 formulations with only a slight increase in ATX-240 formulations. Differences in CRP levels for different formulations were confirmed in a test replicated with non-human primates (NHP). FIG. 7E shows levels of c-reactive protein (CRP) in serum (ng/mL) of non-human primates (NHP) vaccinated with quadrivalent (encoding both HA and NA from 2021/22 recommended strains) modified conventional mRNA formulated with ATX-126, ATX-239, and ATX-240, and each at a dose of 30 μg. Elevated CRP levels were only observed for formulations with ATX-126. Formulations with ATX-239 and -240 with the same drug substance showed lower CRP 24 hours post dose.

Hemagglutinin-specific antibodies at Day 28 from the same NHP experiment indicated a similar immunogenic profile among the three formulations tested. FIGS. 7F-7H displays results for three of the four strains tested. Results report titers for strain-specific HA IgG antibodies for H1N1 A/Wisconsin/588/2019 (FIG. 7F), H3N2 A/Cambodia/e0826360/2020 (FIG. 7G), and B Victoria B/Washington/02/2019 (FIG. 7H). Results for a Yamagata strain were similar. The results indicate that the same trend observed in NHPs was also observed in mice, in which conventional mRNA formulated with ATX-240 was slightly less immunogenic than mRNA formulated with ATX-126.

### Example 7

### Multicistronic Design

To test the feasibility of higher-order multi-cistronic expression cassettes, saRNAs were prepared in which two saRNAs each encoded the HA and NA from two different strains (two tetra-cistronic constructs), for comparison to the bi-cistronic quadrivalent formulation and to conventional mRNA.

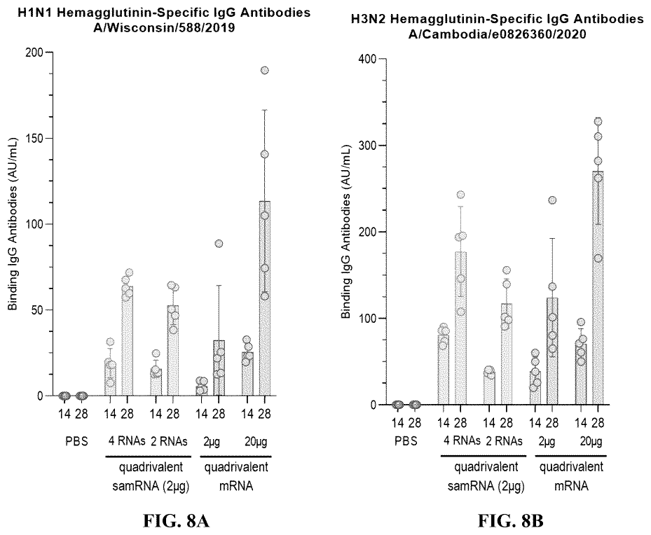

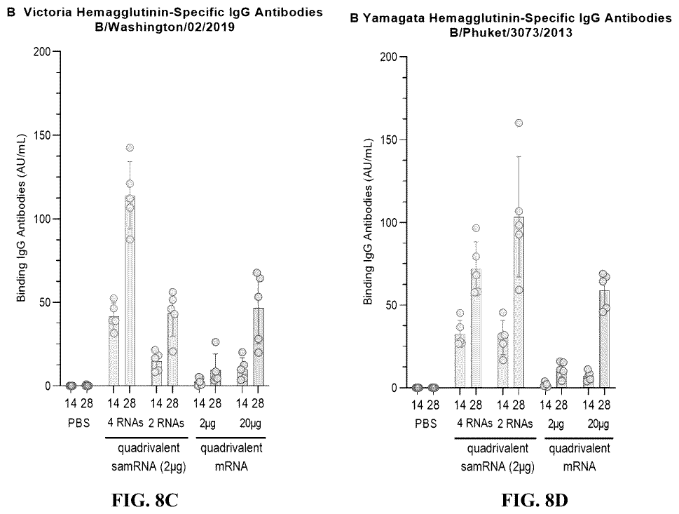

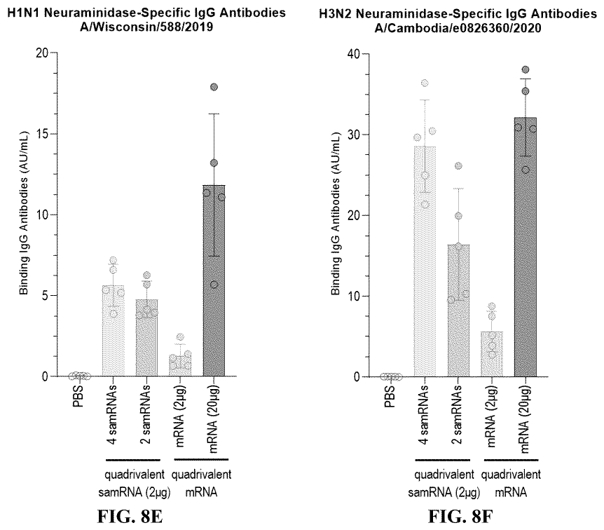

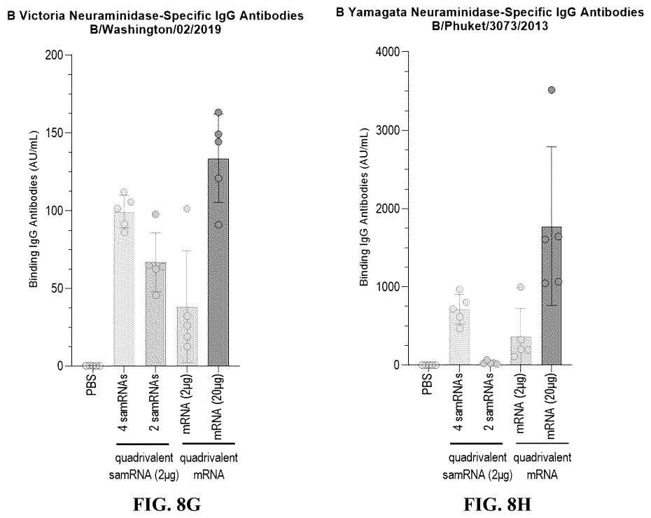

To prepare quadrivalent influenza vaccines, HA and NA from FDA-recommended strains for cell-based vaccines for the 2021/2022 Northern Hemisphere influenza season were encoded in a self-amplifying RNA (prepared in 2 μg doses) or conventional N1-pseudouridine-modified mRNA (prepared in 2 μg and 20 μg doses). Antigens in the conventional modified mRNA were encoded on separate RNAs and combined in equal amounts (by mass) during the lipid encapsulation process. NA and HA from each strain was encoded on the same saRNA (4 RNAs group, as in RNA schematic in FIG. 2) or 2 NAs and 2 HAs were encoded on the same saRNA (2 RNAs group, NA2-NA1-HA3-HA1 and NA (Victoria)-NA(Yamagata)-HA(Victoria)-HA-Yamagata)). Each antigen encoded by the saRNA is preceded by a subgenomic promoter. FIGS. 8A-8D show the HA-specific IgG antibody response for each influenza subtype (FIG. 8A: H1N1 A/Wisconsin/588/2019; FIG. 8B: H3N2 A/Cambodia/e0826360/2020; FIG. 8C: B Victoria B/Washington/02/2019; FIG. 8D: B Yamagata B/Phuket/3073/2013), while FIGS. 8E-8H show the NA-specific IgG antibody response for each influenza subtype (FIG. 8E: H1N1 A/Wisconsin/588/2019; FIG. 8F: H3N2 A/Cambodia/e0826360/2020; FIG. 8G: B Victoria B/Washington/02/2019; FIG. 8H: B Yamagata B/Phuket/3073/2013).

It was found that up to four influenza antigens could be encoded on an mRNA molecule. It was also found that when four antigens are encoded on one saRNA (“2samRNA” groups) that the response is lower than when only two antigens, one HA and one NA from the same strain, are encoded on one RNA (“4 samRNA” groups).

### Example 8

### Internal Ribosomal Entry Sites (IRES)

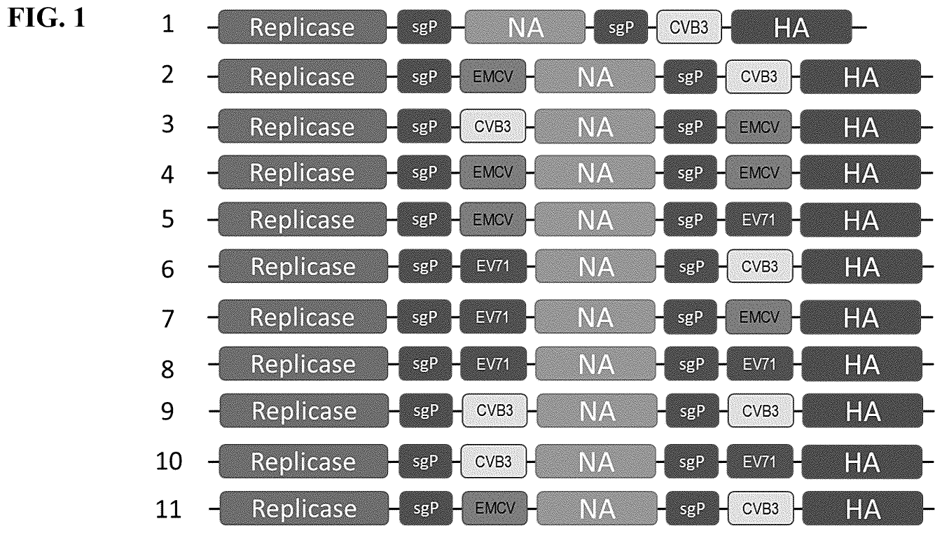

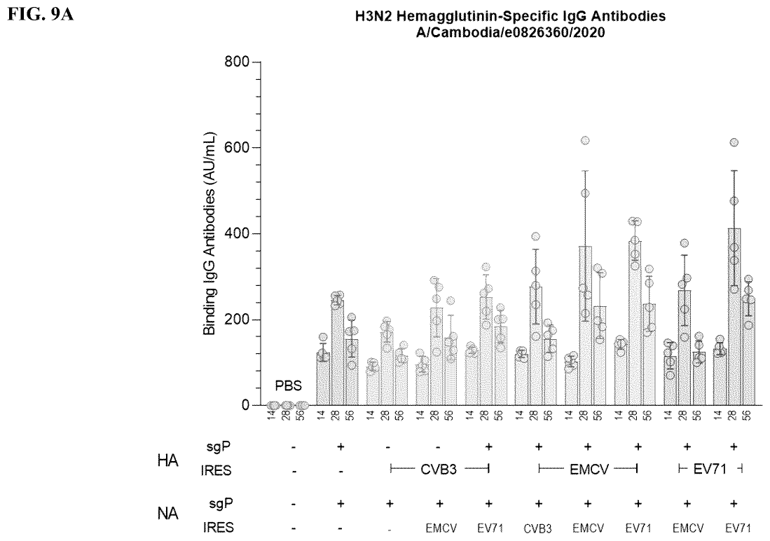

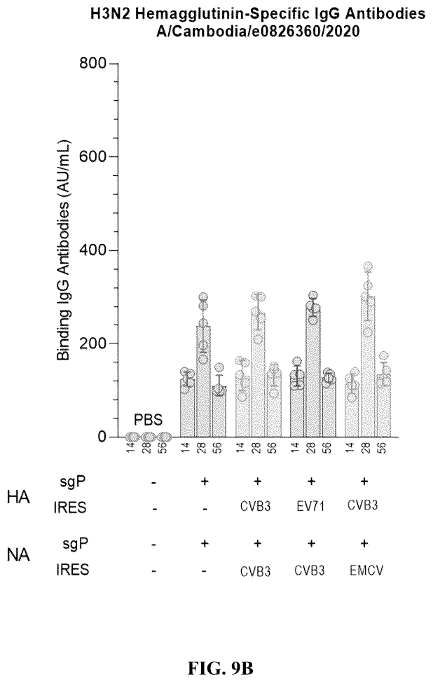

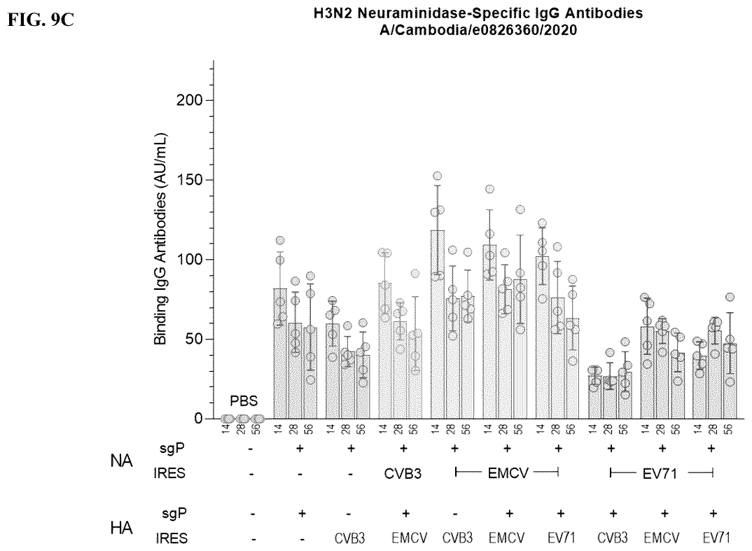

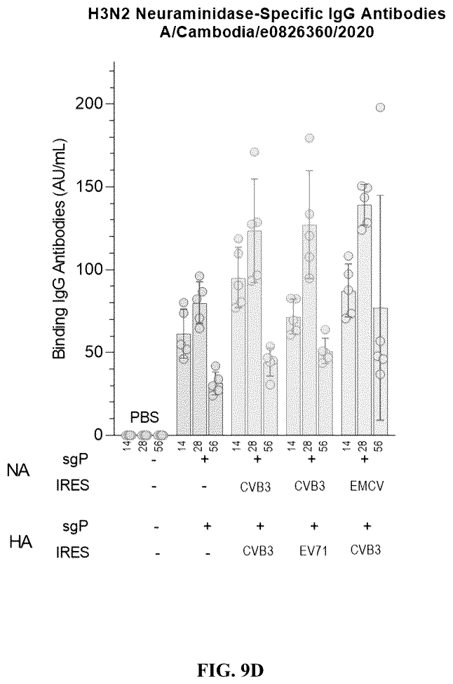

Internal ribosomal entry sites (IRES) from several organisms were tested in a bicistronic saRNA. IRES sequences were inserted 3′ to the subgenomic promoter (sgP) in various combinations. Schematics of constructs tested are illustrated in FIG. 1, and sequences for various constructs are represented by SEQ ID NOs: 151-161. Female BALB/c mice (8-10 weeks in age) were vaccinated with a single saRNA encoding both NA and HA of H3N2 A/Cambodia/e0826360/2020 where NA was in the “first” position and HA was in the “second” position. Subgenomic promoters were positioned 5′ to each antigen of interest except where indicated in each of FIGS. 9A-9D. IRES sequences were positioned between the sgP and the antigen of interest. FIGS. 9A-9B show hemagglutinin-specific IgG antibody responses from serum taken at the indicated days post vaccination (x-axis), while FIGS. 9C-9D show neuraminidase-specific IgG antibody responses from serum taken at the indicated days post vaccination (x-axis).

While constructs where EMCV was positioned between the sgP and the antigen of interest generally showed slightly better responses, these responses were found to not be significant with the number of mice used. Further, the results indicate that the antigens preceded by the EMC IRES were typically slightly more immunogenic than those without the IRES sequence, however, these differences were not significant.

### SEQUENCES

For SEQ ID NOs. 1-4: Italicized text denotes nsP1-4 replicase sequences; bold text denotes NA sequences; bold and underlined text denotes HA sequences.

SEQ ID NO: 1 (H1N1 bicistronic construct; self-replicating RNA for expression
of NA/HA from H1N1 influenza strain)
ATGGGCGGCGCATGAGAGAAGCCCAGACCAATTACCTACCCAAAATGGAGAAAGTTCACGTT
GACATCGAGGAAGACAGCCCATTCCTCAGAGCTTTGCAGCGGAGCTTCCCGCAGTTTGAGGTAGA
AGCCAAGCAGGTCACTGATAATGACCATGCTAATGCCAGAGCGTTTTCGCATCTGGCTTCAAAACT
GATCGAAACGGAGGTGGACCCATCCGACACGATCCTTGACATTGGAAGTGCGCCCGCCCGCAGA
ATGTATTCTAAGCACAAGTATCATTGTATCTGTCCGATGAGATGTGCGGAAGATCCGGACAGATTG
TATAAGTATGCAACTAAGCTGAAGAAAAACTGTAAGGAAATAACTGATAAGGAATTGGACAAGAAAA
TGAAGGAGCTGGCCGCCGTCATGAGCGACCCTGACCTGGAAACTGAGACTATGTGCCTCCACGA
CGACGAGTCGTGTCGCTACGAAGGGCAAGTCGCTGTTTACCAGGATGTATACGCCGTCGACGGC
CCCACCAGCCTGTACCACCAGGCCAACAAGGGCGTGAGGGTGGCCTACTGGATCGGCTTCGACA
CCACACCCTTCATGTTCAAGAACCTGGCCGGCGCCTACCCCAGCTACAGCACCAACTGGGCCGAC
GAGACAGTGCTGACCGCCAGGAACATCGGCCTGTGCAGCAGCGACGTGATGGAGAGGAGCCGG
AGGGGCATGAGCATCCTGAGGAAGAAGTACCTGAAGCCCAGCAACAACGTGCTGTTCAGCGTGG
GCAGCACCATCTACCACGAGAAGAGGGACCTGCTGAGGAGCTGGCACCTGCCCAGCGTGTTCCA
CCTGAGGGGCAAGCAGAACTACACCTGCAGGTGCGAGACAATCGTGAGCTGCGACGGCTACGTG
GTGAAGAGGATCGCCATCAGCCCCGGCCTGTACGGCAAGCCCAGCGGCTACGCCGCCACCATGC
ACAGGGAGGGCTTCCTGTGCTGCAAGGTGACCGACACCCTGAACGGCGAGAGGGTGAGCTTCCC
CGTGTGCACCTACGTGCCCGCCACCCTGTGCGACCAGATGACCGGCATCCTGGCCACCGACGTG
AGCGCCGACGACGCCCAGAAGCTGCTGGTGGGCCTGAACCAGAGGATCGTGGTGAACGGCAGG
ACCCAGAGGAACACCAACACCATGAAGAACTACCTGCTGCCCGTGGTGGCCCAGGCCTTCGCCA
GGTGGGCCAAGGAGTACAAGGAGGACCAGGAGGACGAGAGGCCCCTGGGCCTGAGGGACCGAC
AGCTGGTGATGGGCTGCTGCTGGGCCTTCAGGCGGCACAAGATCACCAGCATCTACAAGAGGCC
CGACACCCAGACCATCATCAAGGTGAACAGCGACTTCCACAGCTTCGTGCTGCCCAGGATCGGCA
GCAACACCCTGGAGATCGGCCTGAGGACCCGGATCAGGAAGATGCTGGAGGAGCACAAGGAGCC
CAGCCCTCTGATCACCGCCGAGGACGTGCAGGAGGCCAAGTGCGCCGCCGACGAGGCCAAGGA
GGTGAGGGAGGCCGAGGAGCTGAGGGCCGCCCTGCCTCCCCTGGCCGCCGACGTGGAGGAGC
CCACCCTGGAGGCCGACGTGGACCTGATGCTGCAGGAGGCCGGCGCCGGCAGCGTGGAGACAC
CCAGGGGCCTGATCAAGGTGACCAGCTACGACGGCGAGGACAAGATCGGCAGCTACGCCGTGCT
CAGCCCTCAGGCCGTGCTGAAGTCCGAGAAGCTGAGCTGCATCCACCCTCTGGCCGAGCAGGTG
ATCGTGATCACCCACAGCGGCAGGAAGGGCAGGTACGCCGTGGAGCCCTACCACGGCAAGGTG
GTGGTCCCCGAGGGCCACGCCATCCCCGTGCAGGACTTCCAGGCCCTGAGCGAGAGCGCCACC
ATCGTGTATAACGAGAGGGAGTTCGTGAACAGGTACCTGCACCACATCGCCACCCACGGCGGCG
CCCTGAACACCGACGAGGAGTACTACAAGACCGTGAAGCCCAGCGAGCACGACGGCGAGTACCT
GTACGACATCGACAGGAAGCAGTGCGTGAAGAAGGAGCTGGTGACCGGCCTGGGCCTGACCGG
CGAGCTGGTGGACCCTCCCTTCCACGAGTTCGCCTACGAGAGCCTGAGGACCAGGCCCGCCGCT
CCCTACCAGGTGCCCACCATCGGCGTGTACGGCGTGCCCGGCAGCGGCAAGAGCGGCATCATCA
AGAGCGCCGTGACCAAGAAGGACCTGGTGGTGAGCGCCAAGAAGGAGAACTGCGCCGAGATCAT
CAGGGACGTGAAGAAGATGAAGGGCCTGGACGTGAACGCCAGGACCGTGGACAGCGTGCTCCTG
AACGGCTGCAAGCACCCCGTGGAGACACTGTATATCGACGAGGCCTTCGCCTGCCACGCCGGCA
CCCTGAGGGCCCTGATCGCCATCATCAGGCCCAAGAAGGCCGTGCTGTGCGGCGACCCCAAGCA
GTGCGGCTTCTTCAACATGATGTGCCTGAAGGTGCACTTCAACCACGAGATCTGCACCCAGGTGTT
CCACAAGAGCATCAGCAGGCGGTGCACCAAGAGCGTGACCAGCGTGGTGAGCACCCTGTTCTAC
GACAAGAAGATGAGGACCACCAACCCCAAGGAGACAAAGATCGTGATCGACACCACCGGCAGCA
CCAAGCCCAAGCAGGACGACCTGATCCTGACCTGCTTCAGGGGCTGGGTGAAGCAGCTGCAGAT
CGACTACAAGGGCAACGAGATCATGACCGCCGCCGCTAGCCAGGGCCTGACCAGGAAGGGCGT
GTACGCCGTGAGGTACAAGGTGAACGAGAATCCCCTGTACGCCCCTACCAGCGAGCACGTGAAC
GTCCTGCTGACCAGGACCGAGGACAGGATCGTGTGGAAGACCCTGGCCGGCGACCCCTGGATCA
AGACCCTGACCGCCAAGTACCCCGGCAACTTCACCGCCACCATCGAGGAGTGGCAGGCCGAGCA
CGACGCCATCATGAGGCACATCCTGGAGAGGCCCGACCCCACCGACGTGTTCCAGAACAAGGCC
AACGTGTGCTGGGCCAAGGCCCTGGTGCCCGTGCTGAAGACCGCCGGCATCGACATGACCACCG
AGCAGTGGAACACCGTGGACTACTTCGAGACAGACAAGGCCCACAGCGCCGAGATCGTGCTGAA
CCAGCTGTGCGTGAGGTTCTTCGGCCTGGACCTGGACAGCGGCCTGTTCAGCGCCCCTACCGTG
CCCCTGAGCATCAGGAACAACCACTGGGACAACAGCCCCAGCCCCAACATGTACGGCCTGAACAA
GGAGGTGGTGAGGCAGCTGAGCAGGCGGTACCCTCAGCTGCCCAGGGCCGTGGCCACCGGCAG
GGTGTACGACATGAACACCGGCACCCTGAGGAACTACGACCCCAGGATCAACCTGGTGCCCGTG
AACAGGCGGCTGCCACACGCCCTGGTGCTGCACCACAACGAGCACCCTCAGAGCGACTTCAGCA
GCTTCGTGAGCAAGCTGAAGGGCAGGACCGTGCTGGTGGTGGGCGAGAAGCTGAGCGTGCCCG
GCAAGATGGTGGACTGGCTGAGCGACAGGCCCGAGGCCACCTTCCGGGCCAGGCTGGACCTGG
GCATCCCCGGCGACGTGCCCAAGTACGACATCATCTTCGTGAACGTGAGGACCCCTTACAAGTAC
CACCACTACCAGCAGTGCGAGGACCACGCCATCAAGCTGAGCATGCTGACCAAGAAGGCCTGCC
TGCACCTGAACCCCGGCGGCACCTGCGTGAGCATCGGCTACGGCTACGCCGACAGGGCCAGCG
AGAGCATCATCGGCGCCATCGCCAGGCTGTTCAAGTTCAGCAGGGTGTGCAAGCCCAAGAGCAG
CCTGGAGGAGACAGAGGTGCTGTTCGTGTTCATCGGCTACGACCGGAAGGCCAGGACCCACAAC
CCCTACAAGCTGAGCAGCACCCTGACCAACATCTACACCGGCAGCAGGCTGCACGAGGCCGGCT
GCGCCCCTAGCTACCACGTGGTGAGGGGCGACATCGCCACCGCCACCGAGGGCGTGATCATCAA
CGCCGCCAACAGCAAGGGCCAGCCCGGCGGCGGGGTGTGCGGCGCCCTGTATAAGAAGTTCCC
CGAGAGCTTCGACCTGCAGCCCATCGAGGTGGGCAAGGCCAGGCTGGTGAAGGGCGCCGCCAA
GCACATCATCCACGCCGTGGGCCCCAACTTCAACAAGGTGAGCGAGGTGGAGGGCGACAAGCAG
CTGGCCGAGGCCTACGAGAGCATCGCCAAGATCGTGAACGACAACAACTACAAGAGCGTGGCCA
TCCCTCTGCTGAGCACCGGCATCTTCAGCGGCAACAAGGACAGGCTGACCCAGAGCCTGAACCA
CCTGCTGACCGCCCTGGACACCACCGACGCCGACGTGGCCATCTACTGCAGGGACAAGAAGTGG
GAGATGACCCTGAAGGAGGCCGTGGCCAGGCGGGAGGCCGTGGAGGAGATCTGCATCAGCGAC
GACAGCAGCGTGACGGAGCCCGACGCCGAGCTGGTGAGGGTGCACCCCAAGAGCAGCCTGGCC
GGCAGGAAGGGCTACAGCACCAGCGACGGCAAGACCTTCAGCTACCTGGAGGGCACCAAGTTCC
ACCAGGCCGCCAAGGACATCGCCGAGATCAACGCCATGTGGCCCGTGGCCACCGAGGCCAACGA
GCAGGTGTGCATGTATATCCTGGGCGAGAGCATGAGCAGCATCAGGAGCAAGTGCCCCGTGGAG
GAGAGCGAGGCCAGCACCCCTCCCAGCACCCTGCCCTGCCTGTGCATCCACGCCATGACCCCTG
AGAGGGTGCAGCGGCTGAAGGCCAGCAGGCCCGAGCAGATCACCGTGTGCAGCAGCTTCCCTCT
GCCCAAGTACCGGATCACCGGCGTGCAGAAGATCCAGTGCAGCCAGCCCATCCTGTTCAGCCCC
AAGGTGCCCGCCTACATCCACCCCAGGAAGTACCTGGTGGAGACACCCCCCGTGGACGAGACAC
CCGAGCCCAGCGCCGAGAACCAGAGCACCGAGGGCACCCCTGAGCAGCCTCCCCTGATCACCG
AGGACGAGACAAGGACCAGGACGCCCGAGCCCATCATCATTGAGGAGGAAGAGGAGGACAGCAT
CAGCCTGCTGAGCGACGGCCCCACCCACCAGGTGCTGCAGGTGGAGGCCGACATCCACGGCCC
TCCCAGCGTGAGCAGCTCCAGCTGGAGCATCCCTCACGCCAGCGACTTCGACGTGGACAGCCTG
AGCATCCTGGACACCCTGGAGGGCGCCAGCGTGACCAGCGGCGCCACCAGCGCCGAGACAAAC
AGCTACTTCGCCAAGAGCATGGAGTTCCTGGCCAGGCCCGTGCCCGCCCCTAGGACCGTGTTCA
GGAACCCTCCCCACCCCGCCCCTAGGACCAGGACCCCTAGCCTGGCCCCTAGCAGGGCCTGCAG
CAGGACCAGCCTGGTGAGCACCCCTCCCGGCGTGAACCGGGTGATCACCAGGGAGGAGCTGGA
GGCCCTGACCCCTAGCAGGACCCCTAGCAGGAGCGTGAGCAGGACCAGCCTGGTGAGCAACCCT
CCCGGCGTGAACCGGGTGATCACCAGGGAGGAGTTCGAGGCCTTCGTGGCCCAGCAGCAAAGG
CGGTTCGACGCCGGCGCCTACATCTTCAGCAGCGACACCGGCCAGGGCCACCTGCAGCAGAAGT
CCGTGAGGCAGACCGTGCTGAGCGAGGTGGTCCTGGAGAGGACGGAGCTGGAGATCAGCTACG
CCCCTAGGCTGGACCAGGAGAAGGAGGAGCTGCTGAGGAAGAAGCTGCAGCTGAACCCCACCCC
TGCCAACAGGAGCAGGTACCAGAGCAGGAAGGTGGAGAACATGAAGGCCATCACCGCCAGGCGG
ATCCTGCAGGGCCTGGGCCACTACCTGAAGGCCGAGGGCAAGGTGGAGTGCTACAGGACCCTGC
ACCCCGTGCCCCTGTACTCCAGCTCCGTGAACAGGGCCTTCAGCAGCCCCAAGGTGGCCGTGGA
GGCCTGCAACGCCATGCTGAAGGAGAACTTCCCCACCGTGGCCAGCTACTGCATCATCCCCGAGT
ACGACGCCTACCTGGACATGGTGGACGGCGCCAGCTGCTGCCTGGACACCGCCAGCTTCTGCCC
CGCCAAGCTGAGGAGCTTCCCCAAGAAGCACAGCTACCTGGAGCCCACCATCAGGAGCGCCGTG
CCCAGCGCCATCCAGAACACCCTGCAGAACGTGCTGGCCGCCGCTACCAAGAGGAACTGCAACG
TGACCCAGATGAGGGAGCTGCCCGTGCTGGACAGCGCCGCCTTCAACGTGGAGTGCTTCAAGAA
GTACGCCTGCAACAACGAGTACTGGGAGACATTCAAGGAGAACCCCATCAGGCTGACCGAGGAG
AACGTGGTGAACTACATCACCAAGCTGAAGGGCCCCAAGGCCGCCGCTCTGTTCGCCAAGACCCA
CAACCTGAACATGCTCCAGGACATCCCTATGGACAGGTTCGTGATGGACCTGAAGAGGGACGTGA
AGGTGACCCCTGGCACCAAGCACACCGAGGAGAGGCCCAAGGTGCAGGTGATCCAGGCCGCCG
ACCCTCTGGCCACCGCCTACCTGTGCGGCATCCACAGGGAGCTGGTGAGGCGGCTGAACGCCGT
CCTGCTGCCCAACATCCACACCCTGTTCGACATGAGCGCCGAGGACTTCGACGCCATCATCGCCG
AGCACTTCCAGCCCGGCGACTGCGTGCTGGAGACAGACATCGCCAGCTTCGACAAGAGCGAGGA
CGACGCTATGGCCCTGACCGCCCTGATGATCCTGGAGGACCTGGGCGTGGACGCCGAGCTGCTG
ACCCTGATCGAGGCCGCCTTCGGCGAGATCAGCAGCATCCACCTGCCCACCAAGACCAAGTTCAA
GTTCGGCGCCATGATGAAGTCCGGCATGTTCCTGACCCTGTTCGTGAACACCGTGATCAACATCG
TGATCGCCAGCAGGGTGCTGCGGGAGAGGCTGACCGGCAGCCCCTGCGCCGCCTTCATCGGCG
ACGACAACATCGTGAAGGGCGTGAAGTCCGACAAGCTGATGGCCGACAGGTGCGCCACCTGGCT
GAACATGGAGGTGAAGATCATCGACGCCGTGGTGGGCGAGAAGGCCCCTTACTTCTGCGGCGGC
TTCATCCTGTGCGACAGCGTGACCGGCACCGCCTGCAGGGTGGCCGACCCTCTGAAGAGGCTGT
TCAAGCTGGGCAAGCCCCTGGCCGCCGACGACGAGCACGACGACGATAGGCGGAGGGCCCTGC
ACGAGGAGAGCACCAGGTGGAACCGGGTGGGCATCCTGAGCGAGCTGTGCAAGGCCGTGGAGA
GCAGGTACGAGACAGTGGGCACCAGCATCATCGTGATGGCCATGACCACCCTGGCCAGCAGCGT
CAAGTCCTTCAGCTACCTGAGGGGGGCCCCTATAACTCTCTACGGCTAACCTGAATGGACTACG
ACATAGTCTAGTCCGCCAAGGCCGCCACCATGCTGCCTAGCACCATCCAGACCCTGACAC
TGTTCCTGACCAGCGGCGGCGTGCTGCTGAGCCTGTACGTGAGCGCCAGCCTGAGCT
ACCTGCTGTACTCCGACATCCTGCTGAAGTTCAGCCAGACCGAGATCACCGCCCCTAC
CATGCCCCTGGACTGCGCCAACGCCAGCAACGTGCAGGCCGTGAACAGGAGCGCCAC
CAAGGGCGCCACCCTGCTGCTCCCCGAGCCCGAGTGGACCTACCCCAGGCTGAGCTG
CCCCGGCAGCACCTTCCAGAAGGCCCTGCTGATCAGCCCTCACAGGTTCGGCGAGACA
AAGGGCAACAGCGCCCCTCTGATCATCAGGGAGCCCTTCGTGGCCTGCGGCCCCAAC
GAGTGCAAGCACTTCGCCCTGACCCACTACGCCGCCCAGCCCGGCGGCTACTACAACG
GCACCAGGGGCGACAGGAACAAGCTGAGGCACCTGATCAGCGTGAAGCTGGGCAAGA
TCCCCACCGTGGAGAACAGCATCTTCCACATGGCCGCCTGGAGCGGCAGCGCCTGCCA
CGACGGCAAGGAGTGGACCTACATCGGCGTGGACGGCCCCGACAACAACGCCCTGCT
GAAGGTGAAGTACGGCGAGGCCTACACCGACACCTACCACAGCTACGCCAACAACATC
CTGAGGACCCAGGAGAGCGCCTGCAACTGCATCGGCGGCAACTGCTACCTGATGATCA
CCGACGGCAGCGCCAGCGGCGTGAGCGAGTGCAGGTTCCTGAAGATCAGGGAGGGCA
GGATCATCAAGGAGATCTTCCCCACCGGCAGGGTGAAGCACACCGAGGAGTGCACCT
GCGGCTTCGCCAGCAACAAGACCATCGAGTGCGCCTGCAGGGACAACAGGTACACCG
CCAAGAGGCCCTTCGTGAAGCTGAACGTGGAGACAGACACCGCCGAGATCAGGCTGA
TGTGCACCGACACCTACCTGGACACCCCTAGGCCCAACGACGGCAGCATCACCGGCCC
CTGCGAGAGCGACGGCGACGAGGGCAGCGGCGGCATCAAGGGCGGCTTCGTGCACCA
GAGGATGAAGTCCAAGATCGGCAGGTGGTACAGCAGGACCATGAGCAAGACCGAGAG
GATGGGCATGGGCCTGTACGTGAAGTACGGCGGCGACCCCTGGGCCGACAGCGACGC
CCTGGTGTTCAGCGGCGTGATGATCAGCATGAAGGAGCCCGGCTGGTACAGCTTCGG
CTTCGAGATCAAGGACAAGAAGTGCGACGTGCCCTGCATCGGCATCGAGATGGTGCAC
GACGGCGGCAAGGAGACATGGCACAGCGCCGCCACCGCCATCTACTGCCTGATGGGC
AGCGGCCAGCTGCTGTGGGACACCATCACCGGCGTGGACATGGCCCTGTGATAGTAA
ACTCGAAGCAGTGTTAAATCATTCAGCTACCTGAGAGGGGCCCCTATAACTCTCTACGGCTA
ACCTGAATGGACTACGACATAGTCTAGTCCGCCAAGGCCGCCACCATGAAGGCCATCATC
GTGCTGCTGATGGTGGTGACCTCTAACGCCGACAGGATCTGCACCGGCATCACCAGCA
GCAACAGCCCTCACGTGGTGAAGACCGCCACCCAGGGCGAGGTGAACGTGACCGGCG
TGATCCCTCTGACCACCACACCCACCAAGAGCCACTTCGCCAACCTGAAGGGCACCGA
GACAAGGGGCAAGCTGTGCCCCAAGTGCCTGAACTGCACCGACCTGGACGTGGCCCT
GGGCAGGCCCAAGTGCACCGGCAAGATCCCCAGCGCCAGGGTGAGCATCCTGCACGA
GGTGAGGCCCGTGACCAGCGGCTGCTTCCCCATCATGCACGACAGGACCAAGATCAG
GCAGCTGCCCAACCTGCTGAGGGGCTACGAGCACGTGAGGCTGAGCACCCACAACGT
GATCAACACCGAGGACGCCCCTGGCGGCCCCTACGAGATCGGCACCAGCGGCAGCTG
CCTGAACATCACCAACGGCAAGGGCTTCTTCGCCACAATGGCCTGGGCCGTGCCCAAG
AACAAGACCGCCACCAATCCCCTGACCATCGAGGTGCCCTACATCTGCACCGAGGAGG
AGGACCAGATCACCGTGTGGGGCTTCCACAGCGACGACGAGACACAGATGGCCAGGC
TGTACGGCGACAGCAAGCCCCAGAAGTTCACCAGCAGCGCCAACGGCGTGACCACCC
ACTACGTGAGCCAGATCGGCGGCTTCCCCAACCAGACCGAGGACGGCGGCCTGCCCC
AGAGCGGCAGGATCGTGGTGGACTACATGGTGCAGAAGTCCGGCAAGACCGGCACCA
TCACCTACCAGAGGGGCATCCTGCTGCCCCAGAAGGTGTGGTGCGCCAGCGGCAAGA
GCAAGGTGATCAAGGGCAGCCTGCCCCTGATCGGCGAGGCCGACTGCCTGCACGAGA
AGTACGGCGGCCTGAACAAGAGCAAGCCCTACTACACCGGCGAGCACGCCAAGGCCA
TCGGCAACTGCCCCATCTGGGTGAAGACCCCTCTGAAGCTGGCCAACGGCACCAAGTA
CAGGCCTCCCGCCAAGCTGCTGAAGGAGAGGGGCTTCTTCGGCGCCATCGCCGGCTT
CCTGGAGGGCGGCTGGGAGGGCATGATCGCCGGCTGGCACGGCTACACCAGCCACGG
CGCCCACGGCGTGGCCGTGGCCGCCGACCTGAAGTCCACCCAGGAGGCCATCAACAA
GATCACCAAGAACCTGAACAGCCTGAGCGAGCTGGAGGTGAAGAACCTGCAGAGGCT
GAGCGGCGCTATGGACGAGCTGCACAACGAGATCCTGGAGCTGGACGAGAAGGTGGA
CGACCTGAGGGCCGACACCATCAGCAGCCAGATCGAGCTGGCCGTGCTGCTGAGCAA
CGAGGGCATCATCAACAGCGAGGACGAGCACCTGCTGGCCCTGGAGAGGAAGCTGAA
GAAGATGCTGGGCCCCAGCGCCGTGGAGATCGGCAACGGCTGCTTCGAGACAAAGCA
CAAGTGCAACCAGACCTGCCTGGACAGGATCGCCGCCGGCACCTTCGACGCCGGCGA
GTTCAGCCTGCCCACCTTCGACAGCCTGAACATCACCGCCGCCAGCCTGAACGACGAC
GGCCTGGACAACCACACCATCCTGCTGTACTACAGCACCGCCGCCAGCAGCCTGGCCG
TGACCCTGATGATCGCCATCTTCGTGGTGTATATGGTGAGCAGGGACAACGTGAGCTG
CAGCATCTGCCTGTGATAGTAAACTCGAGTATGTTACGTGCAAAGGTGATTGTCACCCCCC
GAAAGACCATATTGTGACACACCCTCAGTATCACGCCCAAACATTTACAGCCGCGGTGTCA
AAAACCGCGTGGACGTGGTTAACATCCCTGCTGGGAGGATCAGCCGTAATTATTATAATTGG
CTTGGTGCTGGCTACTATTGTGGCCATGTACGTGCTGACCAACCAGAAACATAATTGAATAC
AGCAGCAATTGGCAAGCTGCTTACATAGAACTCGCGGCGATTGGCATGCCGCCTTAAAATTT
TTATTTTATTTTTTCTTTTCTTTTCCGAATCGGATTTTGTTTTTAATATTTC
SEQ ID NO: 2 (H3N2 bicistronic construct; self-replicating RNA for expression
of NA/HA from H3N2 influenza strain)
ATGGGCGGCGCATGAGAGAAGCCCAGACCAATTACCTACCCAAAATGGAGAAAGTTCACGTT
GACATCGAGGAAGACAGCCCATTCCTCAGAGCTTTGCAGCGGAGCTTCCCGCAGTTTGAGGTAGA
AGCCAAGCAGGTCACTGATAATGACCATGCTAATGCCAGAGCGTTTTCGCATCTGGCTTCAAAACT
GATCGAAACGGAGGTGGACCCATCCGACACGATCCTTGACATTGGAAGTGCGCCCGCCCGCAGA
ATGTATTCTAAGCACAAGTATCATTGTATCTGTCCGATGAGATGTGCGGAAGATCCGGACAGATTG
TATAAGTATGCAACTAAGCTGAAGAAAAACTGTAAGGAAATAACTGATAAGGAATTGGACAAGAAAA
TGAAGGAGCTGGCCGCCGTCATGAGCGACCCTGACCTGGAAACTGAGACTATGTGCCTCCACGA
CGACGAGTCGTGTCGCTACGAAGGGCAAGTCGCTGTTTACCAGGATGTATACGCCGTCGACGGC
CCCACCAGCCTGTACCACCAGGCCAACAAGGGCGTGAGGGTGGCCTACTGGATCGGCTTCGACA
CCACACCCTTCATGTTCAAGAACCTGGCCGGCGCCTACCCCAGCTACAGCACCAACTGGGCCGAC
GAGACAGTGCTGACCGCCAGGAACATCGGCCTGTGCAGCAGCGACGTGATGGAGAGGAGCCGG
AGGGGCATGAGCATCCTGAGGAAGAAGTACCTGAAGCCCAGCAACAACGTGCTGTTCAGCGTGG
GCAGCACCATCTACCACGAGAAGAGGGACCTGCTGAGGAGCTGGCACCTGCCCAGCGTGTTCCA
CCTGAGGGGCAAGCAGAACTACACCTGCAGGTGCGAGACAATCGTGAGCTGCGACGGCTACGTG
GTGAAGAGGATCGCCATCAGCCCCGGCCTGTACGGCAAGCCCAGCGGCTACGCCGCCACCATGC
ACAGGGAGGGCTTCCTGTGCTGCAAGGTGACCGACACCCTGAACGGCGAGAGGGTGAGCTTCCC
CGTGTGCACCTACGTGCCCGCCACCCTGTGCGACCAGATGACCGGCATCCTGGCCACCGACGTG
AGCGCCGACGACGCCCAGAAGCTGCTGGTGGGCCTGAACCAGAGGATCGTGGTGAACGGCAGG
ACCCAGAGGAACACCAACACCATGAAGAACTACCTGCTGCCCGTGGTGGCCCAGGCCTTCGCCA
GGTGGGCCAAGGAGTACAAGGAGGACCAGGAGGACGAGAGGCCCCTGGGCCTGAGGGACCGAC
AGCTGGTGATGGGCTGCTGCTGGGCCTTCAGGCGGCACAAGATCACCAGCATCTACAAGAGGCC
CGACACCCAGACCATCATCAAGGTGAACAGCGACTTCCACAGCTTCGTGCTGCCCAGGATCGGCA
GCAACACCCTGGAGATCGGCCTGAGGACCCGGATCAGGAAGATGCTGGAGGAGCACAAGGAGCC
CAGCCCTCTGATCACCGCCGAGGACGTGCAGGAGGCCAAGTGCGCCGCCGACGAGGCCAAGGA
GGTGAGGGAGGCCGAGGAGCTGAGGGCCGCCCTGCCTCCCCTGGCCGCCGACGTGGAGGAGC
CCACCCTGGAGGCCGACGTGGACCTGATGCTGCAGGAGGCCGGCGCCGGCAGCGTGGAGACAC
CCAGGGGCCTGATCAAGGTGACCAGCTACGACGGCGAGGACAAGATCGGCAGCTACGCCGTGCT
CAGCCCTCAGGCCGTGCTGAAGTCCGAGAAGCTGAGCTGCATCCACCCTCTGGCCGAGCAGGTG
ATCGTGATCACCCACAGCGGCAGGAAGGGCAGGTACGCCGTGGAGCCCTACCACGGCAAGGTG
GTGGTCCCCGAGGGCCACGCCATCCCCGTGCAGGACTTCCAGGCCCTGAGCGAGAGCGCCACC
ATCGTGTATAACGAGAGGGAGTTCGTGAACAGGTACCTGCACCACATCGCCACCCACGGCGGCG
CCCTGAACACCGACGAGGAGTACTACAAGACCGTGAAGCCCAGCGAGCACGACGGCGAGTACCT
GTACGACATCGACAGGAAGCAGTGCGTGAAGAAGGAGCTGGTGACCGGCCTGGGCCTGACCGG
CGAGCTGGTGGACCCTCCCTTCCACGAGTTCGCCTACGAGAGCCTGAGGACCAGGCCCGCCGCT
CCCTACCAGGTGCCCACCATCGGCGTGTACGGCGTGCCCGGCAGCGGCAAGAGCGGCATCATCA
AGAGCGCCGTGACCAAGAAGGACCTGGTGGTGAGCGCCAAGAAGGAGAACTGCGCCGAGATCAT
CAGGGACGTGAAGAAGATGAAGGGCCTGGACGTGAACGCCAGGACCGTGGACAGCGTGCTCCTG
AACGGCTGCAAGCACCCCGTGGAGACACTGTATATCGACGAGGCCTTCGCCTGCCACGCCGGCA
CCCTGAGGGCCCTGATCGCCATCATCAGGCCCAAGAAGGCCGTGCTGTGCGGCGACCCCAAGCA
GTGCGGCTTCTTCAACATGATGTGCCTGAAGGTGCACTTCAACCACGAGATCTGCACCCAGGTGTT
CCACAAGAGCATCAGCAGGCGGTGCACCAAGAGCGTGACCAGCGTGGTGAGCACCCTGTTCTAC
GACAAGAAGATGAGGACCACCAACCCCAAGGAGACAAAGATCGTGATCGACACCACCGGCAGCA
CCAAGCCCAAGCAGGACGACCTGATCCTGACCTGCTTCAGGGGCTGGGTGAAGCAGCTGCAGAT
CGACTACAAGGGCAACGAGATCATGACCGCCGCCGCTAGCCAGGGCCTGACCAGGAAGGGCGT
GTACGCCGTGAGGTACAAGGTGAACGAGAATCCCCTGTACGCCCCTACCAGCGAGCACGTGAAC
GTCCTGCTGACCAGGACCGAGGACAGGATCGTGTGGAAGACCCTGGCCGGCGACCCCTGGATCA
AGACCCTGACCGCCAAGTACCCCGGCAACTTCACCGCCACCATCGAGGAGTGGCAGGCCGAGCA
CGACGCCATCATGAGGCACATCCTGGAGAGGCCCGACCCCACCGACGTGTTCCAGAACAAGGCC
AACGTGTGCTGGGCCAAGGCCCTGGTGCCCGTGCTGAAGACCGCCGGCATCGACATGACCACCG
AGCAGTGGAACACCGTGGACTACTTCGAGACAGACAAGGCCCACAGCGCCGAGATCGTGCTGAA
CCAGCTGTGCGTGAGGTTCTTCGGCCTGGACCTGGACAGCGGCCTGTTCAGCGCCCCTACCGTG
CCCCTGAGCATCAGGAACAACCACTGGGACAACAGCCCCAGCCCCAACATGTACGGCCTGAACAA
GGAGGTGGTGAGGCAGCTGAGCAGGCGGTACCCTCAGCTGCCCAGGGCCGTGGCCACCGGCAG
GGTGTACGACATGAACACCGGCACCCTGAGGAACTACGACCCCAGGATCAACCTGGTGCCCGTG
AACAGGCGGCTGCCACACGCCCTGGTGCTGCACCACAACGAGCACCCTCAGAGCGACTTCAGCA
GCTTCGTGAGCAAGCTGAAGGGCAGGACCGTGCTGGTGGTGGGCGAGAAGCTGAGCGTGCCCG
GCAAGATGGTGGACTGGCTGAGCGACAGGCCCGAGGCCACCTTCCGGGCCAGGCTGGACCTGG
GCATCCCCGGCGACGTGCCCAAGTACGACATCATCTTCGTGAACGTGAGGACCCCTTACAAGTAC
CACCACTACCAGCAGTGCGAGGACCACGCCATCAAGCTGAGCATGCTGACCAAGAAGGCCTGCC
TGCACCTGAACCCCGGCGGCACCTGCGTGAGCATCGGCTACGGCTACGCCGACAGGGCCAGCG
AGAGCATCATCGGCGCCATCGCCAGGCTGTTCAAGTTCAGCAGGGTGTGCAAGCCCAAGAGCAG
CCTGGAGGAGACAGAGGTGCTGTTCGTGTTCATCGGCTACGACCGGAAGGCCAGGACCCACAAC
CCCTACAAGCTGAGCAGCACCCTGACCAACATCTACACCGGCAGCAGGCTGCACGAGGCCGGCT
GCGCCCCTAGCTACCACGTGGTGAGGGGCGACATCGCCACCGCCACCGAGGGCGTGATCATCAA
CGCCGCCAACAGCAAGGGCCAGCCCGGCGGCGGGGTGTGCGGCGCCCTGTATAAGAAGTTCCC
CGAGAGCTTCGACCTGCAGCCCATCGAGGTGGGCAAGGCCAGGCTGGTGAAGGGCGCCGCCAA
GCACATCATCCACGCCGTGGGCCCCAACTTCAACAAGGTGAGCGAGGTGGAGGGCGACAAGCAG
CTGGCCGAGGCCTACGAGAGCATCGCCAAGATCGTGAACGACAACAACTACAAGAGCGTGGCCA
TCCCTCTGCTGAGCACCGGCATCTTCAGCGGCAACAAGGACAGGCTGACCCAGAGCCTGAACCA
CCTGCTGACCGCCCTGGACACCACCGACGCCGACGTGGCCATCTACTGCAGGGACAAGAAGTGG
GAGATGACCCTGAAGGAGGCCGTGGCCAGGCGGGAGGCCGTGGAGGAGATCTGCATCAGCGAC
GACAGCAGCGTGACGGAGCCCGACGCCGAGCTGGTGAGGGTGCACCCCAAGAGCAGCCTGGCC
GGCAGGAAGGGCTACAGCACCAGCGACGGCAAGACCTTCAGCTACCTGGAGGGCACCAAGTTCC
ACCAGGCCGCCAAGGACATCGCCGAGATCAACGCCATGTGGCCCGTGGCCACCGAGGCCAACGA
GCAGGTGTGCATGTATATCCTGGGCGAGAGCATGAGCAGCATCAGGAGCAAGTGCCCCGTGGAG
GAGAGCGAGGCCAGCACCCCTCCCAGCACCCTGCCCTGCCTGTGCATCCACGCCATGACCCCTG
AGAGGGTGCAGCGGCTGAAGGCCAGCAGGCCCGAGCAGATCACCGTGTGCAGCAGCTTCCCTCT
GCCCAAGTACCGGATCACCGGCGTGCAGAAGATCCAGTGCAGCCAGCCCATCCTGTTCAGCCCC
AAGGTGCCCGCCTACATCCACCCCAGGAAGTACCTGGTGGAGACACCCCCCGTGGACGAGACAC
CCGAGCCCAGCGCCGAGAACCAGAGCACCGAGGGCACCCCTGAGCAGCCTCCCCTGATCACCG
AGGACGAGACAAGGACCAGGACGCCCGAGCCCATCATCATTGAGGAGGAAGAGGAGGACAGCAT
CAGCCTGCTGAGCGACGGCCCCACCCACCAGGTGCTGCAGGTGGAGGCCGACATCCACGGCCC
TCCCAGCGTGAGCAGCTCCAGCTGGAGCATCCCTCACGCCAGCGACTTCGACGTGGACAGCCTG
AGCATCCTGGACACCCTGGAGGGCGCCAGCGTGACCAGCGGCGCCACCAGCGCCGAGACAAAC
AGCTACTTCGCCAAGAGCATGGAGTTCCTGGCCAGGCCCGTGCCCGCCCCTAGGACCGTGTTCA
GGAACCCTCCCCACCCCGCCCCTAGGACCAGGACCCCTAGCCTGGCCCCTAGCAGGGCCTGCAG
CAGGACCAGCCTGGTGAGCACCCCTCCCGGCGTGAACCGGGTGATCACCAGGGAGGAGCTGGA
GGCCCTGACCCCTAGCAGGACCCCTAGCAGGAGCGTGAGCAGGACCAGCCTGGTGAGCAACCCT
CCCGGCGTGAACCGGGTGATCACCAGGGAGGAGTTCGAGGCCTTCGTGGCCCAGCAGCAAAGG
CGGTTCGACGCCGGCGCCTACATCTTCAGCAGCGACACCGGCCAGGGCCACCTGCAGCAGAAGT
CCGTGAGGCAGACCGTGCTGAGCGAGGTGGTCCTGGAGAGGACGGAGCTGGAGATCAGCTACG
CCCCTAGGCTGGACCAGGAGAAGGAGGAGCTGCTGAGGAAGAAGCTGCAGCTGAACCCCACCCC
TGCCAACAGGAGCAGGTACCAGAGCAGGAAGGTGGAGAACATGAAGGCCATCACCGCCAGGCGG
ATCCTGCAGGGCCTGGGCCACTACCTGAAGGCCGAGGGCAAGGTGGAGTGCTACAGGACCCTGC
ACCCCGTGCCCCTGTACTCCAGCTCCGTGAACAGGGCCTTCAGCAGCCCCAAGGTGGCCGTGGA
GGCCTGCAACGCCATGCTGAAGGAGAACTTCCCCACCGTGGCCAGCTACTGCATCATCCCCGAGT
ACGACGCCTACCTGGACATGGTGGACGGCGCCAGCTGCTGCCTGGACACCGCCAGCTTCTGCCC
CGCCAAGCTGAGGAGCTTCCCCAAGAAGCACAGCTACCTGGAGCCCACCATCAGGAGCGCCGTG
CCCAGCGCCATCCAGAACACCCTGCAGAACGTGCTGGCCGCCGCTACCAAGAGGAACTGCAACG
TGACCCAGATGAGGGAGCTGCCCGTGCTGGACAGCGCCGCCTTCAACGTGGAGTGCTTCAAGAA
GTACGCCTGCAACAACGAGTACTGGGAGACATTCAAGGAGAACCCCATCAGGCTGACCGAGGAG
AACGTGGTGAACTACATCACCAAGCTGAAGGGCCCCAAGGCCGCCGCTCTGTTCGCCAAGACCCA
CAACCTGAACATGCTCCAGGACATCCCTATGGACAGGTTCGTGATGGACCTGAAGAGGGACGTGA
AGGTGACCCCTGGCACCAAGCACACCGAGGAGAGGCCCAAGGTGCAGGTGATCCAGGCCGCCG
ACCCTCTGGCCACCGCCTACCTGTGCGGCATCCACAGGGAGCTGGTGAGGCGGCTGAACGCCGT
CCTGCTGCCCAACATCCACACCCTGTTCGACATGAGCGCCGAGGACTTCGACGCCATCATCGCCG
AGCACTTCCAGCCCGGCGACTGCGTGCTGGAGACAGACATCGCCAGCTTCGACAAGAGCGAGGA
CGACGCTATGGCCCTGACCGCCCTGATGATCCTGGAGGACCTGGGCGTGGACGCCGAGCTGCTG
ACCCTGATCGAGGCCGCCTTCGGCGAGATCAGCAGCATCCACCTGCCCACCAAGACCAAGTTCAA
GTTCGGCGCCATGATGAAGTCCGGCATGTTCCTGACCCTGTTCGTGAACACCGTGATCAACATCG
TGATCGCCAGCAGGGTGCTGCGGGAGAGGCTGACCGGCAGCCCCTGCGCCGCCTTCATCGGCG
ACGACAACATCGTGAAGGGCGTGAAGTCCGACAAGCTGATGGCCGACAGGTGCGCCACCTGGCT
GAACATGGAGGTGAAGATCATCGACGCCGTGGTGGGCGAGAAGGCCCCTTACTTCTGCGGCGGC
TTCATCCTGTGCGACAGCGTGACCGGCACCGCCTGCAGGGTGGCCGACCCTCTGAAGAGGCTGT
TCAAGCTGGGCAAGCCCCTGGCCGCCGACGACGAGCACGACGACGATAGGCGGAGGGCCCTGC
ACGAGGAGAGCACCAGGTGGAACCGGGTGGGCATCCTGAGCGAGCTGTGCAAGGCCGTGGAGA
GCAGGTACGAGACAGTGGGCACCAGCATCATCGTGATGGCCATGACCACCCTGGCCAGCAGCGT
CAAGTCCTTCAGCTACCTGAGGGGGGCCCCTATAACTCTCTACGGCTAACCTGAATGGACTACG
ACATAGTCTAGTCCGCCAAGGCCGCCACCATGAATCCTAACCAGAAGATCATCACCATCG
GCAGCGTGAGCCTGACCATCAGCACCATCTGCTTCTTCATGCAGATCGCCATCCTGAT
CACCACCGTGACCCTGCACTTCAAGCAGTACGAGTTCAACAGCCCTCCCAACAACCAG
GTGATGCTGTGCGAGCCCACCATCATCGAGAGGAACATCACCGAGATCGTGTACCTGA
CCAACACCACCATCGAGAAGGAGATCTGCCCCAAGCCCGCCGAGTACAGGAACTGGA
GCAAGCCCCAGTGCGGCATCACCGGCTTCGCCCCTTTCAGCAAGGACAACAGCATCAG
GCTGAGCGCCGGCGGCGACATCTGGGTGACCAGGGAGCCCTACGTGAGCTGCGACCT
GGACAAGTGCTACCAGTTCGCCCTGGGCCAGGGCACCACCCTGAACAACGTGCACAG
CAACAACACCGTGAGGGACAGGACCCCTTACCGGACCCTGCTGATGAACGAGCTGGG
CGTGCCCTTCCACCTGGGCACCAAGCAGGTGTGCATCGCCTGGAGCAGCTCCAGCTGC
CACGACGGCAAGGCCTGGCTGCACGTGTGCATCACCGGCGACGACAAGAACGCCACC
GCCAGCTTCATCTACAACGGCAGGCTGGTGGACAGCGTGGTGAGCTGGAGCAACGAC
ATCCTGAGGACCCAGGAGAGCGAGTGCGTGTGCATCAACGGCACCTGCACCGTGGTG
ATGACCGACGGCAACGCCACCGGCAAGGCCGACACCAAGATCCTGTTCATCGAGGAG
GGCAAGATCGTGCACACCAGCAAGCTGAGCGGCAGCGCCCAGCACGTGGAGGAGTGC
AGCTGCTACCCCAGGTACCCCGGCGTGAGGTGCGTGTGCAGGGACAACTGGAAGGGC
AGCAACCGGCCCATCATCGACATCAACATCAAGGACCACAGCATCGTCAGCAGGTACG
TGTGCAGCGGCCTGGTGGGCGACACCCCTAGAAAGAGCGACAGCAGCTCCAGCAGCC
ACTGCCTGAACCCCAACAACGAGAAGGGCGACCACGGCGTGAAGGGCTGGGCCTTCG
ACGACGGCAACGACGTGTGGATGGGCAGGACCATCAACGAGACAAGCAGGCTGGGCT
ACGAGACATTCAAGGTGGTGGAGGGCTGGAGCAACCCCAAGAGCAAGCTGCAGATCA
ACAGGCAGGTGATCGTGGACAGGGGCGACAGGAGCGGCTACAGCGGCATCTTCAGCG
TGGAGGGCAAGAGCTGCATCAACCGGTGCTTCTACGTGGAGCTGATCAGGGGCAGGA
AGGAGGAGACAGAGGTGCTGTGGACCAGCAACAGCATCGTGGTGTTCTGCGGCACCA
GCGGCACCTACGGCACCGGCAGCTGGCCCGACGGCGCCAACCTGAGCCTGATGCACA
TCTGATAGTAAACTCGAAGCAGTGTTAAATCATTCAGCTACCTGAGAGGGGCCCCTATAAC
TCTCTACGGCTAACCTGAATGGACTACGACATAGTCTAGTCCGCCAAGGCCGCCACCATGA
AGACCATCATCGCCCTGAGCAACATCCTGTGCCTGGTGTTCGCCCAGAAGATCCCCGG
CAATGACAACAGCACCGCCACCCTGTGCCTGGGCCACCACGCCGTGCCCAACGGCACC
ATCGTGAAGACCATCACCAACGACAGGATCGAGGTGACCAACGCCACCGAGCTGGTG
CAGAACAGCAGCATCGGCGAGATCTGCGGCAGCCCTCACCAGATCCTGGACGGCGGC
AACTGCACCCTGATCGACGCCCTGCTGGGCGACCCTCAGTGCGACGGCTTCCAGAACA
AGGAGTGGGACCTGTTCGTGGAGAGGAGCAGGGCCAACAGCAACTGCTACCCCTACG
ACGTGCCCGACTACGCCAGCCTGAGGAGCCTGGTGGCCAGCAGCGGCACCCTGGAGT
TCAAGAACGAGAGCTTCAACTGGACCGGCGTGAAGCAGAACGGCACCAGCAGCGCCT
GCATCAGGGGCAGCAGCTCCAGCTTCTTCAGCCGGCTGAATTGGCTCACCAGCCTGAA
CAACATCTACCCCGCCCAGAACGTGACCATGCCCAACAAGGAGCAGTTCGACAAGCTG
TATATCTGGGGCGTGCACCACCCCGACACCGACAAGAACCAGATCAGCCTGTTCGCCC
AGAGCAGCGGCAGGATCACCGTGAGCACCAAGAGGAGCCAGCAGGCCGTGATCCCCA
ACATCGGCAGCAGGCCCAGGATCAGGGGCATCCCCAGCAGGATCAGCATCTACTGGA
CCATCGTGAAGCCCGGCGACATCCTGCTGATCAACAGCACCGGCAACCTGATCGCCCC
TAGGGGCTACTTCAAGATCAGGAGCGGCAAGAGCAGCATCATGAGGAGCGACGCCCC
TATCGGCAAGTGCAAGAGCGAGTGCATCACCCCTAACGGCAGCATCCCCAACGACAAG
CCCTTCCAGAACGTGAACAGGATCACCTACGGCGCCTGCCCCAGGTACGTGAAGCAGA
GCACCCTGAAGCTGGCCACCGGCATGAGGAACGTGCCCGAGAAGCAGACCAGGGGCA
TCTTCGGCGCCATCGCCGGCTTCATCGAGAACGGCTGGGAGGGCATGGTGGACGGCT
GGTACGGCTTCAGGCACCAGAACAGCGAGGGCAGGGGCCAGGCCGCCGACCTGAAGT
CCACCCAGGCCGCCATCGACCAGATCAACGGCAAGCTGAACAGGCTGATCGGCAAGA
CCAACGAGAAGTTCCACCAGATCGAGAAGGAGTTCAGCGAGGTGGAGGGCAGGGTGC
AGGACCTGGAGAAGTACGTGGAGGACACCAAGATCGACCTGTGGAGCTACAACGCCG
AGCTGCTGGTGGCCCTGGAGAACCAGCACACCATCGACCTGACCGACAGCGAGATGA
ACAAGCTGTTCGAGAAGACCAAGAAGCAGCTGAGGGAGAACGCCGAGGACATGGGCA
ACGGCTGCTTCAAGATCTACCACAAGTGCGACAACGCCTGCATCGGCAGCATCAGGAA
CGAGACATATGACCACAACGTGTACCGGGACGAGGCCCTGAACAACCGGTTCCAGATC
AAGGGCGTGGAGCTGAAGTCCGGCTACAAGGACTGGATCCTGTGGATCAGCTTCGCC
ATGAGCTGCTTCCTGCTGTGCATCGCCCTGCTGGGCTTCATCATGTGGGCCTGCCAGA
AGGGCAACATCAGGTGCAACATCTGCATCTGATAGTAAACTCGAGTATGTTACGTGCAA
AGGTGATTGTCACCCCCCGAAAGACCATATTGTGACACACCCTCAGTATCACGCCCAAACAT
TTACAGCCGCGGTGTCAAAAACCGCGTGGACGTGGTTAACATCCCTGCTGGGAGGATCAGC
CGTAATTATTATAATTGGCTTGGTGCTGGCTACTATTGTGGCCATGTACGTGCTGACCAACC
AGAAACATAATTGAATACAGCAGCAATTGGCAAGCTGCTTACATAGAACTCGCGGCGATTG
GCATGCCGCCTTAAAATTTTTATTTTATTTTTTCTTTTCTTTTCCGAATCGGATTTTGTTTTTA
ATATTTC
SEQ ID NO: 3 (B Victoria bicistronic construct; self-replicating RNA for
expression of NA/HA from B Victoria influenza strain)
ATGGGCGGCGCATGAGAGAAGCCCAGACCAATTACCTACCCAAAATGGAGAAAGTTCACGTT
GACATCGAGGAAGACAGCCCATTCCTCAGAGCTTTGCAGCGGAGCTTCCCGCAGTTTGAGGTAGA
AGCCAAGCAGGTCACTGATAATGACCATGCTAATGCCAGAGCGTTTTCGCATCTGGCTTCAAAACT
GATCGAAACGGAGGTGGACCCATCCGACACGATCCTTGACATTGGAAGTGCGCCCGCCCGCAGA
ATGTATTCTAAGCACAAGTATCATTGTATCTGTCCGATGAGATGTGCGGAAGATCCGGACAGATTG
TATAAGTATGCAACTAAGCTGAAGAAAAACTGTAAGGAAATAACTGATAAGGAATTGGACAAGAAAA
TGAAGGAGCTGGCCGCCGTCATGAGCGACCCTGACCTGGAAACTGAGACTATGTGCCTCCACGA
CGACGAGTCGTGTCGCTACGAAGGGCAAGTCGCTGTTTACCAGGATGTATACGCCGTCGACGGC
CCCACCAGCCTGTACCACCAGGCCAACAAGGGCGTGAGGGTGGCCTACTGGATCGGCTTCGACA
CCACACCCTTCATGTTCAAGAACCTGGCCGGCGCCTACCCCAGCTACAGCACCAACTGGGCCGAC
GAGACAGTGCTGACCGCCAGGAACATCGGCCTGTGCAGCAGCGACGTGATGGAGAGGAGCCGG
AGGGGCATGAGCATCCTGAGGAAGAAGTACCTGAAGCCCAGCAACAACGTGCTGTTCAGCGTGG
GCAGCACCATCTACCACGAGAAGAGGGACCTGCTGAGGAGCTGGCACCTGCCCAGCGTGTTCCA
CCTGAGGGGCAAGCAGAACTACACCTGCAGGTGCGAGACAATCGTGAGCTGCGACGGCTACGTG
GTGAAGAGGATCGCCATCAGCCCCGGCCTGTACGGCAAGCCCAGCGGCTACGCCGCCACCATGC
ACAGGGAGGGCTTCCTGTGCTGCAAGGTGACCGACACCCTGAACGGCGAGAGGGTGAGCTTCCC
CGTGTGCACCTACGTGCCCGCCACCCTGTGCGACCAGATGACCGGCATCCTGGCCACCGACGTG
AGCGCCGACGACGCCCAGAAGCTGCTGGTGGGCCTGAACCAGAGGATCGTGGTGAACGGCAGG
ACCCAGAGGAACACCAACACCATGAAGAACTACCTGCTGCCCGTGGTGGCCCAGGCCTTCGCCA
GGTGGGCCAAGGAGTACAAGGAGGACCAGGAGGACGAGAGGCCCCTGGGCCTGAGGGACCGAC
AGCTGGTGATGGGCTGCTGCTGGGCCTTCAGGCGGCACAAGATCACCAGCATCTACAAGAGGCC
CGACACCCAGACCATCATCAAGGTGAACAGCGACTTCCACAGCTTCGTGCTGCCCAGGATCGGCA
GCAACACCCTGGAGATCGGCCTGAGGACCCGGATCAGGAAGATGCTGGAGGAGCACAAGGAGCC
CAGCCCTCTGATCACCGCCGAGGACGTGCAGGAGGCCAAGTGCGCCGCCGACGAGGCCAAGGA
GGTGAGGGAGGCCGAGGAGCTGAGGGCCGCCCTGCCTCCCCTGGCCGCCGACGTGGAGGAGC
CCACCCTGGAGGCCGACGTGGACCTGATGCTGCAGGAGGCCGGCGCCGGCAGCGTGGAGACAC
CCAGGGGCCTGATCAAGGTGACCAGCTACGACGGCGAGGACAAGATCGGCAGCTACGCCGTGCT
CAGCCCTCAGGCCGTGCTGAAGTCCGAGAAGCTGAGCTGCATCCACCCTCTGGCCGAGCAGGTG
ATCGTGATCACCCACAGCGGCAGGAAGGGCAGGTACGCCGTGGAGCCCTACCACGGCAAGGTG
GTGGTCCCCGAGGGCCACGCCATCCCCGTGCAGGACTTCCAGGCCCTGAGCGAGAGCGCCACC
ATCGTGTATAACGAGAGGGAGTTCGTGAACAGGTACCTGCACCACATCGCCACCCACGGCGGCG
CCCTGAACACCGACGAGGAGTACTACAAGACCGTGAAGCCCAGCGAGCACGACGGCGAGTACCT
GTACGACATCGACAGGAAGCAGTGCGTGAAGAAGGAGCTGGTGACCGGCCTGGGCCTGACCGG
CGAGCTGGTGGACCCTCCCTTCCACGAGTTCGCCTACGAGAGCCTGAGGACCAGGCCCGCCGCT
CCCTACCAGGTGCCCACCATCGGCGTGTACGGCGTGCCCGGCAGCGGCAAGAGCGGCATCATCA
AGAGCGCCGTGACCAAGAAGGACCTGGTGGTGAGCGCCAAGAAGGAGAACTGCGCCGAGATCAT
CAGGGACGTGAAGAAGATGAAGGGCCTGGACGTGAACGCCAGGACCGTGGACAGCGTGCTCCTG
AACGGCTGCAAGCACCCCGTGGAGACACTGTATATCGACGAGGCCTTCGCCTGCCACGCCGGCA
CCCTGAGGGCCCTGATCGCCATCATCAGGCCCAAGAAGGCCGTGCTGTGCGGCGACCCCAAGCA
GTGCGGCTTCTTCAACATGATGTGCCTGAAGGTGCACTTCAACCACGAGATCTGCACCCAGGTGTT
CCACAAGAGCATCAGCAGGCGGTGCACCAAGAGCGTGACCAGCGTGGTGAGCACCCTGTTCTAC
GACAAGAAGATGAGGACCACCAACCCCAAGGAGACAAAGATCGTGATCGACACCACCGGCAGCA
CCAAGCCCAAGCAGGACGACCTGATCCTGACCTGCTTCAGGGGCTGGGTGAAGCAGCTGCAGAT
CGACTACAAGGGCAACGAGATCATGACCGCCGCCGCTAGCCAGGGCCTGACCAGGAAGGGCGT
GTACGCCGTGAGGTACAAGGTGAACGAGAATCCCCTGTACGCCCCTACCAGCGAGCACGTGAAC
GTCCTGCTGACCAGGACCGAGGACAGGATCGTGTGGAAGACCCTGGCCGGCGACCCCTGGATCA
AGACCCTGACCGCCAAGTACCCCGGCAACTTCACCGCCACCATCGAGGAGTGGCAGGCCGAGCA
CGACGCCATCATGAGGCACATCCTGGAGAGGCCCGACCCCACCGACGTGTTCCAGAACAAGGCC
AACGTGTGCTGGGCCAAGGCCCTGGTGCCCGTGCTGAAGACCGCCGGCATCGACATGACCACCG
AGCAGTGGAACACCGTGGACTACTTCGAGACAGACAAGGCCCACAGCGCCGAGATCGTGCTGAA
CCAGCTGTGCGTGAGGTTCTTCGGCCTGGACCTGGACAGCGGCCTGTTCAGCGCCCCTACCGTG
CCCCTGAGCATCAGGAACAACCACTGGGACAACAGCCCCAGCCCCAACATGTACGGCCTGAACAA
GGAGGTGGTGAGGCAGCTGAGCAGGCGGTACCCTCAGCTGCCCAGGGCCGTGGCCACCGGCAG
GGTGTACGACATGAACACCGGCACCCTGAGGAACTACGACCCCAGGATCAACCTGGTGCCCGTG
AACAGGCGGCTGCCACACGCCCTGGTGCTGCACCACAACGAGCACCCTCAGAGCGACTTCAGCA
GCTTCGTGAGCAAGCTGAAGGGCAGGACCGTGCTGGTGGTGGGCGAGAAGCTGAGCGTGCCCG
GCAAGATGGTGGACTGGCTGAGCGACAGGCCCGAGGCCACCTTCCGGGCCAGGCTGGACCTGG
GCATCCCCGGCGACGTGCCCAAGTACGACATCATCTTCGTGAACGTGAGGACCCCTTACAAGTAC
CACCACTACCAGCAGTGCGAGGACCACGCCATCAAGCTGAGCATGCTGACCAAGAAGGCCTGCC
TGCACCTGAACCCCGGCGGCACCTGCGTGAGCATCGGCTACGGCTACGCCGACAGGGCCAGCG
AGAGCATCATCGGCGCCATCGCCAGGCTGTTCAAGTTCAGCAGGGTGTGCAAGCCCAAGAGCAG
CCTGGAGGAGACAGAGGTGCTGTTCGTGTTCATCGGCTACGACCGGAAGGCCAGGACCCACAAC
CCCTACAAGCTGAGCAGCACCCTGACCAACATCTACACCGGCAGCAGGCTGCACGAGGCCGGCT
GCGCCCCTAGCTACCACGTGGTGAGGGGCGACATCGCCACCGCCACCGAGGGCGTGATCATCAA
CGCCGCCAACAGCAAGGGCCAGCCCGGCGGCGGGGTGTGCGGCGCCCTGTATAAGAAGTTCCC
CGAGAGCTTCGACCTGCAGCCCATCGAGGTGGGCAAGGCCAGGCTGGTGAAGGGCGCCGCCAA
GCACATCATCCACGCCGTGGGCCCCAACTTCAACAAGGTGAGCGAGGTGGAGGGCGACAAGCAG
CTGGCCGAGGCCTACGAGAGCATCGCCAAGATCGTGAACGACAACAACTACAAGAGCGTGGCCA
TCCCTCTGCTGAGCACCGGCATCTTCAGCGGCAACAAGGACAGGCTGACCCAGAGCCTGAACCA
CCTGCTGACCGCCCTGGACACCACCGACGCCGACGTGGCCATCTACTGCAGGGACAAGAAGTGG
GAGATGACCCTGAAGGAGGCCGTGGCCAGGCGGGAGGCCGTGGAGGAGATCTGCATCAGCGAC
GACAGCAGCGTGACGGAGCCCGACGCCGAGCTGGTGAGGGTGCACCCCAAGAGCAGCCTGGCC
GGCAGGAAGGGCTACAGCACCAGCGACGGCAAGACCTTCAGCTACCTGGAGGGCACCAAGTTCC
ACCAGGCCGCCAAGGACATCGCCGAGATCAACGCCATGTGGCCCGTGGCCACCGAGGCCAACGA
GCAGGTGTGCATGTATATCCTGGGCGAGAGCATGAGCAGCATCAGGAGCAAGTGCCCCGTGGAG
GAGAGCGAGGCCAGCACCCCTCCCAGCACCCTGCCCTGCCTGTGCATCCACGCCATGACCCCTG
AGAGGGTGCAGCGGCTGAAGGCCAGCAGGCCCGAGCAGATCACCGTGTGCAGCAGCTTCCCTCT
GCCCAAGTACCGGATCACCGGCGTGCAGAAGATCCAGTGCAGCCAGCCCATCCTGTTCAGCCCC
AAGGTGCCCGCCTACATCCACCCCAGGAAGTACCTGGTGGAGACACCCCCCGTGGACGAGACAC
CCGAGCCCAGCGCCGAGAACCAGAGCACCGAGGGCACCCCTGAGCAGCCTCCCCTGATCACCG
AGGACGAGACAAGGACCAGGACGCCCGAGCCCATCATCATTGAGGAGGAAGAGGAGGACAGCAT
CAGCCTGCTGAGCGACGGCCCCACCCACCAGGTGCTGCAGGTGGAGGCCGACATCCACGGCCC
TCCCAGCGTGAGCAGCTCCAGCTGGAGCATCCCTCACGCCAGCGACTTCGACGTGGACAGCCTG
AGCATCCTGGACACCCTGGAGGGCGCCAGCGTGACCAGCGGCGCCACCAGCGCCGAGACAAAC
AGCTACTTCGCCAAGAGCATGGAGTTCCTGGCCAGGCCCGTGCCCGCCCCTAGGACCGTGTTCA
GGAACCCTCCCCACCCCGCCCCTAGGACCAGGACCCCTAGCCTGGCCCCTAGCAGGGCCTGCAG
CAGGACCAGCCTGGTGAGCACCCCTCCCGGCGTGAACCGGGTGATCACCAGGGAGGAGCTGGA
GGCCCTGACCCCTAGCAGGACCCCTAGCAGGAGCGTGAGCAGGACCAGCCTGGTGAGCAACCCT
CCCGGCGTGAACCGGGTGATCACCAGGGAGGAGTTCGAGGCCTTCGTGGCCCAGCAGCAAAGG
CGGTTCGACGCCGGCGCCTACATCTTCAGCAGCGACACCGGCCAGGGCCACCTGCAGCAGAAGT
CCGTGAGGCAGACCGTGCTGAGCGAGGTGGTCCTGGAGAGGACGGAGCTGGAGATCAGCTACG
CCCCTAGGCTGGACCAGGAGAAGGAGGAGCTGCTGAGGAAGAAGCTGCAGCTGAACCCCACCCC
TGCCAACAGGAGCAGGTACCAGAGCAGGAAGGTGGAGAACATGAAGGCCATCACCGCCAGGCGG
ATCCTGCAGGGCCTGGGCCACTACCTGAAGGCCGAGGGCAAGGTGGAGTGCTACAGGACCCTGC
ACCCCGTGCCCCTGTACTCCAGCTCCGTGAACAGGGCCTTCAGCAGCCCCAAGGTGGCCGTGGA
GGCCTGCAACGCCATGCTGAAGGAGAACTTCCCCACCGTGGCCAGCTACTGCATCATCCCCGAGT
ACGACGCCTACCTGGACATGGTGGACGGCGCCAGCTGCTGCCTGGACACCGCCAGCTTCTGCCC
CGCCAAGCTGAGGAGCTTCCCCAAGAAGCACAGCTACCTGGAGCCCACCATCAGGAGCGCCGTG
CCCAGCGCCATCCAGAACACCCTGCAGAACGTGCTGGCCGCCGCTACCAAGAGGAACTGCAACG
TGACCCAGATGAGGGAGCTGCCCGTGCTGGACAGCGCCGCCTTCAACGTGGAGTGCTTCAAGAA
GTACGCCTGCAACAACGAGTACTGGGAGACATTCAAGGAGAACCCCATCAGGCTGACCGAGGAG
AACGTGGTGAACTACATCACCAAGCTGAAGGGCCCCAAGGCCGCCGCTCTGTTCGCCAAGACCCA
CAACCTGAACATGCTCCAGGACATCCCTATGGACAGGTTCGTGATGGACCTGAAGAGGGACGTGA
AGGTGACCCCTGGCACCAAGCACACCGAGGAGAGGCCCAAGGTGCAGGTGATCCAGGCCGCCG
ACCCTCTGGCCACCGCCTACCTGTGCGGCATCCACAGGGAGCTGGTGAGGCGGCTGAACGCCGT
CCTGCTGCCCAACATCCACACCCTGTTCGACATGAGCGCCGAGGACTTCGACGCCATCATCGCCG
AGCACTTCCAGCCCGGCGACTGCGTGCTGGAGACAGACATCGCCAGCTTCGACAAGAGCGAGGA
CGACGCTATGGCCCTGACCGCCCTGATGATCCTGGAGGACCTGGGCGTGGACGCCGAGCTGCTG
ACCCTGATCGAGGCCGCCTTCGGCGAGATCAGCAGCATCCACCTGCCCACCAAGACCAAGTTCAA
GTTCGGCGCCATGATGAAGTCCGGCATGTTCCTGACCCTGTTCGTGAACACCGTGATCAACATCG
TGATCGCCAGCAGGGTGCTGCGGGAGAGGCTGACCGGCAGCCCCTGCGCCGCCTTCATCGGCG
ACGACAACATCGTGAAGGGCGTGAAGTCCGACAAGCTGATGGCCGACAGGTGCGCCACCTGGCT
GAACATGGAGGTGAAGATCATCGACGCCGTGGTGGGCGAGAAGGCCCCTTACTTCTGCGGCGGC
TTCATCCTGTGCGACAGCGTGACCGGCACCGCCTGCAGGGTGGCCGACCCTCTGAAGAGGCTGT
TCAAGCTGGGCAAGCCCCTGGCCGCCGACGACGAGCACGACGACGATAGGCGGAGGGCCCTGC
ACGAGGAGAGCACCAGGTGGAACCGGGTGGGCATCCTGAGCGAGCTGTGCAAGGCCGTGGAGA
GCAGGTACGAGACAGTGGGCACCAGCATCATCGTGATGGCCATGACCACCCTGGCCAGCAGCGT
CAAGTCCTTCAGCTACCTGAGGGGGGCCCCTATAACTCTCTACGGCTAACCTGAATGGACTACG
ACATAGTCTAGTCCGCCAAGGCCGCCACCATGAACCCCAACCAGAAGATCATCACCATCG
GCAGCATCTGCATGACCATCGGCACCGCCAACCTGATCCTGCAGATCGGCAACATCAT
CAGCATCTGGGTGAGCCACAGCATCCAGATCGGCAACCAGAGCCAGATCGAGACATG
CAACAAGAGCGTGATCACCTACGAGAACAACACCTGGGTGAACCAGACCTTCGTGAAC
ATCAGCAACACCAACAGCGCCGCCAGGCAGAGCGTGGCCAGCGTGAAGCTGGCCGGC
AACAGCAGCCTGTGCCCCGTGAGCGGCTGGGCCATCTACAGCAAGGACAACAGCGTG
AGGATCGGCAGCAAGGGCGACGTGTTCGTGATCAGGGAGCCCTTCATCAGCTGCAGC
CCTCTGGAGTGCAGGACCTTCTTCCTGACCCAGGGCGCCCTGCTGAACGACAAGCACA
GCAACGGCACCATCAAGGACAGGAGCCCCTACAGGACCCTGATGAGCTGCCCCATCG
GCGAGGTGCCCAGCCCCTACAACAGCAGGTTCGAGAGCGTGGCCTGGAGCGCCAGCG
CCTGCCACGACGGCACCAACTGGCTGACCATCGGCATCAGCGGCCCCGACAGCGGCG
CCGTGGCCGTGCTGAAGTACAACGGCATCATCACCGACACCATCAAGAGCTGGAGGAA
CAAGATCCTGAGGACCCAGGAGAGCGAGTGCGCCTGCGTGAACGGCAGCTGCTTCAC
CATCATGACCGACGGCCCCAGCGACGGCCAGGCCAGCTACAAGATCTTCAGGATCGA
GAAGGGCAAGATCATCAAGAGCGTGGAGATGAAGGCCCCTAACTACCACTACGAGGA
GTGCAGCTGCTACCCCGACAGCAGCGAGATCACCTGCGTGTGCAGGGACAACTGGCA
CGGCAGCAACAGGCCCTGGGTGAGCTTCAACCAGAACCTGGAGTACCAGATGGGCTA
CATCTGCAGCGGCGTGTTCGGCGACAACCCCAGGCCCAACGACAAGACCGGCAGCTG
CGGCCCCGTGAGCAGCAACGGCGCCAACGGCGTGAAGGGCTTCAGCTTCAAGTACGG
CAACGGCGTGTGGATCGGCAGGACCAAGAGCATCAGCAGCAGGAAGGGCTTCGAGAT
GATCTGGGACCCCAACGGCTGGACCGGCACCGACAACAAGTTCAGCAAGAAGCAGGA
CATCGTGGGCATCAACGAGTGGAGCGGCTACAGCGGCAGCTTCGTGCAGCACCCCGA
GCTGACCGGCCTGAACTGCATCAGGCCCTGCTTCTGGGTGGAGCTGATCAGGGGCAG
GCCCGAGGAGAACACCATCTGGACCAGCGGCAGCAGCATCAGCTTCTGCGGCGTGGA
CAGCGACATCGTGGGCTGGAGCTGGCCCGACGGCGCCGAGCTGCCCTTCACCATCGA
CAAGTGATAGTAAACTCGAAGCAGTGTTAAATCATTCAGCTACCTGAGAGGGGCCCCTATA
ACTCTCTACGGCTAACCTGAATGGACTACGACATAGTCTAGTCCGCCAAGGCCGCCACCAT
GAAGGCCATCCTGGTGGTGATGCTGTATACCTTCACCACCGCTAACGCCGACACCCTG
TGCATCGGCTACCACGCCAACAACAGCACCGACACCGTGGACACCGTGCTGGAGAAG
AACGTGACCGTGACCCACAGCGTGAACCTGCTGGAGGACAAGCACAACGGCAAGCTG
TGCAAGCTGAGGGGCGTGGCCCCTCTGCACCTGGGCAAGTGCAACATCGCCGGCTGG
ATCCTGGGCAACCCCGAGTGCGAGAGCCTGAGCACCGCCAGGAGCTGGAGCTACATC
GTGGAGACAAGCAACAGCGACAACGGCACCTGCTACCCCGGCGACTTCATCAACTACG
AGGAGCTGAGGGAGCAGCTGAGCAGCGTGAGCAGCTTCGAGAGGTTCGAGATCTTCC
CCAAGACCAGCAGCTGGCCCAACCACGACAGCGACAACGGCGTGACCGCCGCCTGCC
CTCACGCCGGCGCCAAGAGCTTCTACAAGAACCTGATCTGGCTGGTGAAGAAGGGCAA
GAGCTACCCCAAGATCAACCAGACCTACATCAACGACAAGGGCAAGGAGGTGCTGGT
GCTGTGGGGCATCCACCATCCTCCCACCATCGCCGACCAGCAGAGCCTGTACCAGAAC
GCCGACGCCTACGTGTTCGTGGGCACCAGCAGGTACAGCAAGAAGTTCAAGCCCGAG
ATCGCCACCAGGCCCAAGGTGAGGGACCAGGAGGGCAGGATGAACTACTACTGGACC
CTGGTGGAGCCCGGCGACAAGATCACCTTCGAGGCCACCGGCAACCTGGTGGCCCCT
AGGTACGCCTTCACAATGGAGAGGGACGCCGGCAGCGGCATCATCATTAGCGACACC
CCTGTGCACGACTGCAACACCACCTGCCAGACCCCTGAGGGCGCCATCAACACCAGCC
TGCCCTTCCAGAACGTGCACCCCATCACCATCGGCAAGTGCCCCAAGTACGTGAAGTC
CACCAAGCTGAGGCTGGCCACCGGCCTGAGGAACGTGCCCAGCATCCAGAGCAGGGG
CCTGTTCGGCGCCATCGCCGGCTTCATCGAGGGCGGCTGGACCGGCATGGTGGACGG
CTGGTACGGCTACCACCACCAGAACGAGCAGGGCAGCGGCTACGCCGCCGACCTGAA
GTCCACCCAGAACGCCATCGACAAGATCACCAACAAGGTGAACAGCGTGATCGAGAAG
ATGAACACCCAGTTCACCGCCGTGGGCAAGGAGTTCAACCACCTGGAGAAGAGGATC
GAGAACCTGAACAAGAAGGTGGACGACGGCTTCCTGGACATCTGGACCTACAACGCC
GAGCTGCTGGTGCTGCTGGAGAACGAGAGGACCCTGGACTACCACGACAGCAACGTG
AAGAACCTGTACGAGAAGGTGAGGAACCAGCTGAAGAACAACGCCAAGGAGATCGGC
AACGGCTGCTTCGAGTTCTACCACAAGTGCGACAACACCTGCATGGAGAGCGTGAAGA
ACGGCACCTACGACTACCCCAAGTACAGCGAGGAGGCCAAGCTGAACAGGGAGAAGA
TCGACGGCGTGAAGCTGGACAGCACCAGGATCTACCAGATCCTGGCCATCTACAGCAC
CGTGGCCAGCAGCCTGGTGCTGGTGGTGAGCCTGGGCGCCATCAGCTTCTGGATGTG
CAGCAACGGCAGCCTGCAGTGCAGGATCTGCATCTGATAGTAAACTCGAGTATGTTACG
TGCAAAGGTGATTGTCACCCCCCGAAAGACCATATTGTGACACACCCTCAGTATCACGCCCA
AACATTTACAGCCGCGGTGTCAAAAACCGCGTGGACGTGGTTAACATCCCTGCTGGGAGGA
TCAGCCGTAATTATTATAATTGGCTTGGTGCTGGCTACTATTGTGGCCATGTACGTGCTGACC
AACCAGAAACATAATTGAATACAGCAGCAATTGGCAAGCTGCTTACATAGAACTCGCGGCG
ATTGGCATGCCGCCTTAAAATTTTTATTTTATTTTTTCTTTTCTTTTCCGAATCGGATTTTGTT
TTTAATATTTC
SEQ ID NO: 4 (B Yamagata bicistronic construct; self-replicating RNA for
expression of NA/HA from B Yamagata influenza strain)
ATGGGCGGCGCATGAGAGAAGCCCAGACCAATTACCTACCCAAAATGGAGAAAGTTCACGTT
GACATCGAGGAAGACAGCCCATTCCTCAGAGCTTTGCAGCGGAGCTTCCCGCAGTTTGAGGTAGA
AGCCAAGCAGGTCACTGATAATGACCATGCTAATGCCAGAGCGTTTTCGCATCTGGCTTCAAAACT
GATCGAAACGGAGGTGGACCCATCCGACACGATCCTTGACATTGGAAGTGCGCCCGCCCGCAGA
ATGTATTCTAAGCACAAGTATCATTGTATCTGTCCGATGAGATGTGCGGAAGATCCGGACAGATTG
TATAAGTATGCAACTAAGCTGAAGAAAAACTGTAAGGAAATAACTGATAAGGAATTGGACAAGAAAA
TGAAGGAGCTGGCCGCCGTCATGAGCGACCCTGACCTGGAAACTGAGACTATGTGCCTCCACGA
CGACGAGTCGTGTCGCTACGAAGGGCAAGTCGCTGTTTACCAGGATGTATACGCCGTCGACGGC
CCCACCAGCCTGTACCACCAGGCCAACAAGGGCGTGAGGGTGGCCTACTGGATCGGCTTCGACA
CCACACCCTTCATGTTCAAGAACCTGGCCGGCGCCTACCCCAGCTACAGCACCAACTGGGCCGAC
GAGACAGTGCTGACCGCCAGGAACATCGGCCTGTGCAGCAGCGACGTGATGGAGAGGAGCCGG
AGGGGCATGAGCATCCTGAGGAAGAAGTACCTGAAGCCCAGCAACAACGTGCTGTTCAGCGTGG
GCAGCACCATCTACCACGAGAAGAGGGACCTGCTGAGGAGCTGGCACCTGCCCAGCGTGTTCCA
CCTGAGGGGCAAGCAGAACTACACCTGCAGGTGCGAGACAATCGTGAGCTGCGACGGCTACGTG
GTGAAGAGGATCGCCATCAGCCCCGGCCTGTACGGCAAGCCCAGCGGCTACGCCGCCACCATGC
ACAGGGAGGGCTTCCTGTGCTGCAAGGTGACCGACACCCTGAACGGCGAGAGGGTGAGCTTCCC
CGTGTGCACCTACGTGCCCGCCACCCTGTGCGACCAGATGACCGGCATCCTGGCCACCGACGTG
AGCGCCGACGACGCCCAGAAGCTGCTGGTGGGCCTGAACCAGAGGATCGTGGTGAACGGCAGG
ACCCAGAGGAACACCAACACCATGAAGAACTACCTGCTGCCCGTGGTGGCCCAGGCCTTCGCCA
GGTGGGCCAAGGAGTACAAGGAGGACCAGGAGGACGAGAGGCCCCTGGGCCTGAGGGACCGAC
AGCTGGTGATGGGCTGCTGCTGGGCCTTCAGGCGGCACAAGATCACCAGCATCTACAAGAGGCC
CGACACCCAGACCATCATCAAGGTGAACAGCGACTTCCACAGCTTCGTGCTGCCCAGGATCGGCA
GCAACACCCTGGAGATCGGCCTGAGGACCCGGATCAGGAAGATGCTGGAGGAGCACAAGGAGCC
CAGCCCTCTGATCACCGCCGAGGACGTGCAGGAGGCCAAGTGCGCCGCCGACGAGGCCAAGGA
GGTGAGGGAGGCCGAGGAGCTGAGGGCCGCCCTGCCTCCCCTGGCCGCCGACGTGGAGGAGC
CCACCCTGGAGGCCGACGTGGACCTGATGCTGCAGGAGGCCGGCGCCGGCAGCGTGGAGACAC
CCAGGGGCCTGATCAAGGTGACCAGCTACGACGGCGAGGACAAGATCGGCAGCTACGCCGTGCT
CAGCCCTCAGGCCGTGCTGAAGTCCGAGAAGCTGAGCTGCATCCACCCTCTGGCCGAGCAGGTG
ATCGTGATCACCCACAGCGGCAGGAAGGGCAGGTACGCCGTGGAGCCCTACCACGGCAAGGTG
GTGGTCCCCGAGGGCCACGCCATCCCCGTGCAGGACTTCCAGGCCCTGAGCGAGAGCGCCACC
ATCGTGTATAACGAGAGGGAGTTCGTGAACAGGTACCTGCACCACATCGCCACCCACGGCGGCG
CCCTGAACACCGACGAGGAGTACTACAAGACCGTGAAGCCCAGCGAGCACGACGGCGAGTACCT
GTACGACATCGACAGGAAGCAGTGCGTGAAGAAGGAGCTGGTGACCGGCCTGGGCCTGACCGG
CGAGCTGGTGGACCCTCCCTTCCACGAGTTCGCCTACGAGAGCCTGAGGACCAGGCCCGCCGCT
CCCTACCAGGTGCCCACCATCGGCGTGTACGGCGTGCCCGGCAGCGGCAAGAGCGGCATCATCA
AGAGCGCCGTGACCAAGAAGGACCTGGTGGTGAGCGCCAAGAAGGAGAACTGCGCCGAGATCAT
CAGGGACGTGAAGAAGATGAAGGGCCTGGACGTGAACGCCAGGACCGTGGACAGCGTGCTCCTG
AACGGCTGCAAGCACCCCGTGGAGACACTGTATATCGACGAGGCCTTCGCCTGCCACGCCGGCA
CCCTGAGGGCCCTGATCGCCATCATCAGGCCCAAGAAGGCCGTGCTGTGCGGCGACCCCAAGCA
GTGCGGCTTCTTCAACATGATGTGCCTGAAGGTGCACTTCAACCACGAGATCTGCACCCAGGTGTT
CCACAAGAGCATCAGCAGGCGGTGCACCAAGAGCGTGACCAGCGTGGTGAGCACCCTGTTCTAC
GACAAGAAGATGAGGACCACCAACCCCAAGGAGACAAAGATCGTGATCGACACCACCGGCAGCA
CCAAGCCCAAGCAGGACGACCTGATCCTGACCTGCTTCAGGGGCTGGGTGAAGCAGCTGCAGAT
CGACTACAAGGGCAACGAGATCATGACCGCCGCCGCTAGCCAGGGCCTGACCAGGAAGGGCGT
GTACGCCGTGAGGTACAAGGTGAACGAGAATCCCCTGTACGCCCCTACCAGCGAGCACGTGAAC
GTCCTGCTGACCAGGACCGAGGACAGGATCGTGTGGAAGACCCTGGCCGGCGACCCCTGGATCA
AGACCCTGACCGCCAAGTACCCCGGCAACTTCACCGCCACCATCGAGGAGTGGCAGGCCGAGCA
CGACGCCATCATGAGGCACATCCTGGAGAGGCCCGACCCCACCGACGTGTTCCAGAACAAGGCC
AACGTGTGCTGGGCCAAGGCCCTGGTGCCCGTGCTGAAGACCGCCGGCATCGACATGACCACCG
AGCAGTGGAACACCGTGGACTACTTCGAGACAGACAAGGCCCACAGCGCCGAGATCGTGCTGAA
CCAGCTGTGCGTGAGGTTCTTCGGCCTGGACCTGGACAGCGGCCTGTTCAGCGCCCCTACCGTG
CCCCTGAGCATCAGGAACAACCACTGGGACAACAGCCCCAGCCCCAACATGTACGGCCTGAACAA
GGAGGTGGTGAGGCAGCTGAGCAGGCGGTACCCTCAGCTGCCCAGGGCCGTGGCCACCGGCAG
GGTGTACGACATGAACACCGGCACCCTGAGGAACTACGACCCCAGGATCAACCTGGTGCCCGTG
AACAGGCGGCTGCCACACGCCCTGGTGCTGCACCACAACGAGCACCCTCAGAGCGACTTCAGCA
GCTTCGTGAGCAAGCTGAAGGGCAGGACCGTGCTGGTGGTGGGCGAGAAGCTGAGCGTGCCCG
GCAAGATGGTGGACTGGCTGAGCGACAGGCCCGAGGCCACCTTCCGGGCCAGGCTGGACCTGG
GCATCCCCGGCGACGTGCCCAAGTACGACATCATCTTCGTGAACGTGAGGACCCCTTACAAGTAC
CACCACTACCAGCAGTGCGAGGACCACGCCATCAAGCTGAGCATGCTGACCAAGAAGGCCTGCC
TGCACCTGAACCCCGGCGGCACCTGCGTGAGCATCGGCTACGGCTACGCCGACAGGGCCAGCG
AGAGCATCATCGGCGCCATCGCCAGGCTGTTCAAGTTCAGCAGGGTGTGCAAGCCCAAGAGCAG
CCTGGAGGAGACAGAGGTGCTGTTCGTGTTCATCGGCTACGACCGGAAGGCCAGGACCCACAAC
CCCTACAAGCTGAGCAGCACCCTGACCAACATCTACACCGGCAGCAGGCTGCACGAGGCCGGCT
GCGCCCCTAGCTACCACGTGGTGAGGGGCGACATCGCCACCGCCACCGAGGGCGTGATCATCAA
CGCCGCCAACAGCAAGGGCCAGCCCGGCGGCGGGGTGTGCGGCGCCCTGTATAAGAAGTTCCC
CGAGAGCTTCGACCTGCAGCCCATCGAGGTGGGCAAGGCCAGGCTGGTGAAGGGCGCCGCCAA
GCACATCATCCACGCCGTGGGCCCCAACTTCAACAAGGTGAGCGAGGTGGAGGGCGACAAGCAG
CTGGCCGAGGCCTACGAGAGCATCGCCAAGATCGTGAACGACAACAACTACAAGAGCGTGGCCA
TCCCTCTGCTGAGCACCGGCATCTTCAGCGGCAACAAGGACAGGCTGACCCAGAGCCTGAACCA
CCTGCTGACCGCCCTGGACACCACCGACGCCGACGTGGCCATCTACTGCAGGGACAAGAAGTGG
GAGATGACCCTGAAGGAGGCCGTGGCCAGGCGGGAGGCCGTGGAGGAGATCTGCATCAGCGAC
GACAGCAGCGTGACGGAGCCCGACGCCGAGCTGGTGAGGGTGCACCCCAAGAGCAGCCTGGCC
GGCAGGAAGGGCTACAGCACCAGCGACGGCAAGACCTTCAGCTACCTGGAGGGCACCAAGTTCC
ACCAGGCCGCCAAGGACATCGCCGAGATCAACGCCATGTGGCCCGTGGCCACCGAGGCCAACGA
GCAGGTGTGCATGTATATCCTGGGCGAGAGCATGAGCAGCATCAGGAGCAAGTGCCCCGTGGAG
GAGAGCGAGGCCAGCACCCCTCCCAGCACCCTGCCCTGCCTGTGCATCCACGCCATGACCCCTG
AGAGGGTGCAGCGGCTGAAGGCCAGCAGGCCCGAGCAGATCACCGTGTGCAGCAGCTTCCCTCT
GCCCAAGTACCGGATCACCGGCGTGCAGAAGATCCAGTGCAGCCAGCCCATCCTGTTCAGCCCC
AAGGTGCCCGCCTACATCCACCCCAGGAAGTACCTGGTGGAGACACCCCCCGTGGACGAGACAC
CCGAGCCCAGCGCCGAGAACCAGAGCACCGAGGGCACCCCTGAGCAGCCTCCCCTGATCACCG
AGGACGAGACAAGGACCAGGACGCCCGAGCCCATCATCATTGAGGAGGAAGAGGAGGACAGCAT
CAGCCTGCTGAGCGACGGCCCCACCCACCAGGTGCTGCAGGTGGAGGCCGACATCCACGGCCC
TCCCAGCGTGAGCAGCTCCAGCTGGAGCATCCCTCACGCCAGCGACTTCGACGTGGACAGCCTG
AGCATCCTGGACACCCTGGAGGGCGCCAGCGTGACCAGCGGCGCCACCAGCGCCGAGACAAAC
AGCTACTTCGCCAAGAGCATGGAGTTCCTGGCCAGGCCCGTGCCCGCCCCTAGGACCGTGTTCA
GGAACCCTCCCCACCCCGCCCCTAGGACCAGGACCCCTAGCCTGGCCCCTAGCAGGGCCTGCAG
CAGGACCAGCCTGGTGAGCACCCCTCCCGGCGTGAACCGGGTGATCACCAGGGAGGAGCTGGA
GGCCCTGACCCCTAGCAGGACCCCTAGCAGGAGCGTGAGCAGGACCAGCCTGGTGAGCAACCCT
CCCGGCGTGAACCGGGTGATCACCAGGGAGGAGTTCGAGGCCTTCGTGGCCCAGCAGCAAAGG
CGGTTCGACGCCGGCGCCTACATCTTCAGCAGCGACACCGGCCAGGGCCACCTGCAGCAGAAGT
CCGTGAGGCAGACCGTGCTGAGCGAGGTGGTCCTGGAGAGGACGGAGCTGGAGATCAGCTACG
CCCCTAGGCTGGACCAGGAGAAGGAGGAGCTGCTGAGGAAGAAGCTGCAGCTGAACCCCACCCC
TGCCAACAGGAGCAGGTACCAGAGCAGGAAGGTGGAGAACATGAAGGCCATCACCGCCAGGCGG
ATCCTGCAGGGCCTGGGCCACTACCTGAAGGCCGAGGGCAAGGTGGAGTGCTACAGGACCCTGC
ACCCCGTGCCCCTGTACTCCAGCTCCGTGAACAGGGCCTTCAGCAGCCCCAAGGTGGCCGTGGA
GGCCTGCAACGCCATGCTGAAGGAGAACTTCCCCACCGTGGCCAGCTACTGCATCATCCCCGAGT
ACGACGCCTACCTGGACATGGTGGACGGCGCCAGCTGCTGCCTGGACACCGCCAGCTTCTGCCC
CGCCAAGCTGAGGAGCTTCCCCAAGAAGCACAGCTACCTGGAGCCCACCATCAGGAGCGCCGTG
CCCAGCGCCATCCAGAACACCCTGCAGAACGTGCTGGCCGCCGCTACCAAGAGGAACTGCAACG
TGACCCAGATGAGGGAGCTGCCCGTGCTGGACAGCGCCGCCTTCAACGTGGAGTGCTTCAAGAA
GTACGCCTGCAACAACGAGTACTGGGAGACATTCAAGGAGAACCCCATCAGGCTGACCGAGGAG
AACGTGGTGAACTACATCACCAAGCTGAAGGGCCCCAAGGCCGCCGCTCTGTTCGCCAAGACCCA
CAACCTGAACATGCTCCAGGACATCCCTATGGACAGGTTCGTGATGGACCTGAAGAGGGACGTGA
AGGTGACCCCTGGCACCAAGCACACCGAGGAGAGGCCCAAGGTGCAGGTGATCCAGGCCGCCG
ACCCTCTGGCCACCGCCTACCTGTGCGGCATCCACAGGGAGCTGGTGAGGCGGCTGAACGCCGT
CCTGCTGCCCAACATCCACACCCTGTTCGACATGAGCGCCGAGGACTTCGACGCCATCATCGCCG
AGCACTTCCAGCCCGGCGACTGCGTGCTGGAGACAGACATCGCCAGCTTCGACAAGAGCGAGGA
CGACGCTATGGCCCTGACCGCCCTGATGATCCTGGAGGACCTGGGCGTGGACGCCGAGCTGCTG
ACCCTGATCGAGGCCGCCTTCGGCGAGATCAGCAGCATCCACCTGCCCACCAAGACCAAGTTCAA
GTTCGGCGCCATGATGAAGTCCGGCATGTTCCTGACCCTGTTCGTGAACACCGTGATCAACATCG
TGATCGCCAGCAGGGTGCTGCGGGAGAGGCTGACCGGCAGCCCCTGCGCCGCCTTCATCGGCG
ACGACAACATCGTGAAGGGCGTGAAGTCCGACAAGCTGATGGCCGACAGGTGCGCCACCTGGCT
GAACATGGAGGTGAAGATCATCGACGCCGTGGTGGGCGAGAAGGCCCCTTACTTCTGCGGCGGC
TTCATCCTGTGCGACAGCGTGACCGGCACCGCCTGCAGGGTGGCCGACCCTCTGAAGAGGCTGT
TCAAGCTGGGCAAGCCCCTGGCCGCCGACGACGAGCACGACGACGATAGGCGGAGGGCCCTGC
ACGAGGAGAGCACCAGGTGGAACCGGGTGGGCATCCTGAGCGAGCTGTGCAAGGCCGTGGAGA
GCAGGTACGAGACAGTGGGCACCAGCATCATCGTGATGGCCATGACCACCCTGGCCAGCAGCGT
CAAGTCCTTCAGCTACCTGAGGGGGGCCCCTATAACTCTCTACGGCTAACCTGAATGGACTACG
ACATAGTCTAGTCCGCCAAGGCCGCCACCATGCTGCCTAGCACCATCCAGACCCTGACAC
TGTTCCTGACCAGCGGCGGCGTGCTGCTGAGCCTGTACGTGAGCGCCAGCCTGAGCT
ACCTGCTGTACTCCGACATCCTGCTGAAGTTCAGCAGGACCGAGGTGACCGCCCCTAT
CATGCCCCTGGACTGCGCCAACGCCAGCAACGTGCAGGCCGTGAACAGGAGCGCCAC
CAAGGGCGTGACCCCTCTGCTGCCCGAGCCCGAGTGGACCTACCCCAGGCTGAGCTG
CCCCGGCAGCACCTTCCAGAAGGCCCTGCTGATCAGCCCTCACAGGTTCGGCGAGACA
AAGGGCAACAGCGCCCCTCTGATCATCAGGGAGCCCTTCATCGCCTGCGGCCCCAAGG
AGTGCAAGCACTTCGCCCTGACCCACTACGCCGCCCAGCCCGGCGGCTACTACAACGG
CACCAGGGAGGACAGGAACAAGCTGAGGCACCTGATCAGCGTGAAGCTGGGCAAGAT
CCCCACCGTGGAGAACAGCATCTTCCACATGGCCGCCTGGAGCGGCAGCGCCTGCCA
CGACGGCAGGGAGTGGACCTACATCGGCGTGGACGGCCCCGACAGCAACGCCCTGCT
GAAGATCAAGTACGGCGAGGCCTACACCGACACCTACCACAGCTACGCCAAGAACATC
CTGAGGACCCAGGAGAGCGCCTGCAACTGCATCGGCGGCGACTGCTACCTGATGATC
ACCGACGGCCCCGCCAGCGGCATCAGCGAGTGCAGGTTCCTGAAGATCAGGGAGGGC
AGGATCATCAAGGAGATCTTCCCCACCGGCAGGGTGAAGCACACCGAGGAGTGCACC
TGCGGCTTCGCCAGCAACAAGACCATCGAGTGCGCCTGCAGGGACAACAGCTACACC
GCCAAGAGGCCCTTCGTGAAGCTGAACGTGGAGACAGACACCGCCGAGATCAGGCTG
ATGTGCACCAAGACCTACCTGGACACCCCTAGGCCCAACGACGGCAGCATCACCGGCC
CCTGCGAGAGCGACGGCGACGAGGGCAGCGGCGGCATCAAGGGCGGCTTCGTGCACC
AGAGGATGGCCAGCAAGATCGGCAGGTGGTACAGCAGGACCATGAGCAAGACCAAGA
GGATGGGCATGGGCCTGTACGTGAAGTACGACGGCGACCCCTGGACCGACAGCGAGG
CCCTGGCCCTGAGCGGCGTGATGGTGAGCATGGAGGAGCCCGGCTGGTACAGCTTCG
GCTTCGAGATCAAGGACAAGAAGTGCGACGTGCCCTGCATCGGCATCGAGATGGTGC
ACGACGGCGGCAAGACCACCTGGCACAGCGCCGCCACCGCCATCTACTGCCTGATGG
GCAGCGGCCAGCTGCTGTGGGACACCGTGACCGGCGTGAACATGACCCTGTGATAGT
AAACTCGAAGCAGTGTTAAATCATTCAGCTACCTGAGAGGGGCCCCTATAACTCTCTACGGC
TAACCTGAATGGACTACGACATAGTCTAGTCCGCCAAGGCCGCCACCATGAAGGCCATCAT
CGTGCTGCTGATGGTGGTGACCTCTAACGCCGACAGGATCTGCACCGGCATCACCAGC
AGCAACAGCCCTCACGTGGTGAAGACCGCCACCCAGGGCGAGGTGAACGTGACCGGC
GTGATCCCTCTGACCACCACACCCACCAAGAGCTACTTCGCCAACCTGAAGGGCACCA
GGACCAGGGGCAAGCTGTGCCCCGACTGCCTGAACTGCACCGACCTGGACGTGGCCC
TGGGCAGGCCCATGTGCGTGGGCACCACACCCAGCGCCAAGGCCAGCATCCTGCACG
AGGTGAGGCCCGTGACCAGCGGCTGCTTCCCCATCATGCACGACAGGACCAAGATCA
GGCAGCTGCCCAACCTGCTGAGGGGCTACGAGAAGATCAGGCTGAGCACCCAGAACG
TGATCGACGCCGAGAAGGCCCCTGGCGGCCCCTACAGGCTGGGCACCAGCGGCAGCT
GCCCCAACGCCACCAGCAAGATCGGCTTCTTCGCCACAATGGCCTGGGCCGTGCCCAA
GGACAACTACAAGAACGCCACCAATCCCCTGACCGTGGAGGTGCCCTACATCTGCACC
GAGGGCGAGGACCAGATCACCGTGTGGGGCTTCCACAGCGACAACAAGACCCAGATG
AAGTCCCTGTACGGCGACAGCAATCCCCAGAAGTTCACCAGCAGCGCCAACGGCGTGA
CCACCCACTACGTGAGCCAGATCGGCGACTTCCCCGACCAGACCGAGGACGGCGGCC
TGCCCCAGAGCGGCAGGATCGTGGTGGACTACATGATGCAGAAGCCCGGCAAGACCG
GCACCATCGTGTACCAGAGGGGCGTGCTGCTGCCCCAGAAGGTGTGGTGCGCCAGCG
GCAGGAGCAAGGTGATCAAGGGCAGCCTGCCCCTGATCGGCGAGGCCGACTGCCTGC
ACGAGGAGTACGGCGGCCTGAACAAGAGCAAGCCCTACTACACCGGCAAGCACGCCA
AGGCCATCGGCAACTGCCCCATCTGGGTGAAGACCCCTCTGAAGCTGGCCAACGGCAC
CAAGTACAGGCCTCCCGCCAAGCTGCTGAAGGAGAGGGGCTTCTTCGGCGCCATCGC
CGGCTTCCTGGAGGGCGGCTGGGAGGGCATGATCGCCGGCTGGCACGGCTACACCAG
CCACGGCGCCCACGGCGTGGCCGTGGCCGCCGACCTGAAGTCCACCCAGGAGGCCAT
CAACAAGATCACCAAGAACCTGAACAGCCTGAGCGAGCTGGAGGTGAAGAACCTGCA
GAGGCTGAGCGGCGCTATGGACGAGCTGCACAACGAGATCCTGGAGCTGGACGAGAA
GGTGGACGACCTGAGGGCCGACACCATCAGCAGCCAGATCGAGCTGGCCGTGCTGCT
GAGCAACGAGGGCATCATCAACAGCGAGGACGAGCACCTGCTGGCCCTGGAGAGGAA
GCTGAAGAAGATGCTGGGCCCCAGCGCCGTGGACATCGGCAACGGCTGCTTCGAGAC
AAAGCACAAGTGCAACCAGACCTGCCTGGACAGGATCGCCGCCGGCACCTTCAACGCC
GGCGAGTTCAGCCTGCCCACCTTCGACAGCCTGAACATCACCGCCGCCAGCCTGAACG
ACGACGGCCTGGACAACCACACCATCCTGCTGTACTACAGCACCGCCGCCAGCAGCCT
GGCCGTGACCCTGATGCTGGCCATCTTCATCGTGTATATGGTGAGCAGGGACAACGTG
AGCTGCAGCATCTGCCTGTGATAGTAAACTCGAGTATGTTACGTGCAAAGGTGATTGTCA
CCCCCCGAAAGACCATATTGTGACACACCCTCAGTATCACGCCCAAACATTTACAGCCGCG
GTGTCAAAAACCGCGTGGACGTGGTTAACATCCCTGCTGGGAGGATCAGCCGTAATTATTAT
AATTGGCTTGGTGCTGGCTACTATTGTGGCCATGTACGTGCTGACCAACCAGAAACATAATT
GAATACAGCAGCAATTGGCAAGCTGCTTACATAGAACTCGCGGCGATTGGCATGCCGCCTT
AAAATTTTTATTTTATTTTTTCTTTTCTTTTCCGAATCGGATTTTGTTTTTAATATTTC
SEQ ID NO: 5 (Codon optimized NA from H1N1 influenza strain)
ATGCTGCCTAGCACCATCCAGACCCTGACACTGTTCCTGACCAGCGGCGGCGTGCTGCTGAG
CCTGTACGTGAGCGCCAGCCTGAGCTACCTGCTGTACTCCGACATCCTGCTGAAGTTCAGCC
AGACCGAGATCACCGCCCCTACCATGCCCCTGGACTGCGCCAACGCCAGCAACGTGCAGGC
CGTGAACAGGAGCGCCACCAAGGGCGCCACCCTGCTGCTCCCCGAGCCCGAGTGGACCTAC
CCCAGGCTGAGCTGCCCCGGCAGCACCTTCCAGAAGGCCCTGCTGATCAGCCCTCACAGGTT
CGGCGAGACAAAGGGCAACAGCGCCCCTCTGATCATCAGGGAGCCCTTCGTGGCCTGCGGC
CCCAACGAGTGCAAGCACTTCGCCCTGACCCACTACGCCGCCCAGCCCGGCGGCTACTACA
ACGGCACCAGGGGCGACAGGAACAAGCTGAGGCACCTGATCAGCGTGAAGCTGGGCAAGA
TCCCCACCGTGGAGAACAGCATCTTCCACATGGCCGCCTGGAGCGGCAGCGCCTGCCACGA
CGGCAAGGAGTGGACCTACATCGGCGTGGACGGCCCCGACAACAACGCCCTGCTGAAGGTG
AAGTACGGCGAGGCCTACACCGACACCTACCACAGCTACGCCAACAACATCCTGAGGACCC
AGGAGAGCGCCTGCAACTGCATCGGCGGCAACTGCTACCTGATGATCACCGACGGCAGCGC
CAGCGGCGTGAGCGAGTGCAGGTTCCTGAAGATCAGGGAGGGCAGGATCATCAAGGAGAT
CTTCCCCACCGGCAGGGTGAAGCACACCGAGGAGTGCACCTGCGGCTTCGCCAGCAACAAG
ACCATCGAGTGCGCCTGCAGGGACAACAGGTACACCGCCAAGAGGCCCTTCGTGAAGCTGA
ACGTGGAGACAGACACCGCCGAGATCAGGCTGATGTGCACCGACACCTACCTGGACACCCC
TAGGCCCAACGACGGCAGCATCACCGGCCCCTGCGAGAGCGACGGCGACGAGGGCAGCGG
CGGCATCAAGGGCGGCTTCGTGCACCAGAGGATGAAGTCCAAGATCGGCAGGTGGTACAGC
AGGACCATGAGCAAGACCGAGAGGATGGGCATGGGCCTGTACGTGAAGTACGGCGGCGAC
CCCTGGGCCGACAGCGACGCCCTGGTGTTCAGCGGCGTGATGATCAGCATGAAGGAGCCCG
GCTGGTACAGCTTCGGCTTCGAGATCAAGGACAAGAAGTGCGACGTGCCCTGCATCGGCAT
CGAGATGGTGCACGACGGCGGCAAGGAGACATGGCACAGCGCCGCCACCGCCATCTACTGC
CTGATGGGCAGCGGCCAGCTGCTGTGGGACACCATCACCGGCGTGGACATGGCCCTGTGAT
AGTAA
SEQ ID NO: 6 (Codon optimized NA from H3N2 influenza strain)
ATGAATCCTAACCAGAAGATCATCACCATCGGCAGCGTGAGCCTGACCATCAGCACCATCT
GCTTCTTCATGCAGATCGCCATCCTGATCACCACCGTGACCCTGCACTTCAAGCAGTACGAG
TTCAACAGCCCTCCCAACAACCAGGTGATGCTGTGCGAGCCCACCATCATCGAGAGGAACA
TCACCGAGATCGTGTACCTGACCAACACCACCATCGAGAAGGAGATCTGCCCCAAGCCCGC
CGAGTACAGGAACTGGAGCAAGCCCCAGTGCGGCATCACCGGCTTCGCCCCTTTCAGCAAG
GACAACAGCATCAGGCTGAGCGCCGGCGGCGACATCTGGGTGACCAGGGAGCCCTACGTGA
GCTGCGACCTGGACAAGTGCTACCAGTTCGCCCTGGGCCAGGGCACCACCCTGAACAACGT
GCACAGCAACAACACCGTGAGGGACAGGACCCCTTACCGGACCCTGCTGATGAACGAGCTG
GGCGTGCCCTTCCACCTGGGCACCAAGCAGGTGTGCATCGCCTGGAGCAGCTCCAGCTGCC
ACGACGGCAAGGCCTGGCTGCACGTGTGCATCACCGGCGACGACAAGAACGCCACCGCCAG
CTTCATCTACAACGGCAGGCTGGTGGACAGCGTGGTGAGCTGGAGCAACGACATCCTGAGG
ACCCAGGAGAGCGAGTGCGTGTGCATCAACGGCACCTGCACCGTGGTGATGACCGACGGCA
ACGCCACCGGCAAGGCCGACACCAAGATCCTGTTCATCGAGGAGGGCAAGATCGTGCACAC
CAGCAAGCTGAGCGGCAGCGCCCAGCACGTGGAGGAGTGCAGCTGCTACCCCAGGTACCCC
GGCGTGAGGTGCGTGTGCAGGGACAACTGGAAGGGCAGCAACCGGCCCATCATCGACATCA
ACATCAAGGACCACAGCATCGTCAGCAGGTACGTGTGCAGCGGCCTGGTGGGCGACACCCC
TAGAAAGAGCGACAGCAGCTCCAGCAGCCACTGCCTGAACCCCAACAACGAGAAGGGCGA
CCACGGCGTGAAGGGCTGGGCCTTCGACGACGGCAACGACGTGTGGATGGGCAGGACCATC
AACGAGACAAGCAGGCTGGGCTACGAGACATTCAAGGTGGTGGAGGGCTGGAGCAACCCC
AAGAGCAAGCTGCAGATCAACAGGCAGGTGATCGTGGACAGGGGCGACAGGAGCGGCTAC
AGCGGCATCTTCAGCGTGGAGGGCAAGAGCTGCATCAACCGGTGCTTCTACGTGGAGCTGA
TCAGGGGCAGGAAGGAGGAGACAGAGGTGCTGTGGACCAGCAACAGCATCGTGGTGTTCTG
CGGCACCAGCGGCACCTACGGCACCGGCAGCTGGCCCGACGGCGCCAACCTGAGCCTGATG
CACATCTGATAGTAA
SEQ ID NO: 7 (Codon optimized NA from B Victoria influenza strain)
ATGAACCCCAACCAGAAGATCATCACCATCGGCAGCATCTGCATGACCATCGGCACCGCCA
ACCTGATCCTGCAGATCGGCAACATCATCAGCATCTGGGTGAGCCACAGCATCCAGATCGG
CAACCAGAGCCAGATCGAGACATGCAACAAGAGCGTGATCACCTACGAGAACAACACCTG
GGTGAACCAGACCTTCGTGAACATCAGCAACACCAACAGCGCCGCCAGGCAGAGCGTGGCC
AGCGTGAAGCTGGCCGGCAACAGCAGCCTGTGCCCCGTGAGCGGCTGGGCCATCTACAGCA
AGGACAACAGCGTGAGGATCGGCAGCAAGGGCGACGTGTTCGTGATCAGGGAGCCCTTCAT
CAGCTGCAGCCCTCTGGAGTGCAGGACCTTCTTCCTGACCCAGGGCGCCCTGCTGAACGACA
AGCACAGCAACGGCACCATCAAGGACAGGAGCCCCTACAGGACCCTGATGAGCTGCCCCAT
CGGCGAGGTGCCCAGCCCCTACAACAGCAGGTTCGAGAGCGTGGCCTGGAGCGCCAGCGCC
TGCCACGACGGCACCAACTGGCTGACCATCGGCATCAGCGGCCCCGACAGCGGCGCCGTGG
CCGTGCTGAAGTACAACGGCATCATCACCGACACCATCAAGAGCTGGAGGAACAAGATCCT
GAGGACCCAGGAGAGCGAGTGCGCCTGCGTGAACGGCAGCTGCTTCACCATCATGACCGAC
GGCCCCAGCGACGGCCAGGCCAGCTACAAGATCTTCAGGATCGAGAAGGGCAAGATCATCA
AGAGCGTGGAGATGAAGGCCCCTAACTACCACTACGAGGAGTGCAGCTGCTACCCCGACAG
CAGCGAGATCACCTGCGTGTGCAGGGACAACTGGCACGGCAGCAACAGGCCCTGGGTGAGC
TTCAACCAGAACCTGGAGTACCAGATGGGCTACATCTGCAGCGGCGTGTTCGGCGACAACC
CCAGGCCCAACGACAAGACCGGCAGCTGCGGCCCCGTGAGCAGCAACGGCGCCAACGGCG
TGAAGGGCTTCAGCTTCAAGTACGGCAACGGCGTGTGGATCGGCAGGACCAAGAGCATCAG
CAGCAGGAAGGGCTTCGAGATGATCTGGGACCCCAACGGCTGGACCGGCACCGACAACAA
GTTCAGCAAGAAGCAGGACATCGTGGGCATCAACGAGTGGAGCGGCTACAGCGGCAGCTTC
GTGCAGCACCCCGAGCTGACCGGCCTGAACTGCATCAGGCCCTGCTTCTGGGTGGAGCTGAT
CAGGGGCAGGCCCGAGGAGAACACCATCTGGACCAGCGGCAGCAGCATCAGCTTCTGCGGC
GTGGACAGCGACATCGTGGGCTGGAGCTGGCCCGACGGCGCCGAGCTGCCCTTCACCATCG
ACAAGTGATAGTAA
SEQ ID NO: 8 (Codon optimized NA from B Yamagata influenza strain)
ATGCTGCCTAGCACCATCCAGACCCTGACACTGTTCCTGACCAGCGGCGGCGTGCTGCTGAG
CCTGTACGTGAGCGCCAGCCTGAGCTACCTGCTGTACTCCGACATCCTGCTGAAGTTCAGCA
GGACCGAGGTGACCGCCCCTATCATGCCCCTGGACTGCGCCAACGCCAGCAACGTGCAGGC
CGTGAACAGGAGCGCCACCAAGGGCGTGACCCCTCTGCTGCCCGAGCCCGAGTGGACCTAC
CCCAGGCTGAGCTGCCCCGGCAGCACCTTCCAGAAGGCCCTGCTGATCAGCCCTCACAGGTT
CGGCGAGACAAAGGGCAACAGCGCCCCTCTGATCATCAGGGAGCCCTTCATCGCCTGCGGC
CCCAAGGAGTGCAAGCACTTCGCCCTGACCCACTACGCCGCCCAGCCCGGCGGCTACTACA
ACGGCACCAGGGAGGACAGGAACAAGCTGAGGCACCTGATCAGCGTGAAGCTGGGCAAGA
TCCCCACCGTGGAGAACAGCATCTTCCACATGGCCGCCTGGAGCGGCAGCGCCTGCCACGA
CGGCAGGGAGTGGACCTACATCGGCGTGGACGGCCCCGACAGCAACGCCCTGCTGAAGATC
AAGTACGGCGAGGCCTACACCGACACCTACCACAGCTACGCCAAGAACATCCTGAGGACCC
AGGAGAGCGCCTGCAACTGCATCGGCGGCGACTGCTACCTGATGATCACCGACGGCCCCGC
CAGCGGCATCAGCGAGTGCAGGTTCCTGAAGATCAGGGAGGGCAGGATCATCAAGGAGATC
TTCCCCACCGGCAGGGTGAAGCACACCGAGGAGTGCACCTGCGGCTTCGCCAGCAACAAGA
CCATCGAGTGCGCCTGCAGGGACAACAGCTACACCGCCAAGAGGCCCTTCGTGAAGCTGAA
CGTGGAGACAGACACCGCCGAGATCAGGCTGATGTGCACCAAGACCTACCTGGACACCCCT
AGGCCCAACGACGGCAGCATCACCGGCCCCTGCGAGAGCGACGGCGACGAGGGCAGCGGC
GGCATCAAGGGCGGCTTCGTGCACCAGAGGATGGCCAGCAAGATCGGCAGGTGGTACAGCA
GGACCATGAGCAAGACCAAGAGGATGGGCATGGGCCTGTACGTGAAGTACGACGGCGACC
CCTGGACCGACAGCGAGGCCCTGGCCCTGAGCGGCGTGATGGTGAGCATGGAGGAGCCCGG
CTGGTACAGCTTCGGCTTCGAGATCAAGGACAAGAAGTGCGACGTGCCCTGCATCGGCATC
GAGATGGTGCACGACGGCGGCAAGACCACCTGGCACAGCGCCGCCACCGCCATCTACTGCC
TGATGGGCAGCGGCCAGCTGCTGTGGGACACCGTGACCGGCGTGAACATGACCCTGTGATA
GTAA
SEQ ID NO: 9 (Codon optimized HA from H1N1 influenza strain)
ATGAAGGCCATCATCGTGCTGCTGATGGTGGTGACCTCTAACGCCGACAGGATCTGCACCG
GCATCACCAGCAGCAACAGCCCTCACGTGGTGAAGACCGCCACCCAGGGCGAGGTGAACGT
GACCGGCGTGATCCCTCTGACCACCACACCCACCAAGAGCCACTTCGCCAACCTGAAGGGC
ACCGAGACAAGGGGCAAGCTGTGCCCCAAGTGCCTGAACTGCACCGACCTGGACGTGGCCC
TGGGCAGGCCCAAGTGCACCGGCAAGATCCCCAGCGCCAGGGTGAGCATCCTGCACGAGGT
GAGGCCCGTGACCAGCGGCTGCTTCCCCATCATGCACGACAGGACCAAGATCAGGCAGCTG
CCCAACCTGCTGAGGGGCTACGAGCACGTGAGGCTGAGCACCCACAACGTGATCAACACCG
AGGACGCCCCTGGCGGCCCCTACGAGATCGGCACCAGCGGCAGCTGCCTGAACATCACCAA
CGGCAAGGGCTTCTTCGCCACAATGGCCTGGGCCGTGCCCAAGAACAAGACCGCCACCAAT
CCCCTGACCATCGAGGTGCCCTACATCTGCACCGAGGAGGAGGACCAGATCACCGTGTGGG
GCTTCCACAGCGACGACGAGACACAGATGGCCAGGCTGTACGGCGACAGCAAGCCCCAGA
AGTTCACCAGCAGCGCCAACGGCGTGACCACCCACTACGTGAGCCAGATCGGCGGCTTCCC
CAACCAGACCGAGGACGGCGGCCTGCCCCAGAGCGGCAGGATCGTGGTGGACTACATGGTG
CAGAAGTCCGGCAAGACCGGCACCATCACCTACCAGAGGGGCATCCTGCTGCCCCAGAAGG
TGTGGTGCGCCAGCGGCAAGAGCAAGGTGATCAAGGGCAGCCTGCCCCTGATCGGCGAGGC
CGACTGCCTGCACGAGAAGTACGGCGGCCTGAACAAGAGCAAGCCCTACTACACCGGCGAG
CACGCCAAGGCCATCGGCAACTGCCCCATCTGGGTGAAGACCCCTCTGAAGCTGGCCAACG
GCACCAAGTACAGGCCTCCCGCCAAGCTGCTGAAGGAGAGGGGCTTCTTCGGCGCCATCGC
CGGCTTCCTGGAGGGCGGCTGGGAGGGCATGATCGCCGGCTGGCACGGCTACACCAGCCAC
GGCGCCCACGGCGTGGCCGTGGCCGCCGACCTGAAGTCCACCCAGGAGGCCATCAACAAGA
TCACCAAGAACCTGAACAGCCTGAGCGAGCTGGAGGTGAAGAACCTGCAGAGGCTGAGCG
GCGCTATGGACGAGCTGCACAACGAGATCCTGGAGCTGGACGAGAAGGTGGACGACCTGA
GGGCCGACACCATCAGCAGCCAGATCGAGCTGGCCGTGCTGCTGAGCAACGAGGGCATCAT
CAACAGCGAGGACGAGCACCTGCTGGCCCTGGAGAGGAAGCTGAAGAAGATGCTGGGCCC
CAGCGCCGTGGAGATCGGCAACGGCTGCTTCGAGACAAAGCACAAGTGCAACCAGACCTGC
CTGGACAGGATCGCCGCCGGCACCTTCGACGCCGGCGAGTTCAGCCTGCCCACCTTCGACA
GCCTGAACATCACCGCCGCCAGCCTGAACGACGACGGCCTGGACAACCACACCATCCTGCT
GTACTACAGCACCGCCGCCAGCAGCCTGGCCGTGACCCTGATGATCGCCATCTTCGTGGTGT
ATATGGTGAGCAGGGACAACGTGAGCTGCAGCATCTGCCTGTGATAGTAA
SEQ ID NO: 10 (Codon optimized HA from H3N2 influenza strain)
ATGAAGACCATCATCGCCCTGAGCAACATCCTGTGCCTGGTGTTCGCCCAGAAGATCCCCGG
CAATGACAACAGCACCGCCACCCTGTGCCTGGGCCACCACGCCGTGCCCAACGGCACCATC
GTGAAGACCATCACCAACGACAGGATCGAGGTGACCAACGCCACCGAGCTGGTGCAGAAC
AGCAGCATCGGCGAGATCTGCGGCAGCCCTCACCAGATCCTGGACGGCGGCAACTGCACCC
TGATCGACGCCCTGCTGGGCGACCCTCAGTGCGACGGCTTCCAGAACAAGGAGTGGGACCT
GTTCGTGGAGAGGAGCAGGGCCAACAGCAACTGCTACCCCTACGACGTGCCCGACTACGCC
AGCCTGAGGAGCCTGGTGGCCAGCAGCGGCACCCTGGAGTTCAAGAACGAGAGCTTCAACT
GGACCGGCGTGAAGCAGAACGGCACCAGCAGCGCCTGCATCAGGGGCAGCAGCTCCAGCTT
CTTCAGCCGGCTGAATTGGCTCACCAGCCTGAACAACATCTACCCCGCCCAGAACGTGACC
ATGCCCAACAAGGAGCAGTTCGACAAGCTGTATATCTGGGGCGTGCACCACCCCGACACCG
ACAAGAACCAGATCAGCCTGTTCGCCCAGAGCAGCGGCAGGATCACCGTGAGCACCAAGAG
GAGCCAGCAGGCCGTGATCCCCAACATCGGCAGCAGGCCCAGGATCAGGGGCATCCCCAGC
AGGATCAGCATCTACTGGACCATCGTGAAGCCCGGCGACATCCTGCTGATCAACAGCACCG
GCAACCTGATCGCCCCTAGGGGCTACTTCAAGATCAGGAGCGGCAAGAGCAGCATCATGAG
GAGCGACGCCCCTATCGGCAAGTGCAAGAGCGAGTGCATCACCCCTAACGGCAGCATCCCC
AACGACAAGCCCTTCCAGAACGTGAACAGGATCACCTACGGCGCCTGCCCCAGGTACGTGA
AGCAGAGCACCCTGAAGCTGGCCACCGGCATGAGGAACGTGCCCGAGAAGCAGACCAGGG
GCATCTTCGGCGCCATCGCCGGCTTCATCGAGAACGGCTGGGAGGGCATGGTGGACGGCTG
GTACGGCTTCAGGCACCAGAACAGCGAGGGCAGGGGCCAGGCCGCCGACCTGAAGTCCAC
CCAGGCCGCCATCGACCAGATCAACGGCAAGCTGAACAGGCTGATCGGCAAGACCAACGA
GAAGTTCCACCAGATCGAGAAGGAGTTCAGCGAGGTGGAGGGCAGGGTGCAGGACCTGGA
GAAGTACGTGGAGGACACCAAGATCGACCTGTGGAGCTACAACGCCGAGCTGCTGGTGGCC
CTGGAGAACCAGCACACCATCGACCTGACCGACAGCGAGATGAACAAGCTGTTCGAGAAGA
CCAAGAAGCAGCTGAGGGAGAACGCCGAGGACATGGGCAACGGCTGCTTCAAGATCTACC
ACAAGTGCGACAACGCCTGCATCGGCAGCATCAGGAACGAGACATATGACCACAACGTGTA
CCGGGACGAGGCCCTGAACAACCGGTTCCAGATCAAGGGCGTGGAGCTGAAGTCCGGCTAC
AAGGACTGGATCCTGTGGATCAGCTTCGCCATGAGCTGCTTCCTGCTGTGCATCGCCCTGCT
GGGCTTCATCATGTGGGCCTGCCAGAAGGGCAACATCAGGTGCAACATCTGCATCTGATAG
TAA
SEQ ID NO: 11 (Codon optimized HA from B Victoria influenza strain)
ATGAAGGCCATCCTGGTGGTGATGCTGTATACCTTCACCACCGCTAACGCCGACACCCTGTG
CATCGGCTACCACGCCAACAACAGCACCGACACCGTGGACACCGTGCTGGAGAAGAACGTG
ACCGTGACCCACAGCGTGAACCTGCTGGAGGACAAGCACAACGGCAAGCTGTGCAAGCTGA
GGGGCGTGGCCCCTCTGCACCTGGGCAAGTGCAACATCGCCGGCTGGATCCTGGGCAACCC
CGAGTGCGAGAGCCTGAGCACCGCCAGGAGCTGGAGCTACATCGTGGAGACAAGCAACAG
CGACAACGGCACCTGCTACCCCGGCGACTTCATCAACTACGAGGAGCTGAGGGAGCAGCTG
AGCAGCGTGAGCAGCTTCGAGAGGTTCGAGATCTTCCCCAAGACCAGCAGCTGGCCCAACC
ACGACAGCGACAACGGCGTGACCGCCGCCTGCCCTCACGCCGGCGCCAAGAGCTTCTACAA
GAACCTGATCTGGCTGGTGAAGAAGGGCAAGAGCTACCCCAAGATCAACCAGACCTACATC
AACGACAAGGGCAAGGAGGTGCTGGTGCTGTGGGGCATCCACCATCCTCCCACCATCGCCG
ACCAGCAGAGCCTGTACCAGAACGCCGACGCCTACGTGTTCGTGGGCACCAGCAGGTACAG
CAAGAAGTTCAAGCCCGAGATCGCCACCAGGCCCAAGGTGAGGGACCAGGAGGGCAGGAT
GAACTACTACTGGACCCTGGTGGAGCCCGGCGACAAGATCACCTTCGAGGCCACCGGCAAC
CTGGTGGCCCCTAGGTACGCCTTCACAATGGAGAGGGACGCCGGCAGCGGCATCATCATTA
GCGACACCCCTGTGCACGACTGCAACACCACCTGCCAGACCCCTGAGGGCGCCATCAACAC
CAGCCTGCCCTTCCAGAACGTGCACCCCATCACCATCGGCAAGTGCCCCAAGTACGTGAAG
TCCACCAAGCTGAGGCTGGCCACCGGCCTGAGGAACGTGCCCAGCATCCAGAGCAGGGGCC
TGTTCGGCGCCATCGCCGGCTTCATCGAGGGCGGCTGGACCGGCATGGTGGACGGCTGGTA
CGGCTACCACCACCAGAACGAGCAGGGCAGCGGCTACGCCGCCGACCTGAAGTCCACCCAG
AACGCCATCGACAAGATCACCAACAAGGTGAACAGCGTGATCGAGAAGATGAACACCCAG
TTCACCGCCGTGGGCAAGGAGTTCAACCACCTGGAGAAGAGGATCGAGAACCTGAACAAGA
AGGTGGACGACGGCTTCCTGGACATCTGGACCTACAACGCCGAGCTGCTGGTGCTGCTGGA
GAACGAGAGGACCCTGGACTACCACGACAGCAACGTGAAGAACCTGTACGAGAAGGTGAG
GAACCAGCTGAAGAACAACGCCAAGGAGATCGGCAACGGCTGCTTCGAGTTCTACCACAAG
TGCGACAACACCTGCATGGAGAGCGTGAAGAACGGCACCTACGACTACCCCAAGTACAGCG
AGGAGGCCAAGCTGAACAGGGAGAAGATCGACGGCGTGAAGCTGGACAGCACCAGGATCT
ACCAGATCCTGGCCATCTACAGCACCGTGGCCAGCAGCCTGGTGCTGGTGGTGAGCCTGGG
CGCCATCAGCTTCTGGATGTGCAGCAACGGCAGCCTGCAGTGCAGGATCTGCATCTGATAGT
AA
SEQ ID NO: 12 (Codon optimized HA from B Yamagata influenza strain)
ATGAAGGCCATCATCGTGCTGCTGATGGTGGTGACCTCTAACGCCGACAGGATCTGCACCG
GCATCACCAGCAGCAACAGCCCTCACGTGGTGAAGACCGCCACCCAGGGCGAGGTGAACGT
GACCGGCGTGATCCCTCTGACCACCACACCCACCAAGAGCTACTTCGCCAACCTGAAGGGC
ACCAGGACCAGGGGCAAGCTGTGCCCCGACTGCCTGAACTGCACCGACCTGGACGTGGCCC
TGGGCAGGCCCATGTGCGTGGGCACCACACCCAGCGCCAAGGCCAGCATCCTGCACGAGGT
GAGGCCCGTGACCAGCGGCTGCTTCCCCATCATGCACGACAGGACCAAGATCAGGCAGCTG
CCCAACCTGCTGAGGGGCTACGAGAAGATCAGGCTGAGCACCCAGAACGTGATCGACGCCG
AGAAGGCCCCTGGCGGCCCCTACAGGCTGGGCACCAGCGGCAGCTGCCCCAACGCCACCAG
CAAGATCGGCTTCTTCGCCACAATGGCCTGGGCCGTGCCCAAGGACAACTACAAGAACGCC
ACCAATCCCCTGACCGTGGAGGTGCCCTACATCTGCACCGAGGGCGAGGACCAGATCACCG
TGTGGGGCTTCCACAGCGACAACAAGACCCAGATGAAGTCCCTGTACGGCGACAGCAATCC
CCAGAAGTTCACCAGCAGCGCCAACGGCGTGACCACCCACTACGTGAGCCAGATCGGCGAC
TTCCCCGACCAGACCGAGGACGGCGGCCTGCCCCAGAGCGGCAGGATCGTGGTGGACTACA
TGATGCAGAAGCCCGGCAAGACCGGCACCATCGTGTACCAGAGGGGCGTGCTGCTGCCCCA
GAAGGTGTGGTGCGCCAGCGGCAGGAGCAAGGTGATCAAGGGCAGCCTGCCCCTGATCGGC
GAGGCCGACTGCCTGCACGAGGAGTACGGCGGCCTGAACAAGAGCAAGCCCTACTACACCG
GCAAGCACGCCAAGGCCATCGGCAACTGCCCCATCTGGGTGAAGACCCCTCTGAAGCTGGC
CAACGGCACCAAGTACAGGCCTCCCGCCAAGCTGCTGAAGGAGAGGGGCTTCTTCGGCGCC
ATCGCCGGCTTCCTGGAGGGCGGCTGGGAGGGCATGATCGCCGGCTGGCACGGCTACACCA
GCCACGGCGCCCACGGCGTGGCCGTGGCCGCCGACCTGAAGTCCACCCAGGAGGCCATCAA
CAAGATCACCAAGAACCTGAACAGCCTGAGCGAGCTGGAGGTGAAGAACCTGCAGAGGCT
GAGCGGCGCTATGGACGAGCTGCACAACGAGATCCTGGAGCTGGACGAGAAGGTGGACGA
CCTGAGGGCCGACACCATCAGCAGCCAGATCGAGCTGGCCGTGCTGCTGAGCAACGAGGGC
ATCATCAACAGCGAGGACGAGCACCTGCTGGCCCTGGAGAGGAAGCTGAAGAAGATGCTG
GGCCCCAGCGCCGTGGACATCGGCAACGGCTGCTTCGAGACAAAGCACAAGTGCAACCAGA
CCTGCCTGGACAGGATCGCCGCCGGCACCTTCAACGCCGGCGAGTTCAGCCTGCCCACCTTC
GACAGCCTGAACATCACCGCCGCCAGCCTGAACGACGACGGCCTGGACAACCACACCATCC
TGCTGTACTACAGCACCGCCGCCAGCAGCCTGGCCGTGACCCTGATGCTGGCCATCTTCATC
GTGTATATGGTGAGCAGGGACAACGTGAGCTGCAGCATCTGCCTGTGATAGTAA
SEQ ID NO: 13 (nsP1-nsP4 of SEQ ID NOs: 1-4)
ATGGAGAAAGTTCACGTTGACATCGAGGAAGACAGCCCATTCCTCAGAGCTTTGCAGCGGA
GCTTCCCGCAGTTTGAGGTAGAAGCCAAGCAGGTCACTGATAATGACCATGCTAATGCCAG
AGCGTTTTCGCATCTGGCTTCAAAACTGATCGAAACGGAGGTGGACCCATCCGACACGATCC
TTGACATTGGAAGTGCGCCCGCCCGCAGAATGTATTCTAAGCACAAGTATCATTGTATCTGT
CCGATGAGATGTGCGGAAGATCCGGACAGATTGTATAAGTATGCAACTAAGCTGAAGAAAA
ACTGTAAGGAAATAACTGATAAGGAATTGGACAAGAAAATGAAGGAGCTGGCCGCCGTCAT
GAGCGACCCTGACCTGGAAACTGAGACTATGTGCCTCCACGACGACGAGTCGTGTCGCTAC
GAAGGGCAAGTCGCTGTTTACCAGGATGTATACGCCGTCGACGGCCCCACCAGCCTGTACC
ACCAGGCCAACAAGGGCGTGAGGGTGGCCTACTGGATCGGCTTCGACACCACACCCTTCAT
GTTCAAGAACCTGGCCGGCGCCTACCCCAGCTACAGCACCAACTGGGCCGACGAGACAGTG
CTGACCGCCAGGAACATCGGCCTGTGCAGCAGCGACGTGATGGAGAGGAGCCGGAGGGGC
ATGAGCATCCTGAGGAAGAAGTACCTGAAGCCCAGCAACAACGTGCTGTTCAGCGTGGGCA
GCACCATCTACCACGAGAAGAGGGACCTGCTGAGGAGCTGGCACCTGCCCAGCGTGTTCCA
CCTGAGGGGCAAGCAGAACTACACCTGCAGGTGCGAGACAATCGTGAGCTGCGACGGCTAC
GTGGTGAAGAGGATCGCCATCAGCCCCGGCCTGTACGGCAAGCCCAGCGGCTACGCCGCCA
CCATGCACAGGGAGGGCTTCCTGTGCTGCAAGGTGACCGACACCCTGAACGGCGAGAGGGT
GAGCTTCCCCGTGTGCACCTACGTGCCCGCCACCCTGTGCGACCAGATGACCGGCATCCTGG
CCACCGACGTGAGCGCCGACGACGCCCAGAAGCTGCTGGTGGGCCTGAACCAGAGGATCGT
GGTGAACGGCAGGACCCAGAGGAACACCAACACCATGAAGAACTACCTGCTGCCCGTGGTG
GCCCAGGCCTTCGCCAGGTGGGCCAAGGAGTACAAGGAGGACCAGGAGGACGAGAGGCCC
CTGGGCCTGAGGGACCGACAGCTGGTGATGGGCTGCTGCTGGGCCTTCAGGCGGCACAAGA
TCACCAGCATCTACAAGAGGCCCGACACCCAGACCATCATCAAGGTGAACAGCGACTTCCA
CAGCTTCGTGCTGCCCAGGATCGGCAGCAACACCCTGGAGATCGGCCTGAGGACCCGGATC
AGGAAGATGCTGGAGGAGCACAAGGAGCCCAGCCCTCTGATCACCGCCGAGGACGTGCAG
GAGGCCAAGTGCGCCGCCGACGAGGCCAAGGAGGTGAGGGAGGCCGAGGAGCTGAGGGCC
GCCCTGCCTCCCCTGGCCGCCGACGTGGAGGAGCCCACCCTGGAGGCCGACGTGGACCTGA
TGCTGCAGGAGGCCGGCGCCGGCAGCGTGGAGACACCCAGGGGCCTGATCAAGGTGACCA
GCTACGACGGCGAGGACAAGATCGGCAGCTACGCCGTGCTCAGCCCTCAGGCCGTGCTGAA
GTCCGAGAAGCTGAGCTGCATCCACCCTCTGGCCGAGCAGGTGATCGTGATCACCCACAGC
GGCAGGAAGGGCAGGTACGCCGTGGAGCCCTACCACGGCAAGGTGGTGGTCCCCGAGGGC
CACGCCATCCCCGTGCAGGACTTCCAGGCCCTGAGCGAGAGCGCCACCATCGTGTATAACG
AGAGGGAGTTCGTGAACAGGTACCTGCACCACATCGCCACCCACGGCGGCGCCCTGAACAC
CGACGAGGAGTACTACAAGACCGTGAAGCCCAGCGAGCACGACGGCGAGTACCTGTACGA
CATCGACAGGAAGCAGTGCGTGAAGAAGGAGCTGGTGACCGGCCTGGGCCTGACCGGCGA
GCTGGTGGACCCTCCCTTCCACGAGTTCGCCTACGAGAGCCTGAGGACCAGGCCCGCCGCTC
CCTACCAGGTGCCCACCATCGGCGTGTACGGCGTGCCCGGCAGCGGCAAGAGCGGCATCAT
CAAGAGCGCCGTGACCAAGAAGGACCTGGTGGTGAGCGCCAAGAAGGAGAACTGCGCCGA
GATCATCAGGGACGTGAAGAAGATGAAGGGCCTGGACGTGAACGCCAGGACCGTGGACAG
CGTGCTCCTGAACGGCTGCAAGCACCCCGTGGAGACACTGTATATCGACGAGGCCTTCGCCT
GCCACGCCGGCACCCTGAGGGCCCTGATCGCCATCATCAGGCCCAAGAAGGCCGTGCTGTG
CGGCGACCCCAAGCAGTGCGGCTTCTTCAACATGATGTGCCTGAAGGTGCACTTCAACCACG
AGATCTGCACCCAGGTGTTCCACAAGAGCATCAGCAGGCGGTGCACCAAGAGCGTGACCAG
CGTGGTGAGCACCCTGTTCTACGACAAGAAGATGAGGACCACCAACCCCAAGGAGACAAAG
ATCGTGATCGACACCACCGGCAGCACCAAGCCCAAGCAGGACGACCTGATCCTGACCTGCT
TCAGGGGCTGGGTGAAGCAGCTGCAGATCGACTACAAGGGCAACGAGATCATGACCGCCGC
CGCTAGCCAGGGCCTGACCAGGAAGGGCGTGTACGCCGTGAGGTACAAGGTGAACGAGAA
TCCCCTGTACGCCCCTACCAGCGAGCACGTGAACGTCCTGCTGACCAGGACCGAGGACAGG
ATCGTGTGGAAGACCCTGGCCGGCGACCCCTGGATCAAGACCCTGACCGCCAAGTACCCCG
GCAACTTCACCGCCACCATCGAGGAGTGGCAGGCCGAGCACGACGCCATCATGAGGCACAT
CCTGGAGAGGCCCGACCCCACCGACGTGTTCCAGAACAAGGCCAACGTGTGCTGGGCCAAG
GCCCTGGTGCCCGTGCTGAAGACCGCCGGCATCGACATGACCACCGAGCAGTGGAACACCG
TGGACTACTTCGAGACAGACAAGGCCCACAGCGCCGAGATCGTGCTGAACCAGCTGTGCGT
GAGGTTCTTCGGCCTGGACCTGGACAGCGGCCTGTTCAGCGCCCCTACCGTGCCCCTGAGCA
TCAGGAACAACCACTGGGACAACAGCCCCAGCCCCAACATGTACGGCCTGAACAAGGAGGT
GGTGAGGCAGCTGAGCAGGCGGTACCCTCAGCTGCCCAGGGCCGTGGCCACCGGCAGGGTG
TACGACATGAACACCGGCACCCTGAGGAACTACGACCCCAGGATCAACCTGGTGCCCGTGA
ACAGGCGGCTGCCACACGCCCTGGTGCTGCACCACAACGAGCACCCTCAGAGCGACTTCAG
CAGCTTCGTGAGCAAGCTGAAGGGCAGGACCGTGCTGGTGGTGGGCGAGAAGCTGAGCGTG
CCCGGCAAGATGGTGGACTGGCTGAGCGACAGGCCCGAGGCCACCTTCCGGGCCAGGCTGG
ACCTGGGCATCCCCGGCGACGTGCCCAAGTACGACATCATCTTCGTGAACGTGAGGACCCCT
TACAAGTACCACCACTACCAGCAGTGCGAGGACCACGCCATCAAGCTGAGCATGCTGACCA
AGAAGGCCTGCCTGCACCTGAACCCCGGCGGCACCTGCGTGAGCATCGGCTACGGCTACGC
CGACAGGGCCAGCGAGAGCATCATCGGCGCCATCGCCAGGCTGTTCAAGTTCAGCAGGGTG
TGCAAGCCCAAGAGCAGCCTGGAGGAGACAGAGGTGCTGTTCGTGTTCATCGGCTACGACC
GGAAGGCCAGGACCCACAACCCCTACAAGCTGAGCAGCACCCTGACCAACATCTACACCGG
CAGCAGGCTGCACGAGGCCGGCTGCGCCCCTAGCTACCACGTGGTGAGGGGCGACATCGCC
ACCGCCACCGAGGGCGTGATCATCAACGCCGCCAACAGCAAGGGCCAGCCCGGCGGCGGG
GTGTGCGGCGCCCTGTATAAGAAGTTCCCCGAGAGCTTCGACCTGCAGCCCATCGAGGTGG
GCAAGGCCAGGCTGGTGAAGGGCGCCGCCAAGCACATCATCCACGCCGTGGGCCCCAACTT
CAACAAGGTGAGCGAGGTGGAGGGCGACAAGCAGCTGGCCGAGGCCTACGAGAGCATCGC
CAAGATCGTGAACGACAACAACTACAAGAGCGTGGCCATCCCTCTGCTGAGCACCGGCATC
TTCAGCGGCAACAAGGACAGGCTGACCCAGAGCCTGAACCACCTGCTGACCGCCCTGGACA
CCACCGACGCCGACGTGGCCATCTACTGCAGGGACAAGAAGTGGGAGATGACCCTGAAGGA
GGCCGTGGCCAGGCGGGAGGCCGTGGAGGAGATCTGCATCAGCGACGACAGCAGCGTGAC
GGAGCCCGACGCCGAGCTGGTGAGGGTGCACCCCAAGAGCAGCCTGGCCGGCAGGAAGGG
CTACAGCACCAGCGACGGCAAGACCTTCAGCTACCTGGAGGGCACCAAGTTCCACCAGGCC
GCCAAGGACATCGCCGAGATCAACGCCATGTGGCCCGTGGCCACCGAGGCCAACGAGCAG
GTGTGCATGTATATCCTGGGCGAGAGCATGAGCAGCATCAGGAGCAAGTGCCCCGTGGAGG
AGAGCGAGGCCAGCACCCCTCCCAGCACCCTGCCCTGCCTGTGCATCCACGCCATGACCCCT
GAGAGGGTGCAGCGGCTGAAGGCCAGCAGGCCCGAGCAGATCACCGTGTGCAGCAGCTTCC
CTCTGCCCAAGTACCGGATCACCGGCGTGCAGAAGATCCAGTGCAGCCAGCCCATCCTGTTC
AGCCCCAAGGTGCCCGCCTACATCCACCCCAGGAAGTACCTGGTGGAGACACCCCCCGTGG
ACGAGACACCCGAGCCCAGCGCCGAGAACCAGAGCACCGAGGGCACCCCTGAGCAGCCTC
CCCTGATCACCGAGGACGAGACAAGGACCAGGACGCCCGAGCCCATCATCATTGAGGAGGA
AGAGGAGGACAGCATCAGCCTGCTGAGCGACGGCCCCACCCACCAGGTGCTGCAGGTGGAG
GCCGACATCCACGGCCCTCCCAGCGTGAGCAGCTCCAGCTGGAGCATCCCTCACGCCAGCG
ACTTCGACGTGGACAGCCTGAGCATCCTGGACACCCTGGAGGGCGCCAGCGTGACCAGCGG
CGCCACCAGCGCCGAGACAAACAGCTACTTCGCCAAGAGCATGGAGTTCCTGGCCAGGCCC
GTGCCCGCCCCTAGGACCGTGTTCAGGAACCCTCCCCACCCCGCCCCTAGGACCAGGACCCC
TAGCCTGGCCCCTAGCAGGGCCTGCAGCAGGACCAGCCTGGTGAGCACCCCTCCCGGCGTG
AACCGGGTGATCACCAGGGAGGAGCTGGAGGCCCTGACCCCTAGCAGGACCCCTAGCAGGA
GCGTGAGCAGGACCAGCCTGGTGAGCAACCCTCCCGGCGTGAACCGGGTGATCACCAGGGA
GGAGTTCGAGGCCTTCGTGGCCCAGCAGCAAAGGCGGTTCGACGCCGGCGCCTACATCTTC
AGCAGCGACACCGGCCAGGGCCACCTGCAGCAGAAGTCCGTGAGGCAGACCGTGCTGAGC
GAGGTGGTCCTGGAGAGGACGGAGCTGGAGATCAGCTACGCCCCTAGGCTGGACCAGGAG
AAGGAGGAGCTGCTGAGGAAGAAGCTGCAGCTGAACCCCACCCCTGCCAACAGGAGCAGG
TACCAGAGCAGGAAGGTGGAGAACATGAAGGCCATCACCGCCAGGCGGATCCTGCAGGGC
CTGGGCCACTACCTGAAGGCCGAGGGCAAGGTGGAGTGCTACAGGACCCTGCACCCCGTGC
CCCTGTACTCCAGCTCCGTGAACAGGGCCTTCAGCAGCCCCAAGGTGGCCGTGGAGGCCTG
CAACGCCATGCTGAAGGAGAACTTCCCCACCGTGGCCAGCTACTGCATCATCCCCGAGTAC
GACGCCTACCTGGACATGGTGGACGGCGCCAGCTGCTGCCTGGACACCGCCAGCTTCTGCC
CCGCCAAGCTGAGGAGCTTCCCCAAGAAGCACAGCTACCTGGAGCCCACCATCAGGAGCGC
CGTGCCCAGCGCCATCCAGAACACCCTGCAGAACGTGCTGGCCGCCGCTACCAAGAGGAAC
TGCAACGTGACCCAGATGAGGGAGCTGCCCGTGCTGGACAGCGCCGCCTTCAACGTGGAGT
GCTTCAAGAAGTACGCCTGCAACAACGAGTACTGGGAGACATTCAAGGAGAACCCCATCAG
GCTGACCGAGGAGAACGTGGTGAACTACATCACCAAGCTGAAGGGCCCCAAGGCCGCCGCT
CTGTTCGCCAAGACCCACAACCTGAACATGCTCCAGGACATCCCTATGGACAGGTTCGTGAT
GGACCTGAAGAGGGACGTGAAGGTGACCCCTGGCACCAAGCACACCGAGGAGAGGCCCAA
GGTGCAGGTGATCCAGGCCGCCGACCCTCTGGCCACCGCCTACCTGTGCGGCATCCACAGG
GAGCTGGTGAGGCGGCTGAACGCCGTCCTGCTGCCCAACATCCACACCCTGTTCGACATGA
GCGCCGAGGACTTCGACGCCATCATCGCCGAGCACTTCCAGCCCGGCGACTGCGTGCTGGA
GACAGACATCGCCAGCTTCGACAAGAGCGAGGACGACGCTATGGCCCTGACCGCCCTGATG
ATCCTGGAGGACCTGGGCGTGGACGCCGAGCTGCTGACCCTGATCGAGGCCGCCTTCGGCG
AGATCAGCAGCATCCACCTGCCCACCAAGACCAAGTTCAAGTTCGGCGCCATGATGAAGTC
CGGCATGTTCCTGACCCTGTTCGTGAACACCGTGATCAACATCGTGATCGCCAGCAGGGTGC
TGCGGGAGAGGCTGACCGGCAGCCCCTGCGCCGCCTTCATCGGCGACGACAACATCGTGAA
GGGCGTGAAGTCCGACAAGCTGATGGCCGACAGGTGCGCCACCTGGCTGAACATGGAGGTG
AAGATCATCGACGCCGTGGTGGGCGAGAAGGCCCCTTACTTCTGCGGCGGCTTCATCCTGTG
CGACAGCGTGACCGGCACCGCCTGCAGGGTGGCCGACCCTCTGAAGAGGCTGTTCAAGCTG
GGCAAGCCCCTGGCCGCCGACGACGAGCACGACGACGATAGGCGGAGGGCCCTGCACGAG
GAGAGCACCAGGTGGAACCGGGTGGGCATCCTGAGCGAGCTGTGCAAGGCCGTGGAGAGC
AGGTACGAGACAGTGGGCACCAGCATCATCGTGATGGCCATGACCACCCTGGCCAGCAGCG
TCAAGTCCTTCAGCTACCTGAGGGGGGCCCCTATAACTCTCTACGGCTAA
SEQ ID NO: 14 (5′ UTR of SEQ ID NOs. 1-4)
ATGGGCGGCGCATGAGAGAAGCCCAGACCAATTACCTACCCAAA
SEQ ID NO: 15 (3′ UTR of SEQ ID NOs. 1-4)
ACTCGAGTATGTTACGTGCAAAGGTGATTGTCACCCCCCGAAAGACCATATTGTGACACACC
CTCAGTATCACGCCCAAACATTTACAGCCGCGGTGTCAAAAACCGCGTGGACGTGGTTAAC
ATCCCTGCTGGGAGGATCAGCCGTAATTATTATAATTGGCTTGGTGCTGGCTACTATTGTGG
CCATGTACGTGCTGACCAACCAGAAACATAATTGAATACAGCAGCAATTGGCAAGCTGCTT
ACATAGAACTCGCGGCGATTGGCATGCCGCCTTAAAATTTTTATTTTATTTTTTCTTTTCTTTT
CCGAATCGGATTTTGTTTTTAATATTTC
SEQ ID NO: 16 (Example sequence encoding a poly-A tail)
AAAAAAAAAAAAAAAAAAAAAAAAATCTAGAAAAAAAAAAAAAAAAAAAAAAAAAAAAA
AAAAAAAAAAAAAAAAAAAAAAAAAAAAAAAAAAAAAAAAAAAAAAAAAAAAAAAAAA
AAAAAAAAAAAAA
SEQ ID NO: 18 (3′ UTR (Xbg), with poly-A)
ACTCGAGCTAGTGACTGACTAGGATCTGGTTACCACTAAACCAGCCTCAAGAACACCCGAA
TGGAGTCTCTAAGCTACATAATACCAACTTACACTTACAAAATGTTGTCCCCCAAAATGTAG
CCATTCGTATCTGCTCCTAATAAAAAGAAAGTTTCTTCACATTCTAGAAAAAAAAAAAAAA
AAAAAAAAAAAAAAAAAAAAAAAAAAAAAAAAAAAAAAAAAAAAAAAAAAAAAAAAAA
AAAAAAAAAAAAAAAAAAAAAAAAAAAA
SEQ ID NO: 19 (3′ UTR (Xbg), without poly-A)
ACTCGAGCTAGTGACTGACTAGGATCTGGTTACCACTAAACCAGCCTCAAGAACACCCGAA
TGGAGTCTCTAAGCTACATAATACCAACTTACACTTACAAAATGTTGTCCCCCAAAATGTAG
CCATTCGTATCTGCTCCTAATAAAAAGAAAGTTTCTTCACATTCTAG
SEQ ID NO: 20 (5′ UTR (alternative VEEV-derived sequence))
GATGGGCGGCGCATGAGAGAAGCCCAGACCAATTACCTACCCAAA
SEQ ID NO: 21 (5′ UTR (alternative VEEV-derived sequence))
GATAGGCGGCGCATGAGAGAAGCCCAGACCAATTACCTACCCAAA
SEQ ID NO: 150 (intergenic sequence)
ACTCGAAGCAGTGTTAAATCATTCAGCTACCTGAGAGGGGCCCCTATAACTCTCTACGGCTA
ACCTGAATGGACTACGACATAGTCTAGTCCGCCAAGGCCGCCACC
SEQ ID NO: 151 (sgP NA sgP CVB3 HA)
ATGGGCGGCGCATGAGAGAAGCCCAGACCAATTACCTACCCAAAATGGAGAAAGTTCACGT
TGACATCGAGGAAGACAGCCCATTCCTCAGAGCTTTGCAGCGGAGCTTCCCGCAGTTTGAG
GTAGAAGCCAAGCAGGTCACTGATAATGACCATGCTAATGCCAGAGCGTTTTCGCATCTGG
CTTCAAAACTGATCGAAACGGAGGTGGACCCATCCGACACGATCCTTGACATTGGAAGTGC
GCCCGCCCGCAGAATGTATTCTAAGCACAAGTATCATTGTATCTGTCCGATGAGATGTGCGG
AAGATCCGGACAGATTGTATAAGTATGCAACTAAGCTGAAGAAAAACTGTAAGGAAATAAC
TGATAAGGAATTGGACAAGAAAATGAAGGAGCTGGCCGCCGTCATGAGCGACCCTGACCTG
GAAACTGAGACTATGTGCCTCCACGACGACGAGTCGTGTCGCTACGAAGGGCAAGTCGCTG
TTTACCAGGATGTATACGCCGTCGACGGCCCCACCAGCCTGTACCACCAGGCCAACAAGGG
CGTGAGGGTGGCCTACTGGATCGGCTTCGACACCACACCCTTCATGTTCAAGAACCTGGCCG
GCGCCTACCCCAGCTACAGCACCAACTGGGCCGACGAGACAGTGCTGACCGCCAGGAACAT
CGGCCTGTGCAGCAGCGACGTGATGGAGAGGAGCCGGAGGGGCATGAGCATCCTGAGGAA
GAAGTACCTGAAGCCCAGCAACAACGTGCTGTTCAGCGTGGGCAGCACCATCTACCACGAG
AAGAGGGACCTGCTGAGGAGCTGGCACCTGCCCAGCGTGTTCCACCTGAGGGGCAAGCAGA
ACTACACCTGCAGGTGCGAGACAATCGTGAGCTGCGACGGCTACGTGGTGAAGAGGATCGC
CATCAGCCCCGGCCTGTACGGCAAGCCCAGCGGCTACGCCGCCACCATGCACAGGGAGGGC
TTCCTGTGCTGCAAGGTGACCGACACCCTGAACGGCGAGAGGGTGAGCTTCCCCGTGTGCA
CCTACGTGCCCGCCACCCTGTGCGACCAGATGACCGGCATCCTGGCCACCGACGTGAGCGC
CGACGACGCCCAGAAGCTGCTGGTGGGCCTGAACCAGAGGATCGTGGTGAACGGCAGGACC
CAGAGGAACACCAACACCATGAAGAACTACCTGCTGCCCGTGGTGGCCCAGGCCTTCGCCA
GGTGGGCCAAGGAGTACAAGGAGGACCAGGAGGACGAGAGGCCCCTGGGCCTGAGGGACC
GACAGCTGGTGATGGGCTGCTGCTGGGCCTTCAGGCGGCACAAGATCACCAGCATCTACAA
GAGGCCCGACACCCAGACCATCATCAAGGTGAACAGCGACTTCCACAGCTTCGTGCTGCCC
AGGATCGGCAGCAACACCCTGGAGATCGGCCTGAGGACCCGGATCAGGAAGATGCTGGAG
GAGCACAAGGAGCCCAGCCCTCTGATCACCGCCGAGGACGTGCAGGAGGCCAAGTGCGCC
GCCGACGAGGCCAAGGAGGTGAGGGAGGCCGAGGAGCTGAGGGCCGCCCTGCCTCCCCTG
GCCGCCGACGTGGAGGAGCCCACCCTGGAGGCCGACGTGGACCTGATGCTGCAGGAGGCCG
GCGCCGGCAGCGTGGAGACACCCAGGGGCCTGATCAAGGTGACCAGCTACGACGGCGAGG
ACAAGATCGGCAGCTACGCCGTGCTCAGCCCTCAGGCCGTGCTGAAGTCCGAGAAGCTGAG
CTGCATCCACCCTCTGGCCGAGCAGGTGATCGTGATCACCCACAGCGGCAGGAAGGGCAGG
TACGCCGTGGAGCCCTACCACGGCAAGGTGGTGGTCCCCGAGGGCCACGCCATCCCCGTGC
AGGACTTCCAGGCCCTGAGCGAGAGCGCCACCATCGTGTATAACGAGAGGGAGTTCGTGAA
CAGGTACCTGCACCACATCGCCACCCACGGCGGCGCCCTGAACACCGACGAGGAGTACTAC
AAGACCGTGAAGCCCAGCGAGCACGACGGCGAGTACCTGTACGACATCGACAGGAAGCAG
TGCGTGAAGAAGGAGCTGGTGACCGGCCTGGGCCTGACCGGCGAGCTGGTGGACCCTCCCT
TCCACGAGTTCGCCTACGAGAGCCTGAGGACCAGGCCCGCCGCTCCCTACCAGGTGCCCAC
CATCGGCGTGTACGGCGTGCCCGGCAGCGGCAAGAGCGGCATCATCAAGAGCGCCGTGACC
AAGAAGGACCTGGTGGTGAGCGCCAAGAAGGAGAACTGCGCCGAGATCATCAGGGACGTG
AAGAAGATGAAGGGCCTGGACGTGAACGCCAGGACCGTGGACAGCGTGCTCCTGAACGGCT
GCAAGCACCCCGTGGAGACACTGTATATCGACGAGGCCTTCGCCTGCCACGCCGGCACCCT
GAGGGCCCTGATCGCCATCATCAGGCCCAAGAAGGCCGTGCTGTGCGGCGACCCCAAGCAG
TGCGGCTTCTTCAACATGATGTGCCTGAAGGTGCACTTCAACCACGAGATCTGCACCCAGGT
GTTCCACAAGAGCATCAGCAGGCGGTGCACCAAGAGCGTGACCAGCGTGGTGAGCACCCTG
TTCTACGACAAGAAGATGAGGACCACCAACCCCAAGGAGACAAAGATCGTGATCGACACCA
CCGGCAGCACCAAGCCCAAGCAGGACGACCTGATCCTGACCTGCTTCAGGGGCTGGGTGAA
GCAGCTGCAGATCGACTACAAGGGCAACGAGATCATGACCGCCGCCGCTAGCCAGGGCCTG
ACCAGGAAGGGCGTGTACGCCGTGAGGTACAAGGTGAACGAGAATCCCCTGTACGCCCCTA
CCAGCGAGCACGTGAACGTCCTGCTGACCAGGACCGAGGACAGGATCGTGTGGAAGACCCT
GGCCGGCGACCCCTGGATCAAGACCCTGACCGCCAAGTACCCCGGCAACTTCACCGCCACC
ATCGAGGAGTGGCAGGCCGAGCACGACGCCATCATGAGGCACATCCTGGAGAGGCCCGACC
CCACCGACGTGTTCCAGAACAAGGCCAACGTGTGCTGGGCCAAGGCCCTGGTGCCCGTGCT
GAAGACCGCCGGCATCGACATGACCACCGAGCAGTGGAACACCGTGGACTACTTCGAGACA
GACAAGGCCCACAGCGCCGAGATCGTGCTGAACCAGCTGTGCGTGAGGTTCTTCGGCCTGG
ACCTGGACAGCGGCCTGTTCAGCGCCCCTACCGTGCCCCTGAGCATCAGGAACAACCACTG
GGACAACAGCCCCAGCCCCAACATGTACGGCCTGAACAAGGAGGTGGTGAGGCAGCTGAG
CAGGCGGTACCCTCAGCTGCCCAGGGCCGTGGCCACCGGCAGGGTGTACGACATGAACACC
GGCACCCTGAGGAACTACGACCCCAGGATCAACCTGGTGCCCGTGAACAGGCGGCTGCCAC
ACGCCCTGGTGCTGCACCACAACGAGCACCCTCAGAGCGACTTCAGCAGCTTCGTGAGCAA
GCTGAAGGGCAGGACCGTGCTGGTGGTGGGCGAGAAGCTGAGCGTGCCCGGCAAGATGGT
GGACTGGCTGAGCGACAGGCCCGAGGCCACCTTCCGGGCCAGGCTGGACCTGGGCATCCCC
GGCGACGTGCCCAAGTACGACATCATCTTCGTGAACGTGAGGACCCCTTACAAGTACCACC
ACTACCAGCAGTGCGAGGACCACGCCATCAAGCTGAGCATGCTGACCAAGAAGGCCTGCCT
GCACCTGAACCCCGGCGGCACCTGCGTGAGCATCGGCTACGGCTACGCCGACAGGGCCAGC
GAGAGCATCATCGGCGCCATCGCCAGGCTGTTCAAGTTCAGCAGGGTGTGCAAGCCCAAGA
GCAGCCTGGAGGAGACAGAGGTGCTGTTCGTGTTCATCGGCTACGACCGGAAGGCCAGGAC
CCACAACCCCTACAAGCTGAGCAGCACCCTGACCAACATCTACACCGGCAGCAGGCTGCAC
GAGGCCGGCTGCGCCCCTAGCTACCACGTGGTGAGGGGCGACATCGCCACCGCCACCGAGG
GCGTGATCATCAACGCCGCCAACAGCAAGGGCCAGCCCGGCGGCGGGGTGTGCGGCGCCCT
GTATAAGAAGTTCCCCGAGAGCTTCGACCTGCAGCCCATCGAGGTGGGCAAGGCCAGGCTG
GTGAAGGGCGCCGCCAAGCACATCATCCACGCCGTGGGCCCCAACTTCAACAAGGTGAGCG
AGGTGGAGGGCGACAAGCAGCTGGCCGAGGCCTACGAGAGCATCGCCAAGATCGTGAACG
ACAACAACTACAAGAGCGTGGCCATCCCTCTGCTGAGCACCGGCATCTTCAGCGGCAACAA
GGACAGGCTGACCCAGAGCCTGAACCACCTGCTGACCGCCCTGGACACCACCGACGCCGAC
GTGGCCATCTACTGCAGGGACAAGAAGTGGGAGATGACCCTGAAGGAGGCCGTGGCCAGG
CGGGAGGCCGTGGAGGAGATCTGCATCAGCGACGACAGCAGCGTGACGGAGCCCGACGCC
GAGCTGGTGAGGGTGCACCCCAAGAGCAGCCTGGCCGGCAGGAAGGGCTACAGCACCAGC
GACGGCAAGACCTTCAGCTACCTGGAGGGCACCAAGTTCCACCAGGCCGCCAAGGACATCG
CCGAGATCAACGCCATGTGGCCCGTGGCCACCGAGGCCAACGAGCAGGTGTGCATGTATAT
CCTGGGCGAGAGCATGAGCAGCATCAGGAGCAAGTGCCCCGTGGAGGAGAGCGAGGCCAG
CACCCCTCCCAGCACCCTGCCCTGCCTGTGCATCCACGCCATGACCCCTGAGAGGGTGCAGC
GGCTGAAGGCCAGCAGGCCCGAGCAGATCACCGTGTGCAGCAGCTTCCCTCTGCCCAAGTA
CCGGATCACCGGCGTGCAGAAGATCCAGTGCAGCCAGCCCATCCTGTTCAGCCCCAAGGTG
CCCGCCTACATCCACCCCAGGAAGTACCTGGTGGAGACACCCCCCGTGGACGAGACACCCG
AGCCCAGCGCCGAGAACCAGAGCACCGAGGGCACCCCTGAGCAGCCTCCCCTGATCACCGA
GGACGAGACAAGGACCAGGACGCCCGAGCCCATCATCATTGAGGAGGAAGAGGAGGACAG
CATCAGCCTGCTGAGCGACGGCCCCACCCACCAGGTGCTGCAGGTGGAGGCCGACATCCAC
GGCCCTCCCAGCGTGAGCAGCTCCAGCTGGAGCATCCCTCACGCCAGCGACTTCGACGTGG
ACAGCCTGAGCATCCTGGACACCCTGGAGGGCGCCAGCGTGACCAGCGGCGCCACCAGCGC
CGAGACAAACAGCTACTTCGCCAAGAGCATGGAGTTCCTGGCCAGGCCCGTGCCCGCCCCT
AGGACCGTGTTCAGGAACCCTCCCCACCCCGCCCCTAGGACCAGGACCCCTAGCCTGGCCC
CTAGCAGGGCCTGCAGCAGGACCAGCCTGGTGAGCACCCCTCCCGGCGTGAACCGGGTGAT
CACCAGGGAGGAGCTGGAGGCCCTGACCCCTAGCAGGACCCCTAGCAGGAGCGTGAGCAG
GACCAGCCTGGTGAGCAACCCTCCCGGCGTGAACCGGGTGATCACCAGGGAGGAGTTCGAG
GCCTTCGTGGCCCAGCAGCAAAGGCGGTTCGACGCCGGCGCCTACATCTTCAGCAGCGACA
CCGGCCAGGGCCACCTGCAGCAGAAGTCCGTGAGGCAGACCGTGCTGAGCGAGGTGGTCCT
GGAGAGGACGGAGCTGGAGATCAGCTACGCCCCTAGGCTGGACCAGGAGAAGGAGGAGCT
GCTGAGGAAGAAGCTGCAGCTGAACCCCACCCCTGCCAACAGGAGCAGGTACCAGAGCAG
GAAGGTGGAGAACATGAAGGCCATCACCGCCAGGCGGATCCTGCAGGGCCTGGGCCACTAC
CTGAAGGCCGAGGGCAAGGTGGAGTGCTACAGGACCCTGCACCCCGTGCCCCTGTACTCCA
GCTCCGTGAACAGGGCCTTCAGCAGCCCCAAGGTGGCCGTGGAGGCCTGCAACGCCATGCT
GAAGGAGAACTTCCCCACCGTGGCCAGCTACTGCATCATCCCCGAGTACGACGCCTACCTG
GACATGGTGGACGGCGCCAGCTGCTGCCTGGACACCGCCAGCTTCTGCCCCGCCAAGCTGA
GGAGCTTCCCCAAGAAGCACAGCTACCTGGAGCCCACCATCAGGAGCGCCGTGCCCAGCGC
CATCCAGAACACCCTGCAGAACGTGCTGGCCGCCGCTACCAAGAGGAACTGCAACGTGACC
CAGATGAGGGAGCTGCCCGTGCTGGACAGCGCCGCCTTCAACGTGGAGTGCTTCAAGAAGT
ACGCCTGCAACAACGAGTACTGGGAGACATTCAAGGAGAACCCCATCAGGCTGACCGAGGA
GAACGTGGTGAACTACATCACCAAGCTGAAGGGCCCCAAGGCCGCCGCTCTGTTCGCCAAG
ACCCACAACCTGAACATGCTCCAGGACATCCCTATGGACAGGTTCGTGATGGACCTGAAGA
GGGACGTGAAGGTGACCCCTGGCACCAAGCACACCGAGGAGAGGCCCAAGGTGCAGGTGA
TCCAGGCCGCCGACCCTCTGGCCACCGCCTACCTGTGCGGCATCCACAGGGAGCTGGTGAG
GCGGCTGAACGCCGTCCTGCTGCCCAACATCCACACCCTGTTCGACATGAGCGCCGAGGAC
TTCGACGCCATCATCGCCGAGCACTTCCAGCCCGGCGACTGCGTGCTGGAGACAGACATCG
CCAGCTTCGACAAGAGCGAGGACGACGCTATGGCCCTGACCGCCCTGATGATCCTGGAGGA
CCTGGGCGTGGACGCCGAGCTGCTGACCCTGATCGAGGCCGCCTTCGGCGAGATCAGCAGC
ATCCACCTGCCCACCAAGACCAAGTTCAAGTTCGGCGCCATGATGAAGTCCGGCATGTTCCT
GACCCTGTTCGTGAACACCGTGATCAACATCGTGATCGCCAGCAGGGTGCTGCGGGAGAGG
CTGACCGGCAGCCCCTGCGCCGCCTTCATCGGCGACGACAACATCGTGAAGGGCGTGAAGT
CCGACAAGCTGATGGCCGACAGGTGCGCCACCTGGCTGAACATGGAGGTGAAGATCATCGA
CGCCGTGGTGGGCGAGAAGGCCCCTTACTTCTGCGGCGGCTTCATCCTGTGCGACAGCGTGA
CCGGCACCGCCTGCAGGGTGGCCGACCCTCTGAAGAGGCTGTTCAAGCTGGGCAAGCCCCT
GGCCGCCGACGACGAGCACGACGACGATAGGCGGAGGGCCCTGCACGAGGAGAGCACCAG
GTGGAACCGGGTGGGCATCCTGAGCGAGCTGTGCAAGGCCGTGGAGAGCAGGTACGAGAC
AGTGGGCACCAGCATCATCGTGATGGCCATGACCACCCTGGCCAGCAGCGTCAAGTCCTTC
AGCTACCTGAGGGGGGCCCCTATAACTCTCTACGGCTAACCTGAATGGACTACGACATAGTC
TAGTCCGCCAAGGCCGCCACCATGAATCCTAACCAGAAGATCATCACCATCGGCAGCGTGA
GCCTGACCATCAGCACCATCTGCTTCTTCATGCAGATCGCCATCCTGATCACCACCGTGACC
CTGCACTTCAAGCAGTACGAGTTCAACAGCCCTCCCAACAACCAGGTGATGCTGTGCGAGC
CCACCATCATCGAGAGGAACATGACCGAGATCGTGTACCTGACCAACACCACCATCGAGAA
GGAGATCTGCCCCAAGCCCGCCGAGTACAGGAACTGGAGCAAGCCCCAGTGCGGCATCACC
GGCTTCGCCCCTTTCAGCAAGGACAACAGCATCAGGCTGAGCGCCGGCGGCGACATCTGGG
TGACCAGGGAGCCCTACGTGAGCTGCGACCTGGACAAGTGCTACCAGTTCGCCCTGGGCCA
GGGCACCACCCTGAACAACGTGCACAGCAACAACACCGTGAGGGACAGGACCCCTTACCGG
ACCCTGCTGATGAACGAGCTGGGCGTGCCCTTCCACCTGGGCACCAAGCAGGTGTGCATCG
CCTGGAGCAGCTCCAGCTGCCACGACGGCAAGGCCTGGCTGCACGTGTGCATCACCGGCGA
CGACAAGAACGCCACCGCCAGCTTCATCTACAACGGCAGGCTGGTGGACAGCGTGGTGAGC
TGGAGCAACGACATCCTGAGGACCCAGGAGAGCGAGTGCGTGTGCATCAACGGCACCTGCA
CCGTGGTGATGACCGACGGCAACGCCACCGGCAAGGCCGACACCAAGATCCTGTTCATCGA
GGAGGGCAAGATCGTGCACACCAGCAAGCTGAGCGGCAGCGCCCAGCACGTGGAGGAGTG
CAGCTGCTACCCCAGGTACCCCGGCGTGAGGTGCGTGTGCAGGGACAACTGGAAGGGCAGC
AACCGGCCCATCATCGACATCAACATCAAGGACCACAGCATCGTCAGCAGGTACGTGTGCA
GCGGCCTGGTGGGCGACACCCCTAGAAAGAGCGACAGCAGCTCCAGCAGCCACTGCCTGAA
CCCCAACAACGAGAAGGGCGACCACGGCGTGAAGGGCTGGGCCTTCGACGACGGCAACGA
CGTGTGGATGGGCAGGACCATCAACGAGACAAGCAGGCTGGGCTACGAGACATTCAAGGTG
GTGGAGGGCTGGAGCAACCCCAAGAGCAAGCTGCAGATCAACAGGCAGGTGATCGTGGAC
AGGGGCGACAGGAGCGGCTACAGCGGCATCTTCAGCGTGGAGGGCAAGAGCTGCATCAAC
CGGTGCTTCTACGTGGAGCTGATCAGGGGCAGGAAGGAGGAGACAGAGGTGCTGTGGACCA
GCAACAGCATCGTGGTGTTCTGCGGCACCAGCGGCACCTACGGCACCGGCAGCTGGCCCGA
CGGCGCCAACCTGAGCCTGATGCACATCTGATAGTAAACTCGACCGGAAACGCAATAGCCG
AAAAACAAAAAACAAAAAAAACAAAAAAAAAACCAAAAAAACAAAACACATTAAAACAG
CCTGTGGGTTGATCCCACCCACAGGCCCATTGGGCGCTAGCACTCTGGTATCACGGTACCTT
TGTGCGCCTGTTTTATACCCCCTCCCCCAACTGTAACTTAGAAGTAACACACACCGATCAAC
AGTCAGCGTGGCACACCAGCCACGTTTTGATCAAGCACTTCTGTTACCCCGGACTGAGTATC
AATAGACTGCTCACGCGGTTGAAGGAGAAAGCGTTCGTTATCCGGCCAACTACTTCGAAAA
ACCTAGTAACACCGTGGAAGTTGCAGAGTGTTTCGCTCAGCACTACCCCAGTGTAGATCAGG
TCGATGAGTCACCGCATTCCCCACGGGCGACCGTGGCGGTGGCTGCGTTGGCGGCCTGCCCA
TGGGGAAACCCATGGGACGCTCTAATACAGACATGGTGCGAAGAGTCTATTGAGCTAGTTG
GTAGTCCTCCGGCCCCTGAATGCGGCTAATCCTAACTGCGGAGCACACACCCTCAAGCCAG
AGGGCAGTGTGTCGTAACGGGCAACTCTGCAGCGGAACCGACTACTTTGGGTGTCCGTGTTT
CATTTTATTCCTATACTGGCTGCTTATGGTGACAATTGAGAGATCGTTACCATATAGCTATTG
GATTGGCCATCCGGTGACTAATAGAGCTATTATATATCCCTTTGTTGGGTTTATACCACTTAG
CTTGAAAGAGGTTAAAACATTACAATTCATTGTTAAGTTGAATACAGCAAAATGAAGACCA
TCATCGCCCTGAGCTACATCCTGTGCCTGGTGTTCGCCCAGAAGATCCCCGGCAATGACAAC
AGCACCGCCACCCTGTGCCTGGGCCACCACGCCGTGCCCAACGGCACCATCGTGAAGACCA
TCACCAACGACAGGATCGAGGTGACCAACGCCACCGAGCTGGTGCAGAACAGCAGCATCGG
CGAGATCTGCGACAGCCCTCACCAGATCCTGGACGGCGGCAACTGCACCCTGATCGACGCC
CTGCTGGGCGACCCTCAGTGCGACGGCTTCCAGAACAAGGAGTGGGACCTGTTCGTGGAGA
GGAGCAGGGCCAACAGCAACTGCTACCCCTACGACGTGCCCGACTACGCCAGCCTGAGGAG
CCTGGTGGCCAGCAGCGGCACCCTGGAGTTCAAGAACGAGAGCTTCAACTGGACCGGCGTG
AAGCAGAACGGCACCAGCAGCGCCTGCATCAGGGGCAGCAGCTCCAGCTTCTTCAGCCGGC
TGAATTGGCTCACCCACCTGAACTACACCTACCCCGCCCTGAACGTGACCATGCCCAACAAC
GAGCAGTTCGACAAGCTGTATATCTGGGGCGTGCACCACCCCAGCACCGACAAGGACCAGA
TCAGCCTGTTCGCCCAGCCCAGCGGCAGGATCACCGTGAGCACCAAGAGGAGCCAGCAGGC
CGTGATCCCCAACATCGGCAGCAGGCCCAGGATCAGGGACATCCCCAGCAGGATCAGCATC
TACTGGACCATCGTGAAGCCCGGCGACATCCTGCTGATCAACAGCACCGGCAACCTGATCG
CCCCTAGGGGCTACTTCAAGATCAGGAGCGGCAAGAGCAGCATCATGAGGAGCGACGCCCC
TATCGGCAAGTGCAAGAGCGAGTGCATCACCCCTAACGGCAGCATCCCCAACGACAAGCCC
TTCCAGAACGTGAACAGGATCACCTACGGCGCCTGCCCCAGGTACGTGAAGCAGAGCACCC
TGAAGCTGGCCACCGGCATGAGGAACGTGCCCGAGAAGCAGACCAGGGGCATCTTCGGCGC
CATCGCCGGCTTCATCGAGAACGGCTGGGAGGGCATGGTGGACGGCTGGTACGGCTTCAGG
CACCAGAACAGCGAGGGCAGGGGCCAGGCCGCCGACCTGAAGTCCACCCAGGCCGCCATC
GACCAGATCAACGGCAAGCTGAACAGGCTGATCGGCAAGACCAACGAGAAGTTCCACCAG
ATCGAGAAGGAGTTCAGCGAGGTGGAGGGCAGGGTGCAGGACCTGGAGAAGTACGTGGAG
GACACCAAGATCGACCTGTGGAGCTACAACGCCGAGCTGCTGGTGGCCCTGGAGAACCAGC
ACACCATCGACCTGACCGACAGCGAGATGAACAAGCTGTTCGAGAAGACCAAGAAGCAGCT
GAGGGAGAACGCCGAGGACATGGGCAACGGCTGCTTCAAGATCTACCACAAGTGCGACAA
CGCCTGCATCGGCAGCATCAGGAACGAGACATATGACCACAACGTGTACCGGGACGAGGCC
CTGAACAACCGGTTCCAGATCAAGGGCGTGGAGCTGAAGTCCGGCTACAAGGACTGGATCC
TGTGGATCAGCTTCGCCATGAGCTGCTTCCTGCTGTGCATCGCCCTGCTGGGCTTCATCATGT
GGGCCTGCCAGAAGGGCAACATCAGGTGCAACATCTGCATCTGATAGTAAACTCGAGTATG
TTACGTGCAAAGGTGATTGTCACCCCCCGAAAGACCATATTGTGACACACCCTCAGTATCAC
GCCCAAACATTTACAGCCGCGGTGTCAAAAACCGCGTGGACGTGGTTAACATCCCTGCTGG
GAGGATCAGCCGTAATTATTATAATTGGCTTGGTGCTGGCTACTATTGTGGCCATGTACGTG
CTGACCAACCAGAAACATAATTGAATACAGCAGCAATTGGCAAGCTGCTTACATAGAACTC
GCGGCGATTGGCATGCCGCCTTAAAATTTTTATTTTATTTTTTCTTTTCTTTTCCGAATCGGAT
TTTGTTTTTAATATTTC
SEQ ID NO: 152 (sgP EMCV NA sgP CVB3 HA)
ATGGGCGGCGCATGAGAGAAGCCCAGACCAATTACCTACCCAAAATGGAGAAAGTTCACGT
TGACATCGAGGAAGACAGCCCATTCCTCAGAGCTTTGCAGCGGAGCTTCCCGCAGTTTGAG
GTAGAAGCCAAGCAGGTCACTGATAATGACCATGCTAATGCCAGAGCGTTTTCGCATCTGG
CTTCAAAACTGATCGAAACGGAGGTGGACCCATCCGACACGATCCTTGACATTGGAAGTGC
GCCCGCCCGCAGAATGTATTCTAAGCACAAGTATCATTGTATCTGTCCGATGAGATGTGCGG
AAGATCCGGACAGATTGTATAAGTATGCAACTAAGCTGAAGAAAAACTGTAAGGAAATAAC
TGATAAGGAATTGGACAAGAAAATGAAGGAGCTGGCCGCCGTCATGAGCGACCCTGACCTG
GAAACTGAGACTATGTGCCTCCACGACGACGAGTCGTGTCGCTACGAAGGGCAAGTCGCTG
TTTACCAGGATGTATACGCCGTCGACGGCCCCACCAGCCTGTACCACCAGGCCAACAAGGG
CGTGAGGGTGGCCTACTGGATCGGCTTCGACACCACACCCTTCATGTTCAAGAACCTGGCCG
GCGCCTACCCCAGCTACAGCACCAACTGGGCCGACGAGACAGTGCTGACCGCCAGGAACAT
CGGCCTGTGCAGCAGCGACGTGATGGAGAGGAGCCGGAGGGGCATGAGCATCCTGAGGAA
GAAGTACCTGAAGCCCAGCAACAACGTGCTGTTCAGCGTGGGCAGCACCATCTACCACGAG
AAGAGGGACCTGCTGAGGAGCTGGCACCTGCCCAGCGTGTTCCACCTGAGGGGCAAGCAGA
ACTACACCTGCAGGTGCGAGACAATCGTGAGCTGCGACGGCTACGTGGTGAAGAGGATCGC
CATCAGCCCCGGCCTGTACGGCAAGCCCAGCGGCTACGCCGCCACCATGCACAGGGAGGGC
TTCCTGTGCTGCAAGGTGACCGACACCCTGAACGGCGAGAGGGTGAGCTTCCCCGTGTGCA
CCTACGTGCCCGCCACCCTGTGCGACCAGATGACCGGCATCCTGGCCACCGACGTGAGCGC
CGACGACGCCCAGAAGCTGCTGGTGGGCCTGAACCAGAGGATCGTGGTGAACGGCAGGACC
CAGAGGAACACCAACACCATGAAGAACTACCTGCTGCCCGTGGTGGCCCAGGCCTTCGCCA
GGTGGGCCAAGGAGTACAAGGAGGACCAGGAGGACGAGAGGCCCCTGGGCCTGAGGGACC
GACAGCTGGTGATGGGCTGCTGCTGGGCCTTCAGGCGGCACAAGATCACCAGCATCTACAA
GAGGCCCGACACCCAGACCATCATCAAGGTGAACAGCGACTTCCACAGCTTCGTGCTGCCC
AGGATCGGCAGCAACACCCTGGAGATCGGCCTGAGGACCCGGATCAGGAAGATGCTGGAG
GAGCACAAGGAGCCCAGCCCTCTGATCACCGCCGAGGACGTGCAGGAGGCCAAGTGCGCC
GCCGACGAGGCCAAGGAGGTGAGGGAGGCCGAGGAGCTGAGGGCCGCCCTGCCTCCCCTG
GCCGCCGACGTGGAGGAGCCCACCCTGGAGGCCGACGTGGACCTGATGCTGCAGGAGGCCG
GCGCCGGCAGCGTGGAGACACCCAGGGGCCTGATCAAGGTGACCAGCTACGACGGCGAGG
ACAAGATCGGCAGCTACGCCGTGCTCAGCCCTCAGGCCGTGCTGAAGTCCGAGAAGCTGAG
CTGCATCCACCCTCTGGCCGAGCAGGTGATCGTGATCACCCACAGCGGCAGGAAGGGCAGG
TACGCCGTGGAGCCCTACCACGGCAAGGTGGTGGTCCCCGAGGGCCACGCCATCCCCGTGC
AGGACTTCCAGGCCCTGAGCGAGAGCGCCACCATCGTGTATAACGAGAGGGAGTTCGTGAA
CAGGTACCTGCACCACATCGCCACCCACGGCGGCGCCCTGAACACCGACGAGGAGTACTAC
AAGACCGTGAAGCCCAGCGAGCACGACGGCGAGTACCTGTACGACATCGACAGGAAGCAG
TGCGTGAAGAAGGAGCTGGTGACCGGCCTGGGCCTGACCGGCGAGCTGGTGGACCCTCCCT
TCCACGAGTTCGCCTACGAGAGCCTGAGGACCAGGCCCGCCGCTCCCTACCAGGTGCCCAC
CATCGGCGTGTACGGCGTGCCCGGCAGCGGCAAGAGCGGCATCATCAAGAGCGCCGTGACC
AAGAAGGACCTGGTGGTGAGCGCCAAGAAGGAGAACTGCGCCGAGATCATCAGGGACGTG
AAGAAGATGAAGGGCCTGGACGTGAACGCCAGGACCGTGGACAGCGTGCTCCTGAACGGCT
GCAAGCACCCCGTGGAGACACTGTATATCGACGAGGCCTTCGCCTGCCACGCCGGCACCCT
GAGGGCCCTGATCGCCATCATCAGGCCCAAGAAGGCCGTGCTGTGCGGCGACCCCAAGCAG
TGCGGCTTCTTCAACATGATGTGCCTGAAGGTGCACTTCAACCACGAGATCTGCACCCAGGT
GTTCCACAAGAGCATCAGCAGGCGGTGCACCAAGAGCGTGACCAGCGTGGTGAGCACCCTG
TTCTACGACAAGAAGATGAGGACCACCAACCCCAAGGAGACAAAGATCGTGATCGACACCA
CCGGCAGCACCAAGCCCAAGCAGGACGACCTGATCCTGACCTGCTTCAGGGGCTGGGTGAA
GCAGCTGCAGATCGACTACAAGGGCAACGAGATCATGACCGCCGCCGCTAGCCAGGGCCTG
ACCAGGAAGGGCGTGTACGCCGTGAGGTACAAGGTGAACGAGAATCCCCTGTACGCCCCTA
CCAGCGAGCACGTGAACGTCCTGCTGACCAGGACCGAGGACAGGATCGTGTGGAAGACCCT
GGCCGGCGACCCCTGGATCAAGACCCTGACCGCCAAGTACCCCGGCAACTTCACCGCCACC
ATCGAGGAGTGGCAGGCCGAGCACGACGCCATCATGAGGCACATCCTGGAGAGGCCCGACC
CCACCGACGTGTTCCAGAACAAGGCCAACGTGTGCTGGGCCAAGGCCCTGGTGCCCGTGCT
GAAGACCGCCGGCATCGACATGACCACCGAGCAGTGGAACACCGTGGACTACTTCGAGACA
GACAAGGCCCACAGCGCCGAGATCGTGCTGAACCAGCTGTGCGTGAGGTTCTTCGGCCTGG
ACCTGGACAGCGGCCTGTTCAGCGCCCCTACCGTGCCCCTGAGCATCAGGAACAACCACTG
GGACAACAGCCCCAGCCCCAACATGTACGGCCTGAACAAGGAGGTGGTGAGGCAGCTGAG
CAGGCGGTACCCTCAGCTGCCCAGGGCCGTGGCCACCGGCAGGGTGTACGACATGAACACC
GGCACCCTGAGGAACTACGACCCCAGGATCAACCTGGTGCCCGTGAACAGGCGGCTGCCAC
ACGCCCTGGTGCTGCACCACAACGAGCACCCTCAGAGCGACTTCAGCAGCTTCGTGAGCAA
GCTGAAGGGCAGGACCGTGCTGGTGGTGGGCGAGAAGCTGAGCGTGCCCGGCAAGATGGT
GGACTGGCTGAGCGACAGGCCCGAGGCCACCTTCCGGGCCAGGCTGGACCTGGGCATCCCC
GGCGACGTGCCCAAGTACGACATCATCTTCGTGAACGTGAGGACCCCTTACAAGTACCACC
ACTACCAGCAGTGCGAGGACCACGCCATCAAGCTGAGCATGCTGACCAAGAAGGCCTGCCT
GCACCTGAACCCCGGCGGCACCTGCGTGAGCATCGGCTACGGCTACGCCGACAGGGCCAGC
GAGAGCATCATCGGCGCCATCGCCAGGCTGTTCAAGTTCAGCAGGGTGTGCAAGCCCAAGA
GCAGCCTGGAGGAGACAGAGGTGCTGTTCGTGTTCATCGGCTACGACCGGAAGGCCAGGAC
CCACAACCCCTACAAGCTGAGCAGCACCCTGACCAACATCTACACCGGCAGCAGGCTGCAC
GAGGCCGGCTGCGCCCCTAGCTACCACGTGGTGAGGGGCGACATCGCCACCGCCACCGAGG
GCGTGATCATCAACGCCGCCAACAGCAAGGGCCAGCCCGGCGGCGGGGTGTGCGGCGCCCT
GTATAAGAAGTTCCCCGAGAGCTTCGACCTGCAGCCCATCGAGGTGGGCAAGGCCAGGCTG
GTGAAGGGCGCCGCCAAGCACATCATCCACGCCGTGGGCCCCAACTTCAACAAGGTGAGCG
AGGTGGAGGGCGACAAGCAGCTGGCCGAGGCCTACGAGAGCATCGCCAAGATCGTGAACG
ACAACAACTACAAGAGCGTGGCCATCCCTCTGCTGAGCACCGGCATCTTCAGCGGCAACAA
GGACAGGCTGACCCAGAGCCTGAACCACCTGCTGACCGCCCTGGACACCACCGACGCCGAC
GTGGCCATCTACTGCAGGGACAAGAAGTGGGAGATGACCCTGAAGGAGGCCGTGGCCAGG
CGGGAGGCCGTGGAGGAGATCTGCATCAGCGACGACAGCAGCGTGACGGAGCCCGACGCC
GAGCTGGTGAGGGTGCACCCCAAGAGCAGCCTGGCCGGCAGGAAGGGCTACAGCACCAGC
GACGGCAAGACCTTCAGCTACCTGGAGGGCACCAAGTTCCACCAGGCCGCCAAGGACATCG
CCGAGATCAACGCCATGTGGCCCGTGGCCACCGAGGCCAACGAGCAGGTGTGCATGTATAT
CCTGGGCGAGAGCATGAGCAGCATCAGGAGCAAGTGCCCCGTGGAGGAGAGCGAGGCCAG
CACCCCTCCCAGCACCCTGCCCTGCCTGTGCATCCACGCCATGACCCCTGAGAGGGTGCAGC
GGCTGAAGGCCAGCAGGCCCGAGCAGATCACCGTGTGCAGCAGCTTCCCTCTGCCCAAGTA
CCGGATCACCGGCGTGCAGAAGATCCAGTGCAGCCAGCCCATCCTGTTCAGCCCCAAGGTG
CCCGCCTACATCCACCCCAGGAAGTACCTGGTGGAGACACCCCCCGTGGACGAGACACCCG
AGCCCAGCGCCGAGAACCAGAGCACCGAGGGCACCCCTGAGCAGCCTCCCCTGATCACCGA
GGACGAGACAAGGACCAGGACGCCCGAGCCCATCATCATTGAGGAGGAAGAGGAGGACAG
CATCAGCCTGCTGAGCGACGGCCCCACCCACCAGGTGCTGCAGGTGGAGGCCGACATCCAC
GGCCCTCCCAGCGTGAGCAGCTCCAGCTGGAGCATCCCTCACGCCAGCGACTTCGACGTGG
ACAGCCTGAGCATCCTGGACACCCTGGAGGGCGCCAGCGTGACCAGCGGCGCCACCAGCGC
CGAGACAAACAGCTACTTCGCCAAGAGCATGGAGTTCCTGGCCAGGCCCGTGCCCGCCCCT
AGGACCGTGTTCAGGAACCCTCCCCACCCCGCCCCTAGGACCAGGACCCCTAGCCTGGCCC
CTAGCAGGGCCTGCAGCAGGACCAGCCTGGTGAGCACCCCTCCCGGCGTGAACCGGGTGAT
CACCAGGGAGGAGCTGGAGGCCCTGACCCCTAGCAGGACCCCTAGCAGGAGCGTGAGCAG
GACCAGCCTGGTGAGCAACCCTCCCGGCGTGAACCGGGTGATCACCAGGGAGGAGTTCGAG
GCCTTCGTGGCCCAGCAGCAAAGGCGGTTCGACGCCGGCGCCTACATCTTCAGCAGCGACA
CCGGCCAGGGCCACCTGCAGCAGAAGTCCGTGAGGCAGACCGTGCTGAGCGAGGTGGTCCT
GGAGAGGACGGAGCTGGAGATCAGCTACGCCCCTAGGCTGGACCAGGAGAAGGAGGAGCT
GCTGAGGAAGAAGCTGCAGCTGAACCCCACCCCTGCCAACAGGAGCAGGTACCAGAGCAG
GAAGGTGGAGAACATGAAGGCCATCACCGCCAGGCGGATCCTGCAGGGCCTGGGCCACTAC
CTGAAGGCCGAGGGCAAGGTGGAGTGCTACAGGACCCTGCACCCCGTGCCCCTGTACTCCA
GCTCCGTGAACAGGGCCTTCAGCAGCCCCAAGGTGGCCGTGGAGGCCTGCAACGCCATGCT
GAAGGAGAACTTCCCCACCGTGGCCAGCTACTGCATCATCCCCGAGTACGACGCCTACCTG
GACATGGTGGACGGCGCCAGCTGCTGCCTGGACACCGCCAGCTTCTGCCCCGCCAAGCTGA
GGAGCTTCCCCAAGAAGCACAGCTACCTGGAGCCCACCATCAGGAGCGCCGTGCCCAGCGC
CATCCAGAACACCCTGCAGAACGTGCTGGCCGCCGCTACCAAGAGGAACTGCAACGTGACC
CAGATGAGGGAGCTGCCCGTGCTGGACAGCGCCGCCTTCAACGTGGAGTGCTTCAAGAAGT
ACGCCTGCAACAACGAGTACTGGGAGACATTCAAGGAGAACCCCATCAGGCTGACCGAGGA
GAACGTGGTGAACTACATCACCAAGCTGAAGGGCCCCAAGGCCGCCGCTCTGTTCGCCAAG
ACCCACAACCTGAACATGCTCCAGGACATCCCTATGGACAGGTTCGTGATGGACCTGAAGA
GGGACGTGAAGGTGACCCCTGGCACCAAGCACACCGAGGAGAGGCCCAAGGTGCAGGTGA
TCCAGGCCGCCGACCCTCTGGCCACCGCCTACCTGTGCGGCATCCACAGGGAGCTGGTGAG
GCGGCTGAACGCCGTCCTGCTGCCCAACATCCACACCCTGTTCGACATGAGCGCCGAGGAC
TTCGACGCCATCATCGCCGAGCACTTCCAGCCCGGCGACTGCGTGCTGGAGACAGACATCG
CCAGCTTCGACAAGAGCGAGGACGACGCTATGGCCCTGACCGCCCTGATGATCCTGGAGGA
CCTGGGCGTGGACGCCGAGCTGCTGACCCTGATCGAGGCCGCCTTCGGCGAGATCAGCAGC
ATCCACCTGCCCACCAAGACCAAGTTCAAGTTCGGCGCCATGATGAAGTCCGGCATGTTCCT
GACCCTGTTCGTGAACACCGTGATCAACATCGTGATCGCCAGCAGGGTGCTGCGGGAGAGG
CTGACCGGCAGCCCCTGCGCCGCCTTCATCGGCGACGACAACATCGTGAAGGGCGTGAAGT
CCGACAAGCTGATGGCCGACAGGTGCGCCACCTGGCTGAACATGGAGGTGAAGATCATCGA
CGCCGTGGTGGGCGAGAAGGCCCCTTACTTCTGCGGCGGCTTCATCCTGTGCGACAGCGTGA
CCGGCACCGCCTGCAGGGTGGCCGACCCTCTGAAGAGGCTGTTCAAGCTGGGCAAGCCCCT
GGCCGCCGACGACGAGCACGACGACGATAGGCGGAGGGCCCTGCACGAGGAGAGCACCAG
GTGGAACCGGGTGGGCATCCTGAGCGAGCTGTGCAAGGCCGTGGAGAGCAGGTACGAGAC
AGTGGGCACCAGCATCATCGTGATGGCCATGACCACCCTGGCCAGCAGCGTCAAGTCCTTC
AGCTACCTGAGGGGGGCCCCTATAACTCTCTACGGCTAACCTGAATGGACTACGACATAGTC
TAGTCCGCCAAGGCCCTTAAGCTCCCTCCCCCCCCCCTAACGTTACTGGCCGAAGCCGCTTG
GAATAAGGCCGGTGTGCGTTTGTCTATATGTTATTTTCCACCATATTGCCGTCTTTTGGCAAT
GTGAGGGCCCGGAAACCTGGCCCTGTCTTCTTGACGAGCATTCCTAGGGGTCTTTCCCCTCT
CGCCAAAGGAATGCAAGGTCTGTTGAATGTCGTGAAGGAAGCAGTTCCTCTGGAAGCTTCTT
GAAGACAAACAACGTCTGTAGCGACCCTTTGCAGGCAGCGGAACCCCCCACCTGGCGACAG
GTGCCTCTGCGGCCAAAAGCCACGTGTATAAGATACACCTGCAAAGGCGGCACAACCCCAG
TGCCACGTTGTGAGTTGGATAGTTGTGGAAAGAGTCAAATGGCTCTCCTCAAGCGTATTCAA
CAAGGGGCTGAAGGATGCCCAGAAGGTACCCCATTGTATGGGATCTGATCTGGGGCCTCGG
TGCACATGCTTTACGTGTGTTTAGTCGAGGTTAAAAAACGTCTAGGCCCCCCGAACCACGGG
GACGTGGTTTTCCTTTGAAAAACACGATGATAATATGAATCCTAACCAGAAGATCATCACCA
TCGGCAGCGTGAGCCTGACCATCAGCACCATCTGCTTCTTCATGCAGATCGCCATCCTGATC
ACCACCGTGACCCTGCACTTCAAGCAGTACGAGTTCAACAGCCCTCCCAACAACCAGGTGA
TGCTGTGCGAGCCCACCATCATCGAGAGGAACATGACCGAGATCGTGTACCTGACCAACAC
CACCATCGAGAAGGAGATCTGCCCCAAGCCCGCCGAGTACAGGAACTGGAGCAAGCCCCA
GTGCGGCATCACCGGCTTCGCCCCTTTCAGCAAGGACAACAGCATCAGGCTGAGCGCCGGC
GGCGACATCTGGGTGACCAGGGAGCCCTACGTGAGCTGCGACCTGGACAAGTGCTACCAGT
TCGCCCTGGGCCAGGGCACCACCCTGAACAACGTGCACAGCAACAACACCGTGAGGGACAG
GACCCCTTACCGGACCCTGCTGATGAACGAGCTGGGCGTGCCCTTCCACCTGGGCACCAAG
CAGGTGTGCATCGCCTGGAGCAGCTCCAGCTGCCACGACGGCAAGGCCTGGCTGCACGTGT
GCATCACCGGCGACGACAAGAACGCCACCGCCAGCTTCATCTACAACGGCAGGCTGGTGGA
CAGCGTGGTGAGCTGGAGCAACGACATCCTGAGGACCCAGGAGAGCGAGTGCGTGTGCATC
AACGGCACCTGCACCGTGGTGATGACCGACGGCAACGCCACCGGCAAGGCCGACACCAAG
ATCCTGTTCATCGAGGAGGGCAAGATCGTGCACACCAGCAAGCTGAGCGGCAGCGCCCAGC
ACGTGGAGGAGTGCAGCTGCTACCCCAGGTACCCCGGCGTGAGGTGCGTGTGCAGGGACAA
CTGGAAGGGCAGCAACCGGCCCATCATCGACATCAACATCAAGGACCACAGCATCGTCAGC
AGGTACGTGTGCAGCGGCCTGGTGGGCGACACCCCTAGAAAGAGCGACAGCAGCTCCAGCA
GCCACTGCCTGAACCCCAACAACGAGAAGGGCGACCACGGCGTGAAGGGCTGGGCCTTCGA
CGACGGCAACGACGTGTGGATGGGCAGGACCATCAACGAGACAAGCAGGCTGGGCTACGA
GACATTCAAGGTGGTGGAGGGCTGGAGCAACCCCAAGAGCAAGCTGCAGATCAACAGGCA
GGTGATCGTGGACAGGGGCGACAGGAGCGGCTACAGCGGCATCTTCAGCGTGGAGGGCAA
GAGCTGCATCAACCGGTGCTTCTACGTGGAGCTGATCAGGGGCAGGAAGGAGGAGACAGA
GGTGCTGTGGACCAGCAACAGCATCGTGGTGTTCTGCGGCACCAGCGGCACCTACGGCACC
GGCAGCTGGCCCGACGGCGCCAACCTGAGCCTGATGCACATCTGATAGTAAACTCGACCGG
AAACGCAATAGCCGAAAAACAAAAAACAAAAAAAACAAAAAAAAAACCAAAAAAACAAA
ACACATTAAAACAGCCTGTGGGTTGATCCCACCCACAGGCCCATTGGGCGCTAGCACTCTGG
TATCACGGTACCTTTGTGCGCCTGTTTTATACCCCCTCCCCCAACTGTAACTTAGAAGTAACA
CACACCGATCAACAGTCAGCGTGGCACACCAGCCACGTTTTGATCAAGCACTTCTGTTACCC
CGGACTGAGTATCAATAGACTGCTCACGCGGTTGAAGGAGAAAGCGTTCGTTATCCGGCCA
ACTACTTCGAAAAACCTAGTAACACCGTGGAAGTTGCAGAGTGTTTCGCTCAGCACTACCCC
AGTGTAGATCAGGTCGATGAGTCACCGCATTCCCCACGGGCGACCGTGGCGGTGGCTGCGT
TGGCGGCCTGCCCATGGGGAAACCCATGGGACGCTCTAATACAGACATGGTGCGAAGAGTC
TATTGAGCTAGTTGGTAGTCCTCCGGCCCCTGAATGCGGCTAATCCTAACTGCGGAGCACAC
ACCCTCAAGCCAGAGGGCAGTGTGTCGTAACGGGCAACTCTGCAGCGGAACCGACTACTTT
GGGTGTCCGTGTTTCATTTTATTCCTATACTGGCTGCTTATGGTGACAATTGAGAGATCGTTA
CCATATAGCTATTGGATTGGCCATCCGGTGACTAATAGAGCTATTATATATCCCTTTGTTGG
GTTTATACCACTTAGCTTGAAAGAGGTTAAAACATTACAATTCATTGTTAAGTTGAATACAG
CAAAATGAAGACCATCATCGCCCTGAGCTACATCCTGTGCCTGGTGTTCGCCCAGAAGATCC
CCGGCAATGACAACAGCACCGCCACCCTGTGCCTGGGCCACCACGCCGTGCCCAACGGCAC
CATCGTGAAGACCATCACCAACGACAGGATCGAGGTGACCAACGCCACCGAGCTGGTGCAG
AACAGCAGCATCGGCGAGATCTGCGACAGCCCTCACCAGATCCTGGACGGCGGCAACTGCA
CCCTGATCGACGCCCTGCTGGGCGACCCTCAGTGCGACGGCTTCCAGAACAAGGAGTGGGA
CCTGTTCGTGGAGAGGAGCAGGGCCAACAGCAACTGCTACCCCTACGACGTGCCCGACTAC
GCCAGCCTGAGGAGCCTGGTGGCCAGCAGCGGCACCCTGGAGTTCAAGAACGAGAGCTTCA
ACTGGACCGGCGTGAAGCAGAACGGCACCAGCAGCGCCTGCATCAGGGGCAGCAGCTCCA
GCTTCTTCAGCCGGCTGAATTGGCTCACCCACCTGAACTACACCTACCCCGCCCTGAACGTG
ACCATGCCCAACAACGAGCAGTTCGACAAGCTGTATATCTGGGGCGTGCACCACCCCAGCA
CCGACAAGGACCAGATCAGCCTGTTCGCCCAGCCCAGCGGCAGGATCACCGTGAGCACCAA
GAGGAGCCAGCAGGCCGTGATCCCCAACATCGGCAGCAGGCCCAGGATCAGGGACATCCCC
AGCAGGATCAGCATCTACTGGACCATCGTGAAGCCCGGCGACATCCTGCTGATCAACAGCA
CCGGCAACCTGATCGCCCCTAGGGGCTACTTCAAGATCAGGAGCGGCAAGAGCAGCATCAT
GAGGAGCGACGCCCCTATCGGCAAGTGCAAGAGCGAGTGCATCACCCCTAACGGCAGCATC
CCCAACGACAAGCCCTTCCAGAACGTGAACAGGATCACCTACGGCGCCTGCCCCAGGTACG
TGAAGCAGAGCACCCTGAAGCTGGCCACCGGCATGAGGAACGTGCCCGAGAAGCAGACCA
GGGGCATCTTCGGCGCCATCGCCGGCTTCATCGAGAACGGCTGGGAGGGCATGGTGGACGG
CTGGTACGGCTTCAGGCACCAGAACAGCGAGGGCAGGGGCCAGGCCGCCGACCTGAAGTCC
ACCCAGGCCGCCATCGACCAGATCAACGGCAAGCTGAACAGGCTGATCGGCAAGACCAAC
GAGAAGTTCCACCAGATCGAGAAGGAGTTCAGCGAGGTGGAGGGCAGGGTGCAGGACCTG
GAGAAGTACGTGGAGGACACCAAGATCGACCTGTGGAGCTACAACGCCGAGCTGCTGGTGG
CCCTGGAGAACCAGCACACCATCGACCTGACCGACAGCGAGATGAACAAGCTGTTCGAGAA
GACCAAGAAGCAGCTGAGGGAGAACGCCGAGGACATGGGCAACGGCTGCTTCAAGATCTA
CCACAAGTGCGACAACGCCTGCATCGGCAGCATCAGGAACGAGACATATGACCACAACGTG
TACCGGGACGAGGCCCTGAACAACCGGTTCCAGATCAAGGGCGTGGAGCTGAAGTCCGGCT
ACAAGGACTGGATCCTGTGGATCAGCTTCGCCATGAGCTGCTTCCTGCTGTGCATCGCCCTG
CTGGGCTTCATCATGTGGGCCTGCCAGAAGGGCAACATCAGGTGCAACATCTGCATCTGATA
GTAAACTCGAGTATGTTACGTGCAAAGGTGATTGTCACCCCCCGAAAGACCATATTGTGACA
CACCCTCAGTATCACGCCCAAACATTTACAGCCGCGGTGTCAAAAACCGCGTGGACGTGGTT
AACATCCCTGCTGGGAGGATCAGCCGTAATTATTATAATTGGCTTGGTGCTGGCTACTATTG
TGGCCATGTACGTGCTGACCAACCAGAAACATAATTGAATACAGCAGCAATTGGCAAGCTG
CTTACATAGAACTCGCGGCGATTGGCATGCCGCCTTAAAATTTTTATTTTATTTTTTCTTTTCT
TTTCCGAATCGGATTTTGTTTTTAATATTTC
SEQ ID NO: 153 (sgP CVB3 NA sgP EMCV HA)
ATGGGCGGCGCATGAGAGAAGCCCAGACCAATTACCTACCCAAAATGGAGAAAGTTCACGT
TGACATCGAGGAAGACAGCCCATTCCTCAGAGCTTTGCAGCGGAGCTTCCCGCAGTTTGAG
GTAGAAGCCAAGCAGGTCACTGATAATGACCATGCTAATGCCAGAGCGTTTTCGCATCTGG
CTTCAAAACTGATCGAAACGGAGGTGGACCCATCCGACACGATCCTTGACATTGGAAGTGC
GCCCGCCCGCAGAATGTATTCTAAGCACAAGTATCATTGTATCTGTCCGATGAGATGTGCGG
AAGATCCGGACAGATTGTATAAGTATGCAACTAAGCTGAAGAAAAACTGTAAGGAAATAAC
TGATAAGGAATTGGACAAGAAAATGAAGGAGCTGGCCGCCGTCATGAGCGACCCTGACCTG
GAAACTGAGACTATGTGCCTCCACGACGACGAGTCGTGTCGCTACGAAGGGCAAGTCGCTG
TTTACCAGGATGTATACGCCGTCGACGGCCCCACCAGCCTGTACCACCAGGCCAACAAGGG
CGTGAGGGTGGCCTACTGGATCGGCTTCGACACCACACCCTTCATGTTCAAGAACCTGGCCG
GCGCCTACCCCAGCTACAGCACCAACTGGGCCGACGAGACAGTGCTGACCGCCAGGAACAT
CGGCCTGTGCAGCAGCGACGTGATGGAGAGGAGCCGGAGGGGCATGAGCATCCTGAGGAA
GAAGTACCTGAAGCCCAGCAACAACGTGCTGTTCAGCGTGGGCAGCACCATCTACCACGAG
AAGAGGGACCTGCTGAGGAGCTGGCACCTGCCCAGCGTGTTCCACCTGAGGGGCAAGCAGA
ACTACACCTGCAGGTGCGAGACAATCGTGAGCTGCGACGGCTACGTGGTGAAGAGGATCGC
CATCAGCCCCGGCCTGTACGGCAAGCCCAGCGGCTACGCCGCCACCATGCACAGGGAGGGC
TTCCTGTGCTGCAAGGTGACCGACACCCTGAACGGCGAGAGGGTGAGCTTCCCCGTGTGCA
CCTACGTGCCCGCCACCCTGTGCGACCAGATGACCGGCATCCTGGCCACCGACGTGAGCGC
CGACGACGCCCAGAAGCTGCTGGTGGGCCTGAACCAGAGGATCGTGGTGAACGGCAGGACC
CAGAGGAACACCAACACCATGAAGAACTACCTGCTGCCCGTGGTGGCCCAGGCCTTCGCCA
GGTGGGCCAAGGAGTACAAGGAGGACCAGGAGGACGAGAGGCCCCTGGGCCTGAGGGACC
GACAGCTGGTGATGGGCTGCTGCTGGGCCTTCAGGCGGCACAAGATCACCAGCATCTACAA
GAGGCCCGACACCCAGACCATCATCAAGGTGAACAGCGACTTCCACAGCTTCGTGCTGCCC
AGGATCGGCAGCAACACCCTGGAGATCGGCCTGAGGACCCGGATCAGGAAGATGCTGGAG
GAGCACAAGGAGCCCAGCCCTCTGATCACCGCCGAGGACGTGCAGGAGGCCAAGTGCGCC
GCCGACGAGGCCAAGGAGGTGAGGGAGGCCGAGGAGCTGAGGGCCGCCCTGCCTCCCCTG
GCCGCCGACGTGGAGGAGCCCACCCTGGAGGCCGACGTGGACCTGATGCTGCAGGAGGCCG
GCGCCGGCAGCGTGGAGACACCCAGGGGCCTGATCAAGGTGACCAGCTACGACGGCGAGG
ACAAGATCGGCAGCTACGCCGTGCTCAGCCCTCAGGCCGTGCTGAAGTCCGAGAAGCTGAG
CTGCATCCACCCTCTGGCCGAGCAGGTGATCGTGATCACCCACAGCGGCAGGAAGGGCAGG
TACGCCGTGGAGCCCTACCACGGCAAGGTGGTGGTCCCCGAGGGCCACGCCATCCCCGTGC
AGGACTTCCAGGCCCTGAGCGAGAGCGCCACCATCGTGTATAACGAGAGGGAGTTCGTGAA
CAGGTACCTGCACCACATCGCCACCCACGGCGGCGCCCTGAACACCGACGAGGAGTACTAC
AAGACCGTGAAGCCCAGCGAGCACGACGGCGAGTACCTGTACGACATCGACAGGAAGCAG
TGCGTGAAGAAGGAGCTGGTGACCGGCCTGGGCCTGACCGGCGAGCTGGTGGACCCTCCCT
TCCACGAGTTCGCCTACGAGAGCCTGAGGACCAGGCCCGCCGCTCCCTACCAGGTGCCCAC
CATCGGCGTGTACGGCGTGCCCGGCAGCGGCAAGAGCGGCATCATCAAGAGCGCCGTGACC
AAGAAGGACCTGGTGGTGAGCGCCAAGAAGGAGAACTGCGCCGAGATCATCAGGGACGTG
AAGAAGATGAAGGGCCTGGACGTGAACGCCAGGACCGTGGACAGCGTGCTCCTGAACGGCT
GCAAGCACCCCGTGGAGACACTGTATATCGACGAGGCCTTCGCCTGCCACGCCGGCACCCT
GAGGGCCCTGATCGCCATCATCAGGCCCAAGAAGGCCGTGCTGTGCGGCGACCCCAAGCAG
TGCGGCTTCTTCAACATGATGTGCCTGAAGGTGCACTTCAACCACGAGATCTGCACCCAGGT
GTTCCACAAGAGCATCAGCAGGCGGTGCACCAAGAGCGTGACCAGCGTGGTGAGCACCCTG
TTCTACGACAAGAAGATGAGGACCACCAACCCCAAGGAGACAAAGATCGTGATCGACACCA
CCGGCAGCACCAAGCCCAAGCAGGACGACCTGATCCTGACCTGCTTCAGGGGCTGGGTGAA
GCAGCTGCAGATCGACTACAAGGGCAACGAGATCATGACCGCCGCCGCTAGCCAGGGCCTG
ACCAGGAAGGGCGTGTACGCCGTGAGGTACAAGGTGAACGAGAATCCCCTGTACGCCCCTA
CCAGCGAGCACGTGAACGTCCTGCTGACCAGGACCGAGGACAGGATCGTGTGGAAGACCCT
GGCCGGCGACCCCTGGATCAAGACCCTGACCGCCAAGTACCCCGGCAACTTCACCGCCACC
ATCGAGGAGTGGCAGGCCGAGCACGACGCCATCATGAGGCACATCCTGGAGAGGCCCGACC
CCACCGACGTGTTCCAGAACAAGGCCAACGTGTGCTGGGCCAAGGCCCTGGTGCCCGTGCT
GAAGACCGCCGGCATCGACATGACCACCGAGCAGTGGAACACCGTGGACTACTTCGAGACA
GACAAGGCCCACAGCGCCGAGATCGTGCTGAACCAGCTGTGCGTGAGGTTCTTCGGCCTGG
ACCTGGACAGCGGCCTGTTCAGCGCCCCTACCGTGCCCCTGAGCATCAGGAACAACCACTG
GGACAACAGCCCCAGCCCCAACATGTACGGCCTGAACAAGGAGGTGGTGAGGCAGCTGAG
CAGGCGGTACCCTCAGCTGCCCAGGGCCGTGGCCACCGGCAGGGTGTACGACATGAACACC
GGCACCCTGAGGAACTACGACCCCAGGATCAACCTGGTGCCCGTGAACAGGCGGCTGCCAC
ACGCCCTGGTGCTGCACCACAACGAGCACCCTCAGAGCGACTTCAGCAGCTTCGTGAGCAA
GCTGAAGGGCAGGACCGTGCTGGTGGTGGGCGAGAAGCTGAGCGTGCCCGGCAAGATGGT
GGACTGGCTGAGCGACAGGCCCGAGGCCACCTTCCGGGCCAGGCTGGACCTGGGCATCCCC
GGCGACGTGCCCAAGTACGACATCATCTTCGTGAACGTGAGGACCCCTTACAAGTACCACC
ACTACCAGCAGTGCGAGGACCACGCCATCAAGCTGAGCATGCTGACCAAGAAGGCCTGCCT
GCACCTGAACCCCGGCGGCACCTGCGTGAGCATCGGCTACGGCTACGCCGACAGGGCCAGC
GAGAGCATCATCGGCGCCATCGCCAGGCTGTTCAAGTTCAGCAGGGTGTGCAAGCCCAAGA
GCAGCCTGGAGGAGACAGAGGTGCTGTTCGTGTTCATCGGCTACGACCGGAAGGCCAGGAC
CCACAACCCCTACAAGCTGAGCAGCACCCTGACCAACATCTACACCGGCAGCAGGCTGCAC
GAGGCCGGCTGCGCCCCTAGCTACCACGTGGTGAGGGGCGACATCGCCACCGCCACCGAGG
GCGTGATCATCAACGCCGCCAACAGCAAGGGCCAGCCCGGCGGCGGGGTGTGCGGCGCCCT
GTATAAGAAGTTCCCCGAGAGCTTCGACCTGCAGCCCATCGAGGTGGGCAAGGCCAGGCTG
GTGAAGGGCGCCGCCAAGCACATCATCCACGCCGTGGGCCCCAACTTCAACAAGGTGAGCG
AGGTGGAGGGCGACAAGCAGCTGGCCGAGGCCTACGAGAGCATCGCCAAGATCGTGAACG
ACAACAACTACAAGAGCGTGGCCATCCCTCTGCTGAGCACCGGCATCTTCAGCGGCAACAA
GGACAGGCTGACCCAGAGCCTGAACCACCTGCTGACCGCCCTGGACACCACCGACGCCGAC
GTGGCCATCTACTGCAGGGACAAGAAGTGGGAGATGACCCTGAAGGAGGCCGTGGCCAGG
CGGGAGGCCGTGGAGGAGATCTGCATCAGCGACGACAGCAGCGTGACGGAGCCCGACGCC
GAGCTGGTGAGGGTGCACCCCAAGAGCAGCCTGGCCGGCAGGAAGGGCTACAGCACCAGC
GACGGCAAGACCTTCAGCTACCTGGAGGGCACCAAGTTCCACCAGGCCGCCAAGGACATCG
CCGAGATCAACGCCATGTGGCCCGTGGCCACCGAGGCCAACGAGCAGGTGTGCATGTATAT
CCTGGGCGAGAGCATGAGCAGCATCAGGAGCAAGTGCCCCGTGGAGGAGAGCGAGGCCAG
CACCCCTCCCAGCACCCTGCCCTGCCTGTGCATCCACGCCATGACCCCTGAGAGGGTGCAGC
GGCTGAAGGCCAGCAGGCCCGAGCAGATCACCGTGTGCAGCAGCTTCCCTCTGCCCAAGTA
CCGGATCACCGGCGTGCAGAAGATCCAGTGCAGCCAGCCCATCCTGTTCAGCCCCAAGGTG
CCCGCCTACATCCACCCCAGGAAGTACCTGGTGGAGACACCCCCCGTGGACGAGACACCCG
AGCCCAGCGCCGAGAACCAGAGCACCGAGGGCACCCCTGAGCAGCCTCCCCTGATCACCGA
GGACGAGACAAGGACCAGGACGCCCGAGCCCATCATCATTGAGGAGGAAGAGGAGGACAG
CATCAGCCTGCTGAGCGACGGCCCCACCCACCAGGTGCTGCAGGTGGAGGCCGACATCCAC
GGCCCTCCCAGCGTGAGCAGCTCCAGCTGGAGCATCCCTCACGCCAGCGACTTCGACGTGG
ACAGCCTGAGCATCCTGGACACCCTGGAGGGCGCCAGCGTGACCAGCGGCGCCACCAGCGC
CGAGACAAACAGCTACTTCGCCAAGAGCATGGAGTTCCTGGCCAGGCCCGTGCCCGCCCCT
AGGACCGTGTTCAGGAACCCTCCCCACCCCGCCCCTAGGACCAGGACCCCTAGCCTGGCCC
CTAGCAGGGCCTGCAGCAGGACCAGCCTGGTGAGCACCCCTCCCGGCGTGAACCGGGTGAT
CACCAGGGAGGAGCTGGAGGCCCTGACCCCTAGCAGGACCCCTAGCAGGAGCGTGAGCAG
GACCAGCCTGGTGAGCAACCCTCCCGGCGTGAACCGGGTGATCACCAGGGAGGAGTTCGAG
GCCTTCGTGGCCCAGCAGCAAAGGCGGTTCGACGCCGGCGCCTACATCTTCAGCAGCGACA
CCGGCCAGGGCCACCTGCAGCAGAAGTCCGTGAGGCAGACCGTGCTGAGCGAGGTGGTCCT
GGAGAGGACGGAGCTGGAGATCAGCTACGCCCCTAGGCTGGACCAGGAGAAGGAGGAGCT
GCTGAGGAAGAAGCTGCAGCTGAACCCCACCCCTGCCAACAGGAGCAGGTACCAGAGCAG
GAAGGTGGAGAACATGAAGGCCATCACCGCCAGGCGGATCCTGCAGGGCCTGGGCCACTAC
CTGAAGGCCGAGGGCAAGGTGGAGTGCTACAGGACCCTGCACCCCGTGCCCCTGTACTCCA
GCTCCGTGAACAGGGCCTTCAGCAGCCCCAAGGTGGCCGTGGAGGCCTGCAACGCCATGCT
GAAGGAGAACTTCCCCACCGTGGCCAGCTACTGCATCATCCCCGAGTACGACGCCTACCTG
GACATGGTGGACGGCGCCAGCTGCTGCCTGGACACCGCCAGCTTCTGCCCCGCCAAGCTGA
GGAGCTTCCCCAAGAAGCACAGCTACCTGGAGCCCACCATCAGGAGCGCCGTGCCCAGCGC
CATCCAGAACACCCTGCAGAACGTGCTGGCCGCCGCTACCAAGAGGAACTGCAACGTGACC
CAGATGAGGGAGCTGCCCGTGCTGGACAGCGCCGCCTTCAACGTGGAGTGCTTCAAGAAGT
ACGCCTGCAACAACGAGTACTGGGAGACATTCAAGGAGAACCCCATCAGGCTGACCGAGGA
GAACGTGGTGAACTACATCACCAAGCTGAAGGGCCCCAAGGCCGCCGCTCTGTTCGCCAAG
ACCCACAACCTGAACATGCTCCAGGACATCCCTATGGACAGGTTCGTGATGGACCTGAAGA
GGGACGTGAAGGTGACCCCTGGCACCAAGCACACCGAGGAGAGGCCCAAGGTGCAGGTGA
TCCAGGCCGCCGACCCTCTGGCCACCGCCTACCTGTGCGGCATCCACAGGGAGCTGGTGAG
GCGGCTGAACGCCGTCCTGCTGCCCAACATCCACACCCTGTTCGACATGAGCGCCGAGGAC
TTCGACGCCATCATCGCCGAGCACTTCCAGCCCGGCGACTGCGTGCTGGAGACAGACATCG
CCAGCTTCGACAAGAGCGAGGACGACGCTATGGCCCTGACCGCCCTGATGATCCTGGAGGA
CCTGGGCGTGGACGCCGAGCTGCTGACCCTGATCGAGGCCGCCTTCGGCGAGATCAGCAGC
ATCCACCTGCCCACCAAGACCAAGTTCAAGTTCGGCGCCATGATGAAGTCCGGCATGTTCCT
GACCCTGTTCGTGAACACCGTGATCAACATCGTGATCGCCAGCAGGGTGCTGCGGGAGAGG
CTGACCGGCAGCCCCTGCGCCGCCTTCATCGGCGACGACAACATCGTGAAGGGCGTGAAGT
CCGACAAGCTGATGGCCGACAGGTGCGCCACCTGGCTGAACATGGAGGTGAAGATCATCGA
CGCCGTGGTGGGCGAGAAGGCCCCTTACTTCTGCGGCGGCTTCATCCTGTGCGACAGCGTGA
CCGGCACCGCCTGCAGGGTGGCCGACCCTCTGAAGAGGCTGTTCAAGCTGGGCAAGCCCCT
GGCCGCCGACGACGAGCACGACGACGATAGGCGGAGGGCCCTGCACGAGGAGAGCACCAG
GTGGAACCGGGTGGGCATCCTGAGCGAGCTGTGCAAGGCCGTGGAGAGCAGGTACGAGAC
AGTGGGCACCAGCATCATCGTGATGGCCATGACCACCCTGGCCAGCAGCGTCAAGTCCTTC
AGCTACCTGAGGGGGGCCCCTATAACTCTCTACGGCTAACCTGAATGGACTACGACATAGTC
TAGTCCGCCAAGGCCCTTAAGTTAAAACAGCCTGTGGGTTGATCCCACCCACAGGCCCATTG
GGCGCTAGCACTCTGGTATCACGGTACCTTTGTGCGCCTGTTTTATACCCCCTCCCCCAACTG
TAACTTAGAAGTAACACACACCGATCAACAGTCAGCGTGGCACACCAGCCACGTTTTGATC
AAGCACTTCTGTTACCCCGGACTGAGTATCAATAGACTGCTCACGCGGTTGAAGGAGAAAG
CGTTCGTTATCCGGCCAACTACTTCGAAAAACCTAGTAACACCGTGGAAGTTGCAGAGTGTT
TCGCTCAGCACTACCCCAGTGTAGATCAGGTCGATGAGTCACCGCATTCCCCACGGGCGACC
GTGGCGGTGGCTGCGTTGGCGGCCTGCCCATGGGGAAACCCATGGGACGCTCTAATACAGA
CATGGTGCGAAGAGTCTATTGAGCTAGTTGGTAGTCCTCCGGCCCCTGAATGCGGCTAATCC
TAACTGCGGAGCACACACCCTCAAGCCAGAGGGCAGTGTGTCGTAACGGGCAACTCTGCAG
CGGAACCGACTACTTTGGGTGTCCGTGTTTCATTTTATTCCTATACTGGCTGCTTATGGTGAC
AATTGAGAGATCGTTACCATATAGCTATTGGATTGGCCATCCGGTGACTAATAGAGCTATTA
TATATCCCTTTGTTGGGTTTATACCACTTAGCTTGAAAGAGGTTAAAACATTACAATTCATTG
TTAAGTTGAATACAGCAAAATGAATCCTAACCAGAAGATCATCACCATCGGCAGCGTGAGC
CTGACCATCAGCACCATCTGCTTCTTCATGCAGATCGCCATCCTGATCACCACCGTGACCCT
GCACTTCAAGCAGTACGAGTTCAACAGCCCTCCCAACAACCAGGTGATGCTGTGCGAGCCC
ACCATCATCGAGAGGAACATGACCGAGATCGTGTACCTGACCAACACCACCATCGAGAAGG
AGATCTGCCCCAAGCCCGCCGAGTACAGGAACTGGAGCAAGCCCCAGTGCGGCATCACCGG
CTTCGCCCCTTTCAGCAAGGACAACAGCATCAGGCTGAGCGCCGGCGGCGACATCTGGGTG
ACCAGGGAGCCCTACGTGAGCTGCGACCTGGACAAGTGCTACCAGTTCGCCCTGGGCCAGG
GCACCACCCTGAACAACGTGCACAGCAACAACACCGTGAGGGACAGGACCCCTTACCGGAC
CCTGCTGATGAACGAGCTGGGCGTGCCCTTCCACCTGGGCACCAAGCAGGTGTGCATCGCCT
GGAGCAGCTCCAGCTGCCACGACGGCAAGGCCTGGCTGCACGTGTGCATCACCGGCGACGA
CAAGAACGCCACCGCCAGCTTCATCTACAACGGCAGGCTGGTGGACAGCGTGGTGAGCTGG
AGCAACGACATCCTGAGGACCCAGGAGAGCGAGTGCGTGTGCATCAACGGCACCTGCACCG
TGGTGATGACCGACGGCAACGCCACCGGCAAGGCCGACACCAAGATCCTGTTCATCGAGGA
GGGCAAGATCGTGCACACCAGCAAGCTGAGCGGCAGCGCCCAGCACGTGGAGGAGTGCAG
CTGCTACCCCAGGTACCCCGGCGTGAGGTGCGTGTGCAGGGACAACTGGAAGGGCAGCAAC
CGGCCCATCATCGACATCAACATCAAGGACCACAGCATCGTCAGCAGGTACGTGTGCAGCG
GCCTGGTGGGCGACACCCCTAGAAAGAGCGACAGCAGCTCCAGCAGCCACTGCCTGAACCC
CAACAACGAGAAGGGCGACCACGGCGTGAAGGGCTGGGCCTTCGACGACGGCAACGACGT
GTGGATGGGCAGGACCATCAACGAGACAAGCAGGCTGGGCTACGAGACATTCAAGGTGGT
GGAGGGCTGGAGCAACCCCAAGAGCAAGCTGCAGATCAACAGGCAGGTGATCGTGGACAG
GGGCGACAGGAGCGGCTACAGCGGCATCTTCAGCGTGGAGGGCAAGAGCTGCATCAACCG
GTGCTTCTACGTGGAGCTGATCAGGGGCAGGAAGGAGGAGACAGAGGTGCTGTGGACCAGC
AACAGCATCGTGGTGTTCTGCGGCACCAGCGGCACCTACGGCACCGGCAGCTGGCCCGACG
GCGCCAACCTGAGCCTGATGCACATCTGATAGTAAACTCGAAGCAGTGTTAAATCATTCAGC
TACCTGAGAGGGGCCCCTATAACTCTCTACGGCTAACCTGAATGGACTACGACATAGTCTAG
TCCGCCAAGGCCGAATTCCTCCCTCCCCCCCCCCTAACGTTACTGGCCGAAGCCGCTTGGAA
TAAGGCCGGTGTGCGTTTGTCTATATGTTATTTTCCACCATATTGCCGTCTTTTGGCAATGTG
AGGGCCCGGAAACCTGGCCCTGTCTTCTTGACGAGCATTCCTAGGGGTCTTTCCCCTCTCGC
CAAAGGAATGCAAGGTCTGTTGAATGTCGTGAAGGAAGCAGTTCCTCTGGAAGCTTCTTGA
AGACAAACAACGTCTGTAGCGACCCTTTGCAGGCAGCGGAACCCCCCACCTGGCGACAGGT
GCCTCTGCGGCCAAAAGCCACGTGTATAAGATACACCTGCAAAGGCGGCACAACCCCAGTG
CCACGTTGTGAGTTGGATAGTTGTGGAAAGAGTCAAATGGCTCTCCTCAAGCGTATTCAACA
AGGGGCTGAAGGATGCCCAGAAGGTACCCCATTGTATGGGATCTGATCTGGGGCCTCGGTG
CACATGCTTTACGTGTGTTTAGTCGAGGTTAAAAAACGTCTAGGCCCCCCGAACCACGGGGA
CGTGGTTTTCCTTTGAAAAACACGATGATAATATGAAGACCATCATCGCCCTGAGCTACATC
CTGTGCCTGGTGTTCGCCCAGAAGATCCCCGGCAATGACAACAGCACCGCCACCCTGTGCCT
GGGCCACCACGCCGTGCCCAACGGCACCATCGTGAAGACCATCACCAACGACAGGATCGAG
GTGACCAACGCCACCGAGCTGGTGCAGAACAGCAGCATCGGCGAGATCTGCGACAGCCCTC
ACCAGATCCTGGACGGCGGCAACTGCACCCTGATCGACGCCCTGCTGGGCGACCCTCAGTG
CGACGGCTTCCAGAACAAGGAGTGGGACCTGTTCGTGGAGAGGAGCAGGGCCAACAGCAA
CTGCTACCCCTACGACGTGCCCGACTACGCCAGCCTGAGGAGCCTGGTGGCCAGCAGCGGC
ACCCTGGAGTTCAAGAACGAGAGCTTCAACTGGACCGGCGTGAAGCAGAACGGCACCAGCA
GCGCCTGCATCAGGGGCAGCAGCTCCAGCTTCTTCAGCCGGCTGAATTGGCTCACCCACCTG
AACTACACCTACCCCGCCCTGAACGTGACCATGCCCAACAACGAGCAGTTCGACAAGCTGT
ATATCTGGGGCGTGCACCACCCCAGCACCGACAAGGACCAGATCAGCCTGTTCGCCCAGCC
CAGCGGCAGGATCACCGTGAGCACCAAGAGGAGCCAGCAGGCCGTGATCCCCAACATCGG
CAGCAGGCCCAGGATCAGGGACATCCCCAGCAGGATCAGCATCTACTGGACCATCGTGAAG
CCCGGCGACATCCTGCTGATCAACAGCACCGGCAACCTGATCGCCCCTAGGGGCTACTTCA
AGATCAGGAGCGGCAAGAGCAGCATCATGAGGAGCGACGCCCCTATCGGCAAGTGCAAGA
GCGAGTGCATCACCCCTAACGGCAGCATCCCCAACGACAAGCCCTTCCAGAACGTGAACAG
GATCACCTACGGCGCCTGCCCCAGGTACGTGAAGCAGAGCACCCTGAAGCTGGCCACCGGC
ATGAGGAACGTGCCCGAGAAGCAGACCAGGGGCATCTTCGGCGCCATCGCCGGCTTCATCG
AGAACGGCTGGGAGGGCATGGTGGACGGCTGGTACGGCTTCAGGCACCAGAACAGCGAGG
GCAGGGGCCAGGCCGCCGACCTGAAGTCCACCCAGGCCGCCATCGACCAGATCAACGGCAA
GCTGAACAGGCTGATCGGCAAGACCAACGAGAAGTTCCACCAGATCGAGAAGGAGTTCAGC
GAGGTGGAGGGCAGGGTGCAGGACCTGGAGAAGTACGTGGAGGACACCAAGATCGACCTG
TGGAGCTACAACGCCGAGCTGCTGGTGGCCCTGGAGAACCAGCACACCATCGACCTGACCG
ACAGCGAGATGAACAAGCTGTTCGAGAAGACCAAGAAGCAGCTGAGGGAGAACGCCGAGG
ACATGGGCAACGGCTGCTTCAAGATCTACCACAAGTGCGACAACGCCTGCATCGGCAGCAT
CAGGAACGAGACATATGACCACAACGTGTACCGGGACGAGGCCCTGAACAACCGGTTCCAG
ATCAAGGGCGTGGAGCTGAAGTCCGGCTACAAGGACTGGATCCTGTGGATCAGCTTCGCCA
TGAGCTGCTTCCTGCTGTGCATCGCCCTGCTGGGCTTCATCATGTGGGCCTGCCAGAAGGGC
AACATCAGGTGCAACATCTGCATCTGATAGTAAACTCGAGTATGTTACGTGCAAAGGTGATT
GTCACCCCCCGAAAGACCATATTGTGACACACCCTCAGTATCACGCCCAAACATTTACAGCC
GCGGTGTCAAAAACCGCGTGGACGTGGTTAACATCCCTGCTGGGAGGATCAGCCGTAATTA
TTATAATTGGCTTGGTGCTGGCTACTATTGTGGCCATGTACGTGCTGACCAACCAGAAACAT
AATTGAATACAGCAGCAATTGGCAAGCTGCTTACATAGAACTCGCGGCGATTGGCATGCCG
CCTTAAAATTTTTATTTTATTTTTTCTTTTCTTTTCCGAATCGGATTTTGTTTTTAATATTTC
SEQ ID NO: 154 (sgP EMCV NA sgP EMCV HA)
ATGGGCGGCGCATGAGAGAAGCCCAGACCAATTACCTACCCAAAATGGAGAAAGTTCACGT
TGACATCGAGGAAGACAGCCCATTCCTCAGAGCTTTGCAGCGGAGCTTCCCGCAGTTTGAG
GTAGAAGCCAAGCAGGTCACTGATAATGACCATGCTAATGCCAGAGCGTTTTCGCATCTGG
CTTCAAAACTGATCGAAACGGAGGTGGACCCATCCGACACGATCCTTGACATTGGAAGTGC
GCCCGCCCGCAGAATGTATTCTAAGCACAAGTATCATTGTATCTGTCCGATGAGATGTGCGG
AAGATCCGGACAGATTGTATAAGTATGCAACTAAGCTGAAGAAAAACTGTAAGGAAATAAC
TGATAAGGAATTGGACAAGAAAATGAAGGAGCTGGCCGCCGTCATGAGCGACCCTGACCTG
GAAACTGAGACTATGTGCCTCCACGACGACGAGTCGTGTCGCTACGAAGGGCAAGTCGCTG
TTTACCAGGATGTATACGCCGTCGACGGCCCCACCAGCCTGTACCACCAGGCCAACAAGGG
CGTGAGGGTGGCCTACTGGATCGGCTTCGACACCACACCCTTCATGTTCAAGAACCTGGCCG
GCGCCTACCCCAGCTACAGCACCAACTGGGCCGACGAGACAGTGCTGACCGCCAGGAACAT
CGGCCTGTGCAGCAGCGACGTGATGGAGAGGAGCCGGAGGGGCATGAGCATCCTGAGGAA
GAAGTACCTGAAGCCCAGCAACAACGTGCTGTTCAGCGTGGGCAGCACCATCTACCACGAG
AAGAGGGACCTGCTGAGGAGCTGGCACCTGCCCAGCGTGTTCCACCTGAGGGGCAAGCAGA
ACTACACCTGCAGGTGCGAGACAATCGTGAGCTGCGACGGCTACGTGGTGAAGAGGATCGC
CATCAGCCCCGGCCTGTACGGCAAGCCCAGCGGCTACGCCGCCACCATGCACAGGGAGGGC
TTCCTGTGCTGCAAGGTGACCGACACCCTGAACGGCGAGAGGGTGAGCTTCCCCGTGTGCA
CCTACGTGCCCGCCACCCTGTGCGACCAGATGACCGGCATCCTGGCCACCGACGTGAGCGC
CGACGACGCCCAGAAGCTGCTGGTGGGCCTGAACCAGAGGATCGTGGTGAACGGCAGGACC
CAGAGGAACACCAACACCATGAAGAACTACCTGCTGCCCGTGGTGGCCCAGGCCTTCGCCA
GGTGGGCCAAGGAGTACAAGGAGGACCAGGAGGACGAGAGGCCCCTGGGCCTGAGGGACC
GACAGCTGGTGATGGGCTGCTGCTGGGCCTTCAGGCGGCACAAGATCACCAGCATCTACAA
GAGGCCCGACACCCAGACCATCATCAAGGTGAACAGCGACTTCCACAGCTTCGTGCTGCCC
AGGATCGGCAGCAACACCCTGGAGATCGGCCTGAGGACCCGGATCAGGAAGATGCTGGAG
GAGCACAAGGAGCCCAGCCCTCTGATCACCGCCGAGGACGTGCAGGAGGCCAAGTGCGCC
GCCGACGAGGCCAAGGAGGTGAGGGAGGCCGAGGAGCTGAGGGCCGCCCTGCCTCCCCTG
GCCGCCGACGTGGAGGAGCCCACCCTGGAGGCCGACGTGGACCTGATGCTGCAGGAGGCCG
GCGCCGGCAGCGTGGAGACACCCAGGGGCCTGATCAAGGTGACCAGCTACGACGGCGAGG
ACAAGATCGGCAGCTACGCCGTGCTCAGCCCTCAGGCCGTGCTGAAGTCCGAGAAGCTGAG
CTGCATCCACCCTCTGGCCGAGCAGGTGATCGTGATCACCCACAGCGGCAGGAAGGGCAGG
TACGCCGTGGAGCCCTACCACGGCAAGGTGGTGGTCCCCGAGGGCCACGCCATCCCCGTGC
AGGACTTCCAGGCCCTGAGCGAGAGCGCCACCATCGTGTATAACGAGAGGGAGTTCGTGAA
CAGGTACCTGCACCACATCGCCACCCACGGCGGCGCCCTGAACACCGACGAGGAGTACTAC
AAGACCGTGAAGCCCAGCGAGCACGACGGCGAGTACCTGTACGACATCGACAGGAAGCAG
TGCGTGAAGAAGGAGCTGGTGACCGGCCTGGGCCTGACCGGCGAGCTGGTGGACCCTCCCT
TCCACGAGTTCGCCTACGAGAGCCTGAGGACCAGGCCCGCCGCTCCCTACCAGGTGCCCAC
CATCGGCGTGTACGGCGTGCCCGGCAGCGGCAAGAGCGGCATCATCAAGAGCGCCGTGACC
AAGAAGGACCTGGTGGTGAGCGCCAAGAAGGAGAACTGCGCCGAGATCATCAGGGACGTG
AAGAAGATGAAGGGCCTGGACGTGAACGCCAGGACCGTGGACAGCGTGCTCCTGAACGGCT
GCAAGCACCCCGTGGAGACACTGTATATCGACGAGGCCTTCGCCTGCCACGCCGGCACCCT
GAGGGCCCTGATCGCCATCATCAGGCCCAAGAAGGCCGTGCTGTGCGGCGACCCCAAGCAG
TGCGGCTTCTTCAACATGATGTGCCTGAAGGTGCACTTCAACCACGAGATCTGCACCCAGGT
GTTCCACAAGAGCATCAGCAGGCGGTGCACCAAGAGCGTGACCAGCGTGGTGAGCACCCTG
TTCTACGACAAGAAGATGAGGACCACCAACCCCAAGGAGACAAAGATCGTGATCGACACCA
CCGGCAGCACCAAGCCCAAGCAGGACGACCTGATCCTGACCTGCTTCAGGGGCTGGGTGAA
GCAGCTGCAGATCGACTACAAGGGCAACGAGATCATGACCGCCGCCGCTAGCCAGGGCCTG
ACCAGGAAGGGCGTGTACGCCGTGAGGTACAAGGTGAACGAGAATCCCCTGTACGCCCCTA
CCAGCGAGCACGTGAACGTCCTGCTGACCAGGACCGAGGACAGGATCGTGTGGAAGACCCT
GGCCGGCGACCCCTGGATCAAGACCCTGACCGCCAAGTACCCCGGCAACTTCACCGCCACC
ATCGAGGAGTGGCAGGCCGAGCACGACGCCATCATGAGGCACATCCTGGAGAGGCCCGACC
CCACCGACGTGTTCCAGAACAAGGCCAACGTGTGCTGGGCCAAGGCCCTGGTGCCCGTGCT
GAAGACCGCCGGCATCGACATGACCACCGAGCAGTGGAACACCGTGGACTACTTCGAGACA
GACAAGGCCCACAGCGCCGAGATCGTGCTGAACCAGCTGTGCGTGAGGTTCTTCGGCCTGG
ACCTGGACAGCGGCCTGTTCAGCGCCCCTACCGTGCCCCTGAGCATCAGGAACAACCACTG
GGACAACAGCCCCAGCCCCAACATGTACGGCCTGAACAAGGAGGTGGTGAGGCAGCTGAG
CAGGCGGTACCCTCAGCTGCCCAGGGCCGTGGCCACCGGCAGGGTGTACGACATGAACACC
GGCACCCTGAGGAACTACGACCCCAGGATCAACCTGGTGCCCGTGAACAGGCGGCTGCCAC
ACGCCCTGGTGCTGCACCACAACGAGCACCCTCAGAGCGACTTCAGCAGCTTCGTGAGCAA
GCTGAAGGGCAGGACCGTGCTGGTGGTGGGCGAGAAGCTGAGCGTGCCCGGCAAGATGGT
GGACTGGCTGAGCGACAGGCCCGAGGCCACCTTCCGGGCCAGGCTGGACCTGGGCATCCCC
GGCGACGTGCCCAAGTACGACATCATCTTCGTGAACGTGAGGACCCCTTACAAGTACCACC
ACTACCAGCAGTGCGAGGACCACGCCATCAAGCTGAGCATGCTGACCAAGAAGGCCTGCCT
GCACCTGAACCCCGGCGGCACCTGCGTGAGCATCGGCTACGGCTACGCCGACAGGGCCAGC
GAGAGCATCATCGGCGCCATCGCCAGGCTGTTCAAGTTCAGCAGGGTGTGCAAGCCCAAGA
GCAGCCTGGAGGAGACAGAGGTGCTGTTCGTGTTCATCGGCTACGACCGGAAGGCCAGGAC
CCACAACCCCTACAAGCTGAGCAGCACCCTGACCAACATCTACACCGGCAGCAGGCTGCAC
GAGGCCGGCTGCGCCCCTAGCTACCACGTGGTGAGGGGCGACATCGCCACCGCCACCGAGG
GCGTGATCATCAACGCCGCCAACAGCAAGGGCCAGCCCGGCGGCGGGGTGTGCGGCGCCCT
GTATAAGAAGTTCCCCGAGAGCTTCGACCTGCAGCCCATCGAGGTGGGCAAGGCCAGGCTG
GTGAAGGGCGCCGCCAAGCACATCATCCACGCCGTGGGCCCCAACTTCAACAAGGTGAGCG
AGGTGGAGGGCGACAAGCAGCTGGCCGAGGCCTACGAGAGCATCGCCAAGATCGTGAACG
ACAACAACTACAAGAGCGTGGCCATCCCTCTGCTGAGCACCGGCATCTTCAGCGGCAACAA
GGACAGGCTGACCCAGAGCCTGAACCACCTGCTGACCGCCCTGGACACCACCGACGCCGAC
GTGGCCATCTACTGCAGGGACAAGAAGTGGGAGATGACCCTGAAGGAGGCCGTGGCCAGG
CGGGAGGCCGTGGAGGAGATCTGCATCAGCGACGACAGCAGCGTGACGGAGCCCGACGCC
GAGCTGGTGAGGGTGCACCCCAAGAGCAGCCTGGCCGGCAGGAAGGGCTACAGCACCAGC
GACGGCAAGACCTTCAGCTACCTGGAGGGCACCAAGTTCCACCAGGCCGCCAAGGACATCG
CCGAGATCAACGCCATGTGGCCCGTGGCCACCGAGGCCAACGAGCAGGTGTGCATGTATAT
CCTGGGCGAGAGCATGAGCAGCATCAGGAGCAAGTGCCCCGTGGAGGAGAGCGAGGCCAG
CACCCCTCCCAGCACCCTGCCCTGCCTGTGCATCCACGCCATGACCCCTGAGAGGGTGCAGC
GGCTGAAGGCCAGCAGGCCCGAGCAGATCACCGTGTGCAGCAGCTTCCCTCTGCCCAAGTA
CCGGATCACCGGCGTGCAGAAGATCCAGTGCAGCCAGCCCATCCTGTTCAGCCCCAAGGTG
CCCGCCTACATCCACCCCAGGAAGTACCTGGTGGAGACACCCCCCGTGGACGAGACACCCG
AGCCCAGCGCCGAGAACCAGAGCACCGAGGGCACCCCTGAGCAGCCTCCCCTGATCACCGA
GGACGAGACAAGGACCAGGACGCCCGAGCCCATCATCATTGAGGAGGAAGAGGAGGACAG
CATCAGCCTGCTGAGCGACGGCCCCACCCACCAGGTGCTGCAGGTGGAGGCCGACATCCAC
GGCCCTCCCAGCGTGAGCAGCTCCAGCTGGAGCATCCCTCACGCCAGCGACTTCGACGTGG
ACAGCCTGAGCATCCTGGACACCCTGGAGGGCGCCAGCGTGACCAGCGGCGCCACCAGCGC
CGAGACAAACAGCTACTTCGCCAAGAGCATGGAGTTCCTGGCCAGGCCCGTGCCCGCCCCT
AGGACCGTGTTCAGGAACCCTCCCCACCCCGCCCCTAGGACCAGGACCCCTAGCCTGGCCC
CTAGCAGGGCCTGCAGCAGGACCAGCCTGGTGAGCACCCCTCCCGGCGTGAACCGGGTGAT
CACCAGGGAGGAGCTGGAGGCCCTGACCCCTAGCAGGACCCCTAGCAGGAGCGTGAGCAG
GACCAGCCTGGTGAGCAACCCTCCCGGCGTGAACCGGGTGATCACCAGGGAGGAGTTCGAG
GCCTTCGTGGCCCAGCAGCAAAGGCGGTTCGACGCCGGCGCCTACATCTTCAGCAGCGACA
CCGGCCAGGGCCACCTGCAGCAGAAGTCCGTGAGGCAGACCGTGCTGAGCGAGGTGGTCCT
GGAGAGGACGGAGCTGGAGATCAGCTACGCCCCTAGGCTGGACCAGGAGAAGGAGGAGCT
GCTGAGGAAGAAGCTGCAGCTGAACCCCACCCCTGCCAACAGGAGCAGGTACCAGAGCAG
GAAGGTGGAGAACATGAAGGCCATCACCGCCAGGCGGATCCTGCAGGGCCTGGGCCACTAC
CTGAAGGCCGAGGGCAAGGTGGAGTGCTACAGGACCCTGCACCCCGTGCCCCTGTACTCCA
GCTCCGTGAACAGGGCCTTCAGCAGCCCCAAGGTGGCCGTGGAGGCCTGCAACGCCATGCT
GAAGGAGAACTTCCCCACCGTGGCCAGCTACTGCATCATCCCCGAGTACGACGCCTACCTG
GACATGGTGGACGGCGCCAGCTGCTGCCTGGACACCGCCAGCTTCTGCCCCGCCAAGCTGA
GGAGCTTCCCCAAGAAGCACAGCTACCTGGAGCCCACCATCAGGAGCGCCGTGCCCAGCGC
CATCCAGAACACCCTGCAGAACGTGCTGGCCGCCGCTACCAAGAGGAACTGCAACGTGACC
CAGATGAGGGAGCTGCCCGTGCTGGACAGCGCCGCCTTCAACGTGGAGTGCTTCAAGAAGT
ACGCCTGCAACAACGAGTACTGGGAGACATTCAAGGAGAACCCCATCAGGCTGACCGAGGA
GAACGTGGTGAACTACATCACCAAGCTGAAGGGCCCCAAGGCCGCCGCTCTGTTCGCCAAG
ACCCACAACCTGAACATGCTCCAGGACATCCCTATGGACAGGTTCGTGATGGACCTGAAGA
GGGACGTGAAGGTGACCCCTGGCACCAAGCACACCGAGGAGAGGCCCAAGGTGCAGGTGA
TCCAGGCCGCCGACCCTCTGGCCACCGCCTACCTGTGCGGCATCCACAGGGAGCTGGTGAG
GCGGCTGAACGCCGTCCTGCTGCCCAACATCCACACCCTGTTCGACATGAGCGCCGAGGAC
TTCGACGCCATCATCGCCGAGCACTTCCAGCCCGGCGACTGCGTGCTGGAGACAGACATCG
CCAGCTTCGACAAGAGCGAGGACGACGCTATGGCCCTGACCGCCCTGATGATCCTGGAGGA
CCTGGGCGTGGACGCCGAGCTGCTGACCCTGATCGAGGCCGCCTTCGGCGAGATCAGCAGC
ATCCACCTGCCCACCAAGACCAAGTTCAAGTTCGGCGCCATGATGAAGTCCGGCATGTTCCT
GACCCTGTTCGTGAACACCGTGATCAACATCGTGATCGCCAGCAGGGTGCTGCGGGAGAGG
CTGACCGGCAGCCCCTGCGCCGCCTTCATCGGCGACGACAACATCGTGAAGGGCGTGAAGT
CCGACAAGCTGATGGCCGACAGGTGCGCCACCTGGCTGAACATGGAGGTGAAGATCATCGA
CGCCGTGGTGGGCGAGAAGGCCCCTTACTTCTGCGGCGGCTTCATCCTGTGCGACAGCGTGA
CCGGCACCGCCTGCAGGGTGGCCGACCCTCTGAAGAGGCTGTTCAAGCTGGGCAAGCCCCT
GGCCGCCGACGACGAGCACGACGACGATAGGCGGAGGGCCCTGCACGAGGAGAGCACCAG
GTGGAACCGGGTGGGCATCCTGAGCGAGCTGTGCAAGGCCGTGGAGAGCAGGTACGAGAC
AGTGGGCACCAGCATCATCGTGATGGCCATGACCACCCTGGCCAGCAGCGTCAAGTCCTTC
AGCTACCTGAGGGGGGCCCCTATAACTCTCTACGGCTAACCTGAATGGACTACGACATAGTC
TAGTCCGCCAAGGCCCTTAAGCTCCCTCCCCCCCCCCTAACGTTACTGGCCGAAGCCGCTTG
GAATAAGGCCGGTGTGCGTTTGTCTATATGTTATTTTCCACCATATTGCCGTCTTTTGGCAAT
GTGAGGGCCCGGAAACCTGGCCCTGTCTTCTTGACGAGCATTCCTAGGGGTCTTTCCCCTCT
CGCCAAAGGAATGCAAGGTCTGTTGAATGTCGTGAAGGAAGCAGTTCCTCTGGAAGCTTCTT
GAAGACAAACAACGTCTGTAGCGACCCTTTGCAGGCAGCGGAACCCCCCACCTGGCGACAG
GTGCCTCTGCGGCCAAAAGCCACGTGTATAAGATACACCTGCAAAGGCGGCACAACCCCAG
TGCCACGTTGTGAGTTGGATAGTTGTGGAAAGAGTCAAATGGCTCTCCTCAAGCGTATTCAA
CAAGGGGCTGAAGGATGCCCAGAAGGTACCCCATTGTATGGGATCTGATCTGGGGCCTCGG
TGCACATGCTTTACGTGTGTTTAGTCGAGGTTAAAAAACGTCTAGGCCCCCCGAACCACGGG
GACGTGGTTTTCCTTTGAAAAACACGATGATAATATGAATCCTAACCAGAAGATCATCACCA
TCGGCAGCGTGAGCCTGACCATCAGCACCATCTGCTTCTTCATGCAGATCGCCATCCTGATC
ACCACCGTGACCCTGCACTTCAAGCAGTACGAGTTCAACAGCCCTCCCAACAACCAGGTGA
TGCTGTGCGAGCCCACCATCATCGAGAGGAACATGACCGAGATCGTGTACCTGACCAACAC
CACCATCGAGAAGGAGATCTGCCCCAAGCCCGCCGAGTACAGGAACTGGAGCAAGCCCCA
GTGCGGCATCACCGGCTTCGCCCCTTTCAGCAAGGACAACAGCATCAGGCTGAGCGCCGGC
GGCGACATCTGGGTGACCAGGGAGCCCTACGTGAGCTGCGACCTGGACAAGTGCTACCAGT
TCGCCCTGGGCCAGGGCACCACCCTGAACAACGTGCACAGCAACAACACCGTGAGGGACAG
GACCCCTTACCGGACCCTGCTGATGAACGAGCTGGGCGTGCCCTTCCACCTGGGCACCAAG
CAGGTGTGCATCGCCTGGAGCAGCTCCAGCTGCCACGACGGCAAGGCCTGGCTGCACGTGT
GCATCACCGGCGACGACAAGAACGCCACCGCCAGCTTCATCTACAACGGCAGGCTGGTGGA
CAGCGTGGTGAGCTGGAGCAACGACATCCTGAGGACCCAGGAGAGCGAGTGCGTGTGCATC
AACGGCACCTGCACCGTGGTGATGACCGACGGCAACGCCACCGGCAAGGCCGACACCAAG
ATCCTGTTCATCGAGGAGGGCAAGATCGTGCACACCAGCAAGCTGAGCGGCAGCGCCCAGC
ACGTGGAGGAGTGCAGCTGCTACCCCAGGTACCCCGGCGTGAGGTGCGTGTGCAGGGACAA
CTGGAAGGGCAGCAACCGGCCCATCATCGACATCAACATCAAGGACCACAGCATCGTCAGC
AGGTACGTGTGCAGCGGCCTGGTGGGCGACACCCCTAGAAAGAGCGACAGCAGCTCCAGCA
GCCACTGCCTGAACCCCAACAACGAGAAGGGCGACCACGGCGTGAAGGGCTGGGCCTTCGA
CGACGGCAACGACGTGTGGATGGGCAGGACCATCAACGAGACAAGCAGGCTGGGCTACGA
GACATTCAAGGTGGTGGAGGGCTGGAGCAACCCCAAGAGCAAGCTGCAGATCAACAGGCA
GGTGATCGTGGACAGGGGCGACAGGAGCGGCTACAGCGGCATCTTCAGCGTGGAGGGCAA
GAGCTGCATCAACCGGTGCTTCTACGTGGAGCTGATCAGGGGCAGGAAGGAGGAGACAGA
GGTGCTGTGGACCAGCAACAGCATCGTGGTGTTCTGCGGCACCAGCGGCACCTACGGCACC
GGCAGCTGGCCCGACGGCGCCAACCTGAGCCTGATGCACATCTGATAGTAAACTCGAAGCA
GTGTTAAATCATTCAGCTACCTGAGAGGGGCCCCTATAACTCTCTACGGCTAACCTGAATGG
ACTACGACATAGTCTAGTCCGCCAAGGCCGAATTCCTCCCTCCCCCCCCCCTAACGTTACTG
GCCGAAGCCGCTTGGAATAAGGCCGGTGTGCGTTTGTCTATATGTTATTTTCCACCATATTG
CCGTCTTTTGGCAATGTGAGGGCCCGGAAACCTGGCCCTGTCTTCTTGACGAGCATTCCTAG
GGGTCTTTCCCCTCTCGCCAAAGGAATGCAAGGTCTGTTGAATGTCGTGAAGGAAGCAGTTC
CTCTGGAAGCTTCTTGAAGACAAACAACGTCTGTAGCGACCCTTTGCAGGCAGCGGAACCC
CCCACCTGGCGACAGGTGCCTCTGCGGCCAAAAGCCACGTGTATAAGATACACCTGCAAAG
GCGGCACAACCCCAGTGCCACGTTGTGAGTTGGATAGTTGTGGAAAGAGTCAAATGGCTCT
CCTCAAGCGTATTCAACAAGGGGCTGAAGGATGCCCAGAAGGTACCCCATTGTATGGGATC
TGATCTGGGGCCTCGGTGCACATGCTTTACGTGTGTTTAGTCGAGGTTAAAAAACGTCTAGG
CCCCCCGAACCACGGGGACGTGGTTTTCCTTTGAAAAACACGATGATAATATGAAGACCAT
CATCGCCCTGAGCTACATCCTGTGCCTGGTGTTCGCCCAGAAGATCCCCGGCAATGACAACA
GCACCGCCACCCTGTGCCTGGGCCACCACGCCGTGCCCAACGGCACCATCGTGAAGACCAT
CACCAACGACAGGATCGAGGTGACCAACGCCACCGAGCTGGTGCAGAACAGCAGCATCGG
CGAGATCTGCGACAGCCCTCACCAGATCCTGGACGGCGGCAACTGCACCCTGATCGACGCC
CTGCTGGGCGACCCTCAGTGCGACGGCTTCCAGAACAAGGAGTGGGACCTGTTCGTGGAGA
GGAGCAGGGCCAACAGCAACTGCTACCCCTACGACGTGCCCGACTACGCCAGCCTGAGGAG
CCTGGTGGCCAGCAGCGGCACCCTGGAGTTCAAGAACGAGAGCTTCAACTGGACCGGCGTG
AAGCAGAACGGCACCAGCAGCGCCTGCATCAGGGGCAGCAGCTCCAGCTTCTTCAGCCGGC
TGAATTGGCTCACCCACCTGAACTACACCTACCCCGCCCTGAACGTGACCATGCCCAACAAC
GAGCAGTTCGACAAGCTGTATATCTGGGGCGTGCACCACCCCAGCACCGACAAGGACCAGA
TCAGCCTGTTCGCCCAGCCCAGCGGCAGGATCACCGTGAGCACCAAGAGGAGCCAGCAGGC
CGTGATCCCCAACATCGGCAGCAGGCCCAGGATCAGGGACATCCCCAGCAGGATCAGCATC
TACTGGACCATCGTGAAGCCCGGCGACATCCTGCTGATCAACAGCACCGGCAACCTGATCG
CCCCTAGGGGCTACTTCAAGATCAGGAGCGGCAAGAGCAGCATCATGAGGAGCGACGCCCC
TATCGGCAAGTGCAAGAGCGAGTGCATCACCCCTAACGGCAGCATCCCCAACGACAAGCCC
TTCCAGAACGTGAACAGGATCACCTACGGCGCCTGCCCCAGGTACGTGAAGCAGAGCACCC
TGAAGCTGGCCACCGGCATGAGGAACGTGCCCGAGAAGCAGACCAGGGGCATCTTCGGCGC
CATCGCCGGCTTCATCGAGAACGGCTGGGAGGGCATGGTGGACGGCTGGTACGGCTTCAGG
CACCAGAACAGCGAGGGCAGGGGCCAGGCCGCCGACCTGAAGTCCACCCAGGCCGCCATC
GACCAGATCAACGGCAAGCTGAACAGGCTGATCGGCAAGACCAACGAGAAGTTCCACCAG
ATCGAGAAGGAGTTCAGCGAGGTGGAGGGCAGGGTGCAGGACCTGGAGAAGTACGTGGAG
GACACCAAGATCGACCTGTGGAGCTACAACGCCGAGCTGCTGGTGGCCCTGGAGAACCAGC
ACACCATCGACCTGACCGACAGCGAGATGAACAAGCTGTTCGAGAAGACCAAGAAGCAGCT
GAGGGAGAACGCCGAGGACATGGGCAACGGCTGCTTCAAGATCTACCACAAGTGCGACAA
CGCCTGCATCGGCAGCATCAGGAACGAGACATATGACCACAACGTGTACCGGGACGAGGCC
CTGAACAACCGGTTCCAGATCAAGGGCGTGGAGCTGAAGTCCGGCTACAAGGACTGGATCC
TGTGGATCAGCTTCGCCATGAGCTGCTTCCTGCTGTGCATCGCCCTGCTGGGCTTCATCATGT
GGGCCTGCCAGAAGGGCAACATCAGGTGCAACATCTGCATCTGATAGTAAACTCGAGTATG
TTACGTGCAAAGGTGATTGTCACCCCCCGAAAGACCATATTGTGACACACCCTCAGTATCAC
GCCCAAACATTTACAGCCGCGGTGTCAAAAACCGCGTGGACGTGGTTAACATCCCTGCTGG
GAGGATCAGCCGTAATTATTATAATTGGCTTGGTGCTGGCTACTATTGTGGCCATGTACGTG
CTGACCAACCAGAAACATAATTGAATACAGCAGCAATTGGCAAGCTGCTTACATAGAACTC
GCGGCGATTGGCATGCCGCCTTAAAATTTTTATTTTATTTTTTCTTTTCTTTTCCGAATCGGAT
TTTGTTTTTAATATTTC
SEQ ID NO: 155 (sgP EMCV NA sgP EV71 HA)
ATGGGCGGCGCATGAGAGAAGCCCAGACCAATTACCTACCCAAAATGGAGAAAGTTCACGT
TGACATCGAGGAAGACAGCCCATTCCTCAGAGCTTTGCAGCGGAGCTTCCCGCAGTTTGAG
GTAGAAGCCAAGCAGGTCACTGATAATGACCATGCTAATGCCAGAGCGTTTTCGCATCTGG
CTTCAAAACTGATCGAAACGGAGGTGGACCCATCCGACACGATCCTTGACATTGGAAGTGC
GCCCGCCCGCAGAATGTATTCTAAGCACAAGTATCATTGTATCTGTCCGATGAGATGTGCGG
AAGATCCGGACAGATTGTATAAGTATGCAACTAAGCTGAAGAAAAACTGTAAGGAAATAAC
TGATAAGGAATTGGACAAGAAAATGAAGGAGCTGGCCGCCGTCATGAGCGACCCTGACCTG
GAAACTGAGACTATGTGCCTCCACGACGACGAGTCGTGTCGCTACGAAGGGCAAGTCGCTG
TTTACCAGGATGTATACGCCGTCGACGGCCCCACCAGCCTGTACCACCAGGCCAACAAGGG
CGTGAGGGTGGCCTACTGGATCGGCTTCGACACCACACCCTTCATGTTCAAGAACCTGGCCG
GCGCCTACCCCAGCTACAGCACCAACTGGGCCGACGAGACAGTGCTGACCGCCAGGAACAT
CGGCCTGTGCAGCAGCGACGTGATGGAGAGGAGCCGGAGGGGCATGAGCATCCTGAGGAA
GAAGTACCTGAAGCCCAGCAACAACGTGCTGTTCAGCGTGGGCAGCACCATCTACCACGAG
AAGAGGGACCTGCTGAGGAGCTGGCACCTGCCCAGCGTGTTCCACCTGAGGGGCAAGCAGA
ACTACACCTGCAGGTGCGAGACAATCGTGAGCTGCGACGGCTACGTGGTGAAGAGGATCGC
CATCAGCCCCGGCCTGTACGGCAAGCCCAGCGGCTACGCCGCCACCATGCACAGGGAGGGC
TTCCTGTGCTGCAAGGTGACCGACACCCTGAACGGCGAGAGGGTGAGCTTCCCCGTGTGCA
CCTACGTGCCCGCCACCCTGTGCGACCAGATGACCGGCATCCTGGCCACCGACGTGAGCGC
CGACGACGCCCAGAAGCTGCTGGTGGGCCTGAACCAGAGGATCGTGGTGAACGGCAGGACC
CAGAGGAACACCAACACCATGAAGAACTACCTGCTGCCCGTGGTGGCCCAGGCCTTCGCCA
GGTGGGCCAAGGAGTACAAGGAGGACCAGGAGGACGAGAGGCCCCTGGGCCTGAGGGACC
GACAGCTGGTGATGGGCTGCTGCTGGGCCTTCAGGCGGCACAAGATCACCAGCATCTACAA
GAGGCCCGACACCCAGACCATCATCAAGGTGAACAGCGACTTCCACAGCTTCGTGCTGCCC
AGGATCGGCAGCAACACCCTGGAGATCGGCCTGAGGACCCGGATCAGGAAGATGCTGGAG
GAGCACAAGGAGCCCAGCCCTCTGATCACCGCCGAGGACGTGCAGGAGGCCAAGTGCGCC
GCCGACGAGGCCAAGGAGGTGAGGGAGGCCGAGGAGCTGAGGGCCGCCCTGCCTCCCCTG
GCCGCCGACGTGGAGGAGCCCACCCTGGAGGCCGACGTGGACCTGATGCTGCAGGAGGCCG
GCGCCGGCAGCGTGGAGACACCCAGGGGCCTGATCAAGGTGACCAGCTACGACGGCGAGG
ACAAGATCGGCAGCTACGCCGTGCTCAGCCCTCAGGCCGTGCTGAAGTCCGAGAAGCTGAG
CTGCATCCACCCTCTGGCCGAGCAGGTGATCGTGATCACCCACAGCGGCAGGAAGGGCAGG
TACGCCGTGGAGCCCTACCACGGCAAGGTGGTGGTCCCCGAGGGCCACGCCATCCCCGTGC
AGGACTTCCAGGCCCTGAGCGAGAGCGCCACCATCGTGTATAACGAGAGGGAGTTCGTGAA
CAGGTACCTGCACCACATCGCCACCCACGGCGGCGCCCTGAACACCGACGAGGAGTACTAC
AAGACCGTGAAGCCCAGCGAGCACGACGGCGAGTACCTGTACGACATCGACAGGAAGCAG
TGCGTGAAGAAGGAGCTGGTGACCGGCCTGGGCCTGACCGGCGAGCTGGTGGACCCTCCCT
TCCACGAGTTCGCCTACGAGAGCCTGAGGACCAGGCCCGCCGCTCCCTACCAGGTGCCCAC
CATCGGCGTGTACGGCGTGCCCGGCAGCGGCAAGAGCGGCATCATCAAGAGCGCCGTGACC
AAGAAGGACCTGGTGGTGAGCGCCAAGAAGGAGAACTGCGCCGAGATCATCAGGGACGTG
AAGAAGATGAAGGGCCTGGACGTGAACGCCAGGACCGTGGACAGCGTGCTCCTGAACGGCT
GCAAGCACCCCGTGGAGACACTGTATATCGACGAGGCCTTCGCCTGCCACGCCGGCACCCT
GAGGGCCCTGATCGCCATCATCAGGCCCAAGAAGGCCGTGCTGTGCGGCGACCCCAAGCAG
TGCGGCTTCTTCAACATGATGTGCCTGAAGGTGCACTTCAACCACGAGATCTGCACCCAGGT
GTTCCACAAGAGCATCAGCAGGCGGTGCACCAAGAGCGTGACCAGCGTGGTGAGCACCCTG
TTCTACGACAAGAAGATGAGGACCACCAACCCCAAGGAGACAAAGATCGTGATCGACACCA
CCGGCAGCACCAAGCCCAAGCAGGACGACCTGATCCTGACCTGCTTCAGGGGCTGGGTGAA
GCAGCTGCAGATCGACTACAAGGGCAACGAGATCATGACCGCCGCCGCTAGCCAGGGCCTG
ACCAGGAAGGGCGTGTACGCCGTGAGGTACAAGGTGAACGAGAATCCCCTGTACGCCCCTA
CCAGCGAGCACGTGAACGTCCTGCTGACCAGGACCGAGGACAGGATCGTGTGGAAGACCCT
GGCCGGCGACCCCTGGATCAAGACCCTGACCGCCAAGTACCCCGGCAACTTCACCGCCACC
ATCGAGGAGTGGCAGGCCGAGCACGACGCCATCATGAGGCACATCCTGGAGAGGCCCGACC
CCACCGACGTGTTCCAGAACAAGGCCAACGTGTGCTGGGCCAAGGCCCTGGTGCCCGTGCT
GAAGACCGCCGGCATCGACATGACCACCGAGCAGTGGAACACCGTGGACTACTTCGAGACA
GACAAGGCCCACAGCGCCGAGATCGTGCTGAACCAGCTGTGCGTGAGGTTCTTCGGCCTGG
ACCTGGACAGCGGCCTGTTCAGCGCCCCTACCGTGCCCCTGAGCATCAGGAACAACCACTG
GGACAACAGCCCCAGCCCCAACATGTACGGCCTGAACAAGGAGGTGGTGAGGCAGCTGAG
CAGGCGGTACCCTCAGCTGCCCAGGGCCGTGGCCACCGGCAGGGTGTACGACATGAACACC
GGCACCCTGAGGAACTACGACCCCAGGATCAACCTGGTGCCCGTGAACAGGCGGCTGCCAC
ACGCCCTGGTGCTGCACCACAACGAGCACCCTCAGAGCGACTTCAGCAGCTTCGTGAGCAA
GCTGAAGGGCAGGACCGTGCTGGTGGTGGGCGAGAAGCTGAGCGTGCCCGGCAAGATGGT
GGACTGGCTGAGCGACAGGCCCGAGGCCACCTTCCGGGCCAGGCTGGACCTGGGCATCCCC
GGCGACGTGCCCAAGTACGACATCATCTTCGTGAACGTGAGGACCCCTTACAAGTACCACC
ACTACCAGCAGTGCGAGGACCACGCCATCAAGCTGAGCATGCTGACCAAGAAGGCCTGCCT
GCACCTGAACCCCGGCGGCACCTGCGTGAGCATCGGCTACGGCTACGCCGACAGGGCCAGC
GAGAGCATCATCGGCGCCATCGCCAGGCTGTTCAAGTTCAGCAGGGTGTGCAAGCCCAAGA
GCAGCCTGGAGGAGACAGAGGTGCTGTTCGTGTTCATCGGCTACGACCGGAAGGCCAGGAC
CCACAACCCCTACAAGCTGAGCAGCACCCTGACCAACATCTACACCGGCAGCAGGCTGCAC
GAGGCCGGCTGCGCCCCTAGCTACCACGTGGTGAGGGGCGACATCGCCACCGCCACCGAGG
GCGTGATCATCAACGCCGCCAACAGCAAGGGCCAGCCCGGCGGCGGGGTGTGCGGCGCCCT
GTATAAGAAGTTCCCCGAGAGCTTCGACCTGCAGCCCATCGAGGTGGGCAAGGCCAGGCTG
GTGAAGGGCGCCGCCAAGCACATCATCCACGCCGTGGGCCCCAACTTCAACAAGGTGAGCG
AGGTGGAGGGCGACAAGCAGCTGGCCGAGGCCTACGAGAGCATCGCCAAGATCGTGAACG
ACAACAACTACAAGAGCGTGGCCATCCCTCTGCTGAGCACCGGCATCTTCAGCGGCAACAA
GGACAGGCTGACCCAGAGCCTGAACCACCTGCTGACCGCCCTGGACACCACCGACGCCGAC
GTGGCCATCTACTGCAGGGACAAGAAGTGGGAGATGACCCTGAAGGAGGCCGTGGCCAGG
CGGGAGGCCGTGGAGGAGATCTGCATCAGCGACGACAGCAGCGTGACGGAGCCCGACGCC
GAGCTGGTGAGGGTGCACCCCAAGAGCAGCCTGGCCGGCAGGAAGGGCTACAGCACCAGC
GACGGCAAGACCTTCAGCTACCTGGAGGGCACCAAGTTCCACCAGGCCGCCAAGGACATCG
CCGAGATCAACGCCATGTGGCCCGTGGCCACCGAGGCCAACGAGCAGGTGTGCATGTATAT
CCTGGGCGAGAGCATGAGCAGCATCAGGAGCAAGTGCCCCGTGGAGGAGAGCGAGGCCAG
CACCCCTCCCAGCACCCTGCCCTGCCTGTGCATCCACGCCATGACCCCTGAGAGGGTGCAGC
GGCTGAAGGCCAGCAGGCCCGAGCAGATCACCGTGTGCAGCAGCTTCCCTCTGCCCAAGTA
CCGGATCACCGGCGTGCAGAAGATCCAGTGCAGCCAGCCCATCCTGTTCAGCCCCAAGGTG
CCCGCCTACATCCACCCCAGGAAGTACCTGGTGGAGACACCCCCCGTGGACGAGACACCCG
AGCCCAGCGCCGAGAACCAGAGCACCGAGGGCACCCCTGAGCAGCCTCCCCTGATCACCGA
GGACGAGACAAGGACCAGGACGCCCGAGCCCATCATCATTGAGGAGGAAGAGGAGGACAG
CATCAGCCTGCTGAGCGACGGCCCCACCCACCAGGTGCTGCAGGTGGAGGCCGACATCCAC
GGCCCTCCCAGCGTGAGCAGCTCCAGCTGGAGCATCCCTCACGCCAGCGACTTCGACGTGG
ACAGCCTGAGCATCCTGGACACCCTGGAGGGCGCCAGCGTGACCAGCGGCGCCACCAGCGC
CGAGACAAACAGCTACTTCGCCAAGAGCATGGAGTTCCTGGCCAGGCCCGTGCCCGCCCCT
AGGACCGTGTTCAGGAACCCTCCCCACCCCGCCCCTAGGACCAGGACCCCTAGCCTGGCCC
CTAGCAGGGCCTGCAGCAGGACCAGCCTGGTGAGCACCCCTCCCGGCGTGAACCGGGTGAT
CACCAGGGAGGAGCTGGAGGCCCTGACCCCTAGCAGGACCCCTAGCAGGAGCGTGAGCAG
GACCAGCCTGGTGAGCAACCCTCCCGGCGTGAACCGGGTGATCACCAGGGAGGAGTTCGAG
GCCTTCGTGGCCCAGCAGCAAAGGCGGTTCGACGCCGGCGCCTACATCTTCAGCAGCGACA
CCGGCCAGGGCCACCTGCAGCAGAAGTCCGTGAGGCAGACCGTGCTGAGCGAGGTGGTCCT
GGAGAGGACGGAGCTGGAGATCAGCTACGCCCCTAGGCTGGACCAGGAGAAGGAGGAGCT
GCTGAGGAAGAAGCTGCAGCTGAACCCCACCCCTGCCAACAGGAGCAGGTACCAGAGCAG
GAAGGTGGAGAACATGAAGGCCATCACCGCCAGGCGGATCCTGCAGGGCCTGGGCCACTAC
CTGAAGGCCGAGGGCAAGGTGGAGTGCTACAGGACCCTGCACCCCGTGCCCCTGTACTCCA
GCTCCGTGAACAGGGCCTTCAGCAGCCCCAAGGTGGCCGTGGAGGCCTGCAACGCCATGCT
GAAGGAGAACTTCCCCACCGTGGCCAGCTACTGCATCATCCCCGAGTACGACGCCTACCTG
GACATGGTGGACGGCGCCAGCTGCTGCCTGGACACCGCCAGCTTCTGCCCCGCCAAGCTGA
GGAGCTTCCCCAAGAAGCACAGCTACCTGGAGCCCACCATCAGGAGCGCCGTGCCCAGCGC
CATCCAGAACACCCTGCAGAACGTGCTGGCCGCCGCTACCAAGAGGAACTGCAACGTGACC
CAGATGAGGGAGCTGCCCGTGCTGGACAGCGCCGCCTTCAACGTGGAGTGCTTCAAGAAGT
ACGCCTGCAACAACGAGTACTGGGAGACATTCAAGGAGAACCCCATCAGGCTGACCGAGGA
GAACGTGGTGAACTACATCACCAAGCTGAAGGGCCCCAAGGCCGCCGCTCTGTTCGCCAAG
ACCCACAACCTGAACATGCTCCAGGACATCCCTATGGACAGGTTCGTGATGGACCTGAAGA
GGGACGTGAAGGTGACCCCTGGCACCAAGCACACCGAGGAGAGGCCCAAGGTGCAGGTGA
TCCAGGCCGCCGACCCTCTGGCCACCGCCTACCTGTGCGGCATCCACAGGGAGCTGGTGAG
GCGGCTGAACGCCGTCCTGCTGCCCAACATCCACACCCTGTTCGACATGAGCGCCGAGGAC
TTCGACGCCATCATCGCCGAGCACTTCCAGCCCGGCGACTGCGTGCTGGAGACAGACATCG
CCAGCTTCGACAAGAGCGAGGACGACGCTATGGCCCTGACCGCCCTGATGATCCTGGAGGA
CCTGGGCGTGGACGCCGAGCTGCTGACCCTGATCGAGGCCGCCTTCGGCGAGATCAGCAGC
ATCCACCTGCCCACCAAGACCAAGTTCAAGTTCGGCGCCATGATGAAGTCCGGCATGTTCCT
GACCCTGTTCGTGAACACCGTGATCAACATCGTGATCGCCAGCAGGGTGCTGCGGGAGAGG
CTGACCGGCAGCCCCTGCGCCGCCTTCATCGGCGACGACAACATCGTGAAGGGCGTGAAGT
CCGACAAGCTGATGGCCGACAGGTGCGCCACCTGGCTGAACATGGAGGTGAAGATCATCGA
CGCCGTGGTGGGCGAGAAGGCCCCTTACTTCTGCGGCGGCTTCATCCTGTGCGACAGCGTGA
CCGGCACCGCCTGCAGGGTGGCCGACCCTCTGAAGAGGCTGTTCAAGCTGGGCAAGCCCCT
GGCCGCCGACGACGAGCACGACGACGATAGGCGGAGGGCCCTGCACGAGGAGAGCACCAG
GTGGAACCGGGTGGGCATCCTGAGCGAGCTGTGCAAGGCCGTGGAGAGCAGGTACGAGAC
AGTGGGCACCAGCATCATCGTGATGGCCATGACCACCCTGGCCAGCAGCGTCAAGTCCTTC
AGCTACCTGAGGGGGGCCCCTATAACTCTCTACGGCTAACCTGAATGGACTACGACATAGTC
TAGTCCGCCAAGGCCCTTAAGCTCCCTCCCCCCCCCCTAACGTTACTGGCCGAAGCCGCTTG
GAATAAGGCCGGTGTGCGTTTGTCTATATGTTATTTTCCACCATATTGCCGTCTTTTGGCAAT
GTGAGGGCCCGGAAACCTGGCCCTGTCTTCTTGACGAGCATTCCTAGGGGTCTTTCCCCTCT
CGCCAAAGGAATGCAAGGTCTGTTGAATGTCGTGAAGGAAGCAGTTCCTCTGGAAGCTTCTT
GAAGACAAACAACGTCTGTAGCGACCCTTTGCAGGCAGCGGAACCCCCCACCTGGCGACAG
GTGCCTCTGCGGCCAAAAGCCACGTGTATAAGATACACCTGCAAAGGCGGCACAACCCCAG
TGCCACGTTGTGAGTTGGATAGTTGTGGAAAGAGTCAAATGGCTCTCCTCAAGCGTATTCAA
CAAGGGGCTGAAGGATGCCCAGAAGGTACCCCATTGTATGGGATCTGATCTGGGGCCTCGG
TGCACATGCTTTACGTGTGTTTAGTCGAGGTTAAAAAACGTCTAGGCCCCCCGAACCACGGG
GACGTGGTTTTCCTTTGAAAAACACGATGATAATATGAATCCTAACCAGAAGATCATCACCA
TCGGCAGCGTGAGCCTGACCATCAGCACCATCTGCTTCTTCATGCAGATCGCCATCCTGATC
ACCACCGTGACCCTGCACTTCAAGCAGTACGAGTTCAACAGCCCTCCCAACAACCAGGTGA
TGCTGTGCGAGCCCACCATCATCGAGAGGAACATGACCGAGATCGTGTACCTGACCAACAC
CACCATCGAGAAGGAGATCTGCCCCAAGCCCGCCGAGTACAGGAACTGGAGCAAGCCCCA
GTGCGGCATCACCGGCTTCGCCCCTTTCAGCAAGGACAACAGCATCAGGCTGAGCGCCGGC
GGCGACATCTGGGTGACCAGGGAGCCCTACGTGAGCTGCGACCTGGACAAGTGCTACCAGT
TCGCCCTGGGCCAGGGCACCACCCTGAACAACGTGCACAGCAACAACACCGTGAGGGACAG
GACCCCTTACCGGACCCTGCTGATGAACGAGCTGGGCGTGCCCTTCCACCTGGGCACCAAG
CAGGTGTGCATCGCCTGGAGCAGCTCCAGCTGCCACGACGGCAAGGCCTGGCTGCACGTGT
GCATCACCGGCGACGACAAGAACGCCACCGCCAGCTTCATCTACAACGGCAGGCTGGTGGA
CAGCGTGGTGAGCTGGAGCAACGACATCCTGAGGACCCAGGAGAGCGAGTGCGTGTGCATC
AACGGCACCTGCACCGTGGTGATGACCGACGGCAACGCCACCGGCAAGGCCGACACCAAG
ATCCTGTTCATCGAGGAGGGCAAGATCGTGCACACCAGCAAGCTGAGCGGCAGCGCCCAGC
ACGTGGAGGAGTGCAGCTGCTACCCCAGGTACCCCGGCGTGAGGTGCGTGTGCAGGGACAA
CTGGAAGGGCAGCAACCGGCCCATCATCGACATCAACATCAAGGACCACAGCATCGTCAGC
AGGTACGTGTGCAGCGGCCTGGTGGGCGACACCCCTAGAAAGAGCGACAGCAGCTCCAGCA
GCCACTGCCTGAACCCCAACAACGAGAAGGGCGACCACGGCGTGAAGGGCTGGGCCTTCGA
CGACGGCAACGACGTGTGGATGGGCAGGACCATCAACGAGACAAGCAGGCTGGGCTACGA
GACATTCAAGGTGGTGGAGGGCTGGAGCAACCCCAAGAGCAAGCTGCAGATCAACAGGCA
GGTGATCGTGGACAGGGGCGACAGGAGCGGCTACAGCGGCATCTTCAGCGTGGAGGGCAA
GAGCTGCATCAACCGGTGCTTCTACGTGGAGCTGATCAGGGGCAGGAAGGAGGAGACAGA
GGTGCTGTGGACCAGCAACAGCATCGTGGTGTTCTGCGGCACCAGCGGCACCTACGGCACC
GGCAGCTGGCCCGACGGCGCCAACCTGAGCCTGATGCACATCTGATAGTAAACTCGAAGCA
GTGTTAAATCATTCAGCTACCTGAGAGGGGCCCCTATAACTCTCTACGGCTAACCTGAATGG
ACTACGACATAGTCTAGTCCGCCAAGGCCGAATTCTTAAAACAGCTGTGGGTTGTCACCCAC
CCACAGGGTCCACTGGGCGCTAGTACACTGGTATCTCGGTACCTTTGTACGCCTGTTTTATA
CCCCCTCCCTGATTTGCAACTTAGAAGCAACGCAAACCAGATCAATAGTAGGTGTGACATAC
CAGTCGCATCTTGATCAAGCACTTCTGTATCCCCGGACCGAGTATCAATAGACTGTGCACAC
GGTTGAAGGAGAAAACGTCCGTTACCCGGCTAACTACTTCGAGAAGCCTAGTAACGCCATT
GAAGTTGCAGAGTGTTTCGCTCAGCACTCCCCCCGTGTAGATCAGGTCGATGAGTCACCGCA
TTCCCCACGGGCGACCGTGGCGGTGGCTGCGTTGGCGGCCTGCCTATGGGGTAACCCATAG
GACGCTCTAATACGGACATGGCGTGAAGAGTCTATTGAGCTAGTTAGTAGTCCTCCGGCCCC
TGAATGCGGCTAATCCTAACTGCGGAGCACATACCCTTAATCCAAAGGGCAGTGTGTCGTA
ACGGGCAACTCTGCAGCGGAACCGACTACTTTGGGTGTCCGTGTTTCTTTTTATTCTTGTATT
GGCTGCTTATGGTGACAATTAAAGAATTGTTACCATATAGCTATTGGATTGGCCATCCAGTG
TCAAACAGAGCTATTGTATATCTCTTTGTTGGATTCACACCTCTCACTCTTGAAACGTTACAC
ACCCTCAATTACATTATACTGCTGAACACGAAGCGATGAAGACCATCATCGCCCTGAGCTAC
ATCCTGTGCCTGGTGTTCGCCCAGAAGATCCCCGGCAATGACAACAGCACCGCCACCCTGTG
CCTGGGCCACCACGCCGTGCCCAACGGCACCATCGTGAAGACCATCACCAACGACAGGATC
GAGGTGACCAACGCCACCGAGCTGGTGCAGAACAGCAGCATCGGCGAGATCTGCGACAGCC
CTCACCAGATCCTGGACGGCGGCAACTGCACCCTGATCGACGCCCTGCTGGGCGACCCTCA
GTGCGACGGCTTCCAGAACAAGGAGTGGGACCTGTTCGTGGAGAGGAGCAGGGCCAACAG
CAACTGCTACCCCTACGACGTGCCCGACTACGCCAGCCTGAGGAGCCTGGTGGCCAGCAGC
GGCACCCTGGAGTTCAAGAACGAGAGCTTCAACTGGACCGGCGTGAAGCAGAACGGCACCA
GCAGCGCCTGCATCAGGGGCAGCAGCTCCAGCTTCTTCAGCCGGCTGAATTGGCTCACCCAC
CTGAACTACACCTACCCCGCCCTGAACGTGACCATGCCCAACAACGAGCAGTTCGACAAGC
TGTATATCTGGGGCGTGCACCACCCCAGCACCGACAAGGACCAGATCAGCCTGTTCGCCCA
GCCCAGCGGCAGGATCACCGTGAGCACCAAGAGGAGCCAGCAGGCCGTGATCCCCAACATC
GGCAGCAGGCCCAGGATCAGGGACATCCCCAGCAGGATCAGCATCTACTGGACCATCGTGA
AGCCCGGCGACATCCTGCTGATCAACAGCACCGGCAACCTGATCGCCCCTAGGGGCTACTT
CAAGATCAGGAGCGGCAAGAGCAGCATCATGAGGAGCGACGCCCCTATCGGCAAGTGCAA
GAGCGAGTGCATCACCCCTAACGGCAGCATCCCCAACGACAAGCCCTTCCAGAACGTGAAC
AGGATCACCTACGGCGCCTGCCCCAGGTACGTGAAGCAGAGCACCCTGAAGCTGGCCACCG
GCATGAGGAACGTGCCCGAGAAGCAGACCAGGGGCATCTTCGGCGCCATCGCCGGCTTCAT
CGAGAACGGCTGGGAGGGCATGGTGGACGGCTGGTACGGCTTCAGGCACCAGAACAGCGA
GGGCAGGGGCCAGGCCGCCGACCTGAAGTCCACCCAGGCCGCCATCGACCAGATCAACGGC
AAGCTGAACAGGCTGATCGGCAAGACCAACGAGAAGTTCCACCAGATCGAGAAGGAGTTC
AGCGAGGTGGAGGGCAGGGTGCAGGACCTGGAGAAGTACGTGGAGGACACCAAGATCGAC
CTGTGGAGCTACAACGCCGAGCTGCTGGTGGCCCTGGAGAACCAGCACACCATCGACCTGA
CCGACAGCGAGATGAACAAGCTGTTCGAGAAGACCAAGAAGCAGCTGAGGGAGAACGCCG
AGGACATGGGCAACGGCTGCTTCAAGATCTACCACAAGTGCGACAACGCCTGCATCGGCAG
CATCAGGAACGAGACATATGACCACAACGTGTACCGGGACGAGGCCCTGAACAACCGGTTC
CAGATCAAGGGCGTGGAGCTGAAGTCCGGCTACAAGGACTGGATCCTGTGGATCAGCTTCG
CCATGAGCTGCTTCCTGCTGTGCATCGCCCTGCTGGGCTTCATCATGTGGGCCTGCCAGAAG
GGCAACATCAGGTGCAACATCTGCATCTGATAGTAAACTCGAGTATGTTACGTGCAAAGGT
GATTGTCACCCCCCGAAAGACCATATTGTGACACACCCTCAGTATCACGCCCAAACATTTAC
AGCCGCGGTGTCAAAAACCGCGTGGACGTGGTTAACATCCCTGCTGGGAGGATCAGCCGTA
ATTATTATAATTGGCTTGGTGCTGGCTACTATTGTGGCCATGTACGTGCTGACCAACCAGAA
ACATAATTGAATACAGCAGCAATTGGCAAGCTGCTTACATAGAACTCGCGGCGATTGGCAT
GCCGCCTTAAAATTTTTATTTTATTTTTTCTTTTCTTTTCCGAATCGGATTTTGTTTTTAATATT
TC
SEQ ID NO: 156 (sgP EV71 NA sgP CVB3 HA)
ATGGGCGGCGCATGAGAGAAGCCCAGACCAATTACCTACCCAAAATGGAGAAAGTTCACGT
TGACATCGAGGAAGACAGCCCATTCCTCAGAGCTTTGCAGCGGAGCTTCCCGCAGTTTGAG
GTAGAAGCCAAGCAGGTCACTGATAATGACCATGCTAATGCCAGAGCGTTTTCGCATCTGG
CTTCAAAACTGATCGAAACGGAGGTGGACCCATCCGACACGATCCTTGACATTGGAAGTGC
GCCCGCCCGCAGAATGTATTCTAAGCACAAGTATCATTGTATCTGTCCGATGAGATGTGCGG
AAGATCCGGACAGATTGTATAAGTATGCAACTAAGCTGAAGAAAAACTGTAAGGAAATAAC
TGATAAGGAATTGGACAAGAAAATGAAGGAGCTGGCCGCCGTCATGAGCGACCCTGACCTG
GAAACTGAGACTATGTGCCTCCACGACGACGAGTCGTGTCGCTACGAAGGGCAAGTCGCTG
TTTACCAGGATGTATACGCCGTCGACGGCCCCACCAGCCTGTACCACCAGGCCAACAAGGG
CGTGAGGGTGGCCTACTGGATCGGCTTCGACACCACACCCTTCATGTTCAAGAACCTGGCCG
GCGCCTACCCCAGCTACAGCACCAACTGGGCCGACGAGACAGTGCTGACCGCCAGGAACAT
CGGCCTGTGCAGCAGCGACGTGATGGAGAGGAGCCGGAGGGGCATGAGCATCCTGAGGAA
GAAGTACCTGAAGCCCAGCAACAACGTGCTGTTCAGCGTGGGCAGCACCATCTACCACGAG
AAGAGGGACCTGCTGAGGAGCTGGCACCTGCCCAGCGTGTTCCACCTGAGGGGCAAGCAGA
ACTACACCTGCAGGTGCGAGACAATCGTGAGCTGCGACGGCTACGTGGTGAAGAGGATCGC
CATCAGCCCCGGCCTGTACGGCAAGCCCAGCGGCTACGCCGCCACCATGCACAGGGAGGGC
TTCCTGTGCTGCAAGGTGACCGACACCCTGAACGGCGAGAGGGTGAGCTTCCCCGTGTGCA
CCTACGTGCCCGCCACCCTGTGCGACCAGATGACCGGCATCCTGGCCACCGACGTGAGCGC
CGACGACGCCCAGAAGCTGCTGGTGGGCCTGAACCAGAGGATCGTGGTGAACGGCAGGACC
CAGAGGAACACCAACACCATGAAGAACTACCTGCTGCCCGTGGTGGCCCAGGCCTTCGCCA
GGTGGGCCAAGGAGTACAAGGAGGACCAGGAGGACGAGAGGCCCCTGGGCCTGAGGGACC
GACAGCTGGTGATGGGCTGCTGCTGGGCCTTCAGGCGGCACAAGATCACCAGCATCTACAA
GAGGCCCGACACCCAGACCATCATCAAGGTGAACAGCGACTTCCACAGCTTCGTGCTGCCC
AGGATCGGCAGCAACACCCTGGAGATCGGCCTGAGGACCCGGATCAGGAAGATGCTGGAG
GAGCACAAGGAGCCCAGCCCTCTGATCACCGCCGAGGACGTGCAGGAGGCCAAGTGCGCC
GCCGACGAGGCCAAGGAGGTGAGGGAGGCCGAGGAGCTGAGGGCCGCCCTGCCTCCCCTG
GCCGCCGACGTGGAGGAGCCCACCCTGGAGGCCGACGTGGACCTGATGCTGCAGGAGGCCG
GCGCCGGCAGCGTGGAGACACCCAGGGGCCTGATCAAGGTGACCAGCTACGACGGCGAGG
ACAAGATCGGCAGCTACGCCGTGCTCAGCCCTCAGGCCGTGCTGAAGTCCGAGAAGCTGAG
CTGCATCCACCCTCTGGCCGAGCAGGTGATCGTGATCACCCACAGCGGCAGGAAGGGCAGG
TACGCCGTGGAGCCCTACCACGGCAAGGTGGTGGTCCCCGAGGGCCACGCCATCCCCGTGC
AGGACTTCCAGGCCCTGAGCGAGAGCGCCACCATCGTGTATAACGAGAGGGAGTTCGTGAA
CAGGTACCTGCACCACATCGCCACCCACGGCGGCGCCCTGAACACCGACGAGGAGTACTAC
AAGACCGTGAAGCCCAGCGAGCACGACGGCGAGTACCTGTACGACATCGACAGGAAGCAG
TGCGTGAAGAAGGAGCTGGTGACCGGCCTGGGCCTGACCGGCGAGCTGGTGGACCCTCCCT
TCCACGAGTTCGCCTACGAGAGCCTGAGGACCAGGCCCGCCGCTCCCTACCAGGTGCCCAC
CATCGGCGTGTACGGCGTGCCCGGCAGCGGCAAGAGCGGCATCATCAAGAGCGCCGTGACC
AAGAAGGACCTGGTGGTGAGCGCCAAGAAGGAGAACTGCGCCGAGATCATCAGGGACGTG
AAGAAGATGAAGGGCCTGGACGTGAACGCCAGGACCGTGGACAGCGTGCTCCTGAACGGCT
GCAAGCACCCCGTGGAGACACTGTATATCGACGAGGCCTTCGCCTGCCACGCCGGCACCCT
GAGGGCCCTGATCGCCATCATCAGGCCCAAGAAGGCCGTGCTGTGCGGCGACCCCAAGCAG
TGCGGCTTCTTCAACATGATGTGCCTGAAGGTGCACTTCAACCACGAGATCTGCACCCAGGT
GTTCCACAAGAGCATCAGCAGGCGGTGCACCAAGAGCGTGACCAGCGTGGTGAGCACCCTG
TTCTACGACAAGAAGATGAGGACCACCAACCCCAAGGAGACAAAGATCGTGATCGACACCA
CCGGCAGCACCAAGCCCAAGCAGGACGACCTGATCCTGACCTGCTTCAGGGGCTGGGTGAA
GCAGCTGCAGATCGACTACAAGGGCAACGAGATCATGACCGCCGCCGCTAGCCAGGGCCTG
ACCAGGAAGGGCGTGTACGCCGTGAGGTACAAGGTGAACGAGAATCCCCTGTACGCCCCTA
CCAGCGAGCACGTGAACGTCCTGCTGACCAGGACCGAGGACAGGATCGTGTGGAAGACCCT
GGCCGGCGACCCCTGGATCAAGACCCTGACCGCCAAGTACCCCGGCAACTTCACCGCCACC
ATCGAGGAGTGGCAGGCCGAGCACGACGCCATCATGAGGCACATCCTGGAGAGGCCCGACC
CCACCGACGTGTTCCAGAACAAGGCCAACGTGTGCTGGGCCAAGGCCCTGGTGCCCGTGCT
GAAGACCGCCGGCATCGACATGACCACCGAGCAGTGGAACACCGTGGACTACTTCGAGACA
GACAAGGCCCACAGCGCCGAGATCGTGCTGAACCAGCTGTGCGTGAGGTTCTTCGGCCTGG
ACCTGGACAGCGGCCTGTTCAGCGCCCCTACCGTGCCCCTGAGCATCAGGAACAACCACTG
GGACAACAGCCCCAGCCCCAACATGTACGGCCTGAACAAGGAGGTGGTGAGGCAGCTGAG
CAGGCGGTACCCTCAGCTGCCCAGGGCCGTGGCCACCGGCAGGGTGTACGACATGAACACC
GGCACCCTGAGGAACTACGACCCCAGGATCAACCTGGTGCCCGTGAACAGGCGGCTGCCAC
ACGCCCTGGTGCTGCACCACAACGAGCACCCTCAGAGCGACTTCAGCAGCTTCGTGAGCAA
GCTGAAGGGCAGGACCGTGCTGGTGGTGGGCGAGAAGCTGAGCGTGCCCGGCAAGATGGT
GGACTGGCTGAGCGACAGGCCCGAGGCCACCTTCCGGGCCAGGCTGGACCTGGGCATCCCC
GGCGACGTGCCCAAGTACGACATCATCTTCGTGAACGTGAGGACCCCTTACAAGTACCACC
ACTACCAGCAGTGCGAGGACCACGCCATCAAGCTGAGCATGCTGACCAAGAAGGCCTGCCT
GCACCTGAACCCCGGCGGCACCTGCGTGAGCATCGGCTACGGCTACGCCGACAGGGCCAGC
GAGAGCATCATCGGCGCCATCGCCAGGCTGTTCAAGTTCAGCAGGGTGTGCAAGCCCAAGA
GCAGCCTGGAGGAGACAGAGGTGCTGTTCGTGTTCATCGGCTACGACCGGAAGGCCAGGAC
CCACAACCCCTACAAGCTGAGCAGCACCCTGACCAACATCTACACCGGCAGCAGGCTGCAC
GAGGCCGGCTGCGCCCCTAGCTACCACGTGGTGAGGGGCGACATCGCCACCGCCACCGAGG
GCGTGATCATCAACGCCGCCAACAGCAAGGGCCAGCCCGGCGGCGGGGTGTGCGGCGCCCT
GTATAAGAAGTTCCCCGAGAGCTTCGACCTGCAGCCCATCGAGGTGGGCAAGGCCAGGCTG
GTGAAGGGCGCCGCCAAGCACATCATCCACGCCGTGGGCCCCAACTTCAACAAGGTGAGCG
AGGTGGAGGGCGACAAGCAGCTGGCCGAGGCCTACGAGAGCATCGCCAAGATCGTGAACG
ACAACAACTACAAGAGCGTGGCCATCCCTCTGCTGAGCACCGGCATCTTCAGCGGCAACAA
GGACAGGCTGACCCAGAGCCTGAACCACCTGCTGACCGCCCTGGACACCACCGACGCCGAC
GTGGCCATCTACTGCAGGGACAAGAAGTGGGAGATGACCCTGAAGGAGGCCGTGGCCAGG
CGGGAGGCCGTGGAGGAGATCTGCATCAGCGACGACAGCAGCGTGACGGAGCCCGACGCC
GAGCTGGTGAGGGTGCACCCCAAGAGCAGCCTGGCCGGCAGGAAGGGCTACAGCACCAGC
GACGGCAAGACCTTCAGCTACCTGGAGGGCACCAAGTTCCACCAGGCCGCCAAGGACATCG
CCGAGATCAACGCCATGTGGCCCGTGGCCACCGAGGCCAACGAGCAGGTGTGCATGTATAT
CCTGGGCGAGAGCATGAGCAGCATCAGGAGCAAGTGCCCCGTGGAGGAGAGCGAGGCCAG
CACCCCTCCCAGCACCCTGCCCTGCCTGTGCATCCACGCCATGACCCCTGAGAGGGTGCAGC
GGCTGAAGGCCAGCAGGCCCGAGCAGATCACCGTGTGCAGCAGCTTCCCTCTGCCCAAGTA
CCGGATCACCGGCGTGCAGAAGATCCAGTGCAGCCAGCCCATCCTGTTCAGCCCCAAGGTG
CCCGCCTACATCCACCCCAGGAAGTACCTGGTGGAGACACCCCCCGTGGACGAGACACCCG
AGCCCAGCGCCGAGAACCAGAGCACCGAGGGCACCCCTGAGCAGCCTCCCCTGATCACCGA
GGACGAGACAAGGACCAGGACGCCCGAGCCCATCATCATTGAGGAGGAAGAGGAGGACAG
CATCAGCCTGCTGAGCGACGGCCCCACCCACCAGGTGCTGCAGGTGGAGGCCGACATCCAC
GGCCCTCCCAGCGTGAGCAGCTCCAGCTGGAGCATCCCTCACGCCAGCGACTTCGACGTGG
ACAGCCTGAGCATCCTGGACACCCTGGAGGGCGCCAGCGTGACCAGCGGCGCCACCAGCGC
CGAGACAAACAGCTACTTCGCCAAGAGCATGGAGTTCCTGGCCAGGCCCGTGCCCGCCCCT
AGGACCGTGTTCAGGAACCCTCCCCACCCCGCCCCTAGGACCAGGACCCCTAGCCTGGCCC
CTAGCAGGGCCTGCAGCAGGACCAGCCTGGTGAGCACCCCTCCCGGCGTGAACCGGGTGAT
CACCAGGGAGGAGCTGGAGGCCCTGACCCCTAGCAGGACCCCTAGCAGGAGCGTGAGCAG
GACCAGCCTGGTGAGCAACCCTCCCGGCGTGAACCGGGTGATCACCAGGGAGGAGTTCGAG
GCCTTCGTGGCCCAGCAGCAAAGGCGGTTCGACGCCGGCGCCTACATCTTCAGCAGCGACA
CCGGCCAGGGCCACCTGCAGCAGAAGTCCGTGAGGCAGACCGTGCTGAGCGAGGTGGTCCT
GGAGAGGACGGAGCTGGAGATCAGCTACGCCCCTAGGCTGGACCAGGAGAAGGAGGAGCT
GCTGAGGAAGAAGCTGCAGCTGAACCCCACCCCTGCCAACAGGAGCAGGTACCAGAGCAG
GAAGGTGGAGAACATGAAGGCCATCACCGCCAGGCGGATCCTGCAGGGCCTGGGCCACTAC
CTGAAGGCCGAGGGCAAGGTGGAGTGCTACAGGACCCTGCACCCCGTGCCCCTGTACTCCA
GCTCCGTGAACAGGGCCTTCAGCAGCCCCAAGGTGGCCGTGGAGGCCTGCAACGCCATGCT
GAAGGAGAACTTCCCCACCGTGGCCAGCTACTGCATCATCCCCGAGTACGACGCCTACCTG
GACATGGTGGACGGCGCCAGCTGCTGCCTGGACACCGCCAGCTTCTGCCCCGCCAAGCTGA
GGAGCTTCCCCAAGAAGCACAGCTACCTGGAGCCCACCATCAGGAGCGCCGTGCCCAGCGC
CATCCAGAACACCCTGCAGAACGTGCTGGCCGCCGCTACCAAGAGGAACTGCAACGTGACC
CAGATGAGGGAGCTGCCCGTGCTGGACAGCGCCGCCTTCAACGTGGAGTGCTTCAAGAAGT
ACGCCTGCAACAACGAGTACTGGGAGACATTCAAGGAGAACCCCATCAGGCTGACCGAGGA
GAACGTGGTGAACTACATCACCAAGCTGAAGGGCCCCAAGGCCGCCGCTCTGTTCGCCAAG
ACCCACAACCTGAACATGCTCCAGGACATCCCTATGGACAGGTTCGTGATGGACCTGAAGA
GGGACGTGAAGGTGACCCCTGGCACCAAGCACACCGAGGAGAGGCCCAAGGTGCAGGTGA
TCCAGGCCGCCGACCCTCTGGCCACCGCCTACCTGTGCGGCATCCACAGGGAGCTGGTGAG
GCGGCTGAACGCCGTCCTGCTGCCCAACATCCACACCCTGTTCGACATGAGCGCCGAGGAC
TTCGACGCCATCATCGCCGAGCACTTCCAGCCCGGCGACTGCGTGCTGGAGACAGACATCG
CCAGCTTCGACAAGAGCGAGGACGACGCTATGGCCCTGACCGCCCTGATGATCCTGGAGGA
CCTGGGCGTGGACGCCGAGCTGCTGACCCTGATCGAGGCCGCCTTCGGCGAGATCAGCAGC
ATCCACCTGCCCACCAAGACCAAGTTCAAGTTCGGCGCCATGATGAAGTCCGGCATGTTCCT
GACCCTGTTCGTGAACACCGTGATCAACATCGTGATCGCCAGCAGGGTGCTGCGGGAGAGG
CTGACCGGCAGCCCCTGCGCCGCCTTCATCGGCGACGACAACATCGTGAAGGGCGTGAAGT
CCGACAAGCTGATGGCCGACAGGTGCGCCACCTGGCTGAACATGGAGGTGAAGATCATCGA
CGCCGTGGTGGGCGAGAAGGCCCCTTACTTCTGCGGCGGCTTCATCCTGTGCGACAGCGTGA
CCGGCACCGCCTGCAGGGTGGCCGACCCTCTGAAGAGGCTGTTCAAGCTGGGCAAGCCCCT
GGCCGCCGACGACGAGCACGACGACGATAGGCGGAGGGCCCTGCACGAGGAGAGCACCAG
GTGGAACCGGGTGGGCATCCTGAGCGAGCTGTGCAAGGCCGTGGAGAGCAGGTACGAGAC
AGTGGGCACCAGCATCATCGTGATGGCCATGACCACCCTGGCCAGCAGCGTCAAGTCCTTC
AGCTACCTGAGGGGGGCCCCTATAACTCTCTACGGCTAACCTGAATGGACTACGACATAGTC
TAGTCCGCCAAGGCCCTTAAGTTAAAACAGCTGTGGGTTGTCACCCACCCACAGGGTCCACT
GGGCGCTAGTACACTGGTATCTCGGTACCTTTGTACGCCTGTTTTATACCCCCTCCCTGATTT
GCAACTTAGAAGCAACGCAAACCAGATCAATAGTAGGTGTGACATACCAGTCGCATCTTGA
TCAAGCACTTCTGTATCCCCGGACCGAGTATCAATAGACTGTGCACACGGTTGAAGGAGAA
AACGTCCGTTACCCGGCTAACTACTTCGAGAAGCCTAGTAACGCCATTGAAGTTGCAGAGTG
TTTCGCTCAGCACTCCCCCCGTGTAGATCAGGTCGATGAGTCACCGCATTCCCCACGGGCGA
CCGTGGCGGTGGCTGCGTTGGCGGCCTGCCTATGGGGTAACCCATAGGACGCTCTAATACG
GACATGGCGTGAAGAGTCTATTGAGCTAGTTAGTAGTCCTCCGGCCCCTGAATGCGGCTAAT
CCTAACTGCGGAGCACATACCCTTAATCCAAAGGGCAGTGTGTCGTAACGGGCAACTCTGC
AGCGGAACCGACTACTTTGGGTGTCCGTGTTTCTTTTTATTCTTGTATTGGCTGCTTATGGTG
ACAATTAAAGAATTGTTACCATATAGCTATTGGATTGGCCATCCAGTGTCAAACAGAGCTAT
TGTATATCTCTTTGTTGGATTCACACCTCTCACTCTTGAAACGTTACACACCCTCAATTACAT
TATACTGCTGAACACGAAGCGATGAATCCTAACCAGAAGATCATCACCATCGGCAGCGTGA
GCCTGACCATCAGCACCATCTGCTTCTTCATGCAGATCGCCATCCTGATCACCACCGTGACC
CTGCACTTCAAGCAGTACGAGTTCAACAGCCCTCCCAACAACCAGGTGATGCTGTGCGAGC
CCACCATCATCGAGAGGAACATGACCGAGATCGTGTACCTGACCAACACCACCATCGAGAA
GGAGATCTGCCCCAAGCCCGCCGAGTACAGGAACTGGAGCAAGCCCCAGTGCGGCATCACC
GGCTTCGCCCCTTTCAGCAAGGACAACAGCATCAGGCTGAGCGCCGGCGGCGACATCTGGG
TGACCAGGGAGCCCTACGTGAGCTGCGACCTGGACAAGTGCTACCAGTTCGCCCTGGGCCA
GGGCACCACCCTGAACAACGTGCACAGCAACAACACCGTGAGGGACAGGACCCCTTACCGG
ACCCTGCTGATGAACGAGCTGGGCGTGCCCTTCCACCTGGGCACCAAGCAGGTGTGCATCG
CCTGGAGCAGCTCCAGCTGCCACGACGGCAAGGCCTGGCTGCACGTGTGCATCACCGGCGA
CGACAAGAACGCCACCGCCAGCTTCATCTACAACGGCAGGCTGGTGGACAGCGTGGTGAGC
TGGAGCAACGACATCCTGAGGACCCAGGAGAGCGAGTGCGTGTGCATCAACGGCACCTGCA
CCGTGGTGATGACCGACGGCAACGCCACCGGCAAGGCCGACACCAAGATCCTGTTCATCGA
GGAGGGCAAGATCGTGCACACCAGCAAGCTGAGCGGCAGCGCCCAGCACGTGGAGGAGTG
CAGCTGCTACCCCAGGTACCCCGGCGTGAGGTGCGTGTGCAGGGACAACTGGAAGGGCAGC
AACCGGCCCATCATCGACATCAACATCAAGGACCACAGCATCGTCAGCAGGTACGTGTGCA
GCGGCCTGGTGGGCGACACCCCTAGAAAGAGCGACAGCAGCTCCAGCAGCCACTGCCTGAA
CCCCAACAACGAGAAGGGCGACCACGGCGTGAAGGGCTGGGCCTTCGACGACGGCAACGA
CGTGTGGATGGGCAGGACCATCAACGAGACAAGCAGGCTGGGCTACGAGACATTCAAGGTG
GTGGAGGGCTGGAGCAACCCCAAGAGCAAGCTGCAGATCAACAGGCAGGTGATCGTGGAC
AGGGGCGACAGGAGCGGCTACAGCGGCATCTTCAGCGTGGAGGGCAAGAGCTGCATCAAC
CGGTGCTTCTACGTGGAGCTGATCAGGGGCAGGAAGGAGGAGACAGAGGTGCTGTGGACCA
GCAACAGCATCGTGGTGTTCTGCGGCACCAGCGGCACCTACGGCACCGGCAGCTGGCCCGA
CGGCGCCAACCTGAGCCTGATGCACATCTGATAGTAAACTCGAAGCAGTGTTAAATCATTCA
GCTACCTGAGAGGGGCCCCTATAACTCTCTACGGCTAACCTGAATGGACTACGACATAGTCT
AGTCCGCCAAGGCCGAATTCTTAAAACAGCCTGTGGGTTGATCCCACCCACAGGCCCATTGG
GCGCTAGCACTCTGGTATCACGGTACCTTTGTGCGCCTGTTTTATACCCCCTCCCCCAACTGT
AACTTAGAAGTAACACACACCGATCAACAGTCAGCGTGGCACACCAGCCACGTTTTGATCA
AGCACTTCTGTTACCCCGGACTGAGTATCAATAGACTGCTCACGCGGTTGAAGGAGAAAGC
GTTCGTTATCCGGCCAACTACTTCGAAAAACCTAGTAACACCGTGGAAGTTGCAGAGTGTTT
CGCTCAGCACTACCCCAGTGTAGATCAGGTCGATGAGTCACCGCATTCCCCACGGGCGACC
GTGGCGGTGGCTGCGTTGGCGGCCTGCCCATGGGGAAACCCATGGGACGCTCTAATACAGA
CATGGTGCGAAGAGTCTATTGAGCTAGTTGGTAGTCCTCCGGCCCCTGAATGCGGCTAATCC
TAACTGCGGAGCACACACCCTCAAGCCAGAGGGCAGTGTGTCGTAACGGGCAACTCTGCAG
CGGAACCGACTACTTTGGGTGTCCGTGTTTCATTTTATTCCTATACTGGCTGCTTATGGTGAC
AATTGAGAGATCGTTACCATATAGCTATTGGATTGGCCATCCGGTGACTAATAGAGCTATTA
TATATCCCTTTGTTGGGTTTATACCACTTAGCTTGAAAGAGGTTAAAACATTACAATTCATTG
TTAAGTTGAATACAGCAAAATGAAGACCATCATCGCCCTGAGCTACATCCTGTGCCTGGTGT
TCGCCCAGAAGATCCCCGGCAATGACAACAGCACCGCCACCCTGTGCCTGGGCCACCACGC
CGTGCCCAACGGCACCATCGTGAAGACCATCACCAACGACAGGATCGAGGTGACCAACGCC
ACCGAGCTGGTGCAGAACAGCAGCATCGGCGAGATCTGCGACAGCCCTCACCAGATCCTGG
ACGGCGGCAACTGCACCCTGATCGACGCCCTGCTGGGCGACCCTCAGTGCGACGGCTTCCA
GAACAAGGAGTGGGACCTGTTCGTGGAGAGGAGCAGGGCCAACAGCAACTGCTACCCCTAC
GACGTGCCCGACTACGCCAGCCTGAGGAGCCTGGTGGCCAGCAGCGGCACCCTGGAGTTCA
AGAACGAGAGCTTCAACTGGACCGGCGTGAAGCAGAACGGCACCAGCAGCGCCTGCATCA
GGGGCAGCAGCTCCAGCTTCTTCAGCCGGCTGAATTGGCTCACCCACCTGAACTACACCTAC
CCCGCCCTGAACGTGACCATGCCCAACAACGAGCAGTTCGACAAGCTGTATATCTGGGGCG
TGCACCACCCCAGCACCGACAAGGACCAGATCAGCCTGTTCGCCCAGCCCAGCGGCAGGAT
CACCGTGAGCACCAAGAGGAGCCAGCAGGCCGTGATCCCCAACATCGGCAGCAGGCCCAG
GATCAGGGACATCCCCAGCAGGATCAGCATCTACTGGACCATCGTGAAGCCCGGCGACATC
CTGCTGATCAACAGCACCGGCAACCTGATCGCCCCTAGGGGCTACTTCAAGATCAGGAGCG
GCAAGAGCAGCATCATGAGGAGCGACGCCCCTATCGGCAAGTGCAAGAGCGAGTGCATCAC
CCCTAACGGCAGCATCCCCAACGACAAGCCCTTCCAGAACGTGAACAGGATCACCTACGGC
GCCTGCCCCAGGTACGTGAAGCAGAGCACCCTGAAGCTGGCCACCGGCATGAGGAACGTGC
CCGAGAAGCAGACCAGGGGCATCTTCGGCGCCATCGCCGGCTTCATCGAGAACGGCTGGGA
GGGCATGGTGGACGGCTGGTACGGCTTCAGGCACCAGAACAGCGAGGGCAGGGGCCAGGC
CGCCGACCTGAAGTCCACCCAGGCCGCCATCGACCAGATCAACGGCAAGCTGAACAGGCTG
ATCGGCAAGACCAACGAGAAGTTCCACCAGATCGAGAAGGAGTTCAGCGAGGTGGAGGGC
AGGGTGCAGGACCTGGAGAAGTACGTGGAGGACACCAAGATCGACCTGTGGAGCTACAAC
GCCGAGCTGCTGGTGGCCCTGGAGAACCAGCACACCATCGACCTGACCGACAGCGAGATGA
ACAAGCTGTTCGAGAAGACCAAGAAGCAGCTGAGGGAGAACGCCGAGGACATGGGCAACG
GCTGCTTCAAGATCTACCACAAGTGCGACAACGCCTGCATCGGCAGCATCAGGAACGAGAC
ATATGACCACAACGTGTACCGGGACGAGGCCCTGAACAACCGGTTCCAGATCAAGGGCGTG
GAGCTGAAGTCCGGCTACAAGGACTGGATCCTGTGGATCAGCTTCGCCATGAGCTGCTTCCT
GCTGTGCATCGCCCTGCTGGGCTTCATCATGTGGGCCTGCCAGAAGGGCAACATCAGGTGCA
ACATCTGCATCTGATAGTAAACTCGAGTATGTTACGTGCAAAGGTGATTGTCACCCCCCGAA
AGACCATATTGTGACACACCCTCAGTATCACGCCCAAACATTTACAGCCGCGGTGTCAAAA
ACCGCGTGGACGTGGTTAACATCCCTGCTGGGAGGATCAGCCGTAATTATTATAATTGGCTT
GGTGCTGGCTACTATTGTGGCCATGTACGTGCTGACCAACCAGAAACATAATTGAATACAGC
AGCAATTGGCAAGCTGCTTACATAGAACTCGCGGCGATTGGCATGCCGCCTTAAAATTTTTA
TTTTATTTTTTCTTTTCTTTTCCGAATCGGATTTTGTTTTTAATATTTC
SEQ ID NO: 157 (sgP EV71 NA sgP EMCV HA)
ATGGGCGGCGCATGAGAGAAGCCCAGACCAATTACCTACCCAAAATGGAGAAAGTTCACGT
TGACATCGAGGAAGACAGCCCATTCCTCAGAGCTTTGCAGCGGAGCTTCCCGCAGTTTGAG
GTAGAAGCCAAGCAGGTCACTGATAATGACCATGCTAATGCCAGAGCGTTTTCGCATCTGG
CTTCAAAACTGATCGAAACGGAGGTGGACCCATCCGACACGATCCTTGACATTGGAAGTGC
GCCCGCCCGCAGAATGTATTCTAAGCACAAGTATCATTGTATCTGTCCGATGAGATGTGCGG
AAGATCCGGACAGATTGTATAAGTATGCAACTAAGCTGAAGAAAAACTGTAAGGAAATAAC
TGATAAGGAATTGGACAAGAAAATGAAGGAGCTGGCCGCCGTCATGAGCGACCCTGACCTG
GAAACTGAGACTATGTGCCTCCACGACGACGAGTCGTGTCGCTACGAAGGGCAAGTCGCTG
TTTACCAGGATGTATACGCCGTCGACGGCCCCACCAGCCTGTACCACCAGGCCAACAAGGG
CGTGAGGGTGGCCTACTGGATCGGCTTCGACACCACACCCTTCATGTTCAAGAACCTGGCCG
GCGCCTACCCCAGCTACAGCACCAACTGGGCCGACGAGACAGTGCTGACCGCCAGGAACAT
CGGCCTGTGCAGCAGCGACGTGATGGAGAGGAGCCGGAGGGGCATGAGCATCCTGAGGAA
GAAGTACCTGAAGCCCAGCAACAACGTGCTGTTCAGCGTGGGCAGCACCATCTACCACGAG
AAGAGGGACCTGCTGAGGAGCTGGCACCTGCCCAGCGTGTTCCACCTGAGGGGCAAGCAGA
ACTACACCTGCAGGTGCGAGACAATCGTGAGCTGCGACGGCTACGTGGTGAAGAGGATCGC
CATCAGCCCCGGCCTGTACGGCAAGCCCAGCGGCTACGCCGCCACCATGCACAGGGAGGGC
TTCCTGTGCTGCAAGGTGACCGACACCCTGAACGGCGAGAGGGTGAGCTTCCCCGTGTGCA
CCTACGTGCCCGCCACCCTGTGCGACCAGATGACCGGCATCCTGGCCACCGACGTGAGCGC
CGACGACGCCCAGAAGCTGCTGGTGGGCCTGAACCAGAGGATCGTGGTGAACGGCAGGACC
CAGAGGAACACCAACACCATGAAGAACTACCTGCTGCCCGTGGTGGCCCAGGCCTTCGCCA
GGTGGGCCAAGGAGTACAAGGAGGACCAGGAGGACGAGAGGCCCCTGGGCCTGAGGGACC
GACAGCTGGTGATGGGCTGCTGCTGGGCCTTCAGGCGGCACAAGATCACCAGCATCTACAA
GAGGCCCGACACCCAGACCATCATCAAGGTGAACAGCGACTTCCACAGCTTCGTGCTGCCC
AGGATCGGCAGCAACACCCTGGAGATCGGCCTGAGGACCCGGATCAGGAAGATGCTGGAG
GAGCACAAGGAGCCCAGCCCTCTGATCACCGCCGAGGACGTGCAGGAGGCCAAGTGCGCC
GCCGACGAGGCCAAGGAGGTGAGGGAGGCCGAGGAGCTGAGGGCCGCCCTGCCTCCCCTG
GCCGCCGACGTGGAGGAGCCCACCCTGGAGGCCGACGTGGACCTGATGCTGCAGGAGGCCG
GCGCCGGCAGCGTGGAGACACCCAGGGGCCTGATCAAGGTGACCAGCTACGACGGCGAGG
ACAAGATCGGCAGCTACGCCGTGCTCAGCCCTCAGGCCGTGCTGAAGTCCGAGAAGCTGAG
CTGCATCCACCCTCTGGCCGAGCAGGTGATCGTGATCACCCACAGCGGCAGGAAGGGCAGG
TACGCCGTGGAGCCCTACCACGGCAAGGTGGTGGTCCCCGAGGGCCACGCCATCCCCGTGC
AGGACTTCCAGGCCCTGAGCGAGAGCGCCACCATCGTGTATAACGAGAGGGAGTTCGTGAA
CAGGTACCTGCACCACATCGCCACCCACGGCGGCGCCCTGAACACCGACGAGGAGTACTAC
AAGACCGTGAAGCCCAGCGAGCACGACGGCGAGTACCTGTACGACATCGACAGGAAGCAG
TGCGTGAAGAAGGAGCTGGTGACCGGCCTGGGCCTGACCGGCGAGCTGGTGGACCCTCCCT
TCCACGAGTTCGCCTACGAGAGCCTGAGGACCAGGCCCGCCGCTCCCTACCAGGTGCCCAC
CATCGGCGTGTACGGCGTGCCCGGCAGCGGCAAGAGCGGCATCATCAAGAGCGCCGTGACC
AAGAAGGACCTGGTGGTGAGCGCCAAGAAGGAGAACTGCGCCGAGATCATCAGGGACGTG
AAGAAGATGAAGGGCCTGGACGTGAACGCCAGGACCGTGGACAGCGTGCTCCTGAACGGCT
GCAAGCACCCCGTGGAGACACTGTATATCGACGAGGCCTTCGCCTGCCACGCCGGCACCCT
GAGGGCCCTGATCGCCATCATCAGGCCCAAGAAGGCCGTGCTGTGCGGCGACCCCAAGCAG
TGCGGCTTCTTCAACATGATGTGCCTGAAGGTGCACTTCAACCACGAGATCTGCACCCAGGT
GTTCCACAAGAGCATCAGCAGGCGGTGCACCAAGAGCGTGACCAGCGTGGTGAGCACCCTG
TTCTACGACAAGAAGATGAGGACCACCAACCCCAAGGAGACAAAGATCGTGATCGACACCA
CCGGCAGCACCAAGCCCAAGCAGGACGACCTGATCCTGACCTGCTTCAGGGGCTGGGTGAA
GCAGCTGCAGATCGACTACAAGGGCAACGAGATCATGACCGCCGCCGCTAGCCAGGGCCTG
ACCAGGAAGGGCGTGTACGCCGTGAGGTACAAGGTGAACGAGAATCCCCTGTACGCCCCTA
CCAGCGAGCACGTGAACGTCCTGCTGACCAGGACCGAGGACAGGATCGTGTGGAAGACCCT
GGCCGGCGACCCCTGGATCAAGACCCTGACCGCCAAGTACCCCGGCAACTTCACCGCCACC
ATCGAGGAGTGGCAGGCCGAGCACGACGCCATCATGAGGCACATCCTGGAGAGGCCCGACC
CCACCGACGTGTTCCAGAACAAGGCCAACGTGTGCTGGGCCAAGGCCCTGGTGCCCGTGCT
GAAGACCGCCGGCATCGACATGACCACCGAGCAGTGGAACACCGTGGACTACTTCGAGACA
GACAAGGCCCACAGCGCCGAGATCGTGCTGAACCAGCTGTGCGTGAGGTTCTTCGGCCTGG
ACCTGGACAGCGGCCTGTTCAGCGCCCCTACCGTGCCCCTGAGCATCAGGAACAACCACTG
GGACAACAGCCCCAGCCCCAACATGTACGGCCTGAACAAGGAGGTGGTGAGGCAGCTGAG
CAGGCGGTACCCTCAGCTGCCCAGGGCCGTGGCCACCGGCAGGGTGTACGACATGAACACC
GGCACCCTGAGGAACTACGACCCCAGGATCAACCTGGTGCCCGTGAACAGGCGGCTGCCAC
ACGCCCTGGTGCTGCACCACAACGAGCACCCTCAGAGCGACTTCAGCAGCTTCGTGAGCAA
GCTGAAGGGCAGGACCGTGCTGGTGGTGGGCGAGAAGCTGAGCGTGCCCGGCAAGATGGT
GGACTGGCTGAGCGACAGGCCCGAGGCCACCTTCCGGGCCAGGCTGGACCTGGGCATCCCC
GGCGACGTGCCCAAGTACGACATCATCTTCGTGAACGTGAGGACCCCTTACAAGTACCACC
ACTACCAGCAGTGCGAGGACCACGCCATCAAGCTGAGCATGCTGACCAAGAAGGCCTGCCT
GCACCTGAACCCCGGCGGCACCTGCGTGAGCATCGGCTACGGCTACGCCGACAGGGCCAGC
GAGAGCATCATCGGCGCCATCGCCAGGCTGTTCAAGTTCAGCAGGGTGTGCAAGCCCAAGA
GCAGCCTGGAGGAGACAGAGGTGCTGTTCGTGTTCATCGGCTACGACCGGAAGGCCAGGAC
CCACAACCCCTACAAGCTGAGCAGCACCCTGACCAACATCTACACCGGCAGCAGGCTGCAC
GAGGCCGGCTGCGCCCCTAGCTACCACGTGGTGAGGGGCGACATCGCCACCGCCACCGAGG
GCGTGATCATCAACGCCGCCAACAGCAAGGGCCAGCCCGGCGGCGGGGTGTGCGGCGCCCT
GTATAAGAAGTTCCCCGAGAGCTTCGACCTGCAGCCCATCGAGGTGGGCAAGGCCAGGCTG
GTGAAGGGCGCCGCCAAGCACATCATCCACGCCGTGGGCCCCAACTTCAACAAGGTGAGCG
AGGTGGAGGGCGACAAGCAGCTGGCCGAGGCCTACGAGAGCATCGCCAAGATCGTGAACG
ACAACAACTACAAGAGCGTGGCCATCCCTCTGCTGAGCACCGGCATCTTCAGCGGCAACAA
GGACAGGCTGACCCAGAGCCTGAACCACCTGCTGACCGCCCTGGACACCACCGACGCCGAC
GTGGCCATCTACTGCAGGGACAAGAAGTGGGAGATGACCCTGAAGGAGGCCGTGGCCAGG
CGGGAGGCCGTGGAGGAGATCTGCATCAGCGACGACAGCAGCGTGACGGAGCCCGACGCC
GAGCTGGTGAGGGTGCACCCCAAGAGCAGCCTGGCCGGCAGGAAGGGCTACAGCACCAGC
GACGGCAAGACCTTCAGCTACCTGGAGGGCACCAAGTTCCACCAGGCCGCCAAGGACATCG
CCGAGATCAACGCCATGTGGCCCGTGGCCACCGAGGCCAACGAGCAGGTGTGCATGTATAT
CCTGGGCGAGAGCATGAGCAGCATCAGGAGCAAGTGCCCCGTGGAGGAGAGCGAGGCCAG
CACCCCTCCCAGCACCCTGCCCTGCCTGTGCATCCACGCCATGACCCCTGAGAGGGTGCAGC
GGCTGAAGGCCAGCAGGCCCGAGCAGATCACCGTGTGCAGCAGCTTCCCTCTGCCCAAGTA
CCGGATCACCGGCGTGCAGAAGATCCAGTGCAGCCAGCCCATCCTGTTCAGCCCCAAGGTG
CCCGCCTACATCCACCCCAGGAAGTACCTGGTGGAGACACCCCCCGTGGACGAGACACCCG
AGCCCAGCGCCGAGAACCAGAGCACCGAGGGCACCCCTGAGCAGCCTCCCCTGATCACCGA
GGACGAGACAAGGACCAGGACGCCCGAGCCCATCATCATTGAGGAGGAAGAGGAGGACAG
CATCAGCCTGCTGAGCGACGGCCCCACCCACCAGGTGCTGCAGGTGGAGGCCGACATCCAC
GGCCCTCCCAGCGTGAGCAGCTCCAGCTGGAGCATCCCTCACGCCAGCGACTTCGACGTGG
ACAGCCTGAGCATCCTGGACACCCTGGAGGGCGCCAGCGTGACCAGCGGCGCCACCAGCGC
CGAGACAAACAGCTACTTCGCCAAGAGCATGGAGTTCCTGGCCAGGCCCGTGCCCGCCCCT
AGGACCGTGTTCAGGAACCCTCCCCACCCCGCCCCTAGGACCAGGACCCCTAGCCTGGCCC
CTAGCAGGGCCTGCAGCAGGACCAGCCTGGTGAGCACCCCTCCCGGCGTGAACCGGGTGAT
CACCAGGGAGGAGCTGGAGGCCCTGACCCCTAGCAGGACCCCTAGCAGGAGCGTGAGCAG
GACCAGCCTGGTGAGCAACCCTCCCGGCGTGAACCGGGTGATCACCAGGGAGGAGTTCGAG
GCCTTCGTGGCCCAGCAGCAAAGGCGGTTCGACGCCGGCGCCTACATCTTCAGCAGCGACA
CCGGCCAGGGCCACCTGCAGCAGAAGTCCGTGAGGCAGACCGTGCTGAGCGAGGTGGTCCT
GGAGAGGACGGAGCTGGAGATCAGCTACGCCCCTAGGCTGGACCAGGAGAAGGAGGAGCT
GCTGAGGAAGAAGCTGCAGCTGAACCCCACCCCTGCCAACAGGAGCAGGTACCAGAGCAG
GAAGGTGGAGAACATGAAGGCCATCACCGCCAGGCGGATCCTGCAGGGCCTGGGCCACTAC
CTGAAGGCCGAGGGCAAGGTGGAGTGCTACAGGACCCTGCACCCCGTGCCCCTGTACTCCA
GCTCCGTGAACAGGGCCTTCAGCAGCCCCAAGGTGGCCGTGGAGGCCTGCAACGCCATGCT
GAAGGAGAACTTCCCCACCGTGGCCAGCTACTGCATCATCCCCGAGTACGACGCCTACCTG
GACATGGTGGACGGCGCCAGCTGCTGCCTGGACACCGCCAGCTTCTGCCCCGCCAAGCTGA
GGAGCTTCCCCAAGAAGCACAGCTACCTGGAGCCCACCATCAGGAGCGCCGTGCCCAGCGC
CATCCAGAACACCCTGCAGAACGTGCTGGCCGCCGCTACCAAGAGGAACTGCAACGTGACC
CAGATGAGGGAGCTGCCCGTGCTGGACAGCGCCGCCTTCAACGTGGAGTGCTTCAAGAAGT
ACGCCTGCAACAACGAGTACTGGGAGACATTCAAGGAGAACCCCATCAGGCTGACCGAGGA
GAACGTGGTGAACTACATCACCAAGCTGAAGGGCCCCAAGGCCGCCGCTCTGTTCGCCAAG
ACCCACAACCTGAACATGCTCCAGGACATCCCTATGGACAGGTTCGTGATGGACCTGAAGA
GGGACGTGAAGGTGACCCCTGGCACCAAGCACACCGAGGAGAGGCCCAAGGTGCAGGTGA
TCCAGGCCGCCGACCCTCTGGCCACCGCCTACCTGTGCGGCATCCACAGGGAGCTGGTGAG
GCGGCTGAACGCCGTCCTGCTGCCCAACATCCACACCCTGTTCGACATGAGCGCCGAGGAC
TTCGACGCCATCATCGCCGAGCACTTCCAGCCCGGCGACTGCGTGCTGGAGACAGACATCG
CCAGCTTCGACAAGAGCGAGGACGACGCTATGGCCCTGACCGCCCTGATGATCCTGGAGGA
CCTGGGCGTGGACGCCGAGCTGCTGACCCTGATCGAGGCCGCCTTCGGCGAGATCAGCAGC
ATCCACCTGCCCACCAAGACCAAGTTCAAGTTCGGCGCCATGATGAAGTCCGGCATGTTCCT
GACCCTGTTCGTGAACACCGTGATCAACATCGTGATCGCCAGCAGGGTGCTGCGGGAGAGG
CTGACCGGCAGCCCCTGCGCCGCCTTCATCGGCGACGACAACATCGTGAAGGGCGTGAAGT
CCGACAAGCTGATGGCCGACAGGTGCGCCACCTGGCTGAACATGGAGGTGAAGATCATCGA
CGCCGTGGTGGGCGAGAAGGCCCCTTACTTCTGCGGCGGCTTCATCCTGTGCGACAGCGTGA
CCGGCACCGCCTGCAGGGTGGCCGACCCTCTGAAGAGGCTGTTCAAGCTGGGCAAGCCCCT
GGCCGCCGACGACGAGCACGACGACGATAGGCGGAGGGCCCTGCACGAGGAGAGCACCAG
GTGGAACCGGGTGGGCATCCTGAGCGAGCTGTGCAAGGCCGTGGAGAGCAGGTACGAGAC
AGTGGGCACCAGCATCATCGTGATGGCCATGACCACCCTGGCCAGCAGCGTCAAGTCCTTC
AGCTACCTGAGGGGGGCCCCTATAACTCTCTACGGCTAACCTGAATGGACTACGACATAGTC
TAGTCCGCCAAGGCCCTTAAGTTAAAACAGCTGTGGGTTGTCACCCACCCACAGGGTCCACT
GGGCGCTAGTACACTGGTATCTCGGTACCTTTGTACGCCTGTTTTATACCCCCTCCCTGATTT
GCAACTTAGAAGCAACGCAAACCAGATCAATAGTAGGTGTGACATACCAGTCGCATCTTGA
TCAAGCACTTCTGTATCCCCGGACCGAGTATCAATAGACTGTGCACACGGTTGAAGGAGAA
AACGTCCGTTACCCGGCTAACTACTTCGAGAAGCCTAGTAACGCCATTGAAGTTGCAGAGTG
TTTCGCTCAGCACTCCCCCCGTGTAGATCAGGTCGATGAGTCACCGCATTCCCCACGGGCGA
CCGTGGCGGTGGCTGCGTTGGCGGCCTGCCTATGGGGTAACCCATAGGACGCTCTAATACG
GACATGGCGTGAAGAGTCTATTGAGCTAGTTAGTAGTCCTCCGGCCCCTGAATGCGGCTAAT
CCTAACTGCGGAGCACATACCCTTAATCCAAAGGGCAGTGTGTCGTAACGGGCAACTCTGC
AGCGGAACCGACTACTTTGGGTGTCCGTGTTTCTTTTTATTCTTGTATTGGCTGCTTATGGTG
ACAATTAAAGAATTGTTACCATATAGCTATTGGATTGGCCATCCAGTGTCAAACAGAGCTAT
TGTATATCTCTTTGTTGGATTCACACCTCTCACTCTTGAAACGTTACACACCCTCAATTACAT
TATACTGCTGAACACGAAGCGATGAATCCTAACCAGAAGATCATCACCATCGGCAGCGTGA
GCCTGACCATCAGCACCATCTGCTTCTTCATGCAGATCGCCATCCTGATCACCACCGTGACC
CTGCACTTCAAGCAGTACGAGTTCAACAGCCCTCCCAACAACCAGGTGATGCTGTGCGAGC
CCACCATCATCGAGAGGAACATGACCGAGATCGTGTACCTGACCAACACCACCATCGAGAA
GGAGATCTGCCCCAAGCCCGCCGAGTACAGGAACTGGAGCAAGCCCCAGTGCGGCATCACC
GGCTTCGCCCCTTTCAGCAAGGACAACAGCATCAGGCTGAGCGCCGGCGGCGACATCTGGG
TGACCAGGGAGCCCTACGTGAGCTGCGACCTGGACAAGTGCTACCAGTTCGCCCTGGGCCA
GGGCACCACCCTGAACAACGTGCACAGCAACAACACCGTGAGGGACAGGACCCCTTACCGG
ACCCTGCTGATGAACGAGCTGGGCGTGCCCTTCCACCTGGGCACCAAGCAGGTGTGCATCG
CCTGGAGCAGCTCCAGCTGCCACGACGGCAAGGCCTGGCTGCACGTGTGCATCACCGGCGA
CGACAAGAACGCCACCGCCAGCTTCATCTACAACGGCAGGCTGGTGGACAGCGTGGTGAGC
TGGAGCAACGACATCCTGAGGACCCAGGAGAGCGAGTGCGTGTGCATCAACGGCACCTGCA
CCGTGGTGATGACCGACGGCAACGCCACCGGCAAGGCCGACACCAAGATCCTGTTCATCGA
GGAGGGCAAGATCGTGCACACCAGCAAGCTGAGCGGCAGCGCCCAGCACGTGGAGGAGTG
CAGCTGCTACCCCAGGTACCCCGGCGTGAGGTGCGTGTGCAGGGACAACTGGAAGGGCAGC
AACCGGCCCATCATCGACATCAACATCAAGGACCACAGCATCGTCAGCAGGTACGTGTGCA
GCGGCCTGGTGGGCGACACCCCTAGAAAGAGCGACAGCAGCTCCAGCAGCCACTGCCTGAA
CCCCAACAACGAGAAGGGCGACCACGGCGTGAAGGGCTGGGCCTTCGACGACGGCAACGA
CGTGTGGATGGGCAGGACCATCAACGAGACAAGCAGGCTGGGCTACGAGACATTCAAGGTG
GTGGAGGGCTGGAGCAACCCCAAGAGCAAGCTGCAGATCAACAGGCAGGTGATCGTGGAC
AGGGGCGACAGGAGCGGCTACAGCGGCATCTTCAGCGTGGAGGGCAAGAGCTGCATCAAC
CGGTGCTTCTACGTGGAGCTGATCAGGGGCAGGAAGGAGGAGACAGAGGTGCTGTGGACCA
GCAACAGCATCGTGGTGTTCTGCGGCACCAGCGGCACCTACGGCACCGGCAGCTGGCCCGA
CGGCGCCAACCTGAGCCTGATGCACATCTGATAGTAAACTCGAAGCAGTGTTAAATCATTCA
GCTACCTGAGAGGGGCCCCTATAACTCTCTACGGCTAACCTGAATGGACTACGACATAGTCT
AGTCCGCCAAGGCCGAATTCCTCCCTCCCCCCCCCCTAACGTTACTGGCCGAAGCCGCTTGG
AATAAGGCCGGTGTGCGTTTGTCTATATGTTATTTTCCACCATATTGCCGTCTTTTGGCAATG
TGAGGGCCCGGAAACCTGGCCCTGTCTTCTTGACGAGCATTCCTAGGGGTCTTTCCCCTCTC
GCCAAAGGAATGCAAGGTCTGTTGAATGTCGTGAAGGAAGCAGTTCCTCTGGAAGCTTCTT
GAAGACAAACAACGTCTGTAGCGACCCTTTGCAGGCAGCGGAACCCCCCACCTGGCGACAG
GTGCCTCTGCGGCCAAAAGCCACGTGTATAAGATACACCTGCAAAGGCGGCACAACCCCAG
TGCCACGTTGTGAGTTGGATAGTTGTGGAAAGAGTCAAATGGCTCTCCTCAAGCGTATTCAA
CAAGGGGCTGAAGGATGCCCAGAAGGTACCCCATTGTATGGGATCTGATCTGGGGCCTCGG
TGCACATGCTTTACGTGTGTTTAGTCGAGGTTAAAAAACGTCTAGGCCCCCCGAACCACGGG
GACGTGGTTTTCCTTTGAAAAACACGATGATAATATGAAGACCATCATCGCCCTGAGCTACA
TCCTGTGCCTGGTGTTCGCCCAGAAGATCCCCGGCAATGACAACAGCACCGCCACCCTGTGC
CTGGGCCACCACGCCGTGCCCAACGGCACCATCGTGAAGACCATCACCAACGACAGGATCG
AGGTGACCAACGCCACCGAGCTGGTGCAGAACAGCAGCATCGGCGAGATCTGCGACAGCCC
TCACCAGATCCTGGACGGCGGCAACTGCACCCTGATCGACGCCCTGCTGGGCGACCCTCAG
TGCGACGGCTTCCAGAACAAGGAGTGGGACCTGTTCGTGGAGAGGAGCAGGGCCAACAGC
AACTGCTACCCCTACGACGTGCCCGACTACGCCAGCCTGAGGAGCCTGGTGGCCAGCAGCG
GCACCCTGGAGTTCAAGAACGAGAGCTTCAACTGGACCGGCGTGAAGCAGAACGGCACCAG
CAGCGCCTGCATCAGGGGCAGCAGCTCCAGCTTCTTCAGCCGGCTGAATTGGCTCACCCACC
TGAACTACACCTACCCCGCCCTGAACGTGACCATGCCCAACAACGAGCAGTTCGACAAGCT
GTATATCTGGGGCGTGCACCACCCCAGCACCGACAAGGACCAGATCAGCCTGTTCGCCCAG
CCCAGCGGCAGGATCACCGTGAGCACCAAGAGGAGCCAGCAGGCCGTGATCCCCAACATCG
GCAGCAGGCCCAGGATCAGGGACATCCCCAGCAGGATCAGCATCTACTGGACCATCGTGAA
GCCCGGCGACATCCTGCTGATCAACAGCACCGGCAACCTGATCGCCCCTAGGGGCTACTTC
AAGATCAGGAGCGGCAAGAGCAGCATCATGAGGAGCGACGCCCCTATCGGCAAGTGCAAG
AGCGAGTGCATCACCCCTAACGGCAGCATCCCCAACGACAAGCCCTTCCAGAACGTGAACA
GGATCACCTACGGCGCCTGCCCCAGGTACGTGAAGCAGAGCACCCTGAAGCTGGCCACCGG
CATGAGGAACGTGCCCGAGAAGCAGACCAGGGGCATCTTCGGCGCCATCGCCGGCTTCATC
GAGAACGGCTGGGAGGGCATGGTGGACGGCTGGTACGGCTTCAGGCACCAGAACAGCGAG
GGCAGGGGCCAGGCCGCCGACCTGAAGTCCACCCAGGCCGCCATCGACCAGATCAACGGCA
AGCTGAACAGGCTGATCGGCAAGACCAACGAGAAGTTCCACCAGATCGAGAAGGAGTTCA
GCGAGGTGGAGGGCAGGGTGCAGGACCTGGAGAAGTACGTGGAGGACACCAAGATCGACC
TGTGGAGCTACAACGCCGAGCTGCTGGTGGCCCTGGAGAACCAGCACACCATCGACCTGAC
CGACAGCGAGATGAACAAGCTGTTCGAGAAGACCAAGAAGCAGCTGAGGGAGAACGCCGA
GGACATGGGCAACGGCTGCTTCAAGATCTACCACAAGTGCGACAACGCCTGCATCGGCAGC
ATCAGGAACGAGACATATGACCACAACGTGTACCGGGACGAGGCCCTGAACAACCGGTTCC
AGATCAAGGGCGTGGAGCTGAAGTCCGGCTACAAGGACTGGATCCTGTGGATCAGCTTCGC
CATGAGCTGCTTCCTGCTGTGCATCGCCCTGCTGGGCTTCATCATGTGGGCCTGCCAGAAGG
GCAACATCAGGTGCAACATCTGCATCTGATAGTAAACTCGAGTATGTTACGTGCAAAGGTG
ATTGTCACCCCCCGAAAGACCATATTGTGACACACCCTCAGTATCACGCCCAAACATTTACA
GCCGCGGTGTCAAAAACCGCGTGGACGTGGTTAACATCCCTGCTGGGAGGATCAGCCGTAA
TTATTATAATTGGCTTGGTGCTGGCTACTATTGTGGCCATGTACGTGCTGACCAACCAGAAA
CATAATTGAATACAGCAGCAATTGGCAAGCTGCTTACATAGAACTCGCGGCGATTGGCATG
CCGCCTTAAAATTTTTATTTTATTTTTTCTTTTCTTTTCCGAATCGGATTTTGTTTTTAATATTT
C
SEQ ID NO: 158 (sgP EV71 NA sgP EV71 HA)
ATGGGCGGCGCATGAGAGAAGCCCAGACCAATTACCTACCCAAAATGGAGAAAGTTCACGT
TGACATCGAGGAAGACAGCCCATTCCTCAGAGCTTTGCAGCGGAGCTTCCCGCAGTTTGAG
GTAGAAGCCAAGCAGGTCACTGATAATGACCATGCTAATGCCAGAGCGTTTTCGCATCTGG
CTTCAAAACTGATCGAAACGGAGGTGGACCCATCCGACACGATCCTTGACATTGGAAGTGC
GCCCGCCCGCAGAATGTATTCTAAGCACAAGTATCATTGTATCTGTCCGATGAGATGTGCGG
AAGATCCGGACAGATTGTATAAGTATGCAACTAAGCTGAAGAAAAACTGTAAGGAAATAAC
TGATAAGGAATTGGACAAGAAAATGAAGGAGCTGGCCGCCGTCATGAGCGACCCTGACCTG
GAAACTGAGACTATGTGCCTCCACGACGACGAGTCGTGTCGCTACGAAGGGCAAGTCGCTG
TTTACCAGGATGTATACGCCGTCGACGGCCCCACCAGCCTGTACCACCAGGCCAACAAGGG
CGTGAGGGTGGCCTACTGGATCGGCTTCGACACCACACCCTTCATGTTCAAGAACCTGGCCG
GCGCCTACCCCAGCTACAGCACCAACTGGGCCGACGAGACAGTGCTGACCGCCAGGAACAT
CGGCCTGTGCAGCAGCGACGTGATGGAGAGGAGCCGGAGGGGCATGAGCATCCTGAGGAA
GAAGTACCTGAAGCCCAGCAACAACGTGCTGTTCAGCGTGGGCAGCACCATCTACCACGAG
AAGAGGGACCTGCTGAGGAGCTGGCACCTGCCCAGCGTGTTCCACCTGAGGGGCAAGCAGA
ACTACACCTGCAGGTGCGAGACAATCGTGAGCTGCGACGGCTACGTGGTGAAGAGGATCGC
CATCAGCCCCGGCCTGTACGGCAAGCCCAGCGGCTACGCCGCCACCATGCACAGGGAGGGC
TTCCTGTGCTGCAAGGTGACCGACACCCTGAACGGCGAGAGGGTGAGCTTCCCCGTGTGCA
CCTACGTGCCCGCCACCCTGTGCGACCAGATGACCGGCATCCTGGCCACCGACGTGAGCGC
CGACGACGCCCAGAAGCTGCTGGTGGGCCTGAACCAGAGGATCGTGGTGAACGGCAGGACC
CAGAGGAACACCAACACCATGAAGAACTACCTGCTGCCCGTGGTGGCCCAGGCCTTCGCCA
GGTGGGCCAAGGAGTACAAGGAGGACCAGGAGGACGAGAGGCCCCTGGGCCTGAGGGACC
GACAGCTGGTGATGGGCTGCTGCTGGGCCTTCAGGCGGCACAAGATCACCAGCATCTACAA
GAGGCCCGACACCCAGACCATCATCAAGGTGAACAGCGACTTCCACAGCTTCGTGCTGCCC
AGGATCGGCAGCAACACCCTGGAGATCGGCCTGAGGACCCGGATCAGGAAGATGCTGGAG
GAGCACAAGGAGCCCAGCCCTCTGATCACCGCCGAGGACGTGCAGGAGGCCAAGTGCGCC
GCCGACGAGGCCAAGGAGGTGAGGGAGGCCGAGGAGCTGAGGGCCGCCCTGCCTCCCCTG
GCCGCCGACGTGGAGGAGCCCACCCTGGAGGCCGACGTGGACCTGATGCTGCAGGAGGCCG
GCGCCGGCAGCGTGGAGACACCCAGGGGCCTGATCAAGGTGACCAGCTACGACGGCGAGG
ACAAGATCGGCAGCTACGCCGTGCTCAGCCCTCAGGCCGTGCTGAAGTCCGAGAAGCTGAG
CTGCATCCACCCTCTGGCCGAGCAGGTGATCGTGATCACCCACAGCGGCAGGAAGGGCAGG
TACGCCGTGGAGCCCTACCACGGCAAGGTGGTGGTCCCCGAGGGCCACGCCATCCCCGTGC
AGGACTTCCAGGCCCTGAGCGAGAGCGCCACCATCGTGTATAACGAGAGGGAGTTCGTGAA
CAGGTACCTGCACCACATCGCCACCCACGGCGGCGCCCTGAACACCGACGAGGAGTACTAC
AAGACCGTGAAGCCCAGCGAGCACGACGGCGAGTACCTGTACGACATCGACAGGAAGCAG
TGCGTGAAGAAGGAGCTGGTGACCGGCCTGGGCCTGACCGGCGAGCTGGTGGACCCTCCCT
TCCACGAGTTCGCCTACGAGAGCCTGAGGACCAGGCCCGCCGCTCCCTACCAGGTGCCCAC
CATCGGCGTGTACGGCGTGCCCGGCAGCGGCAAGAGCGGCATCATCAAGAGCGCCGTGACC
AAGAAGGACCTGGTGGTGAGCGCCAAGAAGGAGAACTGCGCCGAGATCATCAGGGACGTG
AAGAAGATGAAGGGCCTGGACGTGAACGCCAGGACCGTGGACAGCGTGCTCCTGAACGGCT
GCAAGCACCCCGTGGAGACACTGTATATCGACGAGGCCTTCGCCTGCCACGCCGGCACCCT
GAGGGCCCTGATCGCCATCATCAGGCCCAAGAAGGCCGTGCTGTGCGGCGACCCCAAGCAG
TGCGGCTTCTTCAACATGATGTGCCTGAAGGTGCACTTCAACCACGAGATCTGCACCCAGGT
GTTCCACAAGAGCATCAGCAGGCGGTGCACCAAGAGCGTGACCAGCGTGGTGAGCACCCTG
TTCTACGACAAGAAGATGAGGACCACCAACCCCAAGGAGACAAAGATCGTGATCGACACCA
CCGGCAGCACCAAGCCCAAGCAGGACGACCTGATCCTGACCTGCTTCAGGGGCTGGGTGAA
GCAGCTGCAGATCGACTACAAGGGCAACGAGATCATGACCGCCGCCGCTAGCCAGGGCCTG
ACCAGGAAGGGCGTGTACGCCGTGAGGTACAAGGTGAACGAGAATCCCCTGTACGCCCCTA
CCAGCGAGCACGTGAACGTCCTGCTGACCAGGACCGAGGACAGGATCGTGTGGAAGACCCT
GGCCGGCGACCCCTGGATCAAGACCCTGACCGCCAAGTACCCCGGCAACTTCACCGCCACC
ATCGAGGAGTGGCAGGCCGAGCACGACGCCATCATGAGGCACATCCTGGAGAGGCCCGACC
CCACCGACGTGTTCCAGAACAAGGCCAACGTGTGCTGGGCCAAGGCCCTGGTGCCCGTGCT
GAAGACCGCCGGCATCGACATGACCACCGAGCAGTGGAACACCGTGGACTACTTCGAGACA
GACAAGGCCCACAGCGCCGAGATCGTGCTGAACCAGCTGTGCGTGAGGTTCTTCGGCCTGG
ACCTGGACAGCGGCCTGTTCAGCGCCCCTACCGTGCCCCTGAGCATCAGGAACAACCACTG
GGACAACAGCCCCAGCCCCAACATGTACGGCCTGAACAAGGAGGTGGTGAGGCAGCTGAG
CAGGCGGTACCCTCAGCTGCCCAGGGCCGTGGCCACCGGCAGGGTGTACGACATGAACACC
GGCACCCTGAGGAACTACGACCCCAGGATCAACCTGGTGCCCGTGAACAGGCGGCTGCCAC
ACGCCCTGGTGCTGCACCACAACGAGCACCCTCAGAGCGACTTCAGCAGCTTCGTGAGCAA
GCTGAAGGGCAGGACCGTGCTGGTGGTGGGCGAGAAGCTGAGCGTGCCCGGCAAGATGGT
GGACTGGCTGAGCGACAGGCCCGAGGCCACCTTCCGGGCCAGGCTGGACCTGGGCATCCCC
GGCGACGTGCCCAAGTACGACATCATCTTCGTGAACGTGAGGACCCCTTACAAGTACCACC
ACTACCAGCAGTGCGAGGACCACGCCATCAAGCTGAGCATGCTGACCAAGAAGGCCTGCCT
GCACCTGAACCCCGGCGGCACCTGCGTGAGCATCGGCTACGGCTACGCCGACAGGGCCAGC
GAGAGCATCATCGGCGCCATCGCCAGGCTGTTCAAGTTCAGCAGGGTGTGCAAGCCCAAGA
GCAGCCTGGAGGAGACAGAGGTGCTGTTCGTGTTCATCGGCTACGACCGGAAGGCCAGGAC
CCACAACCCCTACAAGCTGAGCAGCACCCTGACCAACATCTACACCGGCAGCAGGCTGCAC
GAGGCCGGCTGCGCCCCTAGCTACCACGTGGTGAGGGGCGACATCGCCACCGCCACCGAGG
GCGTGATCATCAACGCCGCCAACAGCAAGGGCCAGCCCGGCGGCGGGGTGTGCGGCGCCCT
GTATAAGAAGTTCCCCGAGAGCTTCGACCTGCAGCCCATCGAGGTGGGCAAGGCCAGGCTG
GTGAAGGGCGCCGCCAAGCACATCATCCACGCCGTGGGCCCCAACTTCAACAAGGTGAGCG
AGGTGGAGGGCGACAAGCAGCTGGCCGAGGCCTACGAGAGCATCGCCAAGATCGTGAACG
ACAACAACTACAAGAGCGTGGCCATCCCTCTGCTGAGCACCGGCATCTTCAGCGGCAACAA
GGACAGGCTGACCCAGAGCCTGAACCACCTGCTGACCGCCCTGGACACCACCGACGCCGAC
GTGGCCATCTACTGCAGGGACAAGAAGTGGGAGATGACCCTGAAGGAGGCCGTGGCCAGG
CGGGAGGCCGTGGAGGAGATCTGCATCAGCGACGACAGCAGCGTGACGGAGCCCGACGCC
GAGCTGGTGAGGGTGCACCCCAAGAGCAGCCTGGCCGGCAGGAAGGGCTACAGCACCAGC
GACGGCAAGACCTTCAGCTACCTGGAGGGCACCAAGTTCCACCAGGCCGCCAAGGACATCG
CCGAGATCAACGCCATGTGGCCCGTGGCCACCGAGGCCAACGAGCAGGTGTGCATGTATAT
CCTGGGCGAGAGCATGAGCAGCATCAGGAGCAAGTGCCCCGTGGAGGAGAGCGAGGCCAG
CACCCCTCCCAGCACCCTGCCCTGCCTGTGCATCCACGCCATGACCCCTGAGAGGGTGCAGC
GGCTGAAGGCCAGCAGGCCCGAGCAGATCACCGTGTGCAGCAGCTTCCCTCTGCCCAAGTA
CCGGATCACCGGCGTGCAGAAGATCCAGTGCAGCCAGCCCATCCTGTTCAGCCCCAAGGTG
CCCGCCTACATCCACCCCAGGAAGTACCTGGTGGAGACACCCCCCGTGGACGAGACACCCG
AGCCCAGCGCCGAGAACCAGAGCACCGAGGGCACCCCTGAGCAGCCTCCCCTGATCACCGA
GGACGAGACAAGGACCAGGACGCCCGAGCCCATCATCATTGAGGAGGAAGAGGAGGACAG
CATCAGCCTGCTGAGCGACGGCCCCACCCACCAGGTGCTGCAGGTGGAGGCCGACATCCAC
GGCCCTCCCAGCGTGAGCAGCTCCAGCTGGAGCATCCCTCACGCCAGCGACTTCGACGTGG
ACAGCCTGAGCATCCTGGACACCCTGGAGGGCGCCAGCGTGACCAGCGGCGCCACCAGCGC
CGAGACAAACAGCTACTTCGCCAAGAGCATGGAGTTCCTGGCCAGGCCCGTGCCCGCCCCT
AGGACCGTGTTCAGGAACCCTCCCCACCCCGCCCCTAGGACCAGGACCCCTAGCCTGGCCC
CTAGCAGGGCCTGCAGCAGGACCAGCCTGGTGAGCACCCCTCCCGGCGTGAACCGGGTGAT
CACCAGGGAGGAGCTGGAGGCCCTGACCCCTAGCAGGACCCCTAGCAGGAGCGTGAGCAG
GACCAGCCTGGTGAGCAACCCTCCCGGCGTGAACCGGGTGATCACCAGGGAGGAGTTCGAG
GCCTTCGTGGCCCAGCAGCAAAGGCGGTTCGACGCCGGCGCCTACATCTTCAGCAGCGACA
CCGGCCAGGGCCACCTGCAGCAGAAGTCCGTGAGGCAGACCGTGCTGAGCGAGGTGGTCCT
GGAGAGGACGGAGCTGGAGATCAGCTACGCCCCTAGGCTGGACCAGGAGAAGGAGGAGCT
GCTGAGGAAGAAGCTGCAGCTGAACCCCACCCCTGCCAACAGGAGCAGGTACCAGAGCAG
GAAGGTGGAGAACATGAAGGCCATCACCGCCAGGCGGATCCTGCAGGGCCTGGGCCACTAC
CTGAAGGCCGAGGGCAAGGTGGAGTGCTACAGGACCCTGCACCCCGTGCCCCTGTACTCCA
GCTCCGTGAACAGGGCCTTCAGCAGCCCCAAGGTGGCCGTGGAGGCCTGCAACGCCATGCT
GAAGGAGAACTTCCCCACCGTGGCCAGCTACTGCATCATCCCCGAGTACGACGCCTACCTG
GACATGGTGGACGGCGCCAGCTGCTGCCTGGACACCGCCAGCTTCTGCCCCGCCAAGCTGA
GGAGCTTCCCCAAGAAGCACAGCTACCTGGAGCCCACCATCAGGAGCGCCGTGCCCAGCGC
CATCCAGAACACCCTGCAGAACGTGCTGGCCGCCGCTACCAAGAGGAACTGCAACGTGACC
CAGATGAGGGAGCTGCCCGTGCTGGACAGCGCCGCCTTCAACGTGGAGTGCTTCAAGAAGT
ACGCCTGCAACAACGAGTACTGGGAGACATTCAAGGAGAACCCCATCAGGCTGACCGAGGA
GAACGTGGTGAACTACATCACCAAGCTGAAGGGCCCCAAGGCCGCCGCTCTGTTCGCCAAG
ACCCACAACCTGAACATGCTCCAGGACATCCCTATGGACAGGTTCGTGATGGACCTGAAGA
GGGACGTGAAGGTGACCCCTGGCACCAAGCACACCGAGGAGAGGCCCAAGGTGCAGGTGA
TCCAGGCCGCCGACCCTCTGGCCACCGCCTACCTGTGCGGCATCCACAGGGAGCTGGTGAG
GCGGCTGAACGCCGTCCTGCTGCCCAACATCCACACCCTGTTCGACATGAGCGCCGAGGAC
TTCGACGCCATCATCGCCGAGCACTTCCAGCCCGGCGACTGCGTGCTGGAGACAGACATCG
CCAGCTTCGACAAGAGCGAGGACGACGCTATGGCCCTGACCGCCCTGATGATCCTGGAGGA
CCTGGGCGTGGACGCCGAGCTGCTGACCCTGATCGAGGCCGCCTTCGGCGAGATCAGCAGC
ATCCACCTGCCCACCAAGACCAAGTTCAAGTTCGGCGCCATGATGAAGTCCGGCATGTTCCT
GACCCTGTTCGTGAACACCGTGATCAACATCGTGATCGCCAGCAGGGTGCTGCGGGAGAGG
CTGACCGGCAGCCCCTGCGCCGCCTTCATCGGCGACGACAACATCGTGAAGGGCGTGAAGT
CCGACAAGCTGATGGCCGACAGGTGCGCCACCTGGCTGAACATGGAGGTGAAGATCATCGA
CGCCGTGGTGGGCGAGAAGGCCCCTTACTTCTGCGGCGGCTTCATCCTGTGCGACAGCGTGA
CCGGCACCGCCTGCAGGGTGGCCGACCCTCTGAAGAGGCTGTTCAAGCTGGGCAAGCCCCT
GGCCGCCGACGACGAGCACGACGACGATAGGCGGAGGGCCCTGCACGAGGAGAGCACCAG
GTGGAACCGGGTGGGCATCCTGAGCGAGCTGTGCAAGGCCGTGGAGAGCAGGTACGAGAC
AGTGGGCACCAGCATCATCGTGATGGCCATGACCACCCTGGCCAGCAGCGTCAAGTCCTTC
AGCTACCTGAGGGGGGCCCCTATAACTCTCTACGGCTAACCTGAATGGACTACGACATAGTC
TAGTCCGCCAAGGCCCTTAAGTTAAAACAGCTGTGGGTTGTCACCCACCCACAGGGTCCACT
GGGCGCTAGTACACTGGTATCTCGGTACCTTTGTACGCCTGTTTTATACCCCCTCCCTGATTT
GCAACTTAGAAGCAACGCAAACCAGATCAATAGTAGGTGTGACATACCAGTCGCATCTTGA
TCAAGCACTTCTGTATCCCCGGACCGAGTATCAATAGACTGTGCACACGGTTGAAGGAGAA
AACGTCCGTTACCCGGCTAACTACTTCGAGAAGCCTAGTAACGCCATTGAAGTTGCAGAGTG
TTTCGCTCAGCACTCCCCCCGTGTAGATCAGGTCGATGAGTCACCGCATTCCCCACGGGCGA
CCGTGGCGGTGGCTGCGTTGGCGGCCTGCCTATGGGGTAACCCATAGGACGCTCTAATACG
GACATGGCGTGAAGAGTCTATTGAGCTAGTTAGTAGTCCTCCGGCCCCTGAATGCGGCTAAT
CCTAACTGCGGAGCACATACCCTTAATCCAAAGGGCAGTGTGTCGTAACGGGCAACTCTGC
AGCGGAACCGACTACTTTGGGTGTCCGTGTTTCTTTTTATTCTTGTATTGGCTGCTTATGGTG
ACAATTAAAGAATTGTTACCATATAGCTATTGGATTGGCCATCCAGTGTCAAACAGAGCTAT
TGTATATCTCTTTGTTGGATTCACACCTCTCACTCTTGAAACGTTACACACCCTCAATTACAT
TATACTGCTGAACACGAAGCGATGAATCCTAACCAGAAGATCATCACCATCGGCAGCGTGA
GCCTGACCATCAGCACCATCTGCTTCTTCATGCAGATCGCCATCCTGATCACCACCGTGACC
CTGCACTTCAAGCAGTACGAGTTCAACAGCCCTCCCAACAACCAGGTGATGCTGTGCGAGC
CCACCATCATCGAGAGGAACATGACCGAGATCGTGTACCTGACCAACACCACCATCGAGAA
GGAGATCTGCCCCAAGCCCGCCGAGTACAGGAACTGGAGCAAGCCCCAGTGCGGCATCACC
GGCTTCGCCCCTTTCAGCAAGGACAACAGCATCAGGCTGAGCGCCGGCGGCGACATCTGGG
TGACCAGGGAGCCCTACGTGAGCTGCGACCTGGACAAGTGCTACCAGTTCGCCCTGGGCCA
GGGCACCACCCTGAACAACGTGCACAGCAACAACACCGTGAGGGACAGGACCCCTTACCGG
ACCCTGCTGATGAACGAGCTGGGCGTGCCCTTCCACCTGGGCACCAAGCAGGTGTGCATCG
CCTGGAGCAGCTCCAGCTGCCACGACGGCAAGGCCTGGCTGCACGTGTGCATCACCGGCGA
CGACAAGAACGCCACCGCCAGCTTCATCTACAACGGCAGGCTGGTGGACAGCGTGGTGAGC
TGGAGCAACGACATCCTGAGGACCCAGGAGAGCGAGTGCGTGTGCATCAACGGCACCTGCA
CCGTGGTGATGACCGACGGCAACGCCACCGGCAAGGCCGACACCAAGATCCTGTTCATCGA
GGAGGGCAAGATCGTGCACACCAGCAAGCTGAGCGGCAGCGCCCAGCACGTGGAGGAGTG
CAGCTGCTACCCCAGGTACCCCGGCGTGAGGTGCGTGTGCAGGGACAACTGGAAGGGCAGC
AACCGGCCCATCATCGACATCAACATCAAGGACCACAGCATCGTCAGCAGGTACGTGTGCA
GCGGCCTGGTGGGCGACACCCCTAGAAAGAGCGACAGCAGCTCCAGCAGCCACTGCCTGAA
CCCCAACAACGAGAAGGGCGACCACGGCGTGAAGGGCTGGGCCTTCGACGACGGCAACGA
CGTGTGGATGGGCAGGACCATCAACGAGACAAGCAGGCTGGGCTACGAGACATTCAAGGTG
GTGGAGGGCTGGAGCAACCCCAAGAGCAAGCTGCAGATCAACAGGCAGGTGATCGTGGAC
AGGGGCGACAGGAGCGGCTACAGCGGCATCTTCAGCGTGGAGGGCAAGAGCTGCATCAAC
CGGTGCTTCTACGTGGAGCTGATCAGGGGCAGGAAGGAGGAGACAGAGGTGCTGTGGACCA
GCAACAGCATCGTGGTGTTCTGCGGCACCAGCGGCACCTACGGCACCGGCAGCTGGCCCGA
CGGCGCCAACCTGAGCCTGATGCACATCTGATAGTAAACTCGAAGCAGTGTTAAATCATTCA
GCTACCTGAGAGGGGCCCCTATAACTCTCTACGGCTAACCTGAATGGACTACGACATAGTCT
AGTCCGCCAAGGCCGAATTCTTAAAACAGCTGTGGGTTGTCACCCACCCACAGGGTCCACTG
GGCGCTAGTACACTGGTATCTCGGTACCTTTGTACGCCTGTTTTATACCCCCTCCCTGATTTG
CAACTTAGAAGCAACGCAAACCAGATCAATAGTAGGTGTGACATACCAGTCGCATCTTGAT
CAAGCACTTCTGTATCCCCGGACCGAGTATCAATAGACTGTGCACACGGTTGAAGGAGAAA
ACGTCCGTTACCCGGCTAACTACTTCGAGAAGCCTAGTAACGCCATTGAAGTTGCAGAGTGT
TTCGCTCAGCACTCCCCCCGTGTAGATCAGGTCGATGAGTCACCGCATTCCCCACGGGCGAC
CGTGGCGGTGGCTGCGTTGGCGGCCTGCCTATGGGGTAACCCATAGGACGCTCTAATACGG
ACATGGCGTGAAGAGTCTATTGAGCTAGTTAGTAGTCCTCCGGCCCCTGAATGCGGCTAATC
CTAACTGCGGAGCACATACCCTTAATCCAAAGGGCAGTGTGTCGTAACGGGCAACTCTGCA
GCGGAACCGACTACTTTGGGTGTCCGTGTTTCTTTTTATTCTTGTATTGGCTGCTTATGGTGA
CAATTAAAGAATTGTTACCATATAGCTATTGGATTGGCCATCCAGTGTCAAACAGAGCTATT
GTATATCTCTTTGTTGGATTCACACCTCTCACTCTTGAAACGTTACACACCCTCAATTACATT
ATACTGCTGAACACGAAGCGATGAAGACCATCATCGCCCTGAGCTACATCCTGTGCCTGGTG
TTCGCCCAGAAGATCCCCGGCAATGACAACAGCACCGCCACCCTGTGCCTGGGCCACCACG
CCGTGCCCAACGGCACCATCGTGAAGACCATCACCAACGACAGGATCGAGGTGACCAACGC
CACCGAGCTGGTGCAGAACAGCAGCATCGGCGAGATCTGCGACAGCCCTCACCAGATCCTG
GACGGCGGCAACTGCACCCTGATCGACGCCCTGCTGGGCGACCCTCAGTGCGACGGCTTCC
AGAACAAGGAGTGGGACCTGTTCGTGGAGAGGAGCAGGGCCAACAGCAACTGCTACCCCTA
CGACGTGCCCGACTACGCCAGCCTGAGGAGCCTGGTGGCCAGCAGCGGCACCCTGGAGTTC
AAGAACGAGAGCTTCAACTGGACCGGCGTGAAGCAGAACGGCACCAGCAGCGCCTGCATC
AGGGGCAGCAGCTCCAGCTTCTTCAGCCGGCTGAATTGGCTCACCCACCTGAACTACACCTA
CCCCGCCCTGAACGTGACCATGCCCAACAACGAGCAGTTCGACAAGCTGTATATCTGGGGC
GTGCACCACCCCAGCACCGACAAGGACCAGATCAGCCTGTTCGCCCAGCCCAGCGGCAGGA
TCACCGTGAGCACCAAGAGGAGCCAGCAGGCCGTGATCCCCAACATCGGCAGCAGGCCCAG
GATCAGGGACATCCCCAGCAGGATCAGCATCTACTGGACCATCGTGAAGCCCGGCGACATC
CTGCTGATCAACAGCACCGGCAACCTGATCGCCCCTAGGGGCTACTTCAAGATCAGGAGCG
GCAAGAGCAGCATCATGAGGAGCGACGCCCCTATCGGCAAGTGCAAGAGCGAGTGCATCAC
CCCTAACGGCAGCATCCCCAACGACAAGCCCTTCCAGAACGTGAACAGGATCACCTACGGC
GCCTGCCCCAGGTACGTGAAGCAGAGCACCCTGAAGCTGGCCACCGGCATGAGGAACGTGC
CCGAGAAGCAGACCAGGGGCATCTTCGGCGCCATCGCCGGCTTCATCGAGAACGGCTGGGA
GGGCATGGTGGACGGCTGGTACGGCTTCAGGCACCAGAACAGCGAGGGCAGGGGCCAGGC
CGCCGACCTGAAGTCCACCCAGGCCGCCATCGACCAGATCAACGGCAAGCTGAACAGGCTG
ATCGGCAAGACCAACGAGAAGTTCCACCAGATCGAGAAGGAGTTCAGCGAGGTGGAGGGC
AGGGTGCAGGACCTGGAGAAGTACGTGGAGGACACCAAGATCGACCTGTGGAGCTACAAC
GCCGAGCTGCTGGTGGCCCTGGAGAACCAGCACACCATCGACCTGACCGACAGCGAGATGA
ACAAGCTGTTCGAGAAGACCAAGAAGCAGCTGAGGGAGAACGCCGAGGACATGGGCAACG
GCTGCTTCAAGATCTACCACAAGTGCGACAACGCCTGCATCGGCAGCATCAGGAACGAGAC
ATATGACCACAACGTGTACCGGGACGAGGCCCTGAACAACCGGTTCCAGATCAAGGGCGTG
GAGCTGAAGTCCGGCTACAAGGACTGGATCCTGTGGATCAGCTTCGCCATGAGCTGCTTCCT
GCTGTGCATCGCCCTGCTGGGCTTCATCATGTGGGCCTGCCAGAAGGGCAACATCAGGTGCA
ACATCTGCATCTGATAGTAAACTCGAGTATGTTACGTGCAAAGGTGATTGTCACCCCCCGAA
AGACCATATTGTGACACACCCTCAGTATCACGCCCAAACATTTACAGCCGCGGTGTCAAAA
ACCGCGTGGACGTGGTTAACATCCCTGCTGGGAGGATCAGCCGTAATTATTATAATTGGCTT
GGTGCTGGCTACTATTGTGGCCATGTACGTGCTGACCAACCAGAAACATAATTGAATACAGC
AGCAATTGGCAAGCTGCTTACATAGAACTCGCGGCGATTGGCATGCCGCCTTAAAATTTTTA
TTTTATTTTTTCTTTTCTTTTCCGAATCGGATTTTGTTTTTAATATTTC
SEQ ID NO: 159 (sgP CVB3 NA sgP CVB3 HA)
ATGGGCGGCGCATGAGAGAAGCCCAGACCAATTACCTACCCAAAATGGAGAAAGTTCACGT
TGACATCGAGGAAGACAGCCCATTCCTCAGAGCTTTGCAGCGGAGCTTCCCGCAGTTTGAG
GTAGAAGCCAAGCAGGTCACTGATAATGACCATGCTAATGCCAGAGCGTTTTCGCATCTGG
CTTCAAAACTGATCGAAACGGAGGTGGACCCATCCGACACGATCCTTGACATTGGAAGTGC
GCCCGCCCGCAGAATGTATTCTAAGCACAAGTATCATTGTATCTGTCCGATGAGATGTGCGG
AAGATCCGGACAGATTGTATAAGTATGCAACTAAGCTGAAGAAAAACTGTAAGGAAATAAC
TGATAAGGAATTGGACAAGAAAATGAAGGAGCTGGCCGCCGTCATGAGCGACCCTGACCTG
GAAACTGAGACTATGTGCCTCCACGACGACGAGTCGTGTCGCTACGAAGGGCAAGTCGCTG
TTTACCAGGATGTATACGCCGTCGACGGCCCCACCAGCCTGTACCACCAGGCCAACAAGGG
CGTGAGGGTGGCCTACTGGATCGGCTTCGACACCACACCCTTCATGTTCAAGAACCTGGCCG
GCGCCTACCCCAGCTACAGCACCAACTGGGCCGACGAGACAGTGCTGACCGCCAGGAACAT
CGGCCTGTGCAGCAGCGACGTGATGGAGAGGAGCCGGAGGGGCATGAGCATCCTGAGGAA
GAAGTACCTGAAGCCCAGCAACAACGTGCTGTTCAGCGTGGGCAGCACCATCTACCACGAG
AAGAGGGACCTGCTGAGGAGCTGGCACCTGCCCAGCGTGTTCCACCTGAGGGGCAAGCAGA
ACTACACCTGCAGGTGCGAGACAATCGTGAGCTGCGACGGCTACGTGGTGAAGAGGATCGC
CATCAGCCCCGGCCTGTACGGCAAGCCCAGCGGCTACGCCGCCACCATGCACAGGGAGGGC
TTCCTGTGCTGCAAGGTGACCGACACCCTGAACGGCGAGAGGGTGAGCTTCCCCGTGTGCA
CCTACGTGCCCGCCACCCTGTGCGACCAGATGACCGGCATCCTGGCCACCGACGTGAGCGC
CGACGACGCCCAGAAGCTGCTGGTGGGCCTGAACCAGAGGATCGTGGTGAACGGCAGGACC
CAGAGGAACACCAACACCATGAAGAACTACCTGCTGCCCGTGGTGGCCCAGGCCTTCGCCA
GGTGGGCCAAGGAGTACAAGGAGGACCAGGAGGACGAGAGGCCCCTGGGCCTGAGGGACC
GACAGCTGGTGATGGGCTGCTGCTGGGCCTTCAGGCGGCACAAGATCACCAGCATCTACAA
GAGGCCCGACACCCAGACCATCATCAAGGTGAACAGCGACTTCCACAGCTTCGTGCTGCCC
AGGATCGGCAGCAACACCCTGGAGATCGGCCTGAGGACCCGGATCAGGAAGATGCTGGAG
GAGCACAAGGAGCCCAGCCCTCTGATCACCGCCGAGGACGTGCAGGAGGCCAAGTGCGCC
GCCGACGAGGCCAAGGAGGTGAGGGAGGCCGAGGAGCTGAGGGCCGCCCTGCCTCCCCTG
GCCGCCGACGTGGAGGAGCCCACCCTGGAGGCCGACGTGGACCTGATGCTGCAGGAGGCCG
GCGCCGGCAGCGTGGAGACACCCAGGGGCCTGATCAAGGTGACCAGCTACGACGGCGAGG
ACAAGATCGGCAGCTACGCCGTGCTCAGCCCTCAGGCCGTGCTGAAGTCCGAGAAGCTGAG
CTGCATCCACCCTCTGGCCGAGCAGGTGATCGTGATCACCCACAGCGGCAGGAAGGGCAGG
TACGCCGTGGAGCCCTACCACGGCAAGGTGGTGGTCCCCGAGGGCCACGCCATCCCCGTGC
AGGACTTCCAGGCCCTGAGCGAGAGCGCCACCATCGTGTATAACGAGAGGGAGTTCGTGAA
CAGGTACCTGCACCACATCGCCACCCACGGCGGCGCCCTGAACACCGACGAGGAGTACTAC
AAGACCGTGAAGCCCAGCGAGCACGACGGCGAGTACCTGTACGACATCGACAGGAAGCAG
TGCGTGAAGAAGGAGCTGGTGACCGGCCTGGGCCTGACCGGCGAGCTGGTGGACCCTCCCT
TCCACGAGTTCGCCTACGAGAGCCTGAGGACCAGGCCCGCCGCTCCCTACCAGGTGCCCAC
CATCGGCGTGTACGGCGTGCCCGGCAGCGGCAAGAGCGGCATCATCAAGAGCGCCGTGACC
AAGAAGGACCTGGTGGTGAGCGCCAAGAAGGAGAACTGCGCCGAGATCATCAGGGACGTG
AAGAAGATGAAGGGCCTGGACGTGAACGCCAGGACCGTGGACAGCGTGCTCCTGAACGGCT
GCAAGCACCCCGTGGAGACACTGTATATCGACGAGGCCTTCGCCTGCCACGCCGGCACCCT
GAGGGCCCTGATCGCCATCATCAGGCCCAAGAAGGCCGTGCTGTGCGGCGACCCCAAGCAG
TGCGGCTTCTTCAACATGATGTGCCTGAAGGTGCACTTCAACCACGAGATCTGCACCCAGGT
GTTCCACAAGAGCATCAGCAGGCGGTGCACCAAGAGCGTGACCAGCGTGGTGAGCACCCTG
TTCTACGACAAGAAGATGAGGACCACCAACCCCAAGGAGACAAAGATCGTGATCGACACCA
CCGGCAGCACCAAGCCCAAGCAGGACGACCTGATCCTGACCTGCTTCAGGGGCTGGGTGAA
GCAGCTGCAGATCGACTACAAGGGCAACGAGATCATGACCGCCGCCGCTAGCCAGGGCCTG
ACCAGGAAGGGCGTGTACGCCGTGAGGTACAAGGTGAACGAGAATCCCCTGTACGCCCCTA
CCAGCGAGCACGTGAACGTCCTGCTGACCAGGACCGAGGACAGGATCGTGTGGAAGACCCT
GGCCGGCGACCCCTGGATCAAGACCCTGACCGCCAAGTACCCCGGCAACTTCACCGCCACC
ATCGAGGAGTGGCAGGCCGAGCACGACGCCATCATGAGGCACATCCTGGAGAGGCCCGACC
CCACCGACGTGTTCCAGAACAAGGCCAACGTGTGCTGGGCCAAGGCCCTGGTGCCCGTGCT
GAAGACCGCCGGCATCGACATGACCACCGAGCAGTGGAACACCGTGGACTACTTCGAGACA
GACAAGGCCCACAGCGCCGAGATCGTGCTGAACCAGCTGTGCGTGAGGTTCTTCGGCCTGG
ACCTGGACAGCGGCCTGTTCAGCGCCCCTACCGTGCCCCTGAGCATCAGGAACAACCACTG
GGACAACAGCCCCAGCCCCAACATGTACGGCCTGAACAAGGAGGTGGTGAGGCAGCTGAG
CAGGCGGTACCCTCAGCTGCCCAGGGCCGTGGCCACCGGCAGGGTGTACGACATGAACACC
GGCACCCTGAGGAACTACGACCCCAGGATCAACCTGGTGCCCGTGAACAGGCGGCTGCCAC
ACGCCCTGGTGCTGCACCACAACGAGCACCCTCAGAGCGACTTCAGCAGCTTCGTGAGCAA
GCTGAAGGGCAGGACCGTGCTGGTGGTGGGCGAGAAGCTGAGCGTGCCCGGCAAGATGGT
GGACTGGCTGAGCGACAGGCCCGAGGCCACCTTCCGGGCCAGGCTGGACCTGGGCATCCCC
GGCGACGTGCCCAAGTACGACATCATCTTCGTGAACGTGAGGACCCCTTACAAGTACCACC
ACTACCAGCAGTGCGAGGACCACGCCATCAAGCTGAGCATGCTGACCAAGAAGGCCTGCCT
GCACCTGAACCCCGGCGGCACCTGCGTGAGCATCGGCTACGGCTACGCCGACAGGGCCAGC
GAGAGCATCATCGGCGCCATCGCCAGGCTGTTCAAGTTCAGCAGGGTGTGCAAGCCCAAGA
GCAGCCTGGAGGAGACAGAGGTGCTGTTCGTGTTCATCGGCTACGACCGGAAGGCCAGGAC
CCACAACCCCTACAAGCTGAGCAGCACCCTGACCAACATCTACACCGGCAGCAGGCTGCAC
GAGGCCGGCTGCGCCCCTAGCTACCACGTGGTGAGGGGCGACATCGCCACCGCCACCGAGG
GCGTGATCATCAACGCCGCCAACAGCAAGGGCCAGCCCGGCGGCGGGGTGTGCGGCGCCCT
GTATAAGAAGTTCCCCGAGAGCTTCGACCTGCAGCCCATCGAGGTGGGCAAGGCCAGGCTG
GTGAAGGGCGCCGCCAAGCACATCATCCACGCCGTGGGCCCCAACTTCAACAAGGTGAGCG
AGGTGGAGGGCGACAAGCAGCTGGCCGAGGCCTACGAGAGCATCGCCAAGATCGTGAACG
ACAACAACTACAAGAGCGTGGCCATCCCTCTGCTGAGCACCGGCATCTTCAGCGGCAACAA
GGACAGGCTGACCCAGAGCCTGAACCACCTGCTGACCGCCCTGGACACCACCGACGCCGAC
GTGGCCATCTACTGCAGGGACAAGAAGTGGGAGATGACCCTGAAGGAGGCCGTGGCCAGG
CGGGAGGCCGTGGAGGAGATCTGCATCAGCGACGACAGCAGCGTGACGGAGCCCGACGCC
GAGCTGGTGAGGGTGCACCCCAAGAGCAGCCTGGCCGGCAGGAAGGGCTACAGCACCAGC
GACGGCAAGACCTTCAGCTACCTGGAGGGCACCAAGTTCCACCAGGCCGCCAAGGACATCG
CCGAGATCAACGCCATGTGGCCCGTGGCCACCGAGGCCAACGAGCAGGTGTGCATGTATAT
CCTGGGCGAGAGCATGAGCAGCATCAGGAGCAAGTGCCCCGTGGAGGAGAGCGAGGCCAG
CACCCCTCCCAGCACCCTGCCCTGCCTGTGCATCCACGCCATGACCCCTGAGAGGGTGCAGC
GGCTGAAGGCCAGCAGGCCCGAGCAGATCACCGTGTGCAGCAGCTTCCCTCTGCCCAAGTA
CCGGATCACCGGCGTGCAGAAGATCCAGTGCAGCCAGCCCATCCTGTTCAGCCCCAAGGTG
CCCGCCTACATCCACCCCAGGAAGTACCTGGTGGAGACACCCCCCGTGGACGAGACACCCG
AGCCCAGCGCCGAGAACCAGAGCACCGAGGGCACCCCTGAGCAGCCTCCCCTGATCACCGA
GGACGAGACAAGGACCAGGACGCCCGAGCCCATCATCATTGAGGAGGAAGAGGAGGACAG
CATCAGCCTGCTGAGCGACGGCCCCACCCACCAGGTGCTGCAGGTGGAGGCCGACATCCAC
GGCCCTCCCAGCGTGAGCAGCTCCAGCTGGAGCATCCCTCACGCCAGCGACTTCGACGTGG
ACAGCCTGAGCATCCTGGACACCCTGGAGGGCGCCAGCGTGACCAGCGGCGCCACCAGCGC
CGAGACAAACAGCTACTTCGCCAAGAGCATGGAGTTCCTGGCCAGGCCCGTGCCCGCCCCT
AGGACCGTGTTCAGGAACCCTCCCCACCCCGCCCCTAGGACCAGGACCCCTAGCCTGGCCC
CTAGCAGGGCCTGCAGCAGGACCAGCCTGGTGAGCACCCCTCCCGGCGTGAACCGGGTGAT
CACCAGGGAGGAGCTGGAGGCCCTGACCCCTAGCAGGACCCCTAGCAGGAGCGTGAGCAG
GACCAGCCTGGTGAGCAACCCTCCCGGCGTGAACCGGGTGATCACCAGGGAGGAGTTCGAG
GCCTTCGTGGCCCAGCAGCAAAGGCGGTTCGACGCCGGCGCCTACATCTTCAGCAGCGACA
CCGGCCAGGGCCACCTGCAGCAGAAGTCCGTGAGGCAGACCGTGCTGAGCGAGGTGGTCCT
GGAGAGGACGGAGCTGGAGATCAGCTACGCCCCTAGGCTGGACCAGGAGAAGGAGGAGCT
GCTGAGGAAGAAGCTGCAGCTGAACCCCACCCCTGCCAACAGGAGCAGGTACCAGAGCAG
GAAGGTGGAGAACATGAAGGCCATCACCGCCAGGCGGATCCTGCAGGGCCTGGGCCACTAC
CTGAAGGCCGAGGGCAAGGTGGAGTGCTACAGGACCCTGCACCCCGTGCCCCTGTACTCCA
GCTCCGTGAACAGGGCCTTCAGCAGCCCCAAGGTGGCCGTGGAGGCCTGCAACGCCATGCT
GAAGGAGAACTTCCCCACCGTGGCCAGCTACTGCATCATCCCCGAGTACGACGCCTACCTG
GACATGGTGGACGGCGCCAGCTGCTGCCTGGACACCGCCAGCTTCTGCCCCGCCAAGCTGA
GGAGCTTCCCCAAGAAGCACAGCTACCTGGAGCCCACCATCAGGAGCGCCGTGCCCAGCGC
CATCCAGAACACCCTGCAGAACGTGCTGGCCGCCGCTACCAAGAGGAACTGCAACGTGACC
CAGATGAGGGAGCTGCCCGTGCTGGACAGCGCCGCCTTCAACGTGGAGTGCTTCAAGAAGT
ACGCCTGCAACAACGAGTACTGGGAGACATTCAAGGAGAACCCCATCAGGCTGACCGAGGA
GAACGTGGTGAACTACATCACCAAGCTGAAGGGCCCCAAGGCCGCCGCTCTGTTCGCCAAG
ACCCACAACCTGAACATGCTCCAGGACATCCCTATGGACAGGTTCGTGATGGACCTGAAGA
GGGACGTGAAGGTGACCCCTGGCACCAAGCACACCGAGGAGAGGCCCAAGGTGCAGGTGA
TCCAGGCCGCCGACCCTCTGGCCACCGCCTACCTGTGCGGCATCCACAGGGAGCTGGTGAG
GCGGCTGAACGCCGTCCTGCTGCCCAACATCCACACCCTGTTCGACATGAGCGCCGAGGAC
TTCGACGCCATCATCGCCGAGCACTTCCAGCCCGGCGACTGCGTGCTGGAGACAGACATCG
CCAGCTTCGACAAGAGCGAGGACGACGCTATGGCCCTGACCGCCCTGATGATCCTGGAGGA
CCTGGGCGTGGACGCCGAGCTGCTGACCCTGATCGAGGCCGCCTTCGGCGAGATCAGCAGC
ATCCACCTGCCCACCAAGACCAAGTTCAAGTTCGGCGCCATGATGAAGTCCGGCATGTTCCT
GACCCTGTTCGTGAACACCGTGATCAACATCGTGATCGCCAGCAGGGTGCTGCGGGAGAGG
CTGACCGGCAGCCCCTGCGCCGCCTTCATCGGCGACGACAACATCGTGAAGGGCGTGAAGT
CCGACAAGCTGATGGCCGACAGGTGCGCCACCTGGCTGAACATGGAGGTGAAGATCATCGA
CGCCGTGGTGGGCGAGAAGGCCCCTTACTTCTGCGGCGGCTTCATCCTGTGCGACAGCGTGA
CCGGCACCGCCTGCAGGGTGGCCGACCCTCTGAAGAGGCTGTTCAAGCTGGGCAAGCCCCT
GGCCGCCGACGACGAGCACGACGACGATAGGCGGAGGGCCCTGCACGAGGAGAGCACCAG
GTGGAACCGGGTGGGCATCCTGAGCGAGCTGTGCAAGGCCGTGGAGAGCAGGTACGAGAC
AGTGGGCACCAGCATCATCGTGATGGCCATGACCACCCTGGCCAGCAGCGTCAAGTCCTTC
AGCTACCTGAGGGGGGCCCCTATAACTCTCTACGGCTAACCTGAATGGACTACGACATAGTC
TAGTCCGCCAAGGCCCTTAAGTTAAAACAGCCTGTGGGTTGATCCCACCCACAGGCCCATTG
GGCGCTAGCACTCTGGTATCACGGTACCTTTGTGCGCCTGTTTTATACCCCCTCCCCCAACTG
TAACTTAGAAGTAACACACACCGATCAACAGTCAGCGTGGCACACCAGCCACGTTTTGATC
AAGCACTTCTGTTACCCCGGACTGAGTATCAATAGACTGCTCACGCGGTTGAAGGAGAAAG
CGTTCGTTATCCGGCCAACTACTTCGAAAAACCTAGTAACACCGTGGAAGTTGCAGAGTGTT
TCGCTCAGCACTACCCCAGTGTAGATCAGGTCGATGAGTCACCGCATTCCCCACGGGCGACC
GTGGCGGTGGCTGCGTTGGCGGCCTGCCCATGGGGAAACCCATGGGACGCTCTAATACAGA
CATGGTGCGAAGAGTCTATTGAGCTAGTTGGTAGTCCTCCGGCCCCTGAATGCGGCTAATCC
TAACTGCGGAGCACACACCCTCAAGCCAGAGGGCAGTGTGTCGTAACGGGCAACTCTGCAG
CGGAACCGACTACTTTGGGTGTCCGTGTTTCATTTTATTCCTATACTGGCTGCTTATGGTGAC
AATTGAGAGATCGTTACCATATAGCTATTGGATTGGCCATCCGGTGACTAATAGAGCTATTA
TATATCCCTTTGTTGGGTTTATACCACTTAGCTTGAAAGAGGTTAAAACATTACAATTCATTG
TTAAGTTGAATACAGCAAAATGAATCCTAACCAGAAGATCATCACCATCGGCAGCGTGAGC
CTGACCATCAGCACCATCTGCTTCTTCATGCAGATCGCCATCCTGATCACCACCGTGACCCT
GCACTTCAAGCAGTACGAGTTCAACAGCCCTCCCAACAACCAGGTGATGCTGTGCGAGCCC
ACCATCATCGAGAGGAACATGACCGAGATCGTGTACCTGACCAACACCACCATCGAGAAGG
AGATCTGCCCCAAGCCCGCCGAGTACAGGAACTGGAGCAAGCCCCAGTGCGGCATCACCGG
CTTCGCCCCTTTCAGCAAGGACAACAGCATCAGGCTGAGCGCCGGCGGCGACATCTGGGTG
ACCAGGGAGCCCTACGTGAGCTGCGACCTGGACAAGTGCTACCAGTTCGCCCTGGGCCAGG
GCACCACCCTGAACAACGTGCACAGCAACAACACCGTGAGGGACAGGACCCCTTACCGGAC
CCTGCTGATGAACGAGCTGGGCGTGCCCTTCCACCTGGGCACCAAGCAGGTGTGCATCGCCT
GGAGCAGCTCCAGCTGCCACGACGGCAAGGCCTGGCTGCACGTGTGCATCACCGGCGACGA
CAAGAACGCCACCGCCAGCTTCATCTACAACGGCAGGCTGGTGGACAGCGTGGTGAGCTGG
AGCAACGACATCCTGAGGACCCAGGAGAGCGAGTGCGTGTGCATCAACGGCACCTGCACCG
TGGTGATGACCGACGGCAACGCCACCGGCAAGGCCGACACCAAGATCCTGTTCATCGAGGA
GGGCAAGATCGTGCACACCAGCAAGCTGAGCGGCAGCGCCCAGCACGTGGAGGAGTGCAG
CTGCTACCCCAGGTACCCCGGCGTGAGGTGCGTGTGCAGGGACAACTGGAAGGGCAGCAAC
CGGCCCATCATCGACATCAACATCAAGGACCACAGCATCGTCAGCAGGTACGTGTGCAGCG
GCCTGGTGGGCGACACCCCTAGAAAGAGCGACAGCAGCTCCAGCAGCCACTGCCTGAACCC
CAACAACGAGAAGGGCGACCACGGCGTGAAGGGCTGGGCCTTCGACGACGGCAACGACGT
GTGGATGGGCAGGACCATCAACGAGACAAGCAGGCTGGGCTACGAGACATTCAAGGTGGT
GGAGGGCTGGAGCAACCCCAAGAGCAAGCTGCAGATCAACAGGCAGGTGATCGTGGACAG
GGGCGACAGGAGCGGCTACAGCGGCATCTTCAGCGTGGAGGGCAAGAGCTGCATCAACCG
GTGCTTCTACGTGGAGCTGATCAGGGGCAGGAAGGAGGAGACAGAGGTGCTGTGGACCAGC
AACAGCATCGTGGTGTTCTGCGGCACCAGCGGCACCTACGGCACCGGCAGCTGGCCCGACG
GCGCCAACCTGAGCCTGATGCACATCTGATAGTAAACTCGAAGCAGTGTTAAATCATTCAGC
TACCTGAGAGGGGCCCCTATAACTCTCTACGGCTAACCTGAATGGACTACGACATAGTCTAG
TCCGCCAAGGCCGAATTCTTAAAACAGCCTGTGGGTTGATCCCACCCACAGGCCCATTGGGC
GCTAGCACTCTGGTATCACGGTACCTTTGTGCGCCTGTTTTATACCCCCTCCCCCAACTGTAA
CTTAGAAGTAACACACACCGATCAACAGTCAGCGTGGCACACCAGCCACGTTTTGATCAAG
CACTTCTGTTACCCCGGACTGAGTATCAATAGACTGCTCACGCGGTTGAAGGAGAAAGCGTT
CGTTATCCGGCCAACTACTTCGAAAAACCTAGTAACACCGTGGAAGTTGCAGAGTGTTTCGC
TCAGCACTACCCCAGTGTAGATCAGGTCGATGAGTCACCGCATTCCCCACGGGCGACCGTG
GCGGTGGCTGCGTTGGCGGCCTGCCCATGGGGAAACCCATGGGACGCTCTAATACAGACAT
GGTGCGAAGAGTCTATTGAGCTAGTTGGTAGTCCTCCGGCCCCTGAATGCGGCTAATCCTAA
CTGCGGAGCACACACCCTCAAGCCAGAGGGCAGTGTGTCGTAACGGGCAACTCTGCAGCGG
AACCGACTACTTTGGGTGTCCGTGTTTCATTTTATTCCTATACTGGCTGCTTATGGTGACAAT
TGAGAGATCGTTACCATATAGCTATTGGATTGGCCATCCGGTGACTAATAGAGCTATTATAT
ATCCCTTTGTTGGGTTTATACCACTTAGCTTGAAAGAGGTTAAAACATTACAATTCATTGTTA
AGTTGAATACAGCAAAATGAAGACCATCATCGCCCTGAGCTACATCCTGTGCCTGGTGTTCG
CCCAGAAGATCCCCGGCAATGACAACAGCACCGCCACCCTGTGCCTGGGCCACCACGCCGT
GCCCAACGGCACCATCGTGAAGACCATCACCAACGACAGGATCGAGGTGACCAACGCCACC
GAGCTGGTGCAGAACAGCAGCATCGGCGAGATCTGCGACAGCCCTCACCAGATCCTGGACG
GCGGCAACTGCACCCTGATCGACGCCCTGCTGGGCGACCCTCAGTGCGACGGCTTCCAGAA
CAAGGAGTGGGACCTGTTCGTGGAGAGGAGCAGGGCCAACAGCAACTGCTACCCCTACGAC
GTGCCCGACTACGCCAGCCTGAGGAGCCTGGTGGCCAGCAGCGGCACCCTGGAGTTCAAGA
ACGAGAGCTTCAACTGGACCGGCGTGAAGCAGAACGGCACCAGCAGCGCCTGCATCAGGG
GCAGCAGCTCCAGCTTCTTCAGCCGGCTGAATTGGCTCACCCACCTGAACTACACCTACCCC
GCCCTGAACGTGACCATGCCCAACAACGAGCAGTTCGACAAGCTGTATATCTGGGGCGTGC
ACCACCCCAGCACCGACAAGGACCAGATCAGCCTGTTCGCCCAGCCCAGCGGCAGGATCAC
CGTGAGCACCAAGAGGAGCCAGCAGGCCGTGATCCCCAACATCGGCAGCAGGCCCAGGAT
CAGGGACATCCCCAGCAGGATCAGCATCTACTGGACCATCGTGAAGCCCGGCGACATCCTG
CTGATCAACAGCACCGGCAACCTGATCGCCCCTAGGGGCTACTTCAAGATCAGGAGCGGCA
AGAGCAGCATCATGAGGAGCGACGCCCCTATCGGCAAGTGCAAGAGCGAGTGCATCACCCC
TAACGGCAGCATCCCCAACGACAAGCCCTTCCAGAACGTGAACAGGATCACCTACGGCGCC
TGCCCCAGGTACGTGAAGCAGAGCACCCTGAAGCTGGCCACCGGCATGAGGAACGTGCCCG
AGAAGCAGACCAGGGGCATCTTCGGCGCCATCGCCGGCTTCATCGAGAACGGCTGGGAGGG
CATGGTGGACGGCTGGTACGGCTTCAGGCACCAGAACAGCGAGGGCAGGGGCCAGGCCGC
CGACCTGAAGTCCACCCAGGCCGCCATCGACCAGATCAACGGCAAGCTGAACAGGCTGATC
GGCAAGACCAACGAGAAGTTCCACCAGATCGAGAAGGAGTTCAGCGAGGTGGAGGGCAGG
GTGCAGGACCTGGAGAAGTACGTGGAGGACACCAAGATCGACCTGTGGAGCTACAACGCCG
AGCTGCTGGTGGCCCTGGAGAACCAGCACACCATCGACCTGACCGACAGCGAGATGAACAA
GCTGTTCGAGAAGACCAAGAAGCAGCTGAGGGAGAACGCCGAGGACATGGGCAACGGCTG
CTTCAAGATCTACCACAAGTGCGACAACGCCTGCATCGGCAGCATCAGGAACGAGACATAT
GACCACAACGTGTACCGGGACGAGGCCCTGAACAACCGGTTCCAGATCAAGGGCGTGGAGC
TGAAGTCCGGCTACAAGGACTGGATCCTGTGGATCAGCTTCGCCATGAGCTGCTTCCTGCTG
TGCATCGCCCTGCTGGGCTTCATCATGTGGGCCTGCCAGAAGGGCAACATCAGGTGCAACAT
CTGCATCTGATAGTAAACTCGAGTATGTTACGTGCAAAGGTGATTGTCACCCCCCGAAAGAC
CATATTGTGACACACCCTCAGTATCACGCCCAAACATTTACAGCCGCGGTGTCAAAAACCGC
GTGGACGTGGTTAACATCCCTGCTGGGAGGATCAGCCGTAATTATTATAATTGGCTTGGTGC
TGGCTACTATTGTGGCCATGTACGTGCTGACCAACCAGAAACATAATTGAATACAGCAGCA
ATTGGCAAGCTGCTTACATAGAACTCGCGGCGATTGGCATGCCGCCTTAAAATTTTTATTTT
ATTTTTTCTTTTCTTTTCCGAATCGGATTTTGTTTTTAATATTTC
SEQ ID NO: 160 (sgP CVB3 NA sgP EV71 HA)
ATGGGCGGCGCATGAGAGAAGCCCAGACCAATTACCTACCCAAAATGGAGAAAGTTCACGT
TGACATCGAGGAAGACAGCCCATTCCTCAGAGCTTTGCAGCGGAGCTTCCCGCAGTTTGAG
GTAGAAGCCAAGCAGGTCACTGATAATGACCATGCTAATGCCAGAGCGTTTTCGCATCTGG
CTTCAAAACTGATCGAAACGGAGGTGGACCCATCCGACACGATCCTTGACATTGGAAGTGC
GCCCGCCCGCAGAATGTATTCTAAGCACAAGTATCATTGTATCTGTCCGATGAGATGTGCGG
AAGATCCGGACAGATTGTATAAGTATGCAACTAAGCTGAAGAAAAACTGTAAGGAAATAAC
TGATAAGGAATTGGACAAGAAAATGAAGGAGCTGGCCGCCGTCATGAGCGACCCTGACCTG
GAAACTGAGACTATGTGCCTCCACGACGACGAGTCGTGTCGCTACGAAGGGCAAGTCGCTG
TTTACCAGGATGTATACGCCGTCGACGGCCCCACCAGCCTGTACCACCAGGCCAACAAGGG
CGTGAGGGTGGCCTACTGGATCGGCTTCGACACCACACCCTTCATGTTCAAGAACCTGGCCG
GCGCCTACCCCAGCTACAGCACCAACTGGGCCGACGAGACAGTGCTGACCGCCAGGAACAT
CGGCCTGTGCAGCAGCGACGTGATGGAGAGGAGCCGGAGGGGCATGAGCATCCTGAGGAA
GAAGTACCTGAAGCCCAGCAACAACGTGCTGTTCAGCGTGGGCAGCACCATCTACCACGAG
AAGAGGGACCTGCTGAGGAGCTGGCACCTGCCCAGCGTGTTCCACCTGAGGGGCAAGCAGA
ACTACACCTGCAGGTGCGAGACAATCGTGAGCTGCGACGGCTACGTGGTGAAGAGGATCGC
CATCAGCCCCGGCCTGTACGGCAAGCCCAGCGGCTACGCCGCCACCATGCACAGGGAGGGC
TTCCTGTGCTGCAAGGTGACCGACACCCTGAACGGCGAGAGGGTGAGCTTCCCCGTGTGCA
CCTACGTGCCCGCCACCCTGTGCGACCAGATGACCGGCATCCTGGCCACCGACGTGAGCGC
CGACGACGCCCAGAAGCTGCTGGTGGGCCTGAACCAGAGGATCGTGGTGAACGGCAGGACC
CAGAGGAACACCAACACCATGAAGAACTACCTGCTGCCCGTGGTGGCCCAGGCCTTCGCCA
GGTGGGCCAAGGAGTACAAGGAGGACCAGGAGGACGAGAGGCCCCTGGGCCTGAGGGACC
GACAGCTGGTGATGGGCTGCTGCTGGGCCTTCAGGCGGCACAAGATCACCAGCATCTACAA
GAGGCCCGACACCCAGACCATCATCAAGGTGAACAGCGACTTCCACAGCTTCGTGCTGCCC
AGGATCGGCAGCAACACCCTGGAGATCGGCCTGAGGACCCGGATCAGGAAGATGCTGGAG
GAGCACAAGGAGCCCAGCCCTCTGATCACCGCCGAGGACGTGCAGGAGGCCAAGTGCGCC
GCCGACGAGGCCAAGGAGGTGAGGGAGGCCGAGGAGCTGAGGGCCGCCCTGCCTCCCCTG
GCCGCCGACGTGGAGGAGCCCACCCTGGAGGCCGACGTGGACCTGATGCTGCAGGAGGCCG
GCGCCGGCAGCGTGGAGACACCCAGGGGCCTGATCAAGGTGACCAGCTACGACGGCGAGG
ACAAGATCGGCAGCTACGCCGTGCTCAGCCCTCAGGCCGTGCTGAAGTCCGAGAAGCTGAG
CTGCATCCACCCTCTGGCCGAGCAGGTGATCGTGATCACCCACAGCGGCAGGAAGGGCAGG
TACGCCGTGGAGCCCTACCACGGCAAGGTGGTGGTCCCCGAGGGCCACGCCATCCCCGTGC
AGGACTTCCAGGCCCTGAGCGAGAGCGCCACCATCGTGTATAACGAGAGGGAGTTCGTGAA
CAGGTACCTGCACCACATCGCCACCCACGGCGGCGCCCTGAACACCGACGAGGAGTACTAC
AAGACCGTGAAGCCCAGCGAGCACGACGGCGAGTACCTGTACGACATCGACAGGAAGCAG
TGCGTGAAGAAGGAGCTGGTGACCGGCCTGGGCCTGACCGGCGAGCTGGTGGACCCTCCCT
TCCACGAGTTCGCCTACGAGAGCCTGAGGACCAGGCCCGCCGCTCCCTACCAGGTGCCCAC
CATCGGCGTGTACGGCGTGCCCGGCAGCGGCAAGAGCGGCATCATCAAGAGCGCCGTGACC
AAGAAGGACCTGGTGGTGAGCGCCAAGAAGGAGAACTGCGCCGAGATCATCAGGGACGTG
AAGAAGATGAAGGGCCTGGACGTGAACGCCAGGACCGTGGACAGCGTGCTCCTGAACGGCT
GCAAGCACCCCGTGGAGACACTGTATATCGACGAGGCCTTCGCCTGCCACGCCGGCACCCT
GAGGGCCCTGATCGCCATCATCAGGCCCAAGAAGGCCGTGCTGTGCGGCGACCCCAAGCAG
TGCGGCTTCTTCAACATGATGTGCCTGAAGGTGCACTTCAACCACGAGATCTGCACCCAGGT
GTTCCACAAGAGCATCAGCAGGCGGTGCACCAAGAGCGTGACCAGCGTGGTGAGCACCCTG
TTCTACGACAAGAAGATGAGGACCACCAACCCCAAGGAGACAAAGATCGTGATCGACACCA
CCGGCAGCACCAAGCCCAAGCAGGACGACCTGATCCTGACCTGCTTCAGGGGCTGGGTGAA
GCAGCTGCAGATCGACTACAAGGGCAACGAGATCATGACCGCCGCCGCTAGCCAGGGCCTG
ACCAGGAAGGGCGTGTACGCCGTGAGGTACAAGGTGAACGAGAATCCCCTGTACGCCCCTA
CCAGCGAGCACGTGAACGTCCTGCTGACCAGGACCGAGGACAGGATCGTGTGGAAGACCCT
GGCCGGCGACCCCTGGATCAAGACCCTGACCGCCAAGTACCCCGGCAACTTCACCGCCACC
ATCGAGGAGTGGCAGGCCGAGCACGACGCCATCATGAGGCACATCCTGGAGAGGCCCGACC
CCACCGACGTGTTCCAGAACAAGGCCAACGTGTGCTGGGCCAAGGCCCTGGTGCCCGTGCT
GAAGACCGCCGGCATCGACATGACCACCGAGCAGTGGAACACCGTGGACTACTTCGAGACA
GACAAGGCCCACAGCGCCGAGATCGTGCTGAACCAGCTGTGCGTGAGGTTCTTCGGCCTGG
ACCTGGACAGCGGCCTGTTCAGCGCCCCTACCGTGCCCCTGAGCATCAGGAACAACCACTG
GGACAACAGCCCCAGCCCCAACATGTACGGCCTGAACAAGGAGGTGGTGAGGCAGCTGAG
CAGGCGGTACCCTCAGCTGCCCAGGGCCGTGGCCACCGGCAGGGTGTACGACATGAACACC
GGCACCCTGAGGAACTACGACCCCAGGATCAACCTGGTGCCCGTGAACAGGCGGCTGCCAC
ACGCCCTGGTGCTGCACCACAACGAGCACCCTCAGAGCGACTTCAGCAGCTTCGTGAGCAA
GCTGAAGGGCAGGACCGTGCTGGTGGTGGGCGAGAAGCTGAGCGTGCCCGGCAAGATGGT
GGACTGGCTGAGCGACAGGCCCGAGGCCACCTTCCGGGCCAGGCTGGACCTGGGCATCCCC
GGCGACGTGCCCAAGTACGACATCATCTTCGTGAACGTGAGGACCCCTTACAAGTACCACC
ACTACCAGCAGTGCGAGGACCACGCCATCAAGCTGAGCATGCTGACCAAGAAGGCCTGCCT
GCACCTGAACCCCGGCGGCACCTGCGTGAGCATCGGCTACGGCTACGCCGACAGGGCCAGC
GAGAGCATCATCGGCGCCATCGCCAGGCTGTTCAAGTTCAGCAGGGTGTGCAAGCCCAAGA
GCAGCCTGGAGGAGACAGAGGTGCTGTTCGTGTTCATCGGCTACGACCGGAAGGCCAGGAC
CCACAACCCCTACAAGCTGAGCAGCACCCTGACCAACATCTACACCGGCAGCAGGCTGCAC
GAGGCCGGCTGCGCCCCTAGCTACCACGTGGTGAGGGGCGACATCGCCACCGCCACCGAGG
GCGTGATCATCAACGCCGCCAACAGCAAGGGCCAGCCCGGCGGCGGGGTGTGCGGCGCCCT
GTATAAGAAGTTCCCCGAGAGCTTCGACCTGCAGCCCATCGAGGTGGGCAAGGCCAGGCTG
GTGAAGGGCGCCGCCAAGCACATCATCCACGCCGTGGGCCCCAACTTCAACAAGGTGAGCG
AGGTGGAGGGCGACAAGCAGCTGGCCGAGGCCTACGAGAGCATCGCCAAGATCGTGAACG
ACAACAACTACAAGAGCGTGGCCATCCCTCTGCTGAGCACCGGCATCTTCAGCGGCAACAA
GGACAGGCTGACCCAGAGCCTGAACCACCTGCTGACCGCCCTGGACACCACCGACGCCGAC
GTGGCCATCTACTGCAGGGACAAGAAGTGGGAGATGACCCTGAAGGAGGCCGTGGCCAGG
CGGGAGGCCGTGGAGGAGATCTGCATCAGCGACGACAGCAGCGTGACGGAGCCCGACGCC
GAGCTGGTGAGGGTGCACCCCAAGAGCAGCCTGGCCGGCAGGAAGGGCTACAGCACCAGC
GACGGCAAGACCTTCAGCTACCTGGAGGGCACCAAGTTCCACCAGGCCGCCAAGGACATCG
CCGAGATCAACGCCATGTGGCCCGTGGCCACCGAGGCCAACGAGCAGGTGTGCATGTATAT
CCTGGGCGAGAGCATGAGCAGCATCAGGAGCAAGTGCCCCGTGGAGGAGAGCGAGGCCAG
CACCCCTCCCAGCACCCTGCCCTGCCTGTGCATCCACGCCATGACCCCTGAGAGGGTGCAGC
GGCTGAAGGCCAGCAGGCCCGAGCAGATCACCGTGTGCAGCAGCTTCCCTCTGCCCAAGTA
CCGGATCACCGGCGTGCAGAAGATCCAGTGCAGCCAGCCCATCCTGTTCAGCCCCAAGGTG
CCCGCCTACATCCACCCCAGGAAGTACCTGGTGGAGACACCCCCCGTGGACGAGACACCCG
AGCCCAGCGCCGAGAACCAGAGCACCGAGGGCACCCCTGAGCAGCCTCCCCTGATCACCGA
GGACGAGACAAGGACCAGGACGCCCGAGCCCATCATCATTGAGGAGGAAGAGGAGGACAG
CATCAGCCTGCTGAGCGACGGCCCCACCCACCAGGTGCTGCAGGTGGAGGCCGACATCCAC
GGCCCTCCCAGCGTGAGCAGCTCCAGCTGGAGCATCCCTCACGCCAGCGACTTCGACGTGG
ACAGCCTGAGCATCCTGGACACCCTGGAGGGCGCCAGCGTGACCAGCGGCGCCACCAGCGC
CGAGACAAACAGCTACTTCGCCAAGAGCATGGAGTTCCTGGCCAGGCCCGTGCCCGCCCCT
AGGACCGTGTTCAGGAACCCTCCCCACCCCGCCCCTAGGACCAGGACCCCTAGCCTGGCCC
CTAGCAGGGCCTGCAGCAGGACCAGCCTGGTGAGCACCCCTCCCGGCGTGAACCGGGTGAT
CACCAGGGAGGAGCTGGAGGCCCTGACCCCTAGCAGGACCCCTAGCAGGAGCGTGAGCAG
GACCAGCCTGGTGAGCAACCCTCCCGGCGTGAACCGGGTGATCACCAGGGAGGAGTTCGAG
GCCTTCGTGGCCCAGCAGCAAAGGCGGTTCGACGCCGGCGCCTACATCTTCAGCAGCGACA
CCGGCCAGGGCCACCTGCAGCAGAAGTCCGTGAGGCAGACCGTGCTGAGCGAGGTGGTCCT
GGAGAGGACGGAGCTGGAGATCAGCTACGCCCCTAGGCTGGACCAGGAGAAGGAGGAGCT
GCTGAGGAAGAAGCTGCAGCTGAACCCCACCCCTGCCAACAGGAGCAGGTACCAGAGCAG
GAAGGTGGAGAACATGAAGGCCATCACCGCCAGGCGGATCCTGCAGGGCCTGGGCCACTAC
CTGAAGGCCGAGGGCAAGGTGGAGTGCTACAGGACCCTGCACCCCGTGCCCCTGTACTCCA
GCTCCGTGAACAGGGCCTTCAGCAGCCCCAAGGTGGCCGTGGAGGCCTGCAACGCCATGCT
GAAGGAGAACTTCCCCACCGTGGCCAGCTACTGCATCATCCCCGAGTACGACGCCTACCTG
GACATGGTGGACGGCGCCAGCTGCTGCCTGGACACCGCCAGCTTCTGCCCCGCCAAGCTGA
GGAGCTTCCCCAAGAAGCACAGCTACCTGGAGCCCACCATCAGGAGCGCCGTGCCCAGCGC
CATCCAGAACACCCTGCAGAACGTGCTGGCCGCCGCTACCAAGAGGAACTGCAACGTGACC
CAGATGAGGGAGCTGCCCGTGCTGGACAGCGCCGCCTTCAACGTGGAGTGCTTCAAGAAGT
ACGCCTGCAACAACGAGTACTGGGAGACATTCAAGGAGAACCCCATCAGGCTGACCGAGGA
GAACGTGGTGAACTACATCACCAAGCTGAAGGGCCCCAAGGCCGCCGCTCTGTTCGCCAAG
ACCCACAACCTGAACATGCTCCAGGACATCCCTATGGACAGGTTCGTGATGGACCTGAAGA
GGGACGTGAAGGTGACCCCTGGCACCAAGCACACCGAGGAGAGGCCCAAGGTGCAGGTGA
TCCAGGCCGCCGACCCTCTGGCCACCGCCTACCTGTGCGGCATCCACAGGGAGCTGGTGAG
GCGGCTGAACGCCGTCCTGCTGCCCAACATCCACACCCTGTTCGACATGAGCGCCGAGGAC
TTCGACGCCATCATCGCCGAGCACTTCCAGCCCGGCGACTGCGTGCTGGAGACAGACATCG
CCAGCTTCGACAAGAGCGAGGACGACGCTATGGCCCTGACCGCCCTGATGATCCTGGAGGA
CCTGGGCGTGGACGCCGAGCTGCTGACCCTGATCGAGGCCGCCTTCGGCGAGATCAGCAGC
ATCCACCTGCCCACCAAGACCAAGTTCAAGTTCGGCGCCATGATGAAGTCCGGCATGTTCCT
GACCCTGTTCGTGAACACCGTGATCAACATCGTGATCGCCAGCAGGGTGCTGCGGGAGAGG
CTGACCGGCAGCCCCTGCGCCGCCTTCATCGGCGACGACAACATCGTGAAGGGCGTGAAGT
CCGACAAGCTGATGGCCGACAGGTGCGCCACCTGGCTGAACATGGAGGTGAAGATCATCGA
CGCCGTGGTGGGCGAGAAGGCCCCTTACTTCTGCGGCGGCTTCATCCTGTGCGACAGCGTGA
CCGGCACCGCCTGCAGGGTGGCCGACCCTCTGAAGAGGCTGTTCAAGCTGGGCAAGCCCCT
GGCCGCCGACGACGAGCACGACGACGATAGGCGGAGGGCCCTGCACGAGGAGAGCACCAG
GTGGAACCGGGTGGGCATCCTGAGCGAGCTGTGCAAGGCCGTGGAGAGCAGGTACGAGAC
AGTGGGCACCAGCATCATCGTGATGGCCATGACCACCCTGGCCAGCAGCGTCAAGTCCTTC
AGCTACCTGAGGGGGGCCCCTATAACTCTCTACGGCTAACCTGAATGGACTACGACATAGTC
TAGTCCGCCAAGGCCCTTAAGTTAAAACAGCCTGTGGGTTGATCCCACCCACAGGCCCATTG
GGCGCTAGCACTCTGGTATCACGGTACCTTTGTGCGCCTGTTTTATACCCCCTCCCCCAACTG
TAACTTAGAAGTAACACACACCGATCAACAGTCAGCGTGGCACACCAGCCACGTTTTGATC
AAGCACTTCTGTTACCCCGGACTGAGTATCAATAGACTGCTCACGCGGTTGAAGGAGAAAG
CGTTCGTTATCCGGCCAACTACTTCGAAAAACCTAGTAACACCGTGGAAGTTGCAGAGTGTT
TCGCTCAGCACTACCCCAGTGTAGATCAGGTCGATGAGTCACCGCATTCCCCACGGGCGACC
GTGGCGGTGGCTGCGTTGGCGGCCTGCCCATGGGGAAACCCATGGGACGCTCTAATACAGA
CATGGTGCGAAGAGTCTATTGAGCTAGTTGGTAGTCCTCCGGCCCCTGAATGCGGCTAATCC
TAACTGCGGAGCACACACCCTCAAGCCAGAGGGCAGTGTGTCGTAACGGGCAACTCTGCAG
CGGAACCGACTACTTTGGGTGTCCGTGTTTCATTTTATTCCTATACTGGCTGCTTATGGTGAC
AATTGAGAGATCGTTACCATATAGCTATTGGATTGGCCATCCGGTGACTAATAGAGCTATTA
TATATCCCTTTGTTGGGTTTATACCACTTAGCTTGAAAGAGGTTAAAACATTACAATTCATTG
TTAAGTTGAATACAGCAAAATGAATCCTAACCAGAAGATCATCACCATCGGCAGCGTGAGC
CTGACCATCAGCACCATCTGCTTCTTCATGCAGATCGCCATCCTGATCACCACCGTGACCCT
GCACTTCAAGCAGTACGAGTTCAACAGCCCTCCCAACAACCAGGTGATGCTGTGCGAGCCC
ACCATCATCGAGAGGAACATGACCGAGATCGTGTACCTGACCAACACCACCATCGAGAAGG
AGATCTGCCCCAAGCCCGCCGAGTACAGGAACTGGAGCAAGCCCCAGTGCGGCATCACCGG
CTTCGCCCCTTTCAGCAAGGACAACAGCATCAGGCTGAGCGCCGGCGGCGACATCTGGGTG
ACCAGGGAGCCCTACGTGAGCTGCGACCTGGACAAGTGCTACCAGTTCGCCCTGGGCCAGG
GCACCACCCTGAACAACGTGCACAGCAACAACACCGTGAGGGACAGGACCCCTTACCGGAC
CCTGCTGATGAACGAGCTGGGCGTGCCCTTCCACCTGGGCACCAAGCAGGTGTGCATCGCCT
GGAGCAGCTCCAGCTGCCACGACGGCAAGGCCTGGCTGCACGTGTGCATCACCGGCGACGA
CAAGAACGCCACCGCCAGCTTCATCTACAACGGCAGGCTGGTGGACAGCGTGGTGAGCTGG
AGCAACGACATCCTGAGGACCCAGGAGAGCGAGTGCGTGTGCATCAACGGCACCTGCACCG
TGGTGATGACCGACGGCAACGCCACCGGCAAGGCCGACACCAAGATCCTGTTCATCGAGGA
GGGCAAGATCGTGCACACCAGCAAGCTGAGCGGCAGCGCCCAGCACGTGGAGGAGTGCAG
CTGCTACCCCAGGTACCCCGGCGTGAGGTGCGTGTGCAGGGACAACTGGAAGGGCAGCAAC
CGGCCCATCATCGACATCAACATCAAGGACCACAGCATCGTCAGCAGGTACGTGTGCAGCG
GCCTGGTGGGCGACACCCCTAGAAAGAGCGACAGCAGCTCCAGCAGCCACTGCCTGAACCC
CAACAACGAGAAGGGCGACCACGGCGTGAAGGGCTGGGCCTTCGACGACGGCAACGACGT
GTGGATGGGCAGGACCATCAACGAGACAAGCAGGCTGGGCTACGAGACATTCAAGGTGGT
GGAGGGCTGGAGCAACCCCAAGAGCAAGCTGCAGATCAACAGGCAGGTGATCGTGGACAG
GGGCGACAGGAGCGGCTACAGCGGCATCTTCAGCGTGGAGGGCAAGAGCTGCATCAACCG
GTGCTTCTACGTGGAGCTGATCAGGGGCAGGAAGGAGGAGACAGAGGTGCTGTGGACCAGC
AACAGCATCGTGGTGTTCTGCGGCACCAGCGGCACCTACGGCACCGGCAGCTGGCCCGACG
GCGCCAACCTGAGCCTGATGCACATCTGATAGTAAACTCGAAGCAGTGTTAAATCATTCAGC
TACCTGAGAGGGGCCCCTATAACTCTCTACGGCTAACCTGAATGGACTACGACATAGTCTAG
TCCGCCAAGGCCGAATTCTTAAAACAGCTGTGGGTTGTCACCCACCCACAGGGTCCACTGGG
CGCTAGTACACTGGTATCTCGGTACCTTTGTACGCCTGTTTTATACCCCCTCCCTGATTTGCA
ACTTAGAAGCAACGCAAACCAGATCAATAGTAGGTGTGACATACCAGTCGCATCTTGATCA
AGCACTTCTGTATCCCCGGACCGAGTATCAATAGACTGTGCACACGGTTGAAGGAGAAAAC
GTCCGTTACCCGGCTAACTACTTCGAGAAGCCTAGTAACGCCATTGAAGTTGCAGAGTGTTT
CGCTCAGCACTCCCCCCGTGTAGATCAGGTCGATGAGTCACCGCATTCCCCACGGGCGACCG
TGGCGGTGGCTGCGTTGGCGGCCTGCCTATGGGGTAACCCATAGGACGCTCTAATACGGAC
ATGGCGTGAAGAGTCTATTGAGCTAGTTAGTAGTCCTCCGGCCCCTGAATGCGGCTAATCCT
AACTGCGGAGCACATACCCTTAATCCAAAGGGCAGTGTGTCGTAACGGGCAACTCTGCAGC
GGAACCGACTACTTTGGGTGTCCGTGTTTCTTTTTATTCTTGTATTGGCTGCTTATGGTGACA
ATTAAAGAATTGTTACCATATAGCTATTGGATTGGCCATCCAGTGTCAAACAGAGCTATTGT
ATATCTCTTTGTTGGATTCACACCTCTCACTCTTGAAACGTTACACACCCTCAATTACATTAT
ACTGCTGAACACGAAGCGATGAAGACCATCATCGCCCTGAGCTACATCCTGTGCCTGGTGTT
CGCCCAGAAGATCCCCGGCAATGACAACAGCACCGCCACCCTGTGCCTGGGCCACCACGCC
GTGCCCAACGGCACCATCGTGAAGACCATCACCAACGACAGGATCGAGGTGACCAACGCCA
CCGAGCTGGTGCAGAACAGCAGCATCGGCGAGATCTGCGACAGCCCTCACCAGATCCTGGA
CGGCGGCAACTGCACCCTGATCGACGCCCTGCTGGGCGACCCTCAGTGCGACGGCTTCCAG
AACAAGGAGTGGGACCTGTTCGTGGAGAGGAGCAGGGCCAACAGCAACTGCTACCCCTACG
ACGTGCCCGACTACGCCAGCCTGAGGAGCCTGGTGGCCAGCAGCGGCACCCTGGAGTTCAA
GAACGAGAGCTTCAACTGGACCGGCGTGAAGCAGAACGGCACCAGCAGCGCCTGCATCAG
GGGCAGCAGCTCCAGCTTCTTCAGCCGGCTGAATTGGCTCACCCACCTGAACTACACCTACC
CCGCCCTGAACGTGACCATGCCCAACAACGAGCAGTTCGACAAGCTGTATATCTGGGGCGT
GCACCACCCCAGCACCGACAAGGACCAGATCAGCCTGTTCGCCCAGCCCAGCGGCAGGATC
ACCGTGAGCACCAAGAGGAGCCAGCAGGCCGTGATCCCCAACATCGGCAGCAGGCCCAGG
ATCAGGGACATCCCCAGCAGGATCAGCATCTACTGGACCATCGTGAAGCCCGGCGACATCC
TGCTGATCAACAGCACCGGCAACCTGATCGCCCCTAGGGGCTACTTCAAGATCAGGAGCGG
CAAGAGCAGCATCATGAGGAGCGACGCCCCTATCGGCAAGTGCAAGAGCGAGTGCATCACC
CCTAACGGCAGCATCCCCAACGACAAGCCCTTCCAGAACGTGAACAGGATCACCTACGGCG
CCTGCCCCAGGTACGTGAAGCAGAGCACCCTGAAGCTGGCCACCGGCATGAGGAACGTGCC
CGAGAAGCAGACCAGGGGCATCTTCGGCGCCATCGCCGGCTTCATCGAGAACGGCTGGGAG
GGCATGGTGGACGGCTGGTACGGCTTCAGGCACCAGAACAGCGAGGGCAGGGGCCAGGCC
GCCGACCTGAAGTCCACCCAGGCCGCCATCGACCAGATCAACGGCAAGCTGAACAGGCTGA
TCGGCAAGACCAACGAGAAGTTCCACCAGATCGAGAAGGAGTTCAGCGAGGTGGAGGGCA
GGGTGCAGGACCTGGAGAAGTACGTGGAGGACACCAAGATCGACCTGTGGAGCTACAACG
CCGAGCTGCTGGTGGCCCTGGAGAACCAGCACACCATCGACCTGACCGACAGCGAGATGAA
CAAGCTGTTCGAGAAGACCAAGAAGCAGCTGAGGGAGAACGCCGAGGACATGGGCAACGG
CTGCTTCAAGATCTACCACAAGTGCGACAACGCCTGCATCGGCAGCATCAGGAACGAGACA
TATGACCACAACGTGTACCGGGACGAGGCCCTGAACAACCGGTTCCAGATCAAGGGCGTGG
AGCTGAAGTCCGGCTACAAGGACTGGATCCTGTGGATCAGCTTCGCCATGAGCTGCTTCCTG
CTGTGCATCGCCCTGCTGGGCTTCATCATGTGGGCCTGCCAGAAGGGCAACATCAGGTGCAA
CATCTGCATCTGATAGTAAACTCGAGTATGTTACGTGCAAAGGTGATTGTCACCCCCCGAAA
GACCATATTGTGACACACCCTCAGTATCACGCCCAAACATTTACAGCCGCGGTGTCAAAAAC
CGCGTGGACGTGGTTAACATCCCTGCTGGGAGGATCAGCCGTAATTATTATAATTGGCTTGG
TGCTGGCTACTATTGTGGCCATGTACGTGCTGACCAACCAGAAACATAATTGAATACAGCAG
CAATTGGCAAGCTGCTTACATAGAACTCGCGGCGATTGGCATGCCGCCTTAAAATTTTTATT
TTATTTTTTCTTTTCTTTTCCGAATCGGATTTTGTTTTTAATATTTC
SEQ ID NO: 161 (sgP EMCV NA sgP CVB3 HA)
ATGGGCGGCGCATGAGAGAAGCCCAGACCAATTACCTACCCAAAATGGAGAAAGTTCACGT
TGACATCGAGGAAGACAGCCCATTCCTCAGAGCTTTGCAGCGGAGCTTCCCGCAGTTTGAG
GTAGAAGCCAAGCAGGTCACTGATAATGACCATGCTAATGCCAGAGCGTTTTCGCATCTGG
CTTCAAAACTGATCGAAACGGAGGTGGACCCATCCGACACGATCCTTGACATTGGAAGTGC
GCCCGCCCGCAGAATGTATTCTAAGCACAAGTATCATTGTATCTGTCCGATGAGATGTGCGG
AAGATCCGGACAGATTGTATAAGTATGCAACTAAGCTGAAGAAAAACTGTAAGGAAATAAC
TGATAAGGAATTGGACAAGAAAATGAAGGAGCTGGCCGCCGTCATGAGCGACCCTGACCTG
GAAACTGAGACTATGTGCCTCCACGACGACGAGTCGTGTCGCTACGAAGGGCAAGTCGCTG
TTTACCAGGATGTATACGCCGTCGACGGCCCCACCAGCCTGTACCACCAGGCCAACAAGGG
CGTGAGGGTGGCCTACTGGATCGGCTTCGACACCACACCCTTCATGTTCAAGAACCTGGCCG
GCGCCTACCCCAGCTACAGCACCAACTGGGCCGACGAGACAGTGCTGACCGCCAGGAACAT
CGGCCTGTGCAGCAGCGACGTGATGGAGAGGAGCCGGAGGGGCATGAGCATCCTGAGGAA
GAAGTACCTGAAGCCCAGCAACAACGTGCTGTTCAGCGTGGGCAGCACCATCTACCACGAG
AAGAGGGACCTGCTGAGGAGCTGGCACCTGCCCAGCGTGTTCCACCTGAGGGGCAAGCAGA
ACTACACCTGCAGGTGCGAGACAATCGTGAGCTGCGACGGCTACGTGGTGAAGAGGATCGC
CATCAGCCCCGGCCTGTACGGCAAGCCCAGCGGCTACGCCGCCACCATGCACAGGGAGGGC
TTCCTGTGCTGCAAGGTGACCGACACCCTGAACGGCGAGAGGGTGAGCTTCCCCGTGTGCA
CCTACGTGCCCGCCACCCTGTGCGACCAGATGACCGGCATCCTGGCCACCGACGTGAGCGC
CGACGACGCCCAGAAGCTGCTGGTGGGCCTGAACCAGAGGATCGTGGTGAACGGCAGGACC
CAGAGGAACACCAACACCATGAAGAACTACCTGCTGCCCGTGGTGGCCCAGGCCTTCGCCA
GGTGGGCCAAGGAGTACAAGGAGGACCAGGAGGACGAGAGGCCCCTGGGCCTGAGGGACC
GACAGCTGGTGATGGGCTGCTGCTGGGCCTTCAGGCGGCACAAGATCACCAGCATCTACAA
GAGGCCCGACACCCAGACCATCATCAAGGTGAACAGCGACTTCCACAGCTTCGTGCTGCCC
AGGATCGGCAGCAACACCCTGGAGATCGGCCTGAGGACCCGGATCAGGAAGATGCTGGAG
GAGCACAAGGAGCCCAGCCCTCTGATCACCGCCGAGGACGTGCAGGAGGCCAAGTGCGCC
GCCGACGAGGCCAAGGAGGTGAGGGAGGCCGAGGAGCTGAGGGCCGCCCTGCCTCCCCTG
GCCGCCGACGTGGAGGAGCCCACCCTGGAGGCCGACGTGGACCTGATGCTGCAGGAGGCCG
GCGCCGGCAGCGTGGAGACACCCAGGGGCCTGATCAAGGTGACCAGCTACGACGGCGAGG
ACAAGATCGGCAGCTACGCCGTGCTCAGCCCTCAGGCCGTGCTGAAGTCCGAGAAGCTGAG
CTGCATCCACCCTCTGGCCGAGCAGGTGATCGTGATCACCCACAGCGGCAGGAAGGGCAGG
TACGCCGTGGAGCCCTACCACGGCAAGGTGGTGGTCCCCGAGGGCCACGCCATCCCCGTGC
AGGACTTCCAGGCCCTGAGCGAGAGCGCCACCATCGTGTATAACGAGAGGGAGTTCGTGAA
CAGGTACCTGCACCACATCGCCACCCACGGCGGCGCCCTGAACACCGACGAGGAGTACTAC
AAGACCGTGAAGCCCAGCGAGCACGACGGCGAGTACCTGTACGACATCGACAGGAAGCAG
TGCGTGAAGAAGGAGCTGGTGACCGGCCTGGGCCTGACCGGCGAGCTGGTGGACCCTCCCT
TCCACGAGTTCGCCTACGAGAGCCTGAGGACCAGGCCCGCCGCTCCCTACCAGGTGCCCAC
CATCGGCGTGTACGGCGTGCCCGGCAGCGGCAAGAGCGGCATCATCAAGAGCGCCGTGACC
AAGAAGGACCTGGTGGTGAGCGCCAAGAAGGAGAACTGCGCCGAGATCATCAGGGACGTG
AAGAAGATGAAGGGCCTGGACGTGAACGCCAGGACCGTGGACAGCGTGCTCCTGAACGGCT
GCAAGCACCCCGTGGAGACACTGTATATCGACGAGGCCTTCGCCTGCCACGCCGGCACCCT
GAGGGCCCTGATCGCCATCATCAGGCCCAAGAAGGCCGTGCTGTGCGGCGACCCCAAGCAG
TGCGGCTTCTTCAACATGATGTGCCTGAAGGTGCACTTCAACCACGAGATCTGCACCCAGGT
GTTCCACAAGAGCATCAGCAGGCGGTGCACCAAGAGCGTGACCAGCGTGGTGAGCACCCTG
TTCTACGACAAGAAGATGAGGACCACCAACCCCAAGGAGACAAAGATCGTGATCGACACCA
CCGGCAGCACCAAGCCCAAGCAGGACGACCTGATCCTGACCTGCTTCAGGGGCTGGGTGAA
GCAGCTGCAGATCGACTACAAGGGCAACGAGATCATGACCGCCGCCGCTAGCCAGGGCCTG
ACCAGGAAGGGCGTGTACGCCGTGAGGTACAAGGTGAACGAGAATCCCCTGTACGCCCCTA
CCAGCGAGCACGTGAACGTCCTGCTGACCAGGACCGAGGACAGGATCGTGTGGAAGACCCT
GGCCGGCGACCCCTGGATCAAGACCCTGACCGCCAAGTACCCCGGCAACTTCACCGCCACC
ATCGAGGAGTGGCAGGCCGAGCACGACGCCATCATGAGGCACATCCTGGAGAGGCCCGACC
CCACCGACGTGTTCCAGAACAAGGCCAACGTGTGCTGGGCCAAGGCCCTGGTGCCCGTGCT
GAAGACCGCCGGCATCGACATGACCACCGAGCAGTGGAACACCGTGGACTACTTCGAGACA
GACAAGGCCCACAGCGCCGAGATCGTGCTGAACCAGCTGTGCGTGAGGTTCTTCGGCCTGG
ACCTGGACAGCGGCCTGTTCAGCGCCCCTACCGTGCCCCTGAGCATCAGGAACAACCACTG
GGACAACAGCCCCAGCCCCAACATGTACGGCCTGAACAAGGAGGTGGTGAGGCAGCTGAG
CAGGCGGTACCCTCAGCTGCCCAGGGCCGTGGCCACCGGCAGGGTGTACGACATGAACACC
GGCACCCTGAGGAACTACGACCCCAGGATCAACCTGGTGCCCGTGAACAGGCGGCTGCCAC
ACGCCCTGGTGCTGCACCACAACGAGCACCCTCAGAGCGACTTCAGCAGCTTCGTGAGCAA
GCTGAAGGGCAGGACCGTGCTGGTGGTGGGCGAGAAGCTGAGCGTGCCCGGCAAGATGGT
GGACTGGCTGAGCGACAGGCCCGAGGCCACCTTCCGGGCCAGGCTGGACCTGGGCATCCCC
GGCGACGTGCCCAAGTACGACATCATCTTCGTGAACGTGAGGACCCCTTACAAGTACCACC
ACTACCAGCAGTGCGAGGACCACGCCATCAAGCTGAGCATGCTGACCAAGAAGGCCTGCCT
GCACCTGAACCCCGGCGGCACCTGCGTGAGCATCGGCTACGGCTACGCCGACAGGGCCAGC
GAGAGCATCATCGGCGCCATCGCCAGGCTGTTCAAGTTCAGCAGGGTGTGCAAGCCCAAGA
GCAGCCTGGAGGAGACAGAGGTGCTGTTCGTGTTCATCGGCTACGACCGGAAGGCCAGGAC
CCACAACCCCTACAAGCTGAGCAGCACCCTGACCAACATCTACACCGGCAGCAGGCTGCAC
GAGGCCGGCTGCGCCCCTAGCTACCACGTGGTGAGGGGCGACATCGCCACCGCCACCGAGG
GCGTGATCATCAACGCCGCCAACAGCAAGGGCCAGCCCGGCGGCGGGGTGTGCGGCGCCCT
GTATAAGAAGTTCCCCGAGAGCTTCGACCTGCAGCCCATCGAGGTGGGCAAGGCCAGGCTG
GTGAAGGGCGCCGCCAAGCACATCATCCACGCCGTGGGCCCCAACTTCAACAAGGTGAGCG
AGGTGGAGGGCGACAAGCAGCTGGCCGAGGCCTACGAGAGCATCGCCAAGATCGTGAACG
ACAACAACTACAAGAGCGTGGCCATCCCTCTGCTGAGCACCGGCATCTTCAGCGGCAACAA
GGACAGGCTGACCCAGAGCCTGAACCACCTGCTGACCGCCCTGGACACCACCGACGCCGAC
GTGGCCATCTACTGCAGGGACAAGAAGTGGGAGATGACCCTGAAGGAGGCCGTGGCCAGG
CGGGAGGCCGTGGAGGAGATCTGCATCAGCGACGACAGCAGCGTGACGGAGCCCGACGCC
GAGCTGGTGAGGGTGCACCCCAAGAGCAGCCTGGCCGGCAGGAAGGGCTACAGCACCAGC
GACGGCAAGACCTTCAGCTACCTGGAGGGCACCAAGTTCCACCAGGCCGCCAAGGACATCG
CCGAGATCAACGCCATGTGGCCCGTGGCCACCGAGGCCAACGAGCAGGTGTGCATGTATAT
CCTGGGCGAGAGCATGAGCAGCATCAGGAGCAAGTGCCCCGTGGAGGAGAGCGAGGCCAG
CACCCCTCCCAGCACCCTGCCCTGCCTGTGCATCCACGCCATGACCCCTGAGAGGGTGCAGC
GGCTGAAGGCCAGCAGGCCCGAGCAGATCACCGTGTGCAGCAGCTTCCCTCTGCCCAAGTA
CCGGATCACCGGCGTGCAGAAGATCCAGTGCAGCCAGCCCATCCTGTTCAGCCCCAAGGTG
CCCGCCTACATCCACCCCAGGAAGTACCTGGTGGAGACACCCCCCGTGGACGAGACACCCG
AGCCCAGCGCCGAGAACCAGAGCACCGAGGGCACCCCTGAGCAGCCTCCCCTGATCACCGA
GGACGAGACAAGGACCAGGACGCCCGAGCCCATCATCATTGAGGAGGAAGAGGAGGACAG
CATCAGCCTGCTGAGCGACGGCCCCACCCACCAGGTGCTGCAGGTGGAGGCCGACATCCAC
GGCCCTCCCAGCGTGAGCAGCTCCAGCTGGAGCATCCCTCACGCCAGCGACTTCGACGTGG
ACAGCCTGAGCATCCTGGACACCCTGGAGGGCGCCAGCGTGACCAGCGGCGCCACCAGCGC
CGAGACAAACAGCTACTTCGCCAAGAGCATGGAGTTCCTGGCCAGGCCCGTGCCCGCCCCT
AGGACCGTGTTCAGGAACCCTCCCCACCCCGCCCCTAGGACCAGGACCCCTAGCCTGGCCC
CTAGCAGGGCCTGCAGCAGGACCAGCCTGGTGAGCACCCCTCCCGGCGTGAACCGGGTGAT
CACCAGGGAGGAGCTGGAGGCCCTGACCCCTAGCAGGACCCCTAGCAGGAGCGTGAGCAG
GACCAGCCTGGTGAGCAACCCTCCCGGCGTGAACCGGGTGATCACCAGGGAGGAGTTCGAG
GCCTTCGTGGCCCAGCAGCAAAGGCGGTTCGACGCCGGCGCCTACATCTTCAGCAGCGACA
CCGGCCAGGGCCACCTGCAGCAGAAGTCCGTGAGGCAGACCGTGCTGAGCGAGGTGGTCCT
GGAGAGGACGGAGCTGGAGATCAGCTACGCCCCTAGGCTGGACCAGGAGAAGGAGGAGCT
GCTGAGGAAGAAGCTGCAGCTGAACCCCACCCCTGCCAACAGGAGCAGGTACCAGAGCAG
GAAGGTGGAGAACATGAAGGCCATCACCGCCAGGCGGATCCTGCAGGGCCTGGGCCACTAC
CTGAAGGCCGAGGGCAAGGTGGAGTGCTACAGGACCCTGCACCCCGTGCCCCTGTACTCCA
GCTCCGTGAACAGGGCCTTCAGCAGCCCCAAGGTGGCCGTGGAGGCCTGCAACGCCATGCT
GAAGGAGAACTTCCCCACCGTGGCCAGCTACTGCATCATCCCCGAGTACGACGCCTACCTG
GACATGGTGGACGGCGCCAGCTGCTGCCTGGACACCGCCAGCTTCTGCCCCGCCAAGCTGA
GGAGCTTCCCCAAGAAGCACAGCTACCTGGAGCCCACCATCAGGAGCGCCGTGCCCAGCGC
CATCCAGAACACCCTGCAGAACGTGCTGGCCGCCGCTACCAAGAGGAACTGCAACGTGACC
CAGATGAGGGAGCTGCCCGTGCTGGACAGCGCCGCCTTCAACGTGGAGTGCTTCAAGAAGT
ACGCCTGCAACAACGAGTACTGGGAGACATTCAAGGAGAACCCCATCAGGCTGACCGAGGA
GAACGTGGTGAACTACATCACCAAGCTGAAGGGCCCCAAGGCCGCCGCTCTGTTCGCCAAG
ACCCACAACCTGAACATGCTCCAGGACATCCCTATGGACAGGTTCGTGATGGACCTGAAGA
GGGACGTGAAGGTGACCCCTGGCACCAAGCACACCGAGGAGAGGCCCAAGGTGCAGGTGA
TCCAGGCCGCCGACCCTCTGGCCACCGCCTACCTGTGCGGCATCCACAGGGAGCTGGTGAG
GCGGCTGAACGCCGTCCTGCTGCCCAACATCCACACCCTGTTCGACATGAGCGCCGAGGAC
TTCGACGCCATCATCGCCGAGCACTTCCAGCCCGGCGACTGCGTGCTGGAGACAGACATCG
CCAGCTTCGACAAGAGCGAGGACGACGCTATGGCCCTGACCGCCCTGATGATCCTGGAGGA
CCTGGGCGTGGACGCCGAGCTGCTGACCCTGATCGAGGCCGCCTTCGGCGAGATCAGCAGC
ATCCACCTGCCCACCAAGACCAAGTTCAAGTTCGGCGCCATGATGAAGTCCGGCATGTTCCT
GACCCTGTTCGTGAACACCGTGATCAACATCGTGATCGCCAGCAGGGTGCTGCGGGAGAGG
CTGACCGGCAGCCCCTGCGCCGCCTTCATCGGCGACGACAACATCGTGAAGGGCGTGAAGT
CCGACAAGCTGATGGCCGACAGGTGCGCCACCTGGCTGAACATGGAGGTGAAGATCATCGA
CGCCGTGGTGGGCGAGAAGGCCCCTTACTTCTGCGGCGGCTTCATCCTGTGCGACAGCGTGA
CCGGCACCGCCTGCAGGGTGGCCGACCCTCTGAAGAGGCTGTTCAAGCTGGGCAAGCCCCT
GGCCGCCGACGACGAGCACGACGACGATAGGCGGAGGGCCCTGCACGAGGAGAGCACCAG
GTGGAACCGGGTGGGCATCCTGAGCGAGCTGTGCAAGGCCGTGGAGAGCAGGTACGAGAC
AGTGGGCACCAGCATCATCGTGATGGCCATGACCACCCTGGCCAGCAGCGTCAAGTCCTTC
AGCTACCTGAGGGGGGCCCCTATAACTCTCTACGGCTAACCTGAATGGACTACGACATAGTC
TAGTCCGCCAAGGCCCTTAAGCTCCCTCCCCCCCCCCTAACGTTACTGGCCGAAGCCGCTTG
GAATAAGGCCGGTGTGCGTTTGTCTATATGTTATTTTCCACCATATTGCCGTCTTTTGGCAAT
GTGAGGGCCCGGAAACCTGGCCCTGTCTTCTTGACGAGCATTCCTAGGGGTCTTTCCCCTCT
CGCCAAAGGAATGCAAGGTCTGTTGAATGTCGTGAAGGAAGCAGTTCCTCTGGAAGCTTCTT
GAAGACAAACAACGTCTGTAGCGACCCTTTGCAGGCAGCGGAACCCCCCACCTGGCGACAG
GTGCCTCTGCGGCCAAAAGCCACGTGTATAAGATACACCTGCAAAGGCGGCACAACCCCAG
TGCCACGTTGTGAGTTGGATAGTTGTGGAAAGAGTCAAATGGCTCTCCTCAAGCGTATTCAA
CAAGGGGCTGAAGGATGCCCAGAAGGTACCCCATTGTATGGGATCTGATCTGGGGCCTCGG
TGCACATGCTTTACGTGTGTTTAGTCGAGGTTAAAAAACGTCTAGGCCCCCCGAACCACGGG
GACGTGGTTTTCCTTTGAAAAACACGATGATAATATGAATCCTAACCAGAAGATCATCACCA
TCGGCAGCGTGAGCCTGACCATCAGCACCATCTGCTTCTTCATGCAGATCGCCATCCTGATC
ACCACCGTGACCCTGCACTTCAAGCAGTACGAGTTCAACAGCCCTCCCAACAACCAGGTGA
TGCTGTGCGAGCCCACCATCATCGAGAGGAACATGACCGAGATCGTGTACCTGACCAACAC
CACCATCGAGAAGGAGATCTGCCCCAAGCCCGCCGAGTACAGGAACTGGAGCAAGCCCCA
GTGCGGCATCACCGGCTTCGCCCCTTTCAGCAAGGACAACAGCATCAGGCTGAGCGCCGGC
GGCGACATCTGGGTGACCAGGGAGCCCTACGTGAGCTGCGACCTGGACAAGTGCTACCAGT
TCGCCCTGGGCCAGGGCACCACCCTGAACAACGTGCACAGCAACAACACCGTGAGGGACAG
GACCCCTTACCGGACCCTGCTGATGAACGAGCTGGGCGTGCCCTTCCACCTGGGCACCAAG
CAGGTGTGCATCGCCTGGAGCAGCTCCAGCTGCCACGACGGCAAGGCCTGGCTGCACGTGT
GCATCACCGGCGACGACAAGAACGCCACCGCCAGCTTCATCTACAACGGCAGGCTGGTGGA
CAGCGTGGTGAGCTGGAGCAACGACATCCTGAGGACCCAGGAGAGCGAGTGCGTGTGCATC
AACGGCACCTGCACCGTGGTGATGACCGACGGCAACGCCACCGGCAAGGCCGACACCAAG
ATCCTGTTCATCGAGGAGGGCAAGATCGTGCACACCAGCAAGCTGAGCGGCAGCGCCCAGC
ACGTGGAGGAGTGCAGCTGCTACCCCAGGTACCCCGGCGTGAGGTGCGTGTGCAGGGACAA
CTGGAAGGGCAGCAACCGGCCCATCATCGACATCAACATCAAGGACCACAGCATCGTCAGC
AGGTACGTGTGCAGCGGCCTGGTGGGCGACACCCCTAGAAAGAGCGACAGCAGCTCCAGCA
GCCACTGCCTGAACCCCAACAACGAGAAGGGCGACCACGGCGTGAAGGGCTGGGCCTTCGA
CGACGGCAACGACGTGTGGATGGGCAGGACCATCAACGAGACAAGCAGGCTGGGCTACGA
GACATTCAAGGTGGTGGAGGGCTGGAGCAACCCCAAGAGCAAGCTGCAGATCAACAGGCA
GGTGATCGTGGACAGGGGCGACAGGAGCGGCTACAGCGGCATCTTCAGCGTGGAGGGCAA
GAGCTGCATCAACCGGTGCTTCTACGTGGAGCTGATCAGGGGCAGGAAGGAGGAGACAGA
GGTGCTGTGGACCAGCAACAGCATCGTGGTGTTCTGCGGCACCAGCGGCACCTACGGCACC
GGCAGCTGGCCCGACGGCGCCAACCTGAGCCTGATGCACATCTGATAGTAAACTCGAAGCA
GTGTTAAATCATTCAGCTACCTGAGAGGGGCCCCTATAACTCTCTACGGCTAACCTGAATGG
ACTACGACATAGTCTAGTCCGCCAAGGCCGAATTCTTAAAACAGCCTGTGGGTTGATCCCAC
CCACAGGCCCATTGGGCGCTAGCACTCTGGTATCACGGTACCTTTGTGCGCCTGTTTTATAC
CCCCTCCCCCAACTGTAACTTAGAAGTAACACACACCGATCAACAGTCAGCGTGGCACACC
AGCCACGTTTTGATCAAGCACTTCTGTTACCCCGGACTGAGTATCAATAGACTGCTCACGCG
GTTGAAGGAGAAAGCGTTCGTTATCCGGCCAACTACTTCGAAAAACCTAGTAACACCGTGG
AAGTTGCAGAGTGTTTCGCTCAGCACTACCCCAGTGTAGATCAGGTCGATGAGTCACCGCAT
TCCCCACGGGCGACCGTGGCGGTGGCTGCGTTGGCGGCCTGCCCATGGGGAAACCCATGGG
ACGCTCTAATACAGACATGGTGCGAAGAGTCTATTGAGCTAGTTGGTAGTCCTCCGGCCCCT
GAATGCGGCTAATCCTAACTGCGGAGCACACACCCTCAAGCCAGAGGGCAGTGTGTCGTAA
CGGGCAACTCTGCAGCGGAACCGACTACTTTGGGTGTCCGTGTTTCATTTTATTCCTATACTG
GCTGCTTATGGTGACAATTGAGAGATCGTTACCATATAGCTATTGGATTGGCCATCCGGTGA
CTAATAGAGCTATTATATATCCCTTTGTTGGGTTTATACCACTTAGCTTGAAAGAGGTTAAA
ACATTACAATTCATTGTTAAGTTGAATACAGCAAAATGAAGACCATCATCGCCCTGAGCTAC
ATCCTGTGCCTGGTGTTCGCCCAGAAGATCCCCGGCAATGACAACAGCACCGCCACCCTGTG
CCTGGGCCACCACGCCGTGCCCAACGGCACCATCGTGAAGACCATCACCAACGACAGGATC
GAGGTGACCAACGCCACCGAGCTGGTGCAGAACAGCAGCATCGGCGAGATCTGCGACAGCC
CTCACCAGATCCTGGACGGCGGCAACTGCACCCTGATCGACGCCCTGCTGGGCGACCCTCA
GTGCGACGGCTTCCAGAACAAGGAGTGGGACCTGTTCGTGGAGAGGAGCAGGGCCAACAG
CAACTGCTACCCCTACGACGTGCCCGACTACGCCAGCCTGAGGAGCCTGGTGGCCAGCAGC
GGCACCCTGGAGTTCAAGAACGAGAGCTTCAACTGGACCGGCGTGAAGCAGAACGGCACCA
GCAGCGCCTGCATCAGGGGCAGCAGCTCCAGCTTCTTCAGCCGGCTGAATTGGCTCACCCAC
CTGAACTACACCTACCCCGCCCTGAACGTGACCATGCCCAACAACGAGCAGTTCGACAAGC
TGTATATCTGGGGCGTGCACCACCCCAGCACCGACAAGGACCAGATCAGCCTGTTCGCCCA
GCCCAGCGGCAGGATCACCGTGAGCACCAAGAGGAGCCAGCAGGCCGTGATCCCCAACATC
GGCAGCAGGCCCAGGATCAGGGACATCCCCAGCAGGATCAGCATCTACTGGACCATCGTGA
AGCCCGGCGACATCCTGCTGATCAACAGCACCGGCAACCTGATCGCCCCTAGGGGCTACTT
CAAGATCAGGAGCGGCAAGAGCAGCATCATGAGGAGCGACGCCCCTATCGGCAAGTGCAA
GAGCGAGTGCATCACCCCTAACGGCAGCATCCCCAACGACAAGCCCTTCCAGAACGTGAAC
AGGATCACCTACGGCGCCTGCCCCAGGTACGTGAAGCAGAGCACCCTGAAGCTGGCCACCG
GCATGAGGAACGTGCCCGAGAAGCAGACCAGGGGCATCTTCGGCGCCATCGCCGGCTTCAT
CGAGAACGGCTGGGAGGGCATGGTGGACGGCTGGTACGGCTTCAGGCACCAGAACAGCGA
GGGCAGGGGCCAGGCCGCCGACCTGAAGTCCACCCAGGCCGCCATCGACCAGATCAACGGC
AAGCTGAACAGGCTGATCGGCAAGACCAACGAGAAGTTCCACCAGATCGAGAAGGAGTTC
AGCGAGGTGGAGGGCAGGGTGCAGGACCTGGAGAAGTACGTGGAGGACACCAAGATCGAC
CTGTGGAGCTACAACGCCGAGCTGCTGGTGGCCCTGGAGAACCAGCACACCATCGACCTGA
CCGACAGCGAGATGAACAAGCTGTTCGAGAAGACCAAGAAGCAGCTGAGGGAGAACGCCG
AGGACATGGGCAACGGCTGCTTCAAGATCTACCACAAGTGCGACAACGCCTGCATCGGCAG
CATCAGGAACGAGACATATGACCACAACGTGTACCGGGACGAGGCCCTGAACAACCGGTTC
CAGATCAAGGGCGTGGAGCTGAAGTCCGGCTACAAGGACTGGATCCTGTGGATCAGCTTCG
CCATGAGCTGCTTCCTGCTGTGCATCGCCCTGCTGGGCTTCATCATGTGGGCCTGCCAGAAG
GGCAACATCAGGTGCAACATCTGCATCTGATAGTAAACTCGAGTATGTTACGTGCAAAGGT
GATTGTCACCCCCCGAAAGACCATATTGTGACACACCCTCAGTATCACGCCCAAACATTTAC
AGCCGCGGTGTCAAAAACCGCGTGGACGTGGTTAACATCCCTGCTGGGAGGATCAGCCGTA
ATTATTATAATTGGCTTGGTGCTGGCTACTATTGTGGCCATGTACGTGCTGACCAACCAGAA
ACATAATTGAATACAGCAGCAATTGGCAAGCTGCTTACATAGAACTCGCGGCGATTGGCAT
GCCGCCTTAAAATTTTTATTTTATTTTTTCTTTTCTTTTCCGAATCGGATTTTGTTTTTAATATT
TC
SEQ ID NO: 162 (CVB3 IRES version 1)
ACTCGACCGGAAACGCAATAGCCGAAAAACAAAAAACAAAAAAAACAAAAAAAAAACCA
AAAAAACAAAACACATTAAAACAGCCTGTGGGTTGATCCCACCCACAGGCCCATTGGGCGC
TAGCACTCTGGTATCACGGTACCTTTGTGCGCCTGTTTTATACCCCCTCCCCCAACTGTAACT
TAGAAGTAACACACACCGATCAACAGTCAGCGTGGCACACCAGCCACGTTTTGATCAAGCA
CTTCTGTTACCCCGGACTGAGTATCAATAGACTGCTCACGCGGTTGAAGGAGAAAGCGTTCG
TTATCCGGCCAACTACTTCGAAAAACCTAGTAACACCGTGGAAGTTGCAGAGTGTTTCGCTC
AGCACTACCCCAGTGTAGATCAGGTCGATGAGTCACCGCATTCCCCACGGGCGACCGTGGC
GGTGGCTGCGTTGGCGGCCTGCCCATGGGGAAACCCATGGGACGCTCTAATACAGACATGG
TGCGAAGAGTCTATTGAGCTAGTTGGTAGTCCTCCGGCCCCTGAATGCGGCTAATCCTAACT
GCGGAGCACACACCCTCAAGCCAGAGGGCAGTGTGTCGTAACGGGCAACTCTGCAGCGGAA
CCGACTACTTTGGGTGTCCGTGTTTCATTTTATTCCTATACTGGCTGCTTATGGTGACAATTG
AGAGATCGTTACCATATAGCTATTGGATTGGCCATCCGGTGACTAATAGAGCTATTATATAT
CCCTTTGTTGGGTTTATACCACTTAGCTTGAAAGAGGTTAAAACATTACAATTCATTGTTAA
GTTGAATACAGCAAA
SEQ ID NO: 163 (CVB3 IRES version 2)
TTAAAACAGCCTGTGGGTTGATCCCACCCACAGGCCCATTGGGCGCTAGCACTCTGGTATCA
CGGTACCTTTGTGCGCCTGTTTTATACCCCCTCCCCCAACTGTAACTTAGAAGTAACACACA
CCGATCAACAGTCAGCGTGGCACACCAGCCACGTTTTGATCAAGCACTTCTGTTACCCCGGA
CTGAGTATCAATAGACTGCTCACGCGGTTGAAGGAGAAAGCGTTCGTTATCCGGCCAACTA
CTTCGAAAAACCTAGTAACACCGTGGAAGTTGCAGAGTGTTTCGCTCAGCACTACCCCAGTG
TAGATCAGGTCGATGAGTCACCGCATTCCCCACGGGCGACCGTGGCGGTGGCTGCGTTGGC
GGCCTGCCCATGGGGAAACCCATGGGACGCTCTAATACAGACATGGTGCGAAGAGTCTATT
GAGCTAGTTGGTAGTCCTCCGGCCCCTGAATGCGGCTAATCCTAACTGCGGAGCACACACCC
TCAAGCCAGAGGGCAGTGTGTCGTAACGGGCAACTCTGCAGCGGAACCGACTACTTTGGGT
GTCCGTGTTTCATTTTATTCCTATACTGGCTGCTTATGGTGACAATTGAGAGATCGTTACCAT
ATAGCTATTGGATTGGCCATCCGGTGACTAATAGAGCTATTATATATCCCTTTGTTGGGTTTA
TACCACTTAGCTTGAAAGAGGTTAAAACATTACAATTCATTGTTAAGTTGAATACAGCAAA
SEQ ID NO: 164 (EMC VIRES)
TCCCTCCCCCCCCCCTAACGTTACTGGCCGAAGCCGCTTGGAATAAGGCCGGTGTGCGTTTG
TCTATATGTTATTTTCCACCATATTGCCGTCTTTTGGCAATGTGAGGGCCCGGAAACCTGGCC
CTGTCTTCTTGACGAGCATTCCTAGGGGTCTTTCCCCTCTCGCCAAAGGAATGCAAGGTCTG
TTGAATGTCGTGAAGGAAGCAGTTCCTCTGGAAGCTTCTTGAAGACAAACAACGTCTGTAGC
GACCCTTTGCAGGCAGCGGAACCCCCCACCTGGCGACAGGTGCCTCTGCGGCCAAAAGCCA
CGTGTATAAGATACACCTGCAAAGGCGGCACAACCCCAGTGCCACGTTGTGAGTTGGATAG
TTGTGGAAAGAGTCAAATGGCTCTCCTCAAGCGTATTCAACAAGGGGCTGAAGGATGCCCA
GAAGGTACCCCATTGTATGGGATCTGATCTGGGGCCTCGGTGCACATGCTTTACGTGTGTTT
AGTCGAGGTTAAAAAACGTCTAGGCCCCCCGAACCACGGGGACGTGGTTTTCCTTTGAAAA
ACACGATGATAA
SEQ ID NO: 165 (EV71 IRES)
TTAAAACAGCTGTGGGTTGTCACCCACCCACAGGGTCCACTGGGCGCTAGTACACTGGTATC
TCGGTACCTTTGTACGCCTGTTTTATACCCCCTCCCTGATTTGCAACTTAGAAGCAACGCAAA
CCAGATCAATAGTAGGTGTGACATACCAGTCGCATCTTGATCAAGCACTTCTGTATCCCCGG
ACCGAGTATCAATAGACTGTGCACACGGTTGAAGGAGAAAACGTCCGTTACCCGGCTAACT
ACTTCGAGAAGCCTAGTAACGCCATTGAAGTTGCAGAGTGTTTCGCTCAGCACTCCCCCCGT
GTAGATCAGGTCGATGAGTCACCGCATTCCCCACGGGCGACCGTGGCGGTGGCTGCGTTGG
CGGCCTGCCTATGGGGTAACCCATAGGACGCTCTAATACGGACATGGCGTGAAGAGTCTAT
TGAGCTAGTTAGTAGTCCTCCGGCCCCTGAATGCGGCTAATCCTAACTGCGGAGCACATACC
CTTAATCCAAAGGGCAGTGTGTCGTAACGGGCAACTCTGCAGCGGAACCGACTACTTTGGG
TGTCCGTGTTTCTTTTTATTCTTGTATTGGCTGCTTATGGTGACAATTAAAGAATTGTTACCA
TATAGCTATTGGATTGGCCATCCAGTGTCAAACAGAGCTATTGTATATCTCTTTGTTGGATTC
ACACCTCTCACTCTTGAAACGTTACACACCCTCAATTACATTATACTGCTGAACACGAAGCG

Unless defined otherwise, all technical and scientific terms used herein have the same meaning as is commonly understood by one of skill in the art to which this invention belongs.

Any and all references and citations to other documents, such as patents, patent applications, patent publications, journals, books, papers, web contents, that have been made throughout this disclosure are hereby incorporated herein in their entirety for all purposes. Although the invention has been described with reference to the above examples, it will be understood that modifications and variations are encompassed within the spirit and scope of the invention. Accordingly, the invention is limited only by the following claims.

1. A composition comprising one or more RNA molecules, wherein the one or more RNA molecules collectively encode a hemagglutinin (HA) polypeptide and a neuraminidase (NA) polypeptide of each of four different strains of influenza virus.

2. The composition of claim 1, wherein for each of the four different strains of influenza virus, the HA polypeptide and NA polypeptide are encoded by the same RNA molecule.

3. The composition of claim 1, wherein the HA and NA polypeptides of a first strain of influenza virus are encoded by a first RNA molecule, the HA and NA polypeptides of a second strain of influenza virus are encoded by a second RNA molecule, the HA and NA polypeptides of a third strain of influenza virus are encoded by a third RNA molecule, and the HA and NA polypeptides of a fourth strain of influenza virus are encoded by a fourth RNA molecule.

4. The composition of claim 3, wherein the first, second, third, and fourth RNA molecules are present in an equimolar ratio.

5. The composition of claim 1, wherein
(a) each of the one or more RNA molecules further encodes one or more viral replication proteins; or
(b) each of the one or more RNA molecules further encodes one or more viral replication proteins, wherein the one or more viral replication proteins are alphavirus proteins; or
(c) each of the one or more RNA molecules further encodes one or more viral replication proteins, wherein the one or more viral replication proteins are from Venezuelan Equine Encephalitis Virus (VEEV); or
(d) each of the one or more RNA molecules further encodes one or more viral replication proteins; wherein the one or more viral replication proteins comprise an alphavirus nonstructural protein 1 (nsP1), an alphavirus nonstructural protein 2 (nsP2), an alphavirus nonstructural protein 3 (nsP3), an alphavirus nonstructural protein 4 (nsP4), or any combination thereof; or
(e) each of the one or more RNA molecules encodes in 5′ to 3′ order: (i) one or more viral replication proteins. (ii) one of the NA polypeptides; and (iii) one of the HA polypeptides.

6.-9. (canceled)

10. The composition of claim 5, wherein the one or more viral replication proteins comprises a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, or 100% identity to the RNA sequence encoded by SEQ ID NO: 13.

11. The composition of claim 5, wherein sequences encoding the HA and NA polypeptides of at least one of the one or more RNA molecules are preceded by a subgenomic promoter (sgP).

12. The composition of claim 1, wherein
(a) each HA polypeptide comprises an antigenic fragment of a respective HA protein; or
(b) each NA polypeptide comprises an antigenic fragment of a respective NA protein.

13. (canceled)

14. The composition of claim 1, wherein the four different strains of influenza virus comprise one or more of H1N1, H3N2, Victoria-B, or Yamagata-B.

15. The composition of claim 14, wherein the four different strains of influenza virus comprise Victoria B/Austria/1359417/2021, H3N2 A/Darwin/6/2021, H1N1 A/Wisconsin/588/2019, and Yamagata B/PHUKET/3073/2013.

16. The composition of claim 1, wherein
(a) each of the one or more RNA molecules further comprise a 5′ untranslated region (UTR); or
(b) each of the one or more RNA molecules further comprise a 5′ untranslated region (UTR); wherein at least one 5′ UTR comprises a viral 5′ UTR, a non-viral 5′ UTR, or a combination of viral and non-viral 5′ UTR sequences; or
(c) each of the one or more RNA molecules further comprise a 5′ untranslated region (UTR); wherein at least one 5′ UTR comprises a viral 5′ UTR, a non-viral 5′ UTR, or a combination of viral and non-viral 5′ UTR sequences; and wherein the at least one 5′ UTR comprises an alphavirus 5′ UTR; or
(d) each of the one or more RNA molecules further comprise a 5′ untranslated region (UTR); wherein at least one 5′ UTR comprises a viral 5′ UTR, a non-viral 5′ UTR, or a combination of viral and non-viral 5′ UTR sequences; and wherein the at least one 5′ UTR comprises a Venezuelan Equine Encephalitis Virus (VEEV) 5′ UTR sequence.

17.-19. (canceled)

20. The composition of claim 1, wherein each of the one or more RNA molecules further comprise a 5′ untranslated region (UTR), wherein at least one 5′ UTR comprises the RNA sequence encoded by SEQ ID NO: 14.

21. The composition of claim 1, wherein
(a) each of the one or more RNA molecules further comprise a 3′ untranslated region (UTR); or
(b) each of the one or more RNA molecules further comprise a 3′ untranslated region (UTR); wherein at least one 3′ UTR comprises a viral 3′ UTR, a non-viral 3′ UTR, or a combination of viral and non-viral 3′ UTR sequences; or
(c) each of the one or more RNA molecules further comprise a 3′ untranslated region (UTR); wherein at least one 3′ UTR comprises a viral 3′ UTR, a non-viral 3′ UTR, or a combination of viral and non-viral 3′ UTR sequences; wherein the at least one 3′ UTR comprises an alphavirus 3′ UTR sequence; or
(d) each of the one or more RNA molecules further comprise a 3′ untranslated region (UTR); wherein at least one 3′ UTR comprises a viral 3′ UTR, a non-viral 3′ UTR, or a combination of viral and non-viral 3′ UTR sequences; wherein the at least one 3′ UTR comprises a Venezuelan Equine Encephalitis Virus (VEEV) 3′ UTR sequence; or
(e) each of the one or more RNA molecules further comprises a poly-A tail; or
(f) both (a) and (e).

22.-24. (canceled)

25. The composition of claim 1, wherein each of the one or more RNA molecules further comprise a 3′ untranslated region (UTR); wherein at least one 3′ UTR comprises the RNA sequence encoded by SEQ ID NO: 15.

26. (canceled)

27. The composition of claim 1, wherein the one or more RNA molecules are self-replicating RNA molecules.

28. The composition of claim 27, wherein the one or more RNA molecules comprise a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, or 100% identity to the RNA sequence encoded by any of SEQ ID NOs:1-4.

29. A composition comprising one or more DNA molecules encoding the one or more RNA molecules of the composition of claim 1.

30. The composition of claim 29, wherein
(a) each of the one or more DNA molecules comprises a promoter; or
(b) each of the one or more DNA molecules comprises a promoter located 5′ of a 5′ UTR; or
(c) each of the one or more DNA molecules comprises a promoter, wherein the promoter is a T7 promoter.

31. (canceled)

32. (canceled)

33. A composition comprising
(I)
(i) an RNA molecule comprising a sequence with at least 80% sequence identity to the RNA sequence encoded by SEQ ID NO: 5; or
(ii) an RNA molecule comprising a sequence with at least 80% sequence identity to the RNA sequence encoded by SEO ID NO: 9; or
(iii) both (i) and (ii); or
(II)
(i) an RNA molecule comprising a sequence with at least 80% sequence identity to the RNA sequence encoded by SEO ID NO: 6; or
(ii) an RNA molecule comprising a sequence with at least 80% sequence identity to the RNA sequence encoded by SEO ID NO: 10; or
(iii) both (i) and (ii); or
(III)
(i) an RNA molecule comprising a sequence with at least 80% sequence identity to the RNA sequence encoded by SEO ID NO: 7; or
(ii) an RNA molecule comprising a sequence with at least 80% sequence identity to the RNA sequence encoded by SEO ID NO: 11; or
(iii) both (i) and (ii); or
(IV)
(i) an RNA molecule comprising a sequence with at least 80% sequence identity to the RNA sequence encoded by SEO ID NO: 8; or
(ii) an RNA molecule comprising a sequence with at least 80% sequence identity to the RNA sequence encoded by SEO ID NO:12; or
(iii) both (i) and (ii); or
(V) an RNA molecule comprising a sequence with at least 80% sequence identity to the RNA sequence encoded by any of SEO ID NOs: 1-4.

34.-45. (canceled)

46. The composition of claim 1, further comprising an ionizable cationic lipid.

47. The composition of claim 46, wherein
(a) the ionizable cationic lipid has a structure of Formula I: 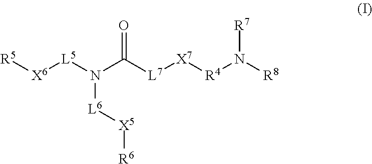
or a pharmaceutically acceptable salt or solvate thereof, wherein R⁵ and R⁶ are each independently selected from the group consisting of a linear or branched C₁-C₃₁ alkyl, C₂-C₃₁ alkenyl or C₂-C₃₁ alkynyl and cholesteryl; L⁵ and L⁶ are each independently selected from the group consisting of a linear C₁-C₂₀ alkyl and C₂-C₂₀ alkenyl; X⁵ is —C(O)O—, whereby —C(O)O—R⁶ is formed or —OC(O)— whereby —OC(O)—R⁶ is formed; X⁶ is —C(O)O— whereby —C(O)O—R⁵ is formed or —OC(O)— whereby —OC(O)—R⁵ is formed; X⁷ is S or O; L⁷ is absent or lower alkyl; R⁴ is a linear or branched C₁-C₆ alkyl; and R⁷ and R⁸ are each independently selected from the group consisting of a hydrogen and a linear or branched C₁-C₆ alkyl; or
(b) the ionizable cationic lipid is selected from Table 6; or
(c) the ionizable cationic lipid is ATX-126: 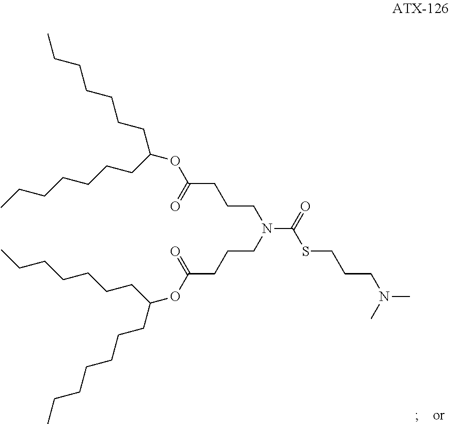 [SMILES: CCCCCCCC(CCCCCCC)OC(=O)CCCN(CCCC(=O)OC(CCCCCCC)CCCCCCC)C(=O)SCCCN(C)C]
(d) the ionizable cationic lipid is ATX-240:

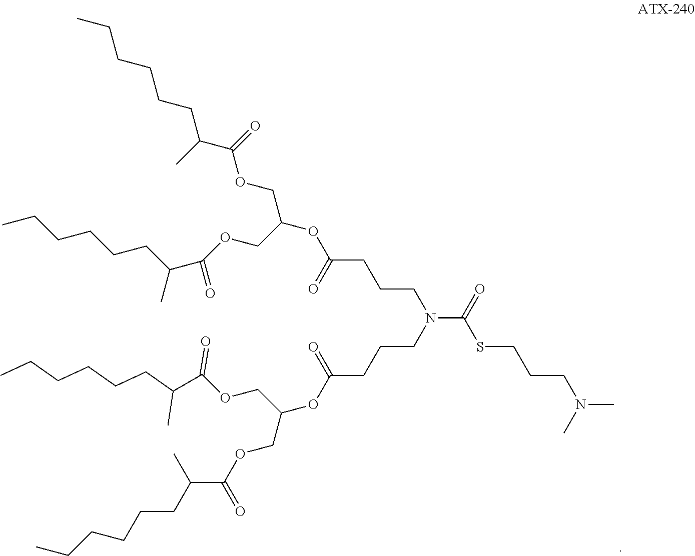 [SMILES: CCCCCCC(C)C(=O)OCC(COC(=O)C(C)CCCCCC)OC(=O)CCCN(CCCC(=O)OC(COC(=O)C(C)CCCCCC)COC(=O)C(C)CCCCCC)C(=O)SCCCN(C)C]

48.-50. (canceled)

51. The composition of claim 46, wherein the composition comprises a nitrogen to phosphate ratio (N:P) of about 5:1 to about 7:1.

52. A method of vaccinating a subject against influenza, the method comprising administering to the subject a composition of claim 1.

53. (canceled)

54. A composition comprising (i) a polynucleotide having a length of about 5,000 to about 20,000 nucleotides, and (ii) an ionizable cationic lipid, wherein the composition comprises a nitrogen to phosphate ratio (N:P) of (a) about 5:1 to about 7:1 or (b) about 7:1.

55. (canceled)
